(19) United States
(12) Patent Application Publication
(10) Pub. No.: US 2026/0308168 A1
(43) Pub. Date: Oct. 8, 2026

(54) ORGANIC ELECTROLUMINESCENT MATERIALS AND DEVICES

(71) Applicant: UNIVERSAL DISPLAY CORPORATION, Ewing, NJ (US)

(72) Inventors: Rasha HAMZE, Philadelphia, PA (US); Sean Michael Ryno, Yardley, PA (US); Elena Sheina, Yardley, PA (US); Hsiao-Fan Chen, Princeton, NJ (US); Dmitry Sergeyevich Andrianov, Landenberg, PA (US); Kelly R. Lewis, Downingtown, PA (US); Charles J. Stanton, Wilmington, DE (US)

(73) Assignee: UNIVERSAL DISPLAY CORPORATION, Ewing, NJ (US)

(21) Appl. No.: 19/562,990

(22) Filed: Mar. 11, 2026

Related U.S. Application Data
(60) Provisional application No. 63/773,564, filed on Mar. 18, 2025.

(51) Int. Cl. C07D 491/22; H10K 85/60; H10K 101/30

(52) U.S. Cl. CPC C07D 491/22; H10K 85/657; H10K 2101/30

(57) ABSTRACT

Provided are organic compounds comprising a 6-membered carbocyclic ring to which four 5-membered heterocyclic rings each comprising exactly one nitrogen atom are fused. Also provided are formulations comprising these organic compounds. Further provided are organic light emitting devices (OLEDs) as well as related consumer products that utilize these organic compounds.

### CROSS-REFERENCE TO RELATED APPLICATIONS

This application claims priority under 35 U.S.C. § 119(e) to U.S. Provisional Application No. 63/773,564, filed on Mar. 18, 2025, the entire contents of which are incorporated herein by reference.

### FIELD

The present disclosure generally relates to novel device architectures and the OLED devices having those novel architectures and their uses.

### BACKGROUND

Opto-electronic devices that make use of organic materials are becoming increasingly desirable for various reasons. Many of the materials used to make such devices are relatively inexpensive, so organic opto-electronic devices have the potential for cost advantages over inorganic devices. In addition, the inherent properties of organic materials, such as their flexibility, may make them well suited for particular applications such as fabrication on a flexible substrate. Examples of organic opto-electronic devices include organic light emitting diodes/devices (OLEDs), organic phototransistors, organic photovoltaic cells, organic scintillators, and organic photodetectors. For OLEDs, the organic materials may have performance advantages over conventional materials.

OLEDs make use of thin organic films that emit light when voltage is applied across the device. OLEDs are becoming an increasingly interesting technology for use in applications such as displays, illumination, and backlighting.

One application for emissive molecules is a full color display. Industry standards for such a display call for pixels adapted to emit particular colors, referred to as “saturated” colors. In particular, these standards call for saturated red, green, and blue pixels. Alternatively, the OLED can be designed to emit white light. In conventional liquid crystal displays emission from a white backlight is filtered using absorption filters to produce red, green and blue emission. The same technique can also be used with OLEDs. The white OLED can be either a single emissive layer (EML) device or a stack structure. Color may be measured using CIE coordinates, which are well known to the art.

### SUMMARY

In one aspect, the present disclosure provides a compound of Formula I:

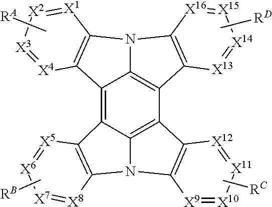

wherein each of X¹ to X¹⁶ is independently C or N;
each of R^{A}, R^{B}, R^{C}, and R^{D} independently represents mono to the maximum allowable substitutions, or no substitutions;
wherein any two of R^{A}, R^{B}, R^{C}, and R^{D} may be joined or fused to form a ring; and
wherein each of R^{A}, R^{B}, R^{C}, and R^{D} is independently hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof.

In another aspect, the present disclosure provides a formulation of the compound as described herein.

In yet another aspect, the present disclosure provides an OLED having an organic layer comprising the compound as described herein.

In yet another aspect, the present disclosure provides a consumer product comprising an OLED with an organic layer comprising the compound as described herein.

### BRIEF DESCRIPTION OF THE DRAWINGS

FIG. 1 shows an organic light emitting device.

FIG. 2 shows an inverted organic light emitting device that does not have a separate electron transport layer.

FIG. 3 shows a graph of modeled P-polarized photoluminescence as a function of angle for emitters with different vertical dipole ratio (VDR) values.

### DETAILED DESCRIPTION

### A. Terminology

Unless otherwise specified, the below terms used herein are defined as follows:

As used herein, “top” means furthest away from the substrate, while “bottom” means closest to the substrate. Where a first layer is described as “disposed over” a second layer, the first layer is disposed further away from substrate. There may be other layers between the first and second layer, unless it is specified that the first layer is “in contact with” the second layer. For example, a cathode may be described as “disposed over” an anode, even though there are various organic layers in between.

As used herein, “solution processable” means capable of being dissolved, dispersed, or transported in and/or deposited from a liquid medium, either in solution or suspension form.

As used herein, and as would be generally understood by one skilled in the art, a first “Highest Occupied Molecular Orbital” (HOMO) or “Lowest Unoccupied Molecular Orbital” (LUMO) energy level is “greater than” or “higher than” a second HOMO or LUMO energy level if the first energy level is closer to the vacuum energy level. Since ionization potentials (IP) are measured as a negative energy relative to a vacuum level, a higher HOMO energy level corresponds to an IP having a smaller absolute value (an IP that is less negative). Similarly, a higher LUMO energy level corresponds to an electron affinity (EA) having a smaller absolute value (an EA that is less negative). On a conventional energy level diagram, with the vacuum level at the top, the LUMO energy level of a material is higher than the HOMO energy level of the same material. A “higher” HOMO or LUMO energy level appears closer to the top of such a diagram than a “lower” HOMO or LUMO energy level.

As used herein, and as would be generally understood by one skilled in the art, a first work function is “greater than” or “higher than” a second work function if the first work function has a higher absolute value. Because work functions are generally measured as negative numbers relative to vacuum level, this means that a “higher” work function is more negative. On a conventional energy level diagram, with the vacuum level at the top, a “higher” work function is illustrated as further away from the vacuum level in the downward direction. Thus, the definitions of HOMO and LUMO energy levels follow a different convention than work functions.

The terms “halo,” “halogen,” and “halide” are used interchangeably and refer to fluorine, chlorine, bromine, and iodine.

The term “acyl” refers to a substituted carbonyl group (—C(O)—Rₛ).

The term “ester” refers to a substituted oxycarbonyl (—O—C(O)—Rₛ or —C(O)—O—Rₛ) group.

The term “ether” refers to an —OR, group.

The terms “sulfanyl” or “thio-ether” are used interchangeably and refer to a —SRₛ group.

The term “selenyl” refers to a —SeRₛ group.

The term “sulfinyl” refers to a —S(O)—Rₛ group.

The term “sulfonyl” refers to a —SO₂—Rₛ group.

The term “phosphino” refers to a group containing at least one phosphorus atom bonded to the relevant structure. Common examples of phosphino groups include, but are not limited to, groups such as a —P(Rₛ)₂ group or a —PO(Rₛ)₂ group, wherein each Rₛ can be same or different.

The term “silyl” refers to a group containing at least one silicon atom bonded to the relevant structure. Common examples of silyl groups include, but are not limited to, groups such as a —Si(Rₛ)₃ group, wherein each Rₛ can be same or different.

The term “germyl” refers to a group containing at least one germanium atom bonded to the relevant structure. Common examples of germyl groups include, but are not limited to, groups such as a —Ge(Rₛ)₃ group, wherein each Rₛ can be same or different.

The term “boryl” refers to a group containing at least one boron atom bonded to the relevant structure. Common examples of boryl groups include, but are not limited to, groups such as a —B(Rₛ)₂ group or its Lewis adduct —B(Rₛ)₃ group, wherein Rₛ can be same or different.

In each of the above, Rₛ can be hydrogen, or a substituent selected from the group consisting of the General Substituents as defined in this application. Preferred Rₛ is selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, and combination thereof. More preferably Rₛ is selected from the group consisting of alkyl, cycloalkyl, aryl, heteroaryl, and combination thereof.

The term “alkyl” refers to and includes both straight and branched chain alkyl groups having an alkyl carbon atom bonded to the relevant structure. Preferred alkyl groups are those containing from one to fifteen carbon atoms, preferably one to nine carbon atoms, and the preferred alkyl groups include methyl, ethyl, n-propyl, iso-propyl, n-butyl, sec-butyl, iso-butyl, tert-butyl, n-pentyl, 2-methylbutyl, 3-methylbutyl, 2,2-dimethylpropyl, 1,3-dimethylpropyl, 1,1-dimethylpropyl, 2-ethylpropyl, 1,2-dimethylpropyl, n-hexyl, 2-methylpentyl, 3-methylpentyl, 2,2-dimethylbutyl, 2,3-dimethylbutyl, n-heptyl, 2-methylhexyl, 3-methylhexyl, 2,2-dimethylpentyl, 2,3-dimethylpentyl, 2,4-dimethylpentyl, 3,3-dimethylpentyl, 3-ethylpentyl, 2,2,3-trimethylbutyl, and the like. Additionally, the alkyl group can be further substituted.

The term “cycloalkyl” refers to and includes monocyclic, polycyclic, and spiro alkyl groups having a ring alkyl carbon atom bonded to the relevant structure. Preferred cycloalkyl groups are those containing 3 to 12 ring carbon atoms and includes cyclopropyl, cyclopentyl, cyclohexyl, bicyclo[3.1.1]heptyl, spiro[4.5]decyl, spiro[5.5]undecyl, adamantyl, and the like. Additionally, the cycloalkyl group can be further substituted.

The terms “heteroalkyl” or “heterocycloalkyl” refer to an alkyl or a cycloalkyl group, respectively, having at least one carbon atom replaced by a heteroatom. Optionally the at least one heteroatom is selected from O, S, N, P, B, Si, Ge and Se, preferably, O, S or N. Additionally, the heteroalkyl or heterocycloalkyl group can be further substituted.

The term “alkenyl” refers to and includes both straight and branched chain alkene groups. Alkenyl groups are essentially alkyl groups that include at least one carbon-carbon double bond in the alkyl chain with one carbon atom from the carbon-carbon double bond that is bonded to the relevant structure. Cycloalkenyl groups are essentially cycloalkyl groups that include at least one carbon-carbon double bond in the cycloalkyl ring. The term “heteroalkenyl” as used herein refers to an alkenyl group having at least one carbon atom replaced by a heteroatom. Optionally the at least one heteroatom is selected from O, S, N, P, B, Si, Ge, and Se, preferably, O, S, or N. Preferred alkenyl, cycloalkenyl, or heteroalkenyl groups are those containing two to fifteen carbon atoms. Additionally, the alkenyl, cycloalkenyl, or heteroalkenyl group can be further substituted.

The term “alkynyl” refers to and includes both straight and branched chain alkyne groups. Alkynyl groups are essentially alkyl groups that include at least one carbon-carbon triple bond in the alkyl chain with one carbon atom from the carbon-carbon triple bond that is bonded to the relevant structure. Preferred alkynyl groups are those containing two to fifteen carbon atoms. Additionally, the alkynyl group can be further substituted.

The terms “aralkyl” or “arylalkyl” are used interchangeably and refer to an aryl-substituted alkyl group having an alkyl carbon atom bonded to the relevant structure. Additionally, the aralkyl group can be further substituted.

The term “heterocyclic group” refers to and includes aromatic and non-aromatic cyclic groups containing at least one heteroatom. Optionally the at least one heteroatom is selected from O, S, Se, N, P, B, Si, Ge, and Se, preferably, O, S, N, or B. Hetero-aromatic cyclic groups may be used interchangeably with heteroaryl. Preferred hetero-non-aromatic cyclic groups are those containing 3 to 10 ring atoms, preferably those containing 3 to 7 ring atoms, which includes at least one hetero atom, and includes cyclic amines such as morpholino, piperidino, pyrrolidino, and the like, and cyclic ethers/thio-ethers, such as tetrahydrofuran, tetrahydropyran, tetrahydrothiophene, and the like. Additionally, the heterocyclic group can be further substituted or fused.

The term “aryl” refers to and includes both single-ring and polycyclic aromatic hydrocarbyl groups. The polycyclic rings may have two or more rings in which two carbons are common to two adjoining rings (the rings are “fused”). Preferred aryl groups are those containing six to thirty carbon atoms, preferably six to twenty-four carbon atoms, six to eighteen carbon atoms, and more preferably six to twelve carbon atoms. Especially preferred is an aryl group having six carbons, ten carbons, twelve carbons, fourteen carbons, or eighteen carbons. Suitable aryl groups include phenyl, biphenyl, triphenyl, triphenylene, tetraphenylene, naphthalene, anthracene, phenalene, phenanthrene, pyrene, chrysene, perylene, and azulene, preferably phenyl, biphenyl, triphenyl, triphenylene, and naphthalene. Additionally, the aryl group can be further substituted or fused, such as, without limitation, fluorene.

The term “heteroaryl” refers to and includes both single-ring aromatic groups and polycyclic aromatic ring systems that include at least one heteroatom. The heteroatoms include, but are not limited to O, S, Se, N, P, B, Si, Ge, and Se. In many instances, O, S, N, or B are the preferred heteroatoms. Hetero-single ring aromatic systems are preferably single rings with 5 or 6 ring atoms, and the ring can have from one to six heteroatoms. The hetero-polycyclic ring systems can have two or more aromatic rings in which two atoms are common to two adjoining rings (the rings are “fused”) wherein at least one of the rings is a heteroaryl. The hetero-polycyclic aromatic ring systems can have from one to six heteroatoms per ring of the polycyclic aromatic ring system. Preferred heteroaryl groups are those containing three to thirty carbon atoms, preferably three to twenty-four carbon atoms, three to eighteen carbon atoms, and more preferably three to twelve carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, selenophenodipyridine, azaborine, borazine, 5λ²,9λ²-diaza-13b-boranaphtho[2,3,4-de]anthracene, 5λ²-benzo[d]benzo[4,5]imidazo[3,2-a]imidazole, and 5,9-dioxa-13b-boranaphtho[3,2,1-de]anthracene; preferably dibenzothiophene, dibenzofuran, dibenzoselenophene, carbazole, indolocarbazole, imidazole, pyridine, triazine, benzimidazole, 5λ²,9λ²-diaza-13b-boranaphtho[2,3,4-de]anthracene, 5λ²-benzo[d]benzo[4,5]imidazo[3,2-a]imidazole, and 5,9-dioxa-13b-boranaphtho[3,2,1-de]anthracene. Additionally, the heteroaryl group can be further substituted or fused.

Of the aryl and heteroaryl groups listed above, the groups of triphenylene, naphthalene, anthracene, dibenzothiophene, dibenzofuran, dibenzoselenophene, carbazole, indolocarbazole, imidazole, pyridine, pyrazine, pyrimidine, triazine, benzimidazole, 5λ²,9λ²-diaza-13b-boranaphtho[2,3,4-de]anthracene, 5λ²-benzo[d]benzo[4,5]imidazo[3,2-a]imidazole, 5,9-dioxa-13b-boranaphtho[3,2,1-de]anthracene, and the respective aza-analogs of each thereof are of particular interest.

In many instances, the General Substituents are selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, selenyl, sulfinyl, sulfonyl, phosphino, and combinations thereof.

In some instances, the Preferred General Substituents are selected from the group consisting of deuterium, fluorine, alkyl, cycloalkyl, heteroalkyl, alkoxy, aryloxy, amino, silyl, germyl, boryl, alkenyl, cycloalkenyl, heteroalkenyl, aryl, heteroaryl, nitrile, isonitrile, sulfanyl, and combinations thereof.

In some instances, the More Preferred General Substituents are selected from the group consisting of deuterium, fluorine, alkyl, cycloalkyl, alkoxy, aryloxy, amino, silyl, germyl, boryl, aryl, heteroaryl, nitrile, sulfanyl, and combinations thereof.

In some instances, the Even More Preferred General Substituents are selected from the group consisting of deuterium, fluorine, alkyl, cycloalkyl, silyl, aryl, heteroaryl, nitrile, and combinations thereof.

In yet other instances, the Most Preferred General Substituents are selected from the group consisting of deuterium, alkyl, cycloalkyl, aryl, heteroaryl, and combinations thereof.

In the event one or more substituents (e.g., R, R′, R″, R^{A}, R_{A}, R¹, R₁, etc.) is not specifically defined, each of the one or more substituents shall be understood to independently represent hydrogen or a substituent selected from the group consisting of the General Substituents defined herein. Similarly, each of the one or more substituents can optionally be joined or fused with another substituent to form a ring. It shall also be understood that any substituent that can be selected from the General Substituents defined herein can also be selected from the Preferred General Substituents defined herein, the More Preferred General Substituents defined herein, the Even More Preferred General Substituents defined herein, or the Most Preferred General Substituents defined herein.

The terms “substituted” and “substitution” refer to a substituent other than H that is bonded to the relevant position, e.g., a carbon or nitrogen. For example, when R¹ represents mono-substitution, then one R¹ must be other than H (i.e., a substitution). Similarly, when R¹ represents di-substitution, then two of R¹ must be other than H. Similarly, when R¹ represents zero or no substitution, R¹, for example, can be a hydrogen for all available valencies of ring atoms, as in carbon atoms for benzene and the nitrogen atom in pyrrole, or simply represents nothing for ring atoms with fully filled valencies, e.g., the nitrogen atom in pyridine. The maximum number of substitutions possible in a ring structure will depend on the total number of available valencies in the ring atoms.

As used herein, “combinations thereof” indicates that one or more members of the applicable list are combined to form a known or chemically stable arrangement that one of ordinary skill in the art can envision from the applicable list. For example, an alkyl and deuterium can be combined to form a partial or fully deuterated alkyl group; a halogen and alkyl can be combined to form a halogenated alkyl substituent; and a halogen, alkyl, and aryl can be combined to form a halogenated arylalkyl. In one instance, the term substitution includes a combination of two to four of the listed groups. In another instance, the term substitution includes a combination of two to three groups. In yet another instance, the term substitution includes a combination of two groups. Preferred combinations of substituent groups are those that contain up to fifty atoms that are not hydrogen or deuterium, or those which include up to forty atoms that are not hydrogen or deuterium, or those that include up to thirty atoms that are not hydrogen or deuterium. In many instances, a preferred combination of substituent groups will include up to twenty atoms that are not hydrogen or deuterium.

The “aza” designation in the fragments described herein, i.e. aza-dibenzofuran, aza-dibenzothiophene, etc. means that one or more of the C—H groups in the respective aromatic ring can be replaced by a nitrogen atom, for example, and without any limitation, azatriphenylene encompasses both dibenzo[fh]quinoxaline and dibenzo[fh]quinoline. One of ordinary skill in the art can readily envision other nitrogen analogs of the aza-derivatives described above, and all such analogs are intended to be encompassed by the terms as set forth herein.

The present disclosure includes all acceptable isotopically-labelled compounds of the present disclosure wherein one or more atoms are replaced by atoms having the same atomic number, but an atomic mass or mass number different from the atomic mass or mass number usually found in nature.

Examples of isotopes suitable for inclusion in the compounds of the present disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I, ¹²⁴I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of the present disclosure, for example, those incorporating a radioactive isotope, are useful in diagnostic and other studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain advantages resulting from greater stability, and hence may be preferred in some circumstances.

Isotopically-labelled compounds of the invention can generally be prepared by conventional techniques known to those skilled in the art or by processes analogous to those described in the accompanying Examples and Preparations using an appropriate isotopically-labelled reagents in place of the non-labelled reagent previously employed.

For examples, deuterated compounds can be readily prepared using methods known in the art. For example, U.S. Pat. No. 8,557,400, Patent Pub. No. WO 2006/095951, and U.S. Pat. Application Pub. No. US 2011/0037057, which are hereby incorporated by reference in their entireties, describe the making of deuterium-substituted organometallic complexes. Further reference is made to Ming Yan, et al., Tetrahedron 2015, 71, 1425-30 and Atzrodt et al., Angew. Chem. Int. Ed. (Reviews) 2007, 46, 7744-65, which are incorporated by reference in their entireties, describe the deuteration of the methylene hydrogens in benzyl amines and efficient pathways to replace aromatic ring hydrogens with deuterium, respectively.

As used herein, any specifically listed substituent, such as, without limitation, methyl, phenyl, pyridyl, etc. includes undeuterated, partially deuterated, and fully deuterated versions thereof. Similarly, classes of substituents such as, without limitation, alkyl, aryl, cycloalkyl, heteroaryl, etc. also include undeuterated, partially deuterated, and fully deuterated versions thereof. Unless otherwise specified, atoms in chemical structures without valences fully filled by H or D should be considered to include undeuterated, partially deuterated, and fully deuterated versions thereof. For example, the chemical structure of

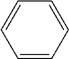

implies to include C₆H₆, C₆D₆, C₆H₃D₃, and any other partially deuterated variants thereof. Some common basic partially or fully deuterated groups include, without limitation, CD₃, CD₂C(CH₃)₃, C(CD₃)₃, and C₆D₅. Similarly, where partially or fully defined atomic structures show a particular position may be or is deuterium, the same atomic structures with one, two, or up to all deuterium atoms replaced by hydrogen are also envisioned.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or attached fragment are considered to be equivalent.

In some instances, a pair of substituents in the molecule can be joined or fused into a ring. The preferred ring is a five to nine-membered carbocyclic or heterocyclic ring, includes both instances where the portion of the ring formed by the pair of substituents is saturated and where the portion of the ring formed by the pair of substituents is unsaturated. In yet other instances, a pair of adjacent substituents can be joined or fused into a ring. As used herein, “adjacent” means that the two substituents involved can be on the same ring next to each other, or on two neighboring rings having the two closest available substitutable positions, such as 2, 2′ positions in a biphenyl, or 1, 8 position in a naphthalene.

Layers, materials, regions, and devices may be described herein in reference to the color of light they emit. In general, as used herein, an emissive region that is described as producing a specific color of light may include one or more emissive layers disposed over each other in a stack.

As used herein, a “NIR”, “red”, “green”, “blue”, “yellow” layer, material, region, or device refers to a layer, a material, a region, or a device that emits light in the wavelength range of about 700-1500 nm, 580-700 nm, 500-600 nm, 400-500 nm, 540-600 nm, respectively, or a layer, a material, a region, or a device that has a highest peak in its emission spectrum in the respective wavelength region. In some arrangements, separate regions, layers, materials, or devices may provide separate “deep blue” and “light blue” emissions. As used herein, the “deep blue” emission component refers to an emission having a peak emission wavelength that is at least about 4 nm less than the peak emission wavelength of the “light blue” emission component. Typically, a “light blue” emission component has a peak emission wavelength in the range of about 465-500 nm, and a “deep blue” emission component has a peak emission wavelength in the range of about 400-470 nm, though these ranges may vary for some configurations.

In some arrangements, a color altering layer that converts, modifies, or shifts the color of the light emitted by another layer to an emission having a different wavelength is provided. Such a color altering layer can be formulated to shift wavelength of the light emitted by the other layer by a defined amount, as measured by the difference in the wavelength of the emitted light and the wavelength of the resulting light. In general, there are two classes of color altering layers: color filters that modify a spectrum by removing light of unwanted wavelengths, and color changing layers that convert photons of higher energy to lower energy. For example, a “red” color filter can be present in order to filter an input light to remove light having a wavelength outside the range of about 580-700 nm. A component “of a color” refers to a component that, when activated or used, produces or otherwise emits light having a particular color as previously described. For example, a “first emissive region of a first color” and a “second emissive region of a second color different than the first color” describes two emissive regions that, when activated within a device, emit two different colors as previously described.

As used herein, emissive materials, layers, and regions may be distinguished from one another and from other structures based upon light initially generated by the material, layer or region, as opposed to light eventually emitted by the same or a different structure. The initial light generation typically is the result of an energy level change resulting in emission of a photon. For example, an organic emissive material may initially generate blue light, which may be converted by a color filter, quantum dot or other structure to red or green light, such that a complete emissive stack or sub-pixel emits the red or green light. In this case the initial emissive material or layer may be referred to as a “blue” component, even though the sub-pixel is a “red” or “green” component.

In some cases, it may be preferable to describe the color of a component such as an emissive region, sub-pixel, color altering layer, or the like, in terms of 1931 CIE coordinates. For example, a yellow emissive material may have multiple peak emission wavelengths, one in or near an edge of the “green” region, and one within or near an edge of the “red” region as previously described. Accordingly, as used herein, each color term also corresponds to a shape in the 1931 CIE coordinate color space. The shape in 1931 CIE color space is constructed by following the locus between two color points and any additional interior points. For example, interior shape parameters for red, green, blue, and yellow may be defined as shown below:

| Color | CIE Shape Parameters |
|---|---|
| Central Red | Locus: [0.6270, 0.3725]; [0.7347, 0.2653]; |
| | Interior: [0.5086, 0.2657] |
| Central Green | Locus: [0.0326, 0.3530]; [0.3731, 0.6245]; |
| | Interior: [0.2268, 0.3321 |
| Central Blue | Locus: [0.1746, 0.0052]; [0.0326, 0.3530]; |
| | Interior: [0.2268, 0.3321] |
| Central Yellow | Locus: [0.3731, 0.6245]; [0.6270, 0.3725]; |
| | Interior: [0.3700, 0.4087]; [0.2886, 0.4572] |

More details on OLEDs, and the definitions described above, can be found in U.S. Pat. No. 7,279,704, which is incorporated herein by reference in its entirety.

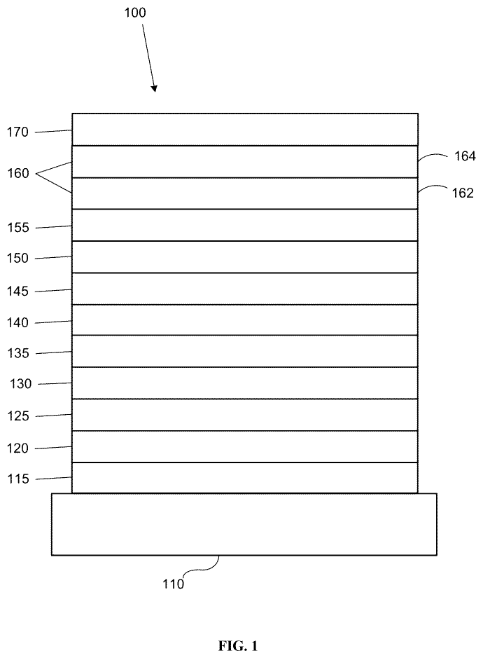

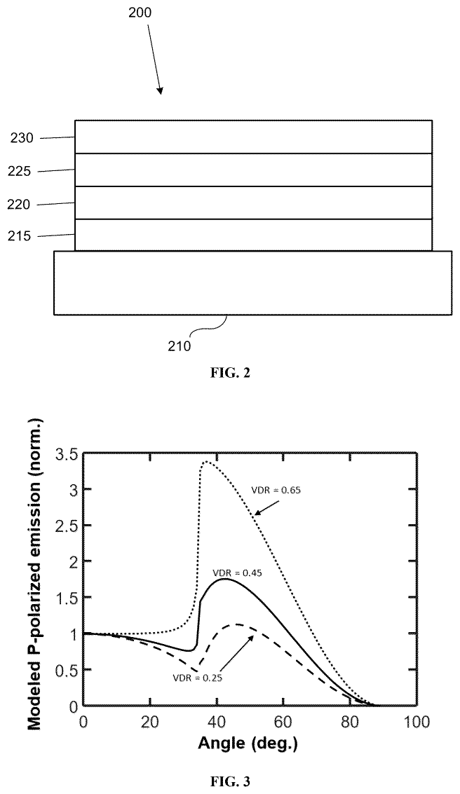

As disclosed herein, emissive layers or materials, such as emissive layer 135 and emissive layer 220 shown in FIGS. 1-2, respectively, may include quantum dots. An “emissive layer” or “emissive material” as disclosed herein may include an organic emissive material and/or an emissive material that contains quantum dots or equivalent structures, unless indicated to the contrary explicitly or by context according to the understanding of one of skill in the art. In general, an emissive layer includes emissive material within a host matrix. Such an emissive layer may include only a quantum dot material which converts light emitted by a separate emissive material or other emitter, or it may also include the separate emissive material or other emitter, or it may emit light itself directly from the application of an electric current. Similarly, a color altering layer, color filter, upconversion, or downconversion layer or structure may include a material containing quantum dots, though such layer may not be considered an “emissive layer” as disclosed herein. In general, an “emissive layer” or material is one that emits an initial light based on an injected electrical charge, where the initial light may be altered by another layer such as a color filter or other color altering layer that does not itself emit an initial light within the device, but may re-emit altered light of a different spectra content based upon absorption of the initial light emitted by the emissive layer and downconversion to a lower energy light emission. In some embodiments disclosed herein, the color altering layer, color filter, upconversion, and/or downconversion layer may be disposed outside of an OLED device, such as above or below an electrode of the OLED device.

Unless otherwise specified, any of the layers of the various embodiments may be deposited by any suitable method. For the organic layers, preferred methods include thermal evaporation, ink-jet, such as described in U.S. Pat. Nos. 6,013,982 and 6,087,196, which are incorporated by reference in their entireties, organic vapor phase deposition (OVPD), such as described in U.S. Pat. No. 6,337,102 to Forrest et al., which is incorporated by reference in its entirety, and deposition by organic vapor jet printing (OVJP, also referred to as organic vapor jet deposition (OVJD)), such as described in U.S. Pat. No. 7,431,968, which is incorporated by reference in its entirety. Other suitable deposition methods include spin coating and other solution based processes. Solution based processes are preferably carried out in nitrogen or an inert atmosphere. For the other layers, preferred methods include thermal evaporation. Preferred patterning methods include deposition through a mask, cold welding such as described in U.S. Pat. Nos. 6,294,398 and 6,468,819, which are incorporated by reference in their entireties, and patterning associated with some of the deposition methods such as ink-jet and OVJD. Other methods may also be used. The materials to be deposited may be modified to make them compatible with a particular deposition method. For example, substituents such as alkyl and aryl groups, branched or unbranched, and preferably containing at least 3 carbons, may be used in small molecules to enhance their ability to undergo solution processing. Substituents having 20 carbons or more may be used, and 3-20 carbons is a preferred range. Materials with asymmetric structures may have better solution processibility than those having symmetric structures, because asymmetric materials may have a lower tendency to recrystallize. Dendrimer substituents may be used to enhance the ability of small molecules to undergo solution processing.

Devices fabricated in accordance with embodiments of the present disclosure may further optionally comprise a barrier layer. One purpose of the barrier layer is to protect the electrodes and organic layers from damaging exposure to harmful species in the environment including moisture, vapor and/or gases, etc. The barrier layer may be deposited over, under or next to a substrate, an electrode, or over any other parts of a device including an edge. The barrier layer may comprise a single layer, or multiple layers. The barrier layer may be formed by various known chemical vapor deposition techniques and may include compositions having a single phase as well as compositions having multiple phases. Any suitable material or combination of materials may be used for the barrier layer. The barrier layer may incorporate an inorganic or an organic compound or both. The preferred barrier layer comprises a plurality of alternative layers of polymeric material and non-polymeric material; organic material and inorganic material; or a mixture of a polymeric material and a non-polymeric material as one example described in U.S. Pat. No. 7,968,146, PCT Pat. Application Nos. PCT/US2007/023098 and PCT/US2009/042829, which are herein incorporated by reference in their entireties.

Devices fabricated in accordance with embodiments of the disclosure can be incorporated into a wide variety of electronic component modules (or units) that can be incorporated into a variety of electronic products or intermediate components. Examples of such electronic products or intermediate components include display screens, lighting devices such as discrete light source devices or lighting panels, etc. that can be utilized by the end-user product manufacturers. Such electronic component modules can optionally include the driving electronics and/or power source(s). Devices fabricated in accordance with embodiments of the disclosure can be incorporated into a wide variety of consumer products that have one or more of the electronic component modules (or units) incorporated therein. A consumer product comprising an OLED that includes the compound of the present disclosure in the organic layer in the OLED is disclosed. Such consumer products would include any kind of products that include one or more light source(s) and/or one or more of some type of visual displays. Some examples of such consumer products include a flat panel display, a curved display, a computer monitor, a medical monitor, a television, a billboard, a light for interior or exterior illumination and/or signaling, a heads-up display, a fully or partially transparent display, a flexible display, a rollable display, a foldable display, a stretchable display, a laser printer, a telephone, a cell phone, tablet, a phablet, a personal digital assistant (PDA), a wearable device, a laptop computer, a digital camera, a camcorder, a viewfinder, a micro-display that is less than 2 inches diagonal, a 3-D display, a virtual reality or augmented reality display, a vehicle, a video walls comprising multiple displays tiled together, a theater or stadium screen, and a sign. Various control mechanisms may be used to control devices fabricated in accordance with the present disclosure, including passive matrix and active matrix. Many of the devices are intended for use in a temperature range comfortable to humans, such as 18 C to 30 C, and more preferably at room temperature (20-25 C), but could be used outside this temperature range, for example, from −40 C to 80 C.

The materials and structures described herein may have applications in devices other than OLEDs. For example, other optoelectronic devices such as organic solar cells and organic photodetectors may employ the materials and structures. More generally, organic devices, such as organic transistors, may employ the materials and structures.

In general parlance in the art, a “sub-pixel” may refer to the emissive region, which may be a single-layer EML, a stacked device, or the like, in conjunction with any color altering layer that is used to modify the color emitted by the emissive region.

As used herein, the “emissive region” of a sub-pixel refers to any and all emissive layers, regions, and devices that are used initially to generate light for the sub-pixel. A sub-pixel also may include additional layers disposed in a stack with the emissive region that affect the color ultimately produced by the sub-pixel, such as color altering layers disclosed herein, though such color altering layers typically are not considered “emissive layers” as disclosed herein. An unfiltered sub-pixel is one that excludes a color modifying component such as a color altering layer, but may include one or more emissive regions, layers, or devices.

In some configurations, an “emissive region” may include emissive materials that emit light of multiple colors. For example, a yellow emissive region may include multiple materials that emit red and green light when each material is used in an OLED device alone. When used in a yellow device, the individual materials typically are not arranged such that they can be individually activated or addressed. That is, the “yellow” OLED stack containing the materials cannot be driven to produce red, green, or yellow light; rather, the stack can be driven as a whole to produce yellow light. Such an emissive region may be referred to as a yellow emissive region even though, at the level of individual emitters, the stack does not directly produce yellow light. As described in further detail below, the individual emissive materials used in an emissive region (if more than one), may be placed in the same emissive layer within the device, or in multiple emissive layers within an OLED device comprising an emissive region. As described in further detail below, embodiments disclosed herein may allow for OLED devices such as displays that include a limited number of colors of emissive regions, while including more colors of sub-pixels or other OLED devices than the number of colors of emissive regions. For example, a device as disclosed herein may include only blue and yellow emissive regions. Additional colors of sub-pixels may be achieved by the use of color altering layers, such as color altering layers disposed in a stack with yellow or blue emissive regions, or more generally through the use of color altering layers, electrodes or other structures that form a microcavity as disclosed herein, or any other suitable configuration. In some cases, the general color provided by a sub-pixel may be the same as the color provided by the emissive region in the stack that defines the sub-pixel, such as where a deep blue color altering layer is disposed in a stack with a light blue emissive region to produce a deep blue sub-pixel. Similarly, the color provided by a sub-pixel may be different than the color provided by an emissive region in the stack that defines the sub-pixel, such as where a green color altering layer is disposed in a stack with a yellow emissive region to product a green sub-pixel.

In some configurations, emissive regions and/or emissive layers may span multiple sub-pixels, such as where additional layers and circuitry are fabricated to allow portions of an emissive region or layer to be separately addressable.

An emissive region as disclosed herein may be distinguished from an emissive “layer” as typically referred to in the art and as used herein. In some cases, a single emissive region may include multiple layers, such as where a yellow emissive region is fabricated by sequentially deposited red and green emissive layers to form the yellow emissive region. As previously described, when such layers occur in an emissive region as disclosed herein, the layers are not individually addressable within a single emissive stack; rather, the layers are activated or driven concurrently to produce the desired color of light for the emissive region. In other configurations, an emissive region may include a single emissive layer of a single color, or multiple emissive layers of the same color, in which case the color of such an emissive layer will be the same as, or in the same region of the spectrum as, the color of the emissive region in which the emissive layer is disposed.

### B. The Compounds of the Present Disclosure

In one aspect, the present disclosure provides a compound of Formula I:

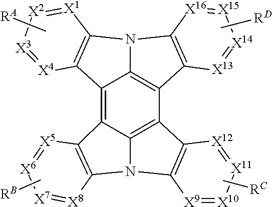

wherein each of X¹ to X¹⁶ is independently C or N;
each of R^{A}, R^{B}, R^{C}, and R^{D} independently represents mono to the maximum allowable substitutions, or no substitutions;
wherein any two of R^{A}, R^{B}, R^{C}, and R^{D} may be joined or fused to forma ring; and
wherein each of R^{A}, R^{B}, R^{C}, and R^{D} is independently hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof.

In some embodiments, if the compound comprises a structure of Formula II:

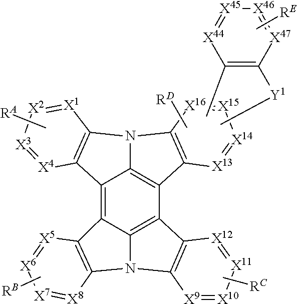

wherein each of X⁴⁴ to X⁴⁷ is independently C or N;
wherein Y¹ is selected from the group consisting of O, NR^{Y1}, and CMe₂;
wherein R^{E} represents mono to the maximum allowable substitutions, or no substitutions;
wherein each of R^{Y1} and R^{E} is independently hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof
then
(i) when Y¹ is O or CMe₂, then X¹⁵ is C, and (ii) when Y¹ is NR^{Y1}, then R^{Y1} and R^{E} do not join to form a 5-membered ring.

In some embodiments, if no two of R^{A}, two of R^{B}, two of R^{C}, or two of R^{D} are joined to form a ring, then at least one of R^{A}, R^{B}, R^{C}, or R^{D} comprises a structure selected from the group consisting of deuterium, alkyldiarylsilyl, dialkylarylsilyl, germyl, tetraphenylene, a bridged polycyclic ring, an alkyl or cycloalkyl comprising five or more carbons, and an aromatic ring fused by a non-aromatic ring;
with the proviso that, if two of R^{A}, two of R^{B}, two of R^{C}, or two of R^{D} are joined to form a benzene ring, then at least one of R^{A}, R^{B}, R^{C}, or R^{D} comprises a structure selected from the group consisting of deuterium, silyl, germyl, tetraphenylene, benzo[d]benzo[4,5]imidazo[1,2-a]imidazole (bimbim), a bridged polycyclic ring, an alkyl or cycloalkyl comprising five or more carbons, an aromatic ring fused by a non-aromatic ring, Formula III, and Formula IV,

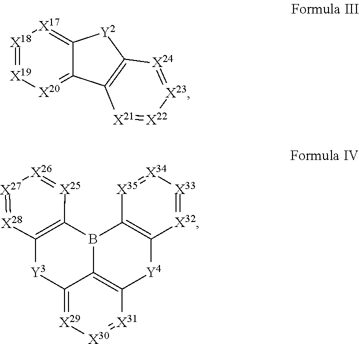

wherein each of X¹⁷ to X³⁵ is independently C or N, and each Y², Y³, and Y⁴ is independently selected from the group consisting of O, S, Se, NR^{Y2}, BR^{Y2}, BR^{Y2}R^{Y3}, CR^{Y2}R^{Y3}, SiR^{Y2}R^{Y3}, GeR^{Y2}R^{Y3}, C═O, C═S, C═NR^{Y2}, C═CR^{Y2}R^{Y3}, SO, SO₂, or P(O)R^{Y2};
wherein each of R^{Y2} and R^{Y3} is independently hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof.

In some embodiments, if two of R^{A}, two of R^{B}, two of R^{C}, or two of R^{D} are joined to form an 5 or 6-membered cycloalkyl ring, then at least one of R^{A}, R^{B}, R^{C}, or R^{D} comprises a structure selected from the group consisting of deuterium, alkyldiarylsilyl, dialkylarylsilyl, triarylsilane, substituted triphenylsilyl, germyl, tetraphenylene, benzo[d]benzo[4,5]imidazo[1,2-a]imidazole, a bridged polycyclic ring, an alkyl or cycloalkyl comprising five or more carbons, an aromatic ring fused by a non-aromatic ring, and Formula V:

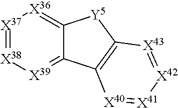

wherein each of X³⁶ to X⁴³ is independently C or N, and
wherein Y⁵ is selected from the group consisting of O, S, Se, NR^{Y4}, BR^{Y4}, BR^{Y4}R^{Y5}, CR^{Y4}R^{Y5}, SiR^{Y4}R^{Y5}, GeR^{Y4}R^{Y5}, C═O, C═S, C═NR^{Y4}, C═CR^{Y4}R^{Y5}, SO, SO₂, or P(O)R^{Y4};
wherein each of R^{Y4} and R^{Y5} is independently hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof.

In some embodiments, the compound is not:

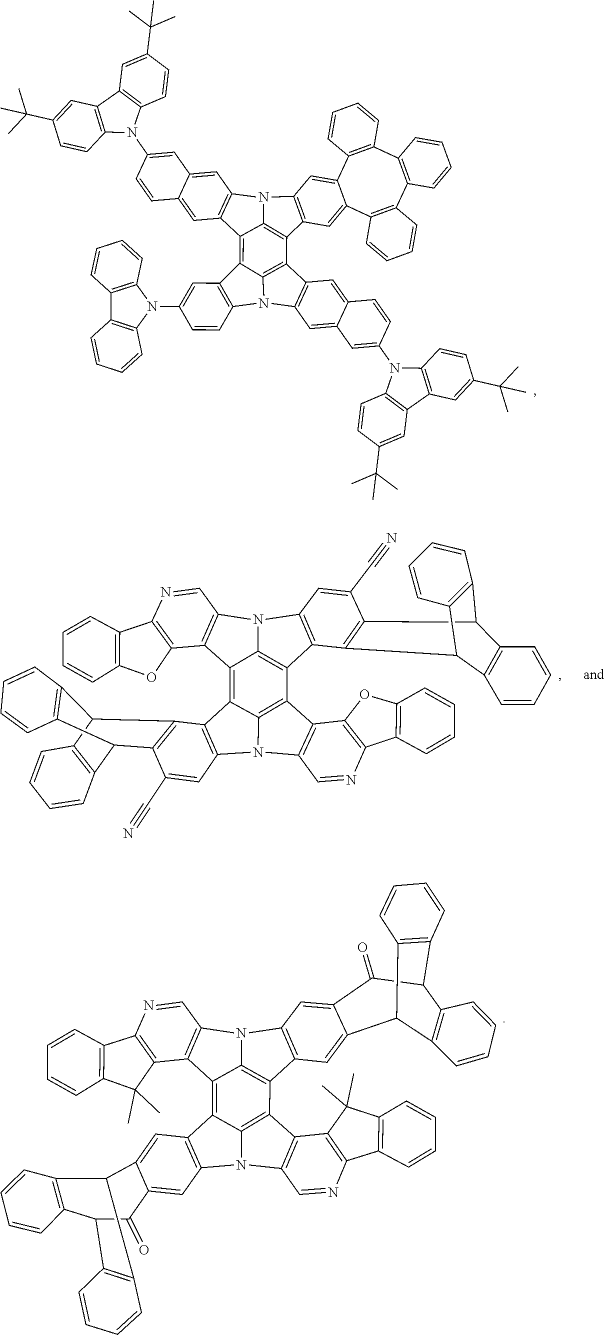

In some embodiments of Formula II, while

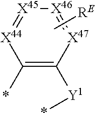

is showing to be fused to ring of X¹³-X¹⁶, it is understood that it can be fused to ring of X¹-X⁴, or ring of X⁵-X⁸, or ring of X⁹-X¹² respectively.

In some embodiments, the compound consists essentially of Formula I.

In some embodiments, the compound has a structure of Formula I.

In some embodiments, any two substituents can be fused or joined to form a ring. In some embodiments, at least two of R^{A}, R^{B}, R^{C}, or R^{D} are alkyl and joined to form into a ring, which can be further substituted. In some embodiments, at least one R^{A} with one R^{B} are joined to form into a ring. In some embodiments, at least one R^{A} with one R^{D} are joined to form into a ring. In some embodiments, at least one R^{B} with one R^{C} are joined to form into a ring. In some embodiments, at least one R^{C} with one R^{D} are joined to form into a ring. In some of the above embodiments, the ring formed by the two substituents can be an aromatic ring or a non-aromatic ring. In some of such embodiments, the non-aromatic ring can be formed by two alkyl substituents.

In some embodiments, at least one of R^{Y1}, R^{Y2}, R^{Y3}, R^{Y4}, R^{Y5}, R^{A}, R^{B}, R^{C}, R^{D}, and R^{E} is partially or fully deuterated. In some embodiments of Formula I, at least one of R^{Y1}, R^{Y2}, R^{Y3}, R^{Y4}, R^{Y5}, R^{A}, R^{B}, R^{C}, R^{D}, and R^{E} is selected from the general substituents described herein. In some embodiments of Formula I, at least one of R^{Y1}, R^{Y2}, R^{Y3}, R^{Y4}, R^{Y5}, R^{A}, R^{B}, R^{C}, R^{D}, and R^{E} is selected from the preferred general substituents described herein.

In some embodiments, the compound is fully or partially deuterated.

In some embodiments, the compound is fully deuterated.

In some embodiments, each of R^{Y1}, R^{Y2}, R^{Y3}, R^{Y4}, R^{Y5}, R^{A}, R^{B}, R^{C}, R^{D}, and R^{E} is independently a hydrogen or a substituent selected from the group consisting of deuterium, fluorine, alkyl, cycloalkyl, heteroalkyl, alkoxy, aryloxy, amino, silyl, germyl, boryl, alkenyl, cycloalkenyl, heteroalkenyl, aryl, heteroaryl, nitrile, isonitrile, sulfanyl, and combinations thereof.

In some embodiments, at least one of X¹ to X¹⁶ is N.

In some embodiments, at least one of X¹ to X⁴ is N.

In some embodiments, at least one of X⁵ to X⁸ is N.

In some embodiments, at least one of X⁹ to X¹² is N.

In some embodiments, at least one of X¹³ to X¹⁶ is N.

In some embodiments, all of X¹ to X¹⁶ are C.

In some embodiments, all R^{A} are hydrogen.

In some embodiments, all R^{B} are hydrogen.

In some embodiments, all R^{C} are hydrogen.

In some embodiments, all R^{D} are hydrogen.

In some embodiments, all R^{A} and R^{B} are hydrogen.

In some embodiments, all R^{A} and R^{C} are hydrogen.

In some embodiments, all R^{A} and R^{D} are hydrogen.

In some embodiments, all R^{A}, R^{B}, and R^{D} are hydrogen.

In some embodiments, all R^{A}, R^{C}, and R^{D} are hydrogen.

In some embodiments, the compound comprises a structure of Formula II.

In some embodiments, the compound has a structure of Formula II and is selected from the group consisting of the following structures (LIST 1a): ,

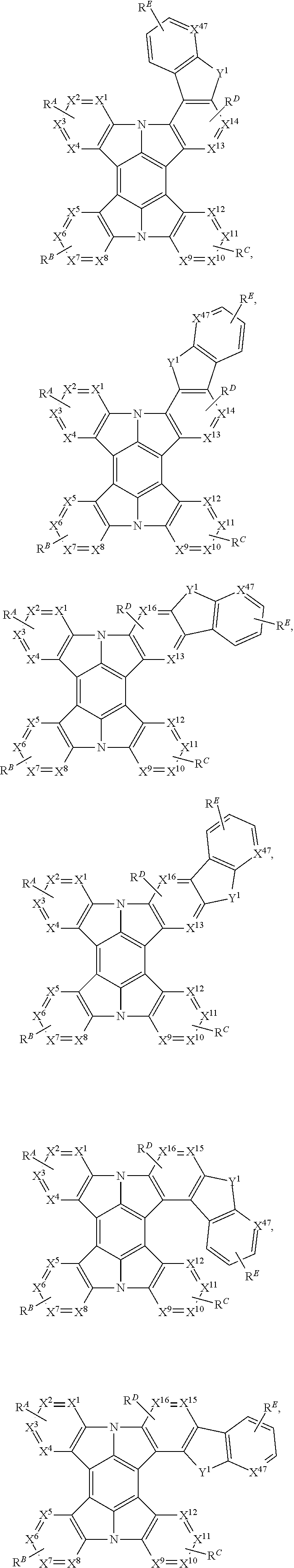

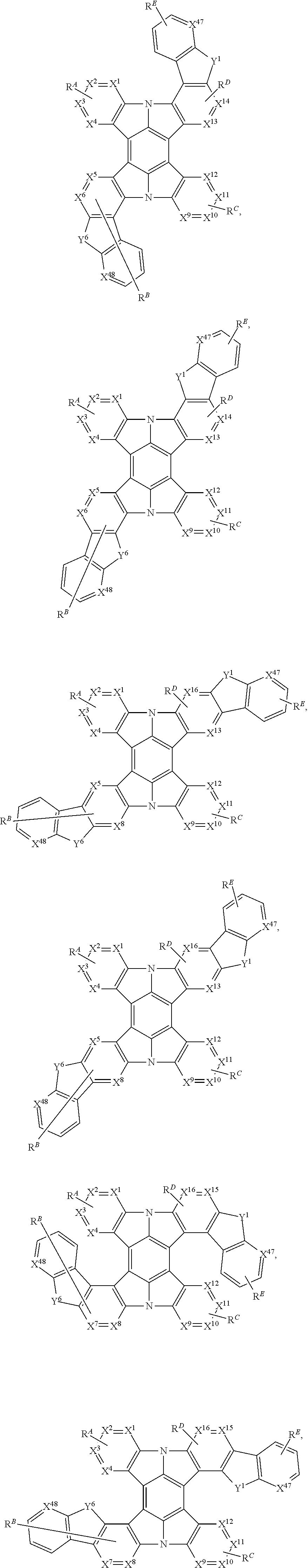

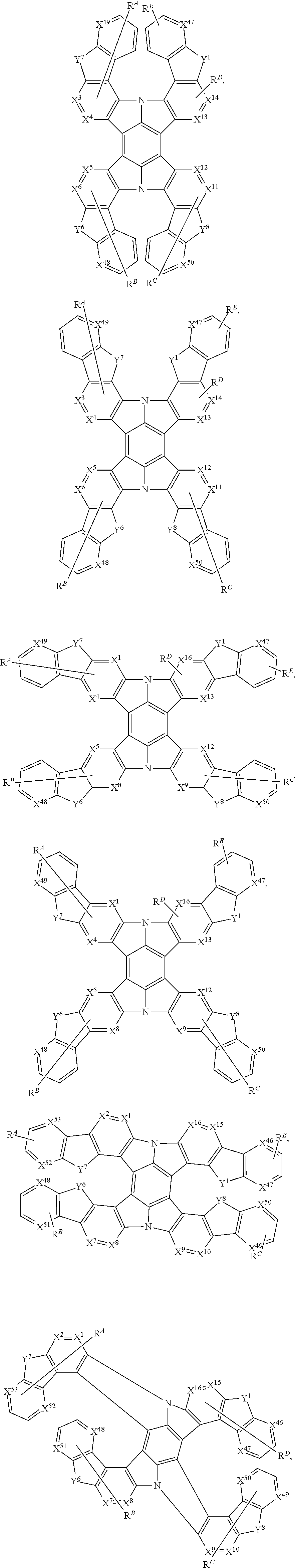

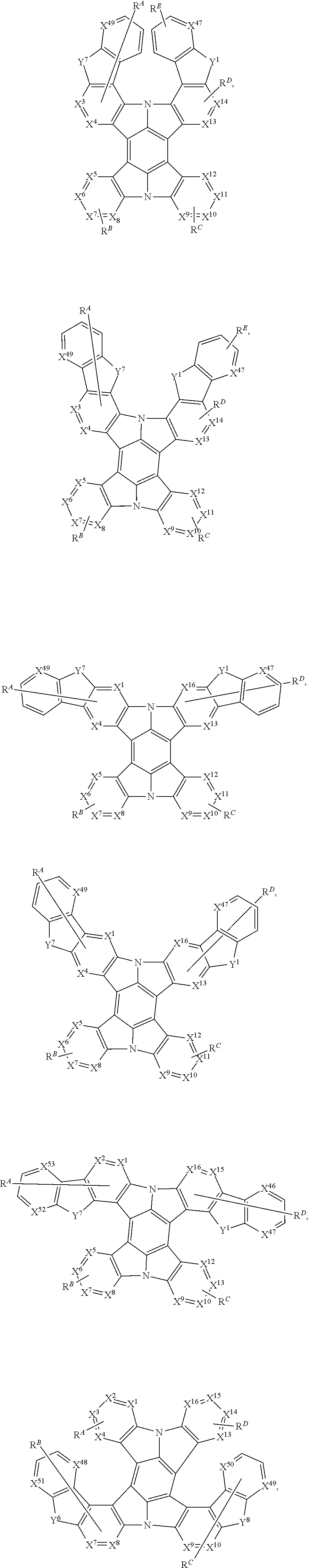

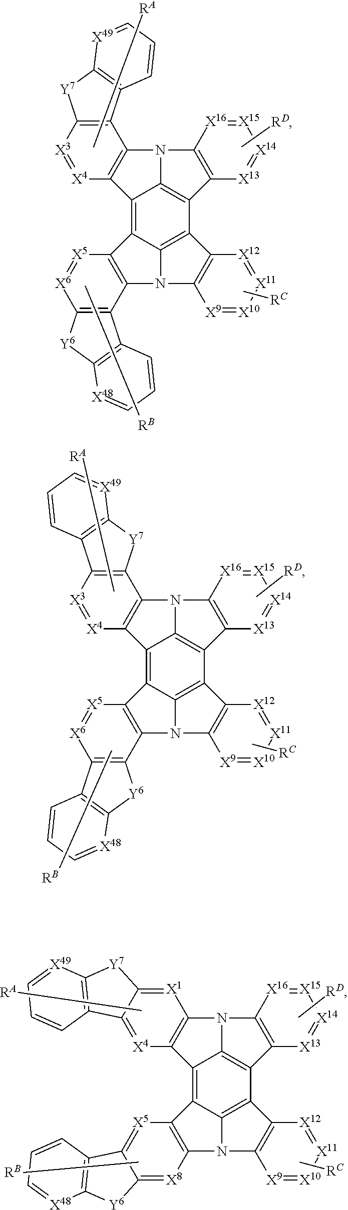

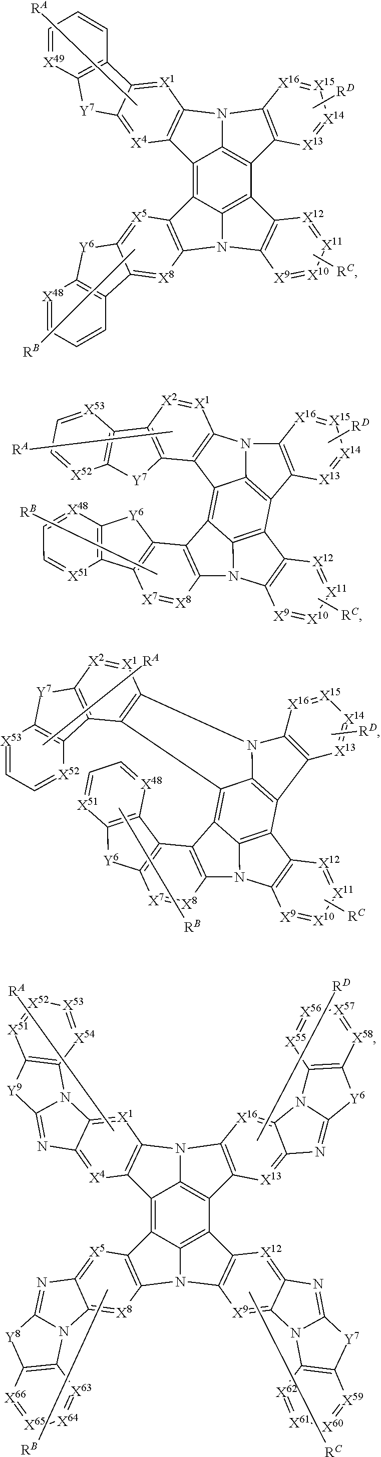

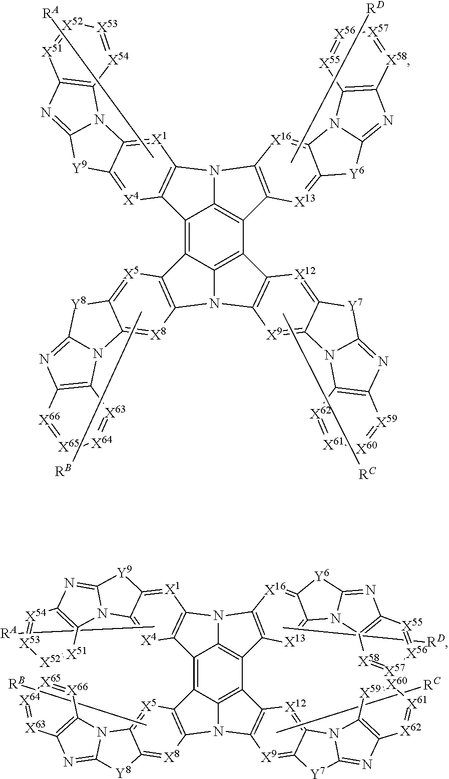

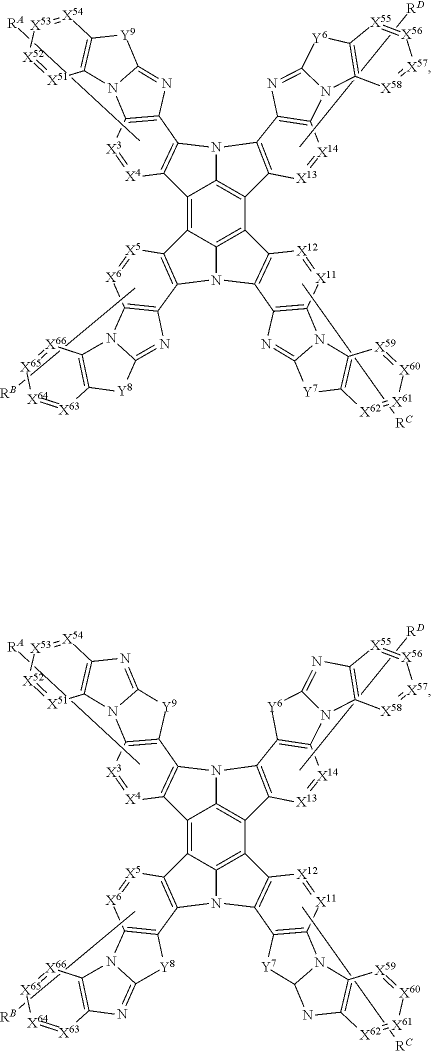

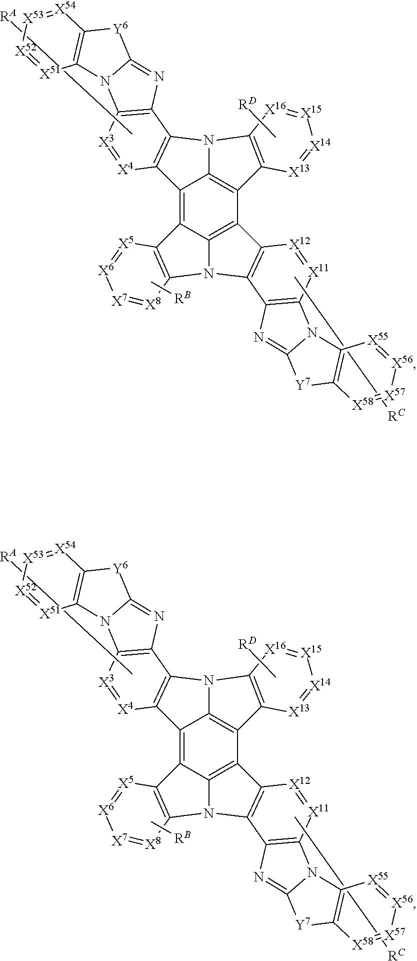

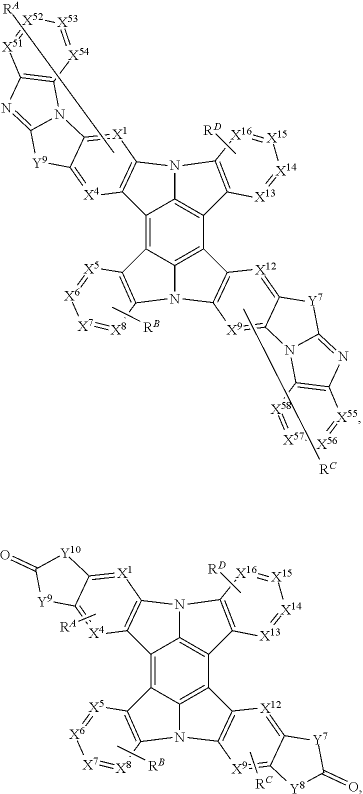

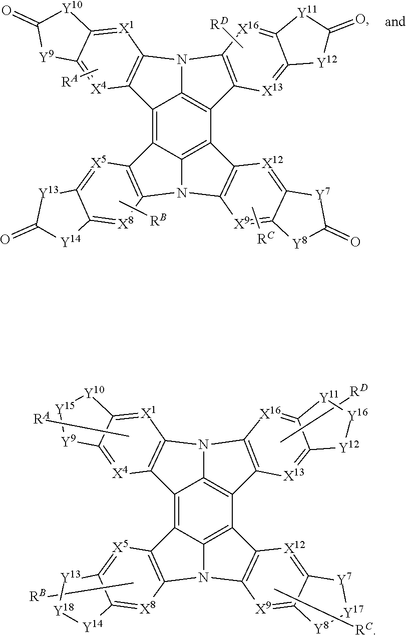

wherein Y¹ to Y¹⁸ is independently selected from the group consisting of O, S, Se, NR^{Y*}, BR^{Y*}, BR^{Y*}R^{Y*}, CR^{Y*}R^{Y*}, SiR^{Y*}R^{Y*′}, GeR^{Y*}R^{Y*′}, C═O, C═S, C═NR^{Y*}, C═CR^{Y*}R^{Y*′}, SO, SO₂, or P(O)R^{Y*};
wherein R^{Y*} and R^{Y*} is independently hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof; and
wherein X⁴⁸ to X⁶⁶ is independently C or N.

In some embodiments, X¹ to X⁶⁶ are all C.

In some embodiments, any of Y¹—Y¹⁴ is 0. In some embodiments, any of Y¹—Y¹⁴ is NR. In some embodiments, any of Y¹—Y¹⁴ is CRR′. In some embodiments, Y¹⁵—Y¹⁸ is BR.

In some embodiments, no two of R^{A}, two of R^{B}, two of R′, or two of R^{D} are joined to forma ring, and at least one of R^{A}, R^{B}, R^{C}, or R^{D} comprises a structure selected from the group consisting of deuterium, alkyldiarylsilyl, dialkylarylsilyl, germyl, tetraphenylene, a bridged polycyclic ring, an alkyl or cycloalkyl comprising five or more carbons, and an aromatic ring fused by a non-aromatic ring.

In some embodiments, X², X³, X⁶, X⁷, X¹⁰, X¹¹, X¹⁴ and X¹⁵ are all C.

In some embodiments, no two of R^{A}, two of R^{B}, two of R^{C}, or two of R^{D} are joined, and at least one of R^{A}, R^{B}, R^{C}, or R^{D} comprises a deuterium.

In some embodiments, no two of R^{A}, two of R^{B}, two of R^{C}, or two of R^{D} are joined, and at least one of R^{A}, R^{B}, R^{C}, or R^{D} comprises an alkyldiarylsilyl.

In some embodiments, no two of R^{A}, two of R^{B}, two of R^{C}, or two of R^{D} are joined, and at least one of R^{A}, R^{B}, R^{C}, or R^{D} comprises a dialkylarylsilyl.

In some embodiments, no two of R^{A}, two of R^{B}, two of R^{C}, or two of R^{D} are joined, and at least one of R^{A}, R^{B}, R^{C}, or R^{D} comprises a tetraphenylene.

In some embodiments, no two of R^{A}, two of R^{B}, two of R^{C}, or two of R^{D} are joined, and at least one of R^{A}, R^{B}, R^{C}, or R^{D} comprises a bicyclic alkyl.

In some embodiments, no two of R^{A}, two of R^{B}, two of R^{C}, or two of R^{D} are joined, and at least one of R^{A}, R^{B}, R^{C}, or R^{D} comprises a bridged polycyclic ring.

In some embodiments, no two of R^{A}, two of R^{B}, two of R^{C}, or two of R^{D} are joined, and at least one of R^{A}, R^{B}, R^{C}, or R^{D} comprises an alkyl or cycloalkyl comprising five or more carbons.

In some embodiments, no two of R^{A}, two of R^{B}, two of R^{C}, or two of R^{D} are joined, and at least one of R^{A}, R^{B}, R^{C}, or R^{D} comprises an aromatic ring fused by a non-aromatic ring.

In some embodiments, two of R^{A}, two of R^{B}, two of R^{C}, or two of R^{D} are joined to form a benzene ring, and at least one of R^{A}, R^{B}, R^{C}, or R^{D} comprises a structure selected from the group consisting of deuterium, silyl, germyl, tetraphenylene, benzo[d]benzo[4,5]imidazo[1,2-a]imidazole (bimbim), a bridged polycyclic ring, an alkyl or cycloalkyl comprising five or more carbons, an aromatic ring fused by a non-aromatic ring, Formula III, and Formula IV.

In some embodiments, two of R^{A}, two of R^{B}, two of R^{C}, or two of R^{D} are joined to form a benzene ring, and at least one of R^{A}, R^{B}, R^{C}, or R^{D} comprises a deuterium.

In some embodiments, two of R^{A}, two of R^{B}, two of R^{C}, or two of R^{D} are joined to form a benzene ring, and at least one of R^{A}, R^{B}, R^{C}, or R^{D} comprises a silyl.

In some embodiments, two of R^{A}, two of R^{B}, two of R^{C}, or two of R^{D} are joined to form a benzene ring, and at least one of R^{A}, R^{B}, R^{C}, or R^{D} comprises a germyl.

In some embodiments, two of R^{A}, two of R^{B}, two of R^{C}, or two of R^{D} are joined to form a benzene ring, and at least one of R^{A}, R^{B}, R^{C}, or R^{D} comprises a tetraphenylene.

In some embodiments, two of R^{A}, two of R^{B}, two of R^{C}, or two of R^{D} are joined to form a benzene ring, and at least one of R^{A}, R^{B}, R^{C}, or R^{D} comprises a benzo[d]benzo[4,5]imidazo[1,2-a]imidazole (bimbim).

In some embodiments, two of R^{A}, two of R^{B}, two of R^{C}, or two of R^{D} are joined to form a benzene ring, and at least one of R^{A}, R^{B}, R^{C}, or R^{D} comprises a bicyclic alkyl.

In some embodiments, two of R^{A}, two of R^{B}, two of R^{C}, or two of R^{D} are joined to form a benzene ring, and at least one of R^{A}, R^{B}, R^{C}, or R^{D} comprises a bridged polycyclic ring.

In some embodiments, two of R^{A}, two of R^{B}, two of R^{C}, or two of R^{D} are joined to form a benzene ring, and at least one of R^{A}, R^{B}, R^{C}, or R^{D} comprises an alkyl or cycloalkyl comprising five or more carbons.

In some embodiments, two of R^{A}, two of R^{B}, two of R^{C}, or two of R^{D} are joined to form a benzene ring, and at least one of R^{A}, R^{B}, R^{C}, or R^{D} comprises an aromatic ring fused by a non-aromatic ring.

In some embodiments, two of R^{A}, two of R^{B}, two of R^{C}, or two of R^{D} are joined to form a benzene ring, and at least one of R^{A}, R^{B}, R^{C}, or R^{D} comprises a structure of Formula III.

In some embodiments, two of R^{A}, two of R^{B}, two of R^{C}, or two of R^{D} are joined to form a benzene ring, and at least one of R^{A}, R^{B}, R^{C}, or R^{D} comprises a structure of Formula III, wherein Y² is O.

In some embodiments, two of R^{A}, two of R^{B}, two of R^{C}, or two of R^{D} are joined to form a benzene ring, and at least one of R^{A}, R^{B}, R^{C}, or R^{D} comprises a structure of Formula III, wherein Y² is S.

In some embodiments, two of R^{A}, two of R^{B}, two of R^{C}, or two of R^{D} are joined to form a benzene ring, and at least one of R^{A}, R^{B}, R^{C}, or R^{D} comprises a structure of Formula III, wherein Y² is NR.

In some embodiments, two of R^{A}, two of R^{B}, two of R^{C}, or two of R^{D} are joined to form a benzene ring, and at least one of R^{A}, R^{B}, R^{C}, or R^{D} comprises a structure of Formula III, wherein at least one of X¹-X²⁴ is N.

In some embodiments, two of R^{A}, two of R^{B}, two of R^{C}, or two of R^{D} are joined to form a benzene ring, and at least one of R^{A}, R^{B}, R^{C}, or R^{D} comprises a structure of Formula III, wherein all of X¹-X²⁴ are C.

In some embodiments, two of R^{A}, two of R^{B}, two of R^{C}, or two of R^{D} are joined to form a benzene ring, and at least one of R^{A}, R^{B}, R^{C}, or R^{D} comprises a structure of Formula IV.

In some embodiments, two of R^{A}, two of R^{B}, two of R^{C}, or two of R^{D} are joined to form a benzene ring, and at least one of R^{A}, R^{B}, R^{C}, or R^{D} comprises a structure of Formula IV, wherein Y³ is O.

In some embodiments, two of R^{A}, two of R^{B}, two of R^{C}, or two of R^{D} are joined to form a benzene ring, and at least one of R^{A}, R^{B}, R^{C}, or R^{D} comprises a structure of Formula IV, wherein Y⁴ is O.

In some embodiments, two of R^{A}, two of R^{B}, two of R^{C}, or two of R^{D} are joined to form a benzene ring, and at least one of R^{A}, R^{B}, R^{C}, or R^{D} comprises a structure of Formula IV, wherein at least one of Y³ and Y⁴ is not O.

In some embodiments, two of R^{A}, two of R^{B}, two of R^{C}, or two of R^{D} are joined to form a benzene ring, and at least one of R^{A}, R^{B}, R^{C}, or R^{D} comprises a structure of Formula IV, wherein at least one of X²⁵-X³⁵ is N.

In some embodiments, two of R^{A}, two of R^{B}, two of R^{C}, or two of R^{D} are joined to form a benzene ring, and at least one of R^{A}, R^{B}, R^{C}, or R^{D} comprises a structure of Formula IV, wherein all of X²⁵-X³⁵ are C.

In some embodiments, the compound is selected from the group consisting of the following structures (LIST 1c) and at least one of R^{A}, R^{B}, R^{C}, or R^{D} comprises a structure selected from the group consisting of deuterium, silyl, germyl, tetraphenylene, benzo[d]benzo[4,5]imidazo[1,2-a]imidazole (bimbim), a bridged polycyclic ring, an alkyl or cycloalkyl comprising five or more carbons, an aromatic ring fused by a non-aromatic ring, Formula III, and Formula IV:

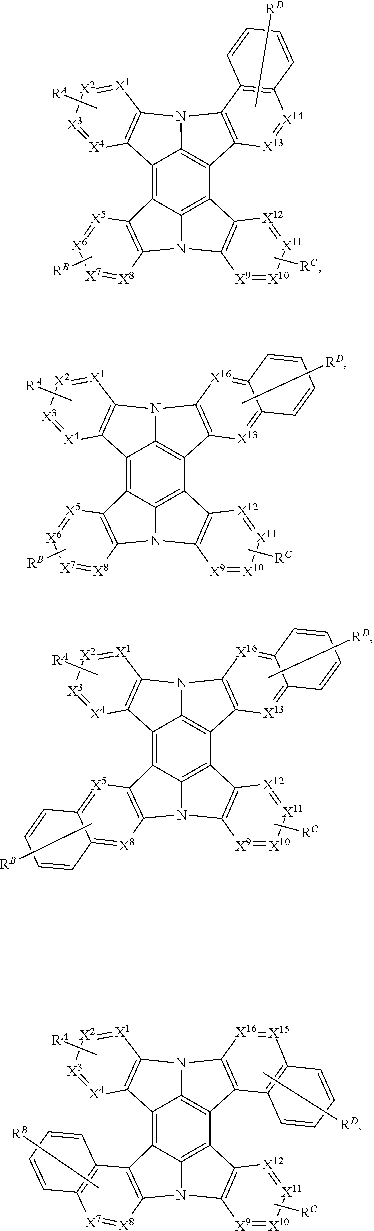

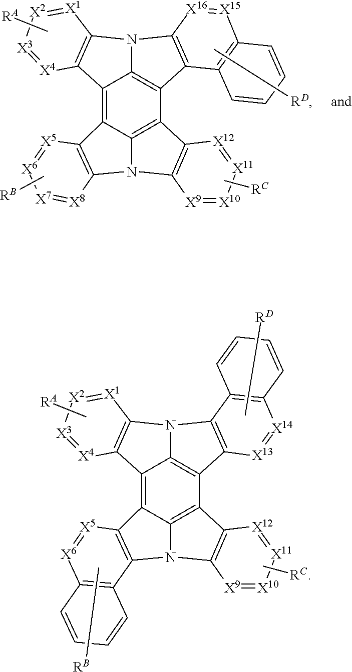

In some embodiments, X¹ to X¹⁶ are all C.

In some embodiments, two of R^{A}, two of R^{B}, two of R^{C}, or two of R^{D} are joined to form an 5 or 6-membered cycloalkyl ring, and at least one of R^{A}, R^{B}, R^{C}, or R^{D} comprises a structure selected from the group consisting of deuterium, alkyldiarylsilyl, dialkylarylsilyl, triarylsilane, substituted triphenylsilyl, germyl, tetraphenylene, benzo[d]benzo[4,5]imidazo[1,2-a]imidazole (bimbim), bridged polycyclic ring, an alkyl or cycloalkyl comprising five or more carbons, an aromatic ring fused by a non-aromatic ring, and Formula V.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises a deuterium.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises an alkyldiarylsilyl.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises a dialkylarylsilyl.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises a triarylsilane.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises a substituted triphenylsilyl.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises a germyl.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises a tetraphenylene.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises a benzo[d]benzo[4,5]imidazo[1,2-a]imidazole (bimbim).

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises an aromatic ring fused by a non-aromatic ring.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises a bicyclic alkyl.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises bridged polycyclic ring.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises an alkyl or cycloalkyl comprising five or more carbons.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises a structure of Formula V.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises a structure of Formula V, wherein Y⁵ is O.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises a structure of Formula V, wherein Y⁵ is S.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises a structure of Formula V, wherein at least one of X³⁶-X⁴³ is N.

In some embodiments, two of R^{A}, two of R^{B}, two of R^{C}, or two of R^{D} are joined to form a 5 or 6-membered alkyl ring, and at least one of R^{A}, R^{B}, R^{C}, or R^{D} comprises a structure of Formula V, wherein all of X³⁶-X⁴³ are C.

In some embodiments, the compound is selected from the group consisting of the following structures (LIST 1d), and at least one of R^{A}, R^{B}, R^{C}, or R^{D} comprises a structure selected from the group consisting of deuterium, alkyldiarylsilyl, dialkylarylsilyl, triarylsilane, substituted triphenylsilyl, germyl, tetraphenylene, benzo[d]benzo[4,5]imidazo[1,2-a]imidazole (bimbim), bridged polycyclic ring, an alkyl or cycloalkyl comprising five or more carbons, an aromatic ring fused by a non-aromatic ring, and Formula V:

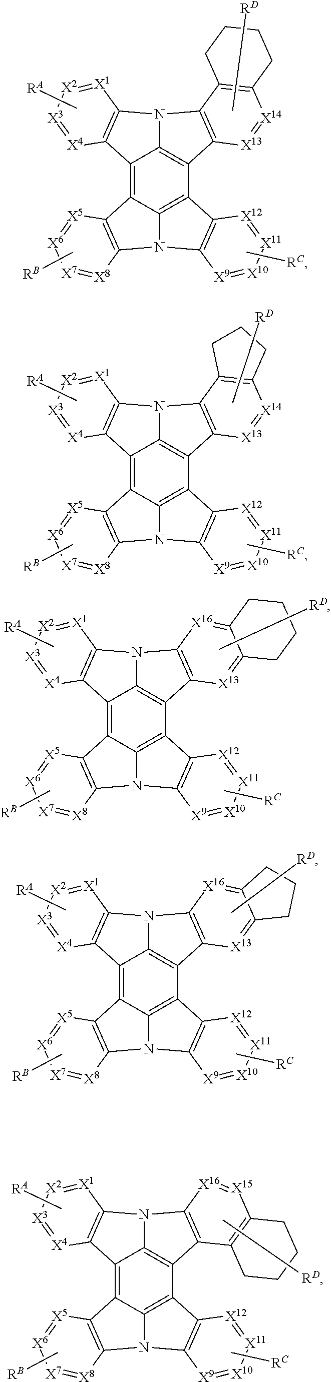

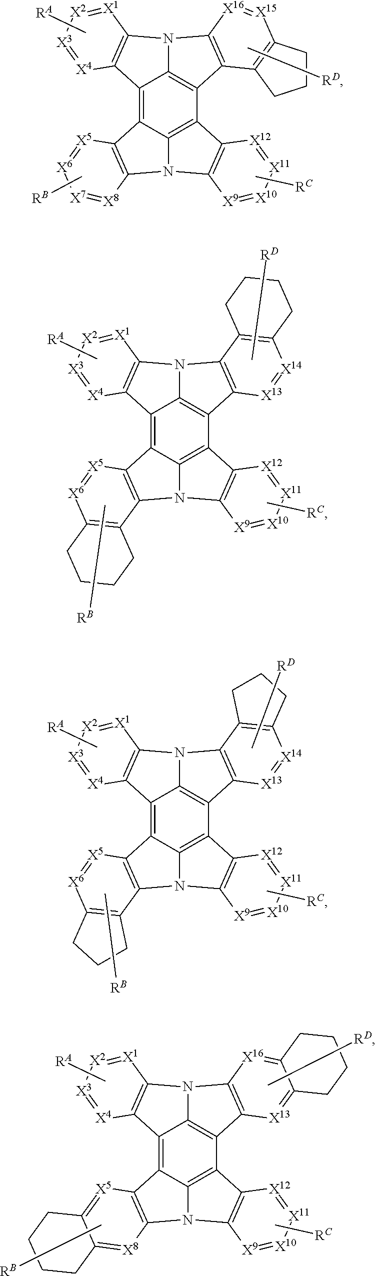

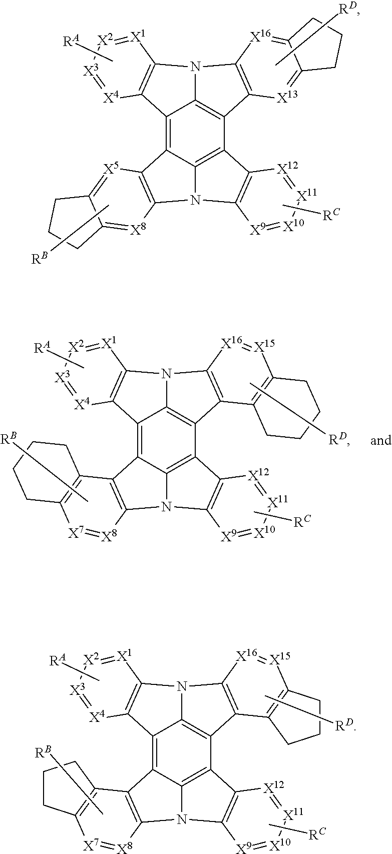

In some embodiments, X¹ to X¹⁶ are all C.

In some embodiments, the compound comprises an electron-withdrawing group selected from the group consisting of the following EWG1 LIST: F, CF₃, CN, COCH₃, CHO, COCF₃, COOMe, COOCF₃, NO₂, SF₃, SiF₃, PF₄, SFs, OCF₃, SCF₃, SeCF₃, SOCF₃, SeOCF₃, SO₂F, SO₂CF₃, SeO₂CF₃, OSeO₂CF₃, OCN, SCN, SeCN, NC, ⁺N(R^{k2})₃, (R^{k2})₂CCN, (R^{k2})₂CCF₃, CNC(CF₃)₂, BR^{k3}R^{k2}, substituted or unsubstituted dibenzoborole, 1-substituted carbazole, 1,9-substituted carbazole, substituted or unsubstituted carbazole, substituted or unsubstituted pyridine, substituted or unsubstituted pyrimidine, substituted or unsubstituted pyrazine, substituted or unsubstituted pyridoxine, substituted or unsubstituted triazine, substituted or unsubstituted oxazole, substituted or unsubstituted benzoxazole, substituted or unsubstituted thiazole, substituted or unsubstituted benzothiazole, substituted or unsubstituted imidazole, substituted or unsubstituted benzimidazole, ketone, carboxylic acid, ester, nitrile, isonitrile, sulfinyl, sulfonyl, partially and fully fluorinated alkyl, partially and fully fluorinated aryl, partially and fully fluorinated heteroaryl, cyano-containing alkyl, cyano-containing aryl, cyano-containing heteroaryl, isocyanate,

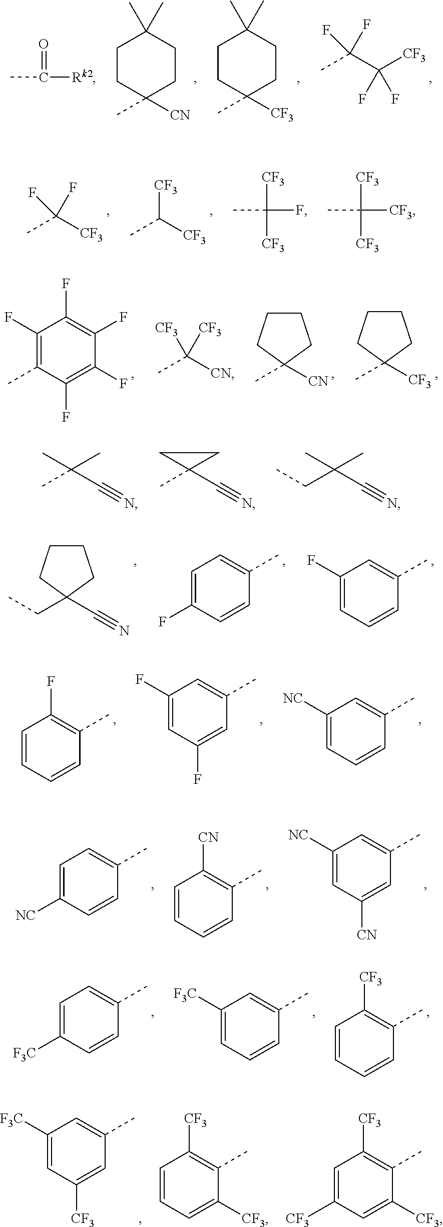

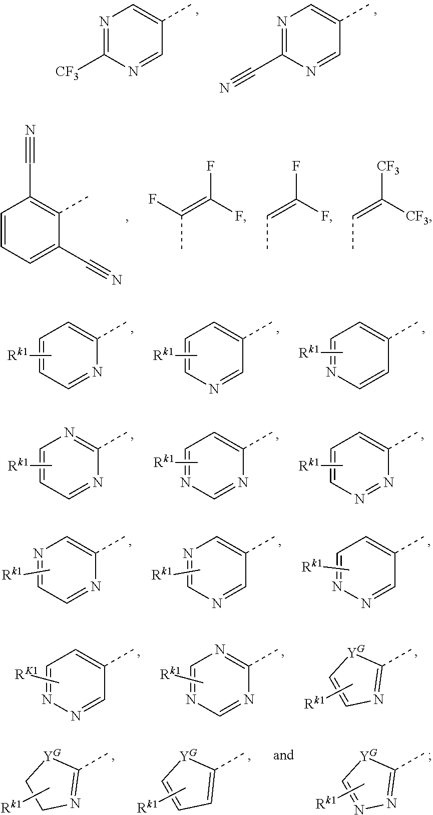

wherein each R^{k1} represents mono to the maximum allowable substitution, or no substitutions;
wherein Y^{G} is selected from the group consisting of BRₑ, NRₑ, PRₑ, O, S, Se, C═O, S═O, SO₂, CRₑR_{f}, SiRₑR_{f}, and GeRₑR_{f}; and
wherein each of R^{k1}, R^{k2}, R^{k3}, Rₑ, and R_{f} is independently a hydrogen or a substituent selected from the group consisting of the General Substituents defined herein.

In some embodiments, the compound comprises an electron-withdrawing group selected from the group consisting of the structures of the following EWG2 LIST:

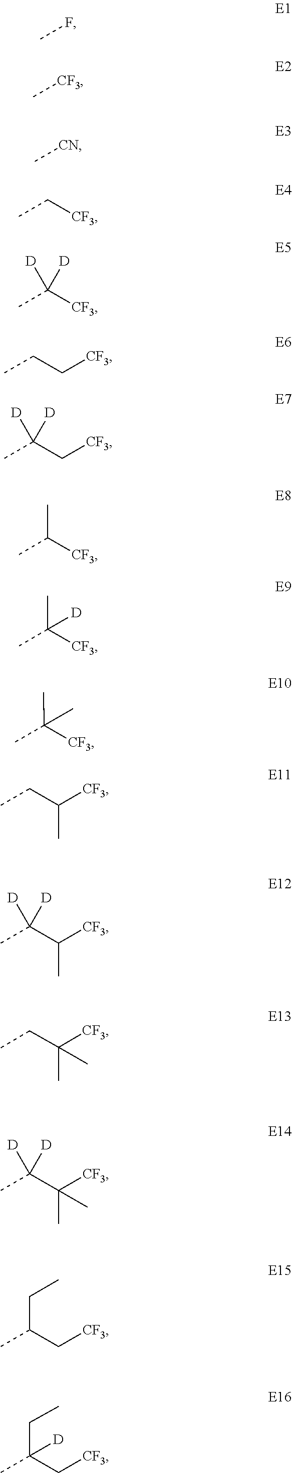

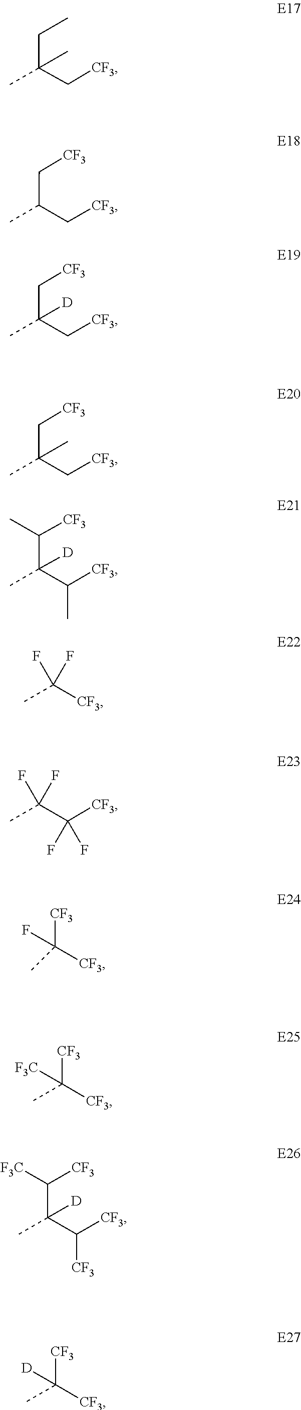

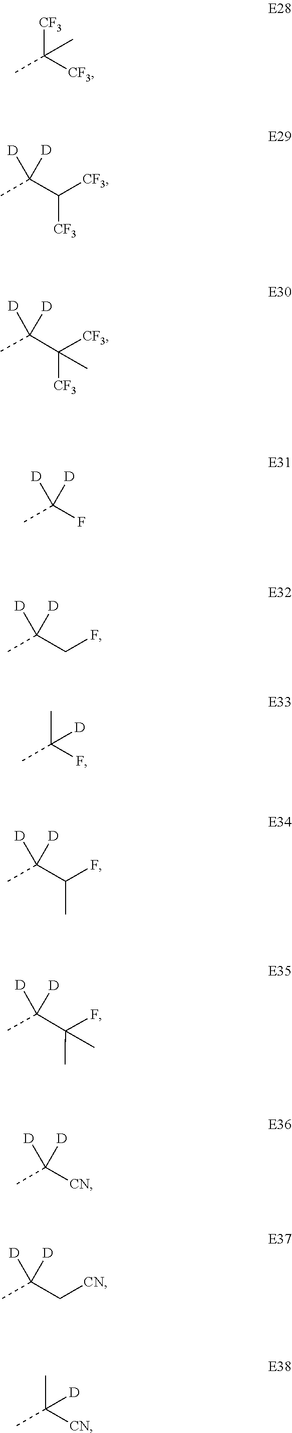

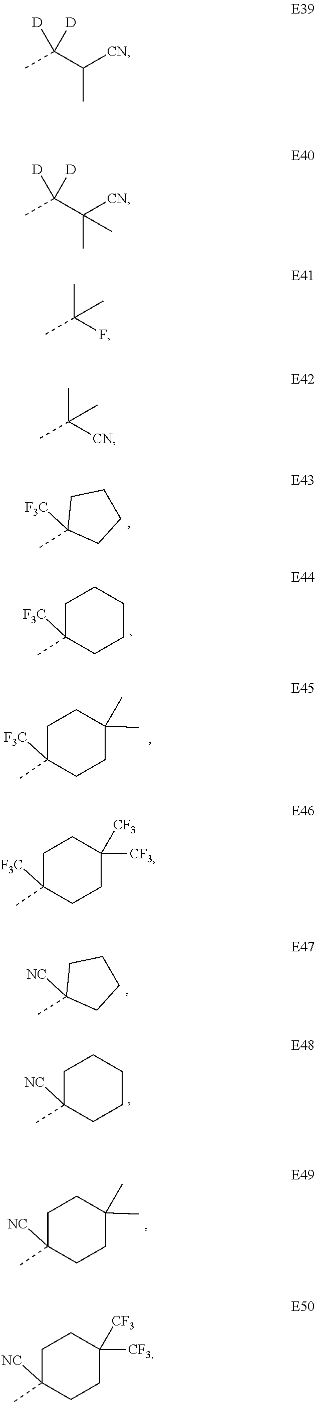

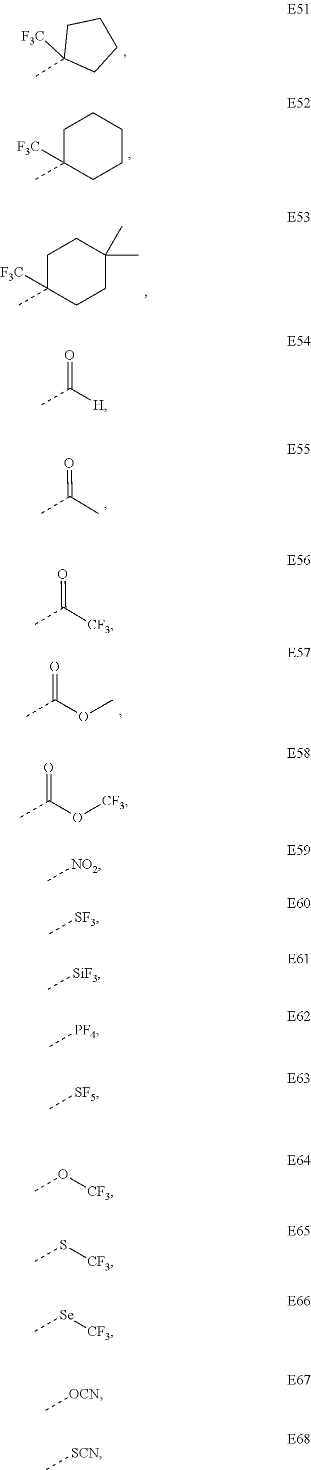

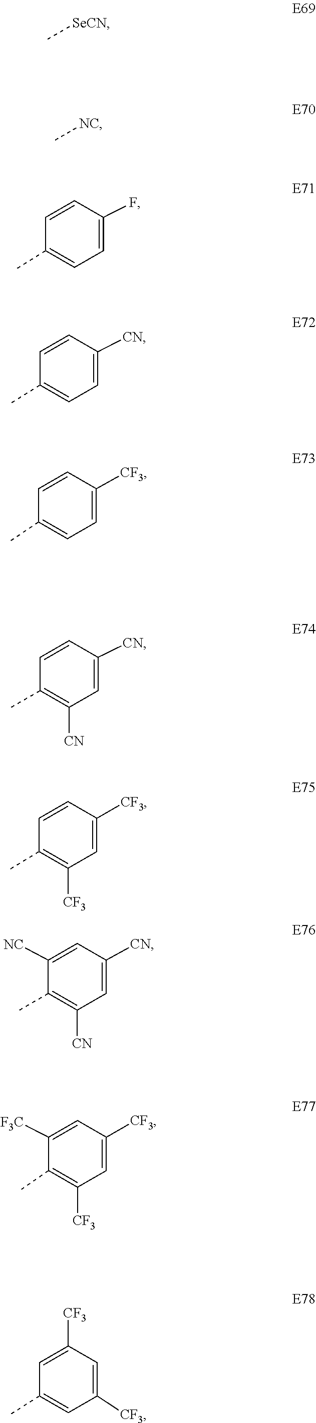

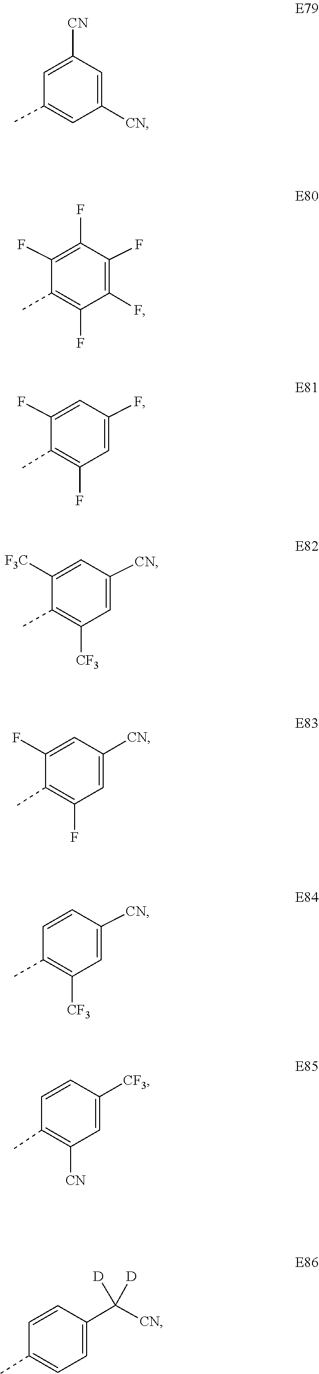

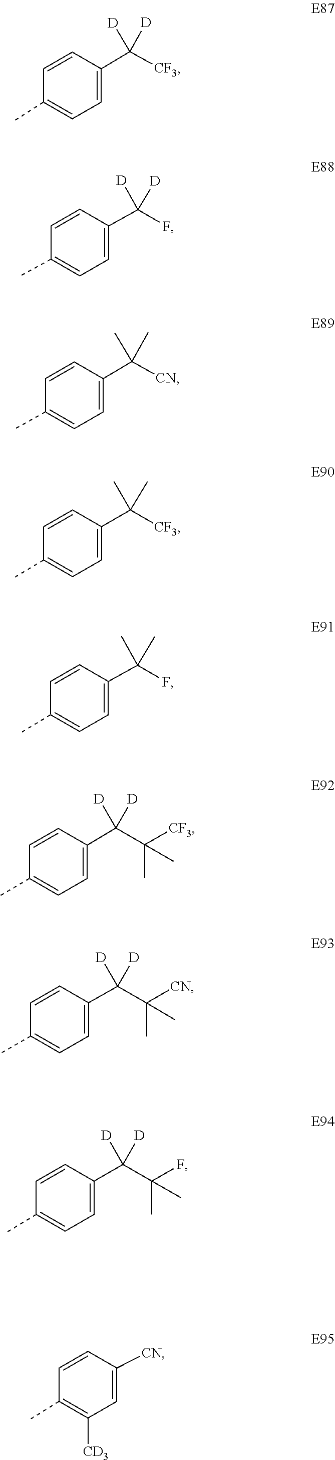

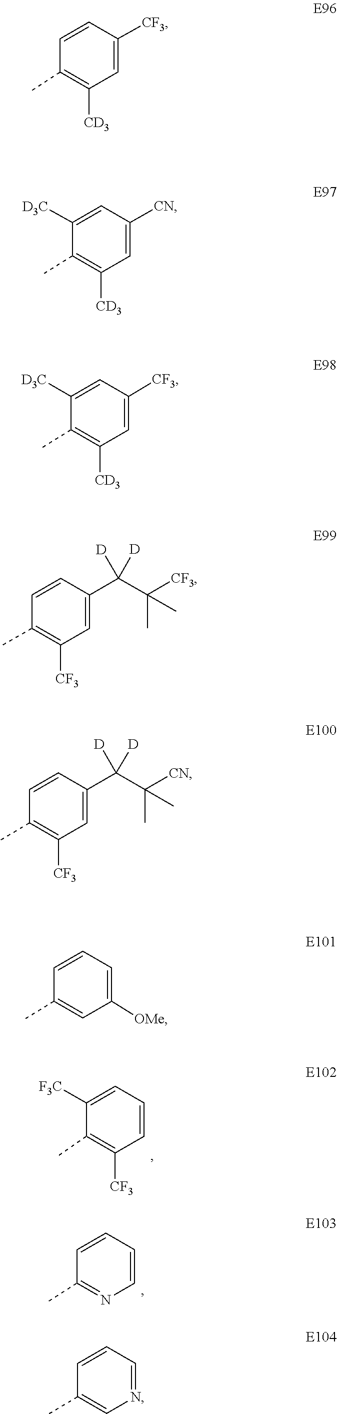

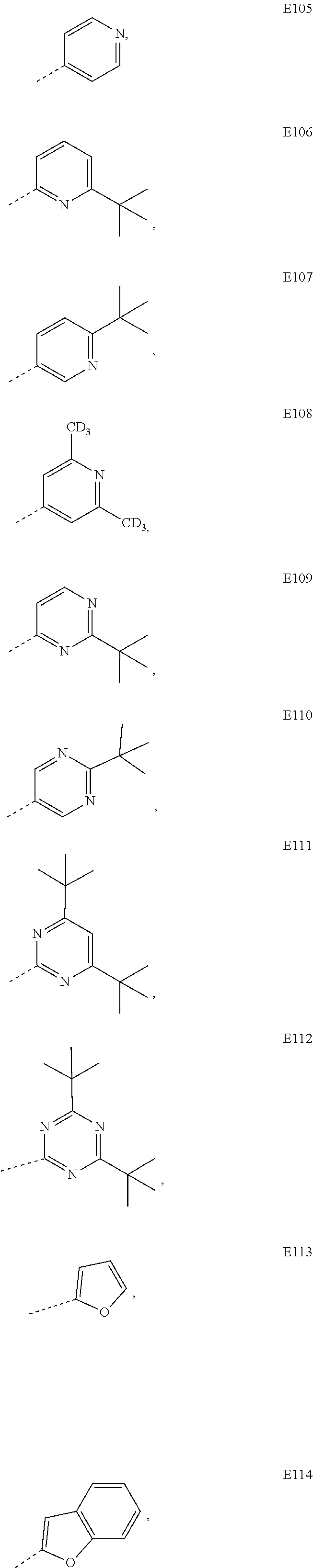

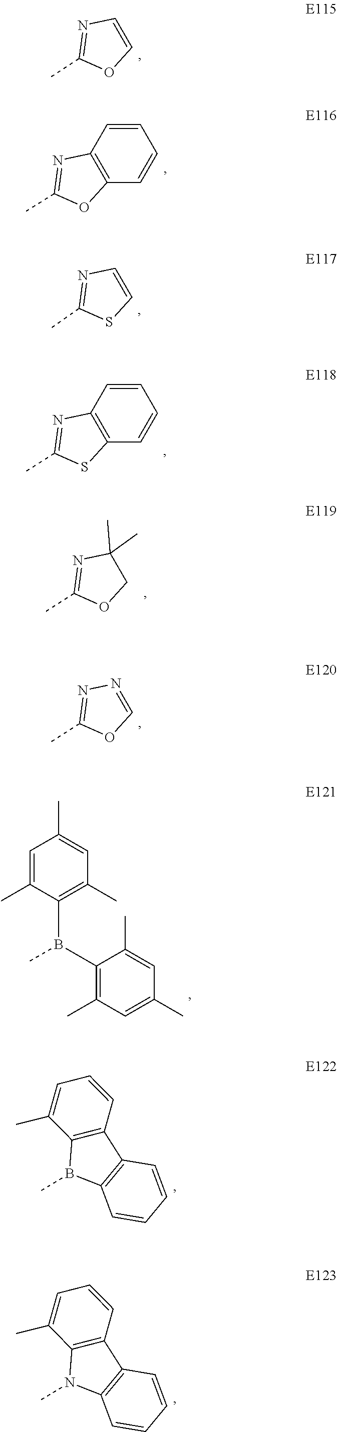

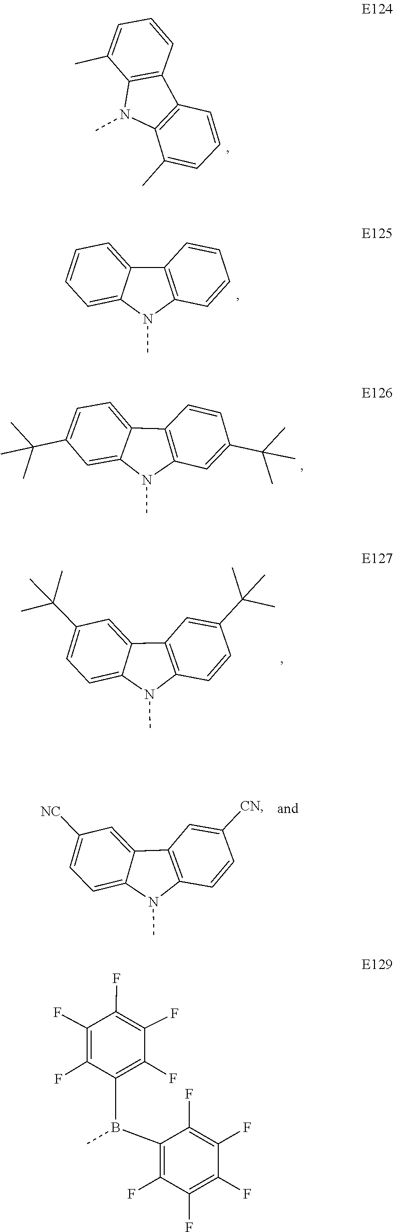

In some embodiments, the compound comprises an electron-withdrawing group selected from the group consisting of the structures of the following EWG3 LIST:

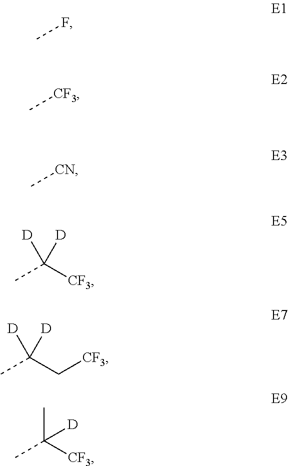

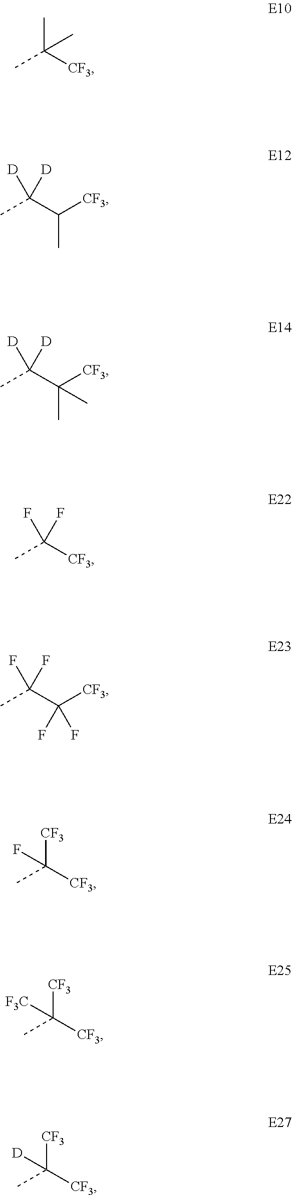

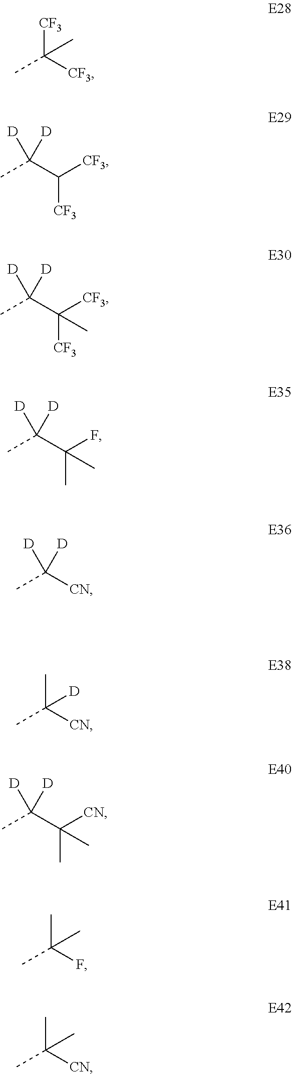

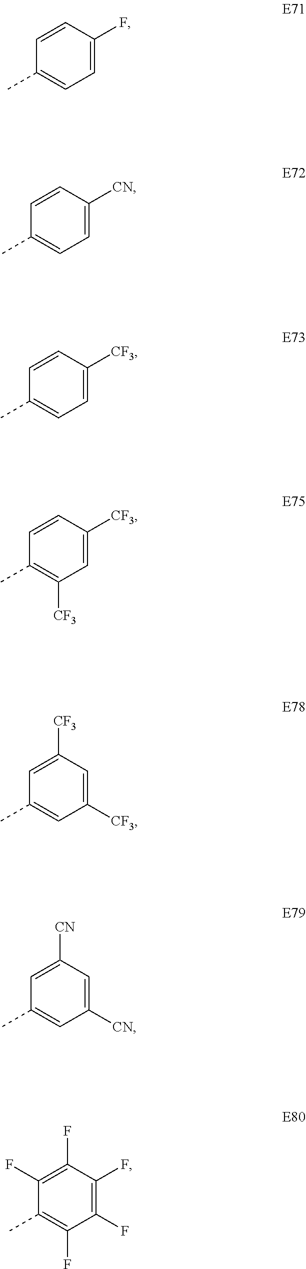

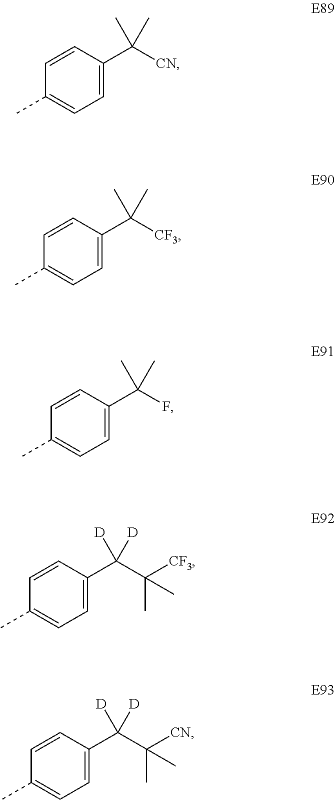

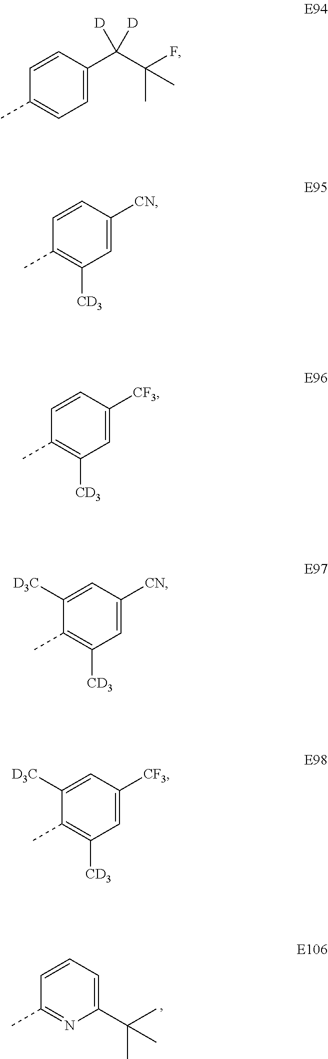

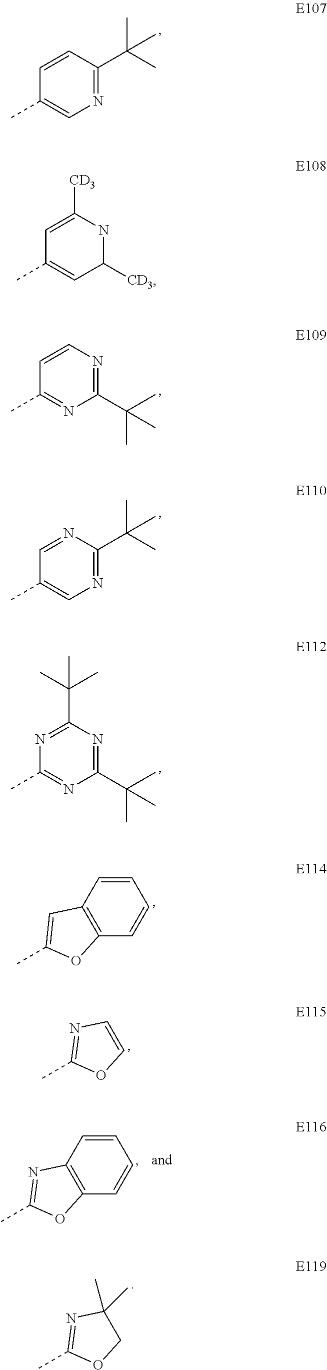

In some embodiments, the compound comprises an electron-withdrawing group selected from the group consisting of the structures of the following EWG4 LIST:

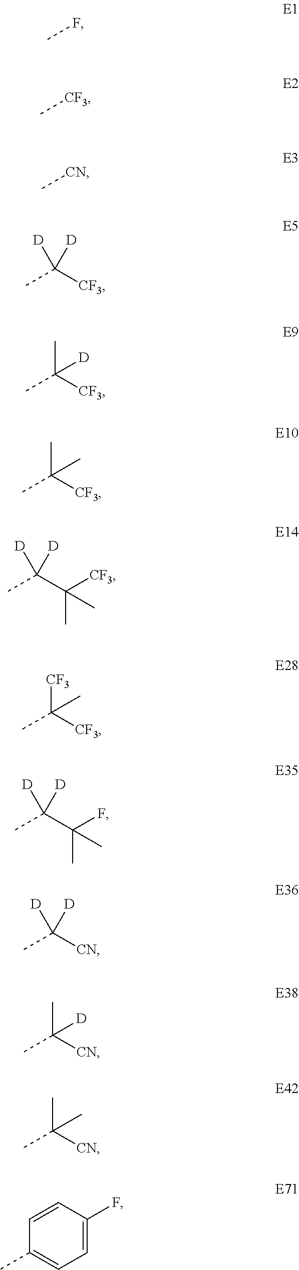

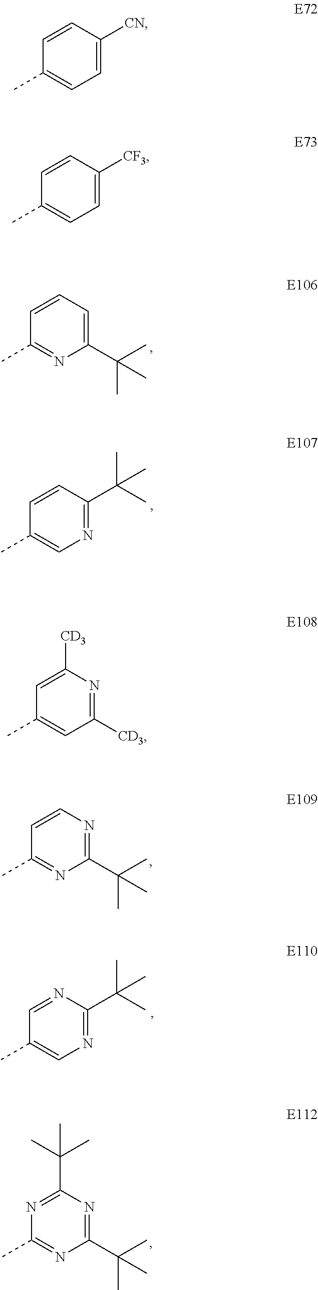

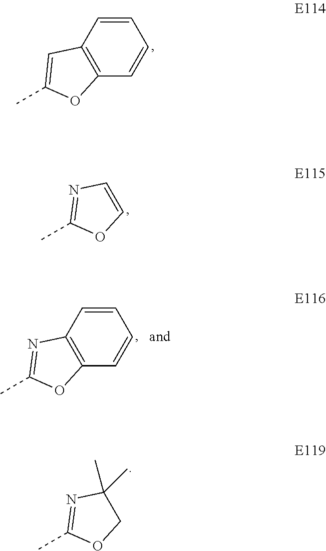

In some embodiments, the compound comprises a π-electron deficient electron-withdrawing group selected from the group consisting of the structures of the following Pi-EWG LIST: CN, COCH₃, CHO, COCF₃, COOMe, COOCF₃, NO₂, SF₃, SiF₃, PF₄, SFs, OCF₃, SCF₃, SeCF₃, SOCF₃, SeOCF₃, SO₂F, SO₂CF₃, SeO₂CF₃, OSeO₂CF₃, OCN, SCN, SeCN, NC, ⁺N(R^{k2})₃, BR^{k2}R^{k3}, substituted or unsubstituted dibenzoborole, 1-substituted carbazole, 1,9-substituted carbazole, substituted or unsubstituted carbazole, substituted or unsubstituted pyridine, substituted or unsubstituted pyrimidine, substituted or unsubstituted pyrazine, substituted or unsubstituted pyridazine, substituted or unsubstituted triazine, substituted or unsubstituted oxazole, substituted or unsubstituted benzoxazole, substituted or unsubstituted thiazole, substituted or unsubstituted benzothiazole, substituted or unsubstituted imidazole, substituted or unsubstituted benzimidazole, ketone, carboxylic acid, ester, nitrile, isonitrile, sulfinyl, sulfonyl, partially and fully fluorinated aryl, partially and fully fluorinated heteroaryl, cyano-containing aryl, cyano-containing heteroaryl, isocyanate,

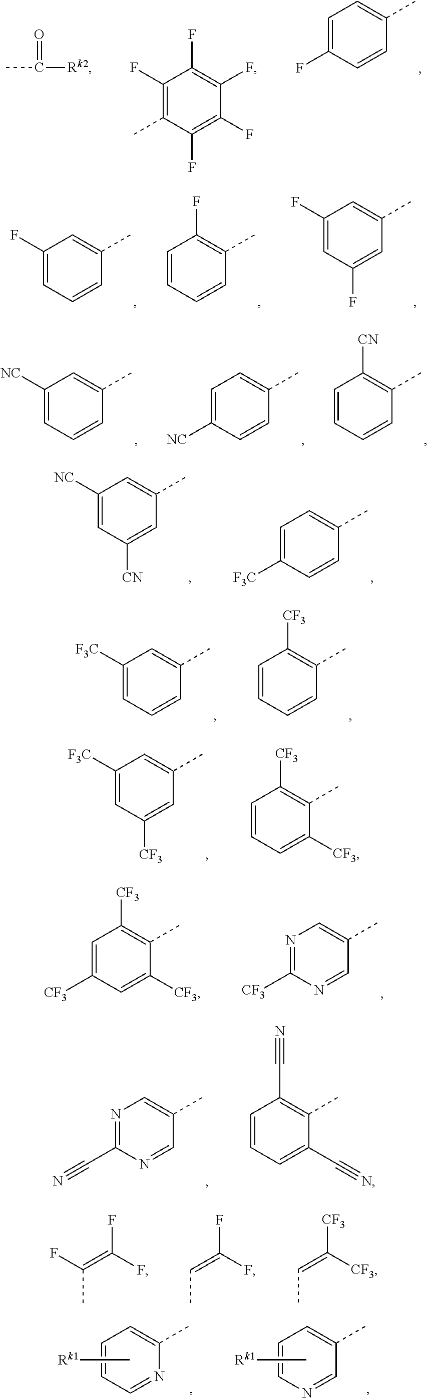

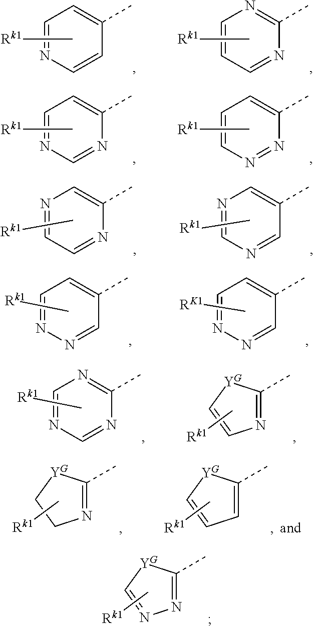

wherein the variables are the same as previously defined.

In some embodiments, at least one of R^{Y1}, R^{Y2}, R^{Y3}, R^{Y4}, R^{Y5}, R^{A}, R^{B}, R^{C}, R^{D}, and R^{E} is or comprises an electron-withdrawing group from the EWG1 LIST as defined herein. In some embodiments, at least one of R^{Y1}, R^{Y2}, R^{Y3}, R^{Y4}, R^{Y5}, R^{A}, R^{B}, R^{C}, R^{D}, and R^{E} is or comprises an electron-withdrawing group from the EWG2 LIST as defined herein. In some embodiments, at least one of R^{Y1}, R^{Y2}, R^{Y3}, R^{Y4}, R^{Y5}, R^{A}, R^{B}, R^{C}, R^{D}, and R^{E} is or comprises an electron-withdrawing group from the EWG3 LIST as defined herein. In some embodiments, at least one of R^{Y1}, R^{Y2}, R^{Y3}, R^{Y4}, R^{Y5}, R^{A}, R^{B}, R^{C}, R^{D}, and R^{E} is or comprises an electron-withdrawing group from the EWG4 LIST as defined herein. In some embodiments, at least one R^{Y1}, R^{Y2}, R^{Y3}, R^{Y4}, R^{Y5}, R^{A}, R^{B}, R^{C}, R^{D}, and R^{E} is or comprises an electron withdrawing group from the Pi-EWG LIST as defined herein.

In some embodiments, the compound has an E(S₁)-E(T₁) equal to or higher than 0.3 ev. E(S₁) refers to the first singlet energy, E(T₁) refers to the first triplet energy, E(S₁)-E(T₁) refers to the energy difference between S₁ and T₁.

In some embodiments, the compound has an E(S₁)-E(T₁) equal to or higher than 0.35 ev.

In some embodiments, the compound has an E(S₁)-E(T₁) equal to or higher than 0.4 ev.

In some embodiments, the compound is selected from the group consisting of Compound W1-(Ri)(Rj)(Rk)(Rl)(Ym), Compound W2-(Ri)(Rj)(Rk)(Rl)(Ym)(Yn), Compound W3-(Ri)(Rj)(Rl)(Ym)(Yn), Compound W4-(Ri)(Rj)(Rk)(Rl)(Ym), Compound W5-(Rj)(Rk)(Rl)(Rq), Compound W6-(Rj)(Rk)(Rl)(Rr), Compound W7-(Rj)(Rk)(Rl)(Rs), Compound W8-(Rj)(Rj)(Rk)(Rl), and Compound W9-(Rj)(Rj)(Rn′), wherein W1 is an integer from 1 to 14, W2 is an integer from 15 to 46, 49, 50, 53-60, and 93-144, W3 is 47, 48, 51, and 52, W4 is an integer from 61-92, W5 is an integer from 145-154, W6 is an integer from 155-222, W7 is an integer from 223-234, W8 is an integer from 235-341, and W9 is 342, i, j, k, n′, and l are an integer from 1 to 468; m and n are an integer from 1 to 51, q is an integer from 1 to 177, r is an integer from 1 to 232, s is an integer from 1 to 209, each of Ri, Rj, Rk, Rn′, and Rl is independently selected from the group consisting of R1 to R468; each of Rq is selected from the group consisting of R1 to R177; each of Rr is selected from the group consisting of R1 to R232; each of Rs is selected from the group consisting of R1 to R209; each of Ym and Yn is independently selected from the group consisting of Y1 to Y51; wherein each of the compounds are defined below in the following LIST 2:

| Compound | Structure of compound |
|---|---|
| Compound 1- (Ri)(Rj)(Rk)(Rl)(Ym), wherein Compound 1- (R1)(R1)(R1)(R1) (Y1) to Compound 1- (R468)(R468)(R468) (R468)(Y51), have the structure | 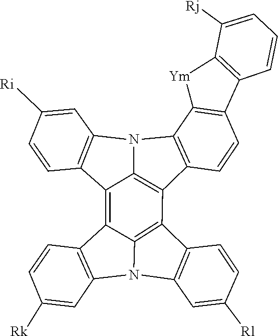 |
| Compound 2- (Ri)(Rj)(Rk)(Rl)(Ym), wherein Compound 2- (R1)(R1)(R1)(R1) (Y1) to Compound 2- (R468)(R468)(R468) (R468)(Y51), have the structure | 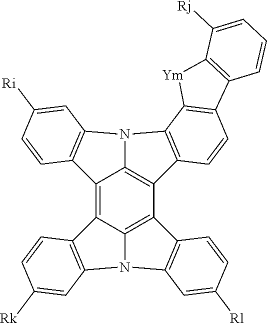 |
| Compound 3- (Ri)(Rj)(Rk)(Rl)(Ym), wherein Compound 3- (R1)(R1)(R1)(R1) (Y1) to Compound 3- (R468)(R468)(R468) (R468)(Y51), have the structure | 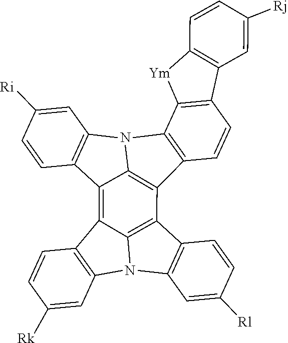 |
| Compound 4- (Ri)(Rj)(Rk)(Rl)(Ym), wherein Compound 4- (R1)(R1)(R1)(R1) (Y1) to Compound 4- (R468)(R468)(R468) (R468)(Y51), have the structure | 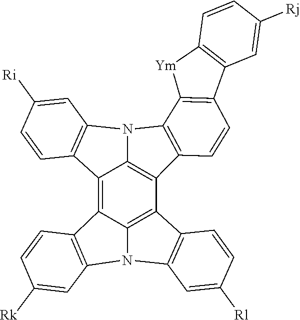 |
| Compound 5- (Ri)(Rj)(Rk)(Rl)(Ym), wherein Compound 5- (R1)(R1)(R1)(R1) (Y1) to Compound 5- (R468)(R468)(R468) (R468)(Y51), have the structure | 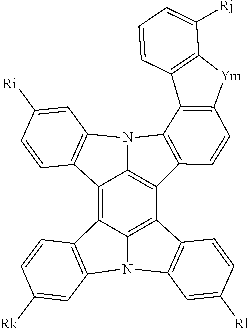 |
| Compound 6- (Ri)(Rj)(Rk)(Rl)(Ym), wherein Compound 6- (R1)(R1)(R1)(R1) (Y1) to Compound 6- (R468)(R468)(R468) (R468)(Y51), have the structure | 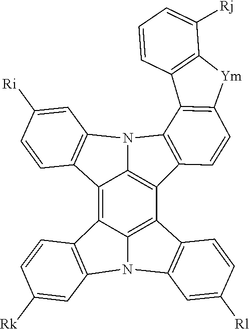 |
| Compound 7- (Ri)(Rj)(Rk)(Rl)(Ym), wherein Compound 7- (R1)(R1)(R1)(R1) (Y1) to Compound 7- (R468)(R468)(R468) (R468)(Y51), have the structure | 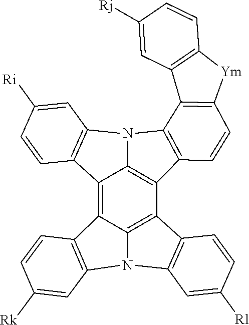 |
| Compound 8- (Ri)(Rj)(Rk)(Rl)(Ym), wherein Compound 8- (R1)(R1)(R1)(R1) (Y1) to Compound 8- (R468)(R468)(R468) (R468)(Y51), have the structure | 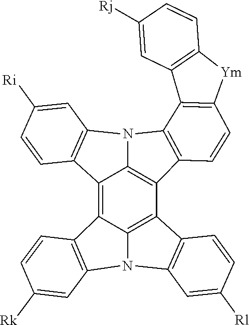 |
| Compound 9- (Ri)(Rj)(Rk)(Rl)(Ym), wherein Compound 9- (R1)(R1)(R1)(R1) (Y1) to Compound 9- (R468)(R468)(R468) (R468)(Y51), have the structure | 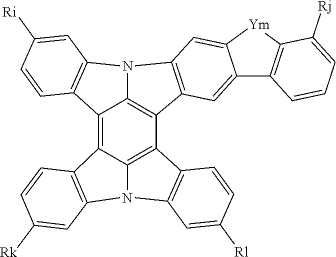 |
| Compound 10- (Ri)(Rj)(Rk)(Rl)(Ym), wherein Compound 10- (R1)(R1)(R1)(R1) (Y1) to Compound 10- (R468)(R468)(R468) (R468)(Y51), have the structure | 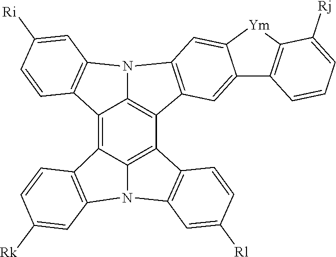 |
| Compound 11- (Ri)(Rj)(Rk)(Rl)(Ym), wherein Compound 11- (R1)(R1)(R1)(R1) (Y1) to Compound 11- (R468)(R468)(R468) (R468)(Y51), have the structure | 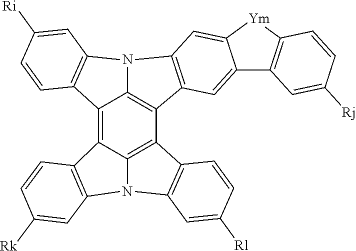 |
| Compound 12- (Ri)(Rj)(Rk)(Rl)(Ym), wherein Compound 12- (R1)(R1)(R1)(R1) (Y1) to Compound 12- (R468)(R468)(R468) (R468)(Y51), have the structure | 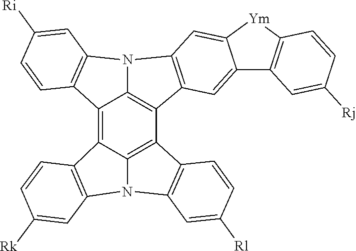 |
| Compound 13- (Ri)(Rj)(Rk)(Rl)(Ym), wherein Compound 13- (R1)(R1)(R1)(R1) (Y1) to Compound 13- (R468)(R468)(R468) (R468)(Y51), have the structure | 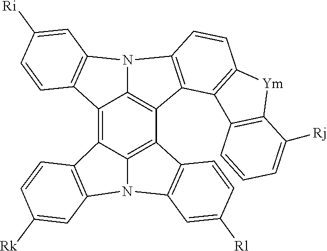 |
| Compound 14- (Ri)(Rj)(Rk)(Rl)(Ym), wherein Compound 14- (R1)(R1)(R1)(R1) (Y1) to Compound 14- (R468)(R468)(R468) (R468)(Y51), have the structure | 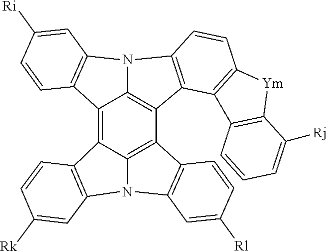 |
| Compound 15- (Ri)(Rj)(Rk)(Rl) (Ym)(Yn), wherein Compound 15- (R1)(R1)(R1)(R1)(Y1) (Y1) to Compound 15- (R468)(R468)(R468) (R468)(Y51)(Y51), have the structure | 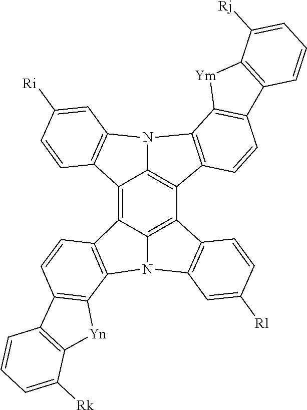 |
| Compound 16- (Ri)(Rj)(Rk)(Rl) (Ym)(Yn), wherein Compound 16- (R1)(R1)(R1)(R1)(Y1) (Y1) to Compound 16- (R468)(R468)(R468) (R468)(Y51)(Y51), have the structure | 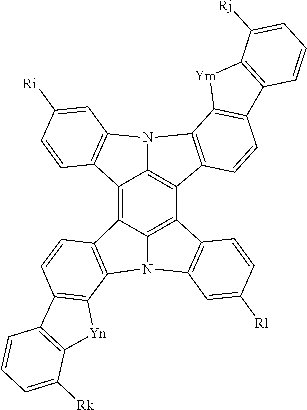 |
| Compound 17- (Ri)(Rj)(Rk)(Rl) (Ym)(Yn), wherein Compound 17- (R1)(R1)(R1)(R1)(Y1) (Y1) to Compound 17- (R468)(R468)(R468) (R468)(Y51)(Y51), have the structure | 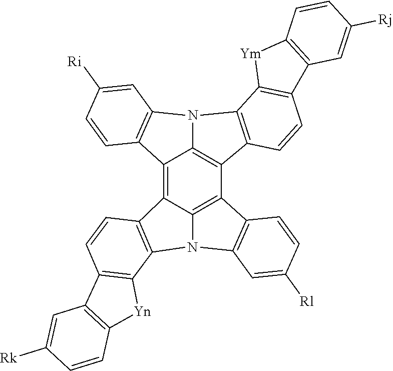 |
| Compound 18- (Ri)(Rj)(Rk)(Rl) (Ym)(Yn), wherein Compound 18- (R1)(R1)(R1)(R1)(Y1) (Y1) to Compound 18- (R468)(R468)(R468) (R468)(Y51)(Y51), have the structure | 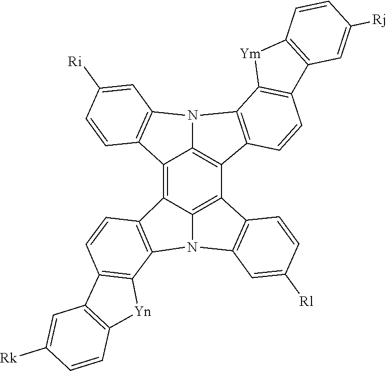 |
| Compound 19- (Ri)(Rj)(Rk)(Rl) (Ym)(Yn), wherein Compound 19- (R1)(R1)(R1)(R1)(Y1) (Y1) to Compound 19- (R468)(R468)(R468) (R468)(Y51)(Y51), have the structure | 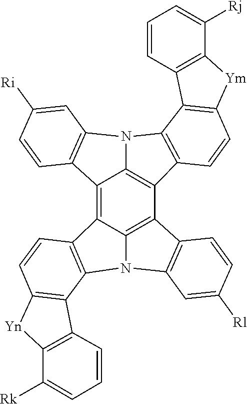 |
| Compound 20- (Ri)(Rj)(Rk)(Rl) (Ym)(Yn), wherein Compound 20- (R1)(R1)(R1)(R1)(Y1) (Y1) to Compound 20- (R468)(R468)(R468) (R468)(Y51)(Y51), have the structure | 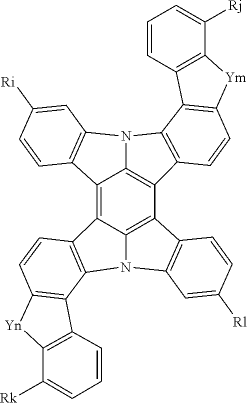 |
| Compound 21- (Ri)(Rj)(Rk)(Rl) (Ym)(Yn), wherein Compound 21- (R1)(R1)(R1)(R1)(Y1) (Y1) to Compound 21- (R468)(R468)(R468) (R468)(Y51)(Y51), have the structure | 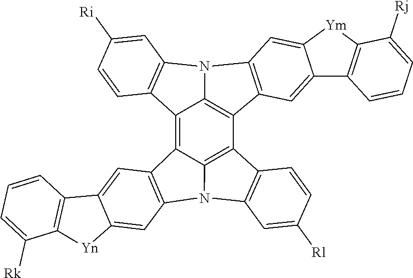 |
| Compound 22- (Ri)(Rj)(Rk)(Rl) (Ym)(Yn), wherein Compound 22- (R1)(R1)(R1)(R1)(Y1) (Y1) to Compound 22- (R468)(R468)(R468) (R468)(Y51)(Y51), have the structure | 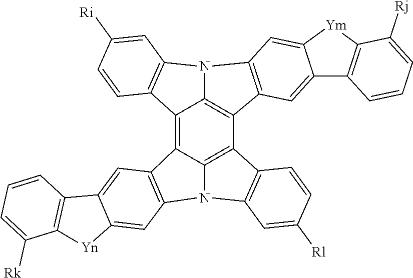 |
| Compound 23- (Ri)(Rj)(Rk)(Rl) (Ym)(Yn), wherein Compound 23- (R1)(R1)(R1)(R1)(Y1) (Y1) to Compound 23- (R468)(R468)(R468) (R468)(Y51)(Y51), have the structure | 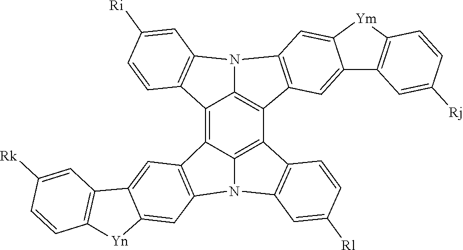 |
| Compound 24- (Ri)(Rj)(Rk)(Rl) (Ym)(Yn), wherein Compound 24- (R1)(R1)(R1)(R1)(Y1) (Y1) to Compound 24- (R468)(R468)(R468) (R468)(Y51)(Y51), have the structure | 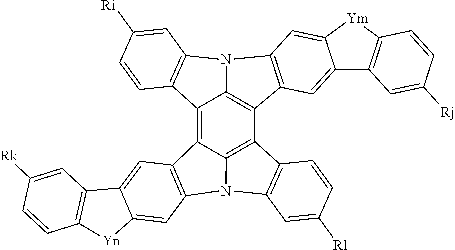 |
| Compound 25- (Ri)(Rj)(Rk)(Rl) (Ym)(Yn), wherein Compound 25- (R1)(R1)(R1)(R1)(Y1) (Y1) to Compound 25- (R468)(R468)(R468) (R468)(Y51)(Y51), have the structure | 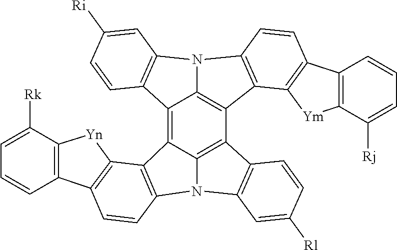 |
| Compound 26- (Ri)(Rj)(Rk)(Rl) (Ym)(Yn), wherein Compound 26- (R1)(R1)(R1)(R1)(Y1) (Y1) to Compound 26- (R468)(R468)(R468) (R468)(Y51)(Y51), have the structure | 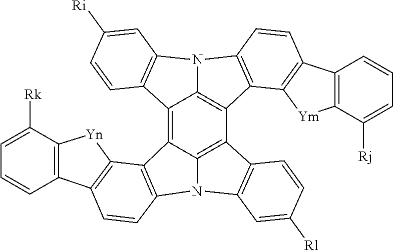 |
| Compound 27- (Ri)(Rj)(Rk)(Rl) (Ym)(Yn), wherein Compound 27- (R1)(R1)(R1)(R1)(Y1) (Y1) to Compound 27- (R468)(R468)(R468) (R468)(Y51)(Y51), have the structure | 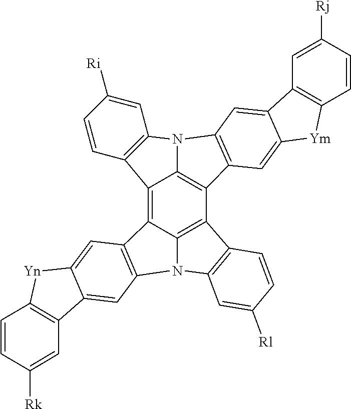 |
| Compound 28- (Ri)(Rj)(Rk)(Rl) (Ym)(Yn), wherein Compound 28- (R1)(R1)(R1)(R1)(Y1) (Y1) to Compound 28- (R468)(R468)(R468) (R468)(Y51)(Y51), have the structure | 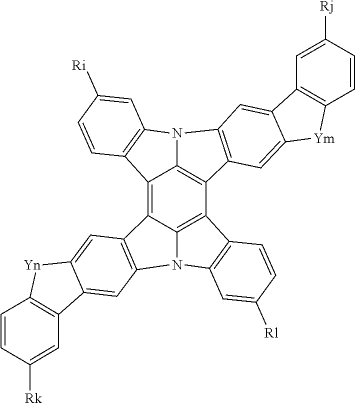 |
| Compound 29- (Ri)(Rj)(Rk)(Rl) (Ym)(Yn), wherein Compound 29- (R1)(R1)(R1)(R1)(Y1) (Y1) to Compound 29- (R468)(R468)(R468) (R468)(Y51)(Y51), have the structure | 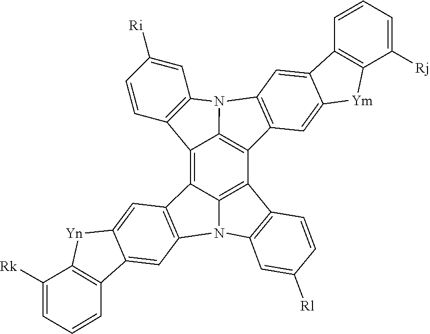 |
| Compound 30- (Ri)(Rj)(Rk)(Rl) (Ym)(Yn), wherein Compound 30- (R1)(R1)(R1)(R1)(Y1) (Y1) to Compound 30- (R468)(R468)(R468) (R468)(Y51)(Y51), have the structure | 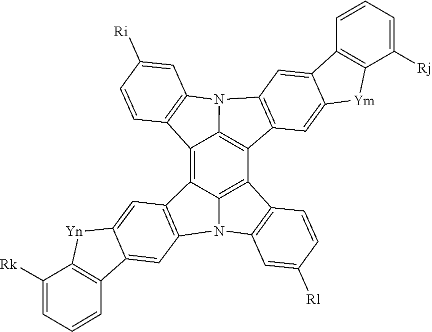 |
| Compound 31- (Ri)(Rj)(Rk)(Rl) (Ym)(Yn), wherein Compound 31- (R1)(R1)(R1)(R1)(Y1) (Y1) to Compound 31- (R468)(R468)(R468) (R468)(Y51)(Y51), have the structure | 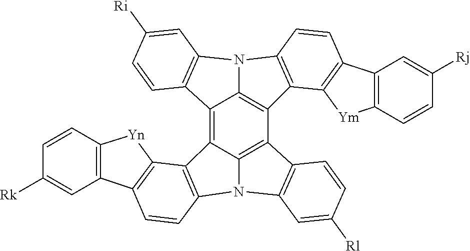 |
| Compound 32- (Ri)(Rj)(Rk)(Rl) (Ym)(Yn), wherein Compound 32- (R1)(R1)(R1)(R1)(Y1) (Y1) to Compound 32- (R468)(R468)(R468) (R468)(Y51)(Y51), have the structure | 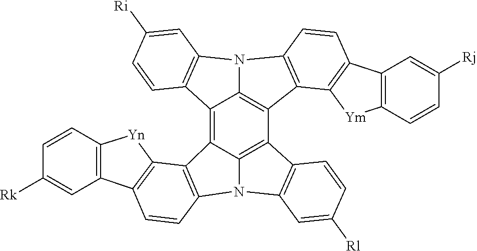 |
| Compound 33- (Ri)(Rj)(Rk)(Rl) (Ym)(Yn), wherein Compound 33- (R1)(R1)(R1)(R1)(Y1) (Y1) to Compound 33- (R468)(R468)(R468) (R468)(Y51)(Y51), have the structure | 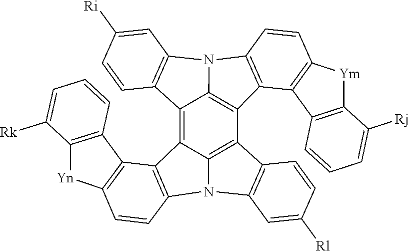 |
| Compound 34- (Ri)(Rj)(Rk)(Rl) (Ym)(Yn), wherein Compound 34- (R1)(R1)(R1)(R1)(Y1) (Y1) to Compound 34- (R468)(R468)(R468) (R468)(Y51)(Y51), have the structure | 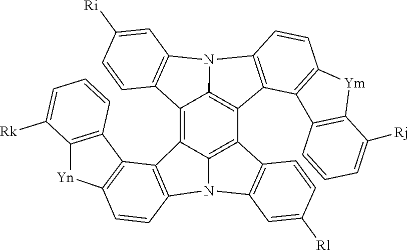 |
| Compound 35- (Ri)(Rj)(Rk)(Rl) (Ym)(Yn), wherein Compound 35- (R1)(R1)(R1)(R1)(Y1) (Y1) to Compound 35- (R468)(R468)(R468) (R468)(Y51)(Y51), have the structure | 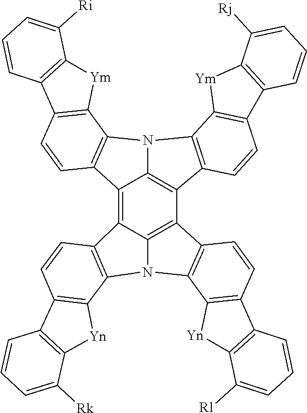 |
| Compound 36- (Ri)(Rj)(Rk)(Rl) (Ym)(Yn), wherein Compound 36- (R1)(R1)(R1)(R1)(Y1) (Y1) to Compound 36- (R468)(R468)(R468) (R468)(Y51)(Y51), have the structure | 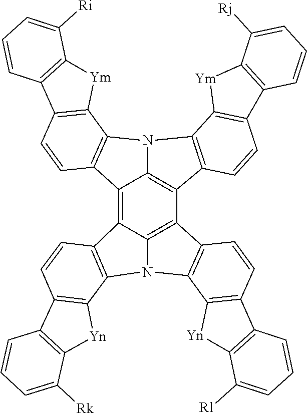 |
| Compound 37- (Ri)(Rj)(Rk)(Rl) (Ym)(Yn), wherein Compound 37- (R1)(R1)(R1)(R1)(Y1) (Y1) to Compound 37- (R468)(R468)(R468) (R468)(Y51)(Y51), have the structure | 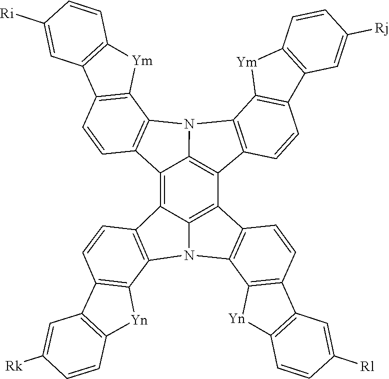 |
| Compound 38- (Ri)(Rj)(Rk)(Rl) (Ym)(Yn), wherein Compound 38- (R1)(R1)(R1)(R1)(Y1) (Y1) to Compound 38- (R468)(R468)(R468) (R468)(Y51)(Y51), have the structure | 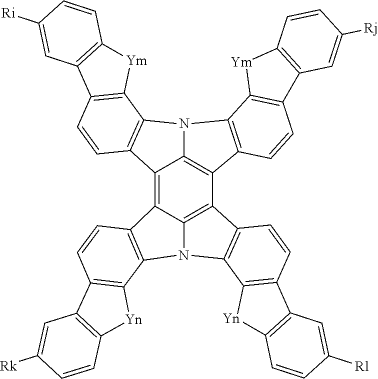 |
| Compound 39- (Ri)(Rj)(Rk)(Rl) (Ym)(Yn), wherein Compound 39- (R1)(R1)(R1)(R1)(Y1) (Y1) to Compound 39- (R468)(R468)(R468) (R468)(Y51)(Y51), have the structure | 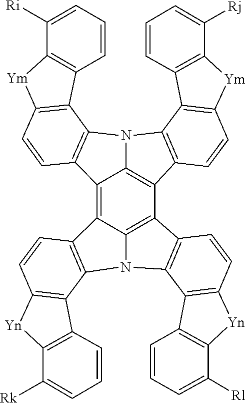 |
| Compound 40- (Ri)(Rj)(Rk)(Rl) (Ym)(Yn), wherein Compound 40- (R1)(R1)(R1)(R1)(Y1) (Y1) to Compound 40- (R468)(R468)(R468) (R468)(Y51)(Y51), have the structure | 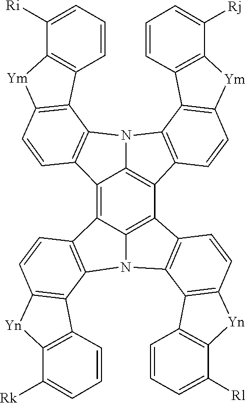 |
| Compound 41- (Ri)(Rj)(Rk)(Rl) (Ym)(Yn), wherein Compound 41- (R1)(R1)(R1)(R1)(Y1) (Y1) to Compound 41- (R468)(R468)(R468) (R468)(Y51)(Y51), have the structure | 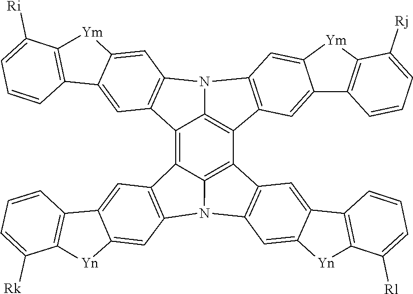 |
| Compound 42- (Ri)(Rj)(Rk)(Rl) (Ym)(Yn), wherein Compound 42- (R1)(R1)(R1)(R1)(Y1) (Y1) to Compound 42- (R468)(R468)(R468) (R468)(Y51)(Y51), have the structure | 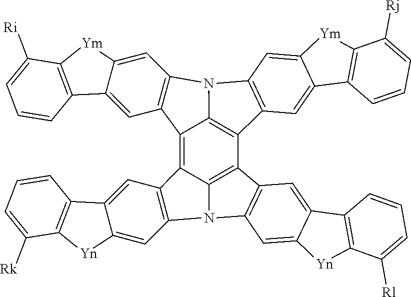 |
| Compound 43- (Ri)(Rj)(Rk)(Rl) (Ym)(Yn), wherein Compound 43- (R1)(R1)(R1)(R1)(Y1) (Y1) to Compound 43- (R468)(R468)(R468) (R468)(Y51)(Y51), have the structure | 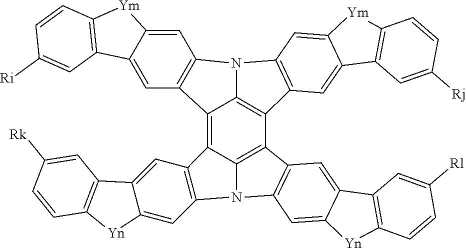 |
| Compound 44- (Ri)(Rj)(Rk)(Rl) (Ym)(Yn), wherein Compound 44- (R1)(R1)(R1)(R1)(Y1) (Y1) to Compound 44- (R468)(R468)(R468) (R468)(Y51)(Y51), have the structure | 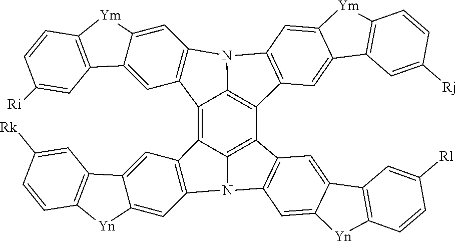 |
| Compound 45- (Ri)(Rj)(Rk)(Rl) (Ym)(Yn), wherein Compound 45- (R1)(R1)(R1)(R1)(Y1) (Y1) to Compound 45- (R468)(R468)(R468) (R468)(Y51)(Y51), have the structure | 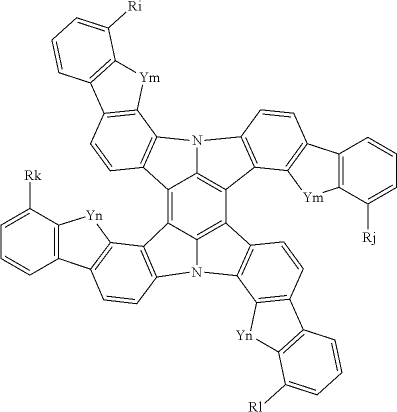 |
| Compound 46- (Ri)(Rj)(Rk)(Rl) (Ym)(Yn), wherein Compound 46- (R1)(R1)(R1)(R1)(Y1) (Y1) to Compound 46- (R468)(R468)(R468) (R468)(Y51)(Y51), have the structure | 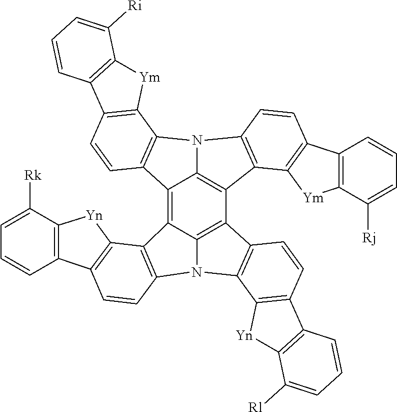 |
| Compound 47- (Ri)(Rj)(Rk)(Rl) (Ym)(Yn), wherein Compound 47- (R1)(R1)(R1)(R1)(Y1) (Y1) to Compound 47- (R468)(R468)(R468) (R468)(Y51)(Y51), have the structure | 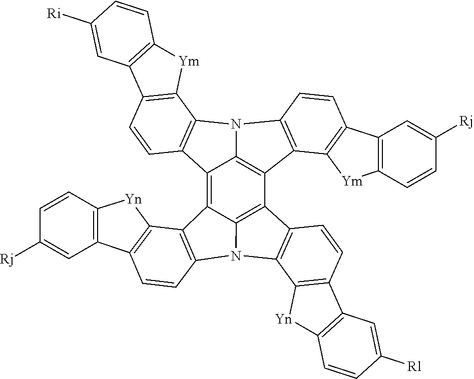 |
| Compound 48- (Ri)(Rj)(Rk)(Rl) (Ym)(Yn), wherein Compound 48- (R1)(R1)(R1)(R1)(Y1) (Y1) to Compound 48- (R468)(R468)(R468) (R468)(Y51)(Y51), have the structure | 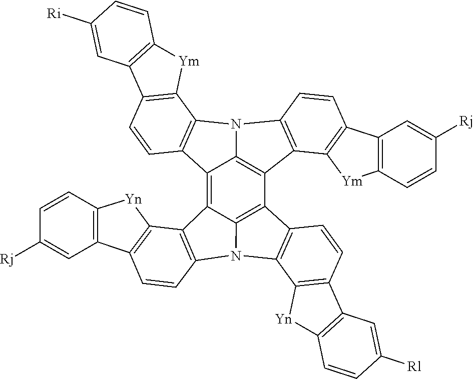 |
| Compound 49- (Ri)(Rj)(Rk)(Rl) (Ym)(Yn), wherein Compound 49- (R1)(R1)(R1)(R1)(Y1) (Y1) to Compound 49- (R468)(R468)(R468) (R468)(Y51)(Y51), have the structure | 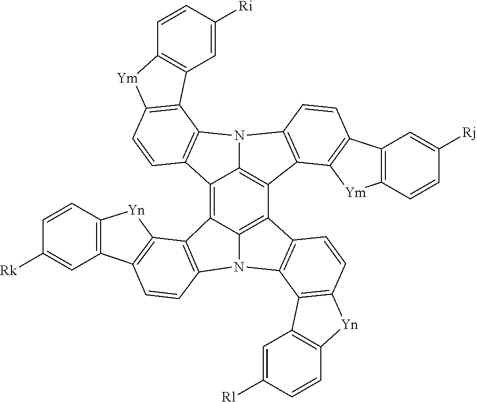 |
| Compound 50- (Ri)(Rj)(Rk)(Rl) (Ym)(Yn), wherein Compound 50- (R1)(R1)(R1)(R1)(Y1) (Y1) to Compound 50- (R468)(R468)(R468) (R468)(Y51)(Y51), have the structure | 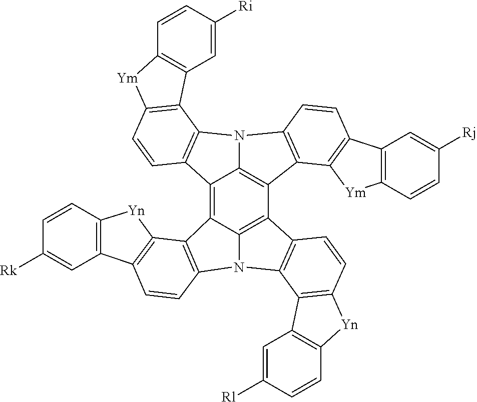 |
| Compound 51- (Ri)(Rj)(Rk)(Rl) (Ym)(Yn), wherein Compound 51- (R1)(R1)(R1)(R1)(Y1) (Y1) to Compound 51- (R468)(R468)(R468) (R468)(Y51)(Y51), have the structure | 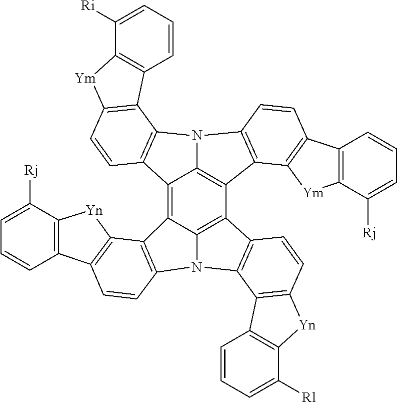 |
| Compound 52- (Ri)(Rj)(Rk)(Rl) (Ym)(Yn), wherein Compound 52- (R1)(R1)(R1)(R1)(Y1) (Y1) to Compound 52- (R468)(R468)(R468) (R468)(Y51)(Y51), have the structure | 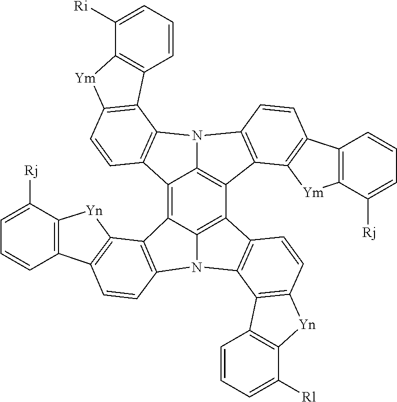 |
| Compound 53- (Ri)(Rj)(Rk)(Rl) (Ym)(Yn), wherein Compound 53- (R1)(R1)(R1)(R1)(Y1) (Y1) to Compound 53- (R468)(R468)(R468) (R468)(Y51)(Y51), have the structure | 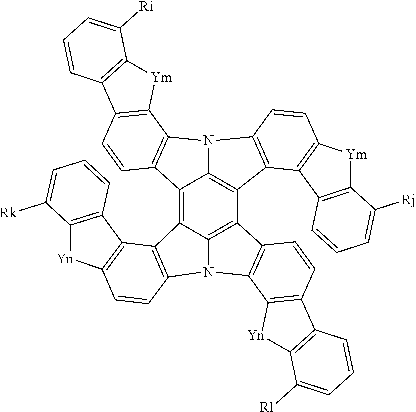 |
| Compound 54- (Ri)(Rj)(Rk)(Rl) (Ym)(Yn), wherein Compound 54- (R1)(R1)(R1)(R1)(Y1) (Y1) to Compound 54- (R468)(R468)(R468) (R468)(Y51)(Y51), have the structure | 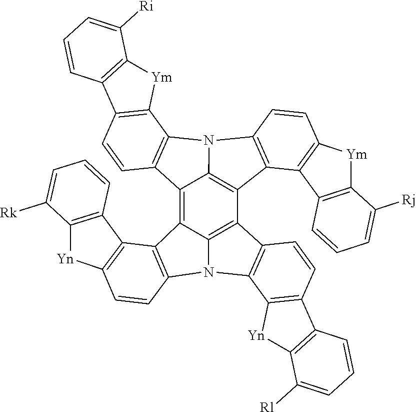 |
| Compound 55- (Ri)(Rj)(Rk)(Rl) (Ym)(Yn), wherein Compound 55- (R1)(R1)(R1)(R1)(Y1) (Y1) to Compound 55- (R468)(R468)(R468) (R468)(Y51)(Y51), have the structure | 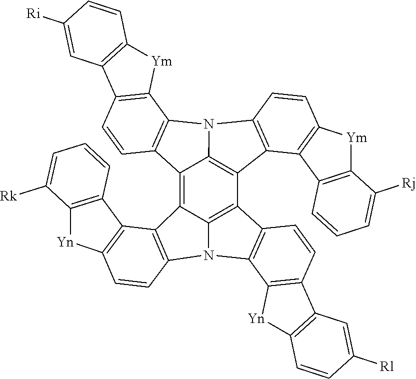 |
| Compound 56- (Ri)(Rj)(Rk)(Rl) (Ym)(Yn), wherein Compound 56- (R1)(R1)(R1)(R1)(Y1) (Y1) to Compound 56- (R468)(R468)(R468) (R468)(Y51)(Y51), have the structure | 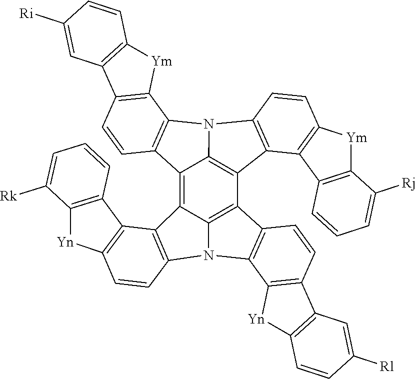 |
| Compound 57- (Ri)(Rj)(Rk)(Rl) (Ym)(Yn), wherein Compound 57- (R1)(R1)(R1)(R1)(Y1) (Y1) to Compound 57- (R468)(R468)(R468) (R468)(Y51)(Y51), have the structure | 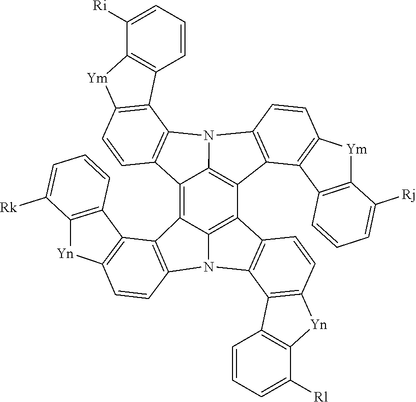 |
| Compound 58- (Ri)(Rj)(Rk)(Rl) (Ym)(Yn), wherein Compound 58- (R1)(R1)(R1)(R1)(Y1) (Y1) to Compound 58- (R468)(R468)(R468) (R468)(Y51)(Y51), have the structure | 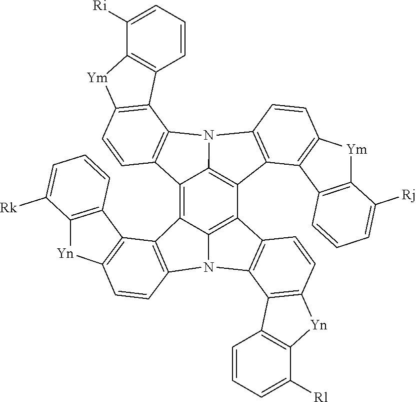 |
| Compound 59- (Ri)(Rj)(Rk)(Rl) (Ym)(Yn), wherein Compound 59- (R1)(R1)(R1)(R1)(Y1) (Y1) to Compound 59- (R468)(R468)(R468) (R468)(Y51)(Y51), have the structure | 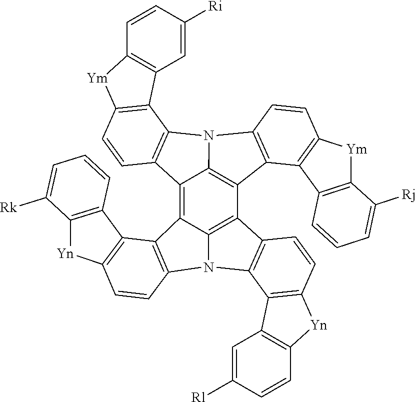 |
| Compound 60- (Ri)(Rj)(Rk)(Rl) (Ym)(Yn), wherein Compound 60- (R1)(R1)(R1)(R1)(Y1) (Y1) to Compound 60- (R468)(R468)(R468) (R468)(Y51)(Y51), have the structure | 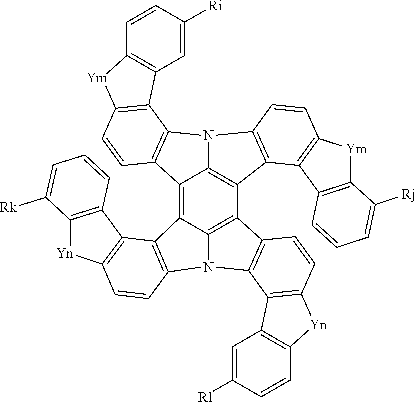 |
| Compound 61- (Ri)(Rj)(Rk)(Rl)(Ym), wherein Compound 61- (R1)(R1)(R1)(R1)(Y1) to Compound 61- (R468)(R468)(R468) (R468)(Y51), have the structure | 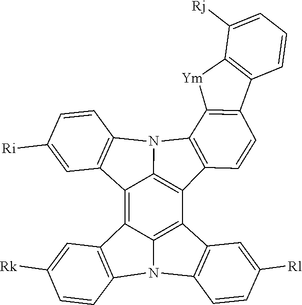 |
| Compound 62- (Ri)(Rj)(Rk)(Rl)(Ym), wherein Compound 62- (R1)(R1)(R1)(R1)(Y1) to Compound 62- (R468)(R468)(R468) (R468)(Y51), have the structure | 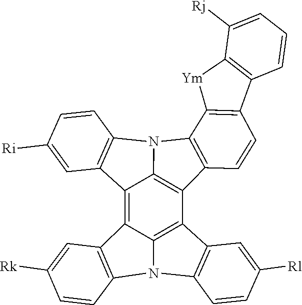 |
| Compound 63- (Ri)(Rj)(Rk)(Rl)(Ym), wherein Compound 63- (R1)(R1)(R1)(R1)(Y1) to Compound 63- (R468)(R468)(R468) (R468)(Y51), have the structure | 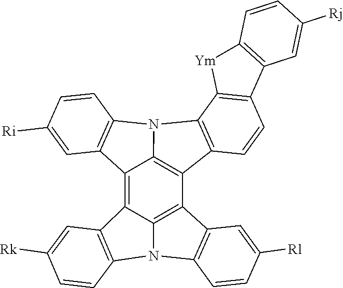 |
| Compound 64- (Ri)(Rj)(Rk)(Rl)(Ym), wherein Compound 64- (R1)(R1)(R1)(R1)(Y1) to Compound 64- (R468)(R468)(R468) (R468)(Y51), have the structure | 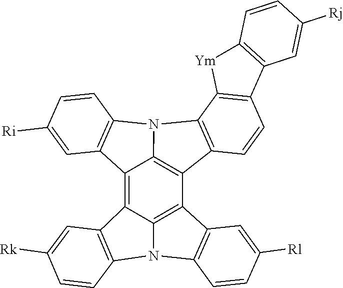 |
| Compound 65- (Ri)(Rj)(Rk)(Rl)(Ym), wherein Compound 65- (R1)(R1)(R1)(R1)(Y1) to Compound 65- (R468)(R468)(R468) (R468)(Y51), have the structure | 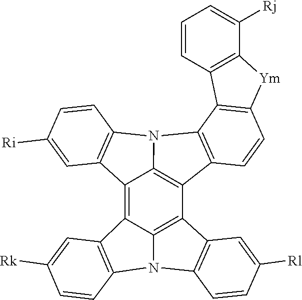 |
| Compound 66- (Ri)(Rj)(Rk)(Rl)(Ym), wherein Compound 66- (R1)(R1)(R1)(R1)(Y1) to Compound 66- (R468)(R468)(R468) (R468)(Y51), have the structure | 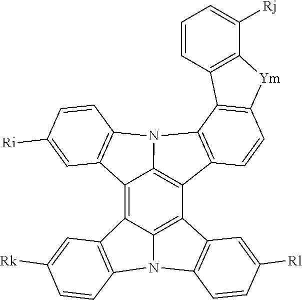 |
| Compound 67- (Ri)(Rj)(Rk)(Rl)(Ym), wherein Compound 67- (R1)(R1)(R1)(R1)(Y1) to Compound 67- (R468)(R468)(R468) (R468)(Y51), have the structure | 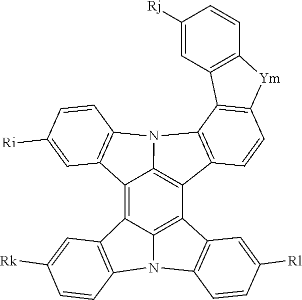 |
| Compound 68- (Ri)(Rj)(Rk)(Rl)(Ym), wherein Compound 68- (R1)(R1)(R1)(R1)(Y1) to Compound 68- (R468)(R468)(R468) (R468)(Y51), have the structure | 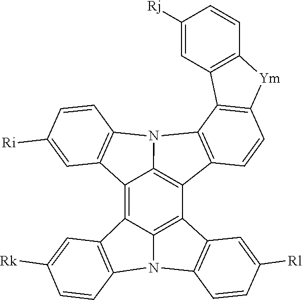 |
| Compound 69- (Ri)(Rj)(Rk)(Rl)(Ym), wherein Compound 69- (R1)(R1)(R1)(R1)(Y1) to Compound 69- (R468)(R468)(R468) (R468)(Y51), have the structure | 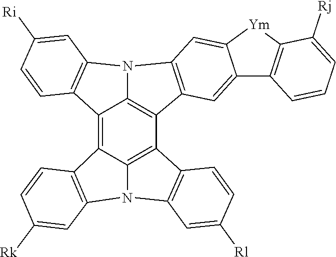 |
| Compound 70- (Ri)(Rj)(Rk)(Rl)(Ym), wherein Compound 70- (R1)(R1)(R1)(R1)(Y1) to Compound 70- (R468)(R468)(R468) (R468)(Y51), have the structure | 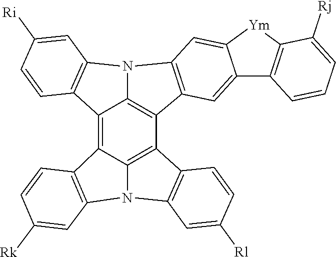 |
| Compound 71- (Ri)(Rj)(Rk)(Rl)(Ym), wherein Compound 71- (R1)(R1)(R1)(R1)(Y1) to Compound 71- (R468)(R468)(R468) (R468)(Y51), have the structure | 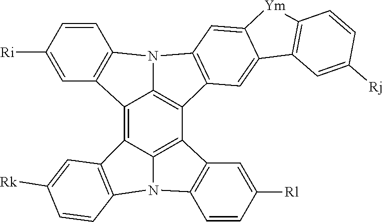 |
| Compound 72- (Ri)(Rj)(Rk)(Rl)(Ym), wherein Compound 72- (R1)(R1)(R1)(R1)(Y1) to Compound 72- (R468)(R468)(R468) (R468)(Y51), have the structure | 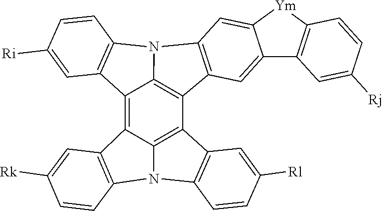 |
| Compound 73- (Ri)(Rj)(Rk)(Rl)(Ym), wherein Compound 73- (R1)(R1)(R1)(R1)(Y1) to Compound 73- (R468)(R468)(R468) (R468)(Y51), have the structure | 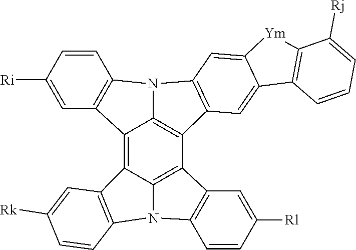 |
| Compound 74- (Ri)(Rj)(Rk)(Rl)(Ym), wherein Compound 74- (R1)(R1)(R1)(R1)(Y1) to Compound 74- (R468)(R468)(R468) (R468)(Y51), have the structure | 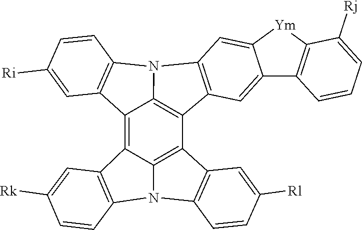 |
| Compound 75- (Ri)(Rj)(Rk)(Rl)(Ym), wherein Compound 75- (R1)(R1)(R1)(R1)(Y1) to Compound 75- (R468)(R468)(R468) (R468)(Y51), have the structure | 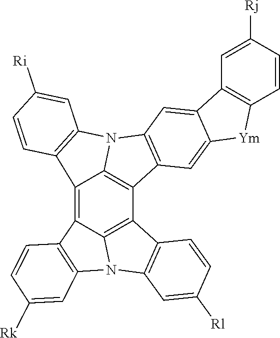 |
| Compound 76- (Ri)(Rj)(Rk)(Rl)(Ym), wherein Compound 76- (R1)(R1)(R1)(R1)(Y1) to Compound 76- (R468)(R468)(R468) (R468)(Y51), have the structure | 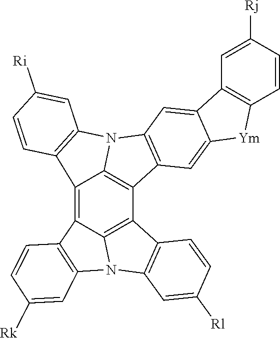 |
| Compound 77- (Ri)(Rj)(Rk)(Rl)(Ym), wherein Compound 77- (R1)(R1)(R1)(R1)(Y1) to Compound 77- (R468)(R468)(R468) (R468)(Y51), have the structure | 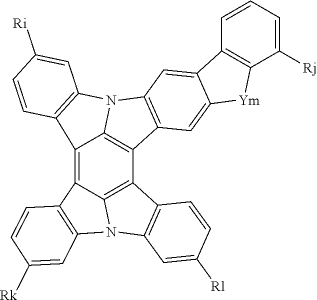 |
| Compound 78- (Ri)(Rj)(Rk)(Rl)(Ym), wherein Compound 78- (R1)(R1)(R1)(R1)(Y1) to Compound 78- (R468)(R468)(R468) (R468)(Y51), have the structure | 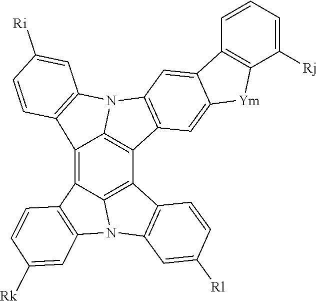 |
| Compound 79- (Ri)(Rj)(Rk)(Rl)(Ym), wherein Compound 79- (R1)(R1)(R1)(R1)(Y1) to Compound 79- (R468)(R468)(R468) (R468)(Y51), have the structure | 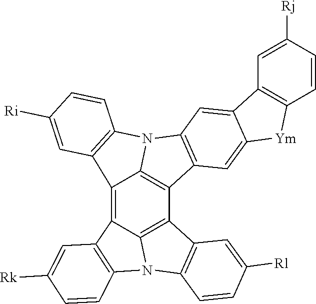 |
| Compound 80- (Ri)(Rj)(Rk)(Rl)(Ym), wherein Compound 80- (R1)(R1)(R1)(R1)(Y1) to Compound 80- (R468)(R468)(R468) (R468)(Y51), have the structure | 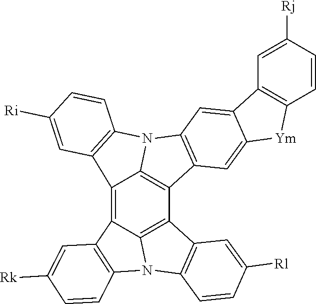 |
| Compound 81- (Ri)(Rj)(Rk)(Rl)(Ym), wherein Compound 81- (R1)(R1)(R1)(R1)(Y1) to Compound 81- (R468)(R468)(R468) (R468)(Y51), have the structure | 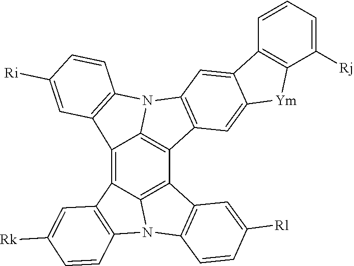 |
| Compound 82- (Ri)(Rj)(Rk)(Rl)(Ym), wherein Compound 82- (R1)(R1)(R1)(R1)(Y1) to Compound 82- (R468)(R468)(R468) (R468)(Y51), have the structure | 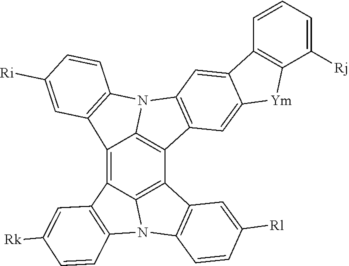 |
| Compound 83- (Ri)(Rj)(Rk)(Rl)(Ym), wherein Compound 83- (R1)(R1)(R1)(R1)(Y1) to Compound 83- (R468)(R468)(R468) (R468)(Y51), have the structure | 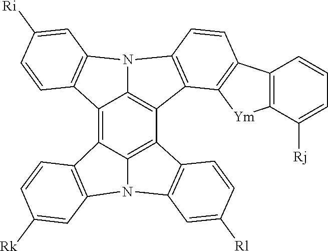 |
| Compound 84- (Ri)(Rj)(Rk)(Rl)(Ym), wherein Compound 84- (R1)(R1)(R1)(R1)(Y1) to Compound 84- (R468)(R468)(R468) (R468)(Y51), have the structure | 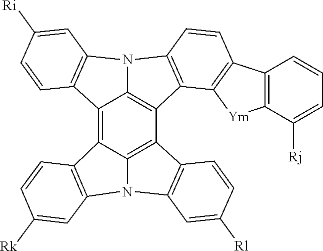 |
| Compound 85- (Ri)(Rj)(Rk)(Rl)(Ym), wherein Compound 85- (R1)(R1)(R1)(R1)(Y1) to Compound 85- (R468)(R468)(R468) (R468)(Y51), have the structure | 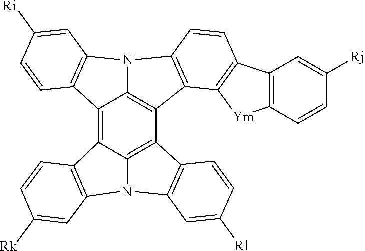 |
| Compound 86- (Ri)(Rj)(Rk)(Rl)(Ym), wherein Compound 86- (R1)(R1)(R1)(R1)(Y1) to Compound 86- (R468)(R468)(R468) (R468)(Y51), have the structure | 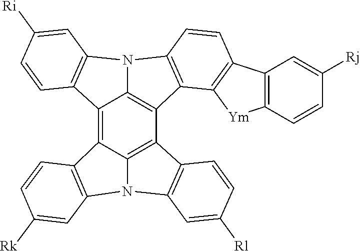 |
| Compound 87- (Ri)(Rj)(Rk)(Rl)(Ym), wherein Compound 87- (R1)(R1)(R1)(R1)(Y1) to Compound 87- (R468)(R468)(R468) (R468)(Y51), have the structure | 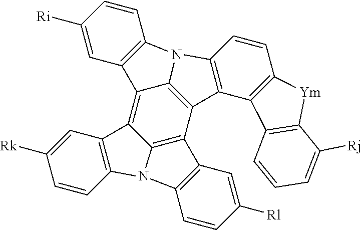 |
| Compound 88- (Ri)(Rj)(Rk)(Rl)(Ym), wherein Compound 88- (R1)(R1)(R1)(R1)(Y1) to Compound 88- (R468)(R468)(R468) (R468)(Y51), have the structure | 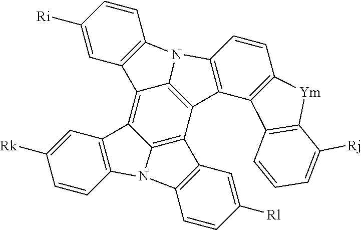 |
| Compound 89- (Ri)(Rj)(Rk)(Rl)(Ym), wherein Compound 89- (R1)(R1)(R1)(R1)(Y1) to Compound 89- (R468)(R468)(R468) (R468)(Y51), have the structure | 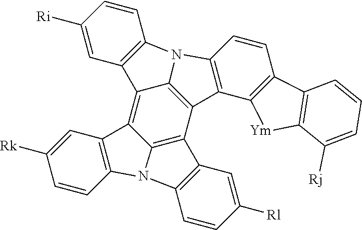 |
| Compound 90- (Ri)(Rj)(Rk)(Rl)(Ym), wherein Compound 90- (R1)(R1)(R1)(R1)(Y1) to Compound 90- (R468)(R468)(R468) (R468)(Y51), have the structure | 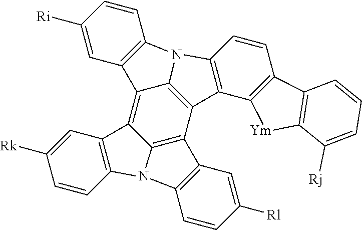 |
| Compound 91- (Ri)(Rj)(Rk)(Rl)(Ym), wherein Compound 91- (R1)(R1)(R1)(R1)(Y1) to Compound 91- (R468)(R468)(R468) (R468)(Y51), have the structure | 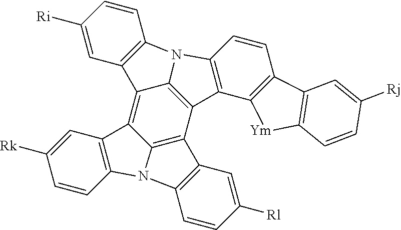 |
| Compound 92- (Ri)(Rj)(Rk)(Rl)(Ym), wherein Compound 92- (R1)(R1)(R1)(R1)(Y1) to Compound 92- (R468)(R468)(R468) (R468)(Y51), have the structure | 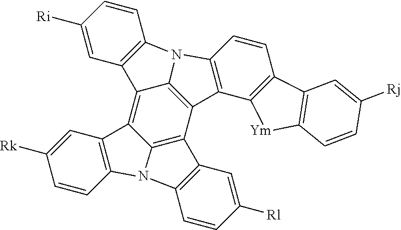 |
| Compound 93- (Ri)(Rj)(Rk)(Rl) (Ym)(Yn), wherein Compound 93- (R1)(R1)(R1)(R1) (Y1)(Y1) to Compound 93- (R468)(R468)(R468) (R468)(Y51)(Y51), have the structure | 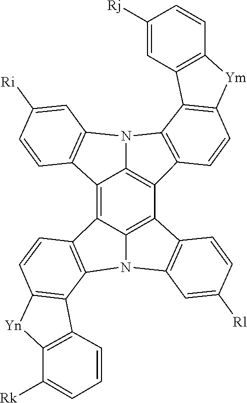 |
| Compound 94- (Ri)(Rj)(Rk)(Rl) (Ym)(Yn), wherein Compound 94- (R1)(R1)(R1)(R1) (Y1)(Y1) to Compound 94- (R468)(R468)(R468) (R468)(Y51)(Y51), have the structure | 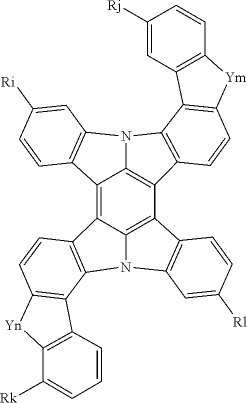 |
| Compound 95- (Ri)(Rj)(Rk)(Rl) (Ym)(Yn), wherein Compound 95- (R1)(R1)(R1)(R1) (Y1)(Y1) to Compound 95- (R468)(R468)(R468) (R468)(Y51)(Y51), have the structure | 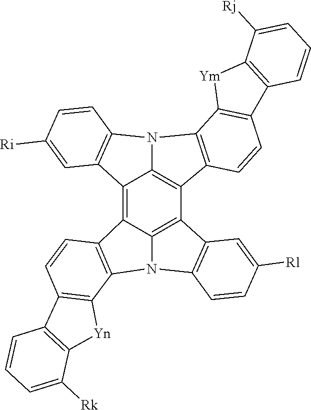 |
| Compound 96- (Ri)(Rj)(Rk)(Rl) (Ym)(Yn), wherein Compound 96- (R1)(R1)(R1)(R1) (Y1)(Y1) to Compound 96- (R468)(R468)(R468) (R468)(Y51)(Y51), have the structure | 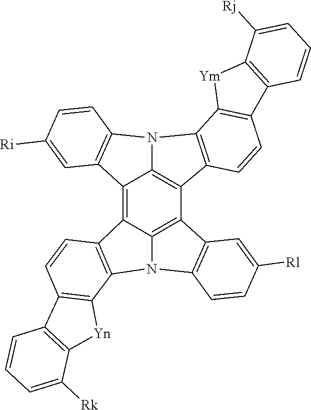 |
| Compound 97- (Ri)(Rj)(Rk)(Rl) (Ym)(Yn), wherein Compound 97- (R1)(R1)(R1)(R1) (Y1)(Y1) to Compound 97- (R468)(R468)(R468) (R468)(Y51)(Y51), have the structure | 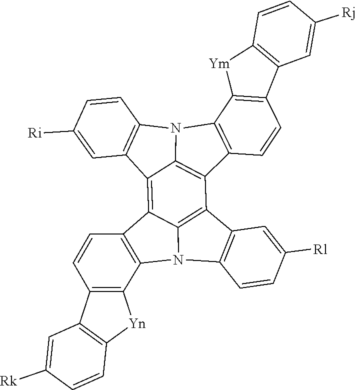 |
| Compound 98- (Ri)(Rj)(Rk)(Rl) (Ym)(Yn), wherein Compound 98- (R1)(R1)(R1)(R1) (Y1)(Y1) to Compound 98- (R468)(R468)(R468) (R468)(Y51)(Y51), have the structure | 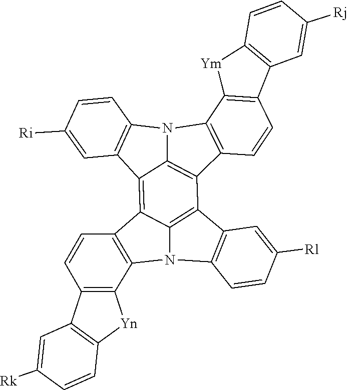 |
| Compound 99- (Ri)(Rj)(Rk)(Rl) (Ym)(Yn), wherein Compound 99- (R1)(R1)(R1)(R1) (Y1)(Y1) to Compound 99- (R468)(R468)(R468) (R468)(Y51)(Y51), have the structure | 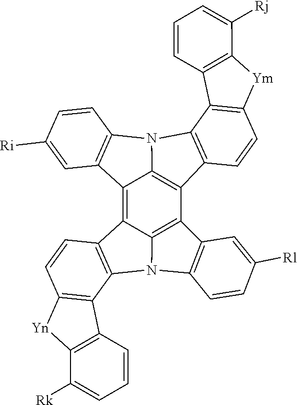 |
| Compound 100- (Ri)(Rj)(Rk)(Rl) (Ym)(Yn), wherein Compound 100- (R1)(R1)(R1)(R1) (Y1)(Y1) to Compound 100- (R468)(R468)(R468) (R468)(Y51)(Y51), have the structure | 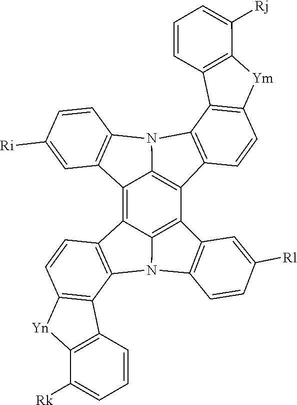 |
| Compound 101- (Ri)(Rj)(Rk)(Rl) (Ym)(Yn), wherein Compound 101- (R1)(R1)(R1)(R1) (Y1)(Y1) to Compound 101- (R468)(R468)(R468) (R468)(Y51)(Y51), have the structure | 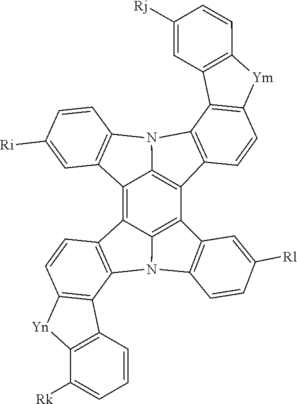 |
| Compound 102- (Ri)(Rj)(Rk)(Rl) (Ym)(Yn), wherein Compound 102- (R1)(R1)(R1)(R1) (Y1)(Y1) to Compound 102- (R468)(R468)(R468) (R468)(Y51)(Y51), have the structure | 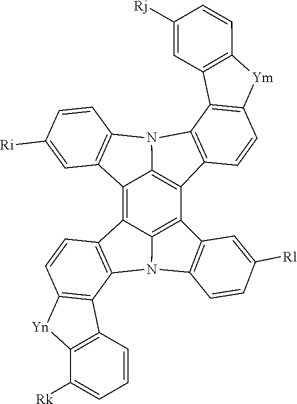 |
| Compound 103- (Ri)(Rj)(Rk)(Rl) (Ym)(Yn), wherein Compound 103- (R1)(R1)(R1)(R1) (Y1)(Y1) to Compound 103- (R468)(R468)(R468) (R468)(Y51)(Y51), have the structure | 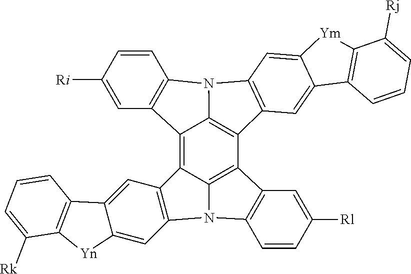 |
| Compound 104- (Ri)(Rj)(Rk)(Rl) (Ym)(Yn), wherein Compound 104- (R1)(R1)(R1)(R1) (Y1)(Y1) to Compound 104- (R468)(R468)(R468) (R468)(Y51)(Y51), have the structure | 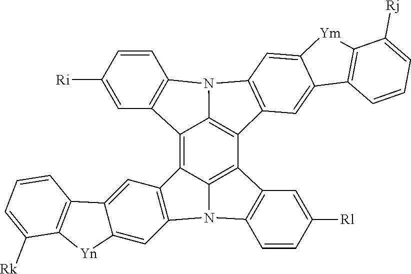 |
| Compound 105- (Ri)(Rj)(Rk)(Rl) (Ym)(Yn), wherein Compound 105- (R1)(R1)(R1)(R1) (Y1)(Y1) to Compound 105- (R468)(R468)(R468) (R468)(Y51)(Y51), have the structure | 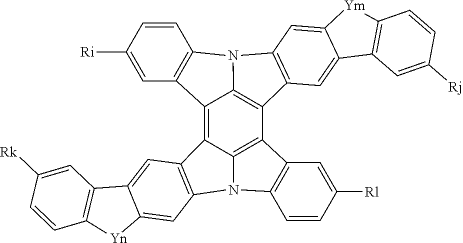 |
| Compound 106- (Ri)(Rj)(Rk)(Rl) (Ym)(Yn), wherein Compound 106- (R1)(R1)(R1)(R1) (Y1)(Y1) to Compound 106- (R468)(R468)(R468) (R468)(Y51)(Y51), have the structure | 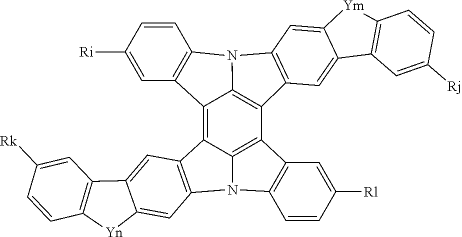 |
| Compound 107- (Ri)(Rj)(Rk)(Rl) (Ym)(Yn), wherein Compound 107- (R1)(R1)(R1)(R1) (Y1)(Y1) to Compound 107- (R468)(R468)(R468) (R468)(Y51)(Y51), have the structure | 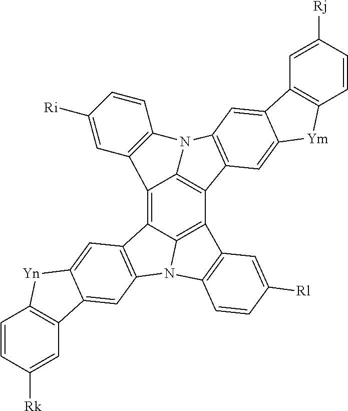 |
| Compound 108- (Ri)(Rj)(Rk)(Rl) (Ym)(Yn), wherein Compound 108- (R1)(R1)(R1)(R1) (Y1)(Y1) to Compound 108- (R468)(R468)(R468) (R468)(Y51)(Y51), have the structure | 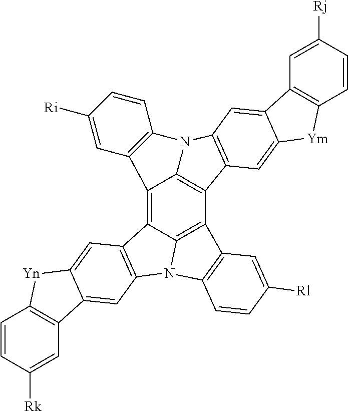 |
| Compound 109- (Ri)(Rj)(Rk)(Rl) (Ym)(Yn), wherein Compound 109- (R1)(R1)(R1)(R1) (Y1)(Y1) to Compound 109- (R468)(R468)(R468) (R468)(Y51)(Y51), have the structure | 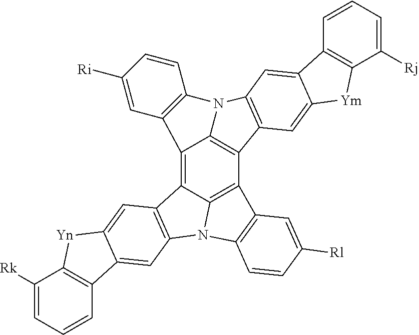 |
| Compound 110- (Ri)(Rj)(Rk)(Rl) (Ym)(Yn), wherein Compound 110- (R1)(R1)(R1)(R1) (Y1)(Y1) to Compound 110- (R468)(R468)(R468) (R468)(Y51)(Y51), have the structure | 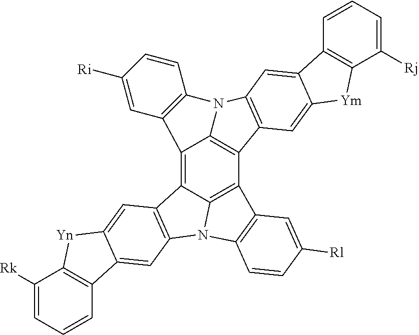 |
| Compound 111- (Ri)(Rj)(Rk)(Rl) (Ym)(Yn), wherein Compound 111- (R1)(R1)(R1)(R1) (Y1)(Y1) to Compound 111- (R468)(R468)(R468) (R468)(Y51)(Y51), have the structure | 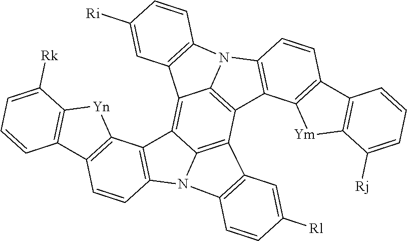 |
| Compound 112- (Ri)(Rj)(Rk)(Rl) (Ym)(Yn), wherein Compound 112- (R1)(R1)(R1)(R1) (Y1)(Y1) to Compound 112- (R468)(R468)(R468) (R468)(Y51)(Y51), have the structure | 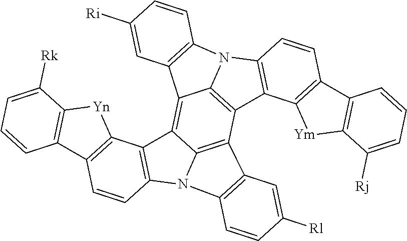 |
| Compound 113- (Ri)(Rj)(Rk)(Rl) (Ym)(Yn), wherein Compound 113- (R1)(R1)(R1)(R1) (Y1)(Y1) to Compound 113- (R468)(R468)(R468) (R468)(Y51)(Y51), have the structure | 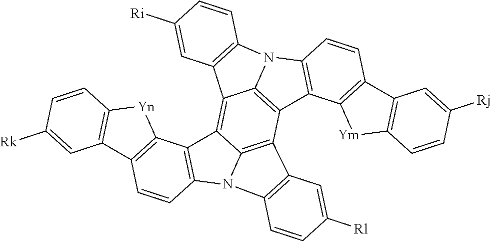 |
| Compound 114- (Ri)(Rj)(Rk)(Rl) (Ym)(Yn), wherein Compound 114- (R1)(R1)(R1)(R1) (Y1)(Y1) to Compound 114- (R468)(R468)(R468) (R468)(Y51)(Y51), have the structure | 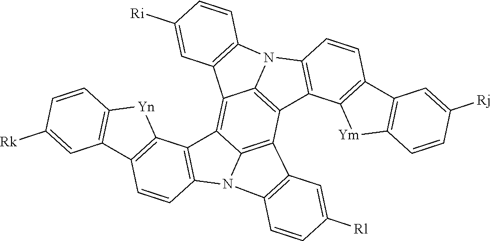 |
| Compound 115- (Ri)(Rj)(Rk)(Rl) (Ym)(Yn), wherein Compound 115- (R1)(R1)(R1)(R1) (Y1)(Y1) to Compound 115- (R468)(R468)(R468) (R468)(Y51)(Y51), have the structure | 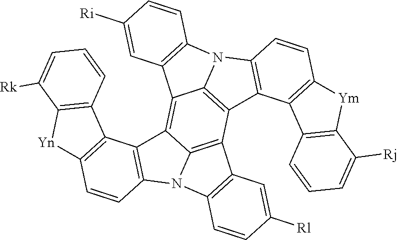 |
| Compound 116- (Ri)(Rj)(Rk)(Rl) (Ym)(Yn), wherein Compound 116- (R1)(R1)(R1)(R1) (Y1)(Y1) to Compound 116- (R468)(R468)(R468) (R468)(Y51)(Y51), have the structure | 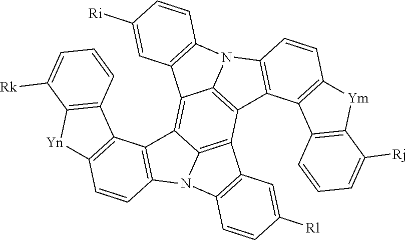 |
| Compound 117- (Ri)(Rj)(Rk)(Rl) (Ym)(Yn), wherein Compound 117- (R1)(R1)(R1)(R1) (Y1)(Y1) to Compound 117- (R468)(R468)(R468) (R468)(Y51)(Y51), have the structure | 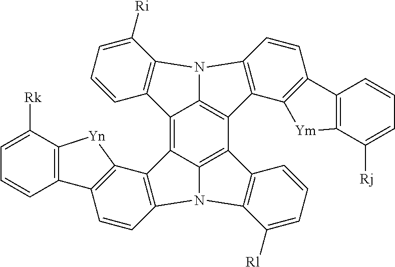 |
| Compound 118- (Ri)(Rj)(Rk)(Rl) (Ym)(Yn), wherein Compound 118- (R1)(R1)(R1)(R1) (Y1)(Y1) to Compound 118- (R468)(R468)(R468) (R468)(Y51)(Y51), have the structure | 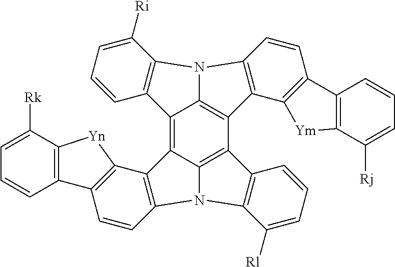 |
| Compound 119- (Ri)(Rj)(Rk)(Rl) (Ym)(Yn), wherein Compound 119- (R1)(R1)(R1)(R1) (Y1)(Y1) to Compound 119- (R468)(R468)(R468) (R468)(Y51)(Y51), have the structure | 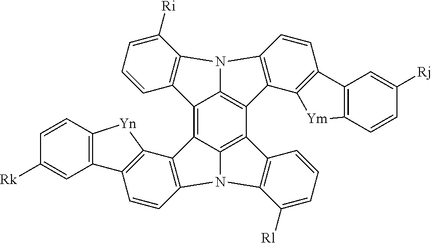 |
| Compound 120- (Ri)(Rj)(Rk)(Rl) (Ym)(Yn), wherein Compound 120- (R1)(R1)(R1)(R1) (Y1)(Y1) to Compound 120- (R468)(R468)(R468) (R468)(Y51)(Y51), have the structure | 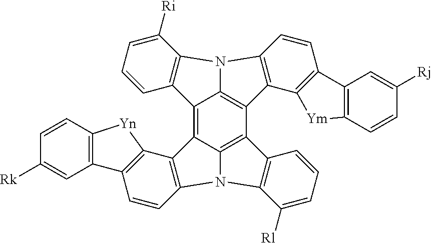 |
| Compound 121- (Ri)(Rj)(Rk)(Rl) (Ym)(Yn), wherein Compound 121- (R1)(R1)(R1)(R1) (Y1)(Y1) to Compound 121- (R468)(R468)(R468) (R468)(Y51)(Y51), have the structure | 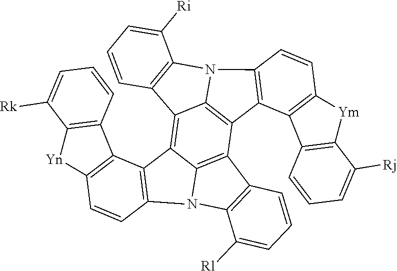 |
| Compound 122- (Ri)(Rj)(Rk)(Rl) (Ym)(Yn), wherein Compound 122- (R1)(R1)(R1)(R1) (Y1)(Y1) to Compound 122- (R468)(R468)(R468) (R468)(Y51)(Y51), have the structure | 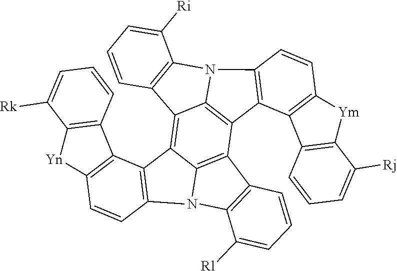 |
| Compound 123- (Ri)(Rj)(Rk)(Rl) (Ym)(Yn), wherein Compound 123- (R1)(R1)(R1)(R1) (Y1)(Y1) to Compound 123- (R468)(R468)(R468) (R468)(Y51)(Y51), have the structure | 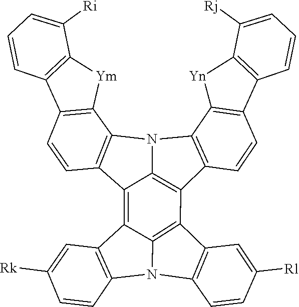 |
| Compound 124- (Ri)(Rj)(Rk)(Rl) (Ym)(Yn), wherein Compound 124- (R1)(R1)(R1)(R1) (Y1)(Y1) to Compound 125- (R468)(R468)(R468) (R468)(Y51)(Y51), have the structure | 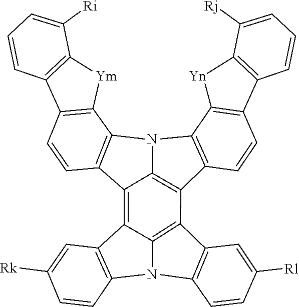 |
| Compound 125- (Ri)(Rj)(Rk)(Rl) (Ym)(Yn), wherein Compound 125- (R1)(R1)(R1)(R1) (Y1)(Y1) to Compound 125- (R468)(R468)(R468) (R468)(Y51)(Y51), have the structure | 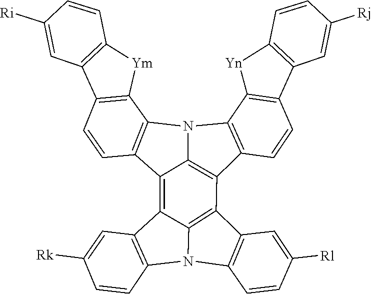 |
| Compound 126- (Ri)(Rj)(Rk)(Rl) (Ym)(Yn), wherein Compound 126- (R1)(R1)(R1)(R1) (Y1)(Y1) to Compound 126- (R468)(R468)(R468) (R468)(Y51)(Y51), have the structure | 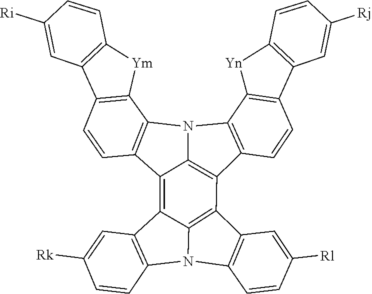 |
| Compound 127- (Ri)(Rj)(Rk)(Rl) (Ym)(Yn), wherein Compound 127- (R1)(R1)(R1)(R1) (Y1)(Y1) to Compound 127- (R468)(R468)(R468) (R468)(Y51)(Y51), have the structure | 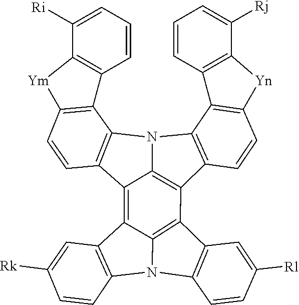 |
| Compound 128- (Ri)(Rj)(Rk)(Rl) (Ym)(Yn), wherein Compound 128- (R1)(R1)(R1)(R1) (Y1)(Y1) to Compound 128- (R468)(R468)(R468) (R468)(Y51)(Y51), have the structure | 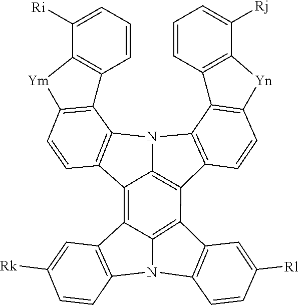 |
| Compound 129- (Ri)(Rj)(Rk)(Rl) (Ym)(Yn), wherein Compound 129- (R1)(R1)(R1)(R1) (Y1)(Y1) to Compound 129- (R468)(R468)(R468) (R468)(Y51)(Y51), have the structure | 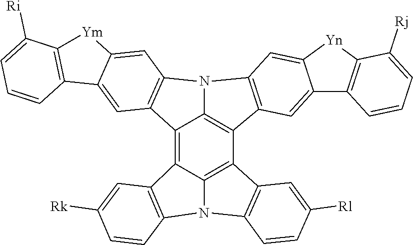 |
| Compound 130- (Ri)(Rj)(Rk)(Rl) (Ym)(Yn), wherein Compound 130- (R1)(R1)(R1)(R1) (Y1)(Y1) to Compound 130- (R468)(R468)(R468) (R468)(Y51)(Y51), have the structure | 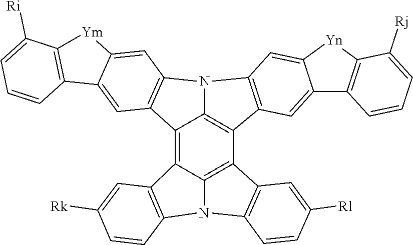 |
| Compound 131- (Ri)(Rj)(Rk)(Rl) (Ym)(Yn), wherein Compound 131- (R1)(R1)(R1)(R1) (Y1)(Y1) to Compound 131- (R468)(R468)(R468) (R468)(Y51)(Y51), have the structure | 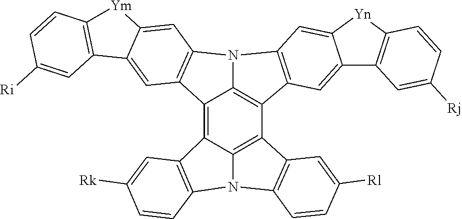 |
| Compound 132- (Ri)(Rj)(Rk)(Rl) (Ym)(Yn), wherein Compound 132- (R1)(R1)(R1)(R1) (Y1)(Y1) to Compound 132- (R468)(R468)(R468) (R468)(Y51)(Y51), have the structure | 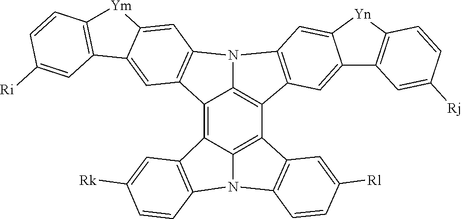 |
| Compound 133- (Ri)(Rj)(Rk)(Rl) (Ym)(Yn), wherein Compound 133- (R1)(R1)(R1)(R1) (Y1)(Y1) to Compound 133- (R468)(R468)(R468) (R468)(Y51)(Y51), have the structure | 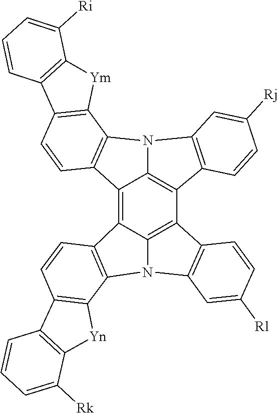 |
| Compound 134- (Ri)(Rj)(Rk)(Rl) (Ym)(Yn), wherein Compound 134- (R1)(R1)(R1)(R1) (Y1)(Y1) to Compound 134- (R468)(R468)(R468) (R468)(Y51)(Y51), have the structure | 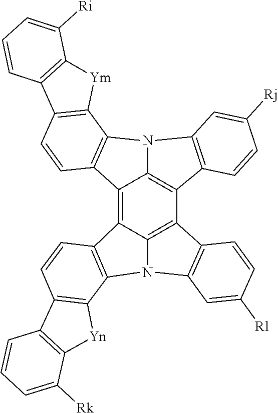 |
| Compound 135- (Ri)(Rj)(Rk)(Rl) (Ym)(Yn), wherein Compound 135- (R1)(R1)(R1)(R1) (Y1)(Y1) to Compound 135- (R468)(R468)(R468) (R468)(Y51)(Y51), have the structure | 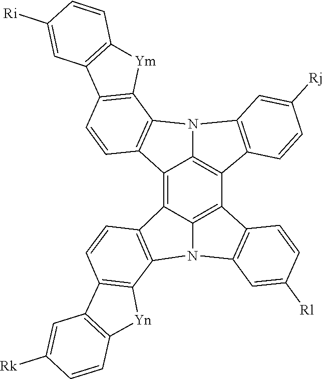 |
| Compound 136- (Ri)(Rj)(Rk)(Rl) (Ym)(Yn), wherein Compound 136- (R1)(R1)(R1)(R1) (Y1)(Y1) to Compound 136- (R468)(R468)(R468) (R468)(Y51)(Y51), have the structure | 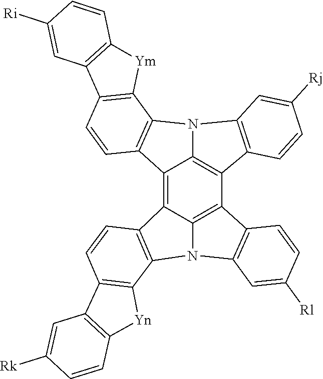 |
| Compound 137- (Ri)(Rj)(Rk)(Rl) (Ym)(Yn), wherein Compound 137- (R1)(R1)(R1)(R1) (Y1)(Y1) to Compound 137- (R468)(R468)(R468) (R468)(Y51)(Y51), have the structure | 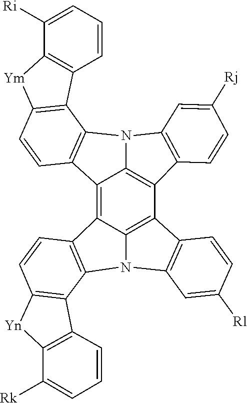 |
| Compound 138- (Ri)(Rj)(Rk)(Rl) (Ym)(Yn), wherein Compound 138- (R1)(R1)(R1)(R1) (Y1)(Y1) to Compound 138- (R468)(R468)(R468) (R468)(Y51)(Y51), have the structure | 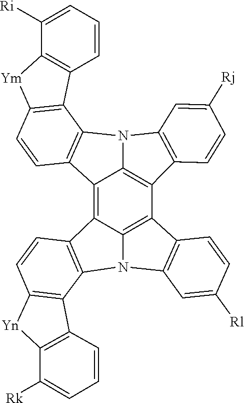 |
| Compound 139- (Ri)(Rj)(Rk)(Rl) (Ym)(Yn), wherein Compound 139- (R1)(R1)(R1)(R1) (Y1)(Y1) to Compound 139- (R468)(R468)(R468) (R468)(Y51)(Y51), have the structure | 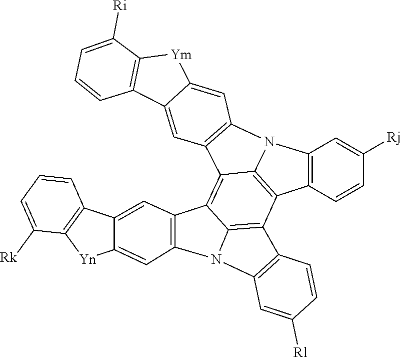 |
| Compound 140- (Ri)(Rj)(Rk)(Rl) (Ym)(Yn), wherein Compound 140- (R1)(R1)(R1)(R1) (Y1)(Y1) to Compound 140- (R468)(R468)(R468) (R468)(Y51)(Y51), have the structure | 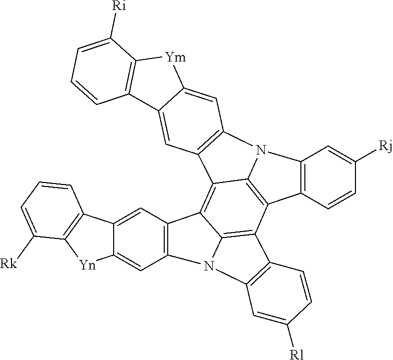 |
| Compound 141- (Ri)(Rj)(Rk)(Rl) (Ym)(Yn), wherein Compound 141- (R1)(R1)(R1)(R1) (Y1)(Y1) to Compound 141- (R468)(R468)(R468) (R468)(Y51)(Y51), have the structure | 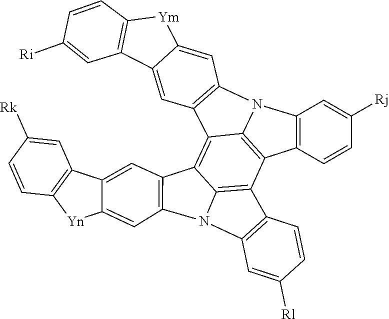 |
| Compound 142- (Ri)(Rj)(Rk)(Rl) (Ym)(Yn), wherein Compound 142- (R1)(R1)(R1)(R1) (Y1)(Y1) to Compound 142- (R468)(R468)(R468) (R468)(Y51)(Y51), have the structure | 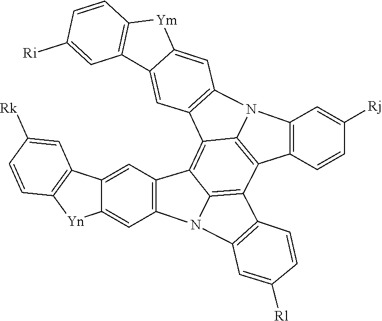 |
| Compound 143- (Ri)(Rj)(Rk)(Rl) (Ym)(Yn), wherein Compound 143- (R1)(R1)(R1)(R1) (Y1)(Y1) to Compound 143- (R468)(R468)(R468) (R468)(Y51)(Y51), have the structure | 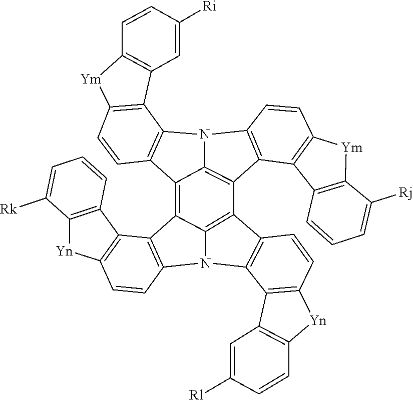 |
| Compound 144- (Ri)(Rj)(Rk)(Rl) (Ym)(Yn), wherein Compound 144- (R1)(R1)(R1)(R1) (Y1)(Y1) to Compound 144- (R468)(R468)(R468) (R468)(Y51)(Y51), have the structure | 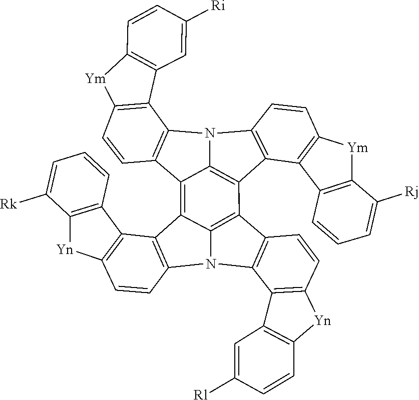 |
| Compound 145- (Ri)(Rk)(Rl)(Rq), wherein Compound 145- (R1)(R1)(R1)(R1) to Compound 145- (R468)(R468)(R468) (R177), have the structure | 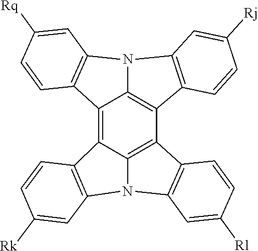 |
| Compound 146- (Ri)(Rk)(Rl)(Rq), wherein Compound 146- (R1)(R1)(R1)(R1) to Compound 146- (R468)(R468)(R468) (R177), have the structure | 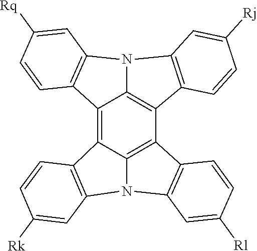 |
| Compound 147- (Ri)(Rk)(Rl)(Rq), wherein Compound 147- (R1)(R1)(R1)(R1) to Compound 147- (R468)(R468)(R468) (R177), have the structure | 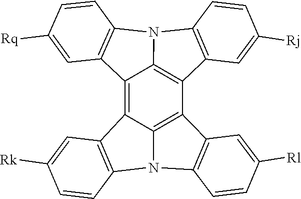 |
| Compound 148- (Ri)(Rk)(Rl)(Rq), wherein Compound 148- (R1)(R1)(R1)(R1) to Compound 148- (R468)(R468)(R468) (R177), have the structure | 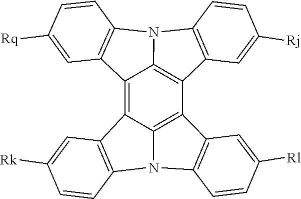 |
| Compound 149- (Ri)(Rk)(Rl)(Rq), wherein Compound 149- (R1)(R1)(R1)(R1) to Compound 149- (R468)(R468)(R468) (R177), have the structure | 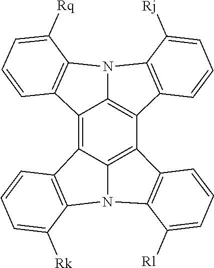 |
| Compound 150- (Ri)(Rk)(Rl)(Rq), wherein Compound 150- (R1)(R1)(R1)(R1) to Compound 150- (R468)(R468)(R468) (R177), have the structure | 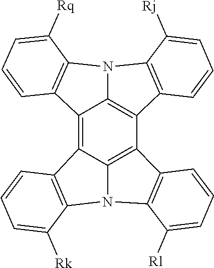 |
| Compound 151- (Ri)(Rk)(Rl)(Rq), wherein Compound 151- (R1)(R1)(R1)(R1) to Compound 151- (R468)(R468)(R468) (R177), have the structure | 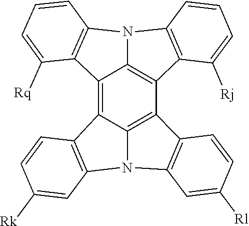 |
| Compound 152- (Ri)(Rk)(Rl)(Rq), wherein Compound 152- (R1)(R1)(R1)(R1) to Compound 152- (R468)(R468)(R468) (R177), have the structure | 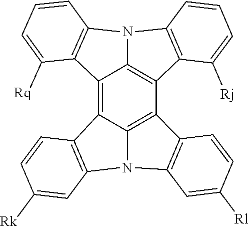 |
| Compound 153- (Ri)(Rk)(Rl)(Rq), wherein Compound 153- (R1)(R1)(R1)(R1) to Compound 153- (R468)(R468)(R468) (R177), have the structure | 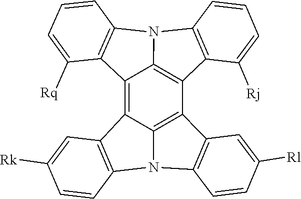 |
| Compound 154- (Ri)(Rk)(Rl)(Rq), wherein Compound 154- (R1)(R1)(R1)(R1) to Compound 154- (R468)(R468)(R468) (R177), have the structure | 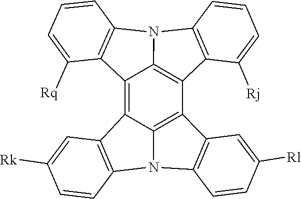 |
| Compound 155- (Rj)(Rk)(Rl)(Rr), wherein Compound 155- (R1)(R1)(R1)(R1) to Compound 155- (R468)(R468)(R468) (R232), have the structure | 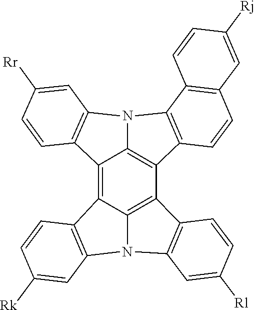 |
| Compound 156- (Rj)(Rk)(Rl)(Rr), wherein Compound 156- (R1)(R1)(R1)(R1) to Compound 156- (R468)(R468)(R468) (R232), have the structure | 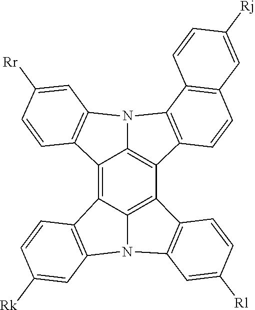 |
| Compound 157- (Rj)(Rk)(Rl)(Rr), wherein Compound 157- (R1)(R1)(R1)(R1) to Compound 157- (R468)(R468)(R468) (R232), have the structure | 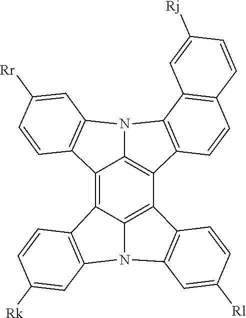 |
| Compound 158- (Rj)(Rk)(Rl)(Rr), wherein Compound 158- (R1)(R1)(R1)(R1) to Compound 158- (R468)(R468)(R468) (R232), have the structure | 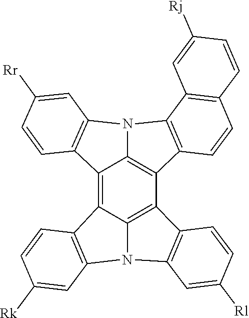 |
| Compound 159- (Rj)(Rk)(Rl)(Rr), wherein Compound 159- (R1)(R1)(R1)(R1) to Compound 159- (R468)(R468)(R468) (R232), have the structure | 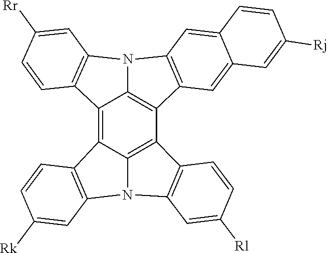 |
| Compound 160- (Rj)(Rk)(Rl)(Rr), wherein Compound 160- (R1)(R1)(R1)(R1) to Compound 160- (R468)(R468)(R468) (R232), have the structure | 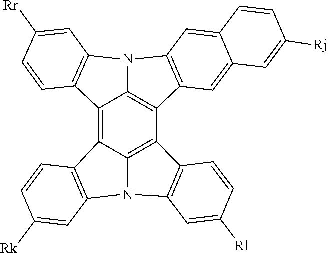 |
| Compound 161- (Rj)(Rk)(Rl)(Rr), wherein Compound 161- (R1)(R1)(R1)(R1) to Compound 161- (R468)(R468)(R468) (R232), have the structure | 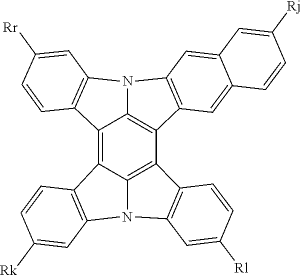 |
| Compound 162- (Rj)(Rk)(Rl)(Rr), wherein Compound 162- (R1)(R1)(R1)(R1) to Compound 162- (R468)(R468)(R468) (R232), have the structure | 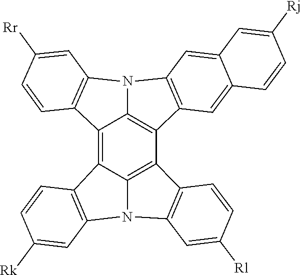 |
| Compound 163- (Rj)(Rk)(Rl)(Rr), wherein Compound 163- (R1)(R1)(R1)(R1) to Compound 163- (R468)(R468)(R468) (R232), have the structure | 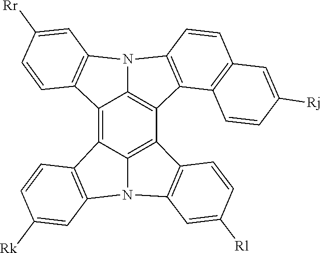 |
| Compound 164- (Rj)(Rk)(Rl)(Rr), wherein Compound 164- (R1)(R1)(R1)(R1) to Compound 164- (R468)(R468)(R468) (R232), have the structure | 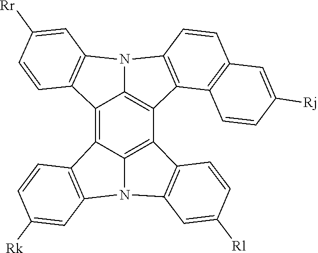 |
| Compound 165- (Rj)(Rk)(Rl)(Rr), wherein Compound 165- (R1)(R1)(R1)(R1) to Compound 165- (R468)(R468)(R468) (R232), have the structure | 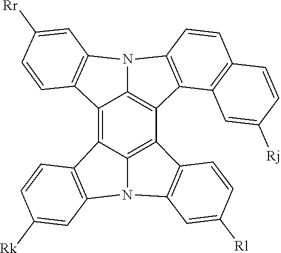 |
| Compound 166- (Rj)(Rk)(Rl)(Rr), wherein Compound 166- (R1)(R1)(R1)(R1) to Compound 166- (R468)(R468)(R468) (R232), have the structure | 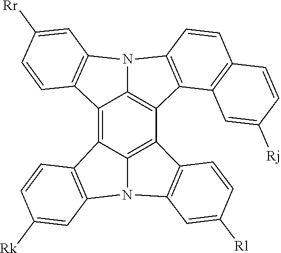 |
| Compound 167- (Rj)(Rk)(Rl)(Rr), wherein Compound 167- (R1)(R1)(R1)(R1) to Compound 167- (R468)(R468)(R468) (R232), have the structure | 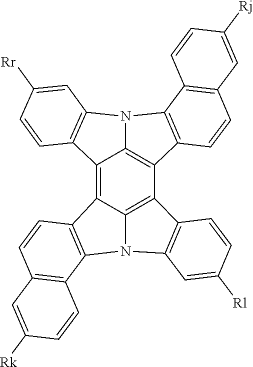 |
| Compound 168- (Rj)(Rk)(Rl)(Rr), wherein Compound 168- (R1)(R1)(R1)(R1) to Compound 168- (R468)(R468)(R468) (R232), have the structure | 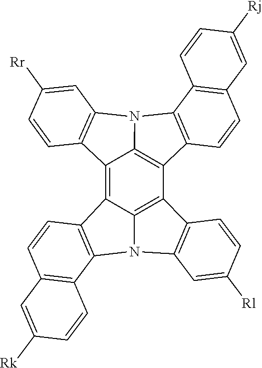 |
| Compound 169- (Rj)(Rk)(Rl)(Rr), wherein Compound 169- (R1)(R1)(R1)(R1) to Compound 169- (R468)(R468)(R468) (R232), have the structure | 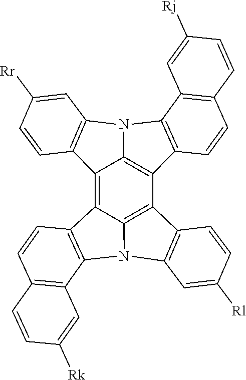 |
| Compound 170- (Rj)(Rk)(Rl)(Rr), wherein Compound 170- (R1)(R1)(R1)(R1) to Compound 170- (R468)(R468)(R468) (R232), have the structure | 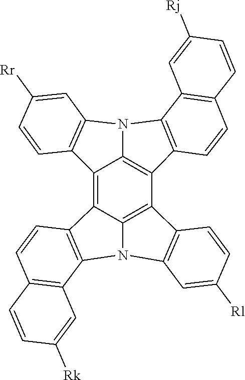 |
| Compound 171- (Rj)(Rk)(Rl)(Rr), wherein Compound 171- (R1)(R1)(R1)(R1) to Compound 171- (R468)(R468)(R468) (R232), have the structure | 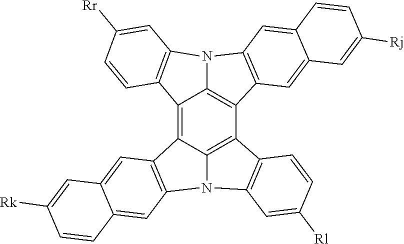 |
| Compound 172- (Rj)(Rk)(Rl)(Rr), wherein Compound 172- (R1)(R1)(R1)(R1) to Compound 172- (R468)(R468)(R468) (R232), have the structure | 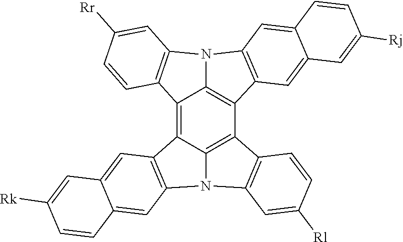 |
| Compound 173- (Rj)(Rk)(Rl)(Rr), wherein Compound 173- (R1)(R1)(R1)(R1) to Compound 173- (R468)(R468)(R468) (R232), have the structure | 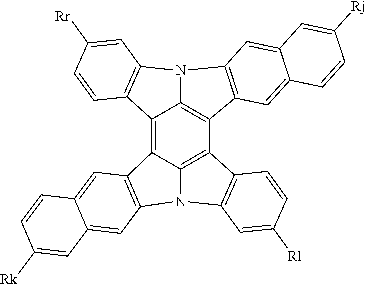 |
| Compound 174- (Rj)(Rk)(Rl)(Rr), wherein Compound 174- (R1)(R1)(R1)(R1) to Compound 174- (R468)(R468)(R468) (R232), have the structure | 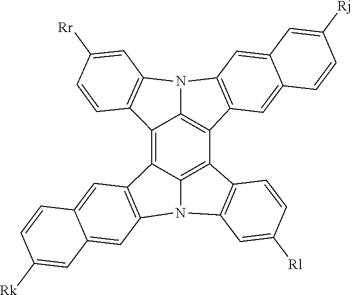 |
| Compound 175- (Rj)(Rk)(Rl)(Rr), wherein Compound 175- (R1)(R1)(R1)(R1) to Compound 175- (R468)(R468)(R468) (R232), have the structure | 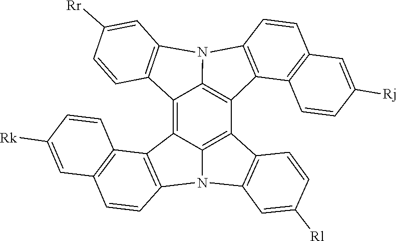 |
| Compound 176- (Rj)(Rk)(Rl)(Rr), wherein Compound 176- (R1)(R1)(R1)(R1) to Compound 176- (R468)(R468)(R468) (R232), have the structure | 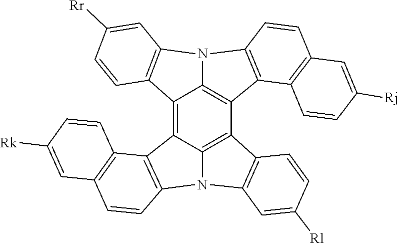 |
| Compound 177- (Rj)(Rk)(Rl)(Rr), wherein Compound 177- (R1)(R1)(R1)(R1) to Compound 177- (R468)(R468)(R468) (R232), have the structure | 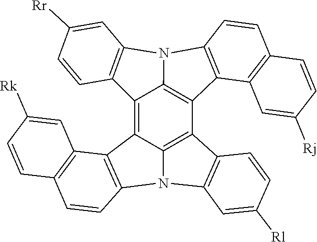 |
| Compound 178- (Rj)(Rk)(Rl)(Rr), wherein Compound 178- (R1)(R1)(R1)(R1) to Compound 178- (R468)(R468)(R468) (R232), have the structure | 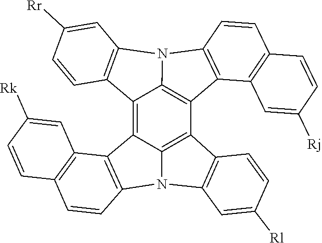 |
| Compound 179- (Rj)(Rk)(Rl)(Rr), wherein Compound 179- (R1)(R1)(R1)(R1) to Compound 179- (R468)(R468)(R468) (R232), have the structure | 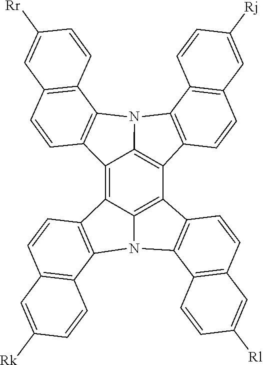 |
| Compound 180- (Rj)(Rk)(Rl)(Rr), wherein Compound 180- (R1)(R1)(R1)(R1) to Compound 180- (R468)(R468)(R468) (R232), have the structure | 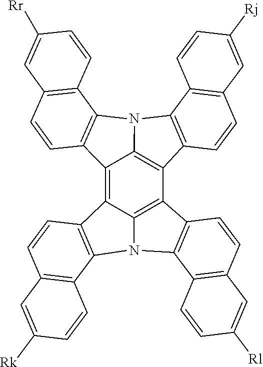 |
| Compound 181- (Rj)(Rk)(Rl)(Rr), wherein Compound 181- (R1)(R1)(R1)(R1) to Compound 181- (R468)(R468)(R468) (R232), have the structure | 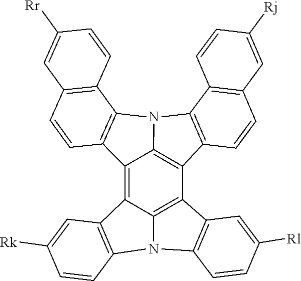 |
| Compound 182- (Rj)(Rk)(Rl)(Rr), wherein Compound 182- (R1)(R1)(R1)(R1) to Compound 182- (R468)(R468)(R468) (R232), have the structure | 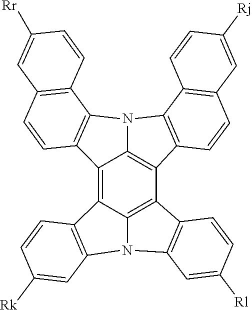 |
| Compound 183- (Rj)(Rk)(Rl)(Rr), wherein Compound 183- (R1)(R1)(R1)(R1) to Compound 183- (R468)(R468)(R468) (R232), have the structure | 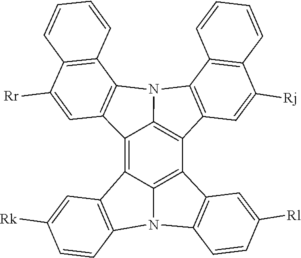 |
| Compound 184- (Rj)(Rk)(Rl)(Rr), wherein Compound 184- (R1)(R1)(R1)(R1) to Compound 184- (R468)(R468)(R468) (R232), have the structure | 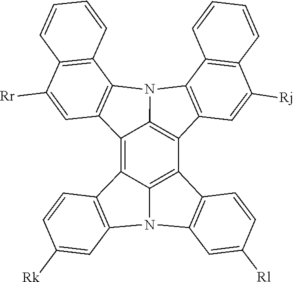 |
| Compound 185- (Rj)(Rk)(Rl)(Rr), wherein Compound 185- (R1)(R1)(R1)(R1) to Compound 185- (R468)(R468)(R468) (R232), have the structure | 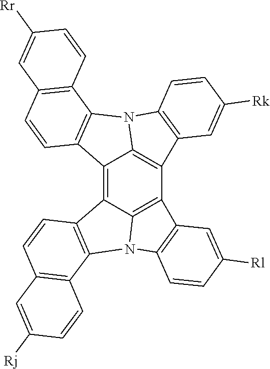 |
| Compound 186- (Rj)(Rk)(Rl)(Rr), wherein Compound 186- (R1)(R1)(R1)(R1) to Compound 186- (R468)(R468)(R468) (R232), have the structure | 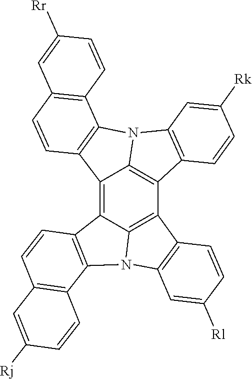 |
| Compound 187- (Rj)(Rk)(Rl)(Rr), wherein Compound 187- (R1)(R1)(R1)(R1) to Compound 187- (R468)(R468)(R468) (R232), have the structure | 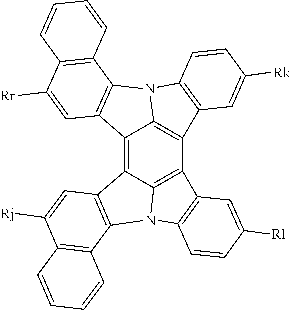 |
| Compound 188- (Rj)(Rk)(Rl)(Rr), wherein Compound 188- (R1)(R1)(R1)(R1) to Compound 188- (R468)(R468)(R468) (R232), have the structure | 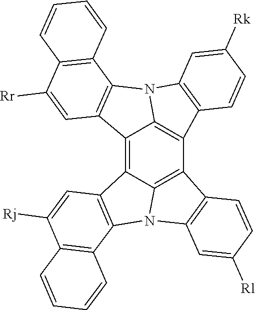 |
| Compound 189- (Rj)(Rk)(Rl)(Rr), wherein Compound 189- (R1)(R1)(R1)(R1) to Compound 189- (R468)(R468)(R468) (R232), have the structure | 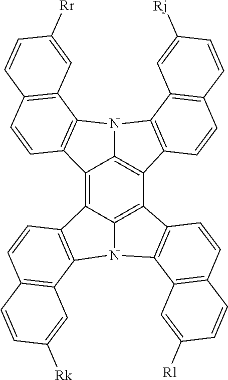 |
| Compound 190- (Rj)(Rk)(Rl)(Rr), wherein Compound 190- (R1)(R1)(R1)(R1) to Compound 190- (R468)(R468)(R468) (R232), have the structure | 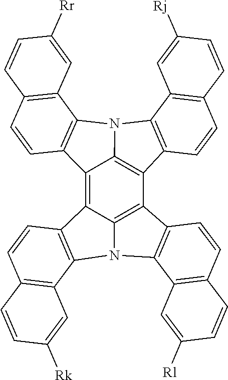 |
| Compound 191- (Rj)(Rk)(Rl)(Rr), wherein Compound 191- (R1)(R1)(R1)(R1) to Compound 191- (R468)(R468)(R468) (R232), have the structure | 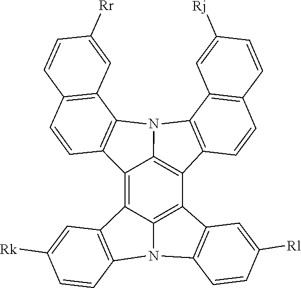 |
| Compound 192- (Rj)(Rk)(Rl)(Rr), wherein Compound 192- (R1)(R1)(R1)(R1) to Compound 192- (R468)(R468)(R468) (R232), have the structure | 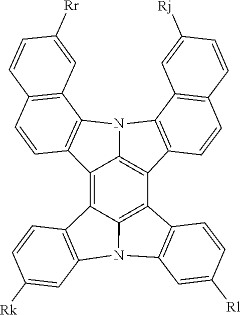 |
| Compound 193- (Rj)(Rk)(Rl)(Rr), wherein Compound 193- (R1)(R1)(R1)(R1) to Compound 193- (R468)(R468)(R468) (R232), have the structure | 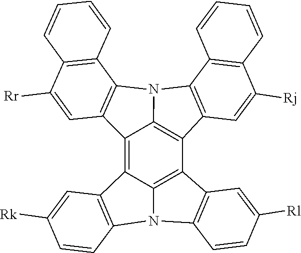 |
| Compound 194- (Rj)(Rk)(Rl)(Rr), wherein Compound 194- (R1)(R1)(R1)(R1) to Compound 194- (R468)(R468)(R468) (R232), have the structure | 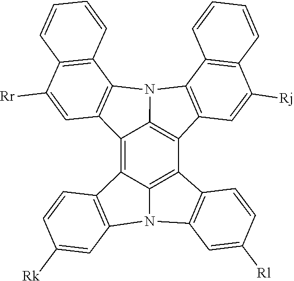 |
| Compound 195- (Rj)(Rk)(Rl)(Rr), wherein Compound 195- (R1)(R1)(R1)(R1) to Compound 195- (R468)(R468)(R468) (R232), have the structure | 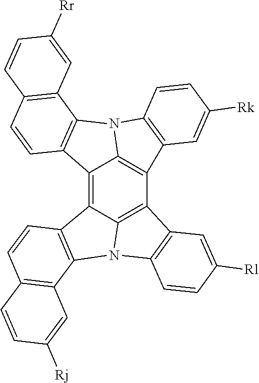 |
| Compound 196- (Rj)(Rk)(Rl)(Rr), wherein Compound 196- (R1)(R1)(R1)(R1) to Compound 196- (R468)(R468)(R468) (R232), have the structure | 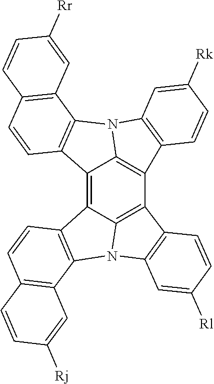 |
| Compound 197- (Rj)(Rk)(Rl)(Rr), wherein Compound 197- (R1)(R1)(R1)(R1) to Compound 197- (R468)(R468)(R468) (R232), have the structure | 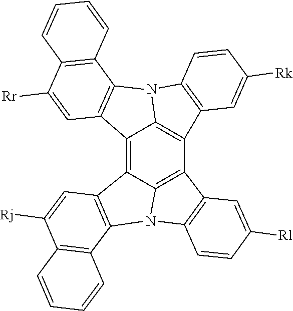 |
| Compound 198- (Rj)(Rk)(Rl)(Rr), wherein Compound 198- (R1)(R1)(R1)(R1) to Compound 198- (R468)(R468)(R468) (R232), have the structure | 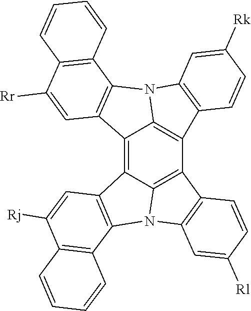 |
| Compound 199- (Rj)(Rk)(Rl)(Rr), wherein Compound 199- (R1)(R1)(R1)(R1) to Compound 199- (R468)(R468)(R468) (R232), have the structure | 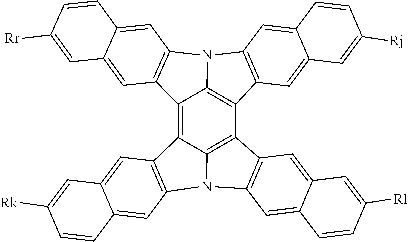 |
| Compound 200- (Rj)(Rk)(Rl)(Rr), wherein Compound 200- (R1)(R1)(R1)(R1) to Compound 200- (R468)(R468)(R468) (R232), have the structure | 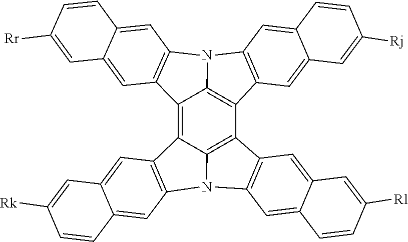 |
| Compound 201- (Rj)(Rk)(Rl)(Rr), wherein Compound 201- (R1)(R1)(R1)(R1) to Compound 201- (R468)(R468)(R468) (R232), have the structure | 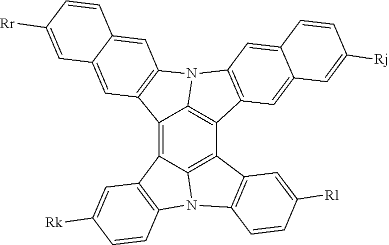 |
| Compound 202- (Rj)(Rk)(Rl)(Rr), wherein Compound 202- (R1)(R1)(R1)(R1) to Compound 202- (R468)(R468)(R468) (R232), have the structure | 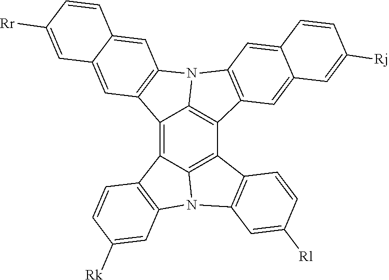 |
| Compound 203- (Rj)(Rk)(Rl)(Rr), wherein Compound 203- (R1)(R1)(R1)(R1) to Compound 203- (R468)(R468)(R468) (R232), have the structure | 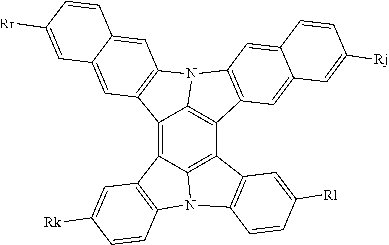 |
| Compound 204- (Rj)(Rk)(Rl)(Rr), wherein Compound 204- (R1)(R1)(R1)(R1) to Compound 204- (R468)(R468)(R468) (R232), have the structure | 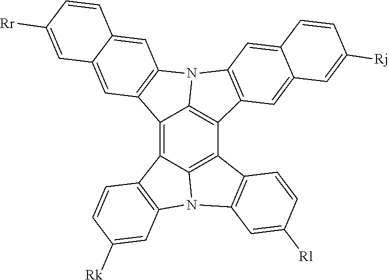 |
| Compound 205- (Rj)(Rk)(Rl)(Rr), wherein Compound 205- (R1)(R1)(R1)(R1) to Compound 205- (R468)(R468)(R468) (R232), have the structure | 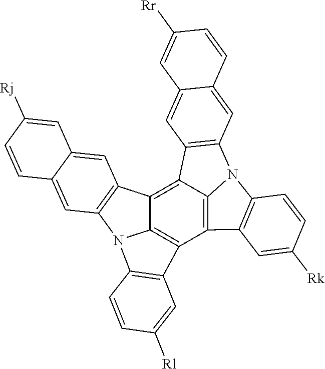 |
| Compound 206- (Rj)(Rk)(Rl)(Rr), wherein Compound 206- (R1)(R1)(R1)(R1) to Compound 206- (R468)(R468)(R468) (R232), have the structure | 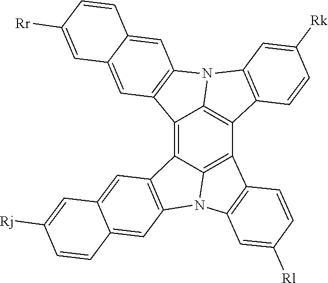 |
| Compound 207- (Rj)(Rk)(Rl)(Rr), wherein Compound 207- (R1)(R1)(R1)(R1) to Compound 207- (R468)(R468)(R468) (R232), have the structure | 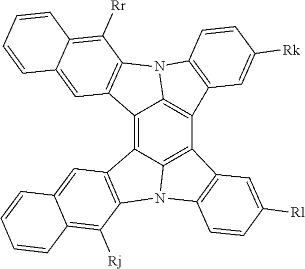 |
| Compound 208- (Rj)(Rk)(Rl)(Rr), wherein Compound 208- (R1)(R1)(R1)(R1) to Compound 208- (R468)(R468)(R468) (R232), have the structure | 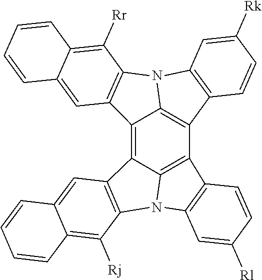 |
| Compound 209- (Rj)(Rk)(Rl)(Rr), wherein Compound 209- (R1)(R1)(R1)(R1) to Compound 209- (R468)(R468)(R468) (R232), have the structure | 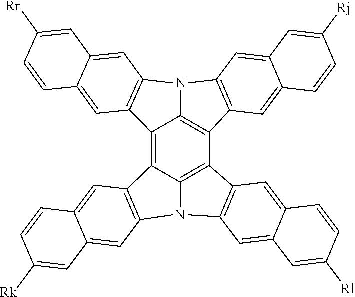 |
| Compound 210- (Rj)(Rk)(Rl)(Rr), wherein Compound 210- (R1)(R1)(R1)(R1) to Compound 210- (R468)(R468)(R468) (R232), have the structure | 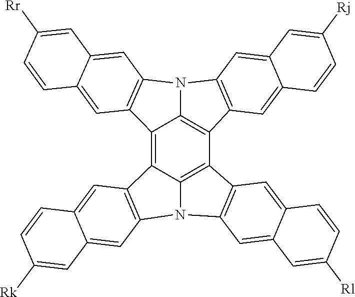 |
| Compound 211- (Rj)(Rk)(Rl)(Rr), wherein Compound 211- (R1)(R1)(R1)(R1) to Compound 211- (R468)(R468)(R468) (R232), have the structure | 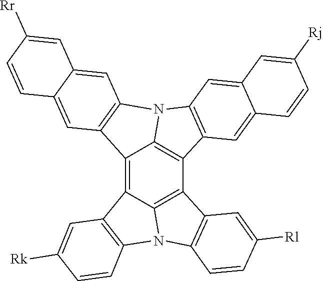 |
| Compound 212- (Rj)(Rk)(Rl)(Rr), wherein Compound 212- (R1)(R1)(R1)(R1) to Compound 212- (R468)(R468)(R468) (R232), have the structure | 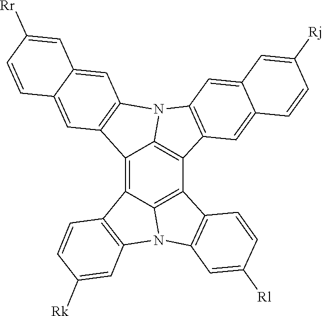 |
| Compound 213- (Rj)(Rk)(Rl)(Rr), wherein Compound 213- (R1)(R1)(R1)(R1) to Compound 213- (R468)(R468)(R468) (R232), have the structure | 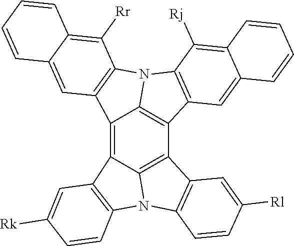 |
| Compound 214- (Rj)(Rk)(Rl)(Rr), wherein Compound 214- (R1)(R1)(R1)(R1) to Compound 214- (R468)(R468)(R468) (R232), have the structure | 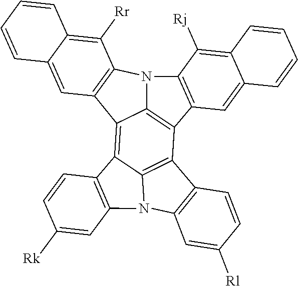 |
| Compound 215- (Rj)(Rk)(Rl)(Rr), wherein Compound 215- (R1)(R1)(R1)(R1) to Compound 215- (R468)(R468)(R468) (R232), have the structure | 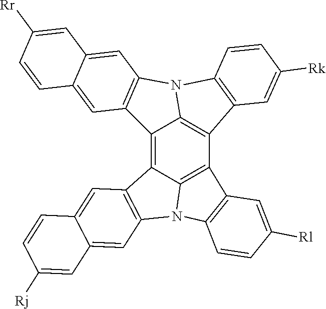 |
| Compound 216- (Rj)(Rk)(Rl)(Rr), wherein Compound 216- (R1)(R1)(R1)(R1) to Compound 216- (R468)(R468)(R468) (R232), have the structure | 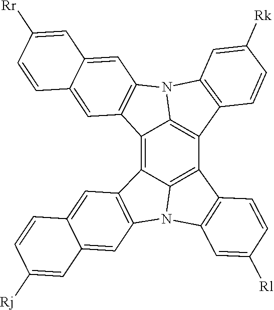 |
| Compound 217- (Rj)(Rk)(Rl)(Rr), wherein Compound 217- (R1)(R1)(R1)(R1) to Compound 217- (R468)(R468)(R468) (R232), have the structure | 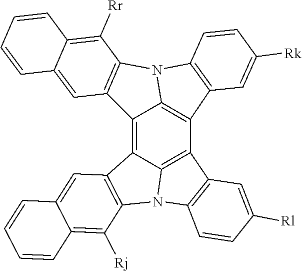 |
| Compound 218- (Rj)(Rk)(Rl)(Rr), wherein Compound 218- (R1)(R1)(R1)(R1) to Compound 218- (R468)(R468)(R468) (R232), have the structure | 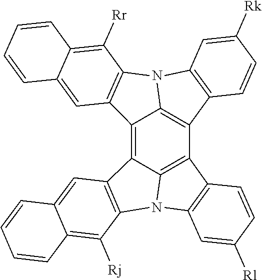 |
| Compound 219- (Rj)(Rk)(Rl)(Rr), wherein Compound 219- (R1)(R1)(R1)(R1) to Compound 219- (R468)(R468)(R468) (R232), have the structure | 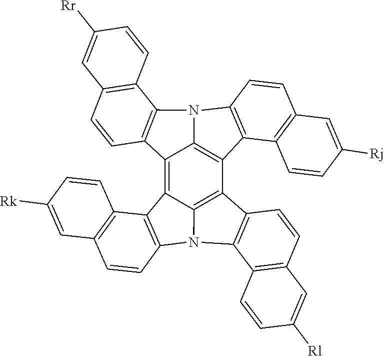 |
| Compound 220- (Rj)(Rk)(Rl)(Rr), wherein Compound 220- (R1)(R1)(R1)(R1) to Compound 220- (R468)(R468)(R468) (R232), have the structure | 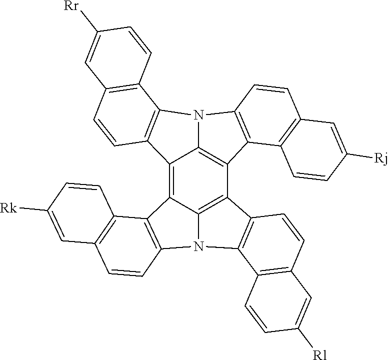 |
| Compound 221- (Rj)(Rk)(Rl)(Rr), wherein Compound 221- (R1)(R1)(R1)(R1) to Compound 221- (R468)(R468)(R468) (R232), have the structure | 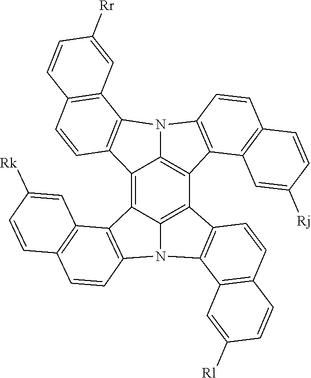 |
| Compound 222- (Rj)(Rk)(Rl)(Rr), wherein Compound 222- (R1)(R1)(R1)(R1) to Compound 222- (R468)(R468)(R468) (R232), have the structure | 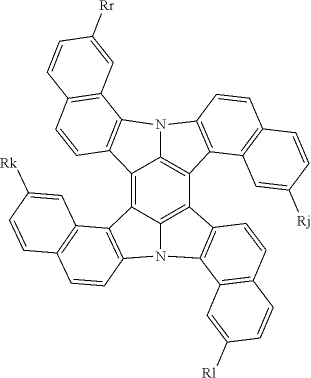 |
| Compound 223- (Rj)(Rk)(Rl)(Rs), wherein Compound 223- (R1)(R1)(R1)(R1) to Compound 223- (R468)(R468)(R468) (R209), have the structure | 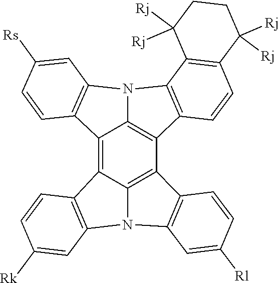 |
| Compound 224- (Rj)(Rk)(Rl)(Rs), wherein Compound 224- (R1)(R1)(R1)(R1) to Compound 224- (R468)(R468)(R468) (R209), have the structure | 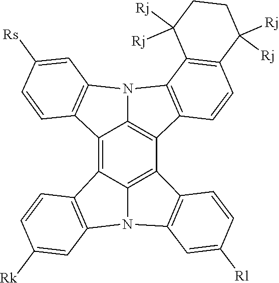 |
| Compound 225- (Rj)(Rk)(Rl)(Rs), wherein Compound 225- (R1)(R1)(R1)(R1) to Compound 225- (R468)(R468)(R468) (R209), have the structure | 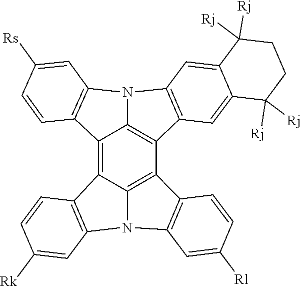 |
| Compound 226- (Rj)(Rk)(Rl)(Rs), wherein Compound 226- (R1)(R1)(R1)(R1) to Compound 226- (R468)(R468)(R468) (R209), have the structure | 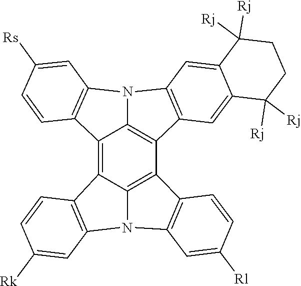 |
| Compound 227- (Rj)(Rk)(Rl)(Rs), wherein Compound 227- (R1)(R1)(R1)(R1) to Compound 227- (R468)(R468)(R468) (R209), have the structure | 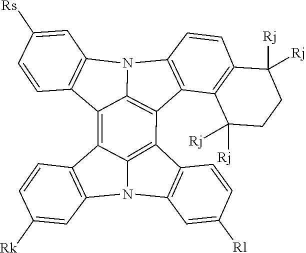 |
| Compound 228- (Rj)(Rk)(Rl)(Rs), wherein Compound 228- (R1)(R1)(R1)(R1) to Compound 228- (R468)(R468)(R468) (R209), have the structure | 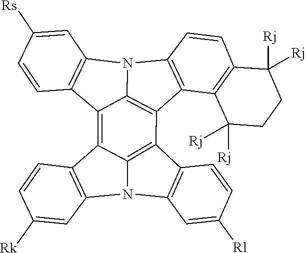 |
| Compound 229- (Rj)(Rk)(Rl)(Rs), wherein Compound 229- (R1)(R1)(R1)(R1) to Compound 229- (R468)(R468)(R468) (R209), have the structure | 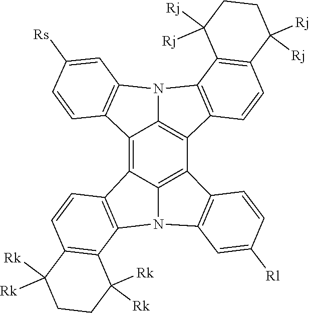 |
| Compound 230- (Rj)(Rk)(Rl)(Rs), wherein Compound 230- (R1)(R1)(R1)(R1) to Compound 230- (R468)(R468)(R468) (R209), have the structure | 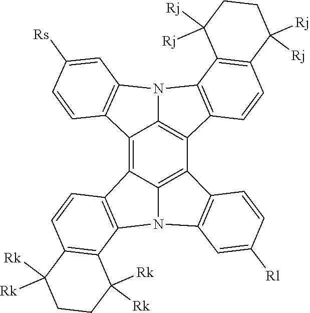 |
| Compound 231- (Rj)(Rk)(Rl)(Rs), wherein Compound 231- (R1)(R1)(R1)(R1) to Compound 231- (R468)(R468)(R468) (R209), have the structure | 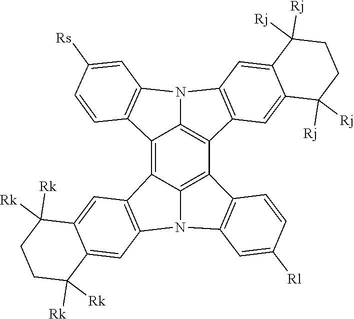 |
| Compound 232- (Rj)(Rk)(Rl)(Rs), wherein Compound 232- (R1)(R1)(R1)(R1) to Compound 232- (R468)(R468)(R468) (R209), have the structure | 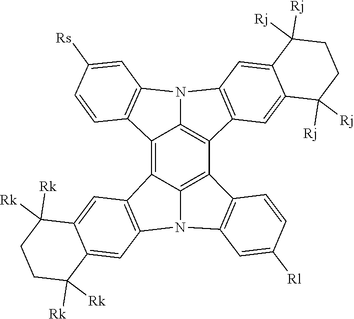 |
| Compound 233- (Rj)(Rk)(Rl)(Rs), wherein Compound 233- (R1)(R1)(R1)(R1) to Compound 233- (R468)(R468)(R468) (R209), have the structure | 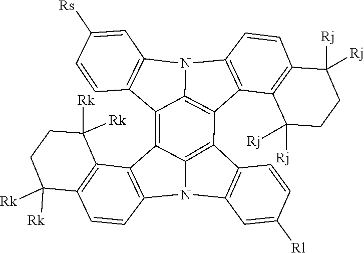 |
| Compound 234- (Rj)(Rk)(Rl)(Rs), wherein Compound 234- (R1)(R1)(R1)(R1) to Compound 234- (R468)(R468)(R468) (R209), have the structure | 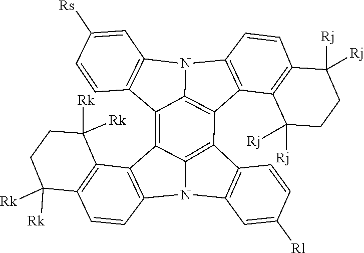 |
| Compound 235- (Ri)(Rj)(Rk)(Rl), wherein Compound 235- (R1)(R1)(R1)(R1) to Compound 235- (R468)(R468) (R468)(R468), have the structure | 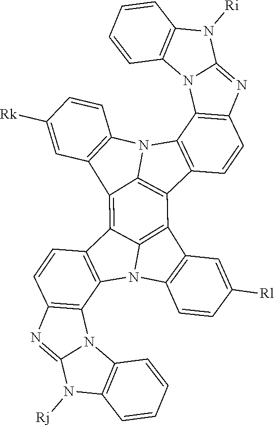 |
| Compound 236- (Ri)(Rj)(Rk)(Rl), wherein Compound 236- (R1)(R1)(R1)(R1) to Compound 236- (R468)(R468) (R468)(R468), have the structure | 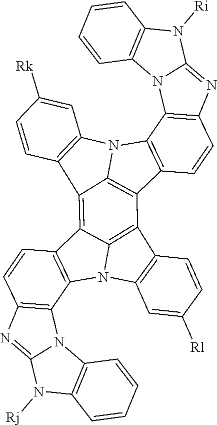 |
| Compound 237- (Ri)(Rj)(Rk)(Rl), wherein Compound 237- (R1)(R1)(R1)(R1) to Compound 237- (R468)(R468) (R468)(R468), have the structure | 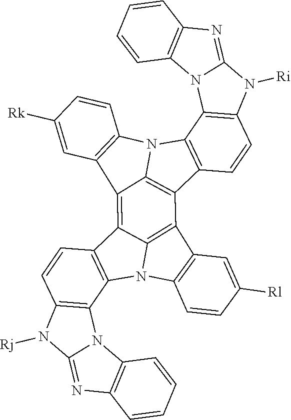 |
| Compound 238- (Ri)(Rj)(Rk)(Rl), wherein Compound 238- (R1)(R1)(R1)(R1) to Compound 238- (R468)(R468) (R468)(R468), have the structure | 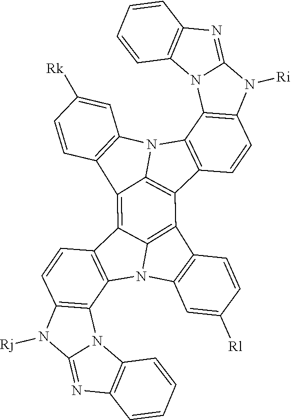 |
| Compound 239- (Ri)(Rj)(Rk)(Rl), wherein Compound 239- (R1)(R1)(R1)(R1) to Compound 239- (R468)(R468) (R468)(R468), have the structure | 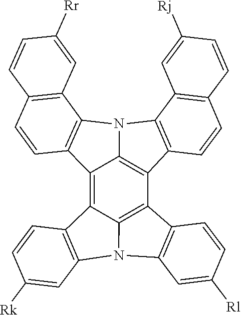 |
| Compound 240- (Ri)(Rj)(Rk)(Rl), wherein Compound 240- (R1)(R1)(R1)(R1) to Compound 240- (R468)(R468) (R468)(R468), have the structure | 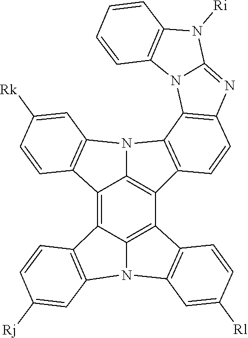 |
| Compound 241- (Ri)(Rj)(Rk)(Rl), wherein Compound 241- (R1)(R1)(R1)(R1) to Compound 241- (R468)(R468) (R468)(R468), have the structure | 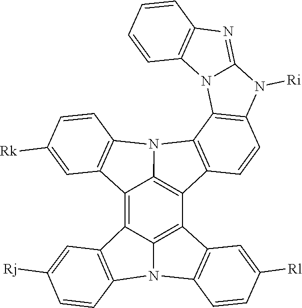 |
| Compound 242- (Ri)(Rj)(Rk)(Rl), wherein Compound 242- (R1)(R1)(R1)(R1) to Compound 242- (R468)(R468) (R468)(R468), have the structure | 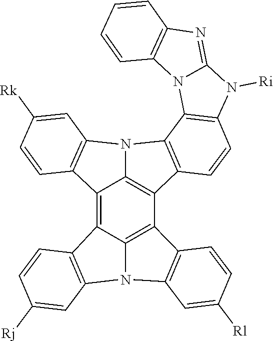 |
| Compound 243- (Ri)(Rj)(Rk)(Rl), wherein Compound 243- (R1)(R1)(R1)(R1) to Compound 243- (R468)(R468) (R468)(R468), have the structure | 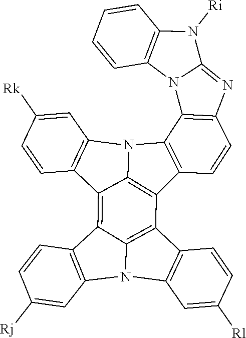 |
| Compound 244- (Ri)(Rj)(Rk)(Rl), wherein Compound 244- (R1)(R1)(R1)(R1) to Compound 244- (R468)(R468) (R468)(R468), have the structure | 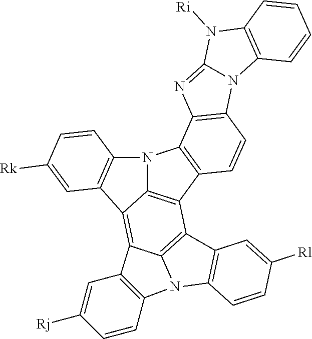 |
| Compound 245- (Ri)(Rj)(Rk)(Rl), wherein Compound 245- (R1)(R1)(R1)(R1) to Compound 245- (R468)(R468) (R468)(R468), have the structure | 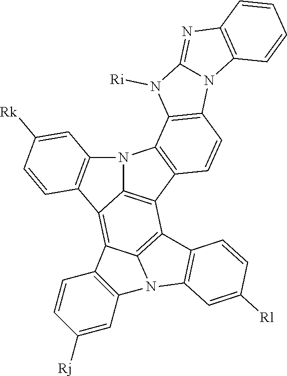 |
| Compound 246- (Ri)(Rj)(Rk)(Rl), wherein Compound 246- (R1)(R1)(R1)(R1) to Compound 246- (R468)(R468) (R468)(R468), have the structure | 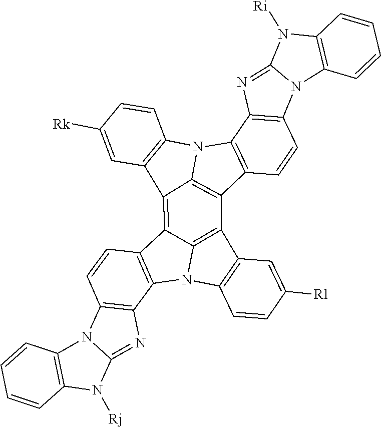 |
| Compound 247- (Ri)(Rj)(Rk)(Rl), wherein Compound 247- (R1)(R1)(R1)(R1) to Compound 247- (R468)(R468) (R468)(R468), have the structure | 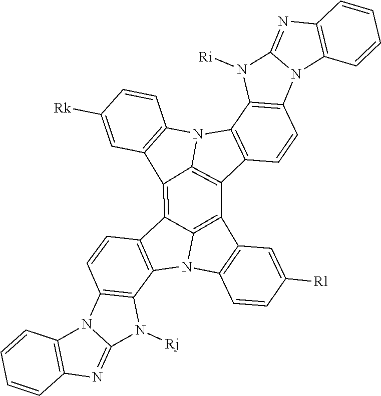 |
| Compound 248- (Ri)(Rj)(Rk)(Rl), wherein Compound 248- (R1)(R1)(R1)(R1) to Compound 248- (R468)(R468) (R468)(R468), have the structure | 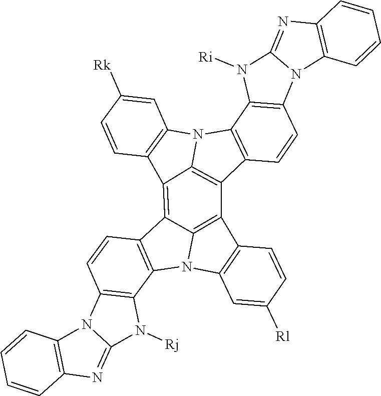 |
| Compound 249- (Ri)(Rj)(Rk)(Rl), wherein Compound 249- (R1)(R1)(R1)(R1) to Compound 249- (R468)(R468) (R468)(R468), have the structure | 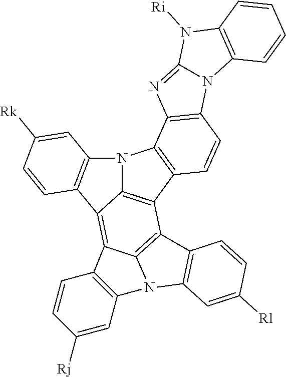 |
| Compound 250- (Ri)(Rj)(Rk)(Rl), wherein Compound 250- (R1)(R1)(R1)(R1) to Compound 250- (R468)(R468) (R468)(R468), have the structure | 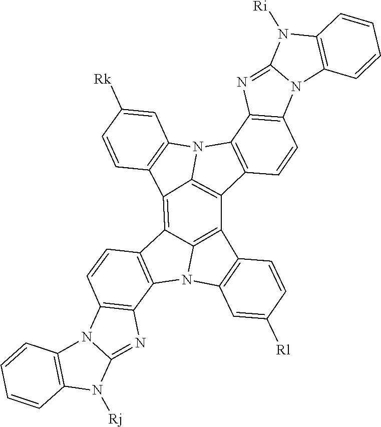 |
| Compound 251- (Ri)(Rj)(Rk)(Rl), wherein Compound 251- (R1)(R1)(R1)(R1) to Compound 251- (R468)(R468) (R468)(R468), have the structure | 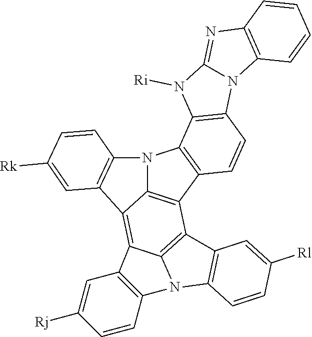 |
| Compound 252- (Ri)(Rj)(Rk)(Rl), wherein Compound 252- (R1)(R1)(R1)(R1) to Compound 252- (R468)(R468) (R468)(R468), have the structure | 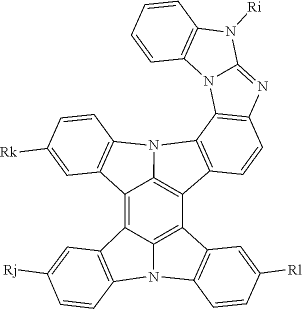 |
| Compound 253- (Ri)(Rj)(Rk)(Rl), wherein Compound 253- (R1)(R1)(R1)(R1) to Compound 253- (R468)(R468) (R468)(R468), have the structure | 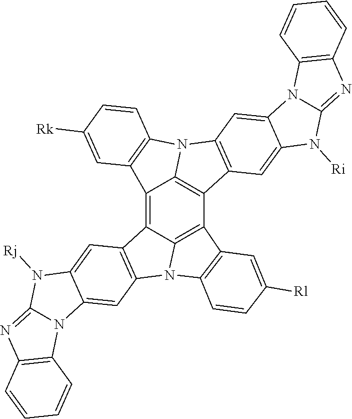 |
| Compound 254- (Ri)(Rj)(Rk)(Rl), wherein Compound 254- (R1)(R1)(R1)(R1) to Compound 254- (R468)(R468) (R468)(R468), have the structure | 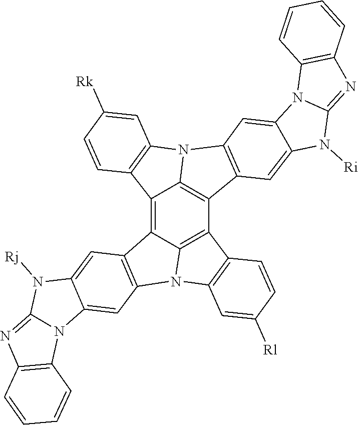 |
| Compound 255- (Ri)(Rj)(Rk)(Rl), wherein Compound 255- (R1)(R1)(R1)(R1) to Compound 255- (R468)(R468) (R468)(R468), have the structure | 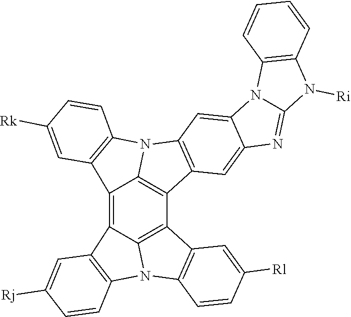 |
| Compound 256- (Ri)(Rj)(Rk)(Rl), wherein Compound 256- (R1)(R1)(R1)(R1) to Compound 256- (R468)(R468) (R468)(R468), have the structure | 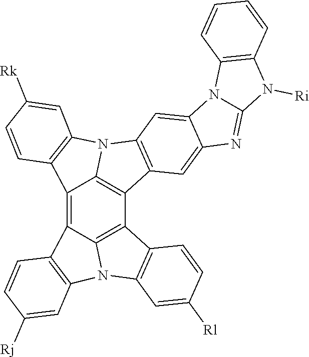 |
| Compound 257- (Ri)(Rj)(Rk)(Rl), wherein Compound 257- (R1)(R1)(R1)(R1) to Compound 257- (R468)(R468) (R468)(R468), have the structure | 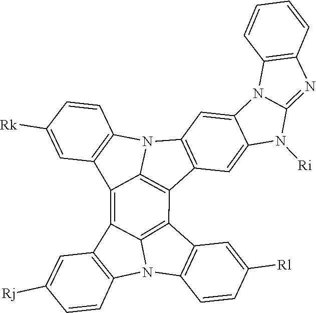 |
| Compound 258- (Ri)(Rj)(Rk)(Rl), wherein Compound 258- (R1)(R1)(R1)(R1) to Compound 258- (R468)(R468) (R468)(R468), have the structure | 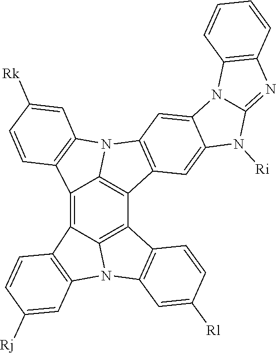 |
| Compound 259- (Ri)(Rj)(Rk)(Rl), wherein Compound 259- (R1)(R1)(R1)(R1) to Compound 259- (R468)(R468) (R468)(R468), have the structure | 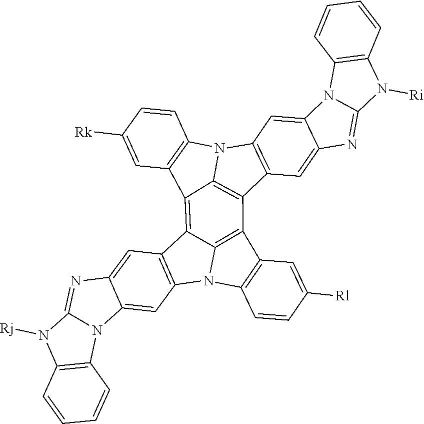 |
| Compound 260- (Ri)(Rj)(Rk)(Rl), wherein Compound 260- (R1)(R1)(R1)(R1) to Compound 260- (R468)(R468) (R468)(R468), have the structure | 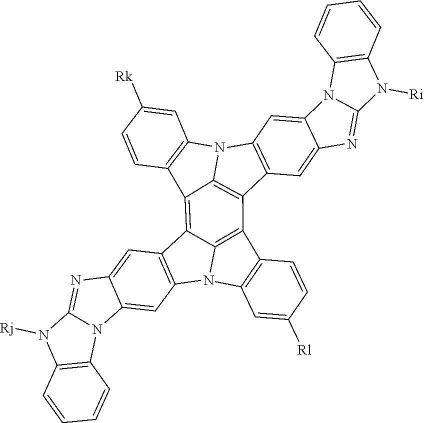 |
| Compound 261- (Ri)(Rj)(Rk)(Rl), wherein Compound 261- (R1)(R1)(R1)(R1) to Compound 261- (R468)(R468) (R468)(R468), have the structure | 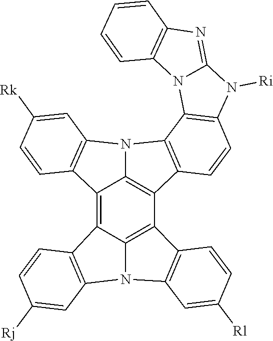 |
| Compound 262- (Ri)(Rj)(Rk)(Rl), wherein Compound 262- (R1)(R1)(R1)(R1) to Compound 262- (R468)(R468) (R468)(R468), have the structure | 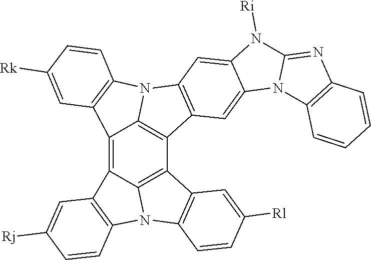 |
| Compound 263- (Ri)(Rj)(Rk)(Rl), wherein Compound 263- (R1)(R1)(R1)(R1) to Compound 263- (R468)(R468) (R468)(R468), have the structure | 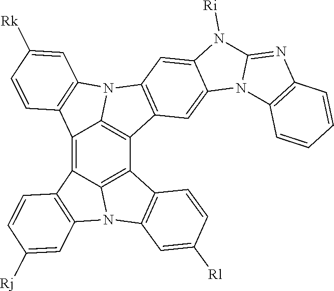 |
| Compound 264- (Ri)(Rj)(Rk)(Rl), wherein Compound 264- (R1)(R1)(R1)(R1) to Compound 264- (R468)(R468) (R468)(R468), have the structure | 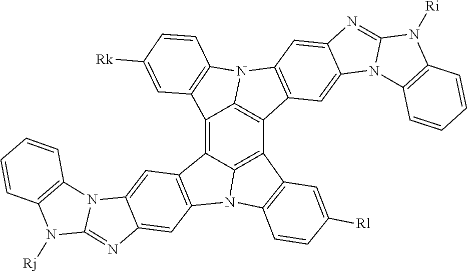 |
| Compound 265- (Ri)(Rj)(Rk)(Rl), wherein Compound 265- (R1)(R1)(R1)(R1) to Compound 265- (R468)(R468) (R468)(R468), have the structure | 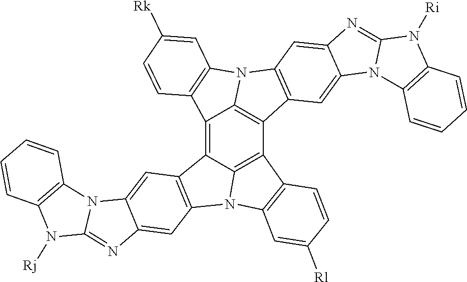 |
| Compound 266- (Ri)(Rj)(Rk)(Rl), wherein Compound 266- (R1)(R1)(R1)(R1) to Compound 266- (R468)(R468) (R468)(R468), have the structure | 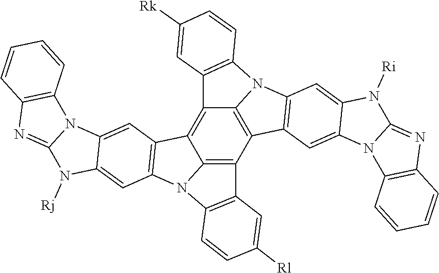 |
| Compound 267- (Ri)(Rj)(Rk)(Rl), wherein Compound 267- (R1)(R1)(R1)(R1) to Compound 267- (R468)(R468) (R468)(R468), have the structure | 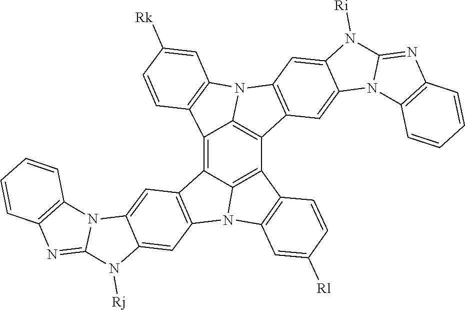 |
| Compound 268- (Ri)(Rj)(Rk)(Rl), wherein Compound 268- (R1)(R1)(R1)(R1) to Compound 268- (R468)(R468) (R468)(R468), have the structure | 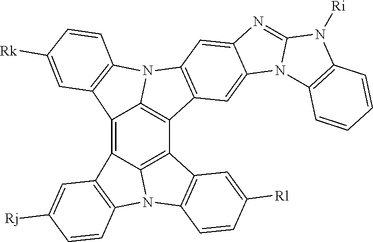 |
| Compound 269- (Ri)(Rj)(Rk)(Rl), wherein Compound 269- (R1)(R1)(R1)(R1) to Compound 269- (R468)(R468) (R468)(R468), have the structure | 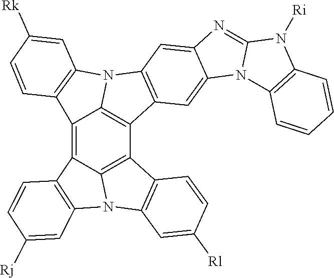 |
| Compound 270- (Ri)(Rj)(Rk)(Rl), wherein Compound 270- (R1)(R1)(R1)(R1) to Compound 270- (R468)(R468) (R468)(R468), have the structure | 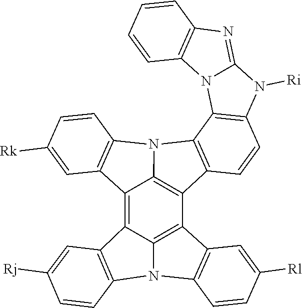 |
| Compound 271- (Ri)(Rj)(Rk)(Rl), wherein Compound 271- (R1)(R1)(R1)(R1) to Compound 271- (R468)(R468) (R468)(R468), have the structure | 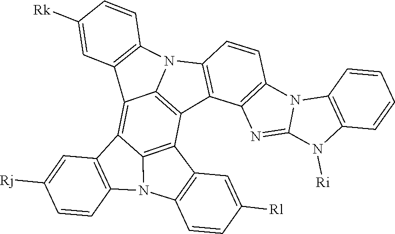 |
| Compound 272- (Ri)(Rj)(Rk)(Rl), wherein Compound 272- (R1)(R1)(R1)(R1) to Compound 272- (R468)(R468) (R468)(R468), have the structure | 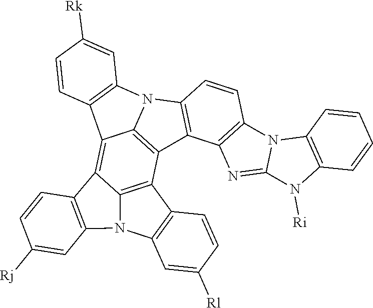 |
| Compound 273- (Ri)(Rj)(Rk)(Rl), wherein Compound 273- (R1)(R1)(R1)(R1) to Compound 273- (R468)(R468) (R468)(R468), have the structure | 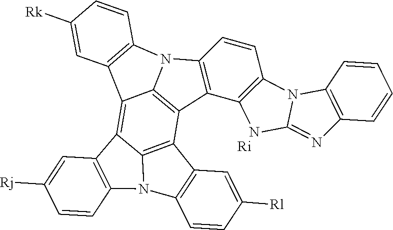 |
| Compound 274- (Ri)(Rj)(Rk)(Rl), wherein Compound 274- (R1)(R1)(R1)(R1) to Compound 274- (R468)(R468) (R468)(R468), have the structure | 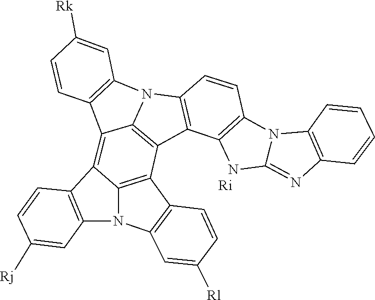 |
| Compound 275- (Ri)(Rj)(Rk)(Rl), wherein Compound 275- (R1)(R1)(R1)(R1) to Compound 275- (R468)(R468) (R468)(R468), have the structure | 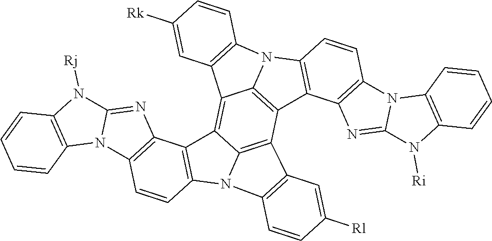 |
| Compound 276- (Ri)(Rj)(Rk)(Rl), wherein Compound 276- (R1)(R1)(R1)(R1) to Compound 276- (R468)(R468) (R468)(R468), have the structure | 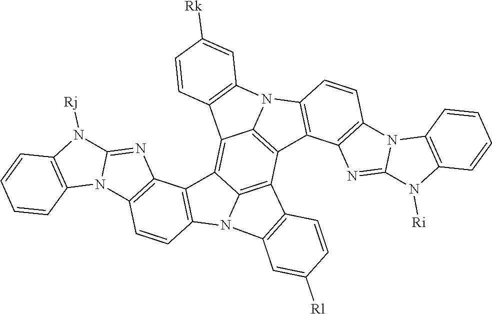 |
| Compound 277- (Ri)(Rj)(Rk)(Rl), wherein Compound 277- (R1)(R1)(R1)(R1) to Compound 277- (R468)(R468) (R468)(R468), have the structure | 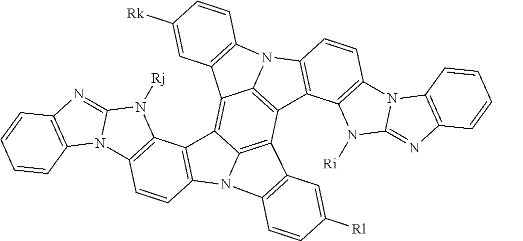 |
| Compound 278- (Ri)(Rj)(Rk)(Rl), wherein Compound 278- (R1)(R1)(R1)(R1) to Compound 278- (R468)(R468) (R468)(R468), have the structure | 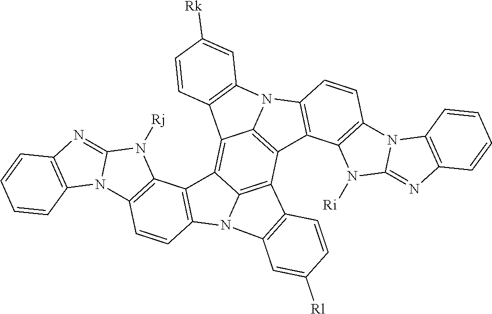 |
| Compound 279- (Ri)(Rj)(Rk)(Rl), wherein Compound 279- (R1)(R1)(R1)(R1) to Compound 279- (R468)(R468) (R468)(R468), have the structure | 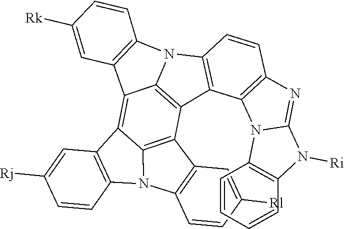 |
| Compound 280- (Ri)(Rj)(Rk)(Rl), wherein Compound 280- (R1)(R1)(R1)(R1) to Compound 280- (R468)(R468) (R468)(R468), have the structure | 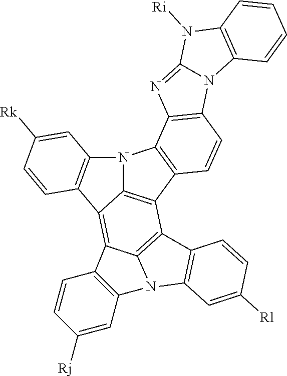 |
| Compound 281- (Ri)(Rj)(Rk)(Rl), wherein Compound 281- (R1)(R1)(R1)(R1) to Compound 281- (R468)(R468) (R468)(R468), have the structure | 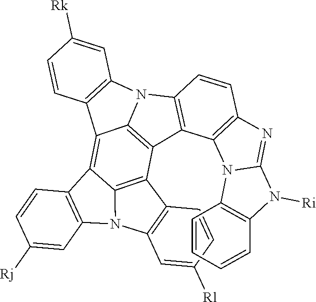 |
| Compound 282- (Ri)(Rj)(Rk)(Rl), wherein Compound 282- (R1)(R1)(R1)(R1) to Compound 282- (R468)(R468) (R468)(R468), have the structure | 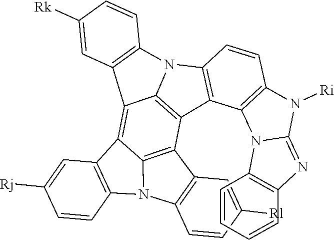 |
| Compound 283- (Ri)(Rj)(Rk)(Rl), wherein Compound 283- (R1)(R1)(R1)(R1) to Compound 283- (R468)(R468) (R468)(R468), have the structure | 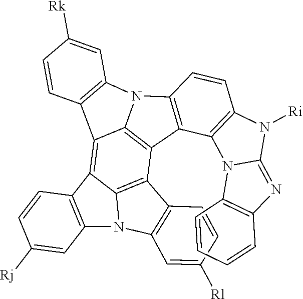 |
| Compound 284- (Ri)(Rj)(Rk)(Rl), wherein Compound 284- (R1)(R1)(R1)(R1) to Compound 284- (R468)(R468) (R468)(R468), have the structure | 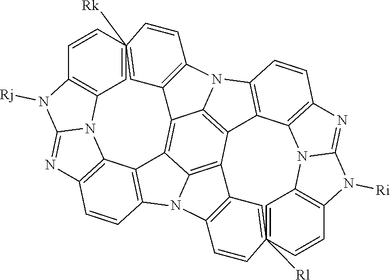 |
| Compound 285- (Ri)(Rj)(Rk)(Rl), wherein Compound 285- (R1)(R1)(R1)(R1) to Compound 285- (R468)(R468) (R468)(R468), have the structure | 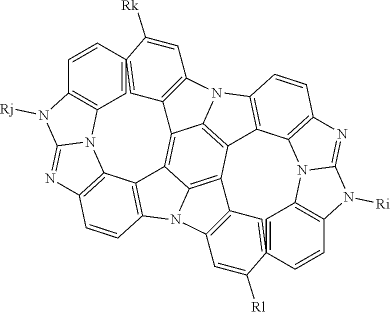 |
| Compound 286- (Ri)(Rj)(Rk)(Rl), wherein Compound 286- (R1)(R1)(R1)(R1) to Compound 286- (R468)(R468) (R468)(R468), have the structure | 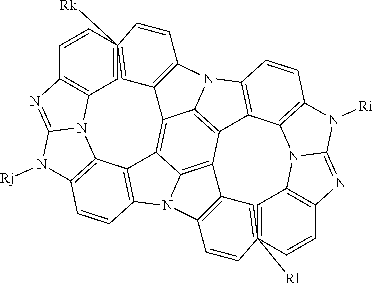 |
| Compound 287- (Ri)(Rj)(Rk)(Rl), wherein Compound 287- (R1)(R1)(R1)(R1) to Compound 287- (R468)(R468) (R468)(R468), have the structure | 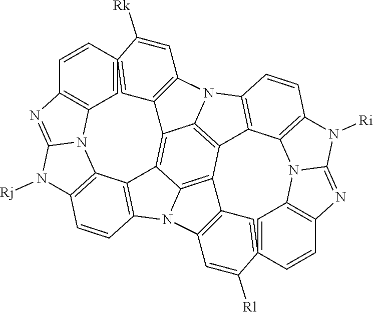 |
| Compound 288- (Ri)(Rj)(Rk)(Rl), wherein Compound 288- (R1)(R1)(R1)(R1) to Compound 288- (R468)(R468) (R468)(R468), have the structure | 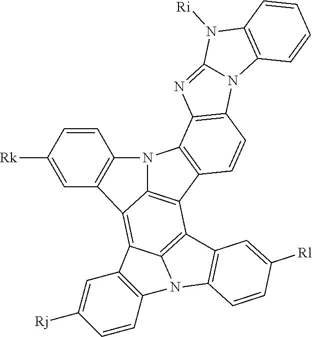 |
| Compound 289- (Ri)(Rj)(Rk)(Rl), wherein Compound 289- (R1)(R1)(R1)(R1) to Compound 289- (R468)(R468) (R468)(R468), have the structure | 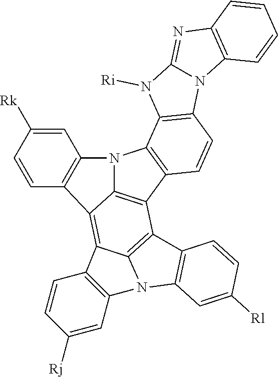 |
| Compound 290- (Ri)(Rj)(Rk)(Rl), wherein Compound 290- (R1)(R1)(R1)(R1) to Compound 290- (R468)(R468) (R468)(R468), have the structure | 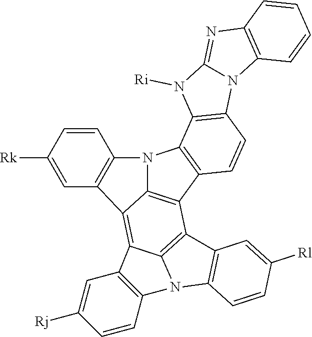 |
| Compound 291- (Ri)(Rj)(Rk)(Rl), wherein Compound 291- (R1)(R1)(R1)(R1) to Compound 291- (R468)(R468) (R468)(R468), have the structure | 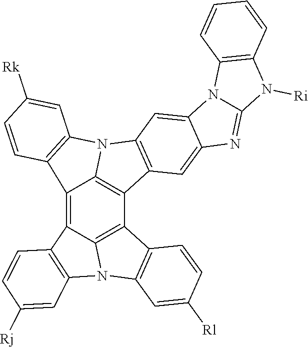 |
| Compound 292- (Ri)(Rj)(Rk)(Rl), wherein Compound 292- (R1)(R1)(R1)(R1) to Compound 292- (R468)(R468) (R468)(R468), have the structure | 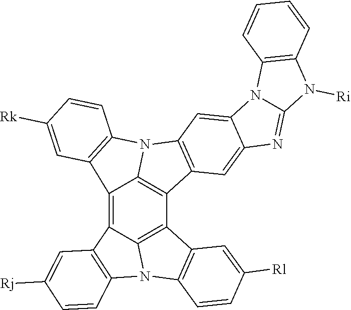 |
| Compound 293- (Ri)(Rj)(Rk)(Rl), wherein Compound 293- (R1)(R1)(R1)(R1) to Compound 293- (R468)(R468) (R468)(R468), have the structure | 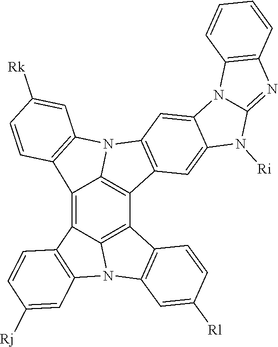 |
| Compound 294- (Ri)(Rj)(Rk)(Rl), wherein Compound 294- (R1)(R1)(R1)(R1) to Compound 294- (R468)(R468) (R468)(R468), have the structure | 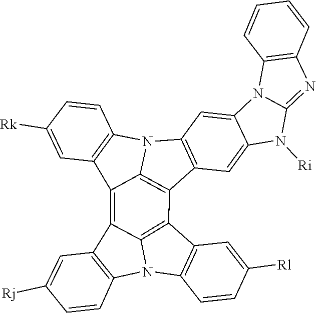 |
| Compound 295- (Ri)(Rj)(Rk)(Rl), wherein Compound 295- (R1)(R1)(R1)(R1) to Compound 295- (R468)(R468) (R468)(R468), have the structure | 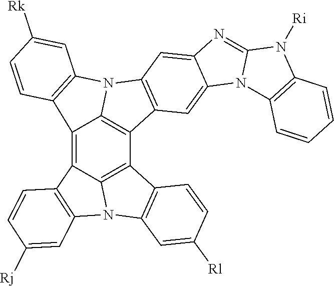 |
| Compound 296- (Ri)(Rj)(Rk)(Rl), wherein Compound 296- (R1)(R1)(R1)(R1) to Compound 296- (R468)(R468) (R468)(R468), have the structure | 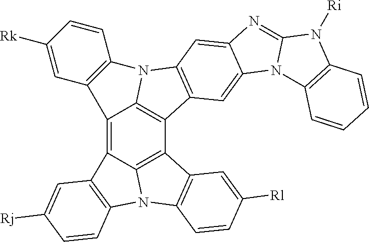 |
| Compound 297- (Ri)(Rj)(Rk)(Rl), wherein Compound 297- (R1)(R1)(R1)(R1) to Compound 297- (R468)(R468) (R468)(R468), have the structure | 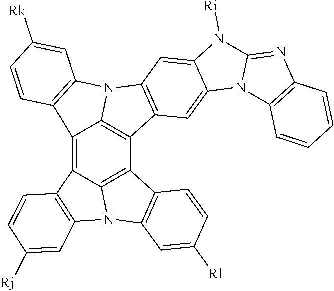 |
| Compound 298- (Ri)(Rj)(Rk)(Rl), wherein Compound 298- (R1)(R1)(R1)(R1) to Compound 298- (R468)(R468) (R468)(R468), have the structure | 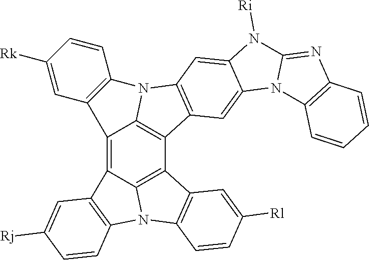 |
| Compound 299- (Ri)(Rj)(Rk)(Rl), wherein Compound 299- (R1)(R1)(R1)(R1) to Compound 299- (R468)(R468) (R468)(R468), have the structure | 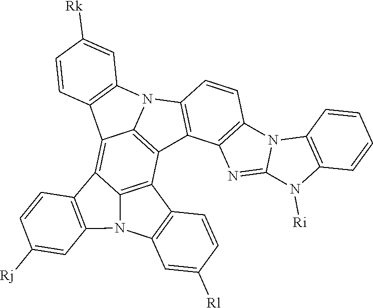 |
| Compound 300- (Ri)(Rj)(Rk)(Rl), wherein Compound 300- (R1)(R1)(R1)(R1) to Compound 300- (R468)(R468) (R468)(R468), have the structure | 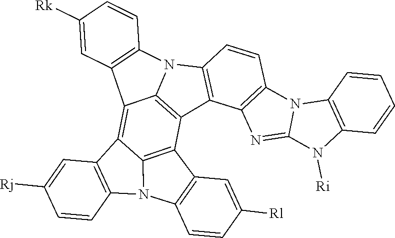 |
| Compound 301- (Ri)(Rj)(Rk)(Rl), wherein Compound 301- (R1)(R1)(R1)(R1) to Compound 301- (R468)(R468) (R468)(R468), have the structure | 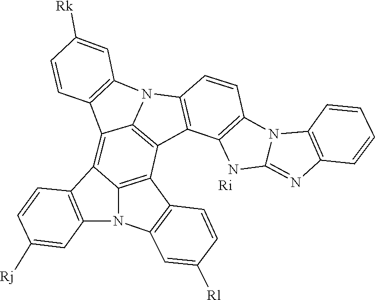 |
| Compound 302- (Ri)(Rj)(Rk)(Rl), wherein Compound 302- (R1)(R1)(R1)(R1) to Compound 302- (R468)(R468) (R468)(R468), have the structure | 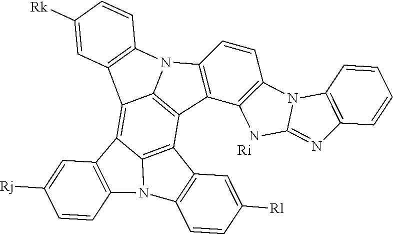 |
| Compound 303- (Ri)(Rj)(Rk)(Rl), wherein Compound 303- (R1)(R1)(R1)(R1) to Compound 303- (R468)(R468) (R468)(R468), have the structure | 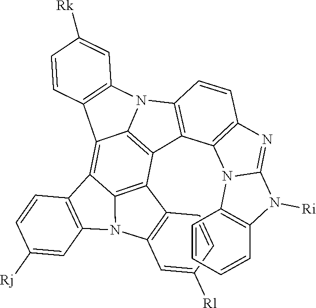 |
| Compound 304- (Ri)(Rj)(Rk)(Rl), wherein Compound 304- (R1)(R1)(R1)(R1) to Compound 304- (R468)(R468) (R468)(R468), have the structure | 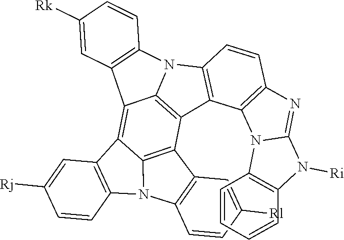 |
| Compound 305- (Ri)(Rj)(Rk)(Rl), wherein Compound 305- (R1)(R1)(R1)(R1) to Compound 305- (R468)(R468) (R468)(R468), have the structure | 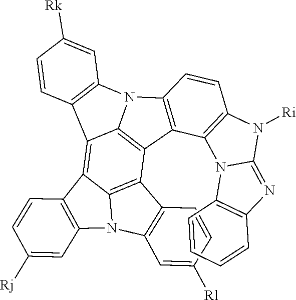 |
| Compound 306- (Ri)(Rj)(Rk)(Rl), wherein Compound 306- (R1)(R1)(R1)(R1) to Compound 306- (R468)(R468) (R468)(R468), have the structure | 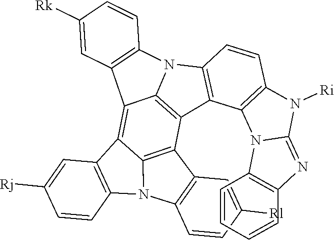 |
| Compound 307- (Ri)(Rj)(Rk)(Rl), wherein Compound 307- (R1)(R1)(R1)(R1) to Compound 307- (R468)(R468) (R468)(R468), have the structure | 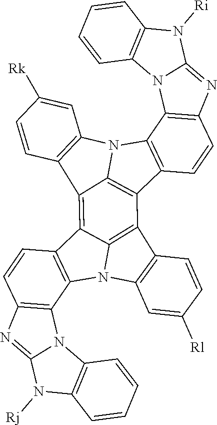 |
| Compound 308- (Ri)(Rj)(Rk)(Rl), wherein Compound 308- (R1)(R1)(R1)(R1) to Compound 308- (R468)(R468) (R468)(R468), have the structure | 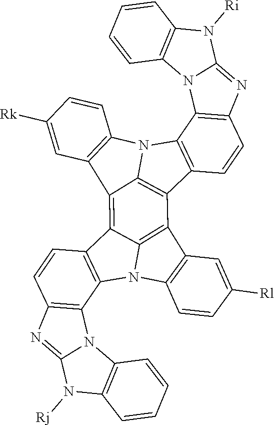 |
| Compound 309- (Ri)(Rj)(Rk)(Rl), wherein Compound 309- (R1)(R1)(R1)(R1) to Compound 309- (R468)(R468) (R468)(R468), have the structure | 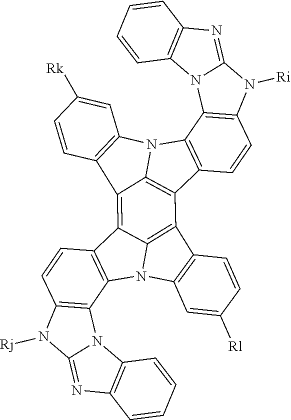 |
| Compound 310- (Ri)(Rj)(Rk)(Rl), wherein Compound 310- (R1)(R1)(R1)(R1) to Compound 310- (R468)(R468) (R468)(R468), have the structure | 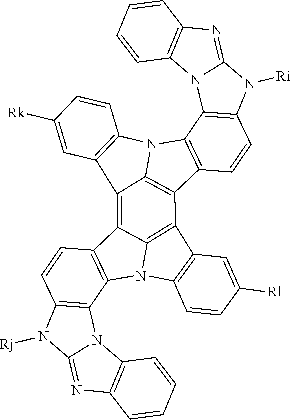 |
| Compound 311- (Ri)(Rj)(Rk)(Rl), wherein Compound 311- (R1)(R1)(R1)(R1) to Compound 311- (R468)(R468) (R468)(R468), have the structure | 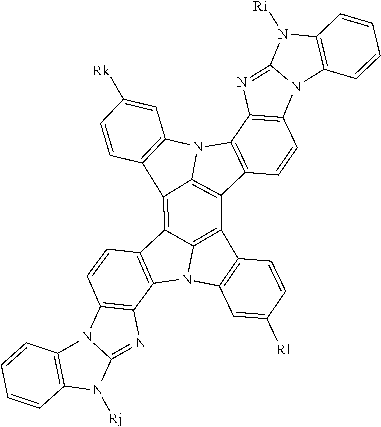 |
| Compound 312- (Ri)(Rj)(Rk)(Rl), wherein Compound 312- (R1)(R1)(R1)(R1) to Compound 312- (R468)(R468) (R468)(R468), have the structure | 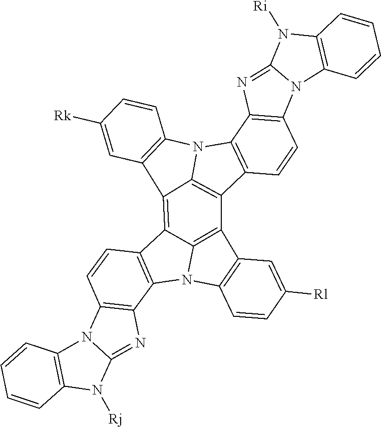 |
| Compound 313- (Ri)(Rj)(Rk)(Rl), wherein Compound 313- (R1)(R1)(R1)(R1) to Compound 313- (R468)(R468) (R468)(R468), have the structure | 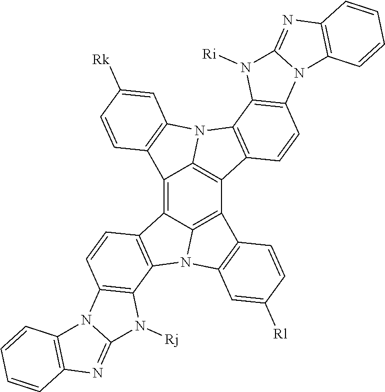 |
| Compound 314- (Ri)(Rj)(Rk)(Rl), wherein Compound 314- (R1)(R1)(R1)(R1) to Compound 314- (R468)(R468) (R468)(R468), have the structure | 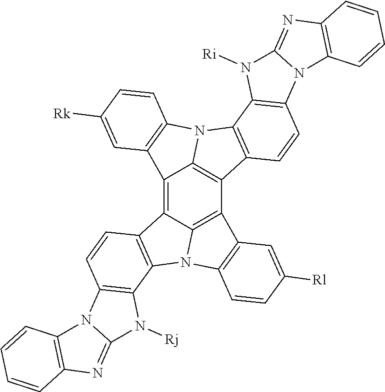 |
| Compound 315- (Ri)(Rj)(Rk)(Rl), wherein Compound 315- (R1)(R1)(R1)(R1) to Compound 315- (R468)(R468) (R468)(R468), have the structure | 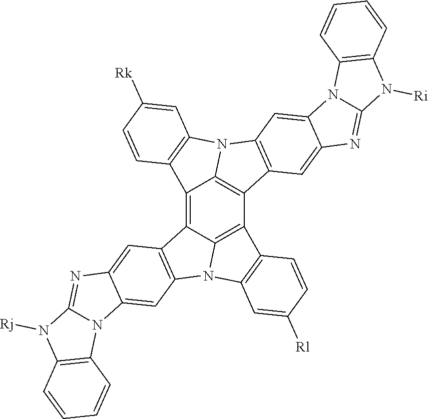 |
| Compound 316- (Ri)(Rj)(Rk)(Rl), wherein Compound 316- (R1)(R1)(R1)(R1) to Compound 316- (R468)(R468) (R468)(R468), have the structure | 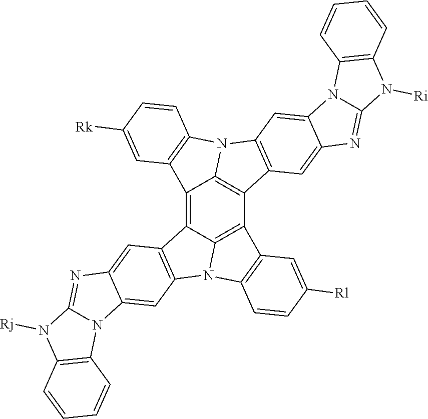 |
| Compound 317- (Ri)(Rj)(Rk)(Rl), wherein Compound 317- (R1)(R1)(R1)(R1) to Compound 317- (R468)(R468) (R468)(R468), have the structure | 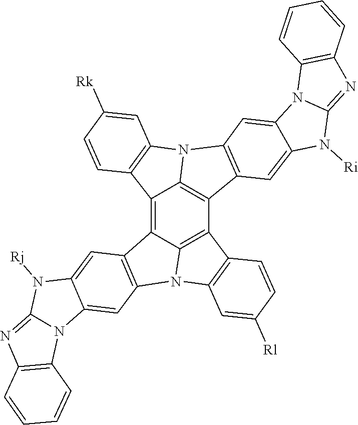 |
| Compound 318- (Ri)(Rj)(Rk)(Rl), wherein Compound 318- (R1)(R1)(R1)(R1) to Compound 318- (R468)(R468) (R468)(R468), have the structure | 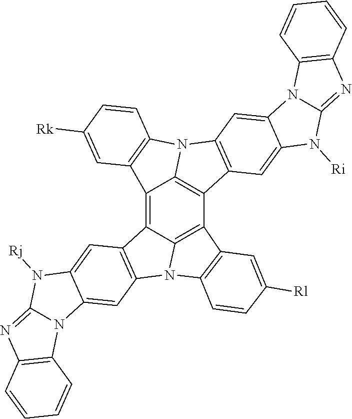 |
| Compound 319- (Ri)(Rj)(Rk)(Rl), wherein Compound 319- (R1)(R1)(R1)(R1) to Compound 319- (R468)(R468) (R468)(R468), have the structure | 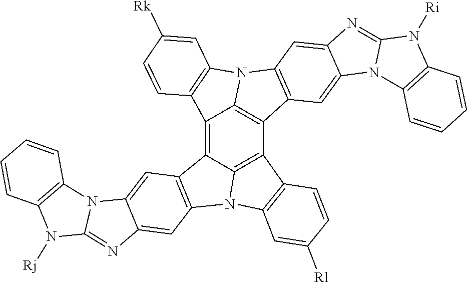 |
| Compound 320- (Ri)(Rj)(Rk)(Rl), wherein Compound 320- (R1)(R1)(R1)(R1) to Compound 320- (R468)(R468) (R468)(R468), have the structure | 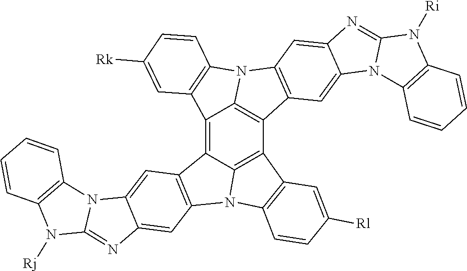 |
| Compound 321- (Ri)(Rj)(Rk)(Rl), wherein Compound 321- (R1)(R1)(R1)(R1) to Compound 321- (R468)(R468) (R468)(R468), have the structure | 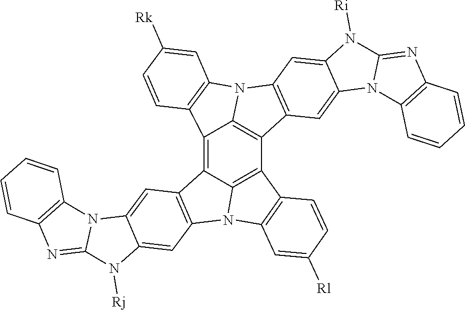 |
| Compound 322- (Ri)(Rj)(Rk)(Rl), wherein Compound 322- (R1)(R1)(R1)(R1) to Compound 322- (R468)(R468) (R468)(R468), have the structure | 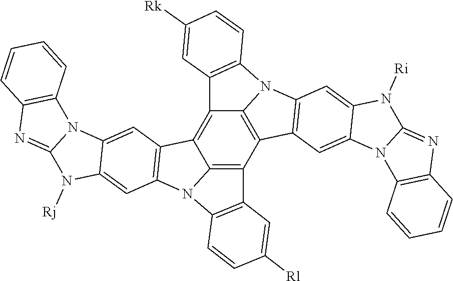 |
| Compound 323- (Ri)(Rj)(Rk)(Rl), wherein Compound 323- (R1)(R1)(R1)(R1) to Compound 323- (R468)(R468) (R468)(R468), have the structure | 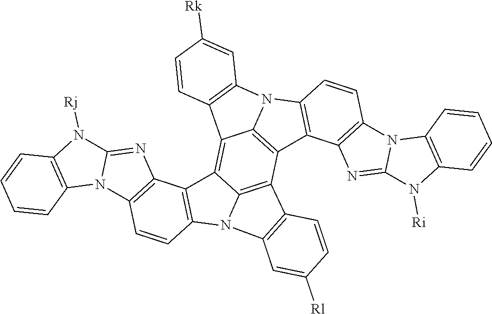 |
| Compound 324- (Ri)(Rj)(Rk)(Rl), wherein Compound 324- (R1)(R1)(R1)(R1) to Compound 324- (R468)(R468) (R468)(R468), have the structure | 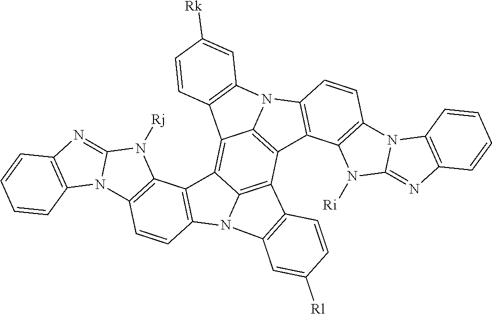 |
| Compound 325- (Ri)(Rj)(Rk)(Rl), wherein Compound 325- (R1)(R1)(R1)(R1) to Compound 325- (R468)(R468) (R468)(R468), have the structure | 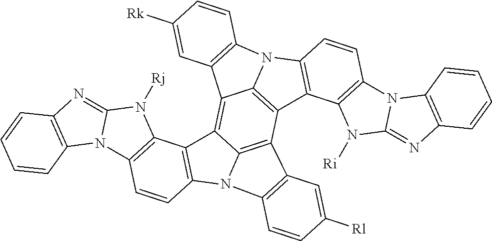 |
| Compound 326- (Ri)(Rj)(Rk)(Rl), wherein Compound 326- (R1)(R1)(R1)(R1) to Compound 326- (R468)(R468) (R468)(R468), have the structure | 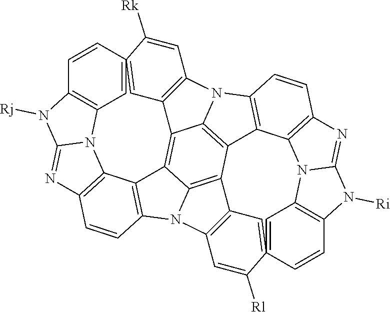 |
| Compound 327- (Ri)(Rj)(Rk)(Rl), wherein Compound 327- (R1)(R1)(R1)(R1) to Compound 327- (R468)(R468) (R468)(R468), have the structure | 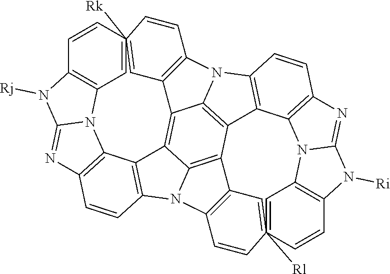 |
| Compound 328- (Ri)(Rj)(Rk)(Rl), wherein Compound 328- (R1)(R1)(R1)(R1) to Compound 328- (R468)(R468) (R468)(R468), have the structure | 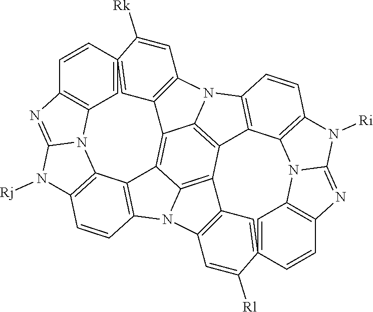 |
| Compound 329- (Ri)(Rj)(Rk)(Rl), wherein Compound 329- (R1)(R1)(R1)(R1) to Compound 329- (R468)(R468) (R468)(R468), have the structure | 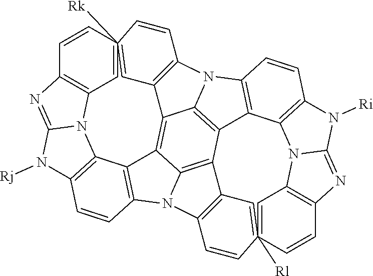 |
| Compound 330- (Ri)(Rj)(Rk)(Rl), wherein Compound 330- (R1)(R1)(R1)(R1) to Compound 330- (R468)(R468) (R468)(R468), have the structure | 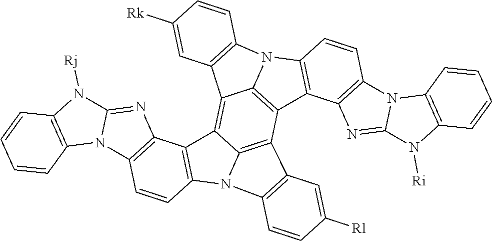 |
| Compound 331- (Ri)(Rj)(Rk)(Rl), wherein Compound 331- (R1)(R1)(R1)(R1) to Compound 331- (R468)(R468) (R468)(R468), have the structure | 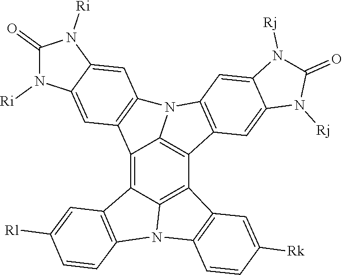 |
| Compound 332- (Ri)(Rj)(Rk)(Rl), wherein Compound 332- (R1)(R1)(R1)(R1) to Compound 332- (R468)(R468) (R468)(R468), have the structure | 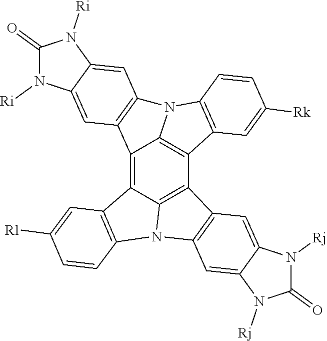 |
| Compound 333- (Ri)(Rj)(Rk)(Rl), wherein Compound 333- (R1)(R1)(R1)(R1) to Compound 333- (R468)(R468) (R468)(R468), have the structure | 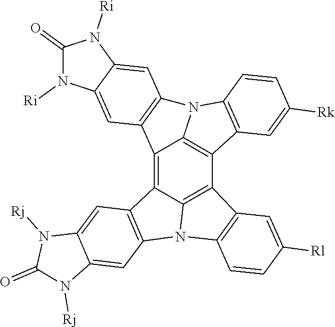 |
| Compound 334- (Ri)(Rj)(Rk)(Rl), wherein Compound 334- (R1)(R1)(R1)(R1) to Compound 334- (R468)(R468) (R468)(R468), have the structure | 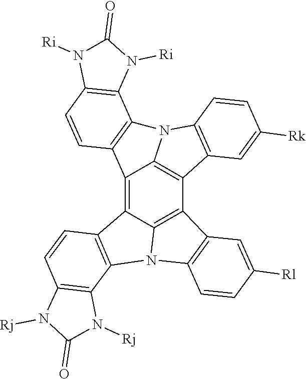 |
| Compound 335- (Ri)(Rj)(Rk)(Rl), wherein Compound 335- (R1)(R1)(R1)(R1) to Compound 335- (R468)(R468) (R468)(R468), have the structure | 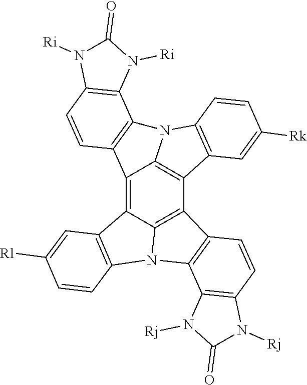 |
| Compound 336- (Ri)(Rj)(Rk)(Rl), wherein Compound 336- (R1)(R1)(R1)(R1) to Compound 336- (R468)(R468) (R468)(R468), have the structure | 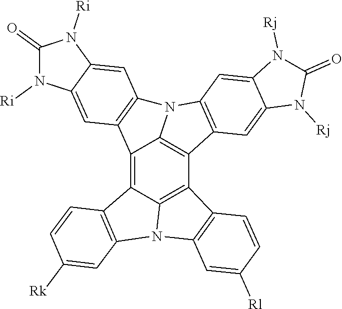 |
| Compound 337- (Ri)(Rj)(Rk)(Rl), wherein Compound 337- (R1)(R1)(R1)(R1) to Compound 337- (R468)(R468) (R468)(R468), have the structure | 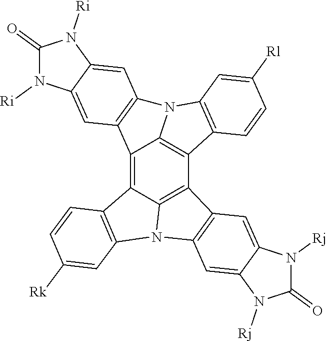 |
| Compound 338- (Ri)(Rj)(Rk)(Rl), wherein Compound 338- (R1)(R1)(R1)(R1) to Compound 338- (R468)(R468) (R468)(R468), have the structure | 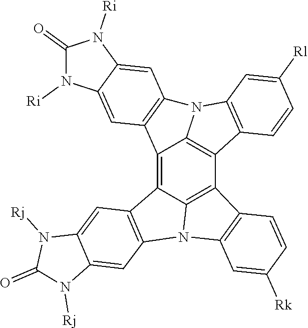 |
| Compound 339- (Ri)(Rj)(Rk)(Rl), wherein Compound 339- (R1)(R1)(R1)(R1) to Compound 339- (R468)(R468) (R468)(R468), have the structure | 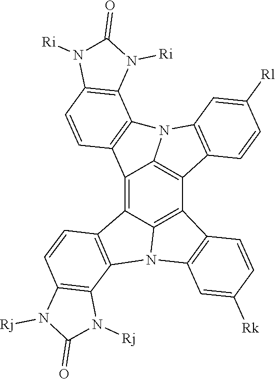 |
| Compound 340- (Ri)(Rj)(Rk)(Rl), wherein Compound 340- (R1)(R1)(R1)(R1) to Compound 340- (R468)(R468) (R468)(R468), have the structure | 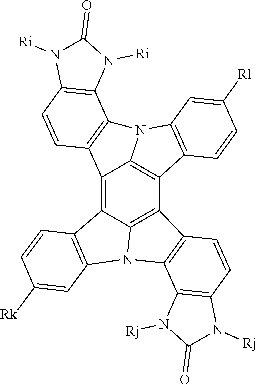 |
| Compound 341- (Ri)(Rj)(Rk)(Rl), wherein Compound 341- (R1)(R1)(R1)(R1) to Compound 341- (R468)(R468) (R468)(R468), have the structure | 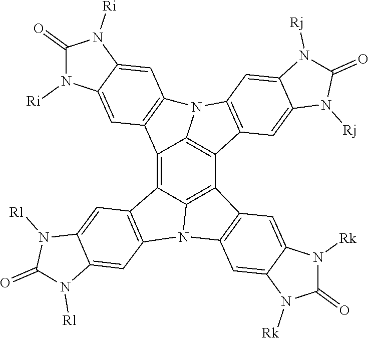 |
| Compound 342- (Ri)(Rj)(Rn′), wherein Compound 342- (R1)(R1)(R1) to Compound 342- (R468)(R468) (R468), have the structure | 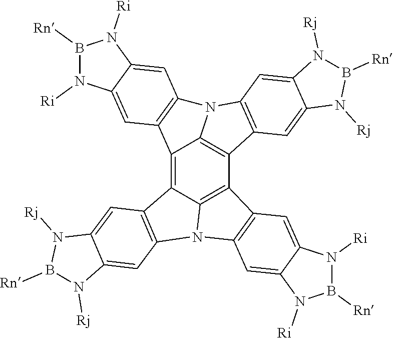 |

wherein Y1 to Y51 have the following structures as defined in the following LIST 15:

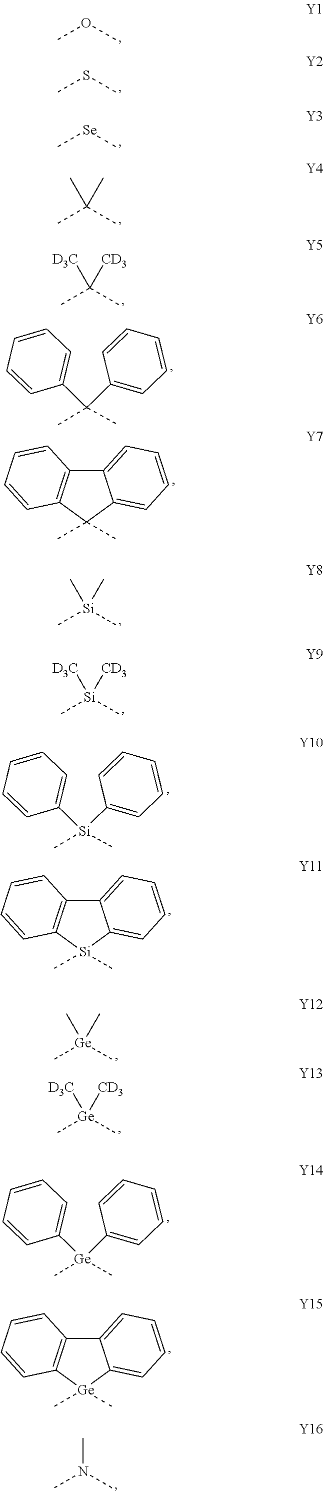

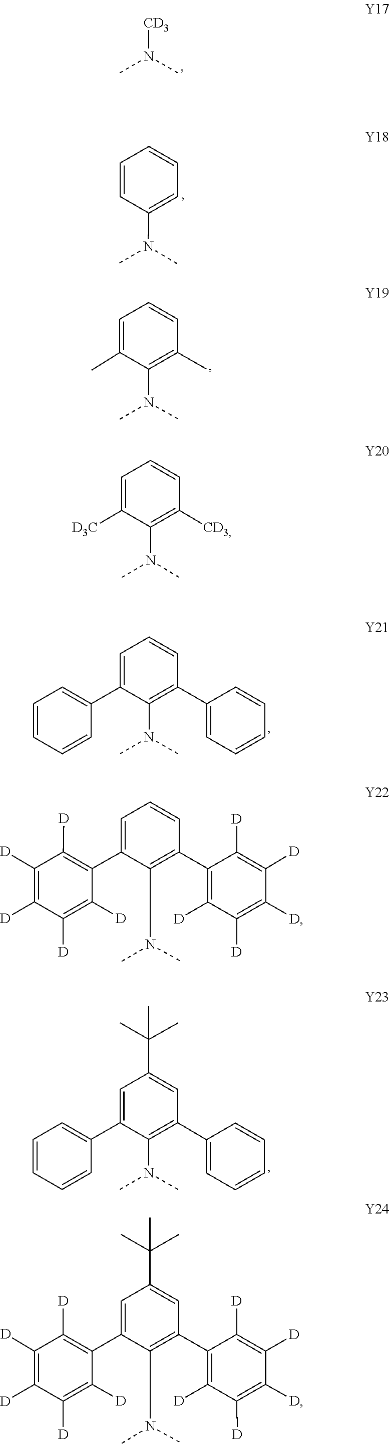

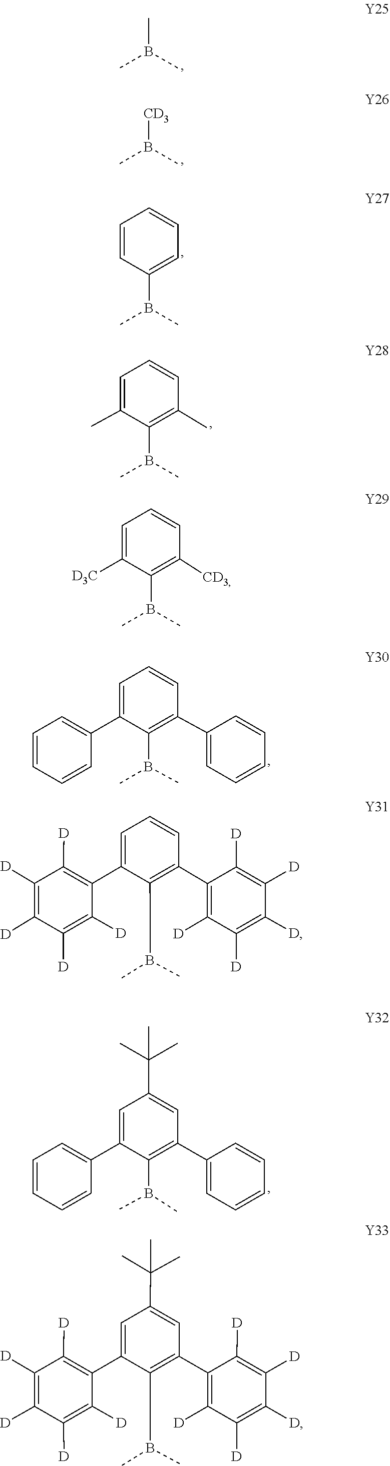

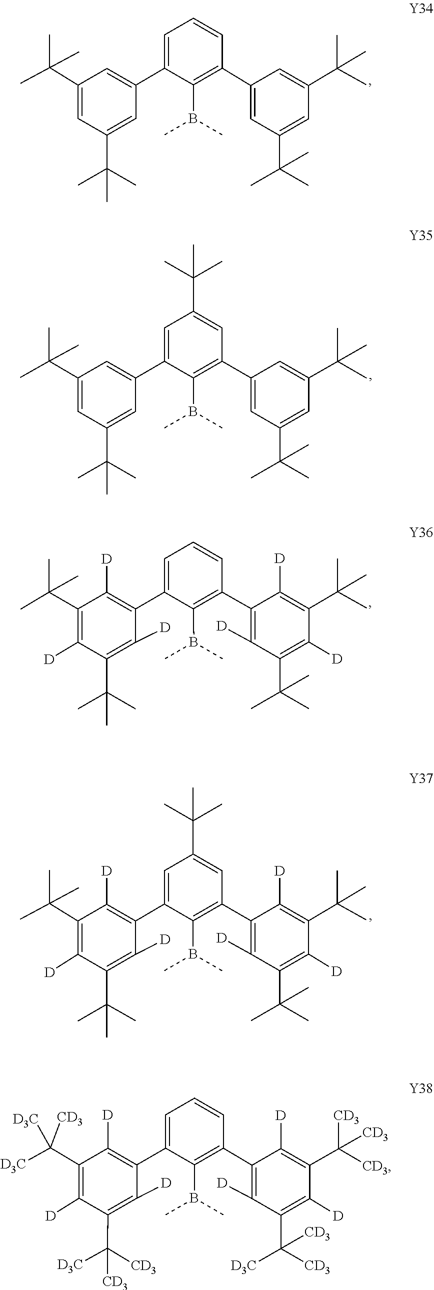

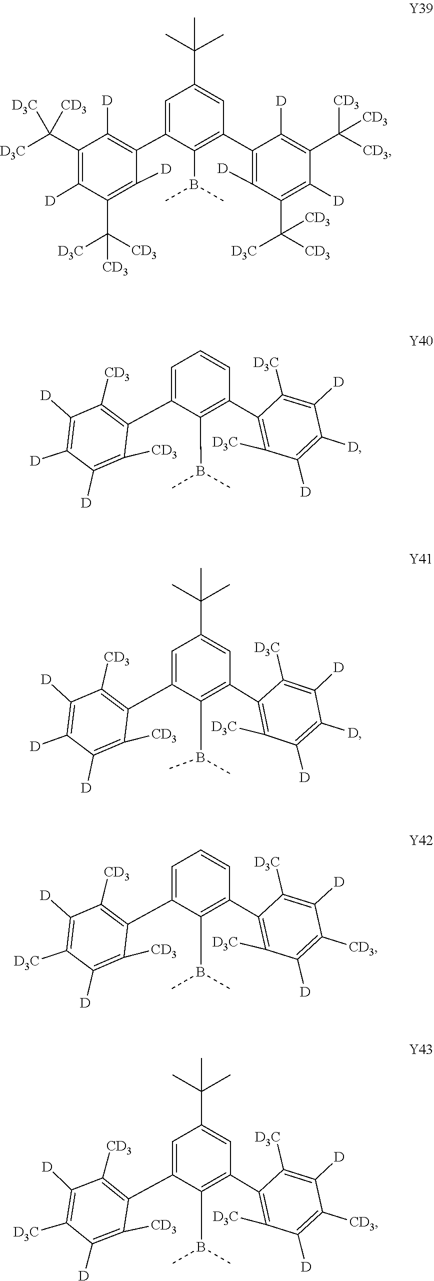

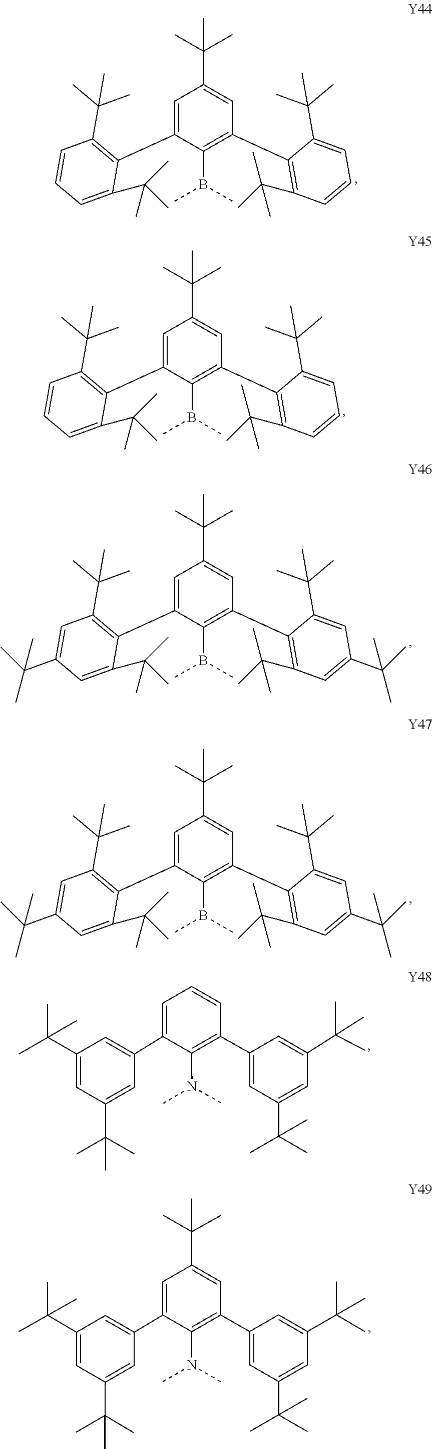

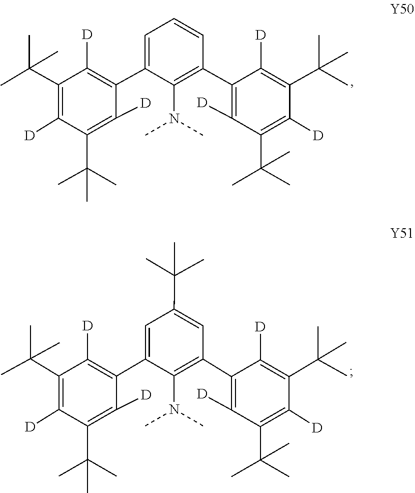

wherein R1 to R468 have the following structures (LIST 3):

| Structure | |
|---|---|
| 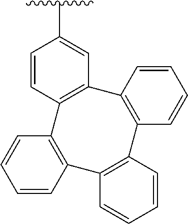 [SMILES: *c1ccc2c(c1)-c1ccccc1-c1ccccc1-c1ccccc1-2] | R1 |
| 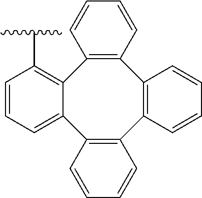 [SMILES: *c1cccc2c1-c1ccccc1-c1ccccc1-c1ccccc1-2] | R2 |
| 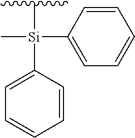 [SMILES: *[Si](C)(c1ccccc1)c1ccccc1] | R3 |
| 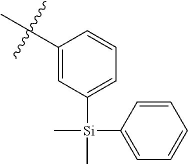 [SMILES: *c1cccc([Si](C)(C)c2ccccc2)c1] | R4 |
| 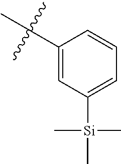 [SMILES: *c1cccc([Si](C)(C)C)c1] | R5 |
| 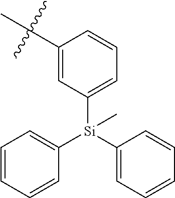 [SMILES: *c1cccc([Si](C)(c2ccccc2)c2ccccc2)c1] | R6 |
| 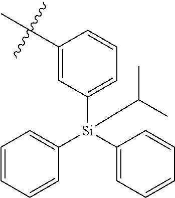 [SMILES: *c1cccc([Si](c2ccccc2)(c2ccccc2)C(C)C)c1] | R7 |
| 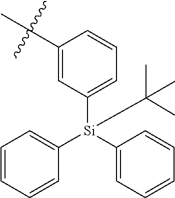 [SMILES: *c1cccc([Si](c2ccccc2)(c2ccccc2)C(C)(C)C)c1] | R8 |
| 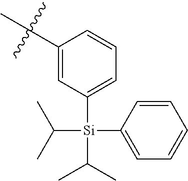 [SMILES: *c1cccc([Si](c2ccccc2)(C(C)C)C(C)C)c1] | R9 |
| 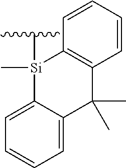 | R10 |
| 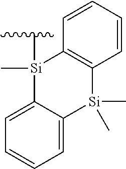 | R11 |
| 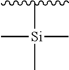 | R12 |
| 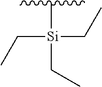 [SMILES: *[Si](CC)(CC)CC] | R13 |
| 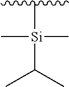 | R14 |
| 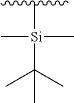 | R15 |
| 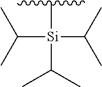 [SMILES: *[Si](C(C)C)(C(C)C)C(C)C] | R16 |
| 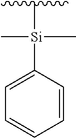 | R17 |
| 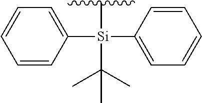 [SMILES: *[Si](c1ccccc1)(c1ccccc1)C(C)(C)C] | R18 |
| 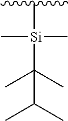 | R19 |
| 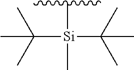 [SMILES: *[Si](C)(C(C)(C)C)C(C)(C)C] | R20 |
| 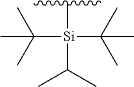 [SMILES: *[Si](C(C)C)(C(C)(C)C)C(C)(C)C] | R21 |
| 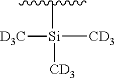 [SMILES: *[Si](C([2H])([2H])[2H])(C([2H])([2H])[2H])C([2H])([2H])[2H]] | R22 |
| 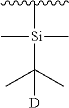 | R23 |
| 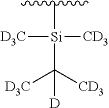 [SMILES: *[Si](C([2H])([2H])[2H])(C([2H])([2H])[2H])C([2H])(C([2H])([2H])[2H])C([2H])([2H])[2H]] | R24 |
| 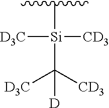 [SMILES: *[Si](C([2H])([2H])[2H])(C([2H])([2H])[2H])C([2H])(C([2H])([2H])[2H])C([2H])([2H])[2H]] | R25 |
| 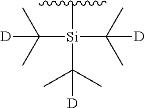 [SMILES: *[Si](C([2H])(C)C)(C([2H])(C)C)C([2H])(C)C] | R26 |
| 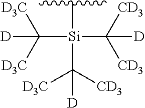 [SMILES: *[Si](C([2H])(C([2H])([2H])[2H])C([2H])([2H])[2H])(C([2H])(C([2H])([2H])[2H])C([2H])([2H])[2H])C([2H])(C([2H])([2H])[2H])C([2H])([2H])[2H]] | R27 |
| 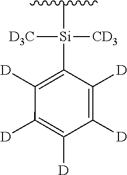 [SMILES: *[Si](c1c([2H])c([2H])c([2H])c([2H])c1[2H])(C([2H])([2H])[2H])C([2H])([2H])[2H]] | R28 |
| 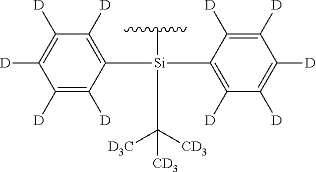 | R29 |
| 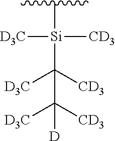 [SMILES: *[Si](C([2H])([2H])[2H])(C([2H])([2H])[2H])C(C([2H])([2H])[2H])(C([2H])([2H])[2H])C([2H])(C([2H])([2H])[2H])C([2H])([2H])[2H]] | R30 |
| 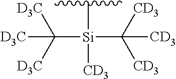 [SMILES: *[Si](C([2H])([2H])[2H])(C(C([2H])([2H])[2H])(C([2H])([2H])[2H])C([2H])([2H])[2H])C(C([2H])([2H])[2H])(C([2H])([2H])[2H])C([2H])([2H])[2H]] | R31 |
| 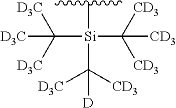 [SMILES: *[Si](C([2H])(C([2H])([2H])[2H])C([2H])([2H])[2H])(C(C([2H])([2H])[2H])(C([2H])([2H])[2H])C([2H])([2H])[2H])C(C([2H])([2H])[2H])(C([2H])([2H])[2H])C([2H])([2H])[2H]] | R32 |
| 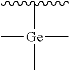 | R33 |
| 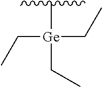 | R34 |
| 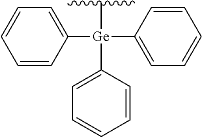 | R35 |
| 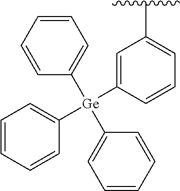 | R36 |
| 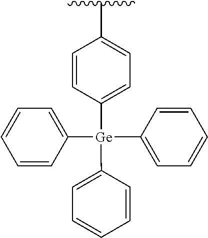 | R37 |
| 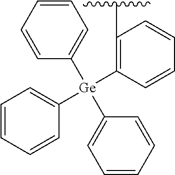 | R38 |
| 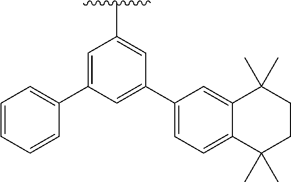 [SMILES: *c1cc(-c2ccccc2)cc(-c2ccc3c(c2)C(C)(C)CCC3(C)C)c1] | R39 |
| 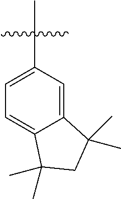 [SMILES: *c1ccc2c(c1)C(C)(C)CC2(C)C] | R40 |
| 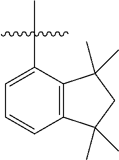 [SMILES: *c1cccc2c1C(C)(C)CC2(C)C] | R41 |
| 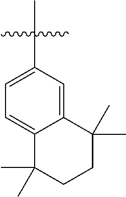 | R42 |
| 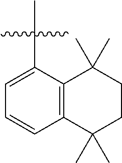 [SMILES: *c1cccc2c1C(C)(C)CCC2(C)C] | R43 |
| 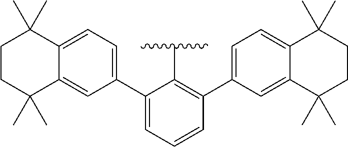 [SMILES: *c1c(-c2ccc3c(c2)C(C)(C)CCC3(C)C)cccc1-c1ccc2c(c1)C(C)(C)CCC2(C)C] | R44 |
| 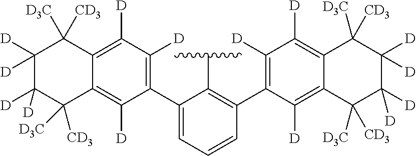 [SMILES: *c1c(-c2c([2H])c([2H])c3c(c2[2H])C(C([2H])([2H])[2H])(C([2H])([2H])[2H])C([2H])([2H])C([2H])([2H])C3(C([2H])([2H])[2H])C([2H])([2H])[2H])cccc1-c1c([2H])c([2H])c2c(c1[2H])C(C([2H])([2H])[2H])(C([2H])([2H])[2H])C([2H])([2H])C([2H])([2H])C2(C([2H])([2H])[2H])C([2H])([2H])[2H]] | R45 |
| 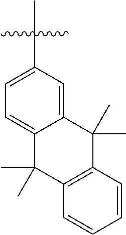 | R46 |
| 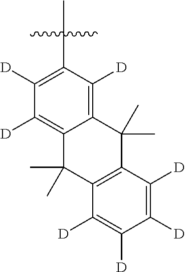 [SMILES: *c1c([2H])c([2H])c2c(c1[2H])C(C)(C)c1c([2H])c([2H])c([2H])c([2H])c1C2(C)C] | R47 |
| 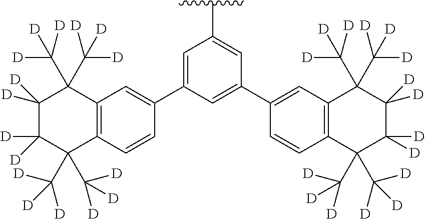 [SMILES: *c1cc(-c2ccc3c(c2)C(C([2H])([2H])[2H])(C([2H])([2H])[2H])C([2H])([2H])C([2H])([2H])C3(C([2H])([2H])[2H])C([2H])([2H])[2H])cc(-c2ccc3c(c2)C(C([2H])([2H])[2H])(C([2H])([2H])[2H])C([2H])([2H])C([2H])([2H])C3(C([2H])([2H])[2H])C([2H])([2H])[2H])c1] | R48 |
| 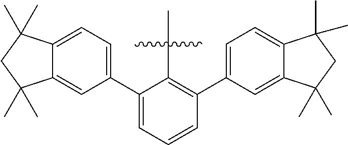 [SMILES: *c1c(-c2ccc3c(c2)C(C)(C)CC3(C)C)cccc1-c1ccc2c(c1)C(C)(C)CC2(C)C] | R49 |
| 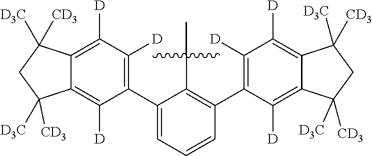 [SMILES: *c1c(-c2c([2H])c([2H])c3c(c2[2H])C(C([2H])([2H])[2H])(C([2H])([2H])[2H])CC3(C([2H])([2H])[2H])C([2H])([2H])[2H])cccc1-c1c([2H])c([2H])c2c(c1[2H])C(C([2H])([2H])[2H])(C([2H])([2H])[2H])CC2(C([2H])([2H])[2H])C([2H])([2H])[2H]] | R50 |
| 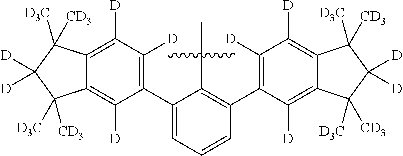 [SMILES: *c1c(-c2c([2H])c([2H])c3c(c2[2H])C(C([2H])([2H])[2H])(C([2H])([2H])[2H])C([2H])([2H])C3(C([2H])([2H])[2H])C([2H])([2H])[2H])cccc1-c1c([2H])c([2H])c2c(c1[2H])C(C([2H])([2H])[2H])(C([2H])([2H])[2H])C([2H])([2H])C2(C([2H])([2H])[2H])C([2H])([2H])[2H]] | R51 |
| 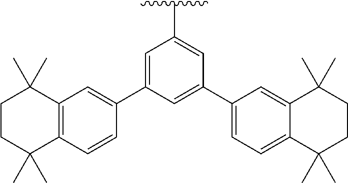 [SMILES: *c1cc(-c2ccc3c(c2)C(C)(C)CCC3(C)C)cc(-c2ccc3c(c2)C(C)(C)CCC3(C)C)c1] | R52 |
| 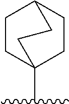 [SMILES: *C12CCC(CC1)CC2] | R53 |
| 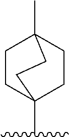 [SMILES: *C12CCC(C)(CC1)CC2] | R54 |
| 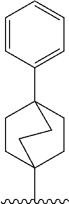 [SMILES: *C12CCC(c3ccccc3)(CC1)CC2] | R55 |
| 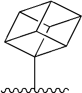 [SMILES: *C12C3C4C5C3C1C5C42] | R56 |
| 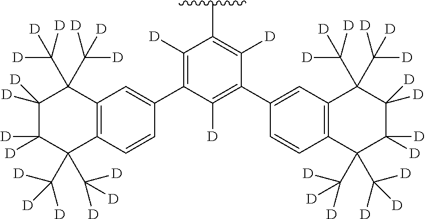 [SMILES: *c1c([2H])c(-c2ccc3c(c2)C(C([2H])([2H])[2H])(C([2H])([2H])[2H])C([2H])([2H])C([2H])([2H])C3(C([2H])([2H])[2H])C([2H])([2H])[2H])c([2H])c(-c2ccc3c(c2)C(C([2H])([2H])[2H])(C([2H])([2H])[2H])C([2H])([2H])C([2H])([2H])C3(C([2H])([2H])[2H])C([2H])([2H])[2H])c1[2H]] | R57 |
| 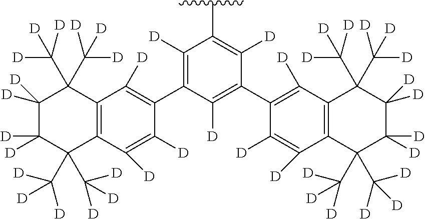 [SMILES: *c1c([2H])c(-c2c([2H])c([2H])c3c(c2[2H])C(C([2H])([2H])[2H])(C([2H])([2H])[2H])C([2H])([2H])C([2H])([2H])C3(C([2H])([2H])[2H])C([2H])([2H])[2H])c([2H])c(-c2c([2H])c([2H])c3c(c2[2H])C(C([2H])([2H])[2H])(C([2H])([2H])[2H])C([2H])([2H])C([2H])([2H])C3(C([2H])([2H])[2H])C([2H])([2H])[2H])c1[2H]] | R58 |
| 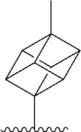 [SMILES: *C12C3C4C1C1C2C3C41C] | R59 |
| 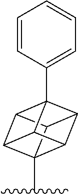 [SMILES: *C12C3C4C1C1C2C3C41c1ccccc1] | R60 |
| 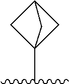 [SMILES: *C12CC(C1)C2] | R61 |
| 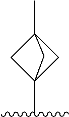 [SMILES: *C12CC(C)(C1)C2] | R62 |
| 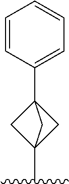 [SMILES: *C12CC(c3ccccc3)(C1)C2] | R63 |
| 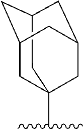 [SMILES: *C12CC3CC(CC(C3)C1)C2] | R64 |
| 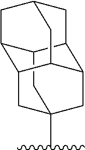 [SMILES: *C12CC3C4CC5CC3C(C1)C(C5)C4C2] | R65 |
| 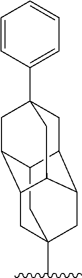 [SMILES: *C12CC3C4CC5(c6ccccc6)CC3C(C1)C(C5)C4C2] | R66 |
| 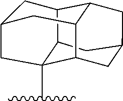 [SMILES: *C12CC3CC4C5CC(CC41)CC2C5C3] | R67 |
| 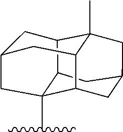 [SMILES: *C12CC3CC4C1CC1CC2C(C3)C4(C)C1] | R68 |
| 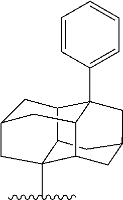 [SMILES: *C12CC3CC4C1CC1CC2C(C3)C4(c2ccccc2)C1] | R69 |
| 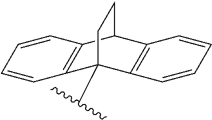 [SMILES: *C12CCC(c3ccccc31)c1ccccc12] | R70 |
| 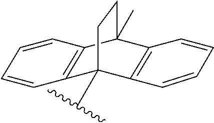 [SMILES: *C12CCC(C)(c3ccccc31)c1ccccc12] | R71 |
| 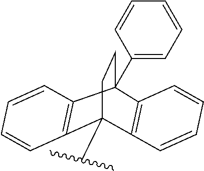 [SMILES: *C1c2ccccc2C(CC)(c2ccccc2)c2ccccc21] | R72 |
| 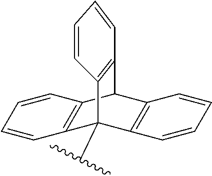 [SMILES: *C12c3ccccc3C(c3ccccc31)c1ccccc12] | R73 |
| 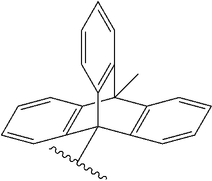 [SMILES: *C12c3ccccc3C(C)(c3ccccc31)c1ccccc12] | R74 |
| 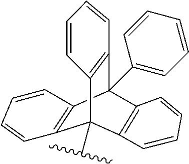 [SMILES: *C12c3ccccc3C(c3ccccc3)(c3ccccc31)c1ccccc12] | R75 |
| 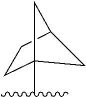 [SMILES: *C12CCC(C1)C2] | R76 |
| 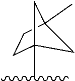 [SMILES: *C12CCC(C)(C1)C2] | R77 |
| 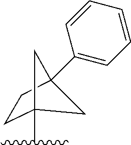 [SMILES: *C12CCC(c3ccccc3)(C1)C2] | R78 |
| 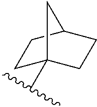 [SMILES: *C12CCC(CC1)C2] | R79 |
| 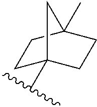 [SMILES: *C12CCC(C)(CC1)C2] | R80 |
| 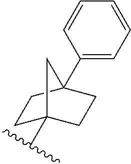 [SMILES: *C12CCC(c3ccccc3)(CC1)C2] | R81 |
| 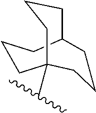 [SMILES: *C12CCCC(CCC1)CCC2] | R82 |
| 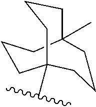 [SMILES: *C12CCCC(C)(CCC1)CCC2] | R83 |
| 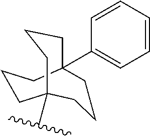 [SMILES: *C12CCCC(c3ccccc3)(CCC1)CCC2] | R84 |
| 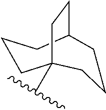 [SMILES: *C12CCCC(CCC1)CC2] | R88 |
| 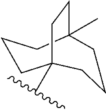 [SMILES: *C12CCCC(C)(CCC1)CC2] | R89 |
| 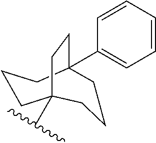 [SMILES: *C12CCCC(c3ccccc3)(CCC1)CC2] | R90 |
| 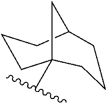 [SMILES: *C12CCCC(CCC1)C2] | R91 |
| 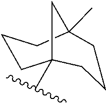 [SMILES: *C12CCCC(C)(CCC1)C2] | R92 |
| 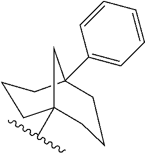 [SMILES: *C12CCCC(c3ccccc3)(CCC1)C2] | R93 |
| 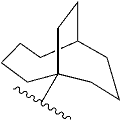 [SMILES: *C12CCCCC(CCC1)CC2] | R94 |
| 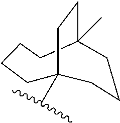 [SMILES: *C12CCCCC(C)(CCC1)CC2] | R95 |
| 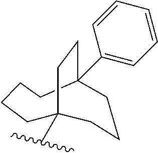 [SMILES: *C12CCCCC(c3ccccc3)(CCC1)CC2] | R96 |
| 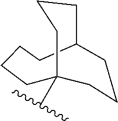 [SMILES: *C12CCCCC(CCC1)CCC2] | R97 |
| 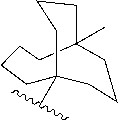 [SMILES: *C12CCCCC(C)(CCC1)CCC2] | R98 |
| 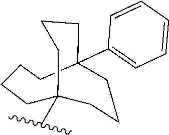 [SMILES: *C12CCCCC(c3ccccc3)(CCC1)CCC2] | R99 |
| 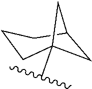 [SMILES: *C12CCCC(C1)C2] | R100 |
| 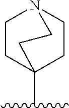 [SMILES: *C12CCN(CC1)CC2] | R101 |
| 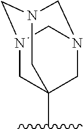 [SMILES: *C12CN3CN(CN(C3)C1)C2] | R102 |
| 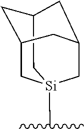 [SMILES: *[Si]12CC3CC(CC(C3)C1)C2] | R103 |
| 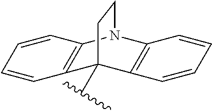 [SMILES: *C12CCN(c3ccccc31)c1ccccc12] | R104 |
| 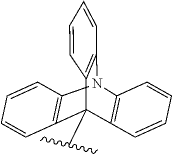 [SMILES: *C1c2ccccc2N(c2ccccc2)c2ccccc21] | R105 |
| 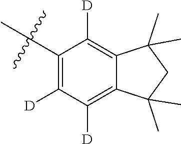 [SMILES: *c1c([2H])c([2H])c2c(c1[2H])C(C)(C)CC2(C)C] | R106 |
| 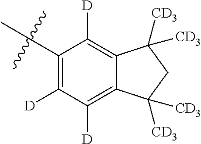 [SMILES: *c1c([2H])c([2H])c2c(c1[2H])C(C([2H])([2H])[2H])(C([2H])([2H])[2H])CC2(C([2H])([2H])[2H])C([2H])([2H])[2H]] | R107 |
| 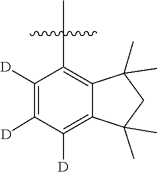 [SMILES: *c1c([2H])c([2H])c([2H])c2c1C(C)(C)CC2(C)C] | R108 |
| 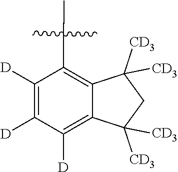 [SMILES: *c1c([2H])c([2H])c([2H])c2c1C(C([2H])([2H])[2H])(C([2H])([2H])[2H])CC2(C([2H])([2H])[2H])C([2H])([2H])[2H]] | R109 |
| 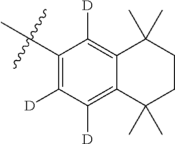 [SMILES: *c1c([2H])c([2H])c2c(c1[2H])C(C)(C)CCC2(C)C] | R110 |
| 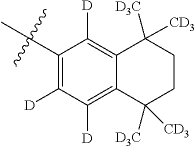 [SMILES: *c1c([2H])c([2H])c2c(c1[2H])C(C([2H])([2H])[2H])(C([2H])([2H])[2H])CCC2(C([2H])([2H])[2H])C([2H])([2H])[2H]] | R111 |
| 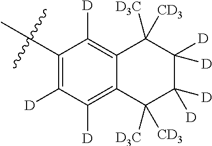 [SMILES: *c1c([2H])c([2H])c2c(c1[2H])C(C([2H])([2H])[2H])(C([2H])([2H])[2H])C([2H])([2H])C([2H])([2H])C2(C([2H])([2H])[2H])C([2H])([2H])[2H]] | R112 |
| 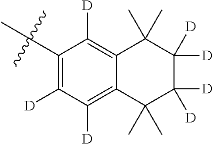 [SMILES: *c1c([2H])c([2H])c2c(c1[2H])C(C)(C)C([2H])([2H])C([2H])([2H])C2(C)C] | R113 |
| 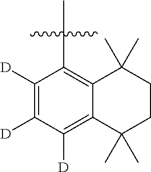 [SMILES: *c1c([2H])c([2H])c([2H])c2c1C(C)(C)CCC2(C)C] | R114 |
| 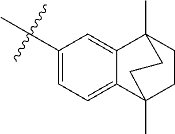 [SMILES: *c1ccc2c(c1)C1(C)CCC2(C)CC1] | R115 |
| 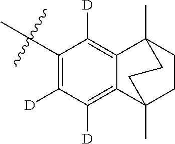 [SMILES: *c1c([2H])c([2H])c2c(c1[2H])C1(C)CCC2(C)CC1] | R116 |
| 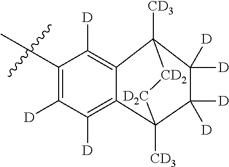 | R117 |
| 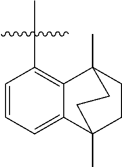 [SMILES: *c1cccc2c1C1(C)CCC2(C)CC1] | R118 |
| 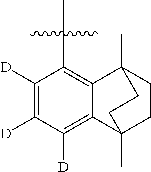 [SMILES: *c1c([2H])c([2H])c([2H])c2c1C1(C)CCC2(C)CC1] | R119 |
| 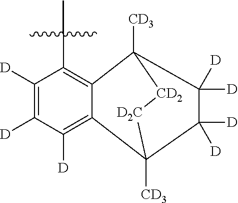 | R120 |
| 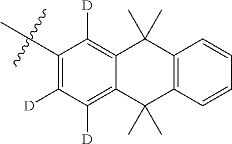 [SMILES: *c1c([2H])c([2H])c2c(c1[2H])C(C)(C)c1ccccc1C2(C)C] | R121 |
| 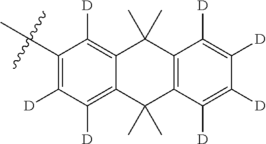 [SMILES: *c1c([2H])c([2H])c2c(c1[2H])C(C)(C)c1c([2H])c([2H])c([2H])c([2H])c1C2(C)C] | R122 |
| 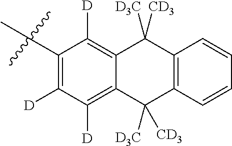 [SMILES: *c1c([2H])c([2H])c2c(c1[2H])C(C([2H])([2H])[2H])(C([2H])([2H])[2H])c1ccccc1C2(C([2H])([2H])[2H])C([2H])([2H])[2H]] | R123 |
| 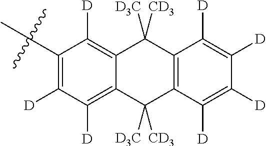 [SMILES: *c1c([2H])c([2H])c2c(c1[2H])C(C([2H])([2H])[2H])(C([2H])([2H])[2H])c1c([2H])c([2H])c([2H])c([2H])c1C2(C([2H])([2H])[2H])C([2H])([2H])[2H]] | R124 |
| 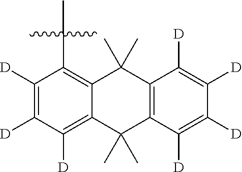 [SMILES: *c1c([2H])c([2H])c([2H])c2c1C(C)(C)c1c([2H])c([2H])c([2H])c([2H])c1C2(C)C] | R125 |
| 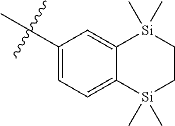 [SMILES: *c1ccc2c(c1)[Si](C)(C)CC[Si]2(C)C] | R126 |
| 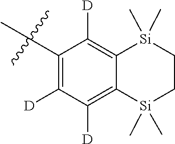 [SMILES: *c1c([2H])c([2H])c2c(c1[2H])[Si](C)(C)CC[Si]2(C)C] | R127 |
| 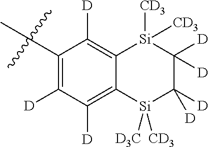 [SMILES: *c1c([2H])c([2H])c2c(c1[2H])[Si](C([2H])([2H])[2H])(C([2H])([2H])[2H])C([2H])([2H])C([2H])([2H])[Si]2(C([2H])([2H])[2H])C([2H])([2H])[2H]] | R128 |
| 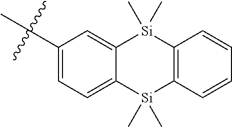 [SMILES: *c1ccc2c(c1)[Si](C)(C)c1ccccc1[Si]2(C)C] | R129 |
| 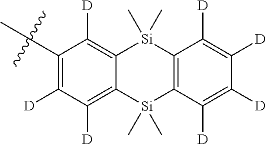 [SMILES: *c1c([2H])c([2H])c2c(c1[2H])[Si](C)(C)c1c([2H])c([2H])c([2H])c([2H])c1[Si]2(C)C] | R130 |
| 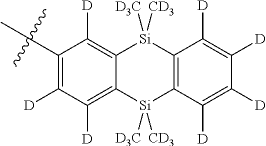 [SMILES: *c1c([2H])c([2H])c2c(c1[2H])[Si](C([2H])([2H])[2H])(C([2H])([2H])[2H])c1c([2H])c([2H])c([2H])c([2H])c1[Si]2(C([2H])([2H])[2H])C([2H])([2H])[2H]] | R131 |
| 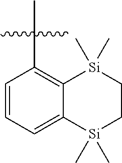 [SMILES: *c1cccc2c1[Si](C)(C)CC[Si]2(C)C] | R132 |
| 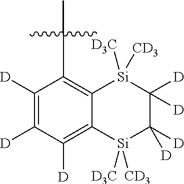 [SMILES: *c1c([2H])c([2H])c([2H])c2c1[Si](C([2H])([2H])[2H])(C([2H])([2H])[2H])C([2H])([2H])C([2H])([2H])[Si]2(C([2H])([2H])[2H])C([2H])([2H])[2H]] | R133 |
| 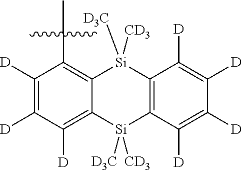 [SMILES: *c1c([2H])c([2H])c([2H])c2c1[Si](C([2H])([2H])[2H])(C([2H])([2H])[2H])c1c([2H])c([2H])c([2H])c([2H])c1[Si]2(C([2H])([2H])[2H])C([2H])([2H])[2H]] | R134 |
| 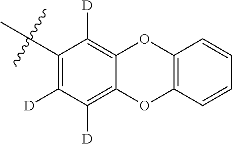 [SMILES: *c1c([2H])c([2H])c2c(c1[2H])Oc1ccccc1O2] | R135 |
| 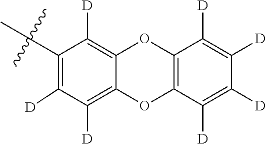 [SMILES: *c1c([2H])c([2H])c2c(c1[2H])Oc1c([2H])c([2H])c([2H])c([2H])c1O2] | R136 |
| 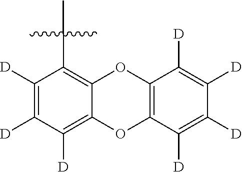 [SMILES: *c1c([2H])c([2H])c([2H])c2c1Oc1c([2H])c([2H])c([2H])c([2H])c1O2] | R137 |
| 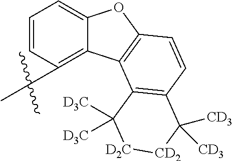 | R138 |
| 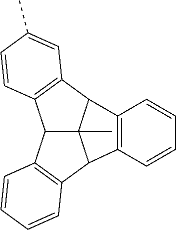 | R139 |
| 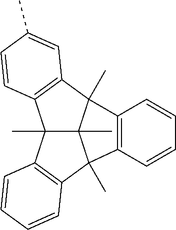 | R140 |
| 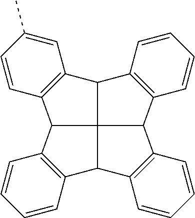 | R141 |
| 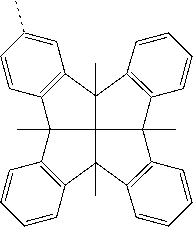 | R142 |
| 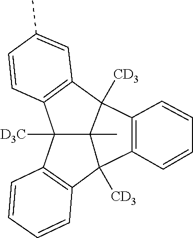 | R143 |
| 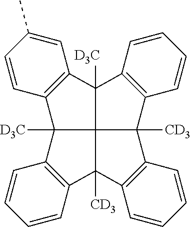 | R144 |
| 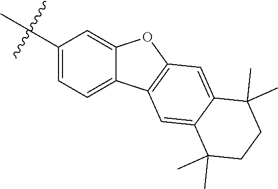 [SMILES: *c1ccc2c(c1)oc1cc3c(cc12)C(C)(C)CCC3(C)C] | R145 |
| 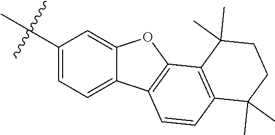 [SMILES: *c1ccc2c(c1)oc1c3c(ccc12)C(C)(C)CCC3(C)C] | R146 |
| 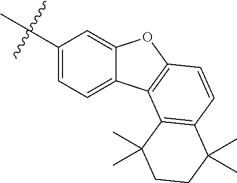 [SMILES: *c1ccc2c(c1)oc1ccc3c(c12)C(C)(C)CCC3(C)C] | R147 |
| 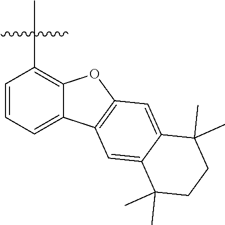 [SMILES: *c1cccc2c1oc1cc3c(cc12)C(C)(C)CCC3(C)C] | R148 |
| 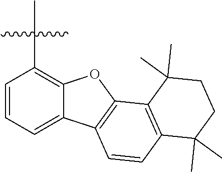 [SMILES: *c1cccc2c1oc1c3c(ccc12)C(C)(C)CCC3(C)C] | R149 |
| 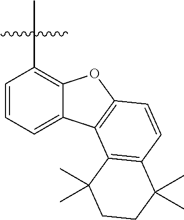 [SMILES: *c1cccc2c1oc1ccc3c(c12)C(C)(C)CCC3(C)C] | R150 |
| 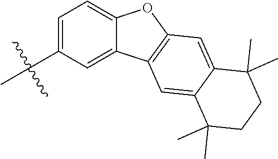 [SMILES: *c1ccc2oc3cc4c(cc3c2c1)C(C)(C)CCC4(C)C] | R151 |
| 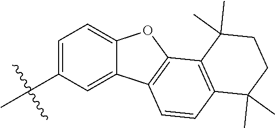 [SMILES: *c1ccc2oc3c4c(ccc3c2c1)C(C)(C)CCC4(C)C] | R152 |
| 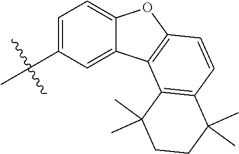 [SMILES: *c1ccc2oc3ccc4c(c3c2c1)C(C)(C)CCC4(C)C] | R153 |
| 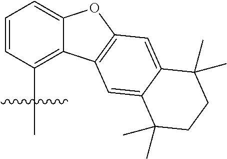 [SMILES: *c1cccc2oc3cc4c(cc3c12)C(C)(C)CCC4(C)C] | R154 |
| 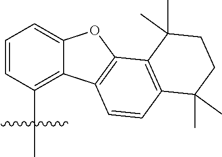 [SMILES: *c1cccc2oc3c4c(ccc3c12)C(C)(C)CCC4(C)C] | R155 |
| 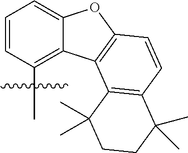 [SMILES: *c1cccc2oc3ccc4c(c3c12)C(C)(C)CCC4(C)C] | R156 |
| 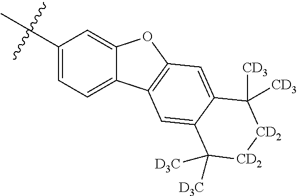 | R157 |
| 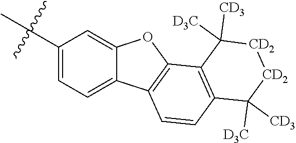 | R158 |
| 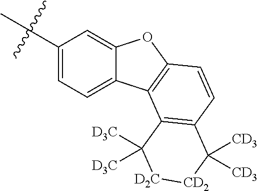 | R159 |
| 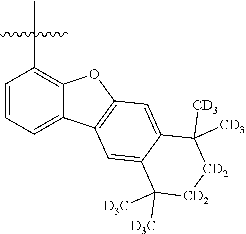 | R160 |
| 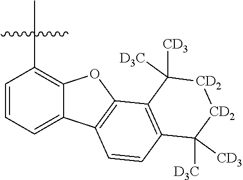 | R161 |
| 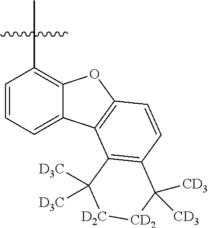 | R162 |
| 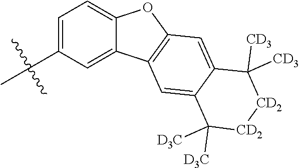 | R163 |
| 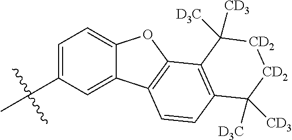 | R164 |
| 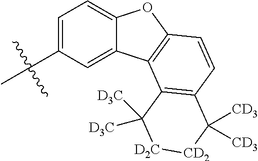 | R165 |
| 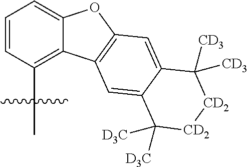 | R166 |
| 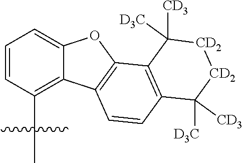 | R167 |
| 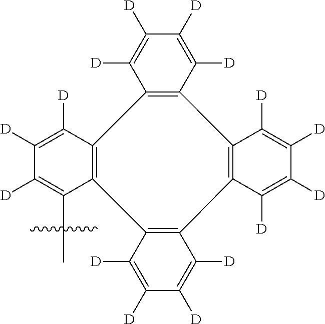 [SMILES: *c1c([2H])c([2H])c([2H])c2c1-c1c([2H])c([2H])c([2H])c([2H])c1-c1c([2H])c([2H])c([2H])c([2H])c1-c1c([2H])c([2H])c([2H])c([2H])c1-2] | R168 |
| 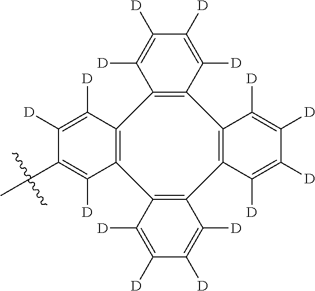 [SMILES: *c1c([2H])c([2H])c2c(c1[2H])-c1c([2H])c([2H])c([2H])c([2H])c1-c1c([2H])c([2H])c([2H])c([2H])c1-c1c([2H])c([2H])c([2H])c([2H])c1-2] | R169 |
| 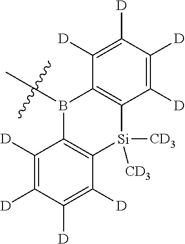 [SMILES: *B1c2c([2H])c([2H])c([2H])c([2H])c2[Si](C([2H])([2H])[2H])(C([2H])([2H])[2H])c2c([2H])c([2H])c([2H])c([2H])c21] | R170 |
| 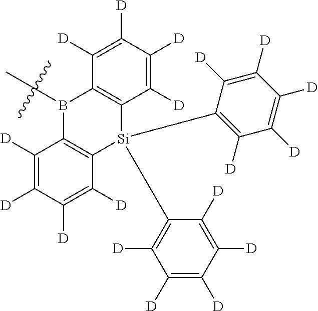 [SMILES: *B1c2c([2H])c([2H])c([2H])c([2H])c2[Si](c2c([2H])c([2H])c([2H])c([2H])c2[2H])(c2c([2H])c([2H])c([2H])c([2H])c2[2H])c2c([2H])c([2H])c([2H])c([2H])c21] | R171 |
| 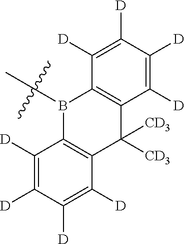 [SMILES: *B1c2c([2H])c([2H])c([2H])c([2H])c2C(C([2H])([2H])[2H])(C([2H])([2H])[2H])c2c([2H])c([2H])c([2H])c([2H])c21] | R172 |
| 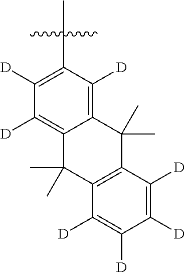 [SMILES: *c1c([2H])c([2H])c2c(c1[2H])C(C)(C)c1c([2H])c([2H])c([2H])c([2H])c1C2(C)C] | R173 |
| 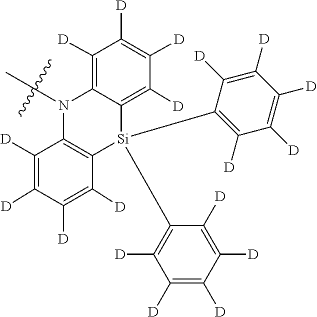 [SMILES: *N1c2c([2H])c([2H])c([2H])c([2H])c2[Si](c2c([2H])c([2H])c([2H])c([2H])c2[2H])(c2c([2H])c([2H])c([2H])c([2H])c2[2H])c2c([2H])c([2H])c([2H])c([2H])c21] | R174 |
| 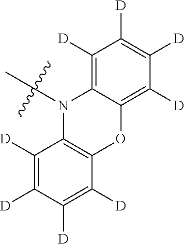 [SMILES: *N1c2c([2H])c([2H])c([2H])c([2H])c2Oc2c([2H])c([2H])c([2H])c([2H])c21] | R175 |
| 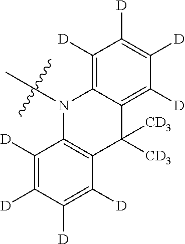 [SMILES: *N1c2c([2H])c([2H])c([2H])c([2H])c2C(C([2H])([2H])[2H])(C([2H])([2H])[2H])c2c([2H])c([2H])c([2H])c([2H])c21] | R176 |
| 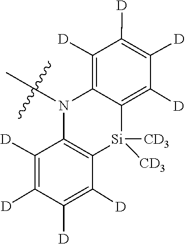 [SMILES: *N1c2c([2H])c([2H])c([2H])c([2H])c2[Si](C([2H])([2H])[2H])(C([2H])([2H])[2H])c2c([2H])c([2H])c([2H])c([2H])c21] | R177 |
| 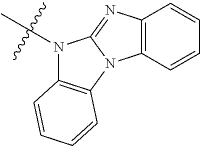 [SMILES: *n1c2ccccc2n2c3ccccc3nc12] | R178 |
| 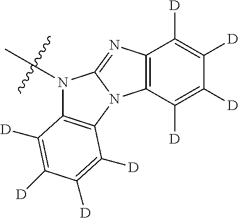 [SMILES: *n1c2c([2H])c([2H])c([2H])c([2H])c2n2c3c([2H])c([2H])c([2H])c([2H])c3nc12] | R179 |
| 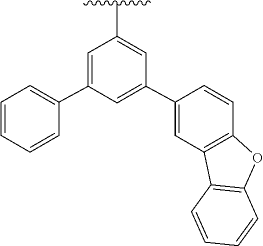 [SMILES: *c1cc(-c2ccccc2)cc(-c2ccc3oc4ccccc4c3c2)c1] | R180 |
| 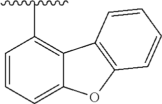 [SMILES: *c1cccc2oc3ccccc3c12] | R181 |
| 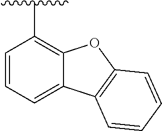 [SMILES: *c1cccc2c1oc1ccccc12] | R182 |
| 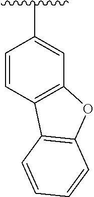 [SMILES: *c1ccc2c(c1)oc1ccccc12] | R183 |
| 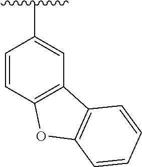 [SMILES: *c1ccc2oc3ccccc3c2c1] | R184 |
| 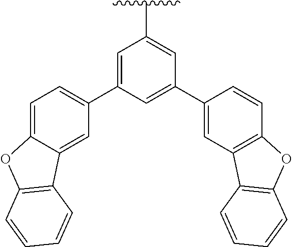 [SMILES: *c1cc(-c2ccc3oc4ccccc4c3c2)cc(-c2ccc3oc4ccccc4c3c2)c1] | R185 |
| 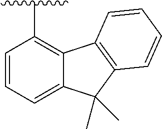 [SMILES: *c1cccc2c1-c1ccccc1C2(C)C] | R186 |
| 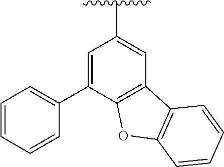 [SMILES: *c1cc(-c2ccccc2)c2oc3ccccc3c2c1] | R187 |
| 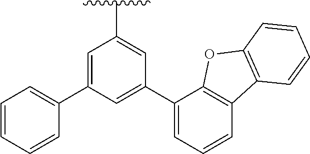 [SMILES: *c1cc(-c2ccccc2)cc(-c2cccc3c2oc2ccccc23)c1] | R188 |
| 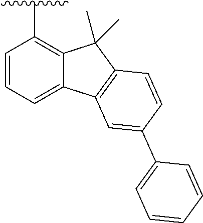 [SMILES: *c1cccc2c1C(C)(C)c1ccc(-c3ccccc3)cc1-2] | R189 |
| 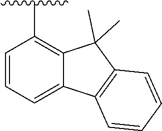 [SMILES: *c1cccc2c1C(C)(C)c1ccccc1-2] | R190 |
| 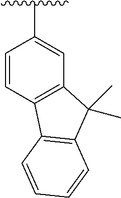 [SMILES: *c1ccc2c(c1)C(C)(C)c1ccccc1-2] | R191 |
| 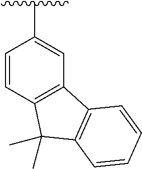 [SMILES: *c1ccc2c(c1)-c1ccccc1C2(C)C] | R192 |
| 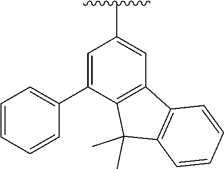 [SMILES: *c1cc(-c2ccccc2)c2c(c1)-c1ccccc1C2(C)C] | R193 |
| 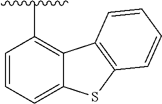 [SMILES: *c1cccc2sc3ccccc3c12] | R194 |
| 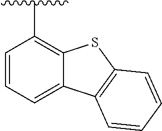 [SMILES: *c1cccc2c1sc1ccccc12] | R195 |
| 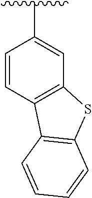 [SMILES: *c1ccc2c(c1)sc1ccccc12] | R196 |
| 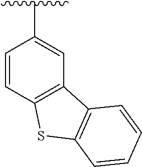 [SMILES: *c1ccc2sc3ccccc3c2c1] | R197 |
| 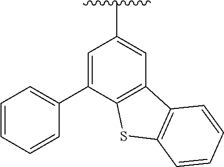 [SMILES: *c1cc(-c2ccccc2)c2sc3ccccc3c2c1] | R198 |
| 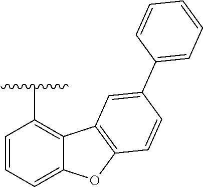 [SMILES: *c1cccc2oc3ccc(-c4ccccc4)cc3c12] | R199 |
| 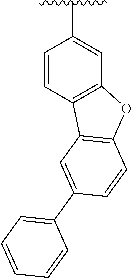 [SMILES: *c1ccc2c(c1)oc1ccc(-c3ccccc3)cc12] | R200 |
| 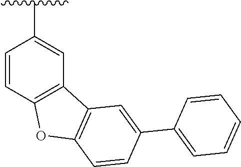 [SMILES: *c1ccc2oc3ccc(-c4ccccc4)cc3c2c1] | R201 |
| 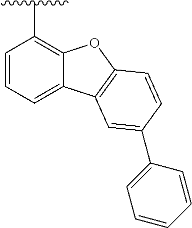 [SMILES: *c1cccc2c1oc1ccc(-c3ccccc3)cc12] | R202 |
| 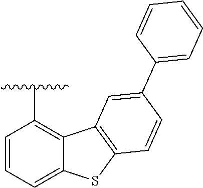 [SMILES: *c1cccc2sc3ccc(-c4ccccc4)cc3c12] | R203 |
| 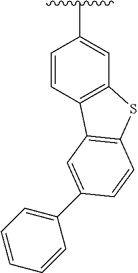 [SMILES: *c1ccc2c(c1)sc1ccc(-c3ccccc3)cc12] | R204 |
| 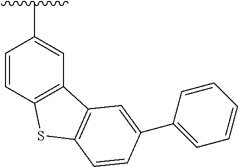 [SMILES: *c1ccc2sc3ccc(-c4ccccc4)cc3c2c1] | R205 |
| 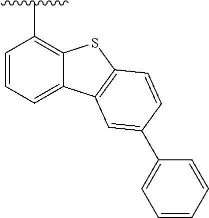 [SMILES: *c1cccc2c1sc1ccc(-c3ccccc3)cc12] | R206 |
| 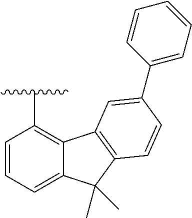 [SMILES: *c1cccc2c1-c1cc(-c3ccccc3)ccc1C2(C)C] | R207 |
| 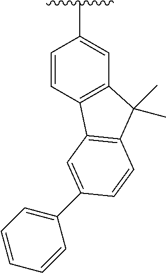 [SMILES: *c1ccc2c(c1)C(C)(C)c1ccc(-c3ccccc3)cc1-2] | R208 |
| 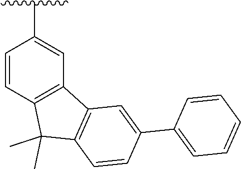 [SMILES: *c1ccc2c(c1)-c1cc(-c3ccccc3)ccc1C2(C)C] | R209 |
| 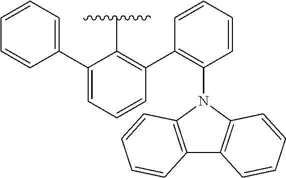 [SMILES: *c1c(-c2ccccc2)cccc1-c1ccccc1-n1c2ccccc2c2ccccc21] | R210 |
| 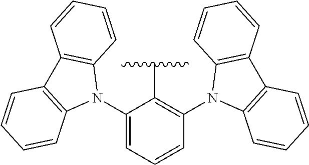 [SMILES: *c1c(-n2c3ccccc3c3ccccc32)cccc1-n1c2ccccc2c2ccccc21] | R211 |
| 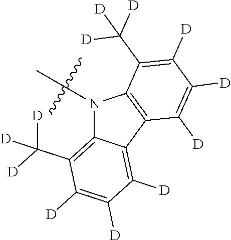 [SMILES: *n1c2c(C([2H])([2H])[2H])c([2H])c([2H])c([2H])c2c2c([2H])c([2H])c([2H])c(C([2H])([2H])[2H])c21] | R212 |
| 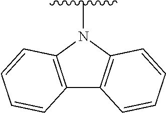 [SMILES: *n1c2ccccc2c2ccccc21] | R213 |
| 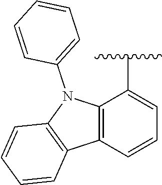 [SMILES: *c1cccc2c3ccccc3n(-c3ccccc3)c12] | R214 |
| 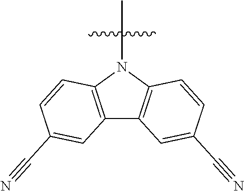 [SMILES: *n1c2ccc(C#N)cc2c2cc(C#N)ccc21] | R215 |
| 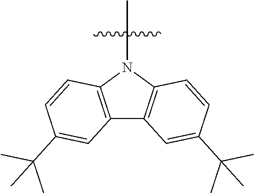 [SMILES: *n1c2ccc(C(C)(C)C)cc2c2cc(C(C)(C)C)ccc21] | R216 |
| 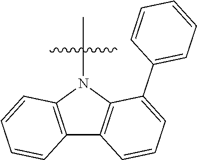 [SMILES: *n1c2ccccc2c2cccc(-c3ccccc3)c21] | R217 |
| 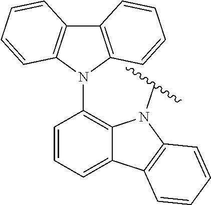 [SMILES: *n1c2ccccc2c2cccc(-n3c4ccccc4c4ccccc43)c21] | R218 |
| 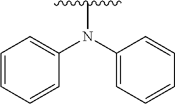 [SMILES: *N(c1ccccc1)c1ccccc1] | R219 |
| 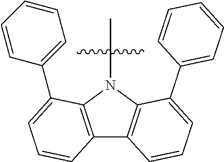 [SMILES: *n1c2c(-c3ccccc3)cccc2c2cccc(-c3ccccc3)c21] | R220 |
| 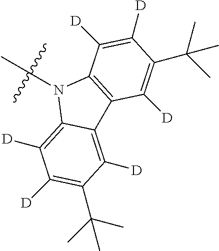 [SMILES: *n1c2c([2H])c([2H])c(C(C)(C)C)c([2H])c2c2c([2H])c(C(C)(C)C)c([2H])c([2H])c21] | R221 |
| 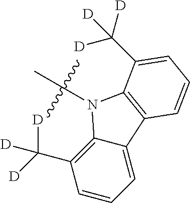 [SMILES: *n1c2c(C([2H])([2H])[2H])cccc2c2cccc(C([2H])([2H])[2H])c21] | R222 |
| 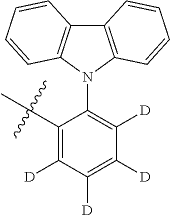 [SMILES: *c1c([2H])c([2H])c([2H])c([2H])c1-n1c2ccccc2c2ccccc21] | R223 |
| 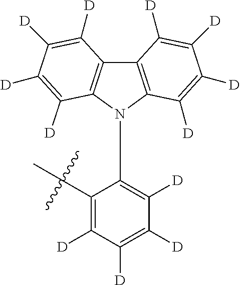 [SMILES: *c1c([2H])c([2H])c([2H])c([2H])c1Cn1c2c([2H])c([2H])c([2H])c([2H])c2c2c([2H])c([2H])c([2H])c([2H])c21] | R224 |
| 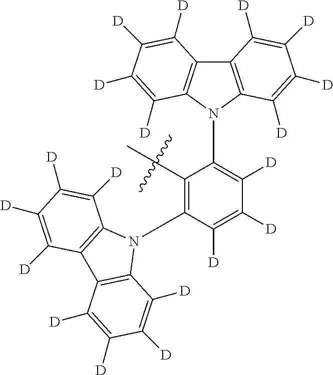 [SMILES: *c1c(-n2c3c([2H])c([2H])c([2H])c([2H])c3c3c([2H])c([2H])c([2H])c([2H])c32)c([2H])c([2H])c([2H])c1-n1c2c([2H])c([2H])c([2H])c([2H])c2c2c([2H])c([2H])c([2H])c([2H])c21] | R225 |
| 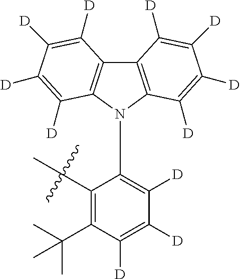 [SMILES: *c1c(-n2c3c([2H])c([2H])c([2H])c([2H])c3c3c([2H])c([2H])c([2H])c([2H])c32)c([2H])c([2H])c([2H])c1C(C)(C)C] | R226 |
| 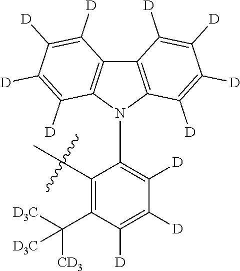 [SMILES: *c1c(-n2c3c([2H])c([2H])c([2H])c([2H])c3c3c([2H])c([2H])c([2H])c([2H])c32)c([2H])c([2H])c([2H])c1C(C([2H])([2H])[2H])(C([2H])([2H])[2H])C([2H])([2H])[2H]] | R227 |
| 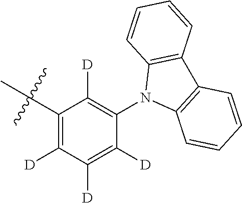 [SMILES: *c1c([2H])c([2H])c([2H])c(-n2c3ccccc3c3ccccc32)c1[2H]] | R228 |
| 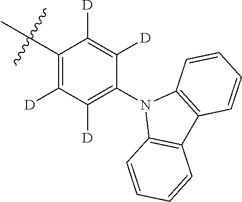 [SMILES: *c1c([2H])c([2H])c(-n2c3ccccc3c3ccccc32)c([2H])c1[2H]] | R229 |
| 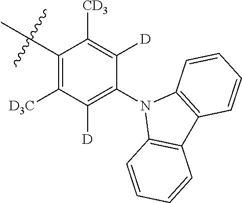 [SMILES: *c1c(C([2H])([2H])[2H])c([2H])c(-n2c3ccccc3c3ccccc32)c([2H])c1C([2H])([2H])[2H]] | R230 |
| 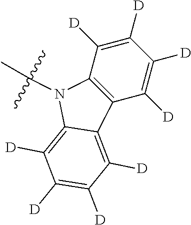 [SMILES: *n1c2c([2H])c([2H])c([2H])c([2H])c2c2c([2H])c([2H])c([2H])c([2H])c21] | R231 |
| 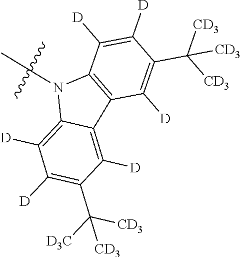 [SMILES: *n1c2c([2H])c([2H])c(C(C([2H])([2H])[2H])(C([2H])([2H])[2H])C([2H])([2H])[2H])c([2H])c2c2c([2H])c(C(C([2H])([2H])[2H])(C([2H])([2H])[2H])C([2H])([2H])[2H])c([2H])c([2H])c21] | R232 |
| 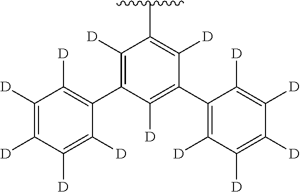 [SMILES: *c1c([2H])c(-c2c([2H])c([2H])c([2H])c([2H])c2[2H])c([2H])c(-c2c([2H])c([2H])c([2H])c([2H])c2[2H])c1[2H]] | R233 |
| 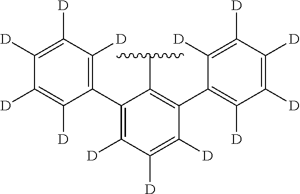 [SMILES: *c1c(-c2c([2H])c([2H])c([2H])c([2H])c2[2H])c([2H])c([2H])c([2H])c1-c1c([2H])c([2H])c([2H])c([2H])c1[2H]] | R234 |
| 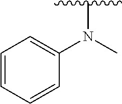 [SMILES: *N(C)c1ccccc1] | R235 |
| 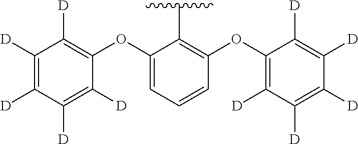 [SMILES: *c1c(Oc2c([2H])c([2H])c([2H])c([2H])c2[2H])cccc1Oc1c([2H])c([2H])c([2H])c([2H])c1[2H]] | R236 |
| 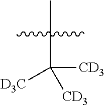 [SMILES: *C(C([2H])([2H])[2H])(C([2H])([2H])[2H])C([2H])([2H])[2H]] | R237 |
| 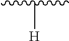 [SMILES: *[H]] | R238 |
| 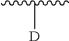 [SMILES: *[2H]] | R239 |
| 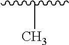 | R240 |
| 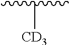 [SMILES: *C([2H])([2H])[2H]] | R241 |
| 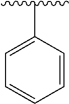 [SMILES: *c1ccccc1] | R242 |
| 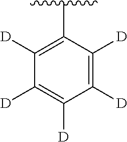 [SMILES: *c1c([2H])c([2H])c([2H])c([2H])c1[2H]] | R243 |
| 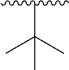 [SMILES: *C(C)(C)C] | R244 |
| 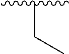 [SMILES: *CC] | R245 |
| 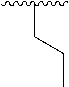 [SMILES: *CCC] | R246 |
| 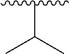 [SMILES: *C(C)C] | R247 |
| 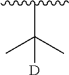 [SMILES: *C([2H])(C)C] | R248 |
| 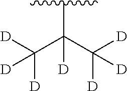 [SMILES: *C([2H])(C([2H])([2H])[2H])C([2H])([2H])[2H]] | R249 |
| 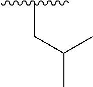 [SMILES: *CC(C)C] | R250 |
| 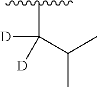 [SMILES: *C([2H])([2H])C(C)C] | R251 |
| 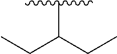 [SMILES: *C(CC)CC] | R252 |
| 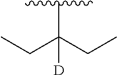 [SMILES: *C([2H])(CC)CC] | R253 |
| 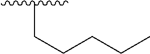 [SMILES: *CCCCC] | R254 |
| 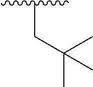 [SMILES: *CC(C)(C)C] | R255 |
| 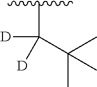 [SMILES: *C([2H])([2H])C(C)(C)C] | R256 |
| 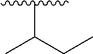 [SMILES: *C(C)CC] | R257 |
| 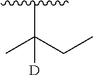 [SMILES: *C([2H])(C)CC] | R258 |
| 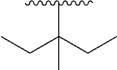 [SMILES: *C(C)(CC)CC] | R259 |
| 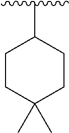 [SMILES: *C1CCC(C)(C)CC1] | R260 |
| 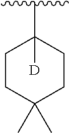 | R261 |
| 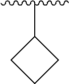 [SMILES: *C1CCC1] | R262 |
| 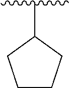 [SMILES: *C1CCCC1] | R263 |
| 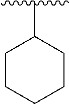 [SMILES: *C1CCCCC1] | R264 |
| 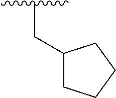 [SMILES: *CC1CCCC1] | R265 |
| 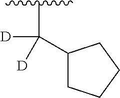 [SMILES: *C([2H])([2H])C1CCCC1] | R266 |
| 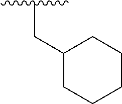 [SMILES: *CC1CCCCC1] | R267 |
| 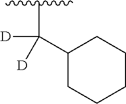 [SMILES: *C([2H])([2H])C1CCCCC1] | R268 |
| 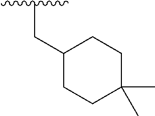 [SMILES: *CC1CCC(C)(C)CC1] | R269 |
| 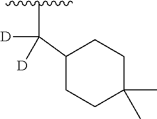 [SMILES: *C([2H])([2H])C1CCC(C)(C)CC1] | R270 |
| 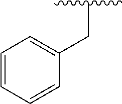 [SMILES: *Cc1ccccc1] | R271 |
| 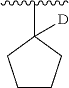 [SMILES: *C1([2H])CCCC1] | R272 |
| 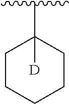 | R273 |
| 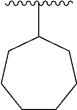 [SMILES: *C1CCCCCC1] | R274 |
| 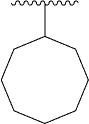 [SMILES: *C1CCCCCCC1] | R275 |
| 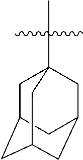 [SMILES: *C12CC3CC(CC(C3)C1)C2] | R276 |
| 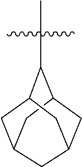 [SMILES: *C1C2CC3CC(C2)CC1C3] | R277 |
| 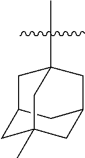 [SMILES: *C12CC3CC(C1)CC(C)(C3)C2] | R278 |
| 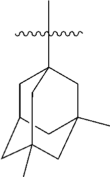 [SMILES: *C12CC3CC(C)(C1)CC(C)(C3)C2] | R279 |
| 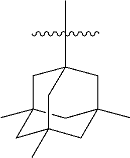 | R280 |
| 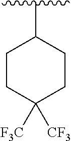 [SMILES: *C1CCC(C(F)(F)F)(C(F)(F)F)CC1] | R281 |
| 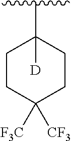 | R282 |
| 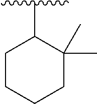 [SMILES: *C1CCCCC1(C)C] | R283 |
| 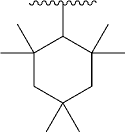 | R284 |
| 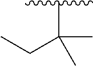 [SMILES: *C(C)(C)CC] | R285 |
| 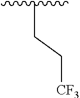 [SMILES: *CCC(F)(F)F] | R286 |
| 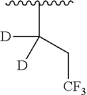 [SMILES: *C([2H])([2H])CC(F)(F)F] | R287 |
| 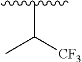 [SMILES: *C(C)C(F)(F)F] | R288 |
| 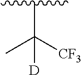 [SMILES: *C([2H])(C)C(F)(F)F] | R289 |
| 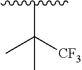 [SMILES: *C(C)(C)C(F)(F)F] | R290 |
| 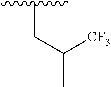 [SMILES: *CC(C)C(F)(F)F] | R291 |
| 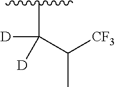 [SMILES: *C([2H])([2H])C(C)C(F)(F)F] | R292 |
| 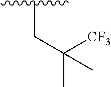 [SMILES: *CC(C)(C)C(F)(F)F] | R293 |
| 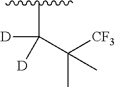 [SMILES: *C([2H])([2H])C(C)(C)C(F)(F)F] | R294 |
| 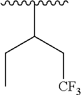 [SMILES: *C(CC)CC(F)(F)F] | R295 |
| 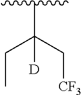 [SMILES: *C([2H])(CC)CC(F)(F)F] | R296 |
| 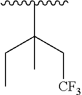 [SMILES: *C(C)(CC)CC(F)(F)F] | R297 |
| 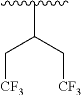 [SMILES: *C(CC(F)(F)F)CC(F)(F)F] | R298 |
| 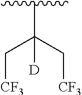 [SMILES: *C([2H])(CC(F)(F)F)CC(F)(F)F] | R299 |
| 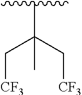 [SMILES: *C(C)(CC(F)(F)F)CC(F)(F)F] | R300 |
| 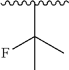 [SMILES: *C(C)(C)F] | R301 |
| 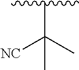 [SMILES: *C(C)(C)C#N] | R302 |
| 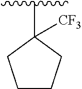 [SMILES: *C1(C(F)(F)F)CCCC1] | R303 |
| 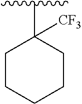 [SMILES: *C1(C(F)(F)F)CCCCC1] | R304 |
| 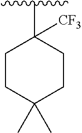 [SMILES: *C1(C(F)(F)F)CCC(C)(C)CC1] | R305 |
| 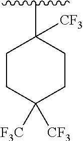 [SMILES: *C1(C(F)(F)F)CCC(C(F)(F)F)(C(F)(F)F)CC1] | R306 |
| 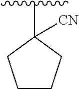 [SMILES: *C1(C#N)CCCC1] | R307 |
| 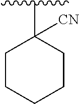 [SMILES: *C1(C#N)CCCCC1] | R308 |
| 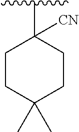 [SMILES: *C1(C#N)CCC(C)(C)CC1] | R309 |
| 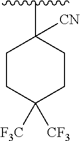 [SMILES: *C1(C#N)CCC(C(F)(F)F)(C(F)(F)F)CC1] | R310 |
| 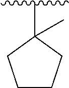 [SMILES: *C1(C)CCCC1] | R311 |
| 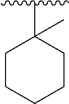 [SMILES: *C1(C)CCCCC1] | R312 |
| 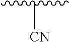 [SMILES: *C#N] | R313 |
| 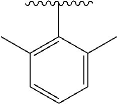 | R314 |
| 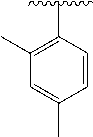 [SMILES: *c1ccc(C)cc1C] | R315 |
| 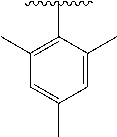 | R316 |
| 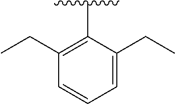 [SMILES: *c1c(CC)cccc1CC] | R317 |
| 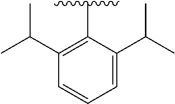 [SMILES: *c1c(C(C)C)cccc1C(C)C] | R318 |
| 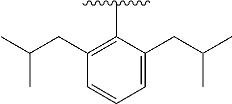 [SMILES: *c1c(CC(C)C)cccc1CC(C)C] | R319 |
| 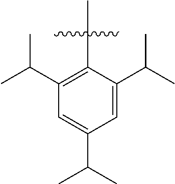 [SMILES: *c1c(C(C)C)cc(C(C)C)cc1C(C)C] | R320 |
| 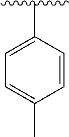 [SMILES: *c1ccc(C)cc1] | R321 |
| 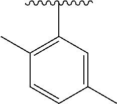 | R322 |
| 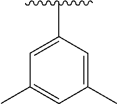 | R323 |
| 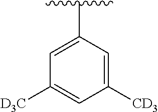 [SMILES: *c1cc(C([2H])([2H])[2H])cc(C([2H])([2H])[2H])c1] | R324 |
| 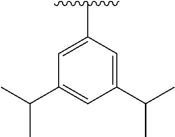 [SMILES: *c1cc(C(C)C)cc(C(C)C)c1] | R325 |
| 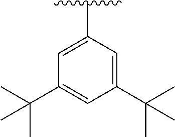 [SMILES: *c1cc(C(C)(C)C)cc(C(C)(C)C)c1] | R326 |
| 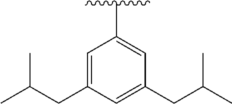 [SMILES: *c1cc(CC(C)C)cc(CC(C)C)c1] | R327 |
| 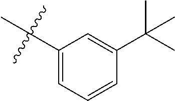 [SMILES: *c1cccc(C(C)(C)C)c1] | R328 |
| 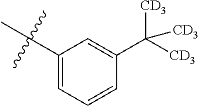 [SMILES: *c1cccc(C(C([2H])([2H])[2H])(C([2H])([2H])[2H])C([2H])([2H])[2H])c1] | R329 |
| 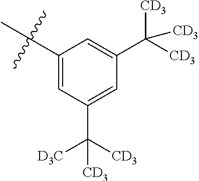 [SMILES: *c1cc(C(C([2H])([2H])[2H])(C([2H])([2H])[2H])C([2H])([2H])[2H])cc(C(C([2H])([2H])[2H])(C([2H])([2H])[2H])C([2H])([2H])[2H])c1] | R330 |
| 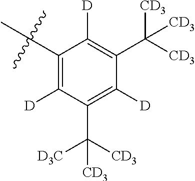 [SMILES: *c1c([2H])c(C(C([2H])([2H])[2H])(C([2H])([2H])[2H])C([2H])([2H])[2H])c([2H])c(C(C([2H])([2H])[2H])(C([2H])([2H])[2H])C([2H])([2H])[2H])c1[2H]] | R331 |
| 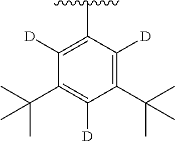 [SMILES: *c1c([2H])c(C(C)(C)C)c([2H])c(C(C)(C)C)c1[2H]] | R332 |
| 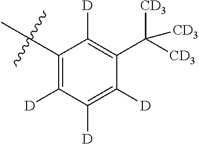 [SMILES: *c1c([2H])c([2H])c([2H])c(C(C([2H])([2H])[2H])(C([2H])([2H])[2H])C([2H])([2H])[2H])c1[2H]] | R333 |
| 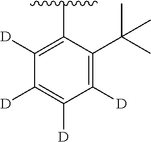 [SMILES: *c1c([2H])c([2H])c([2H])c([2H])c1C(C)(C)C] | R334 |
| 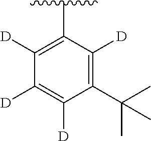 [SMILES: *c1c([2H])c([2H])c([2H])c(C(C)(C)C)c1[2H]] | R335 |
| 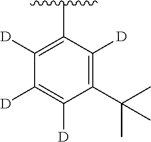 [SMILES: *c1c([2H])c([2H])c([2H])c(C(C)(C)C)c1[2H]] | R336 |
| 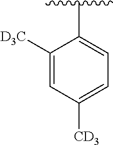 [SMILES: *c1ccc(C([2H])([2H])[2H])cc1C([2H])([2H])[2H]] | R337 |
| 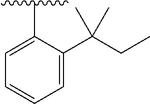 [SMILES: *c1ccccc1C(C)(C)CC] | R338 |
| 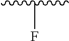 [SMILES: *F] | R339 |
| 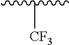 [SMILES: *C(F)(F)F] | R340 |
| 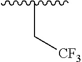 [SMILES: *CC(F)(F)F] | R341 |
| 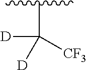 [SMILES: *C([2H])([2H])C(F)(F)F] | R342 |
| 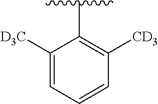 [SMILES: *c1c(C([2H])([2H])[2H])cccc1C([2H])([2H])[2H]] | R343 |
| 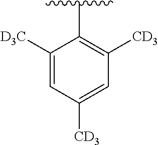 [SMILES: *c1c(C([2H])([2H])[2H])cc(C([2H])([2H])[2H])cc1C([2H])([2H])[2H]] | R344 |
| 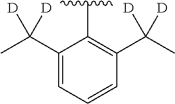 [SMILES: *c1c(C([2H])([2H])C)cccc1C([2H])([2H])C] | R345 |
| 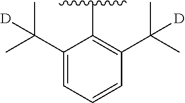 [SMILES: *c1c(C([2H])(C)C)cccc1C([2H])(C)C] | R346 |
| 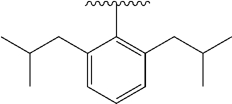 [SMILES: *c1c(CC(C)C)cccc1CC(C)C] | R347 |
| 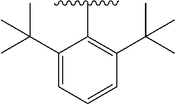 [SMILES: *c1c(C(C)(C)C)cccc1C(C)(C)C] | R348 |
| 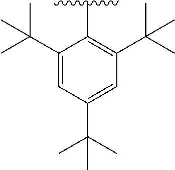 [SMILES: *c1c(C(C)(C)C)cc(C(C)(C)C)cc1C(C)(C)C] | R349 |
| 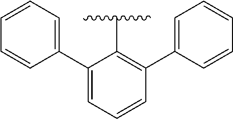 [SMILES: *c1c(-c2ccccc2)cccc1-c1ccccc1] | R350 |
| 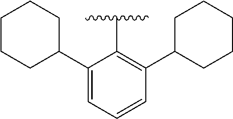 [SMILES: *c1c(C2CCCCC2)cccc1C1CCCCC1] | R351 |
| 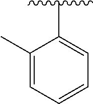 [SMILES: *c1ccccc1C] | R352 |
| 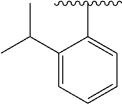 [SMILES: *c1ccccc1C(C)C] | R353 |
| 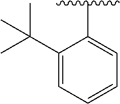 [SMILES: *c1ccccc1C(C)(C)C] | R354 |
| 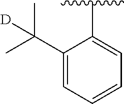 [SMILES: *c1ccccc1C([2H])(C)C] | R355 |
| 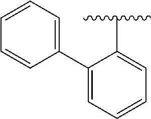 [SMILES: *c1ccccc1-c1ccccc1] | R356 |
| 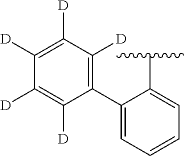 [SMILES: *c1ccccc1-c1c([2H])c([2H])c([2H])c([2H])c1[2H]] | R357 |
| 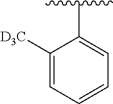 [SMILES: *c1ccccc1C([2H])([2H])[2H]] | R358 |
| 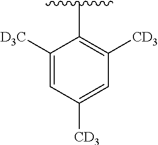 [SMILES: *c1c(C([2H])([2H])[2H])cc(C([2H])([2H])[2H])cc1C([2H])([2H])[2H]] | R359 |
| 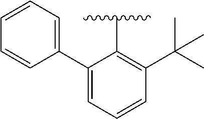 [SMILES: *c1c(-c2ccccc2)cccc1C(C)(C)C] | R360 |
| 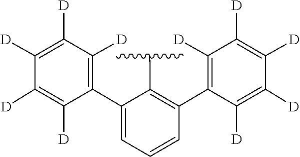 [SMILES: *c1c(-c2c([2H])c([2H])c([2H])c([2H])c2[2H])cccc1-c1c([2H])c([2H])c([2H])c([2H])c1[2H]] | R361 |
| 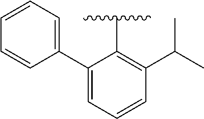 [SMILES: *c1c(-c2ccccc2)cccc1C(C)C] | R362 |
| 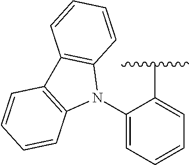 [SMILES: *c1ccccc1-n1c2ccccc2c2ccccc21] | R363 |
| 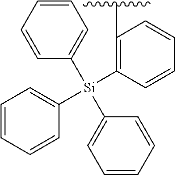 [SMILES: *c1ccccc1[Si](c1ccccc1)(c1ccccc1)c1ccccc1] | R364 |
| 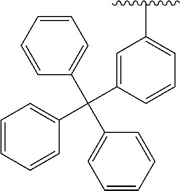 [SMILES: *c1cccc(C(c2ccccc2)(c2ccccc2)c2ccccc2)c1] | R365 |
| 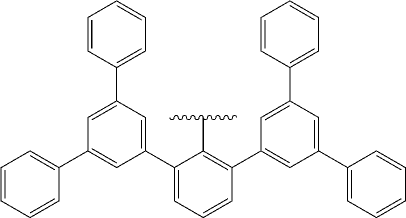 [SMILES: *c1c(-c2cc(-c3ccccc3)cc(-c3ccccc3)c2)cccc1-c1cc(-c2ccccc2)cc(-c2ccccc2)c1] | R366 |
| 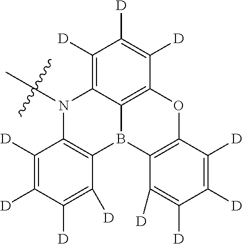 [SMILES: *N1c2c([2H])c([2H])c([2H])c([2H])c2B2c3c([2H])c([2H])c([2H])c([2H])c3Oc3c([2H])c([2H])c([2H])c1c32] | R367 |
| 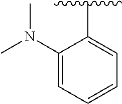 [SMILES: *c1ccccc1N(C)C] | R368 |
| 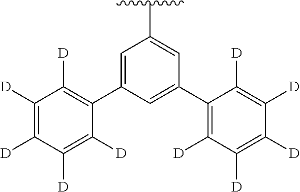 [SMILES: *c1cc(-c2c([2H])c([2H])c([2H])c([2H])c2[2H])cc(-c2c([2H])c([2H])c([2H])c([2H])c2[2H])c1] | R369 |
| 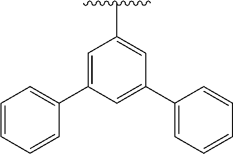 [SMILES: *c1cc(-c2ccccc2)cc(-c2ccccc2)c1] | R370 |
| 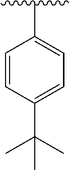 [SMILES: *c1ccc(C(C)(C)C)cc1] | R371 |
| 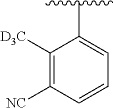 [SMILES: *c1cccc(C#N)c1C([2H])([2H])[2H]] | R372 |
| 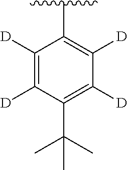 [SMILES: *c1c([2H])c([2H])c(C(C)(C)C)c([2H])c1[2H]] | R373 |
| 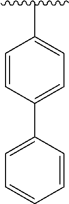 [SMILES: *c1ccc(-c2ccccc2)cc1] | R374 |
| 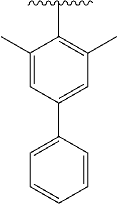 | R375 |
| 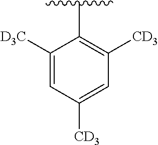 [SMILES: *c1c(C([2H])([2H])[2H])cc(C([2H])([2H])[2H])cc1C([2H])([2H])[2H]] | R376 |
| 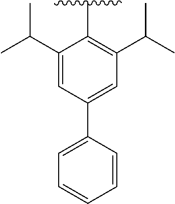 [SMILES: *c1c(C(C)C)cc(-c2ccccc2)cc1C(C)C] | R377 |
| 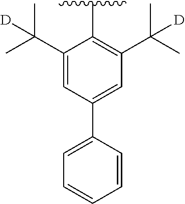 [SMILES: *c1c(C([2H])(C)C)cc(-c2ccccc2)cc1C([2H])(C)C] | R378 |
| 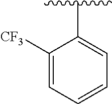 [SMILES: *c1ccccc1C(F)(F)F] | R379 |
| 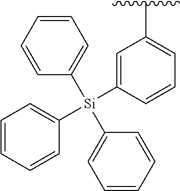 [SMILES: *c1cccc([Si](c2ccccc2)(c2ccccc2)c2ccccc2)c1] | R380 |
| 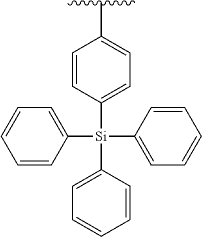 [SMILES: *c1ccc([Si](c2ccccc2)(c2ccccc2)c2ccccc2)cc1] | R381 |
| 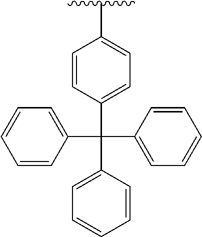 [SMILES: *c1ccc(C(c2ccccc2)(c2ccccc2)c2ccccc2)cc1] | R382 |
| 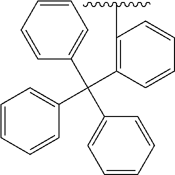 [SMILES: *c1ccccc1C(c1ccccc1)(c1ccccc1)c1ccccc1] | R383 |
| 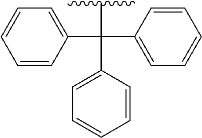 [SMILES: *C(c1ccccc1)(c1ccccc1)c1ccccc1] | R384 |
| 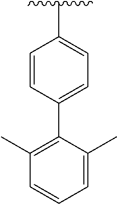 | R385 |
| 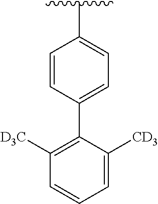 [SMILES: *c1ccc(-c2c(C([2H])([2H])[2H])cccc2C([2H])([2H])[2H])cc1] | R386 |
| 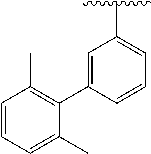 [SMILES: *c1cccc(-c2c(C)cccc2C)c1] | R387 |
| 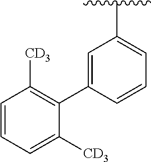 [SMILES: *c1cccc(-c2c(C([2H])([2H])[2H])cccc2C([2H])([2H])[2H])c1] | R388 |
| 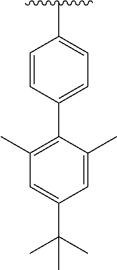 | R389 |
| 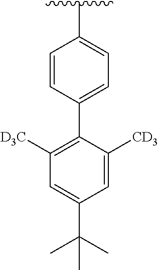 [SMILES: *c1ccc(-c2c(C([2H])([2H])[2H])cc(C(C)(C)C)cc2C([2H])([2H])[2H])cc1] | R390 |
| 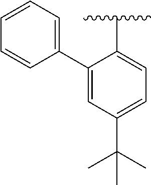 [SMILES: *c1ccc(C(C)(C)C)cc1-c1ccccc1] | R391 |
| 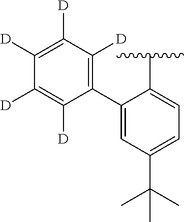 [SMILES: *c1ccc(C(C)(C)C)cc1-c1c([2H])c([2H])c([2H])c([2H])c1[2H]] | R392 |
| 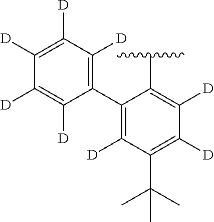 [SMILES: *c1c([2H])c([2H])c(C(C)(C)C)c([2H])c1-c1c([2H])c([2H])c([2H])c([2H])c1[2H]] | R393 |
| 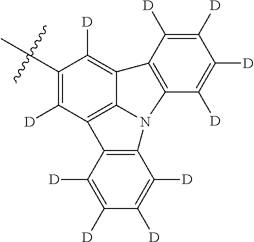 [SMILES: *c1c([2H])c2c3c([2H])c([2H])c([2H])c([2H])c3n3c4c([2H])c([2H])c([2H])c([2H])c4c(c1[2H])c23] | R394 |
| 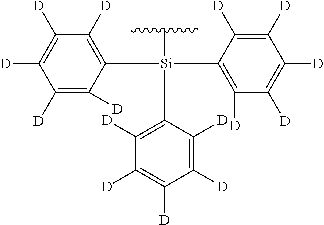 [SMILES: *[Si](c1c([2H])c([2H])c([2H])c([2H])c1[2H])(c1c([2H])c([2H])c([2H])c([2H])c1[2H])c1c([2H])c([2H])c([2H])c([2H])c1[2H]] | R395 |
| 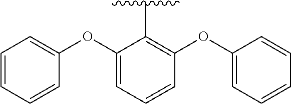 [SMILES: *c1c(Oc2ccccc2)cccc1Oc1ccccc1] | R396 |
| 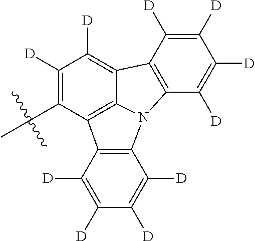 [SMILES: *c1c([2H])c([2H])c2c3c([2H])c([2H])c([2H])c([2H])c3n3c4c([2H])c([2H])c([2H])c([2H])c4c1c23] | R397 |
| 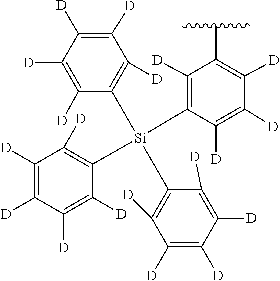 [SMILES: *c1c([2H])c([2H])c([2H])c([Si](c2c([2H])c([2H])c([2H])c([2H])c2[2H])(c2c([2H])c([2H])c([2H])c([2H])c2[2H])c2c([2H])c([2H])c([2H])c([2H])c2[2H])c1[2H]] | R398 |
| 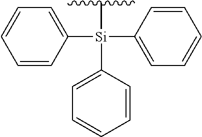 [SMILES: *[Si](c1ccccc1)(c1ccccc1)c1ccccc1] | R399 |
| 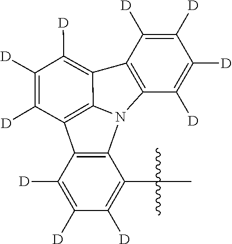 [SMILES: *c1c([2H])c([2H])c([2H])c2c3c([2H])c([2H])c([2H])c4c5c([2H])c([2H])c([2H])c([2H])c5n(c12)c34] | R400 |
| 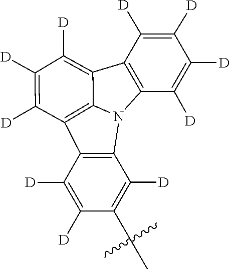 [SMILES: *c1c([2H])c([2H])c2c3c([2H])c([2H])c([2H])c4c5c([2H])c([2H])c([2H])c([2H])c5n(c2c1[2H])c34] | R401 |
| 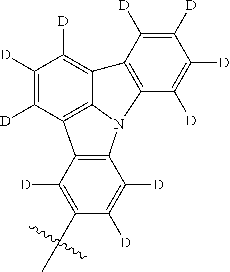 [SMILES: *c1c([2H])c([2H])c2c(c1[2H])c1c([2H])c([2H])c([2H])c3c4c([2H])c([2H])c([2H])c([2H])c4n2c13] | R402 |
| 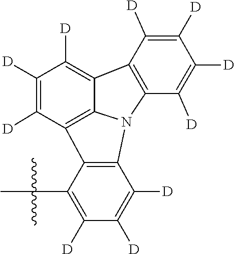 [SMILES: *c1c([2H])c([2H])c([2H])c2c1c1c([2H])c([2H])c([2H])c3c4c([2H])c([2H])c([2H])c([2H])c4n2c13] | R403 |
| 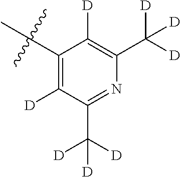 [SMILES: *c1c([2H])c(C([2H])([2H])[2H])nc(C([2H])([2H])[2H])c1[2H]] | R404 |
| 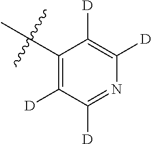 [SMILES: *c1c([2H])c([2H])nc([2H])c1[2H]] | R405 |
| 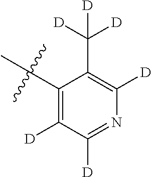 [SMILES: *c1c([2H])c([2H])nc([2H])c1C([2H])([2H])[2H]] | R406 |
| 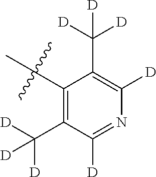 [SMILES: *c1c(C([2H])([2H])[2H])c([2H])nc([2H])c1C([2H])([2H])[2H]] | R407 |
| 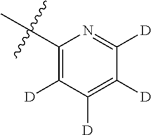 [SMILES: *c1nc([2H])c([2H])c([2H])c1[2H]] | R408 |
| 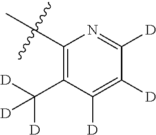 [SMILES: *c1nc([2H])c([2H])c([2H])c1C([2H])([2H])[2H]] | R409 |
| 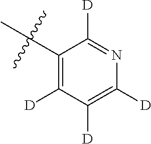 [SMILES: *c1c([2H])nc([2H])c([2H])c1[2H]] | R410 |
| 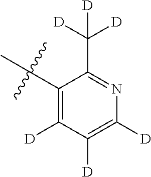 [SMILES: *c1c(C([2H])([2H])[2H])nc([2H])c([2H])c1[2H]] | R411 |
| 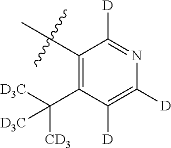 [SMILES: *c1c([2H])nc([2H])c([2H])c1C(C([2H])([2H])[2H])(C([2H])([2H])[2H])C([2H])([2H])[2H]] | R412 |
| 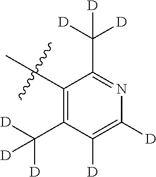 [SMILES: *c1c(C([2H])([2H])[2H])nc([2H])c([2H])c1C([2H])([2H])[2H]] | R413 |
| 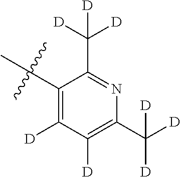 [SMILES: *c1c(C([2H])([2H])[2H])nc(C([2H])([2H])[2H])c([2H])c1[2H]] | R414 |
| 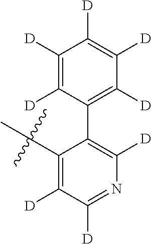 [SMILES: *c1c([2H])c([2H])nc([2H])c1-c1c([2H])c([2H])c([2H])c([2H])c1[2H]] | R415 |
| 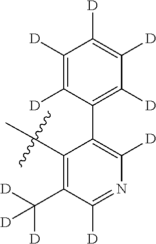 [SMILES: *c1c(-c2c([2H])c([2H])c([2H])c([2H])c2[2H])c([2H])nc([2H])c1C([2H])([2H])[2H]] | R416 |
| 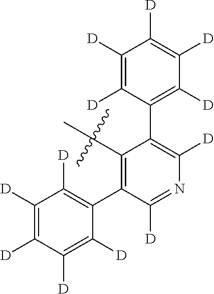 [SMILES: *c1c(-c2c([2H])c([2H])c([2H])c([2H])c2[2H])c([2H])nc([2H])c1-c1c([2H])c([2H])c([2H])c([2H])c1[2H]] | R417 |
| 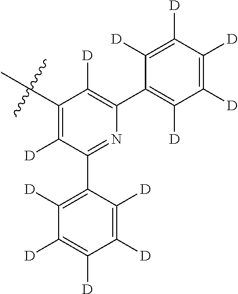 [SMILES: *c1c([2H])c(-c2c([2H])c([2H])c([2H])c([2H])c2[2H])nc(-c2c([2H])c([2H])c([2H])c([2H])c2[2H])c1[2H]] | R418 |
| 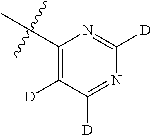 [SMILES: *c1nc([2H])nc([2H])c1[2H]] | R419 |
| 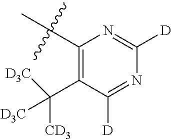 [SMILES: *c1nc([2H])nc([2H])c1C(C([2H])([2H])[2H])(C([2H])([2H])[2H])C([2H])([2H])[2H]] | R420 |
| 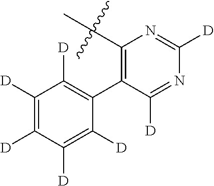 [SMILES: *c1nc([2H])nc([2H])c1-c1c([2H])c([2H])c([2H])c([2H])c1[2H]] | R421 |
| 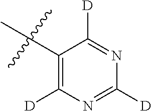 [SMILES: *c1c([2H])nc([2H])nc1[2H]] | R422 |
| 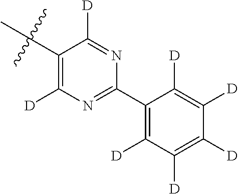 [SMILES: *c1c([2H])nc(-c2c([2H])c([2H])c([2H])c([2H])c2[2H])nc1[2H]] | R423 |
| 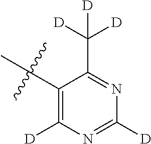 [SMILES: *c1c([2H])nc([2H])nc1C([2H])([2H])[2H]] | R424 |
| 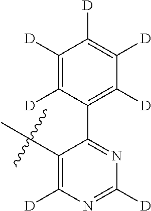 [SMILES: *c1c([2H])nc([2H])nc1-c1c([2H])c([2H])c([2H])c([2H])c1[2H]] | R425 |
| 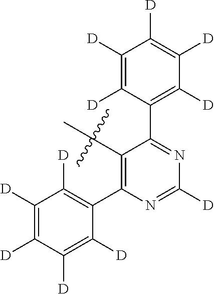 | R426 |
| 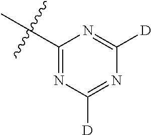 [SMILES: *c1nc([2H])nc([2H])n1] | R427 |
| 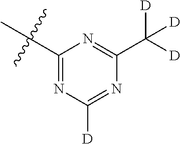 [SMILES: *c1nc([2H])nc(C([2H])([2H])[2H])n1] | R428 |
| 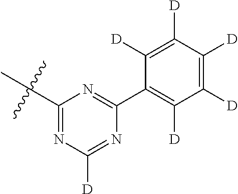 [SMILES: *c1nc([2H])nc(-c2c([2H])c([2H])c([2H])c([2H])c2[2H])n1] | R429 |
| 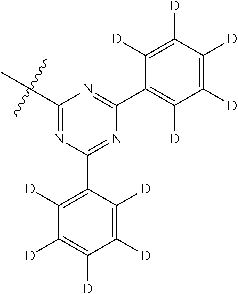 [SMILES: *c1nc(-c2c([2H])c([2H])c([2H])c([2H])c2[2H])nc(-c2c([2H])c([2H])c([2H])c([2H])c2[2H])n1] | R430 |
| 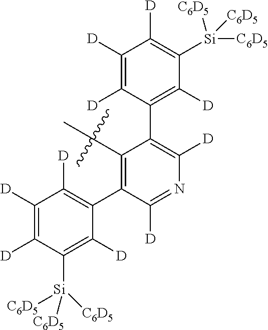 | R431 |
| 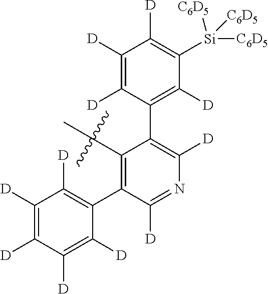 | R432 |
| 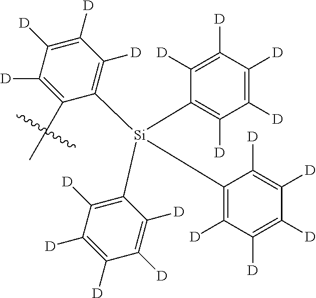 [SMILES: *c1c([2H])c([2H])c([2H])c([2H])c1[Si](c1c([2H])c([2H])c([2H])c([2H])c1[2H])(c1c([2H])c([2H])c([2H])c([2H])c1[2H])c1c([2H])c([2H])c([2H])c([2H])c1[2H]] | R433 |
| 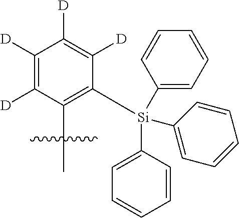 [SMILES: *c1c([2H])c([2H])c([2H])c([2H])c1[Si](c1ccccc1)(c1ccccc1)c1ccccc1] | R434 |
| 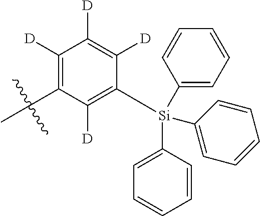 [SMILES: *c1c([2H])c([2H])c([2H])c([Si](c2ccccc2)(c2ccccc2)c2ccccc2)c1[2H]] | R435 |
| 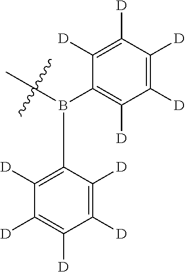 [SMILES: *B(c1c([2H])c([2H])c([2H])c([2H])c1[2H])c1c([2H])c([2H])c([2H])c([2H])c1[2H]] | R436 |
| 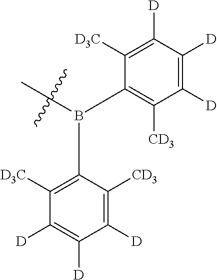 [SMILES: *B(c1c(C([2H])([2H])[2H])c([2H])c([2H])c([2H])c1C([2H])([2H])[2H])c1c(C([2H])([2H])[2H])c([2H])c([2H])c([2H])c1C([2H])([2H])[2H]] | R437 |
| 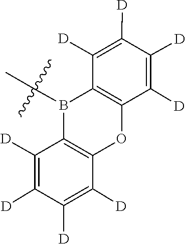 [SMILES: *B1c2c([2H])c([2H])c([2H])c([2H])c2Oc2c([2H])c([2H])c([2H])c([2H])c21] | R438 |
| 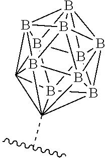 | R439 |
| 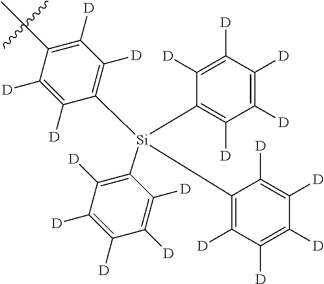 [SMILES: *c1c([2H])c([2H])c([Si](c2c([2H])c([2H])c([2H])c([2H])c2[2H])(c2c([2H])c([2H])c([2H])c([2H])c2[2H])c2c([2H])c([2H])c([2H])c([2H])c2[2H])c([2H])c1[2H]] | R440 |
| 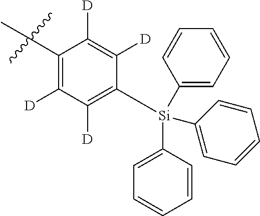 [SMILES: *c1c([2H])c([2H])c([Si](c2ccccc2)(c2ccccc2)c2ccccc2)c([2H])c1[2H]] | R441 |
| 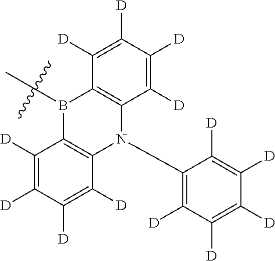 [SMILES: *B1c2c([2H])c([2H])c([2H])c([2H])c2N(c2c([2H])c([2H])c([2H])c([2H])c2[2H])c2c([2H])c([2H])c([2H])c([2H])c21] | R442 |
| 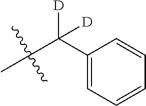 [SMILES: *C([2H])([2H])c1ccccc1] | R443 |
| 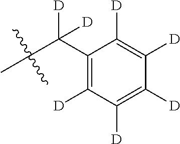 [SMILES: *C([2H])([2H])c1c([2H])c([2H])c([2H])c([2H])c1[2H]] | R444 |
| 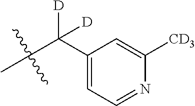 [SMILES: *C([2H])([2H])c1ccnc(C([2H])([2H])[2H])c1] | R445 |
| 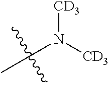 [SMILES: *N(C([2H])([2H])[2H])C([2H])([2H])[2H]] | R446 |
| 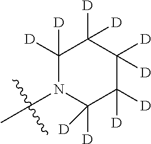 [SMILES: *N1C([2H])([2H])C([2H])([2H])C([2H])([2H])C([2H])([2H])C1([2H])[2H]] | R447 |
| 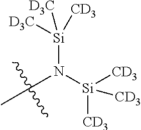 [SMILES: *N([Si](C([2H])([2H])[2H])(C([2H])([2H])[2H])C([2H])([2H])[2H])[Si](C([2H])([2H])[2H])(C([2H])([2H])[2H])C([2H])([2H])[2H]] | R448 |
| 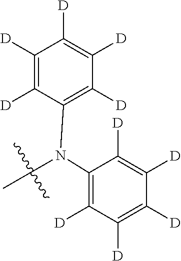 [SMILES: *N(c1c([2H])c([2H])c([2H])c([2H])c1[2H])c1c([2H])c([2H])c([2H])c([2H])c1[2H]] | R449 |
| 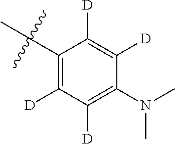 [SMILES: *c1c([2H])c([2H])c(N(C)C)c([2H])c1[2H]] | R450 |
| 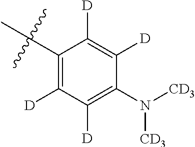 [SMILES: *c1c([2H])c([2H])c(N(C([2H])([2H])[2H])C([2H])([2H])[2H])c([2H])c1[2H]] | R451 |
| 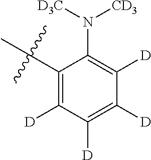 [SMILES: *c1c([2H])c([2H])c([2H])c([2H])c1N(C([2H])([2H])[2H])C([2H])([2H])[2H]] | R452 |
| 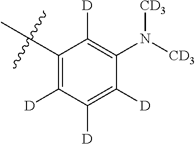 [SMILES: *c1c([2H])c([2H])c([2H])c(N(C([2H])([2H])[2H])C([2H])([2H])[2H])c1[2H]] | R453 |
| 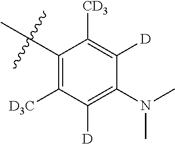 [SMILES: *c1c(C([2H])([2H])[2H])c([2H])c(N(C)C)c([2H])c1C([2H])([2H])[2H]] | R454 |
| 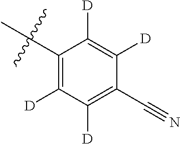 [SMILES: *c1c([2H])c([2H])c(C#N)c([2H])c1[2H]] | R455 |
| 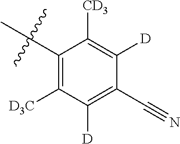 [SMILES: *c1c(C([2H])([2H])[2H])c([2H])c(C#N)c([2H])c1C([2H])([2H])[2H]] | R456 |
| 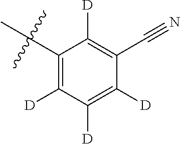 [SMILES: *c1c([2H])c([2H])c([2H])c(C#N)c1[2H]] | R457 |
| 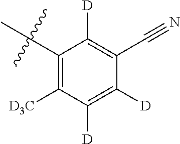 [SMILES: *c1c([2H])c(C#N)c([2H])c([2H])c1C([2H])([2H])[2H]] | R458 |
| 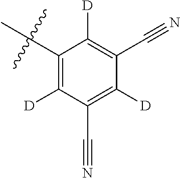 [SMILES: *c1c([2H])c(C#N)c([2H])c(C#N)c1[2H]] | R459 |
| 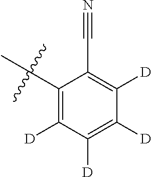 [SMILES: *c1c([2H])c([2H])c([2H])c([2H])c1C#N] | R460 |
| 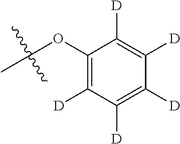 [SMILES: *Oc1c([2H])c([2H])c([2H])c([2H])c1[2H]] | R461 |
| 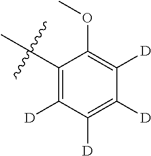 [SMILES: *c1c([2H])c([2H])c([2H])c([2H])c1OC] | R462 |
| 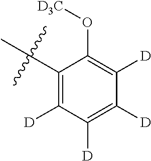 [SMILES: *c1c([2H])c([2H])c([2H])c([2H])c1OC([2H])([2H])[2H]] | R463 |
| 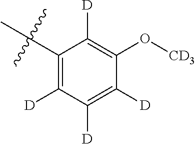 [SMILES: *c1c([2H])c([2H])c([2H])c(OC([2H])([2H])[2H])c1[2H]] | R464 |
| 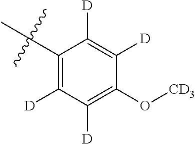 [SMILES: *c1c([2H])c([2H])c(OC([2H])([2H])[2H])c([2H])c1[2H]] | R465 |
| 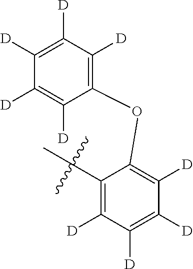 [SMILES: *c1c([2H])c([2H])c([2H])c([2H])c1Oc1c([2H])c([2H])c([2H])c([2H])c1[2H]] | R466 |
| 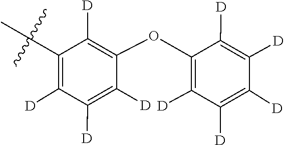 [SMILES: *c1c([2H])c([2H])c([2H])c(Oc2c([2H])c([2H])c([2H])c([2H])c2[2H])c1[2H]] | R467 |
| 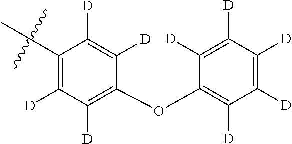 [SMILES: *c1c([2H])c([2H])c(Oc2c([2H])c([2H])c([2H])c([2H])c2[2H])c([2H])c1[2H]] | R468 |

In some embodiments, Rr is selected from R1 to R232, and the rest of Rj, Rk, and Rl is each independently an integer from R1 to R468. However, it should be understood that the same is intended for each of Rj, Rk, and Rl. For example, only one of Rj, Rk, Rl, or Rq must be selected from R178 to R468, and the rest can each independently be selected from R1 to R468. In other embodiments, only one of Rj, Rk, Rl, or Rr can be selected from R1 to R232. In still other embodiments, two of Rj, Rk, Rl, or Rr can be independently selected from R1 to R232. As a result, all the above identified embodiments/combinations are intended to be specifically covered. In other words, in alternate embodiments, the structure of Formula I (e.g., R1 to R232) can be required in one of positions Rj, Rk, or Rl, while Rr can be selected from R1 to R468.

By the same token, even though Rs is selected from R1 to R209, and the rest of Rj, Rk, and Rl is each independently an integer from R1 to R468. However, it should be understood that the same is intended for each of Rj, Rk, and Rl. For example, only one of Rj, Rk, Rl, or Rs must be selected from R210 to R468, and the rest can each independently be selected from R1 to R468. In other embodiments, only one of Rj, Rk, Rl, or Rs can be selected from R1 to R209. In still other embodiments, two of Rj, Rk, Rl, or Rs can be independently selected from R1 to R209. As a result, all the above identified embodiments/combinations are intended to be specifically covered. In other words, in alternate embodiments, the structure of Formula I (e.g., R1 to R209) can be required in one of positions Rj, Rk, or Rl, while Rs can be selected from R1 to R468.

In some embodiments, the compound is selected from the group consisting of (LIST 4):

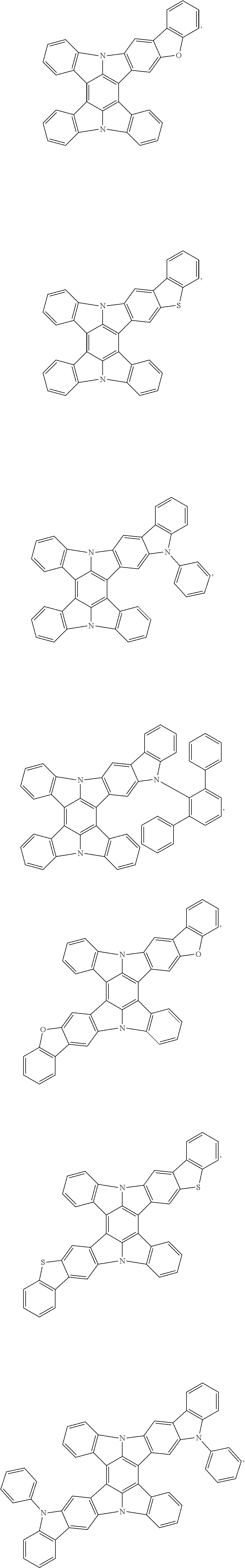

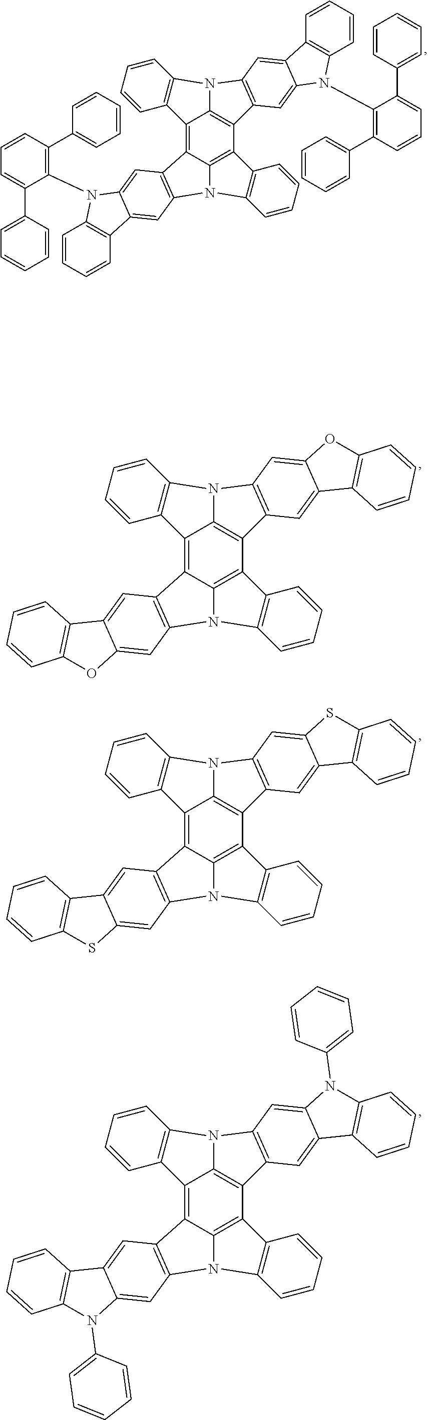 [SMILES: c1ccc(-c2cccc(-c3ccccc3)c2-n2c3ccccc3c3cc4c(cc32)c2c3c5ccccc5n5c6cc7c8ccccc8n(-c8c(-c9ccccc9)cccc8-c8ccccc8)c7cc6c(c6c7ccccc7n4c62)c35)cc1 ; c1ccc2c(c1)oc1cc3c(cc12)c1c2c4ccccc4n4c5cc6oc7ccccc7c6cc5c(c5c6ccccc6n3c51)c24 ; c1ccc2c(c1)sc1cc3c(cc12)c1c2c4ccccc4n4c5cc6sc7ccccc7c6cc5c(c5c6ccccc6n3c51)c24 ; c1ccc(-n2c3ccccc3c3cc4c5c6c7ccccc7n7c8cc9c(cc8c(c8c%10ccccc%10n(c4cc32)c85)c67)c2ccccc2n9-c2ccccc2)cc1]

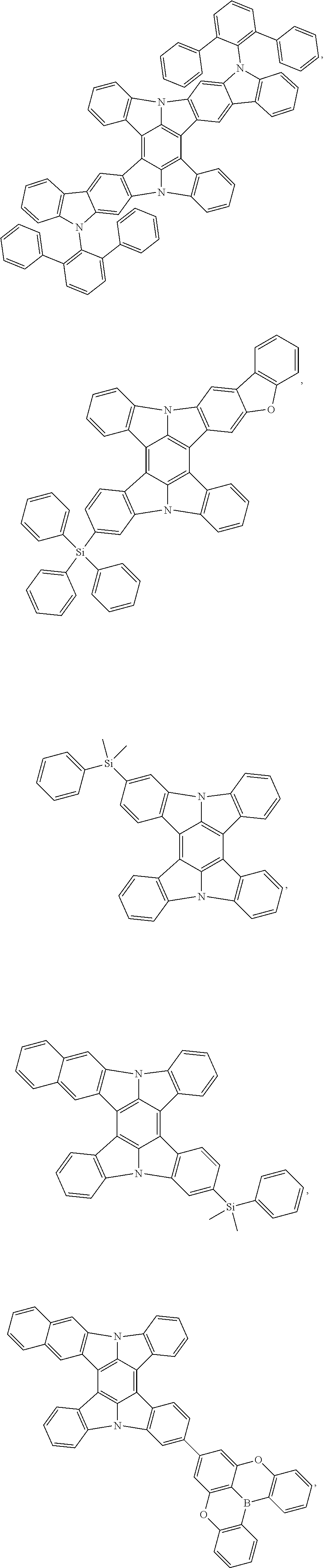

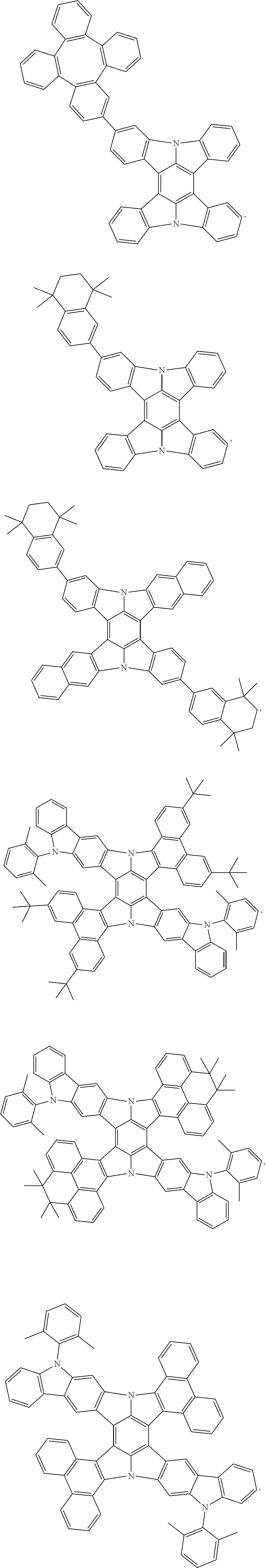

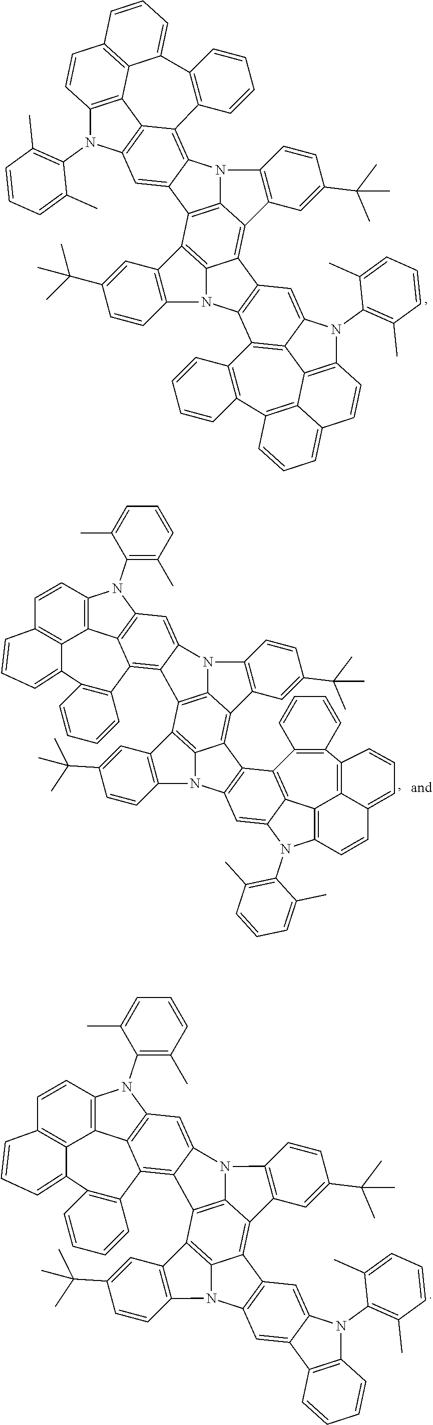 [SMILES: Cc1cccc(C)c1-n1c2ccc3cccc4c5ccccc5c5c(c2c34)c1cc1c2c3c4cc(C(C)(C)C)ccc4n4c6c(cc7c8c9c%10c(cccc%10c%10ccccc%10c86)ccc9n7-c6c(C)cccc6C)c(c6c7cc(C(C)(C)C)ccc7n(c15)c62)c34 ; Cc1cccc(C)c1-n1c2ccc3cccc4c5ccccc5c5c(c2c34)c1cc1c5c2c3c4cc(C(C)(C)C)ccc4n4c5cc6c7c(c8ccccc8c8cccc9ccc(c7c98)n6-c6c(C)cccc6C)c5c(c5c6cc(C(C)(C)C)ccc6n1c52)c34 ; Cc1cccc(C)c1-n1c2ccccc2c2cc3c(cc21)c1c2c4cc(C(C)(C)C)ccc4n4c5cc6c7c(c8ccccc8c8cccc9ccc(c7c98)n6-c6c(C)cccc6C)c5c(c5c6cc(C(C)(C)C)ccc6n3c15)c24]

In some embodiments of Formula I, at least one R^{Y1}, R^{Y2}, R^{Y3}, R^{Y4}, R^{Y5}, R^{A}, R^{B}, R^{C}, R^{D}, or R^{E} is partially or fully deuterated. In some embodiments, at least one R^{A} is partially or fully deuterated. In some embodiments, at least one R^{B} is partially or fully deuterated. In some embodiments, at least one R^{C} is partially or fully deuterated. In some embodiments, at least one R^{D} is partially or fully deuterated. In some embodiments, at least one R^{E} is partially or fully deuterated. In some embodiments, at least one R^{Y1} is partially or fully deuterated. In some embodiments, at least one R^{Y2} is partially or fully deuterated. In some embodiments, at least one R^{Y3} is partially or fully deuterated. In some embodiments, at least one R^{Y4} is partially or fully deuterated. In some embodiments, at least one R^{Y5} is partially or fully deuterated.

In some embodiments, at least one of R^{Y1}, R^{Y2}, R^{Y3}, R^{Y4}, R^{Y5}, R^{A}, R^{B}, R^{C}, R^{D}, or R^{E} is a substituent selected from the group consisting of the General Substituents defined herein. In some embodiments, at least one R^{A} is selected from the group consisting of the General Substituents defined herein. In some embodiments, at least one R^{B} is selected from the group consisting of the General Substituents defined herein. In some embodiments, at least one R^{C} is selected from the group consisting of the General Substituents defined herein. In some embodiments, at least one R^{D} is selected from the group consisting of the General Substituents defined herein. In some embodiments, at least one R^{E} is selected from the group consisting of the General Substituents defined herein. In some embodiments, at least one R^{Y1} is selected from the group consisting of the General Substituents defined herein. In some embodiments, at least one R^{Y2} is selected from the group consisting of the General Substituents defined herein. In some embodiments, at least one R^{Y3} is selected from the group consisting of the General Substituents defined herein. In some embodiments, at least one R^{Y4} is selected from the group consisting of the General Substituents defined herein. In some embodiments, at least one R^{Y5} is selected from the group consisting of the General Substituents defined herein.

In some embodiments, at least one of R^{Y1}, R^{Y2}, R^{Y3}, R^{Y4}, R^{Y5}, R^{A}, R^{B}, R^{C}, R^{D}, or R^{E} is a substituent selected from the group consisting of the Preferred General Substituents defined herein. In some embodiments, at least one R^{A} is selected from the group consisting of the Preferred General Substituents defined herein. In some embodiments, at least one R^{B} is selected from the group consisting of the Preferred General Substituents defined herein. In some embodiments, at least one R^{C} is selected from the group consisting of the Preferred General Substituents defined herein. In some embodiments, at least one R^{D} is selected from the group consisting of the Preferred General Substituents defined herein. In some embodiments, at least one R^{E} is selected from the group consisting of the Preferred General Substituents defined herein. In some embodiments, at least one R^{Y1} is selected from the group consisting of the Preferred General Substituents defined herein. In some embodiments, at least one R^{Y2} is selected from the group consisting of the Preferred General Substituents defined herein. In some embodiments, at least one R^{Y3} is selected from the group consisting of the Preferred General Substituents defined herein. In some embodiments, at least one R^{Y4} is selected from the group consisting of the Preferred General Substituents defined herein. In some embodiments, at least one R^{Y5} is selected from the group consisting of the Preferred General Substituents defined herein.

In some embodiments, the compound of Formula I described herein can be at least 30% deuterated, at least 40% deuterated, at least 50% deuterated, at least 60% deuterated, at least 70% deuterated, at least 80% deuterated, at least 90% deuterated, at least 95% deuterated, at least 99% deuterated, or 100% deuterated. As used herein, percent deuteration has its ordinary meaning and includes the percent of all possible hydrogen atoms (e.g., positions that are hydrogen or deuterium) that are occupied by deuterium atoms. In some embodiments, one or more hole transporting moieties are partially or fully deuterated. In some embodiments, one or more electron transporting moieties are partially or fully deuterated. In some embodiments, one or more fused ring systems are partially or fully deuterated. In some embodiments, one or more non-fused rings are partially or fully deuterated. In some embodiments, one or more rings or fused rings containing one or more heteroatoms are partially or fully deuterated. In some embodiments, one or more fused or non-fused phenyl rings are partially or fully deuterated. In some embodiments, one or more alkyl or cycloalkyl are partially or fully deuterated.

In yet another aspect of the present disclosure, a formulation that comprises the novel compound disclosed herein is described. The formulation can include one or more components selected from the group consisting of a solvent, an emitter, a host, a hole injection material, hole transport material, electron blocking material, hole blocking material, and an electron transport material, disclosed herein.

The present disclosure encompasses any chemical structure comprising the novel compound of the present disclosure, or a monovalent or polyvalent variant thereof. In other words, the inventive compound, or a monovalent or polyvalent variant thereof, can be a part of a larger chemical structure. Such chemical structure can be selected from the group consisting of a monomer, a polymer, a macromolecule, and a supramolecule (also known as supermolecule). As used herein, a “monovalent variant of a compound” refers to a moiety that is identical to the compound except that one hydrogen has been removed and replaced with a bond to the rest of the chemical structure. As used herein, a “polyvalent variant of a compound” refers to a moiety that is identical to the compound except that more than one hydrogen has been removed and replaced with a bond or bonds to the rest of the chemical structure. In the instance of a supramolecule, the inventive compound can also be incorporated into the supramolecule complex without covalent bonds. As used in this context, the description that a structure A comprises a moiety B means that the structure A includes the structure of moiety B not including the H or D atoms that can be attached to the moiety B. This is because at least one H or D on a given moiety structure has to be replaced to become a substituent so that the moiety B can be part of the structure A, and one or more of the H or D on a given moiety B structure can be further substituted once it becomes a part of structure A.

### C. The OLEDs and the Devices of the Present Disclosure

In another aspect, the present disclosure provides an organic light emitting device (OLED) comprising: an anode;
a cathode; and
an emissive region disposed between the anode and the cathode,
wherein the emissive region comprises a compound as described herein for the Compounds of the Present Disclosure.

In some embodiments, the present disclosure provides an organic light emitting device (OLED) comprising: an anode;
a cathode; and
an emissive region disposed between the anode and the cathode,
wherein the emissive region comprises a compound comprising a structure of Formula I

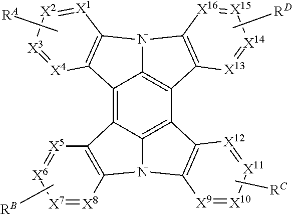

wherein each of X¹ to X¹⁶ is independently C or N;
each of R^{A}, R^{B}, R^{C}, and R^{D} independently represents mono to the maximum allowable substitutions, or no substitutions;
wherein any two substituents may be joined or fused to form a ring;
with the proviso that, if the compound comprises a structure of Formula II:

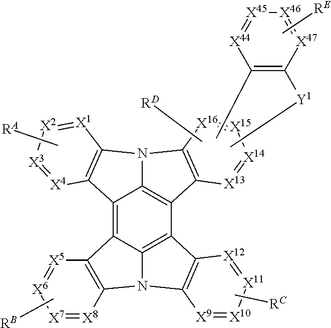

wherein each of X⁴⁴ to X⁴⁷ is independently C or N;
wherein Y¹ is selected from the group consisting of O, NR^{Y1}, and CMe₂;
wherein R^{E} represents mono to the maximum allowable substitutions, or no substitutions;
then
(i) when Y¹ is O or CMe₂, then X¹ is C, and
(ii) when Y¹ is NR^{Y1}, then R^{Y1} and R^{E} do not join to form a 5-membered ring;
with the proviso that, if no two of R^{A}, two of R^{B}, two of R^{C}, or two of R^{D} are joined to form a ring, then at least one of R^{A}, R^{B}, R^{C}, or R^{D} comprises a structure selected from the group consisting of alkyldiarylsilyl, dialkylarylsilyl, germyl, tetraphenylene, bicyclic alkyl, and an aromatic ring fused by a non-aromatic ring;
with the proviso that, if two of R^{A}, two of R^{B}, two of R^{C}, or two of R^{D} are joined to form a benzene ring, then at least one of R^{A}, R^{B}, R^{C}, or R^{D} comprises a structure selected from the group consisting of silyl, germyl, tetraphenylene, benzo[d]benzo[4,5]imidazo[1,2-a]imidazole (bimbim), bicyclic alkyl, an aromatic ring fused by a non-aromatic ring, Formula III, and Formula IV,

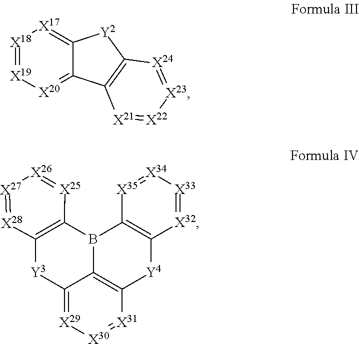

wherein each of X¹⁷ to X³⁵ is independently C or N, and
each Y², Y³, and Y⁴ is independently selected from the group consisting of O, S, Se, NR^{Y2}, BR^{Y2}, BR^{Y2}R^{Y3}, CR^{Y2}R^{Y3}, SiR^{Y2}R^{Y3}, GeR^{Y2}R^{Y3}, C═O, C═S, C═NR^{Y2}, C═CR^{Y2}R^{Y3}, SO, SO₂, or P(O)R^{Y2};
with the proviso that, if two of R^{A}, two of R^{B}, two of R^{C}, or two of R^{D} are joined to form an 5 or 6-membered cycloalkyl ring, then at least one of R^{A}, R^{B}, R^{C}, or R^{D} comprises a structure selected from the group consisting of alkyldiarylsilyl, dialkylarylsilyl, triarylsilane, substituted triphenylsilyl, germyl, tetraphenylene, benzo[d]benzo[4,5]imidazo[1,2-a]imidazole, bicyclic alkyl, an aromatic ring fused by a non-aromatic ring, and Formula V:

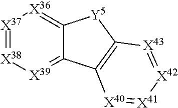

wherein each of X³⁶ to X⁴³ is independently C or N, and
wherein Y⁵ is selected from the group consisting of O, S, Se, BR⁴, BR^{Y4}R^{Y5}, CR^{Y4}R^{Y5}, SiR^{Y4}R^{Y5}, GeR^{Y4}R^{Y5}, C═O, C═S, C═NR^{Y4}, C═CR^{Y4}R^{Y5}, SO, SO₂, or P(O)R^{Y4};
wherein each of R^{Y1}, R^{Y2}, R^{Y3}, R^{Y4}, R^{Y5}, R^{A}, R^{B}, R^{C}, R^{D}, and R^{E} is independently hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof;
wherein the compound is not:

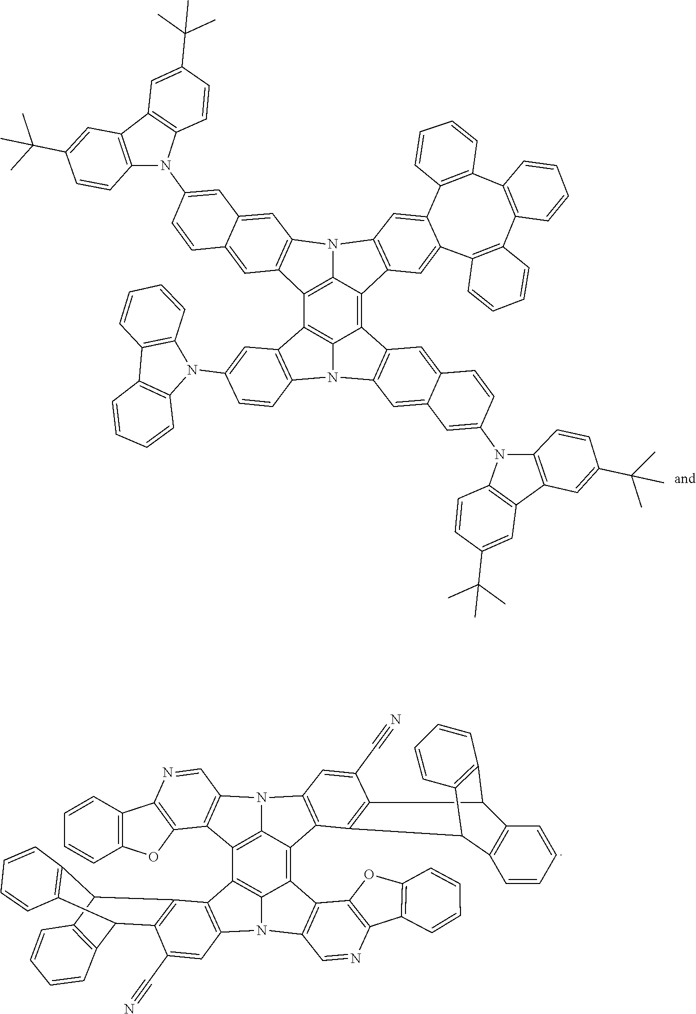 [SMILES: CC(C)(C)c1ccc2c(c1)c1cc(C(C)(C)C)ccc1n2-c1ccc2cc3c4c5c6cc7c(cc6n6c8cc9cc(-n%10c%11ccc(C(C)(C)C)cc%11c%11cc(C(C)(C)C)ccc%11%10)ccc9cc8c(c8c9cc(-n%10c%11ccccc%11c%11ccccc%11%10)ccc9n(c3cc2c1)c48)c56)-c1ccccc1-c1ccccc1-c1ccccc1-7 ; N#Cc1cc2c(c3c1C1c4ccccc4C3c3ccccc31)c1c3c4c5oc6ccccc6c5ncc4n4c5cc(C#N)c6c(c5c(c5c7c8oc9ccccc9c8ncc7n2c15)c34)C1c2ccccc2C6c2ccccc21]

In some embodiments, the compound of formula I is a host. If in the OLED of the present invention, the compound of formula I is a host, the OLED further contains at least one sensitizer.

In some embodiments, the compound of formula I is a fluorescent emitter. If in the OLED of the present invention, the compound of formula I is a fluorescent emitter, the OLED further contains at least one host.

In some embodiments, the compound of formula I can be used as a fluorescent emitter.

In some embodiments, the compound of formula I is a sensitizer compound.

In some embodiments, the emissive region does not comprise a metal complex.

In some embodiments, the emissive region further comprises a first host.

In some embodiments, the emissive region consists of the compound and the first host.

In some embodiments, the emissive region further comprises a second host.

In some embodiments, the first host comprises at least one anthracene.

In some embodiments, the first host has a S₁ energy (the first singlet energy) higher than the S₁ of any compound of the Present Disclosure as described herein.

In some embodiments, the first host has a T₁ energy (the first triplet energy) lower than the T₁ of any compound of the Present Disclosure as described herein.

In some embodiments, the first host is capable of P-type delayed fluorescence.

In some embodiments, the first host is fully or partially deuterated.

In some embodiments, the first host consists of aromatic hydrocarbon rings. This means that in such embodiments, the first host does not comprise heterocyclic rings.

In some embodiments, the second host is fully or partially deuterated.

In some embodiments, the second host consists of aromatic hydrocarbon rings.

In some embodiments, the first host has a LUMO level lower than the LUMO of any compound of v.

In some embodiments, the first host has a HOMO level lower than the HOMO of any compound of the Present Disclosure as described herein.

In some embodiments, the first host and the second host are capable of forming an exciplex.

In some embodiments, the compounds of the present inventive can be used as an acceptor.

In some embodiments, the emissive region further comprises a compound S1;
wherein the compound comprises the structure of Formula I is an acceptor (A1);
wherein the compound S1 is a sensitizer capable of energy transfer from at least one of its excited states to an excited state of the acceptor A1 at room temperature.

In some embodiments, S1 is one of the components of an exciplex.

In some embodiments, the exciplex is formed by two or more materials comprise an organometallic complex and a host.

In some embodiments, the organometallic complex comprises a phosphorescent material, a fluorescent material, or a thermally-activated delayed fluorescent material.

In some embodiments, the host comprises at least one chemical group selected from the group consisting of triphenylene, carbazole, indolocarbazole, dibenzothiophene, dibenzofuran, dibenzoselenophene, 5λ²-benzo[d]benzo[4,5]imidazo[3,2-a]imidazole, 5,9-dioxa-13b-boranaphtho[3,2,1-de]anthracene, azaborinine, oxaborinine, dihydroacridine, xanthene, dihydrobenzoazasiline, dibenzooxasiline, phenoxazine, phenoxathiine, phenothiazine, dihydrophenazine, fluorene, naphthalene, anthracene, phenanthrene, phenanthroline, benzoquinoline, quinoline, isoquinoline, quinazoline, pyrimidine, pyrazine, pyridine, triazine, boryl, silyl, aza-triphenylene, aza-carbazole, aza-indolocarbazole, aza-dibenzothiophene, aza-dibenzofuran, aza-dibenzoselenophene, aza-5λ²-benzo[d]benzo[4,5]imidazo[3,2-a]imidazole, and aza-(5,9-dioxa-13b-boranaphtho[3,2,1-de]anthracene).

In some embodiments, S1 and A1 are not covalently linked.

In some embodiments, S1 and A1 are covalently linked by a direct bond or an organic linker L1*.

In some embodiments, S1 and A1 are covalently linked by a direct bond.

In some embodiments, S1 and A1 are covalently linked by an organic linker L1* that comprises a structure selected from the group consisting of the structures of the following LIST 5:

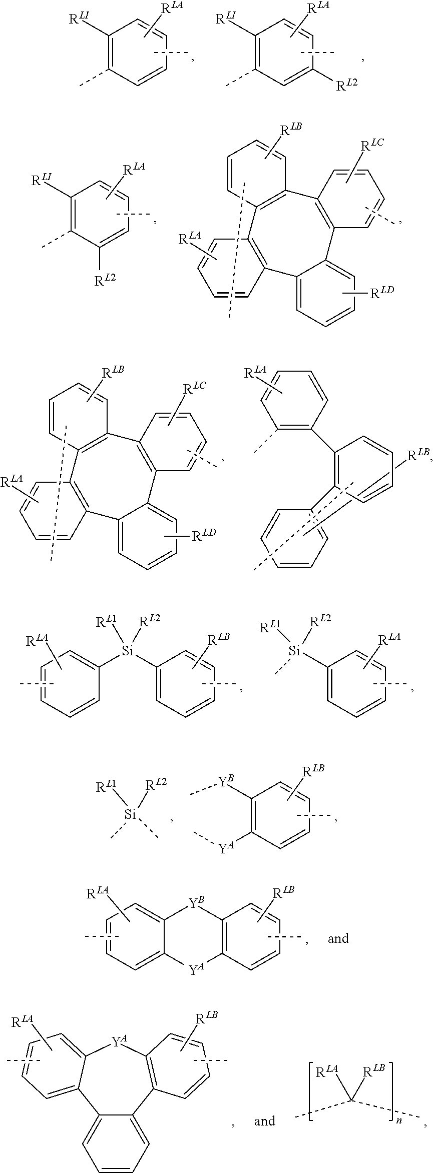

wherein:
n is an integer from 1 to 10;
each of YA and Y^{B} is independently selected from the group consisting of BR, NR, PR, O, S, Se, C═O, S═O, SO₂, BRR′, CRR′, SiRR′, and GeRR′;
each of R^{LA}, R^{LB}, R^{LC}, and R^{LD} independently represents from mono to maximum possible number of substitutions, or no substitutions;
each R, R′, R^{LA}, R^{LB}, R^{LC}, and R^{LD} is independently a hydrogen or a substituent selected from the group consisting of deuterium, halogen, alkyl, cycloalkyl, heteroalkyl, heterocycloalkyl, boryl, arylalkyl, alkoxy, aryloxy, amino, silyl, germyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof;
R^{L1} and R^{L2} are each independently selected from the group consisting of alkyl, cycloalkyl, heteroalkyl, heterocycloalkyl, arylalkyl, alkoxy, aryloxy, amino, silyl, germyl alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, boryl, deuterated variants thereof, and combinations thereof; and
two adjacent of R, R′, R^{L1}, R^{LA}, R^{LB}, R^{LC}, and R^{LD} are optionally joined or fused to form a ring.

In some embodiments, the sensitizer S1 and the acceptor A1 are covalently linked by an organic linker L1* that comprises a structure selected from the group consisting of the structures of the following LIST 6:

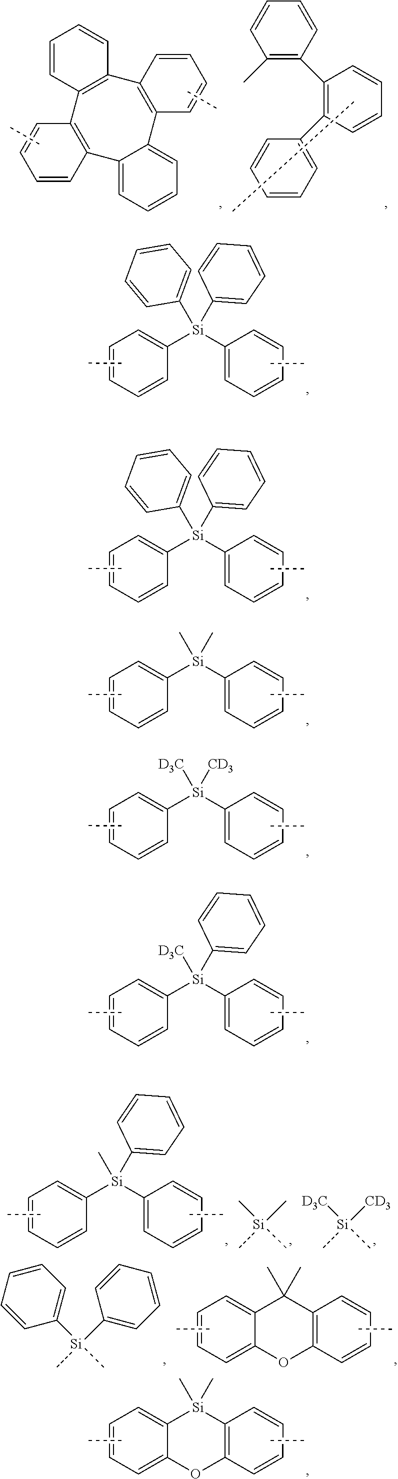

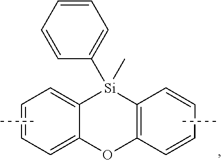

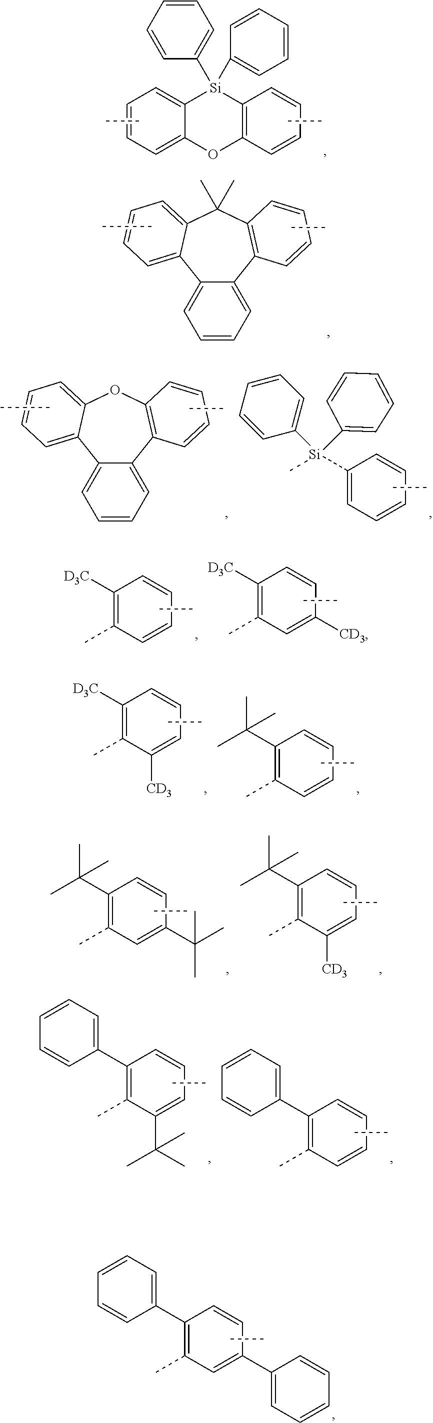

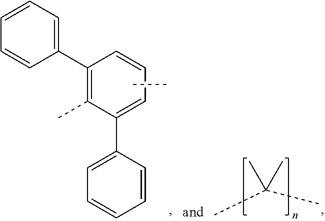

where n is an integer from 1 to 10; and fully or partially deuterated variants thereof.

In some embodiments, the compound S1 is capable of energy transfer from its excited state manifold to an excited state manifold of compound A1.

In some embodiments, the compound S1 has an emission peak from 400 nm to 480 nm.

In some embodiments, the compound S1 has an emission peak from 480 nm to 800 nm.

In some embodiments, the sensitizer S1 has an emission peak from 400 to 420 nm. In some embodiments, the sensitizer S1 has an emission peak from 420 to 440 nm. In some embodiments, the sensitizer S1 has an emission peak from 440 to 470 nm. In some embodiments, the sensitizer S1 has an emission peak from 470 to 500 nm. In some embodiments, the sensitizer S1 has an emission peak from 500 to 550 nm. In some embodiments, the sensitizer S1 has an emission peak from 600 to 670 nm. In some embodiments, the sensitizer S1 has an emission peak from 670 to 800.

In some embodiments, the compound A1 has a stokes shift of less than 100 nm.

In some embodiments, the compound A1 has a stokes shift of less than 80 nm.

In some embodiments, the compound A1 has a stokes shift of less than 60 nm.

In some embodiments, the compound A1 has a stokes shift of less than 40 nm.

In some embodiments, a spectral overlap integral of compound A1 and compound S1 is at least 10¹⁴ nm^{4*}L/cm*mol. A spectral overlap integral” is determined by multiplying the acceptor A1 extinction spectrum by the sensitizer S1 emission spectrum normalized with respect to area under the curve. The higher the spectral overlap, the better the FRET efficiency. The rate of Forster Resonance Energy Transfer (FRET) is proportional to the spectral overlap integral, therefore a high spectral overlap is desired to improve the FRET efficiency and reduce the exciton lifetime in the device. Increasing the spectral overlap can be achieved in several ways, for example, increasing the oscillator strength of acceptor A1, minimizing the distance between the sensitizer S1 emission peak and the acceptor A1 absorption peak, and narrowing the lineshape of the sensitizer S1 emission or the acceptor A1 absorption.

In some embodiments, a spectral overlap integral of compound A1 and compound S1 is at least 5×10¹⁴ nm^{4*}L/cm*mol.

In some embodiments, a spectral overlap integral of compound A1 and compound S1 is at least 10¹⁵ nm^{4*}L/cm*mol.

In some embodiments, the compound S1 is a phosphorescent material, a fluorescent material, or a thermally activated delayed fluorescent material.

In some embodiments, the compound S1 is a phosphorescent material.

In some embodiments, the compound S1 is an organometallic complex.

In some embodiments, the compound S1 is an organometallic Ir-complex.

In some embodiments, the compound S1 is an organometallic Pt-complex.

In some embodiments, the compound S1 is an organometallic Pd-complex.

In some embodiments, the compound S1 is an organometallic Cu-complex, Ag-complex, or Au-complex.

In some embodiments, the compound S1 has the formula of M(L^{1*})ₓ(L^{2*})_{y}(L^{3*})_{z};
wherein L^{1*}, L^{2*}, and L^{3*} can be the same or different;
wherein x is 1, 2, or 3;
wherein y is 0, 1, or 2;
wherein z is 0, 1, or 2;
wherein metal M is selected from the group consisting of Ir, Pt, Pd, Au, Ag, and Cu;
wherein x+y+z is the oxidation state of the metal M;
wherein L^{1*} is selected from the group consisting of the structures of the LIGAND LIST as defined herein;
wherein L^{2*} and L^{3*} are independently selected from the group consisting of

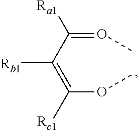

and the structures of LIGAND LIST; wherein:
T is selected from the group consisting of B, A1, Ga, and In;
K^{1′} is a direct bond or is selected from the group consisting of NRₑ, PRₑ, O, S, and Se;
each Y¹ to Y¹³ are independently selected from the group consisting of carbon and nitrogen;
Y′ is selected from the group consisting of BRₑ, NRₑ, PRₑ, O, S, Se, C═O, S═O, SO₂, CRₑR_{f}, SiRₑR_{f}, and GeRₑR_{f};
Rₑ and R_{f} can be fused or joined to form a ring;
each Rₐ, R_{b}, R_{c}, and R_{d} can independently represent from mono to the maximum possible number of substitutions, or no substitution;
each Rₐ₁, R_{b1}, R_{c1}, R_{d1}, Rₐ, R_{b}, R_{c}, R_{d}, Rₑ, and R_{f} is independently a hydrogen or a substituent selected from the group consisting of deuterium, halogen, alkyl, cycloalkyl, heteroalkyl, heterocycloalkyl, boryl, arylalkyl, alkoxy, aryloxy, amino, silyl, germyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof; and
wherein any two of Rₐ₁, R_{b1}, R_{c1}, R_{d1}, Rₐ, R_{b}, R_{c}, and R_{d} can be fused or joined to form a ring or form a multidentate ligand.

In some embodiments, the metal M is Ir, Pt, or Pd.

In some embodiments, the metal is Ir.

In some embodiments, the metal is Pt.

In some embodiments, the metal is Pd]

In some embodiments, the sensitizer S1 has a formula selected from the group consisting of the structures as defined in the SENSITIZER LIST as defined herein:

It should be understood that in the SENSITIZER LIST as defined herein for each of the compounds having Pt, Pt can be replaced by Pd, and those Pd derivatives are also intended to be specifically covered.

In some embodiments, L^{2*} is selected from the group consisting of L_{Bk}, wherein k is an integer from 1 to 621, and each of L_{B1} to L_{B621} has a structure defined in the following LIST 8:

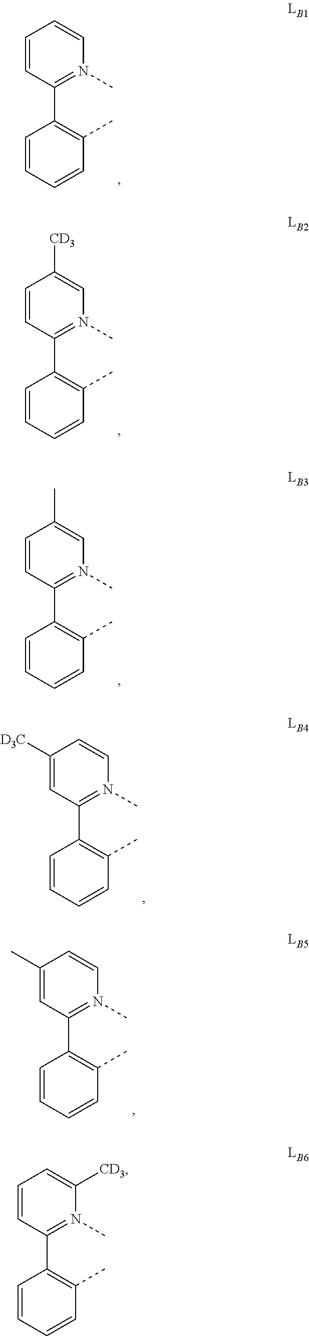

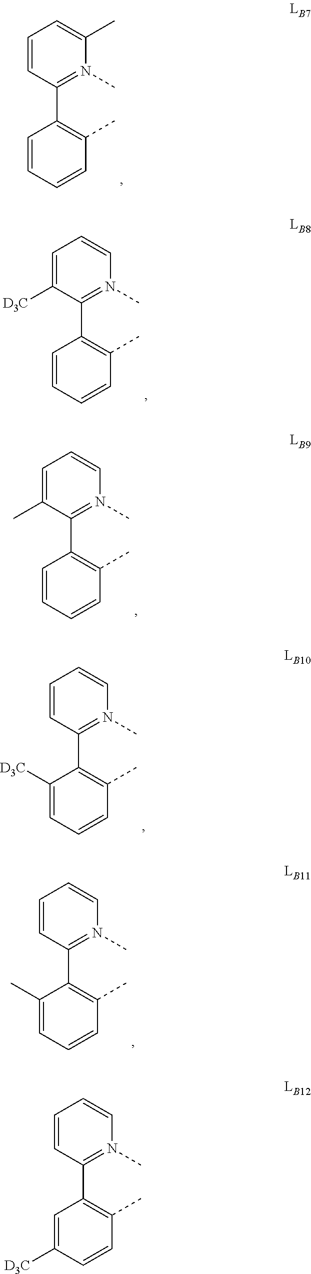

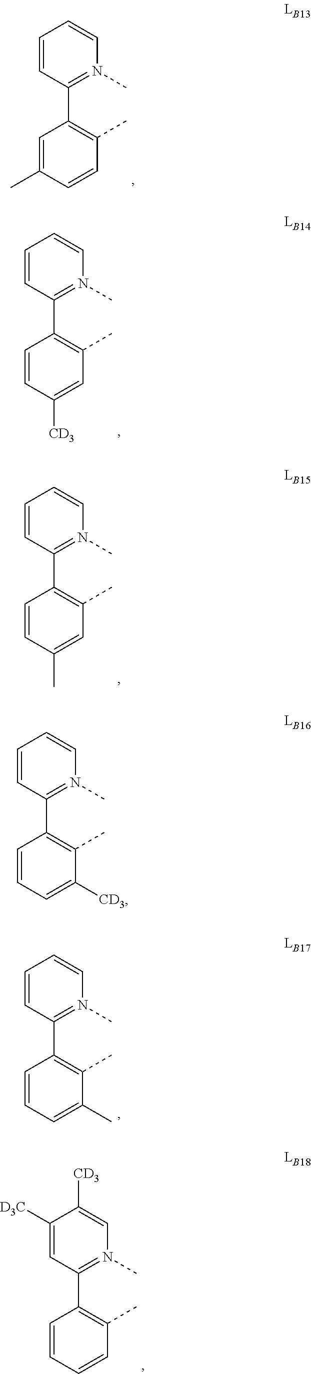

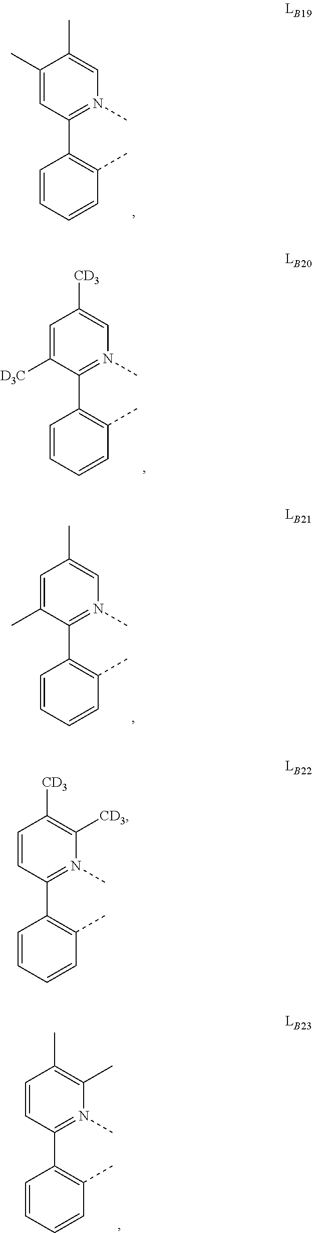

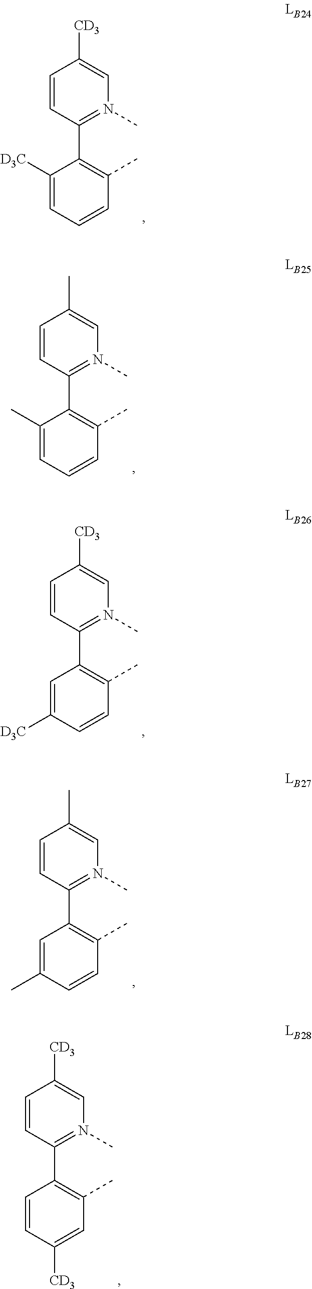

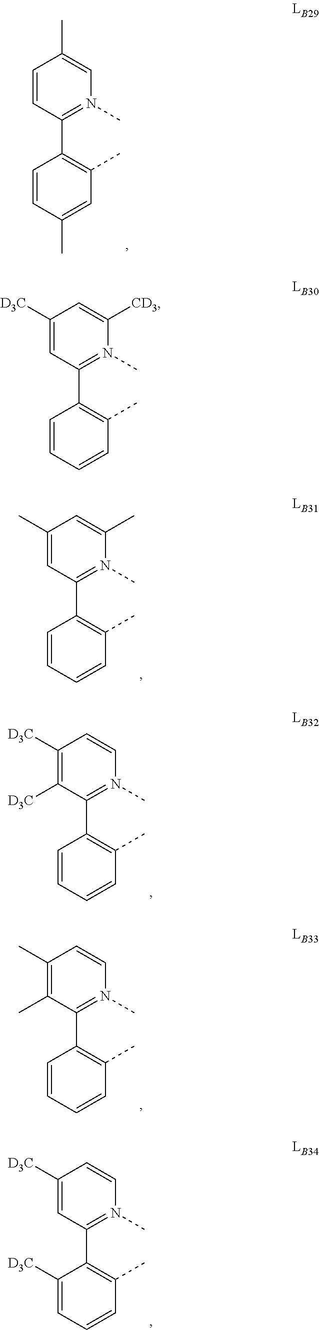

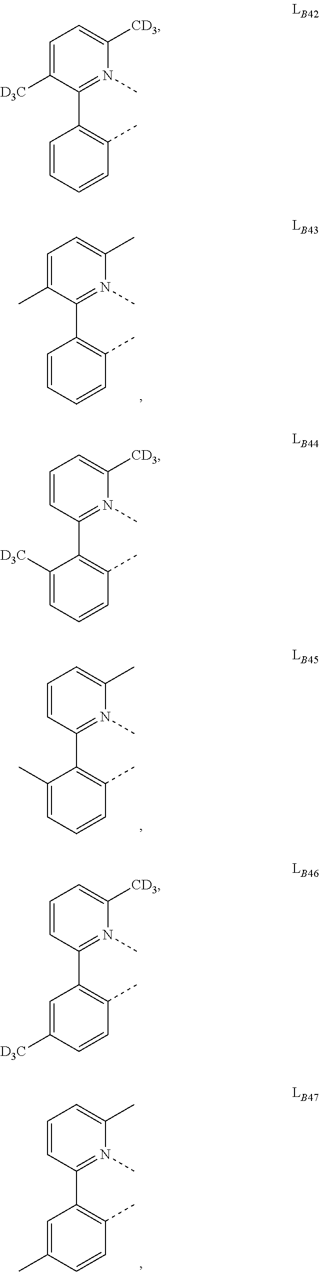

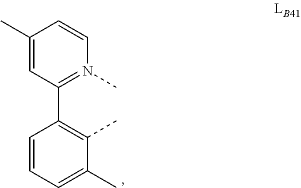

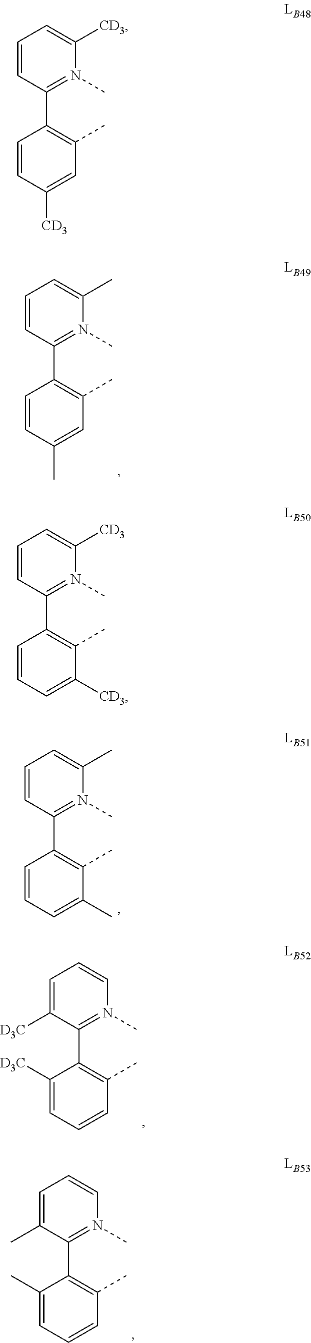

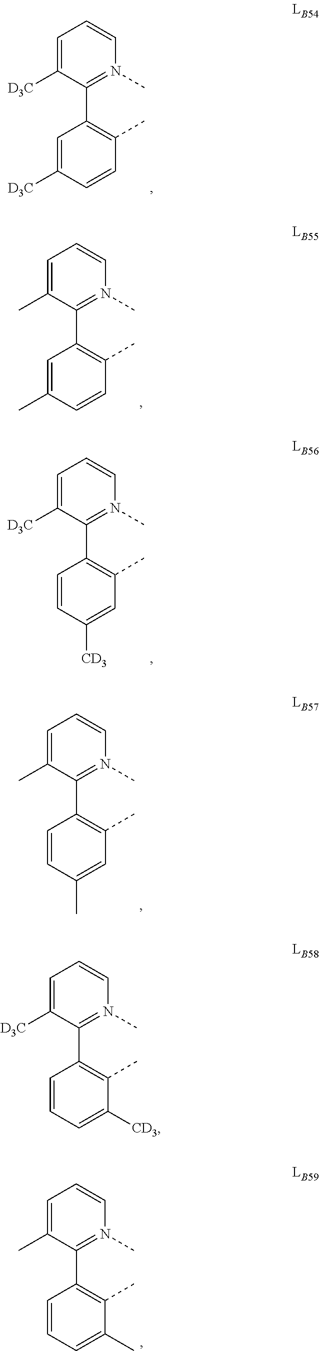

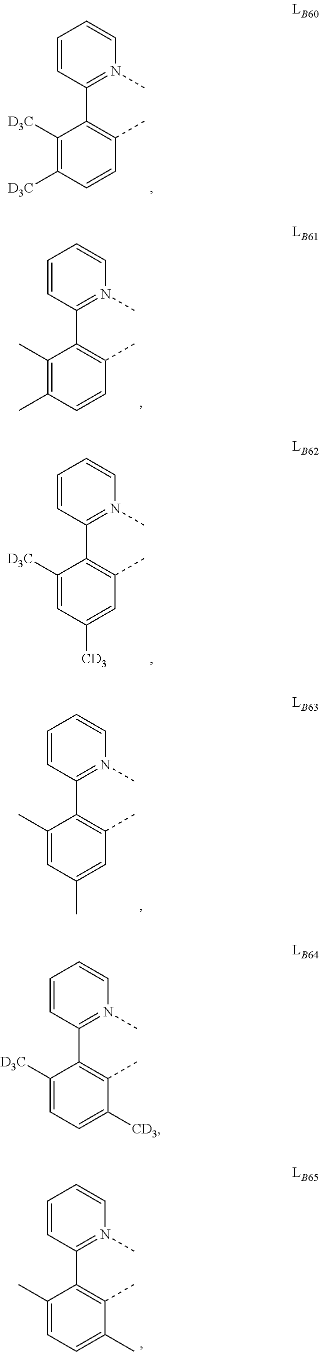

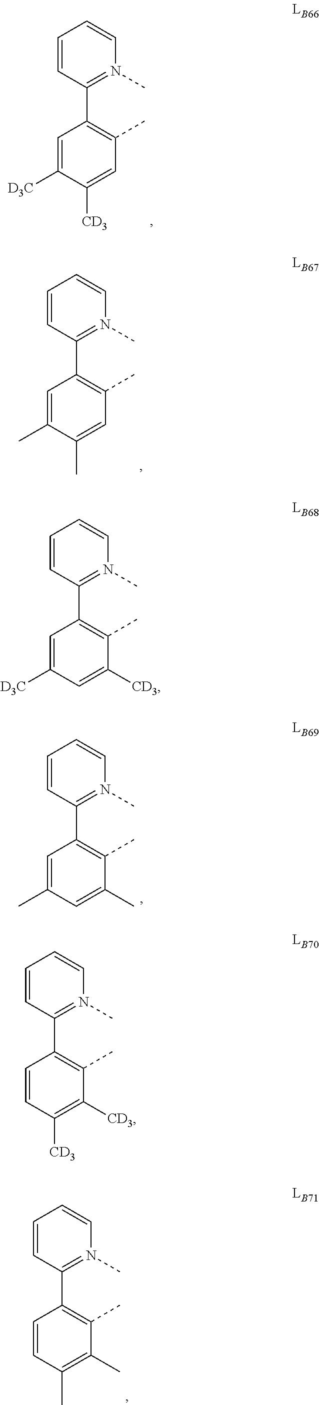

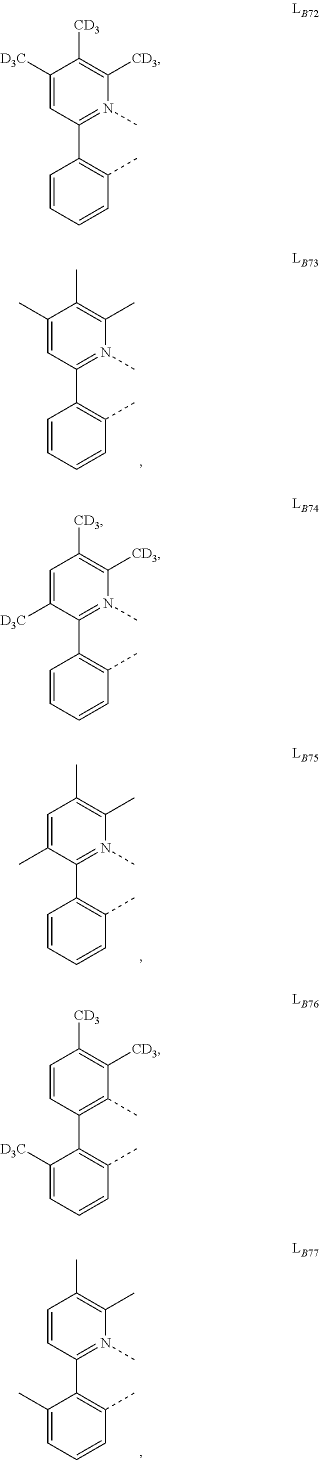

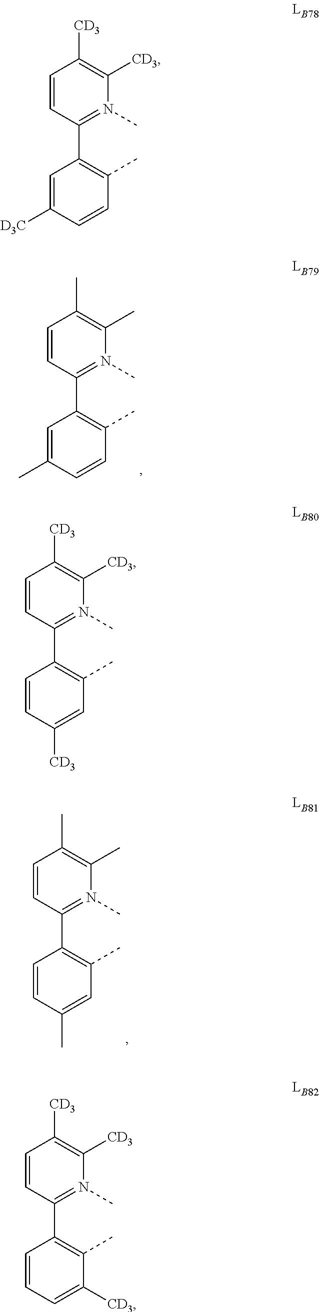

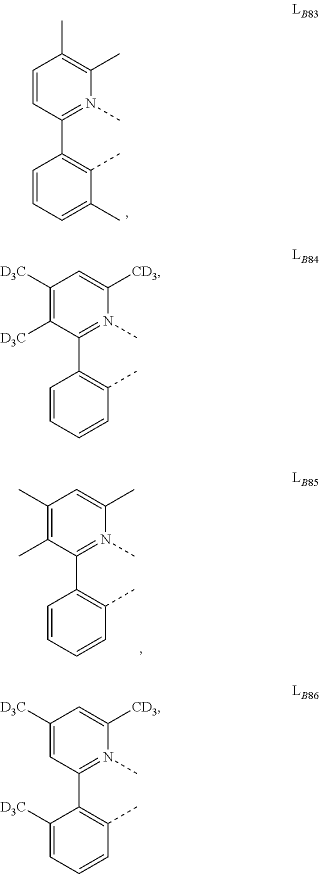

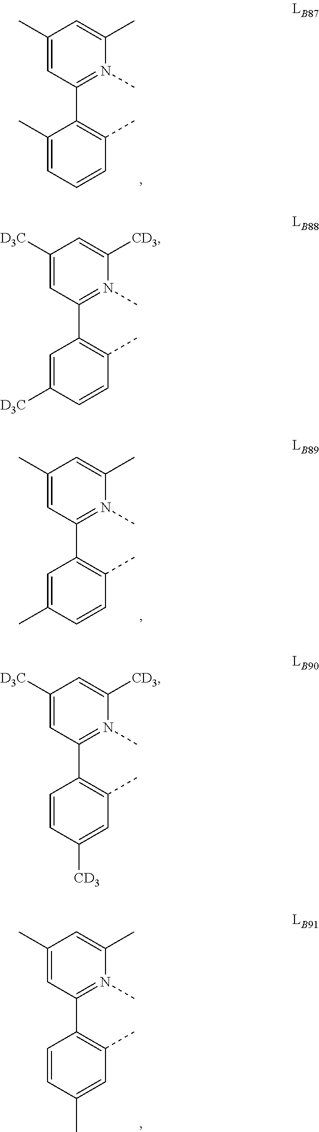

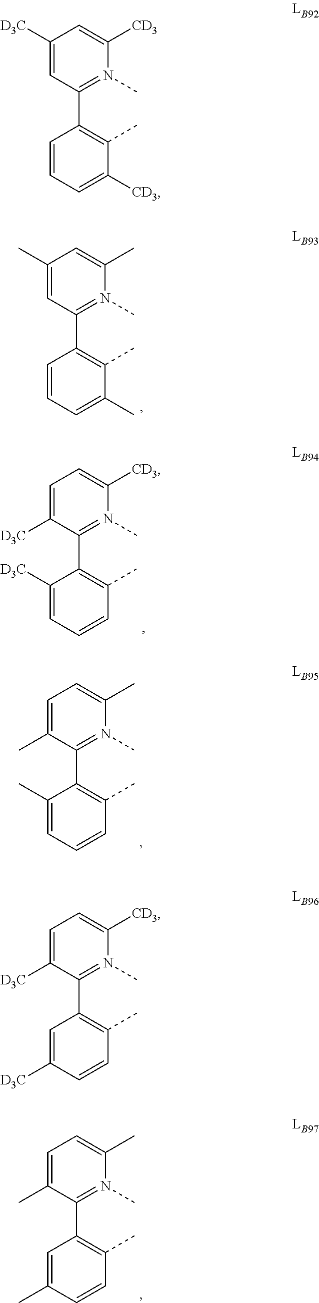

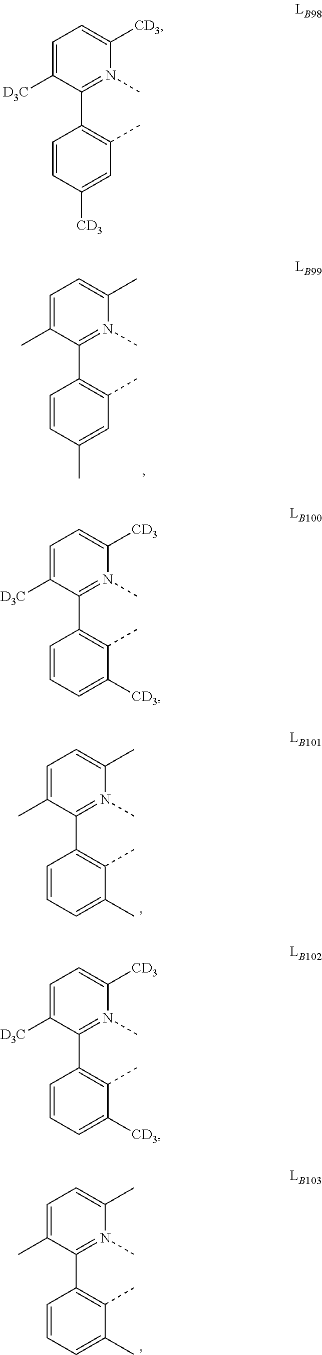

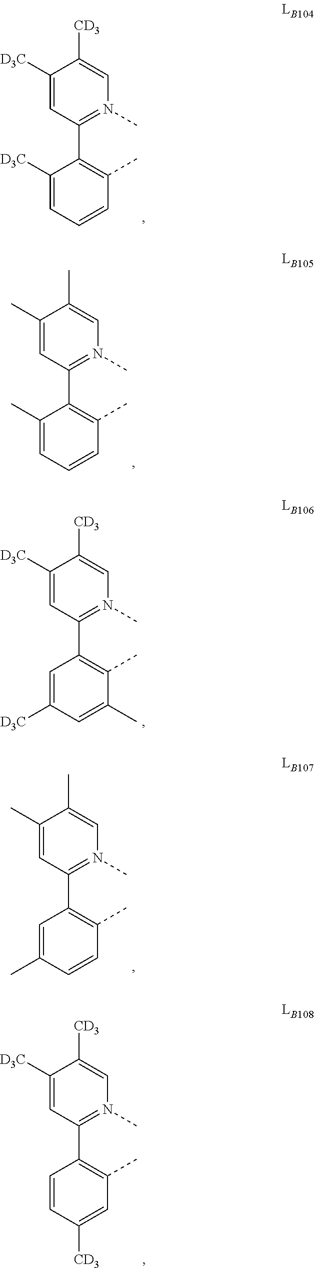

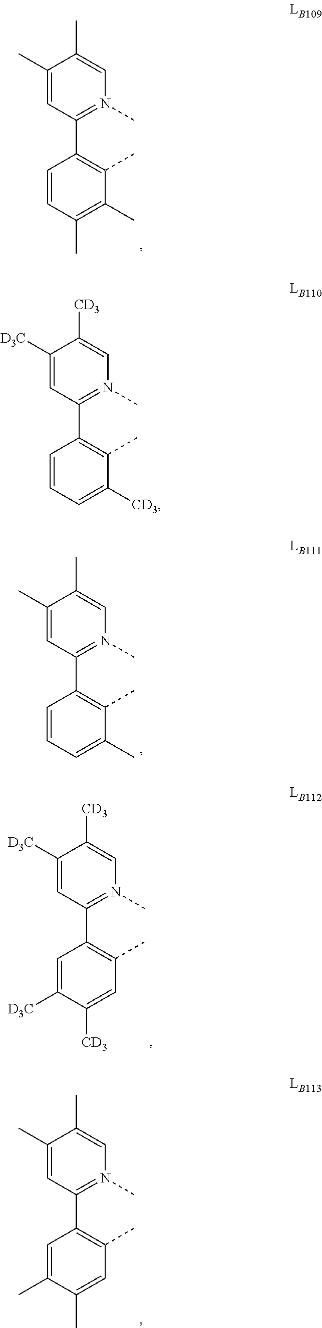

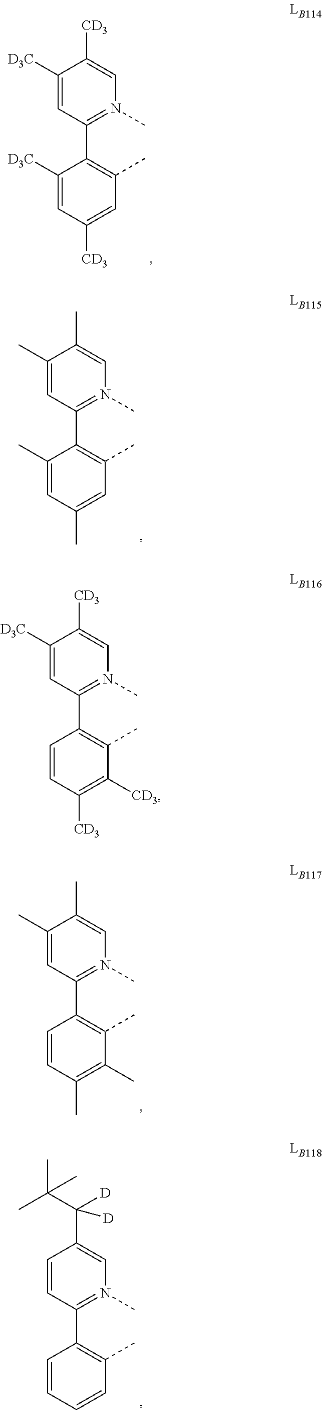

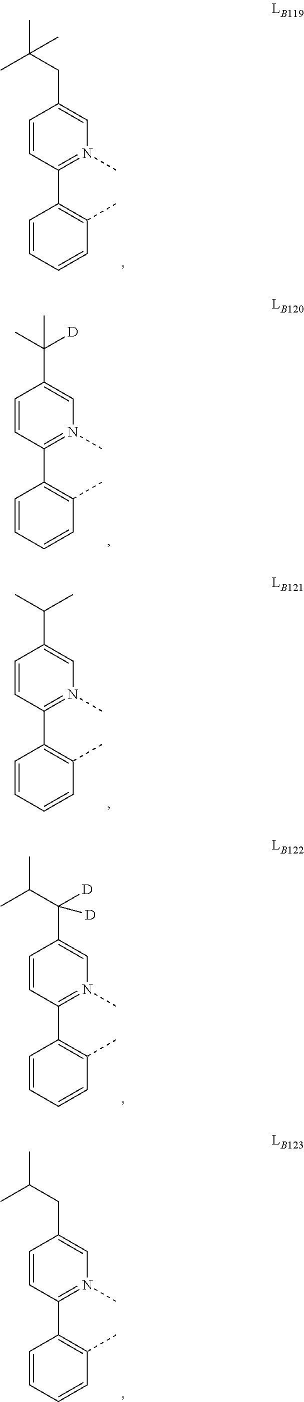

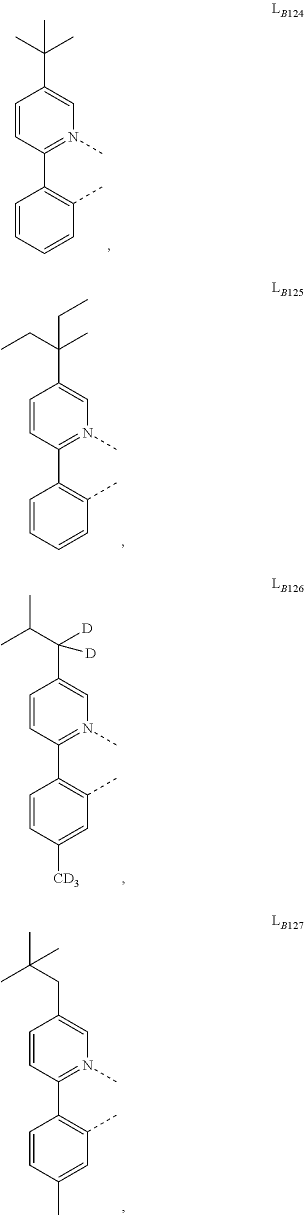

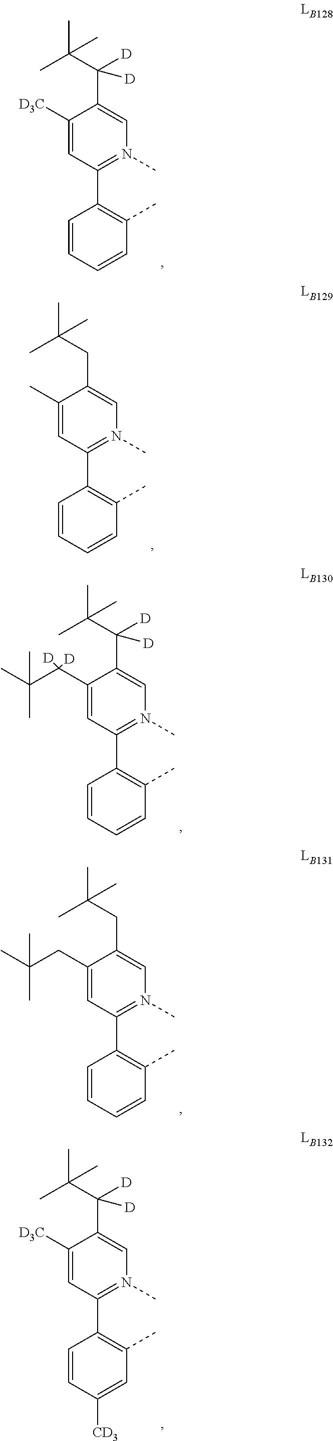

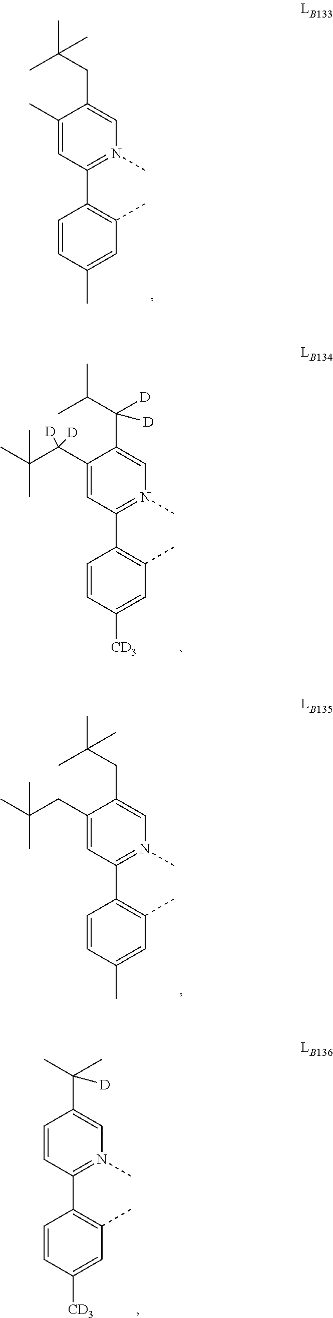

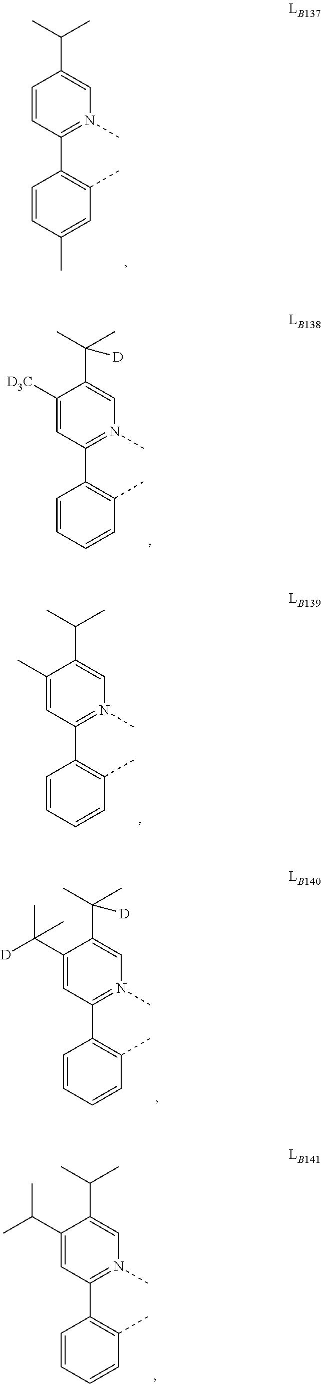

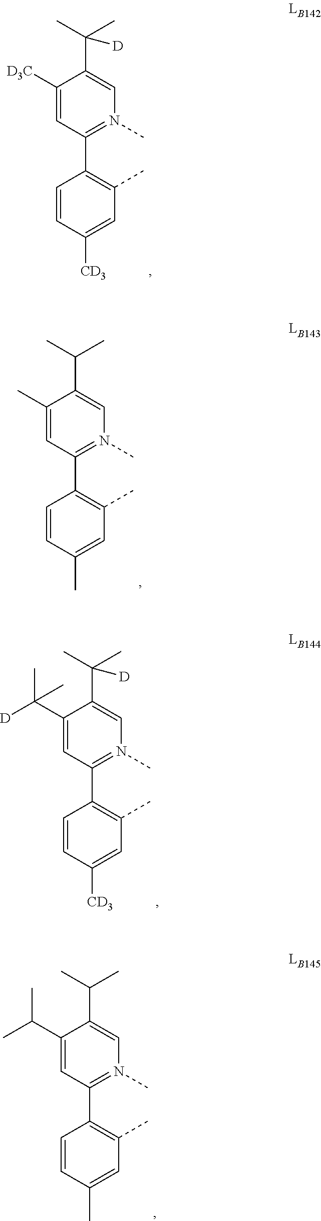

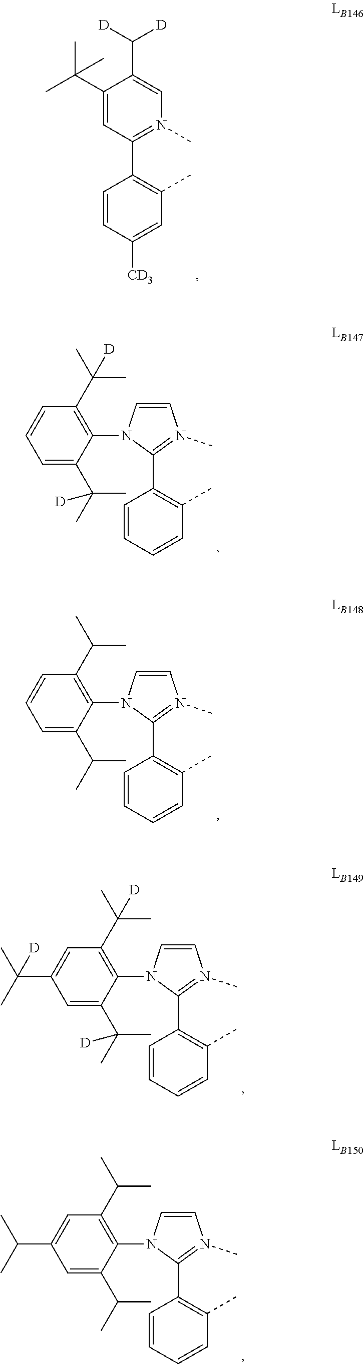

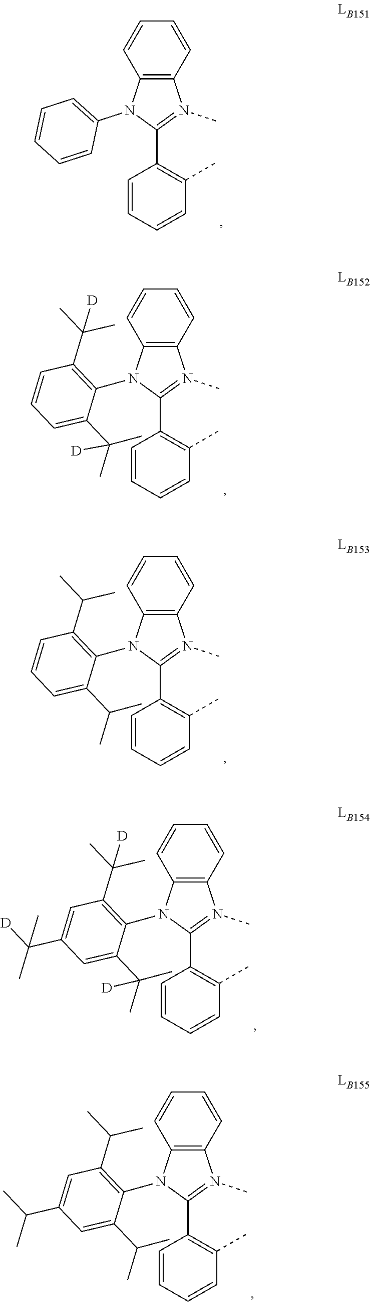

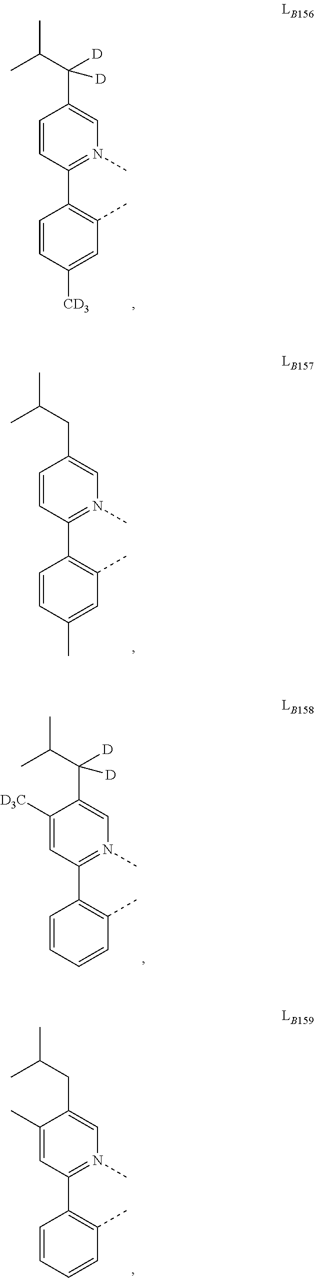

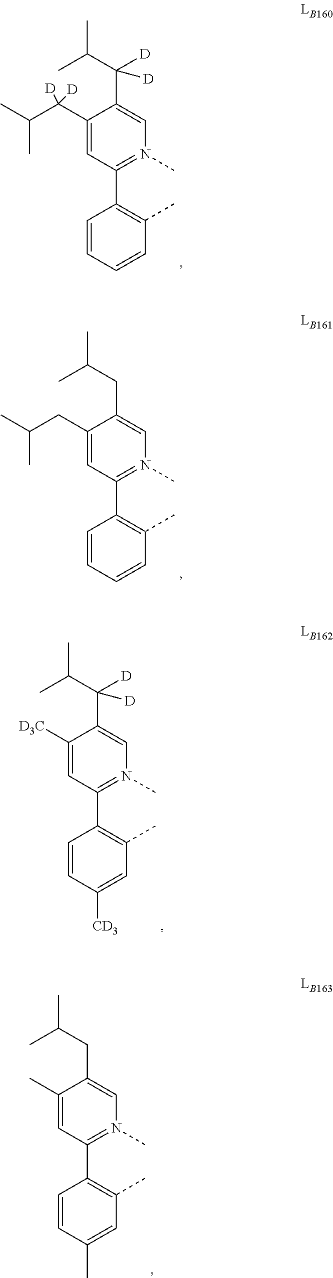

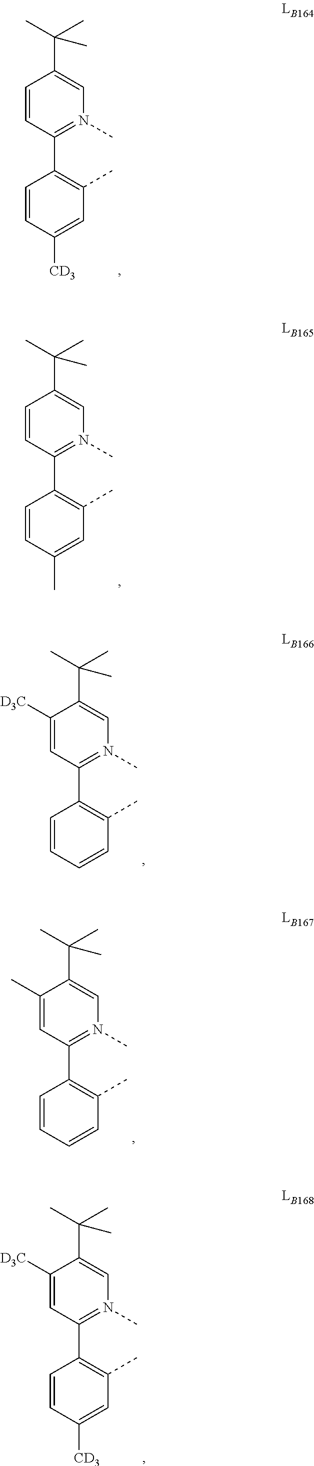

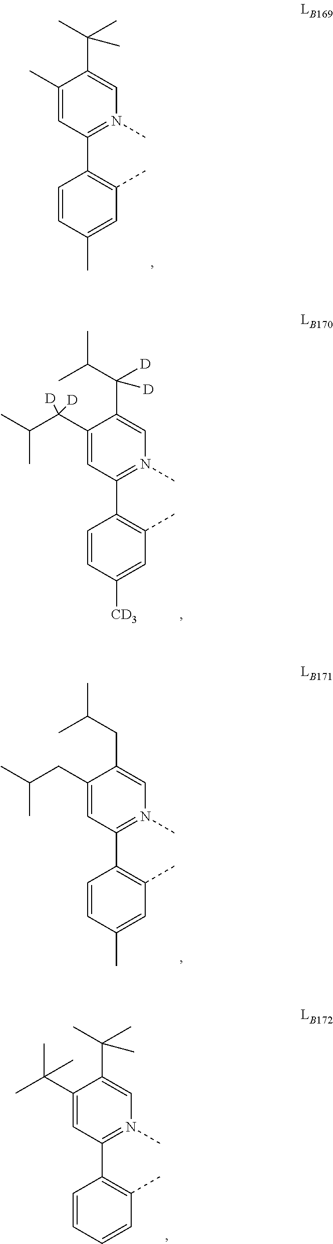

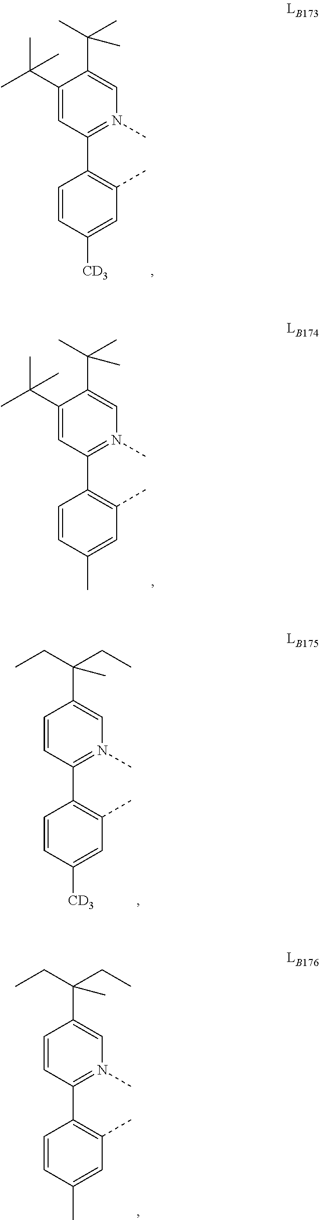

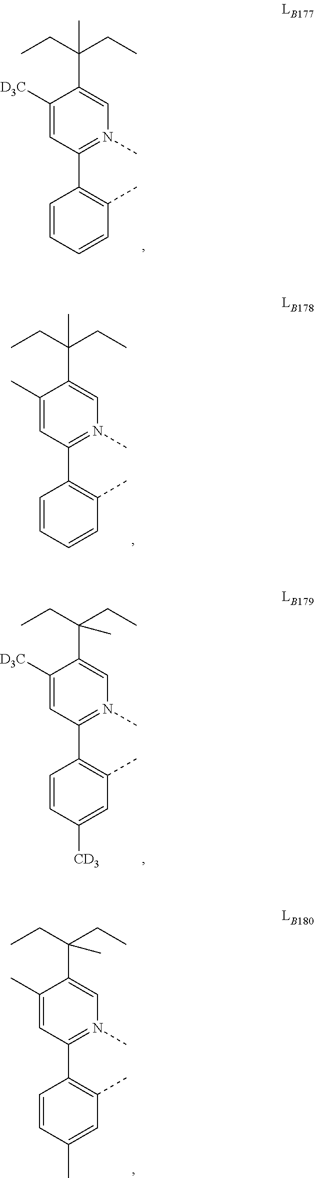

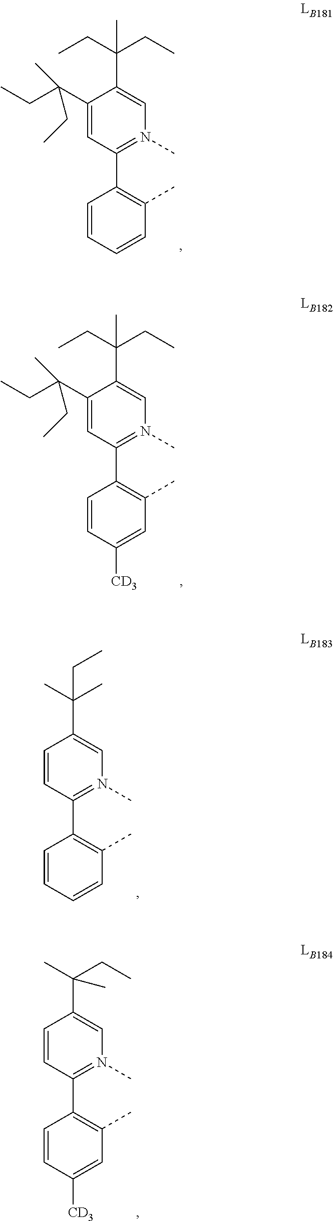

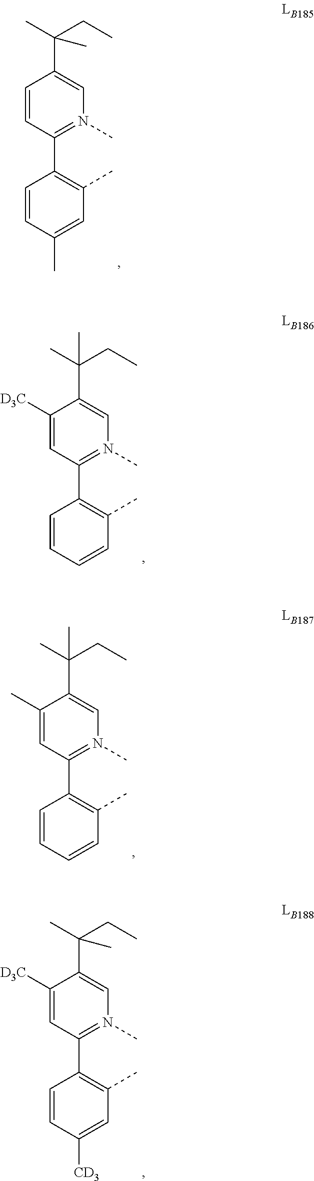

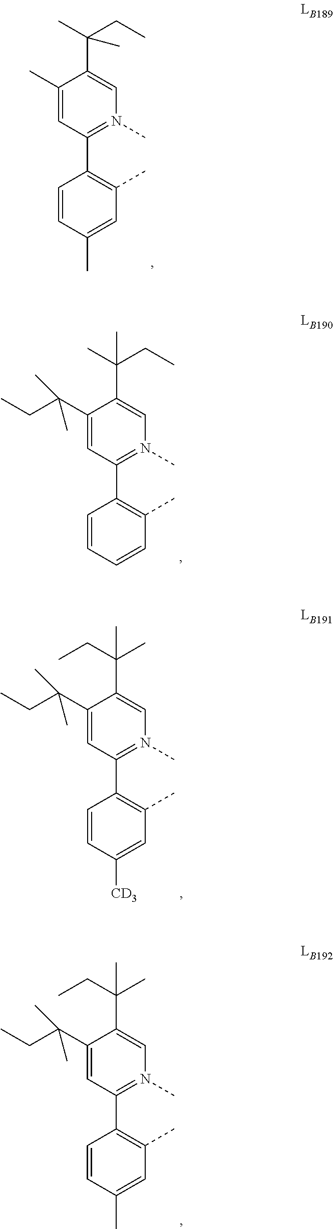

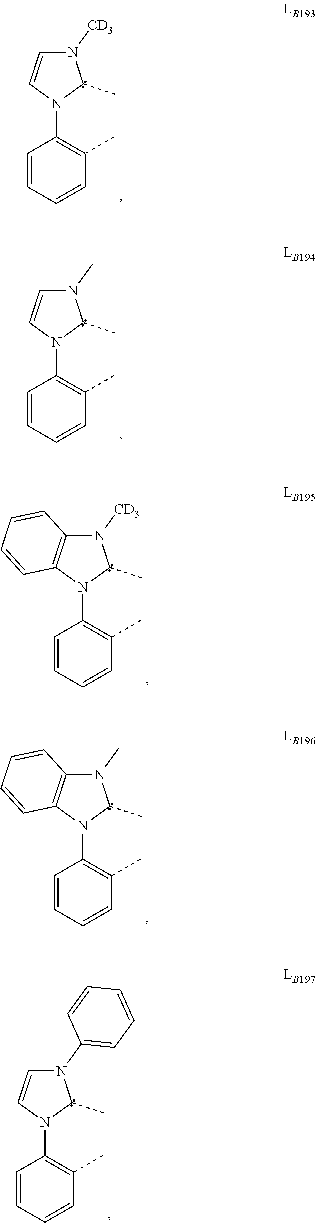

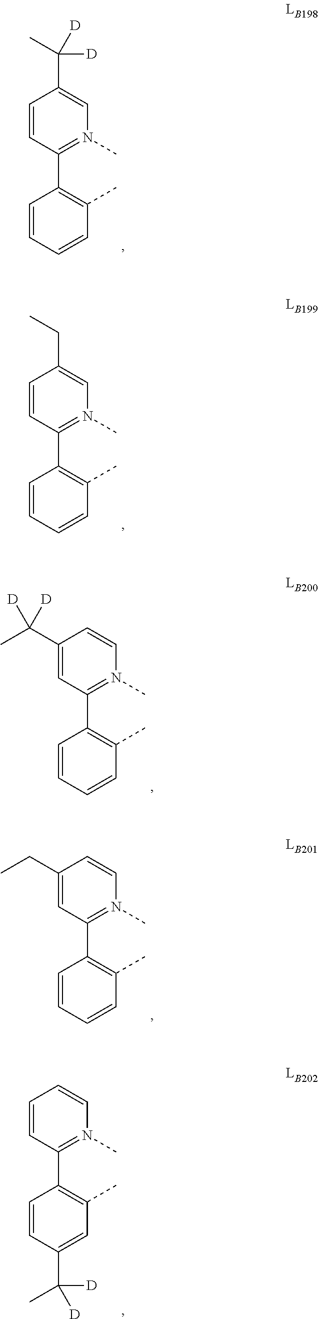

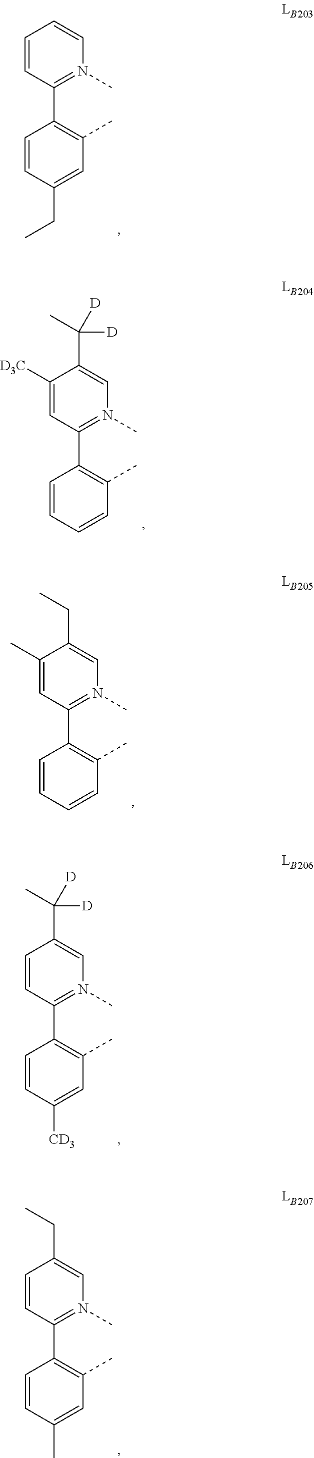

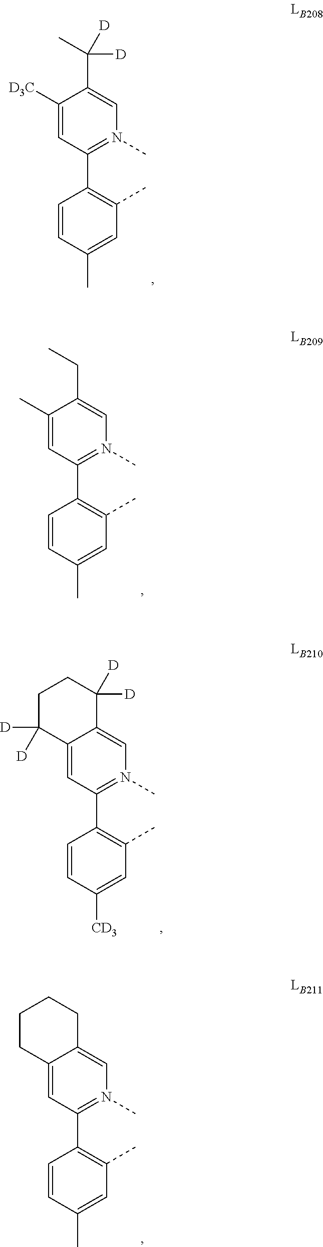

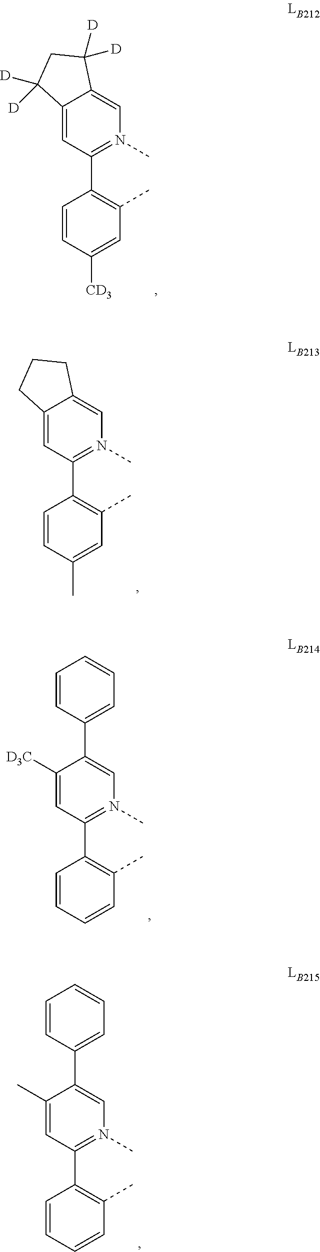

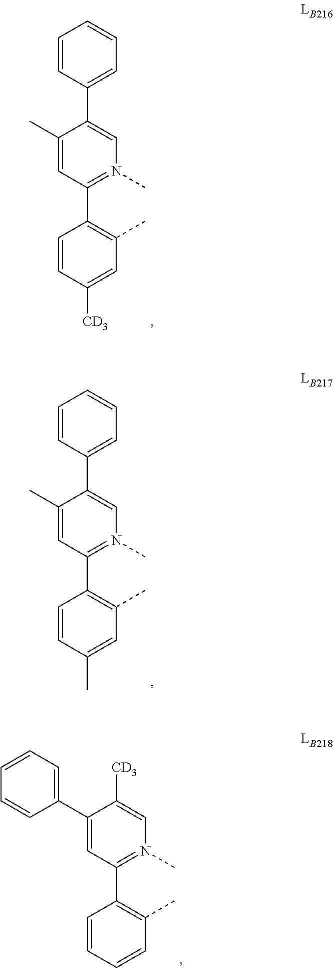

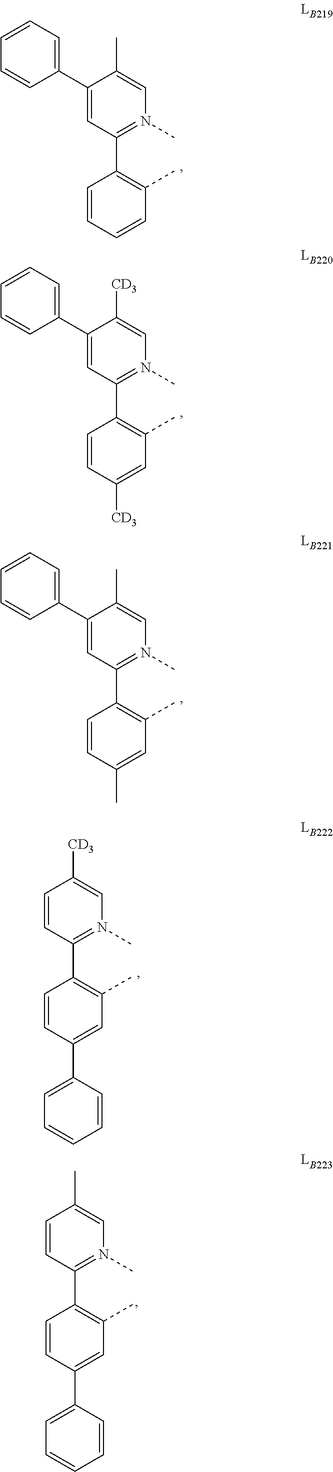

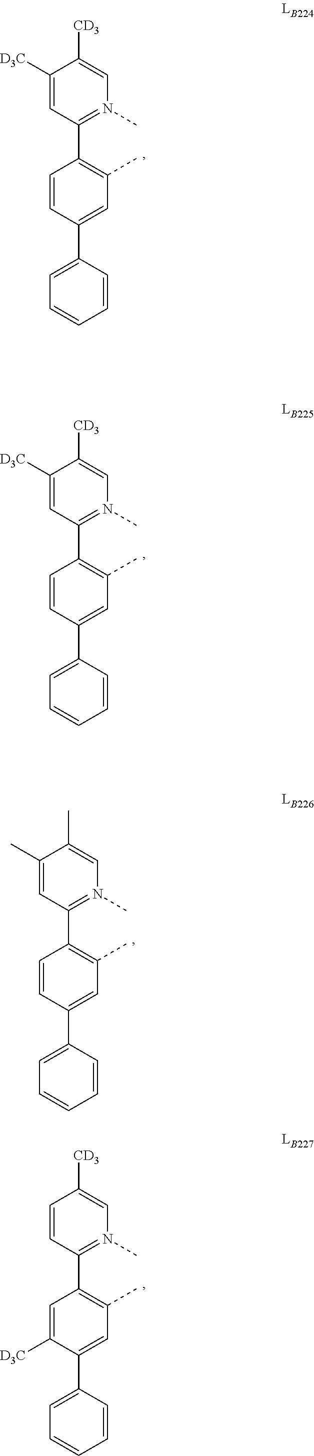

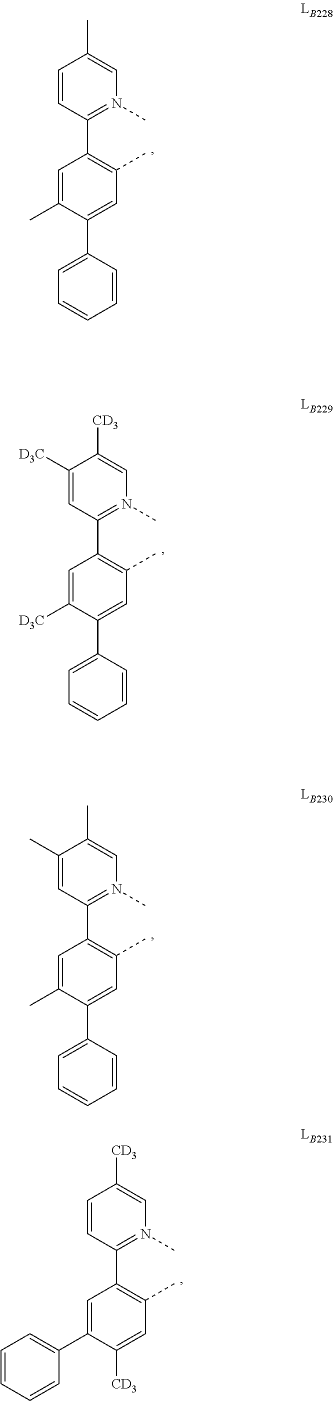

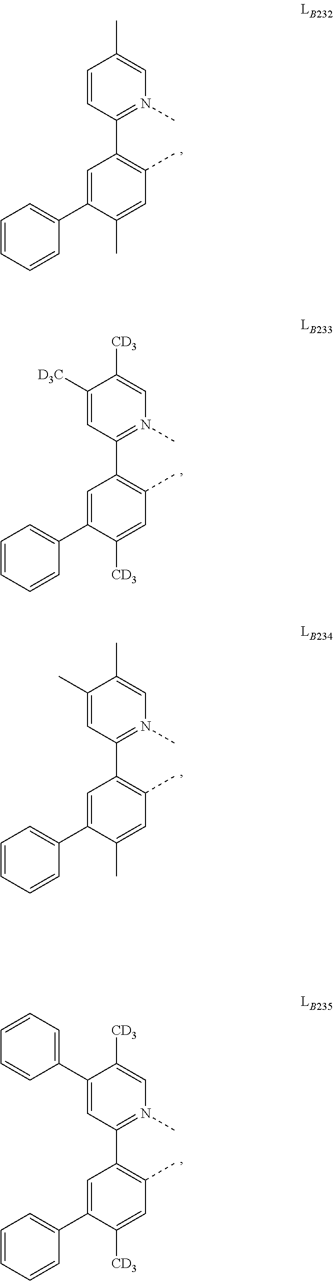

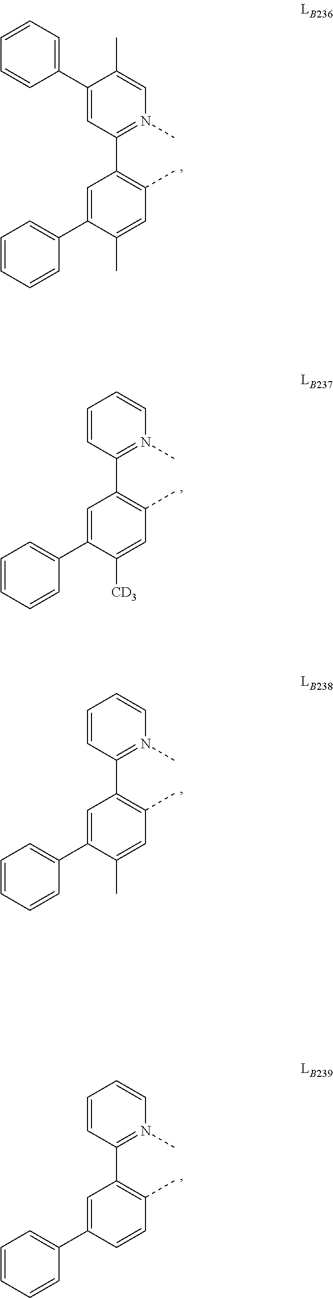

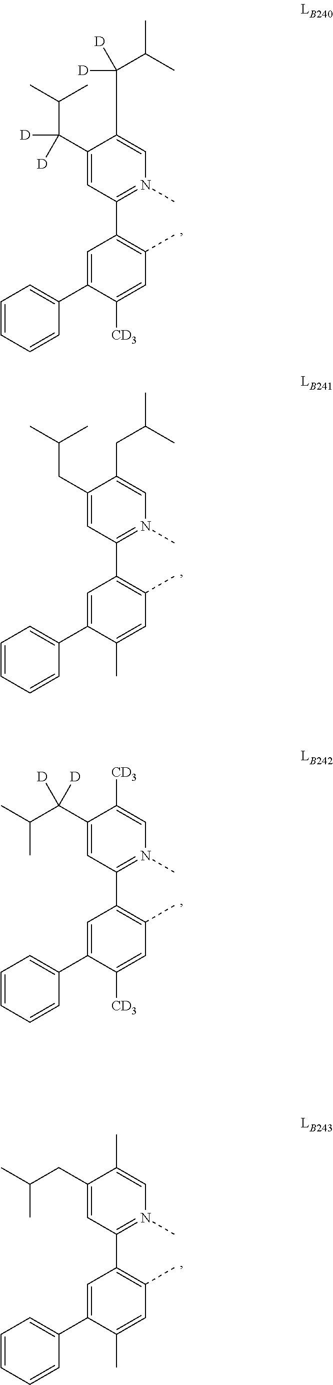

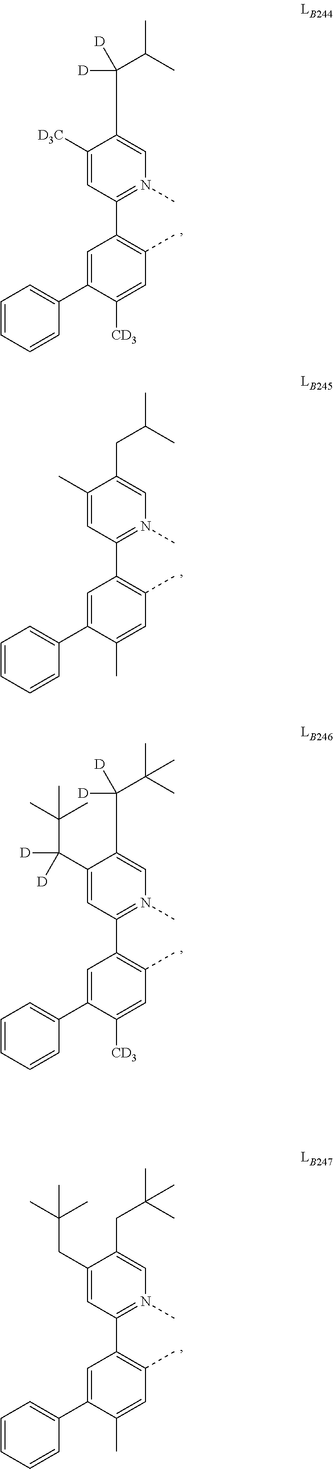

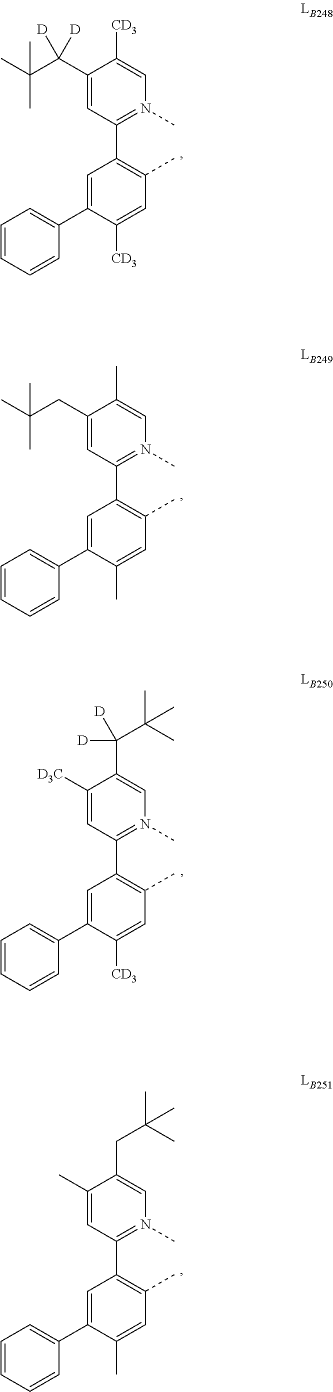

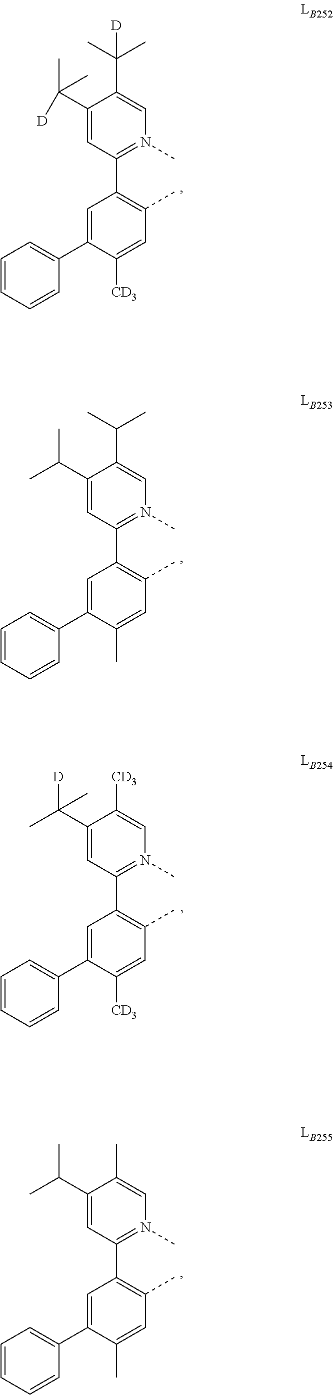

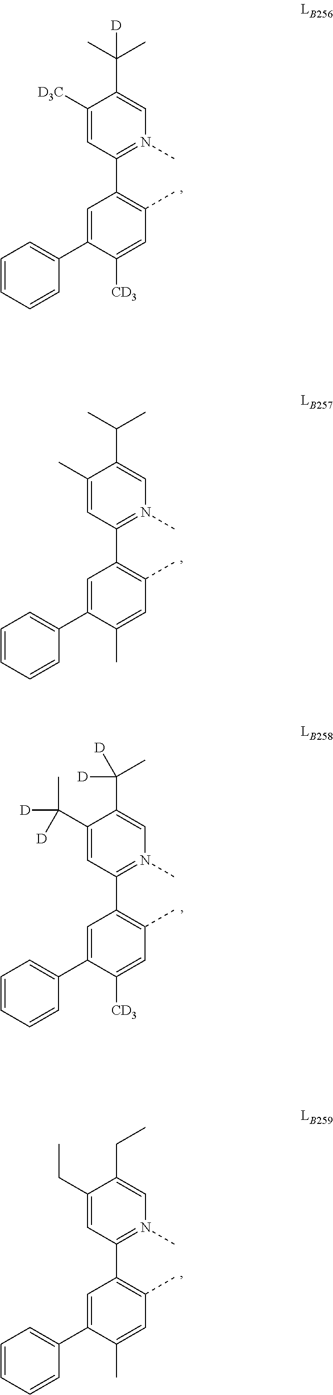

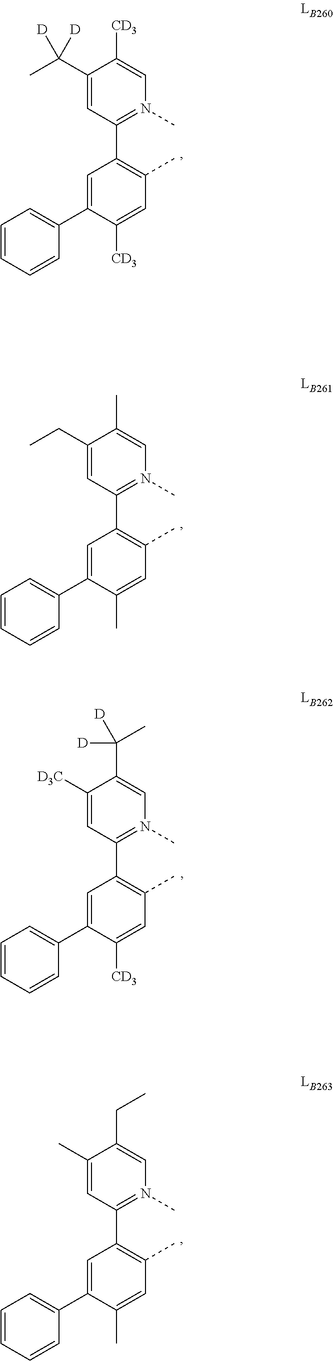

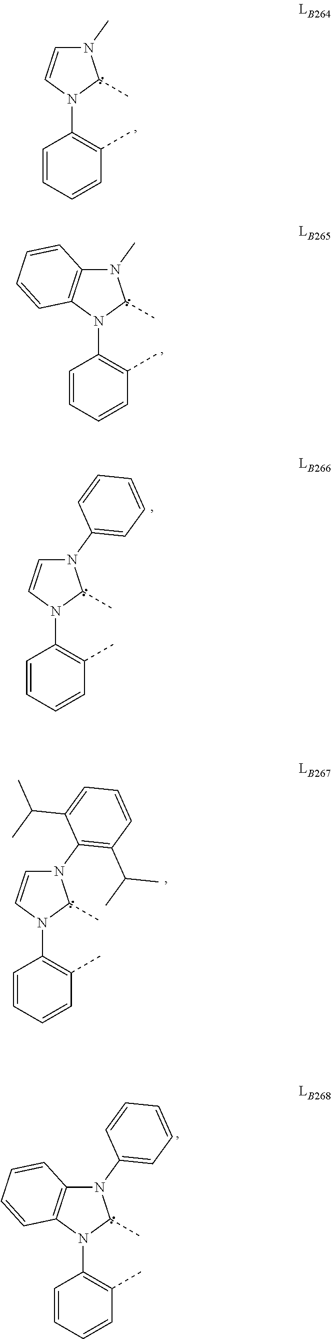

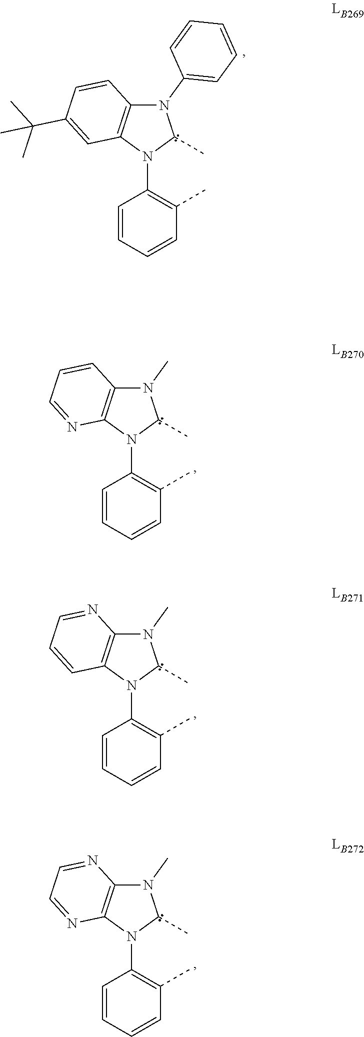

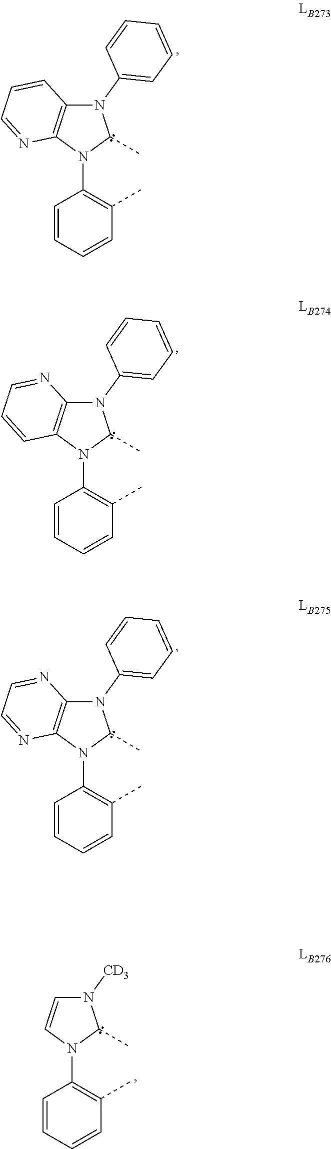

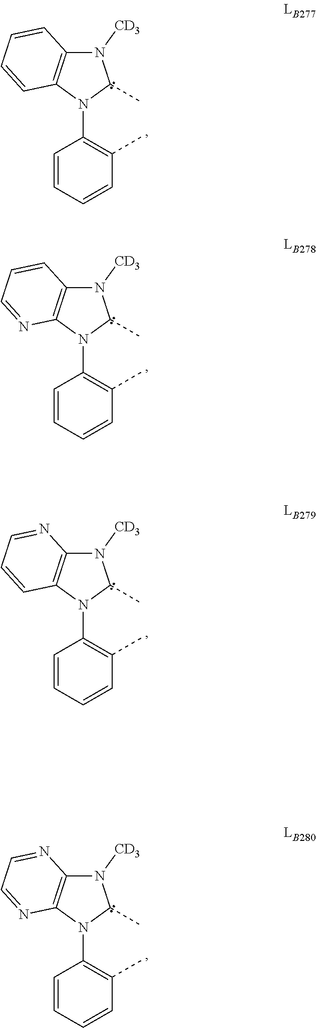

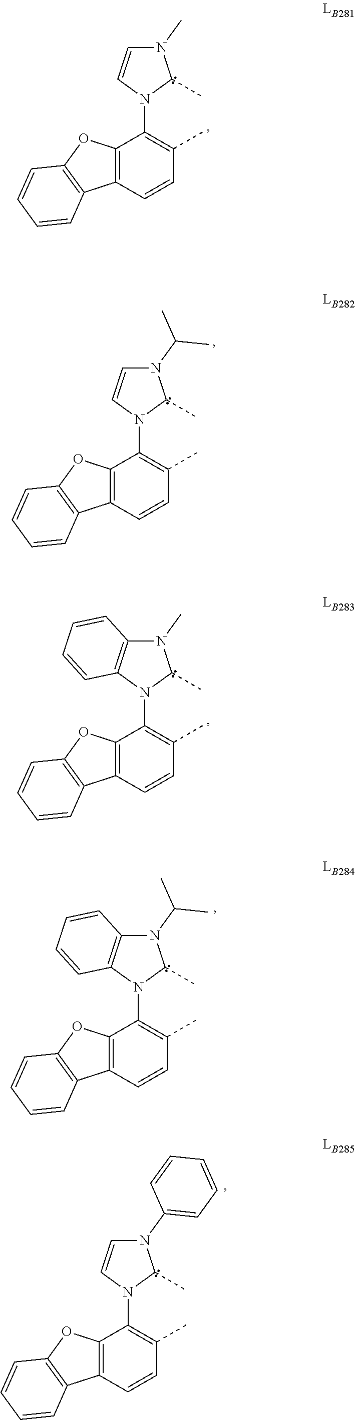

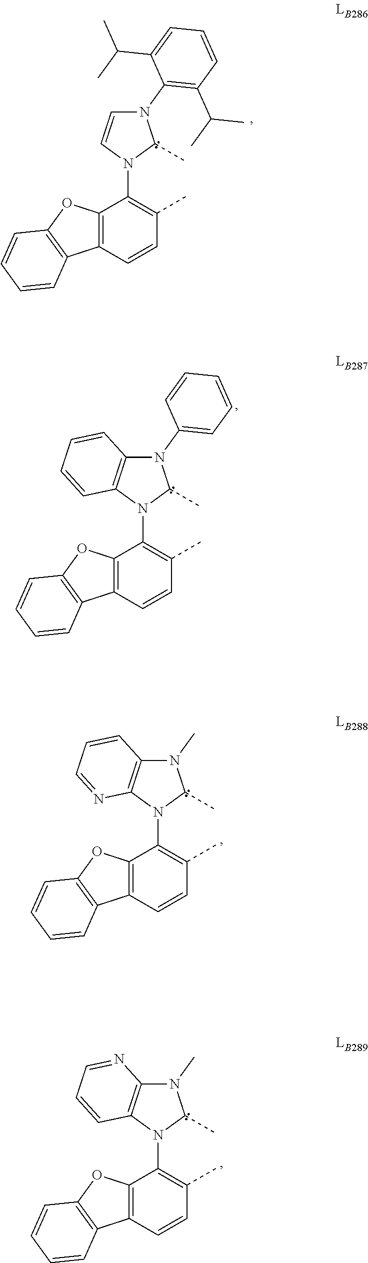

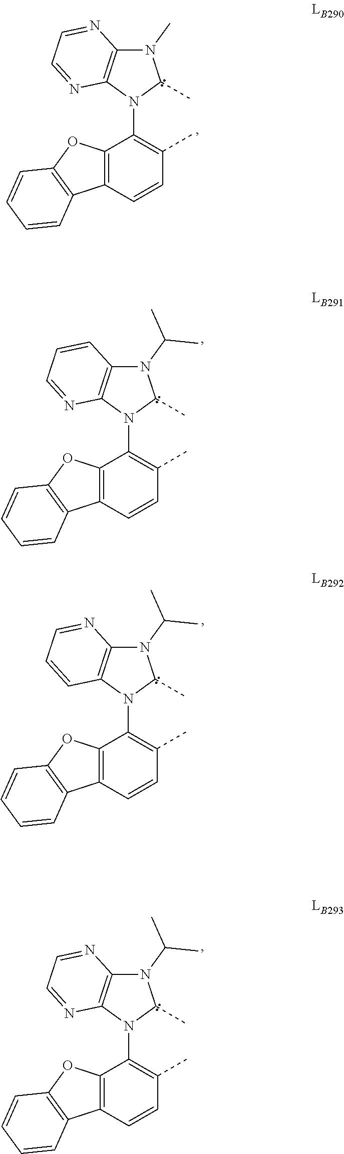

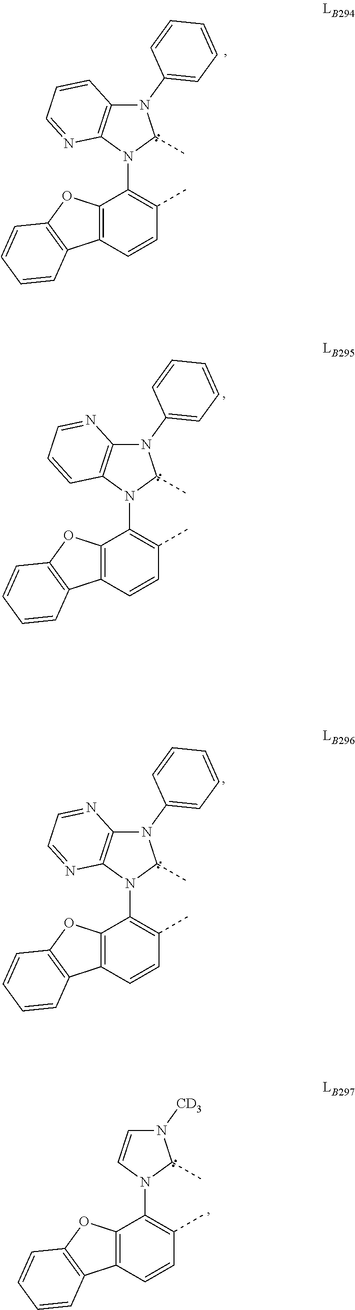

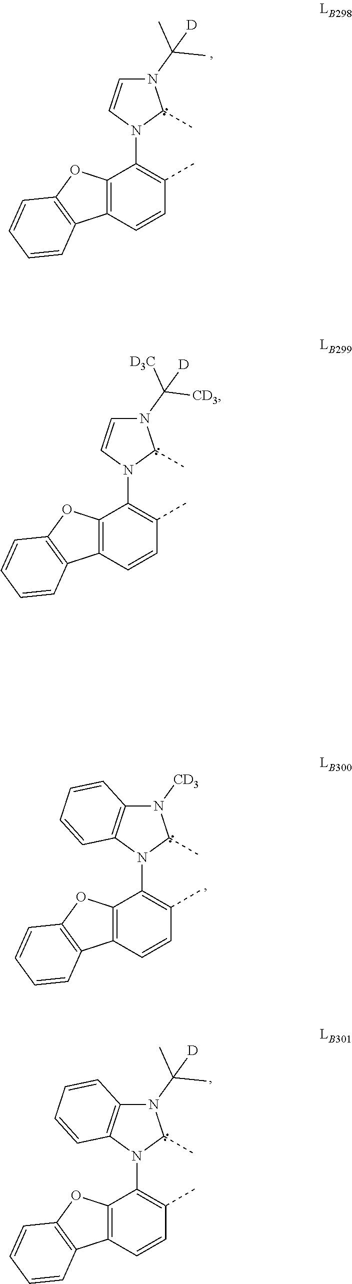

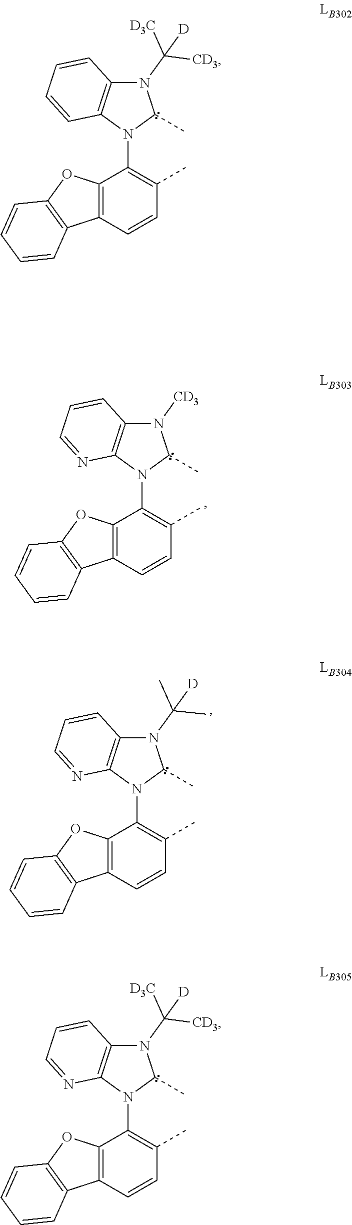

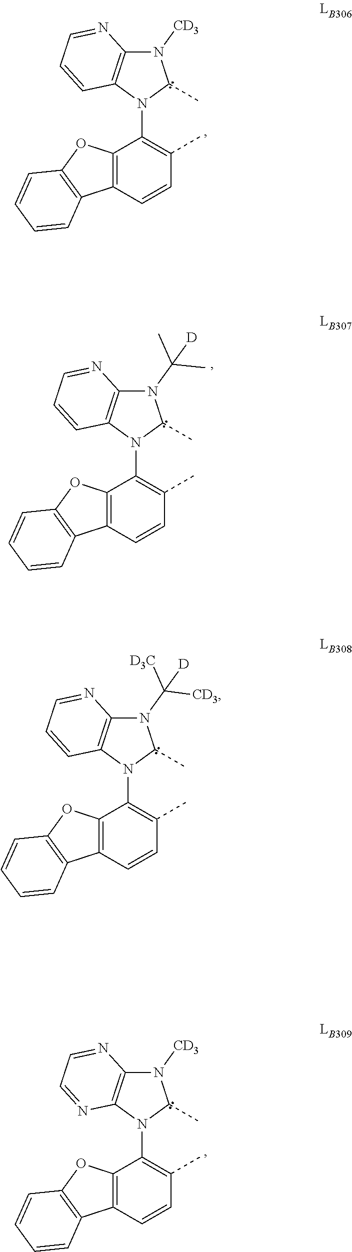

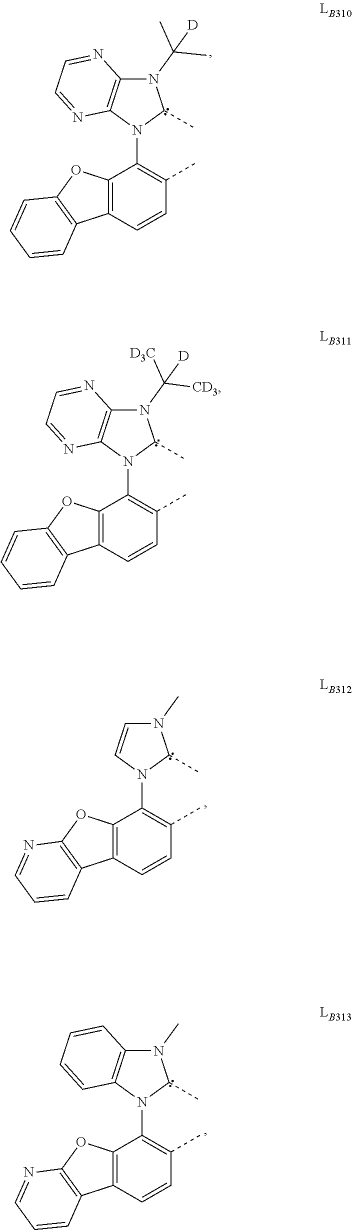

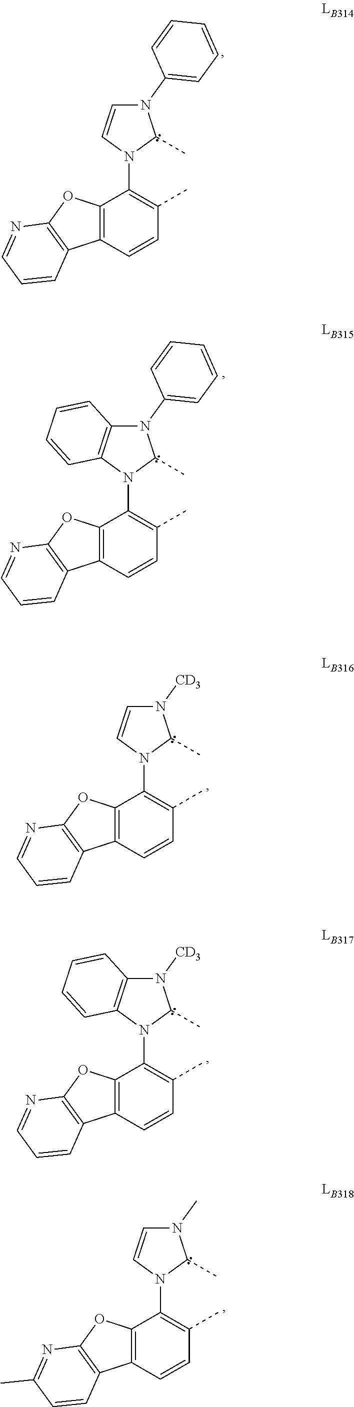

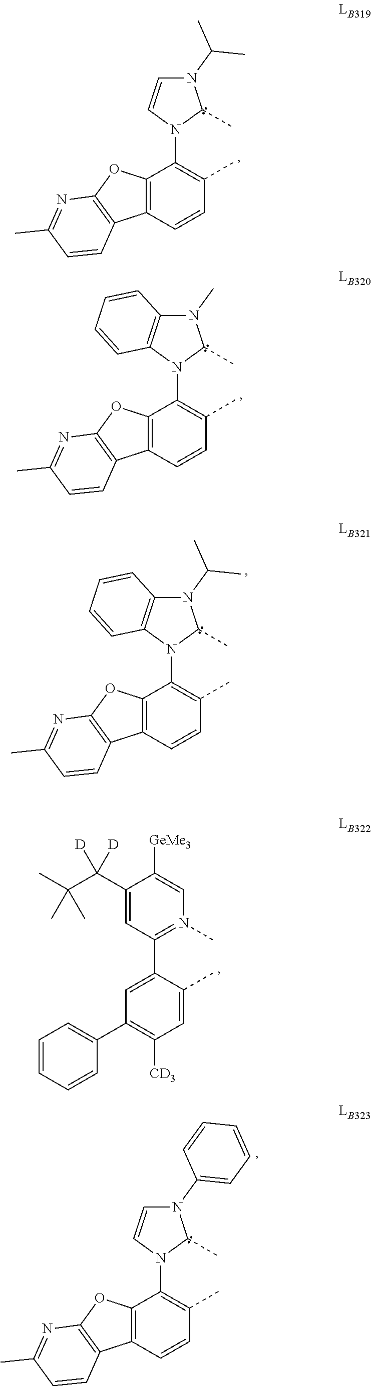

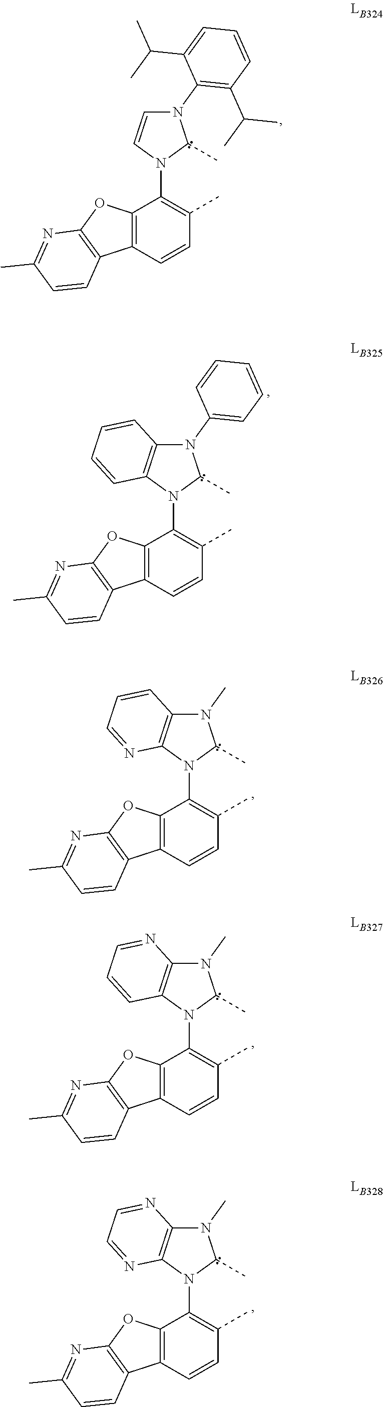

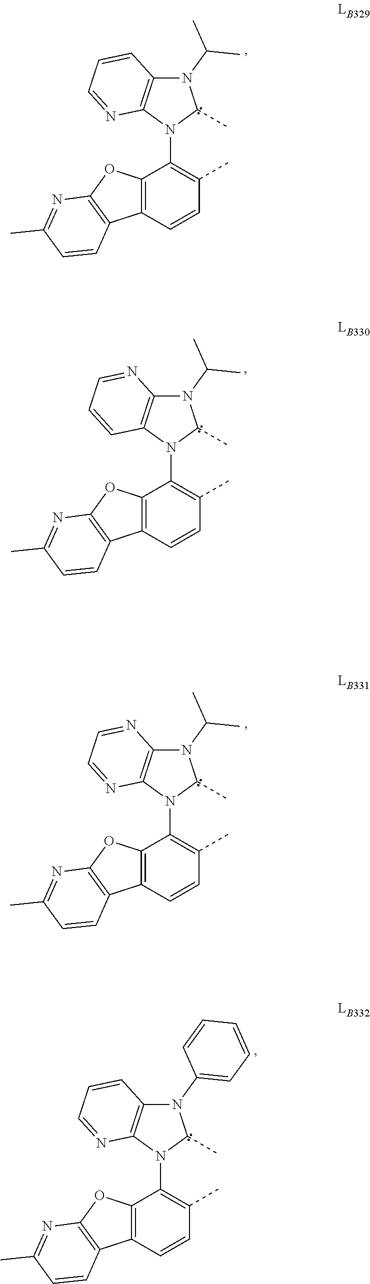

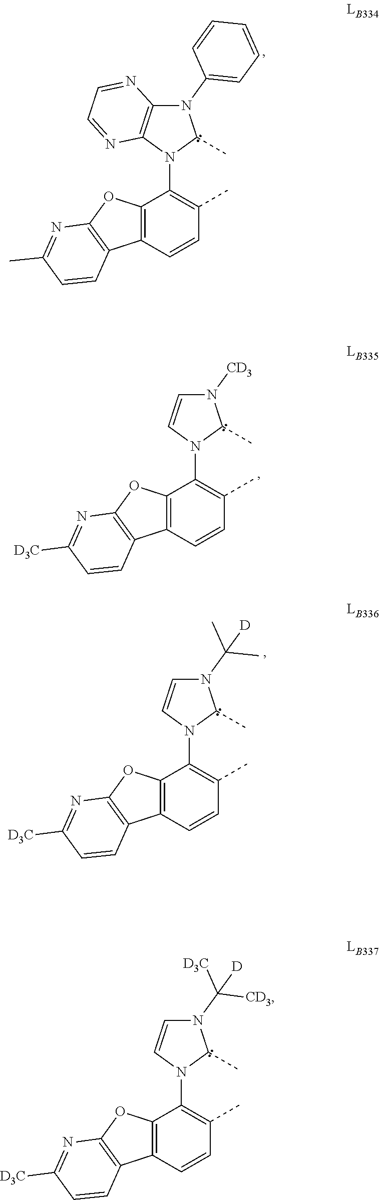

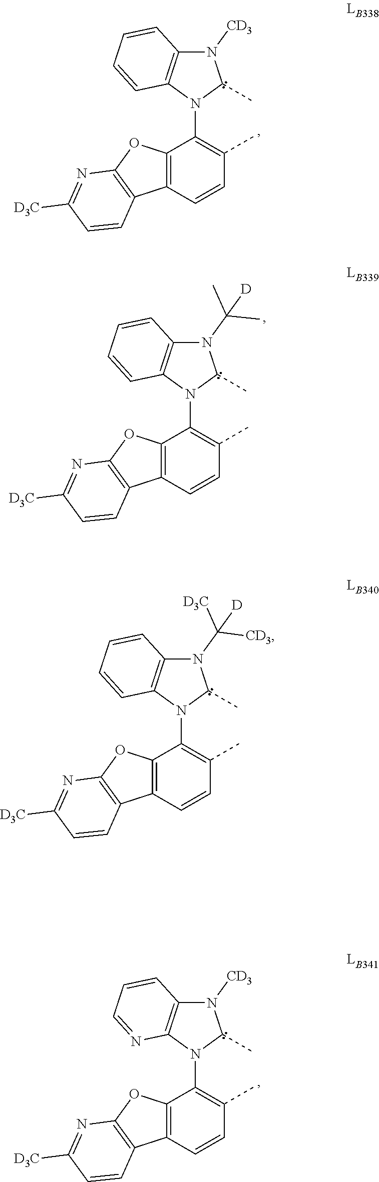

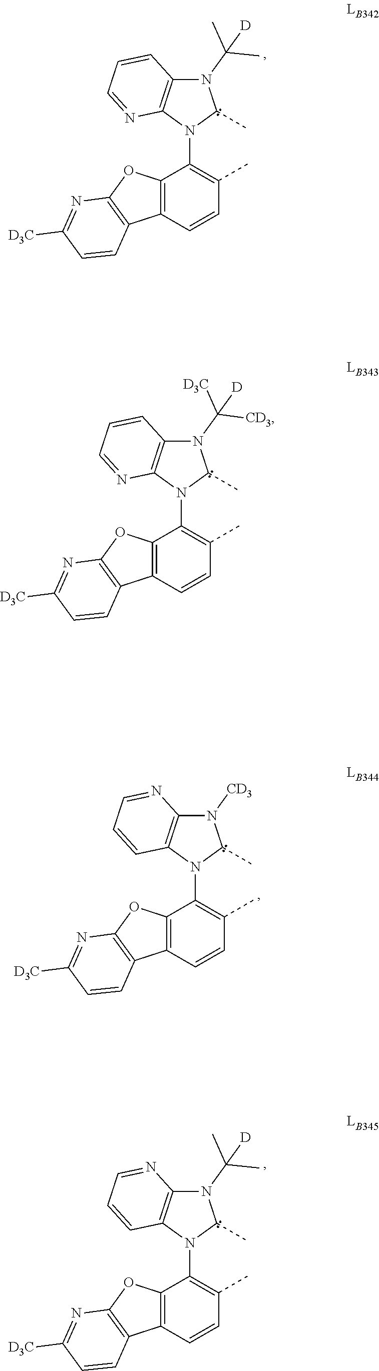

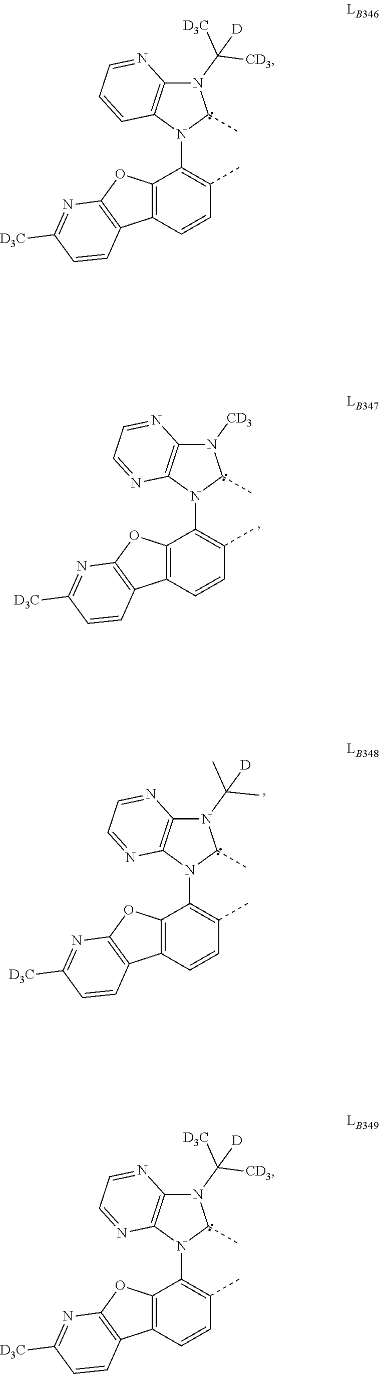

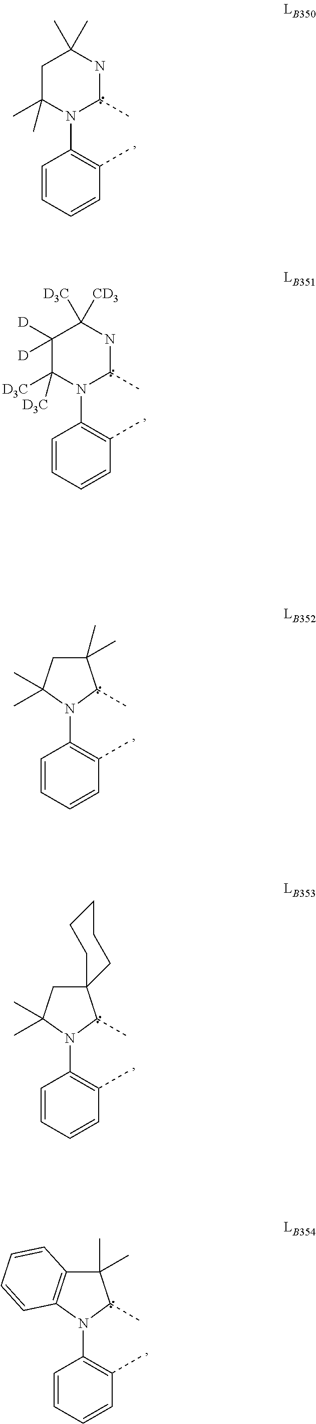

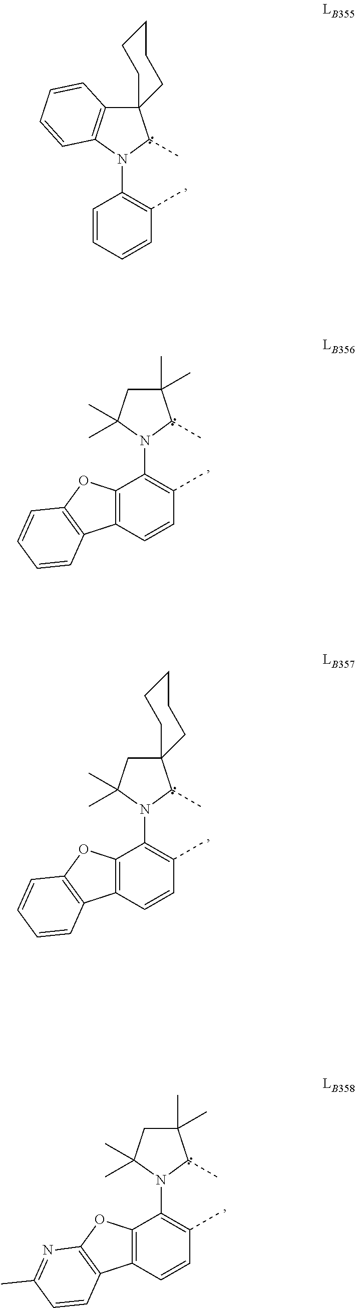

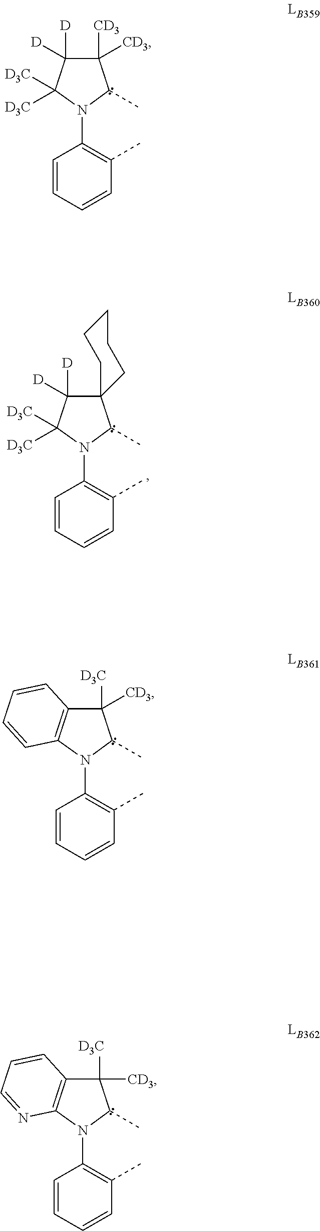

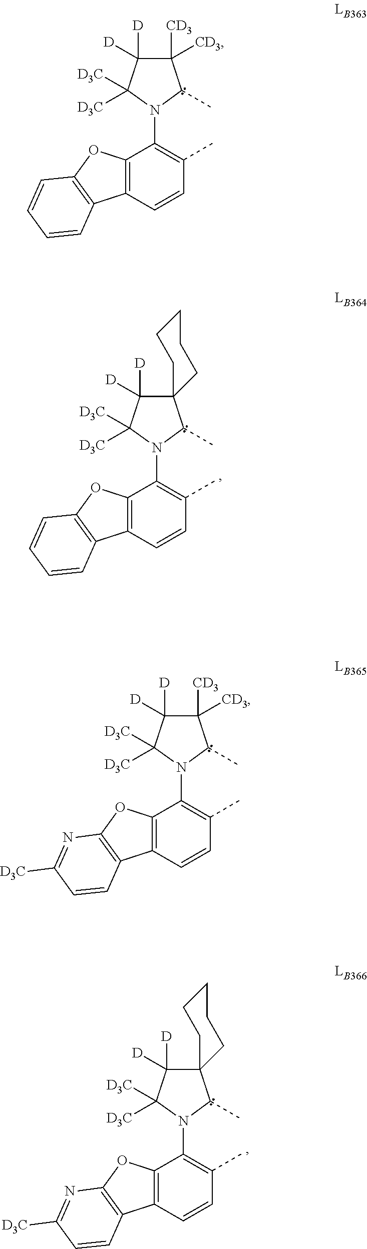

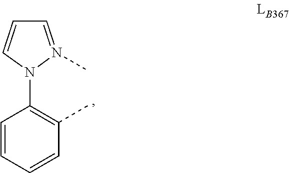

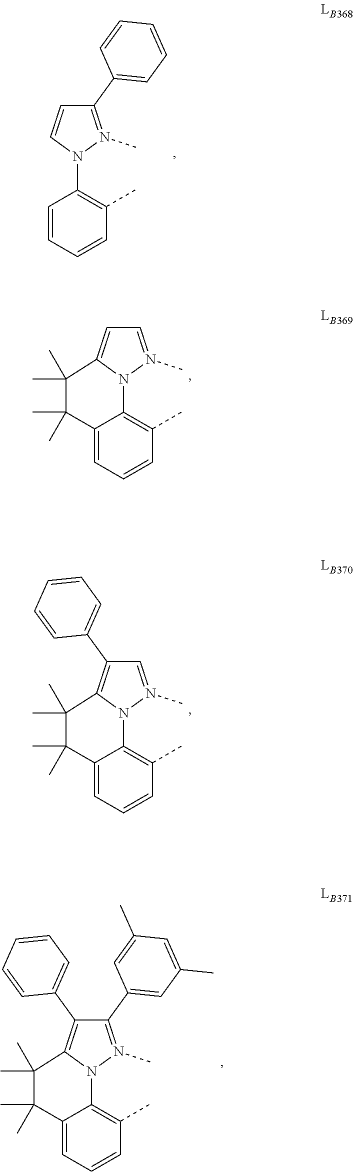

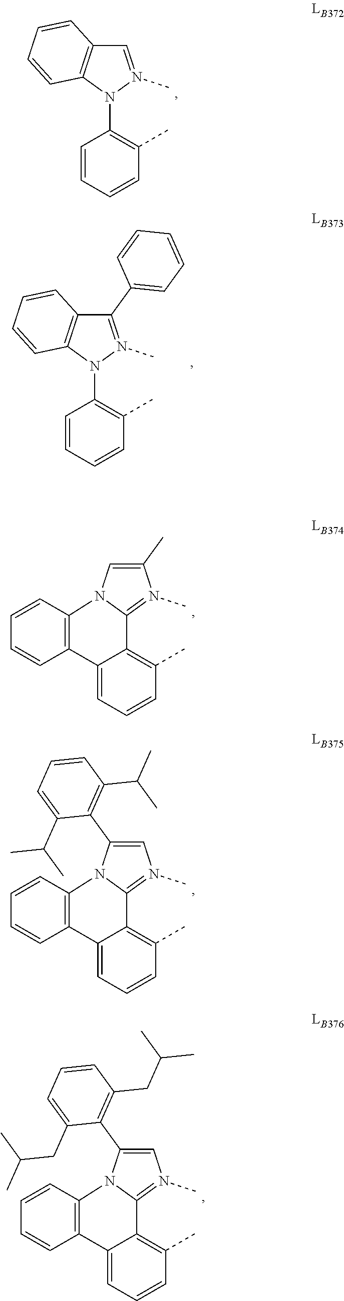

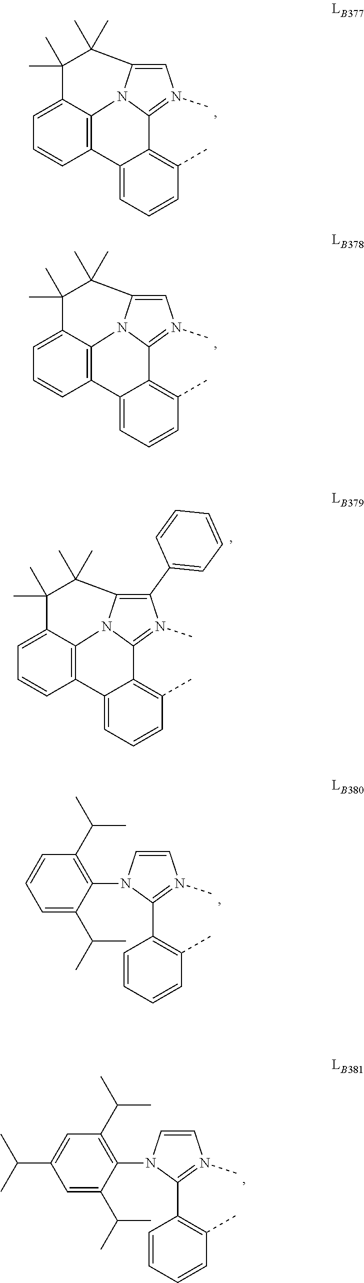

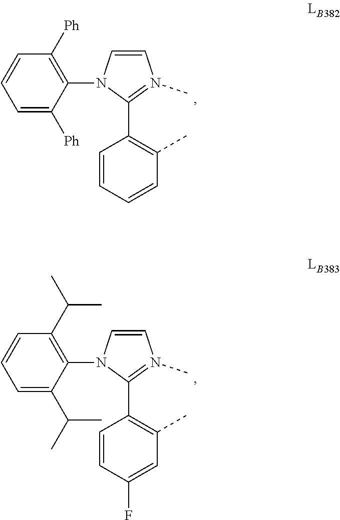

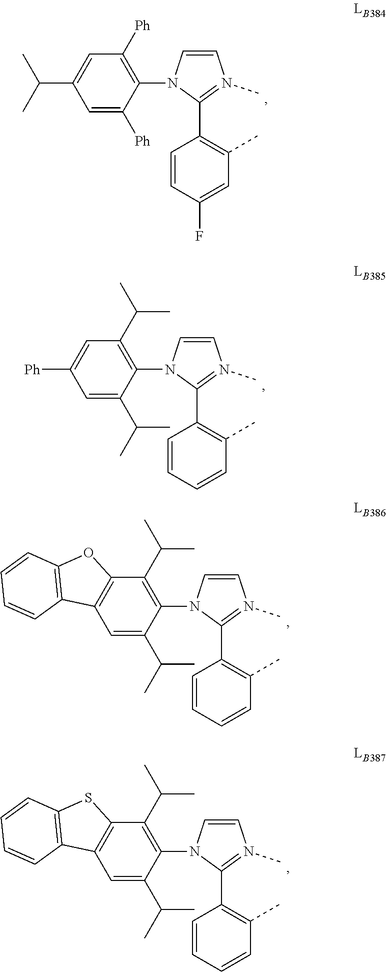

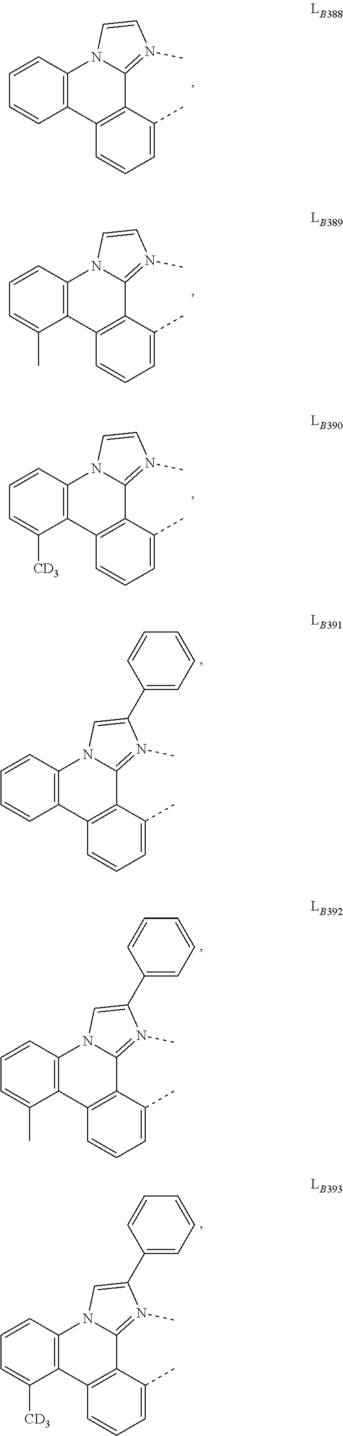

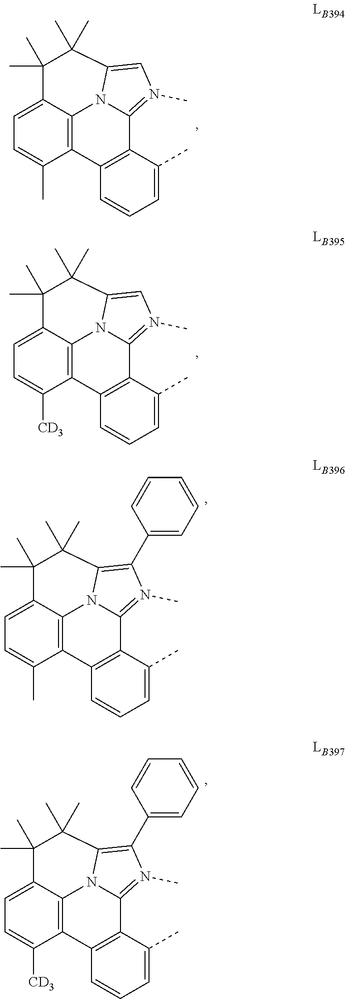

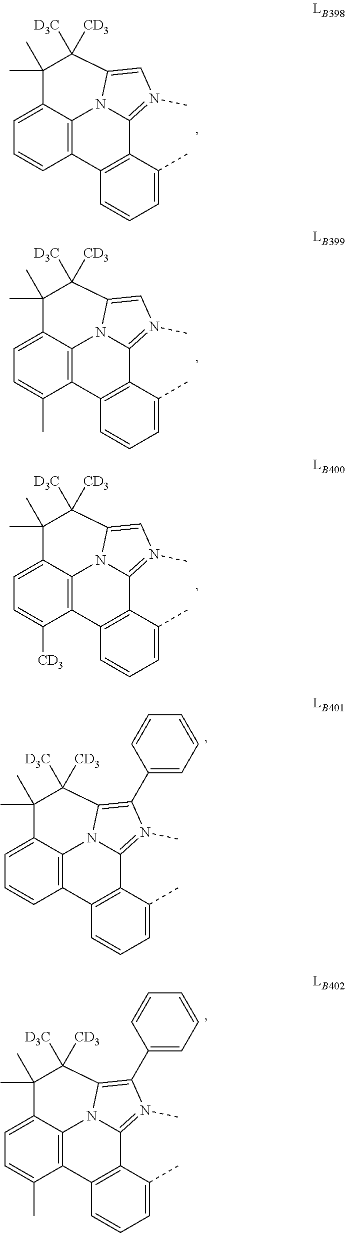

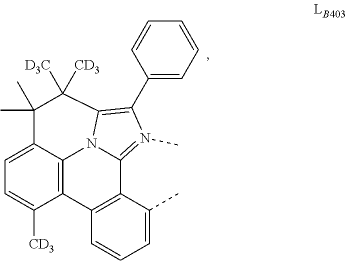

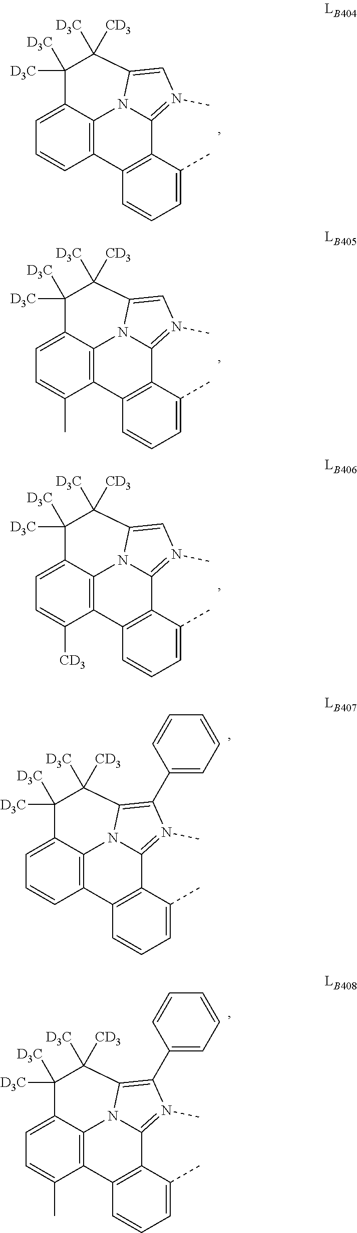

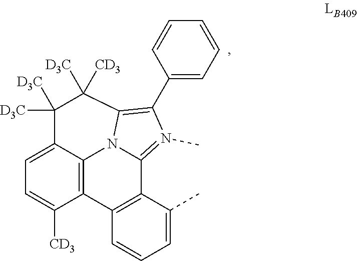

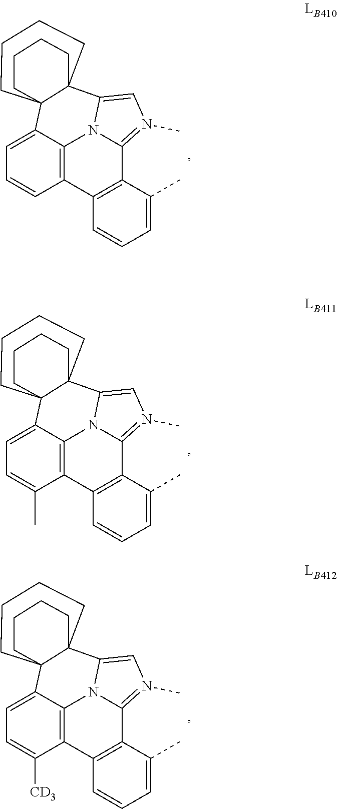

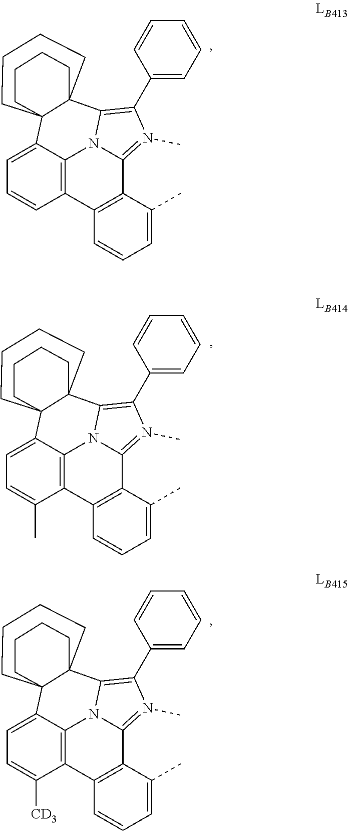

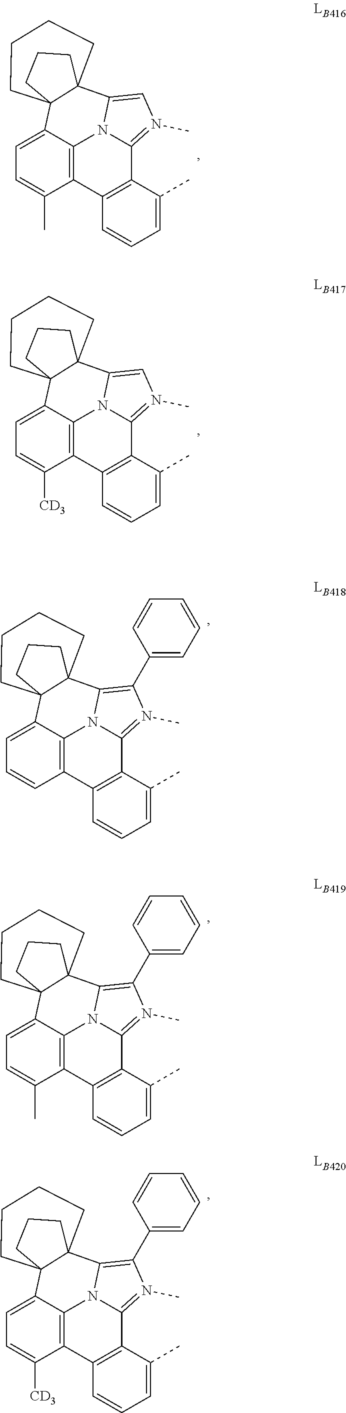

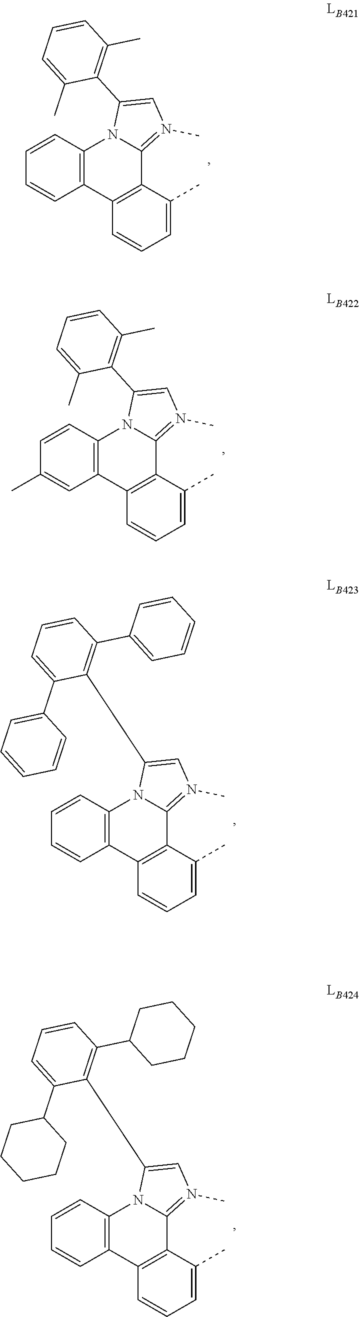

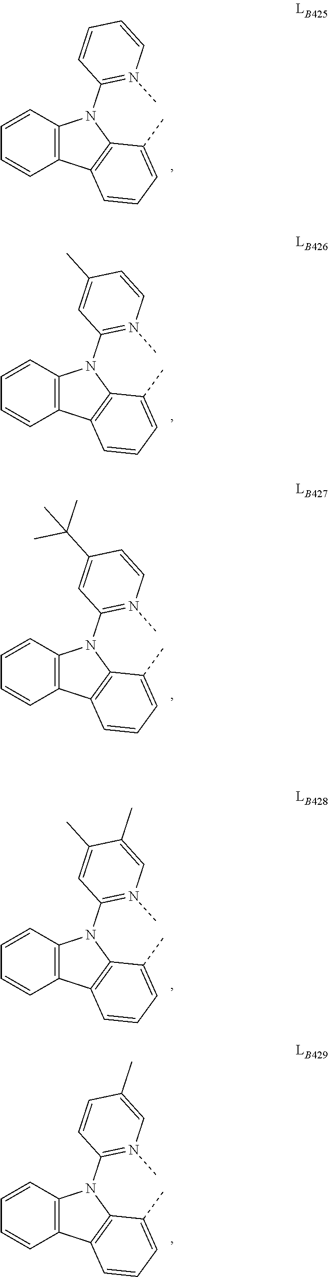

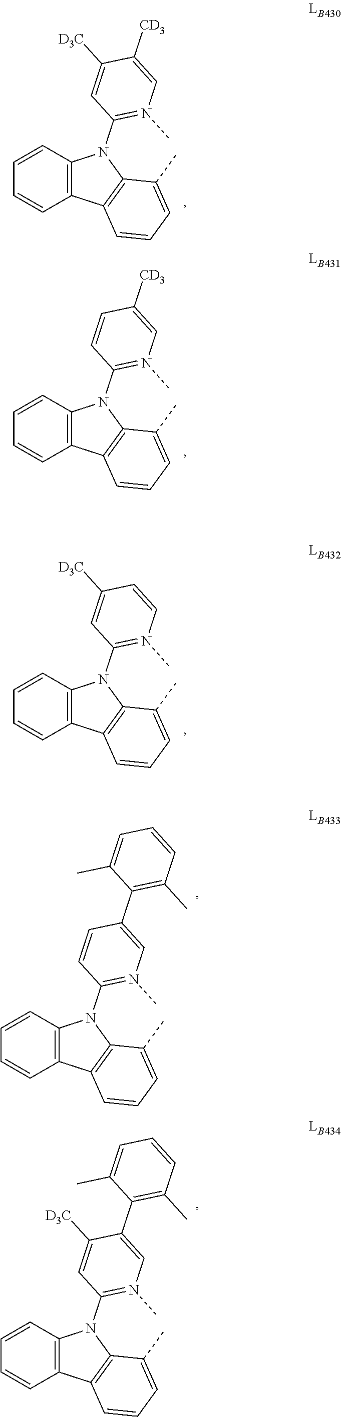

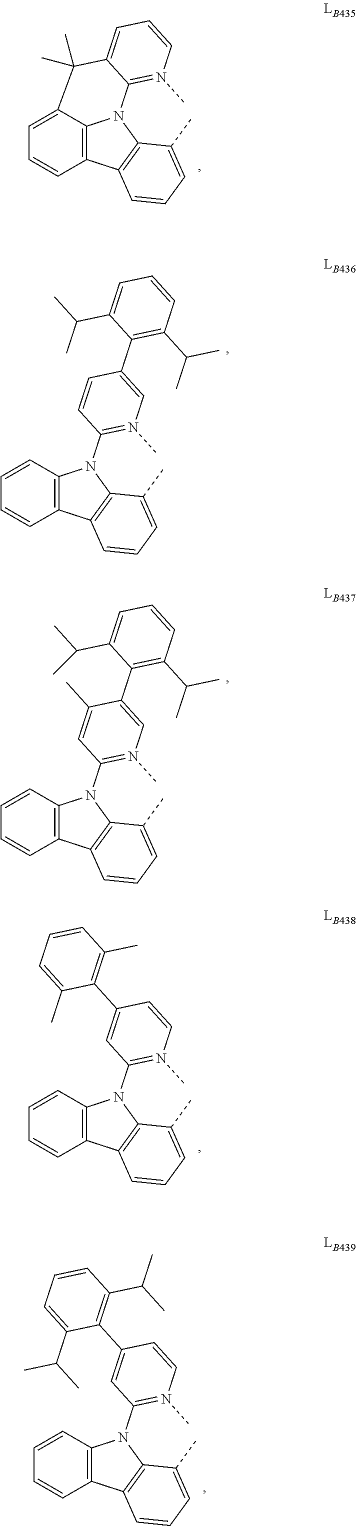

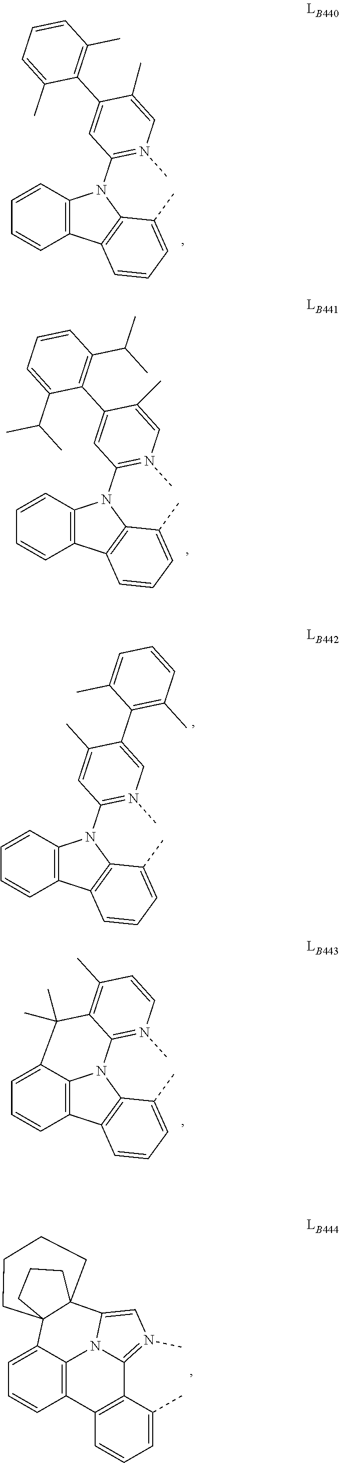

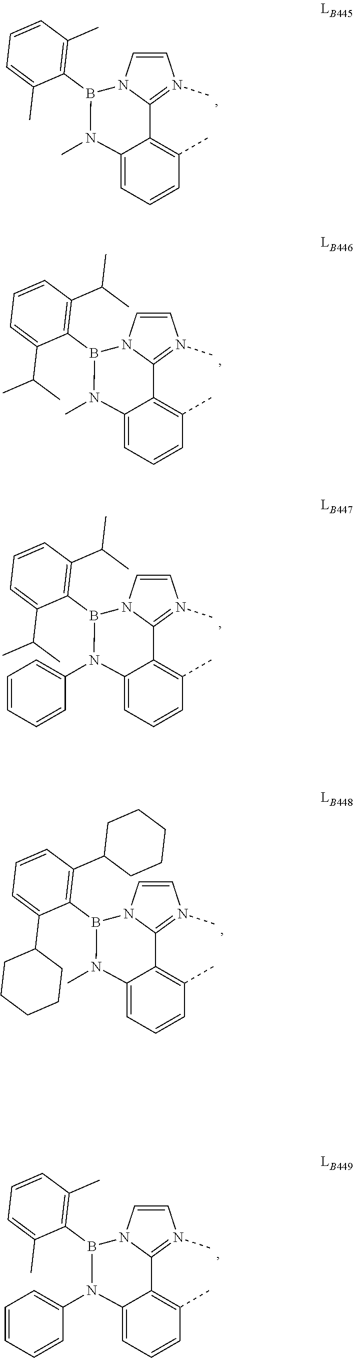

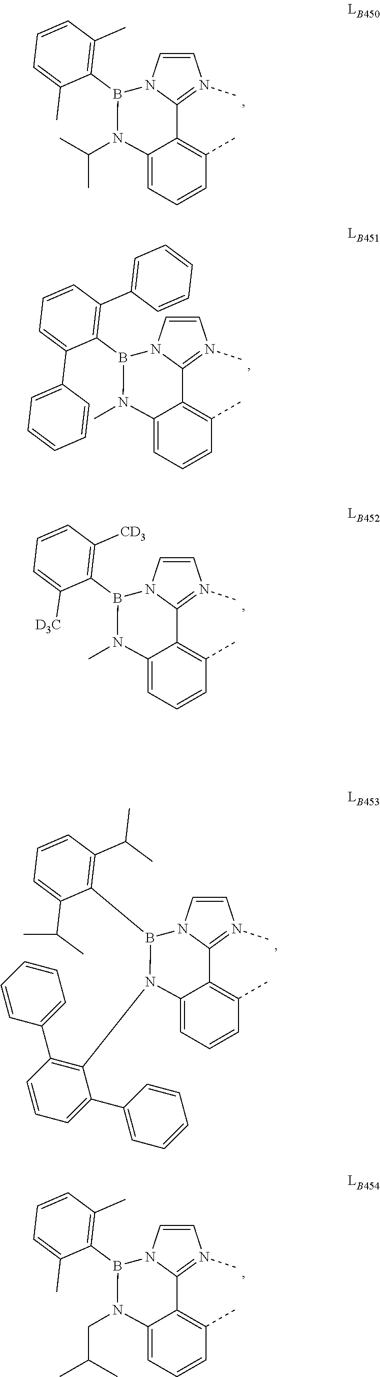

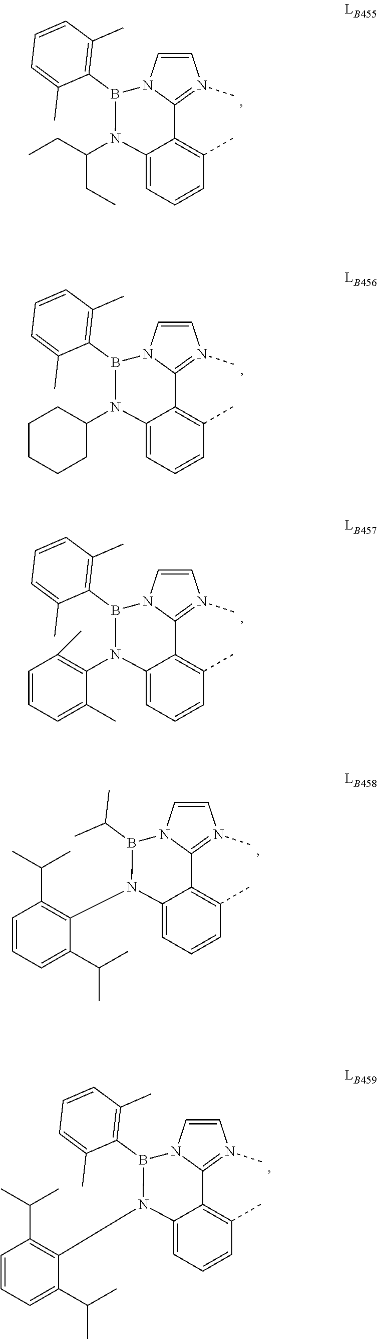

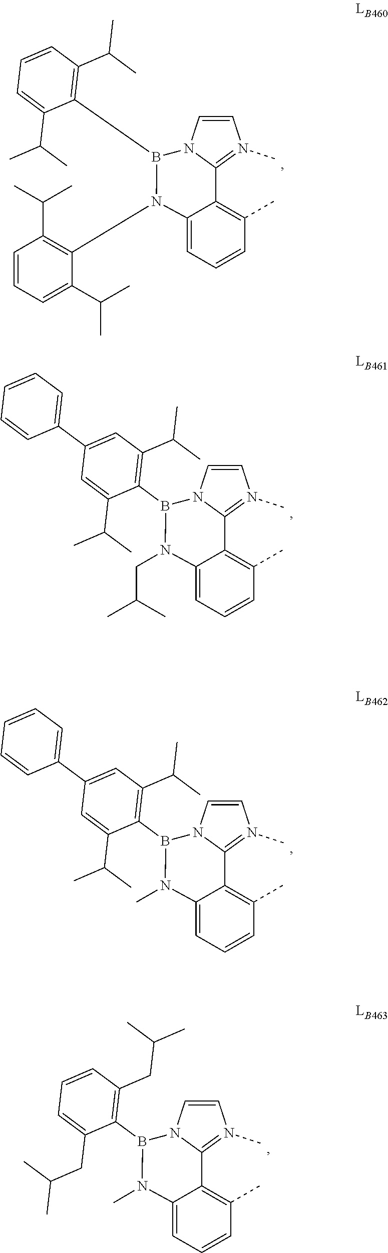

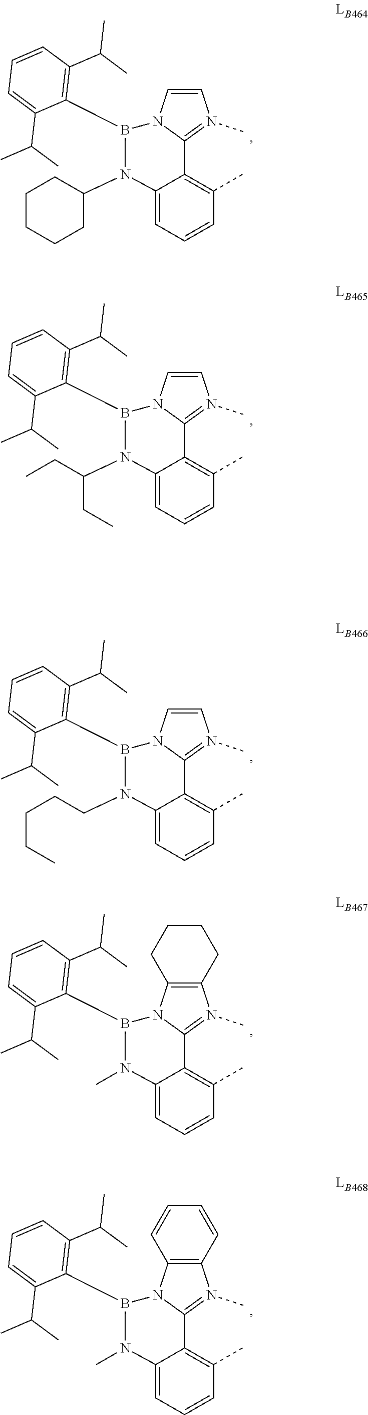

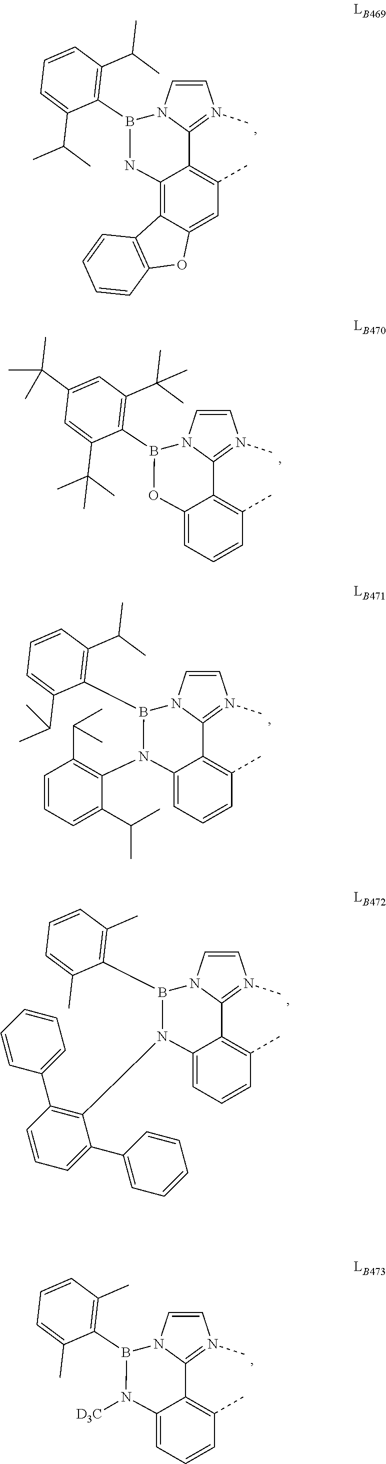

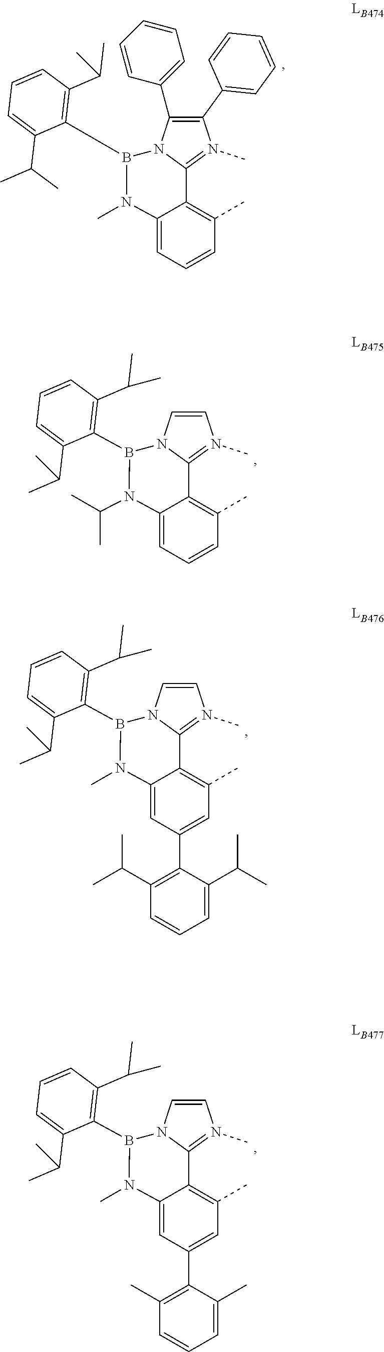

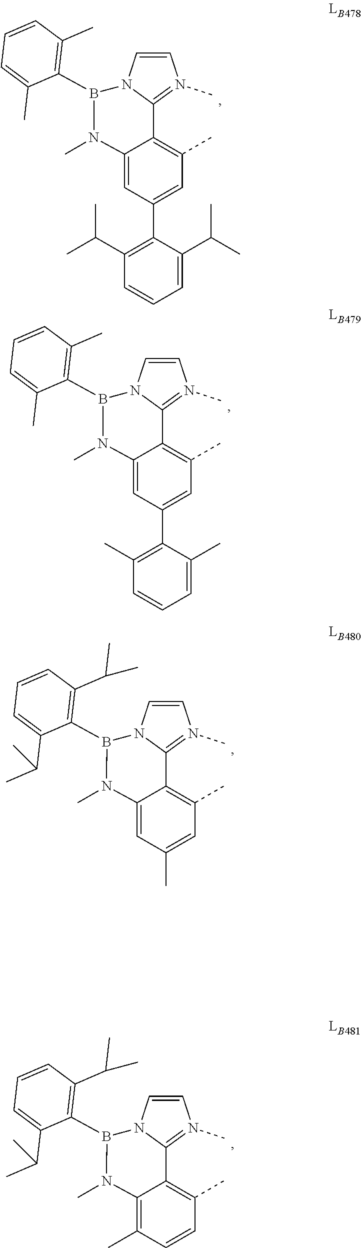

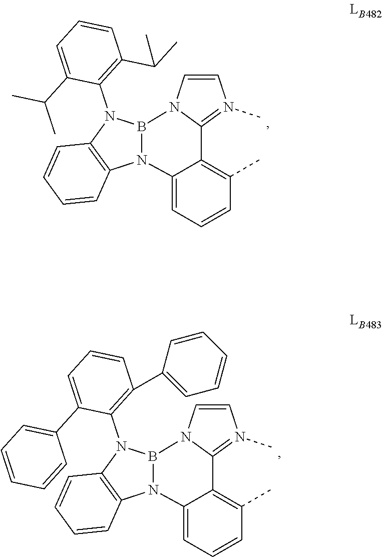

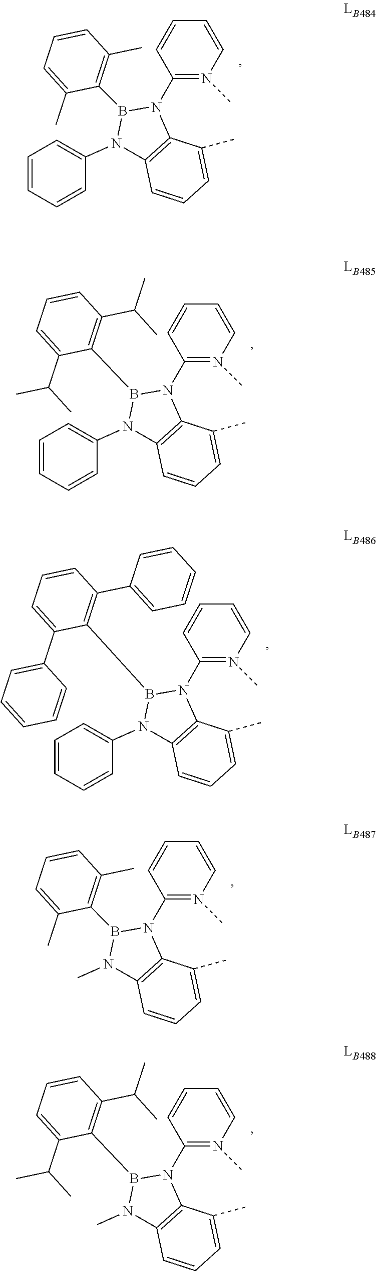

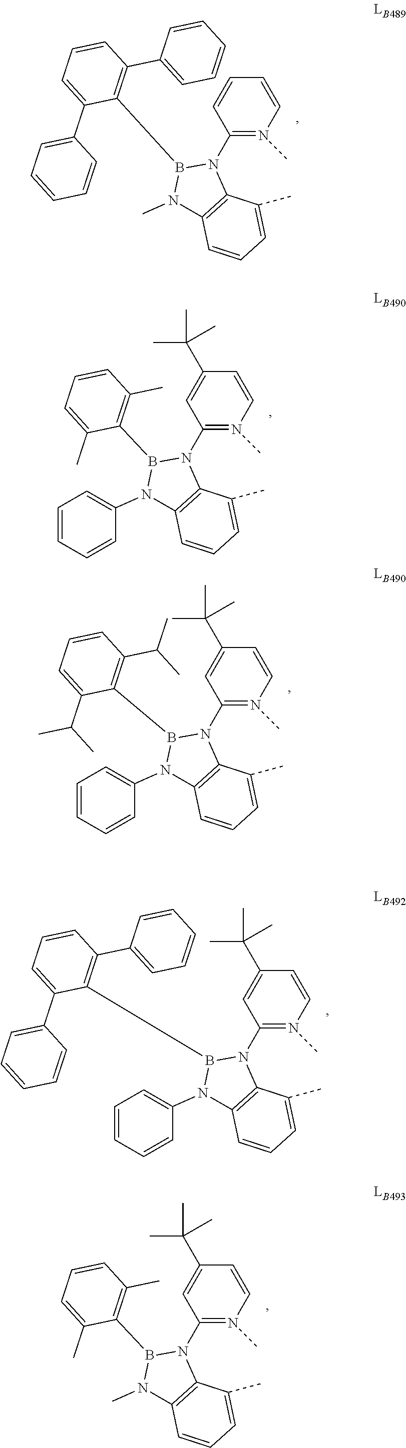

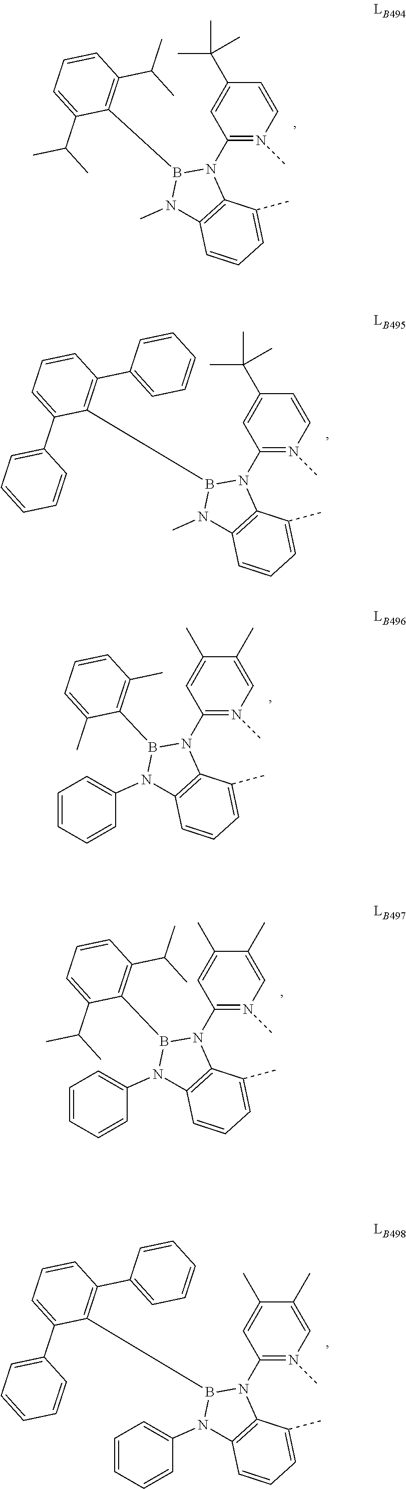

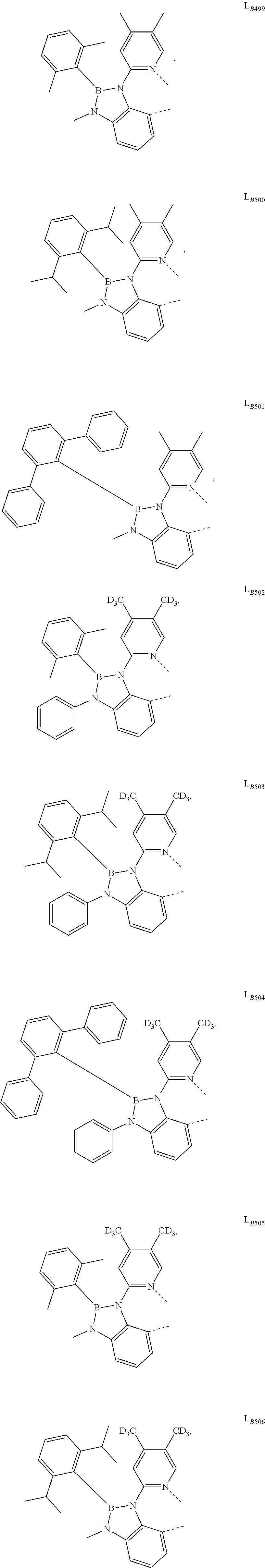

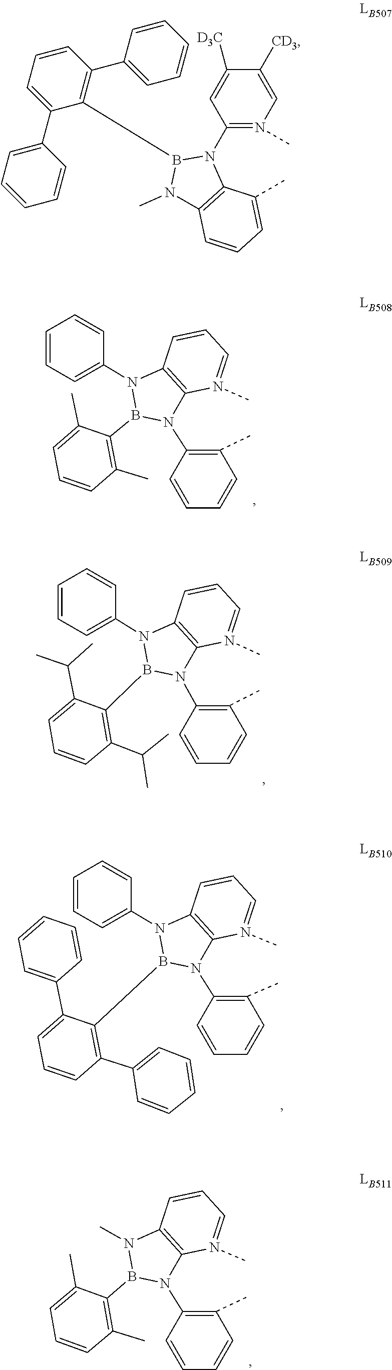

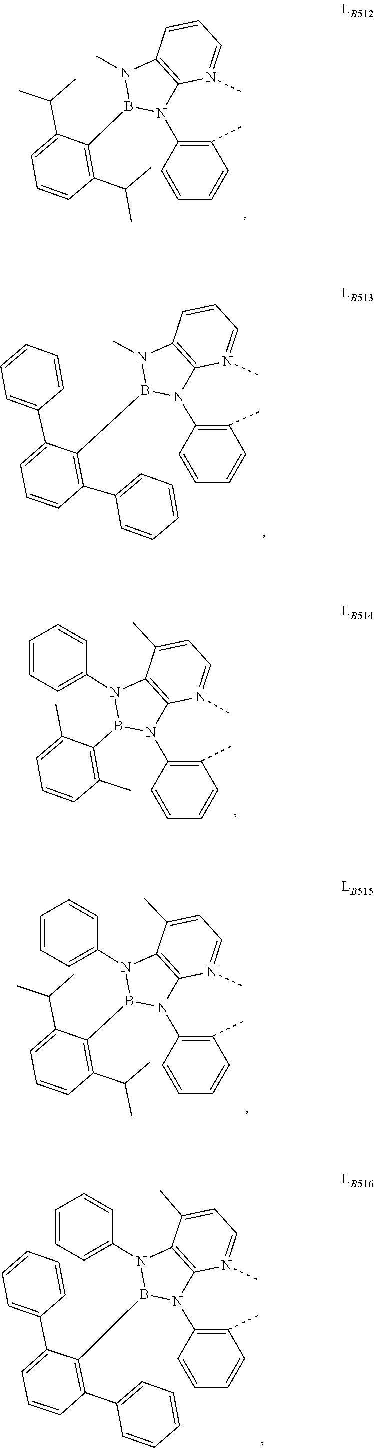

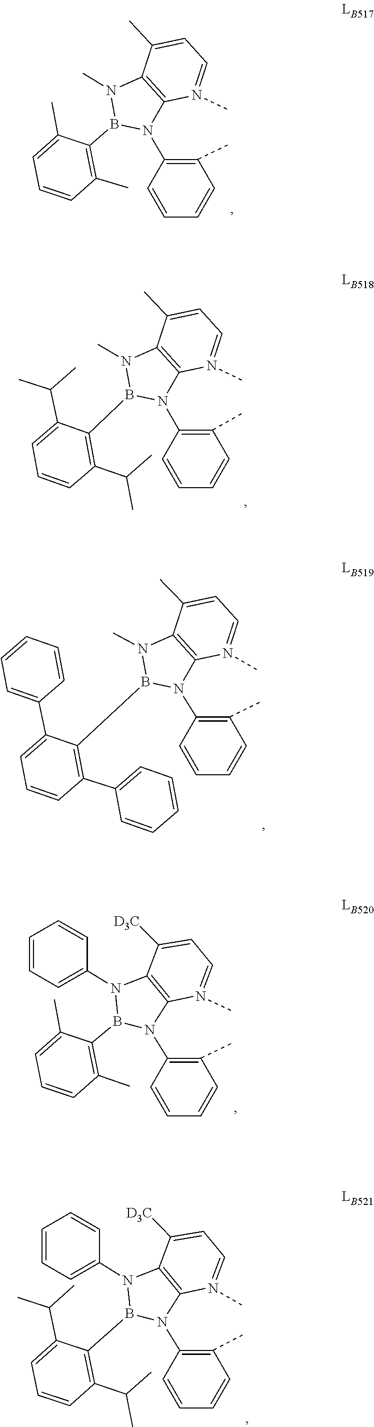

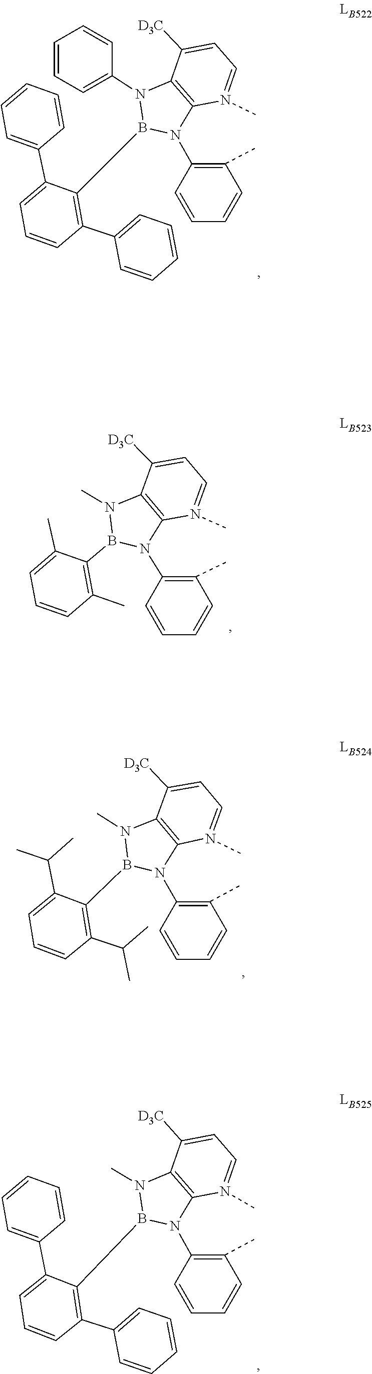

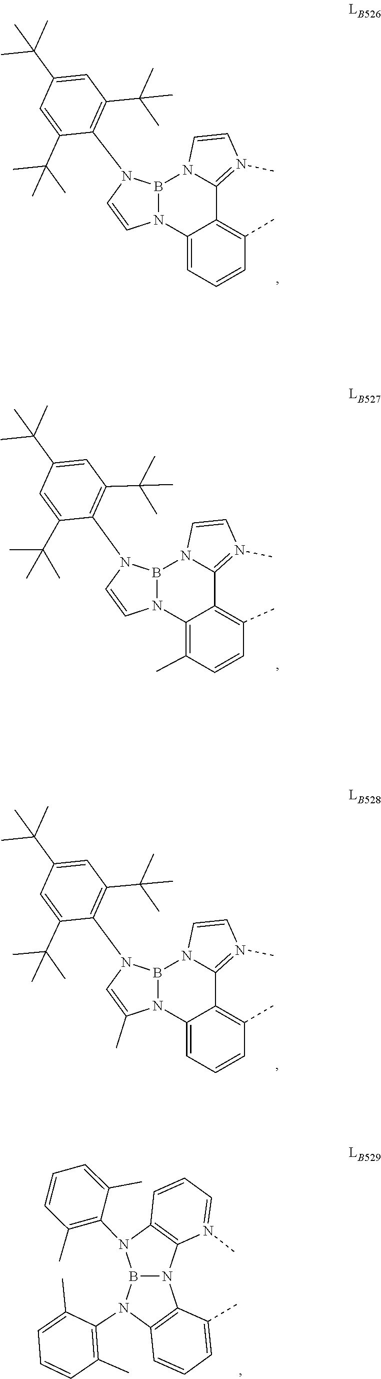

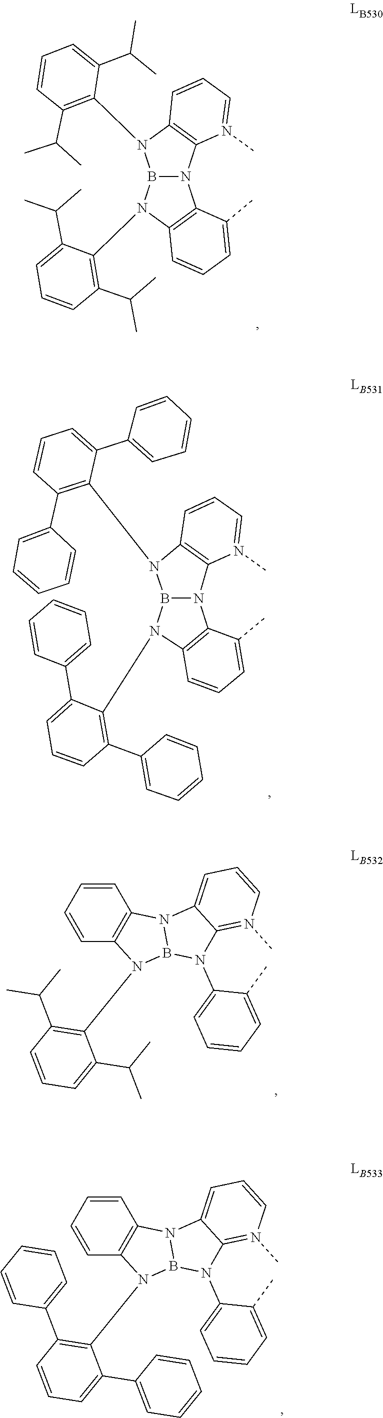

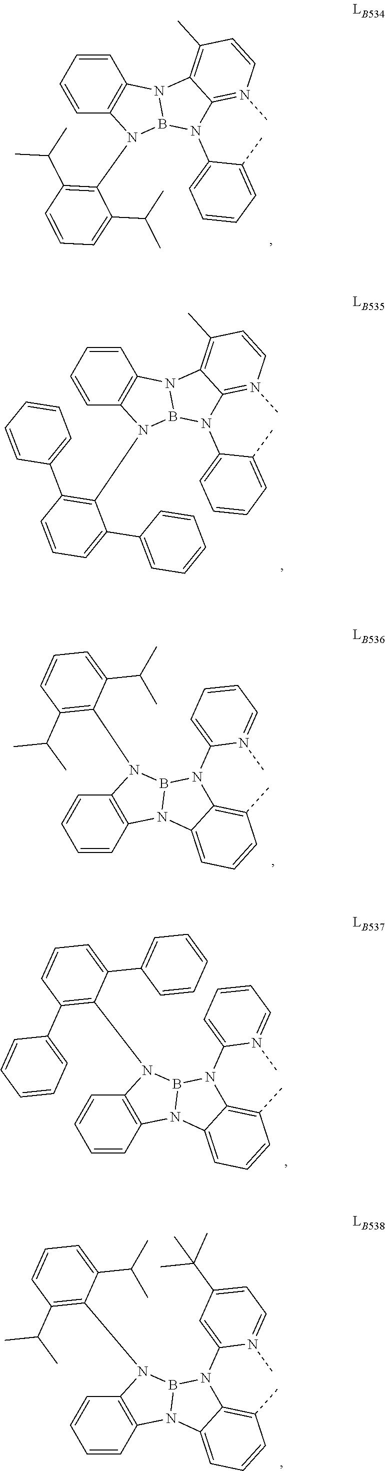

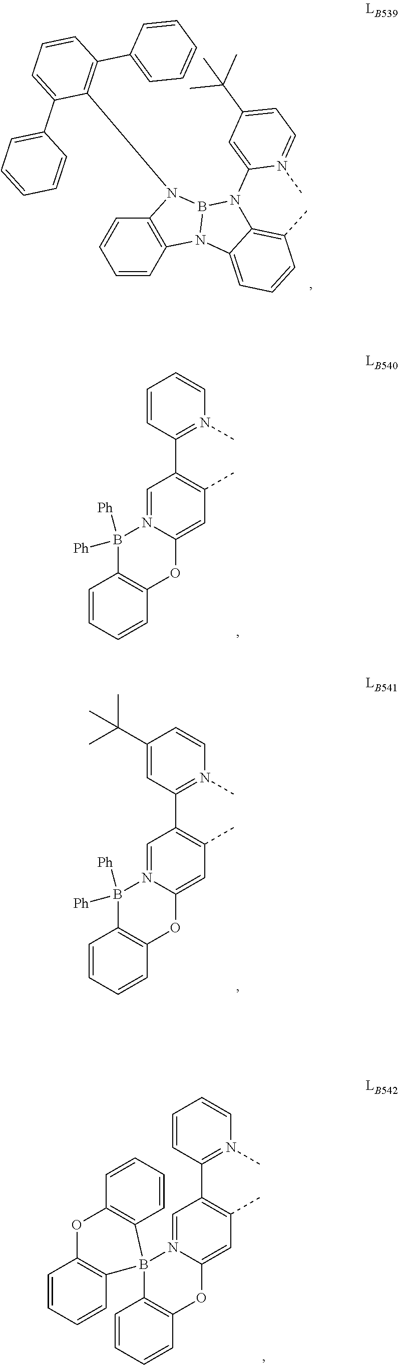

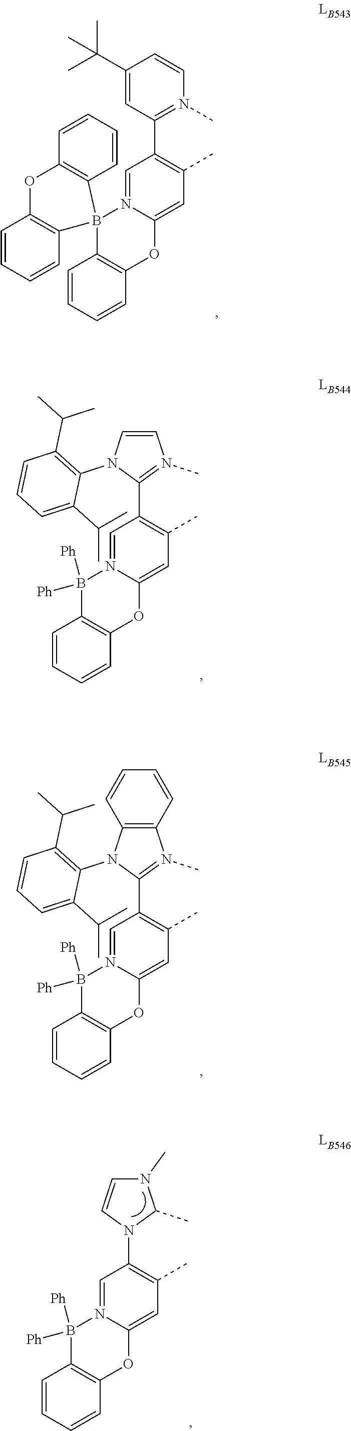

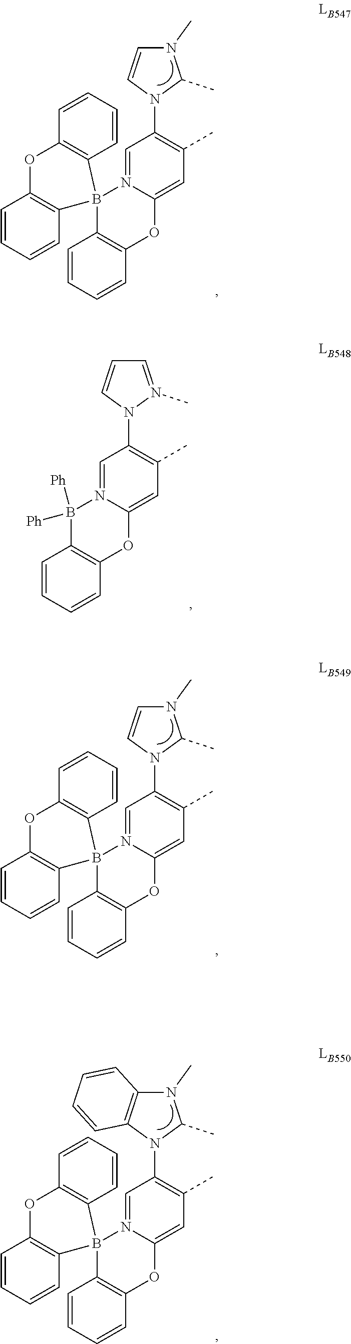

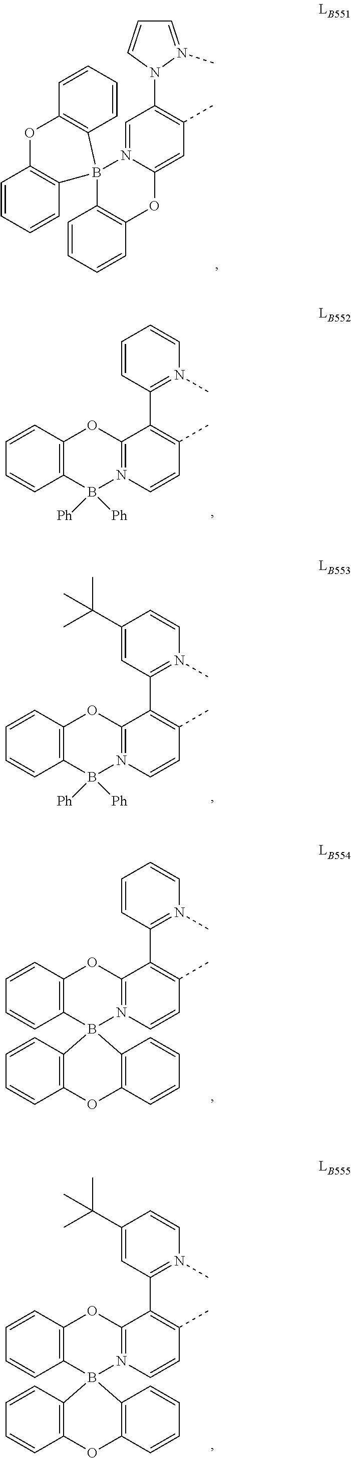

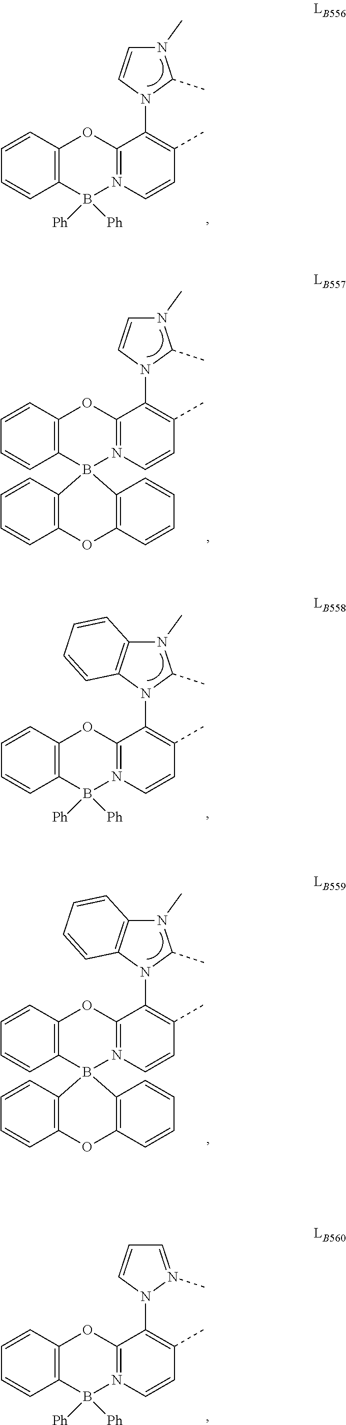

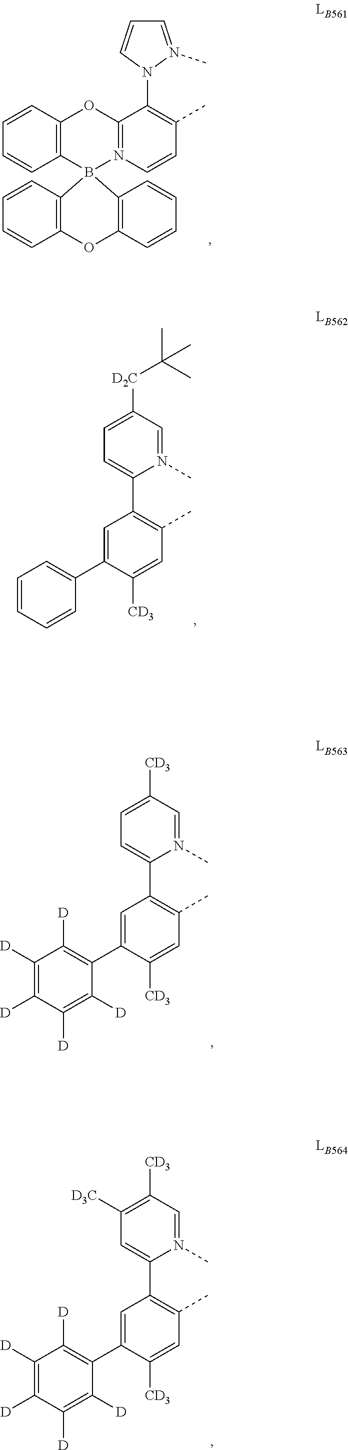

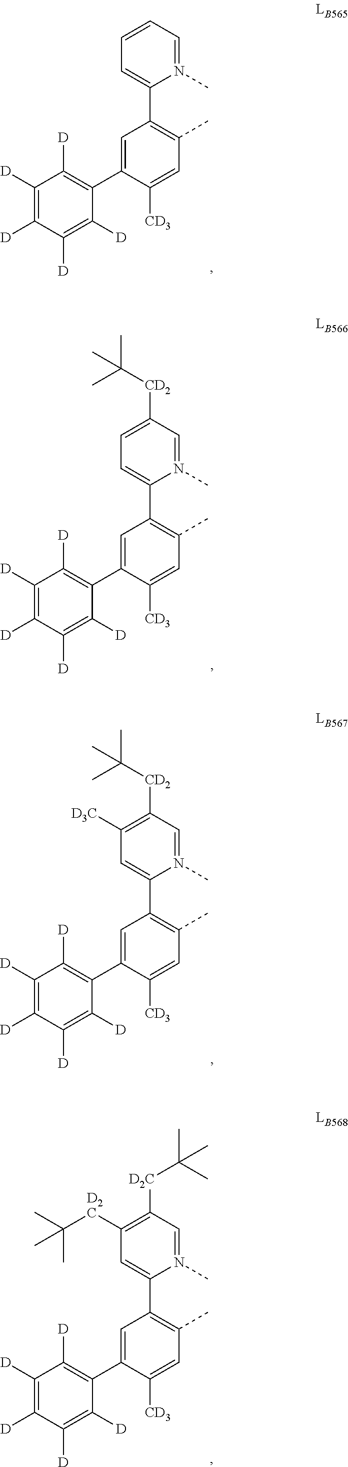

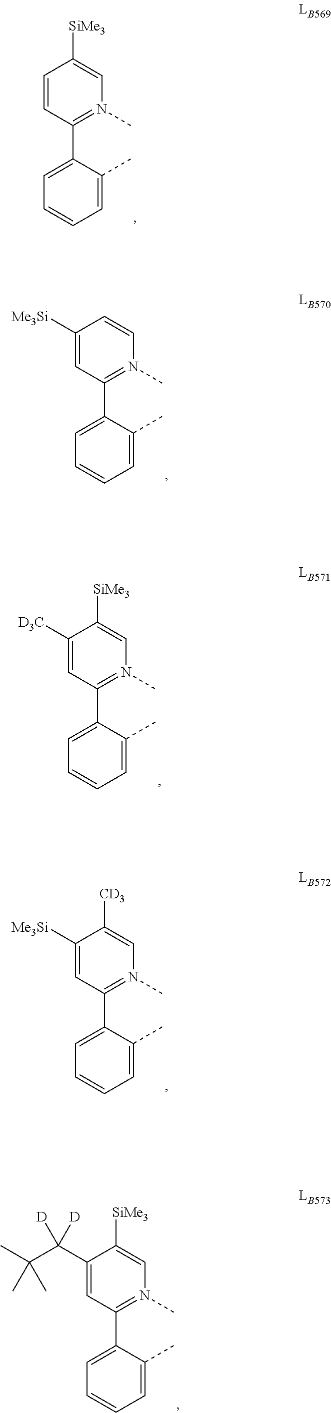

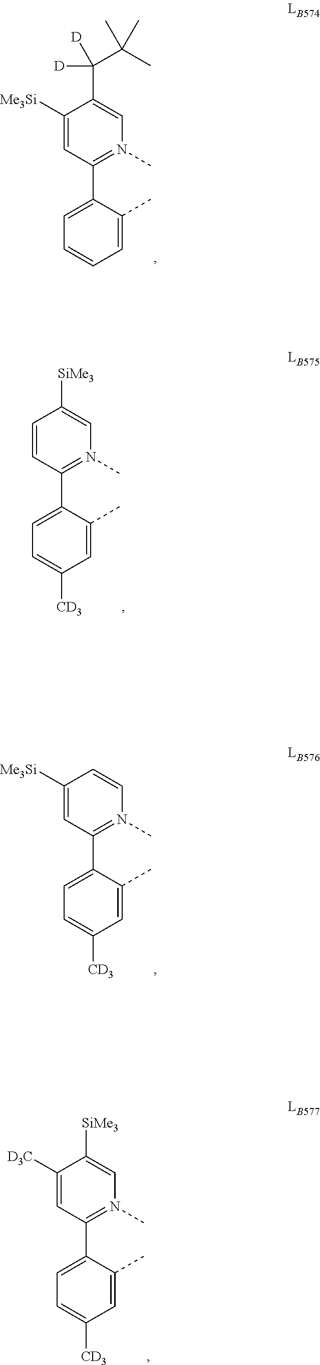

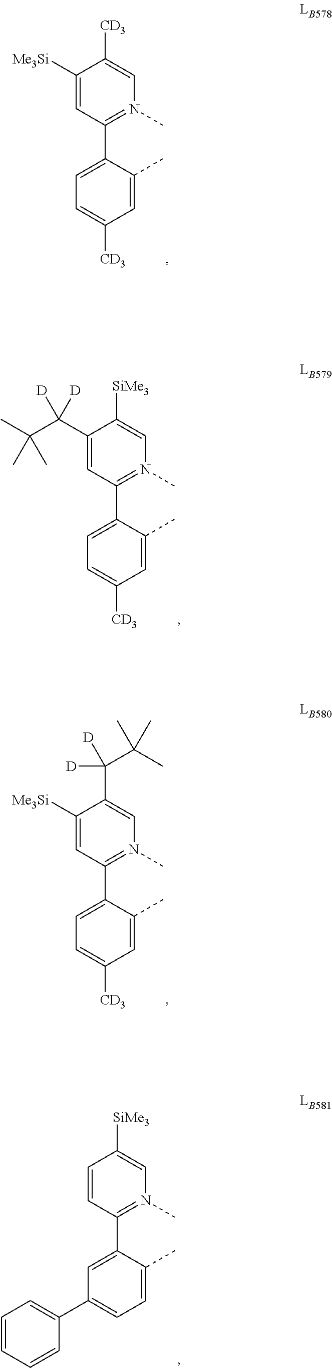

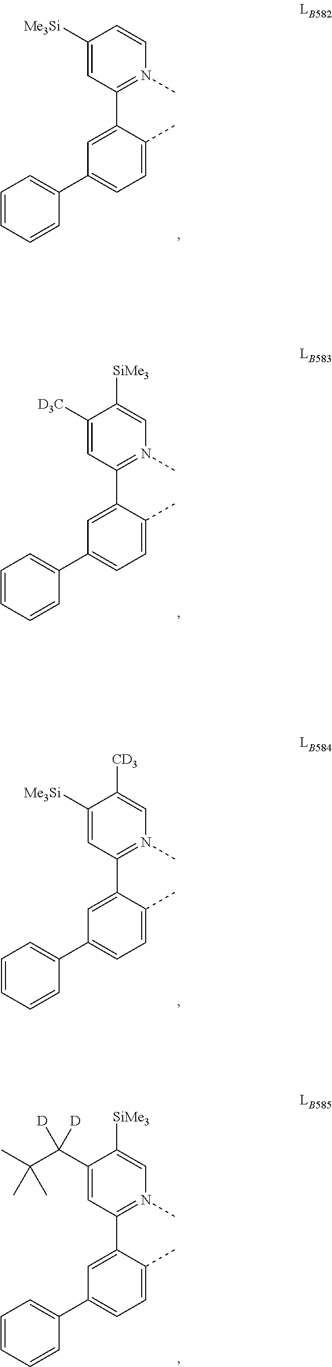

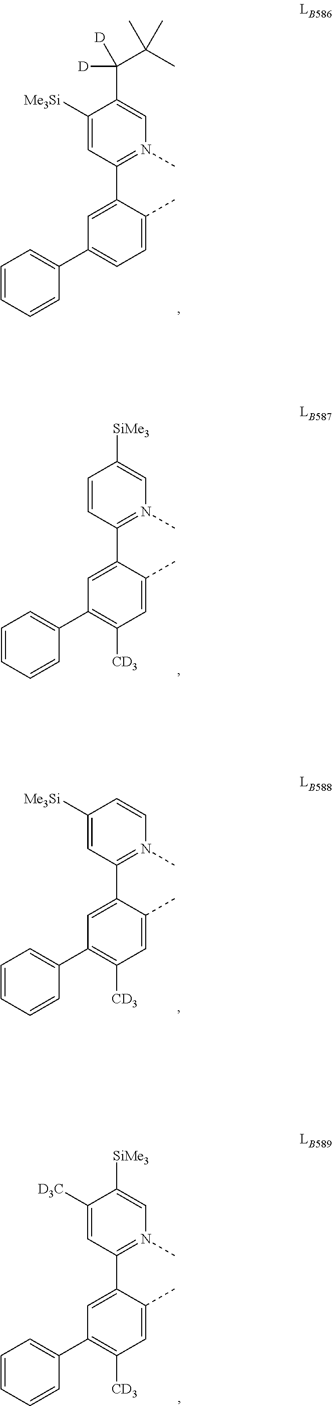

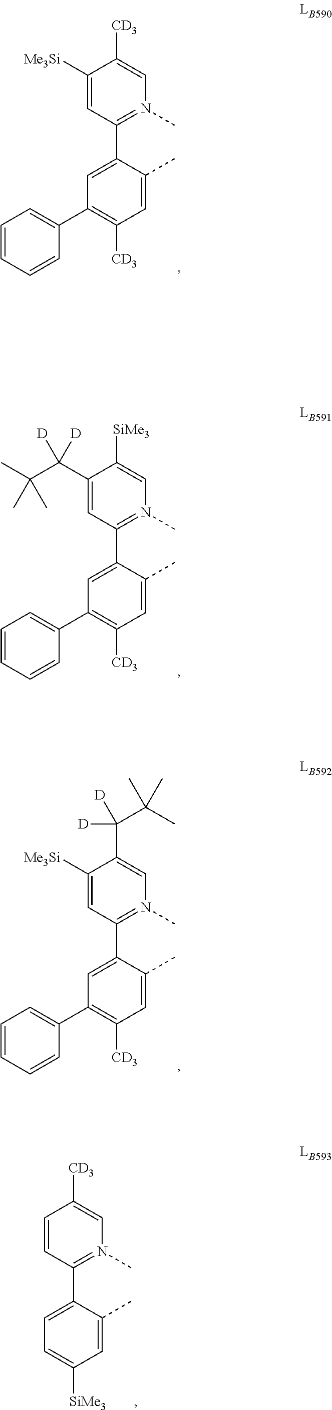

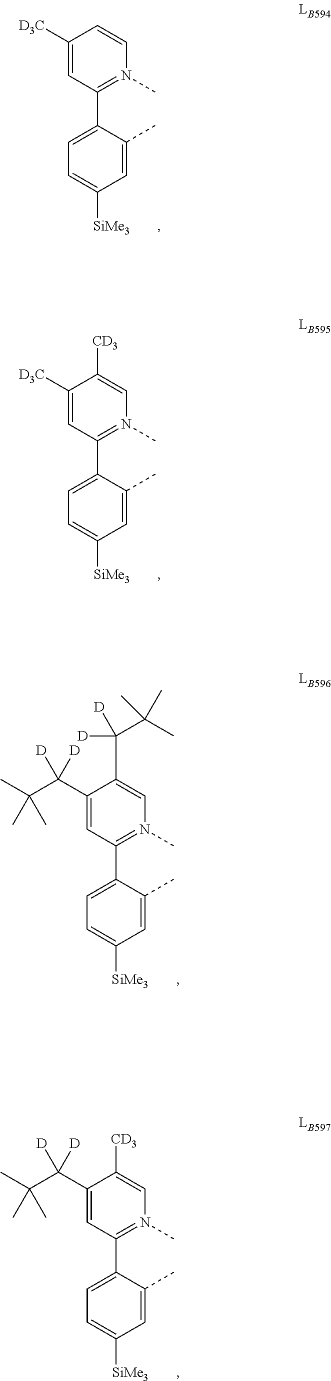

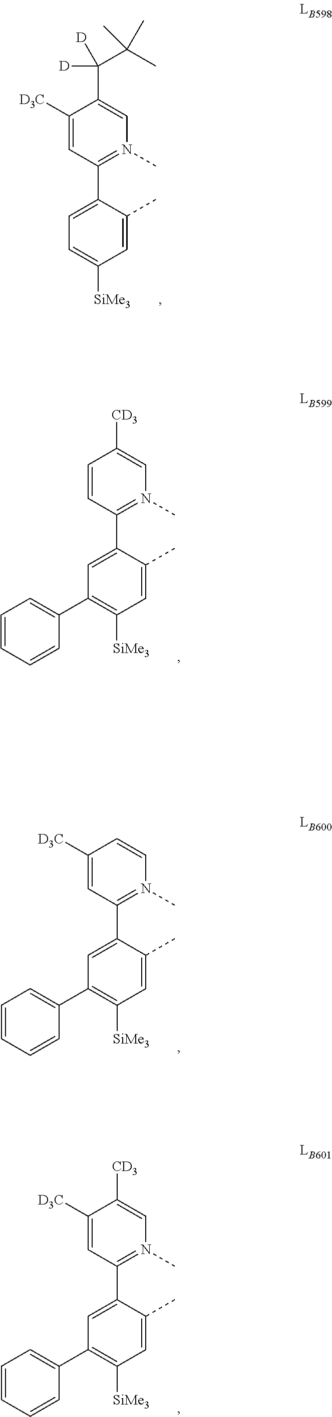

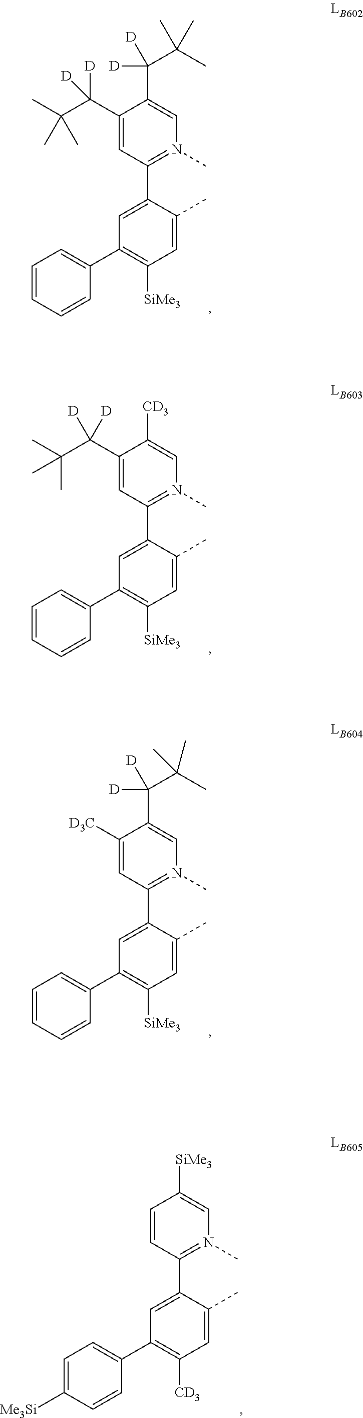

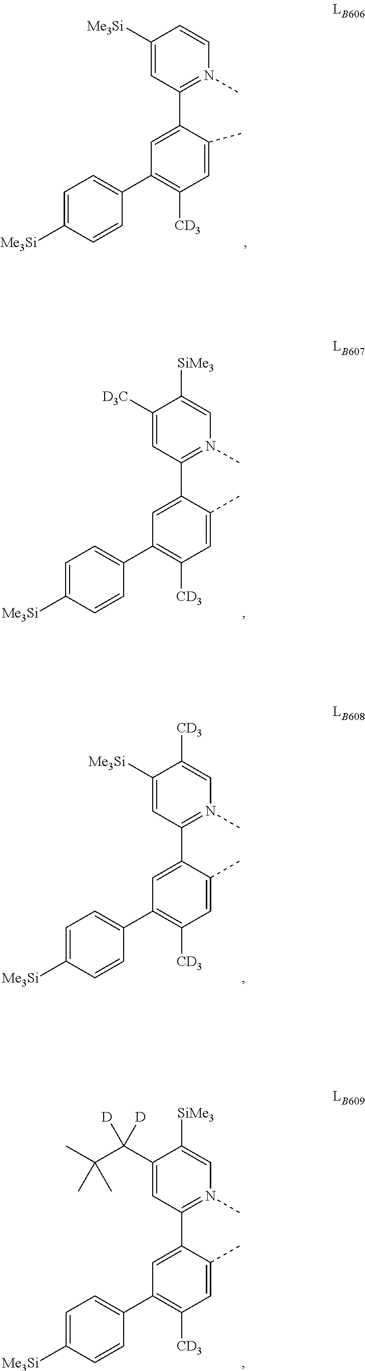

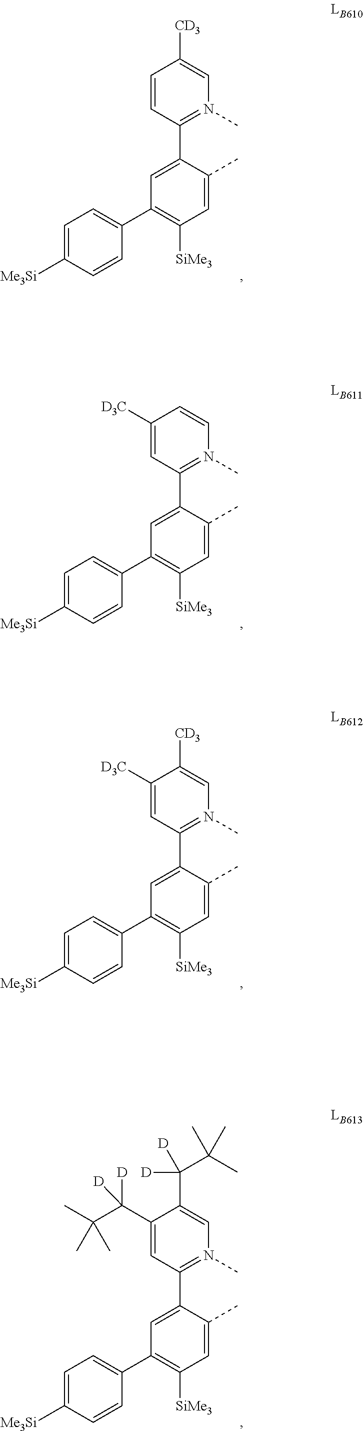

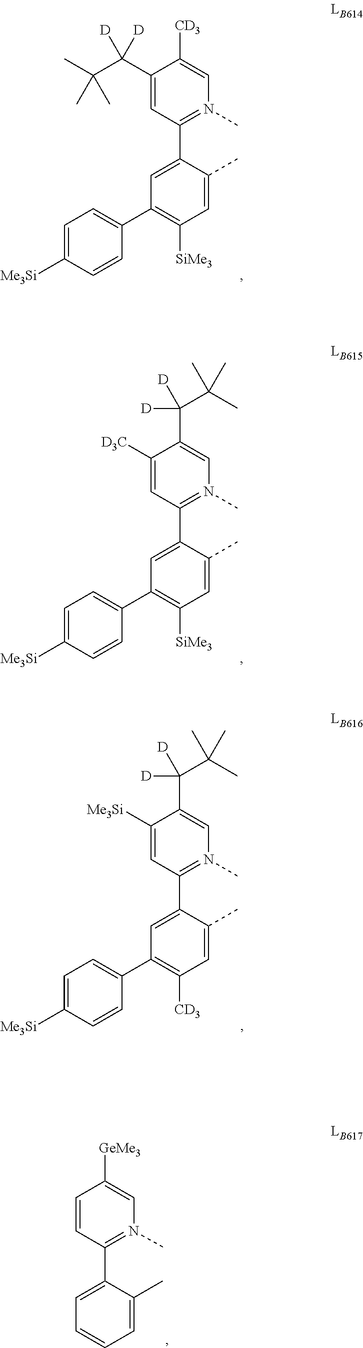

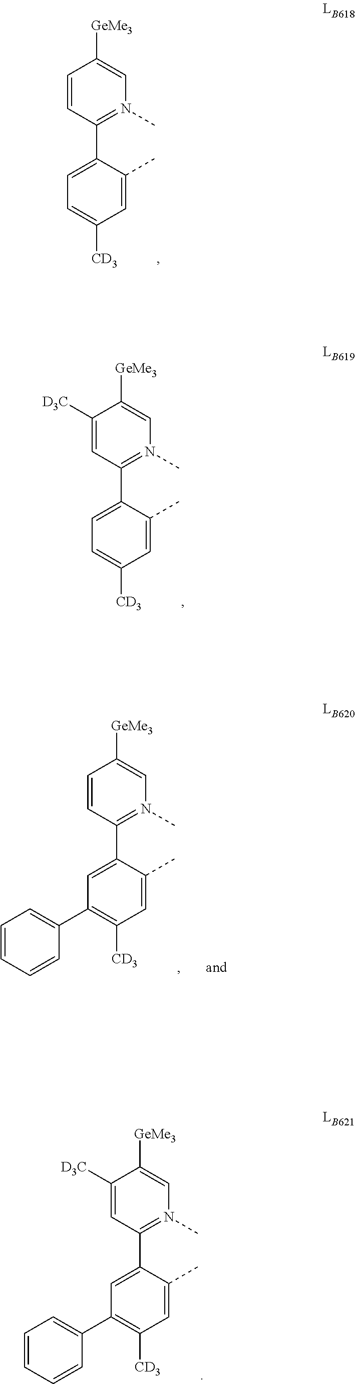

In some embodiments, the sensitizer S1 has a formula selected from the group consisting of the structures of the following LIST 9:

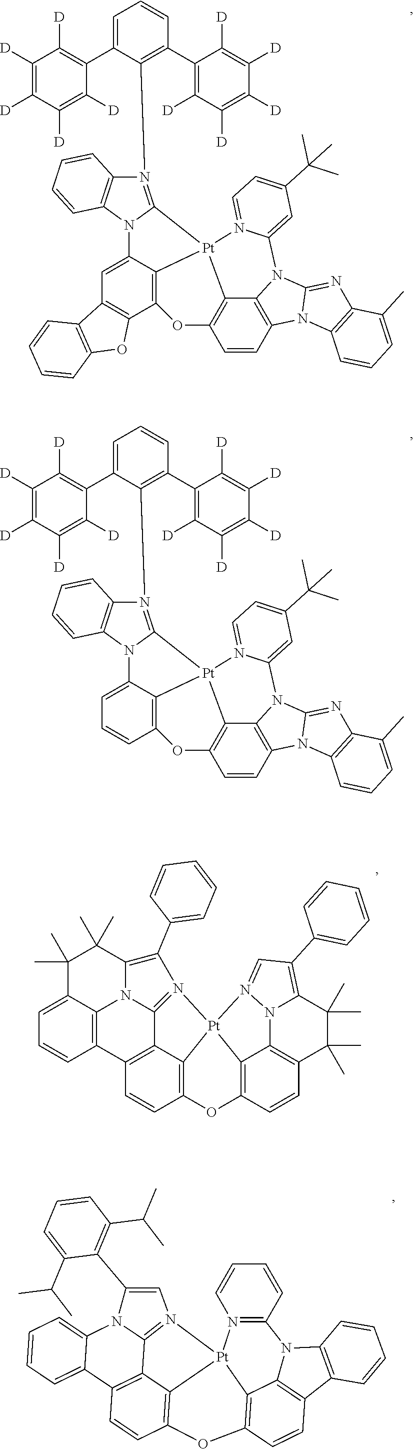

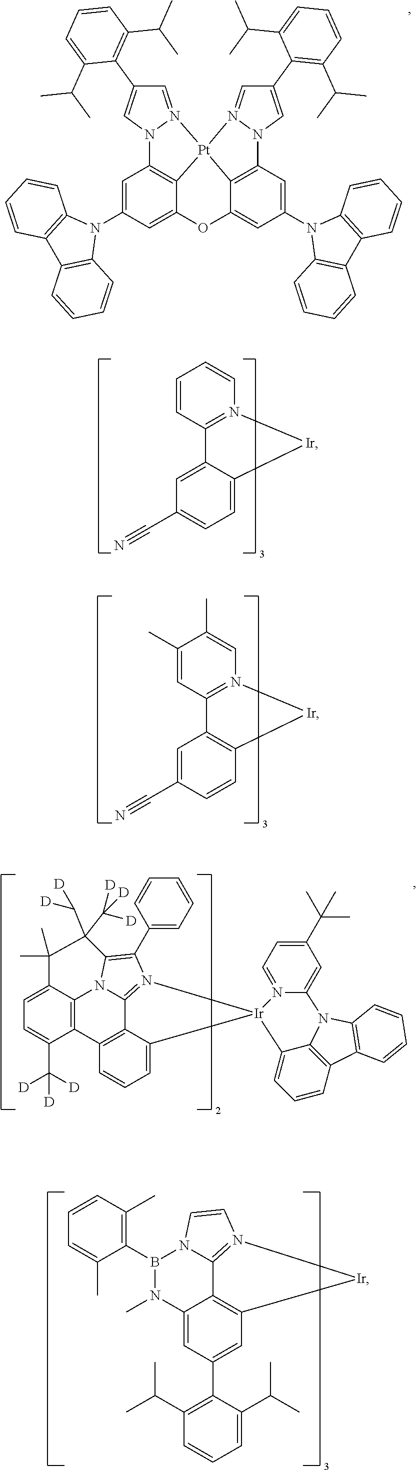

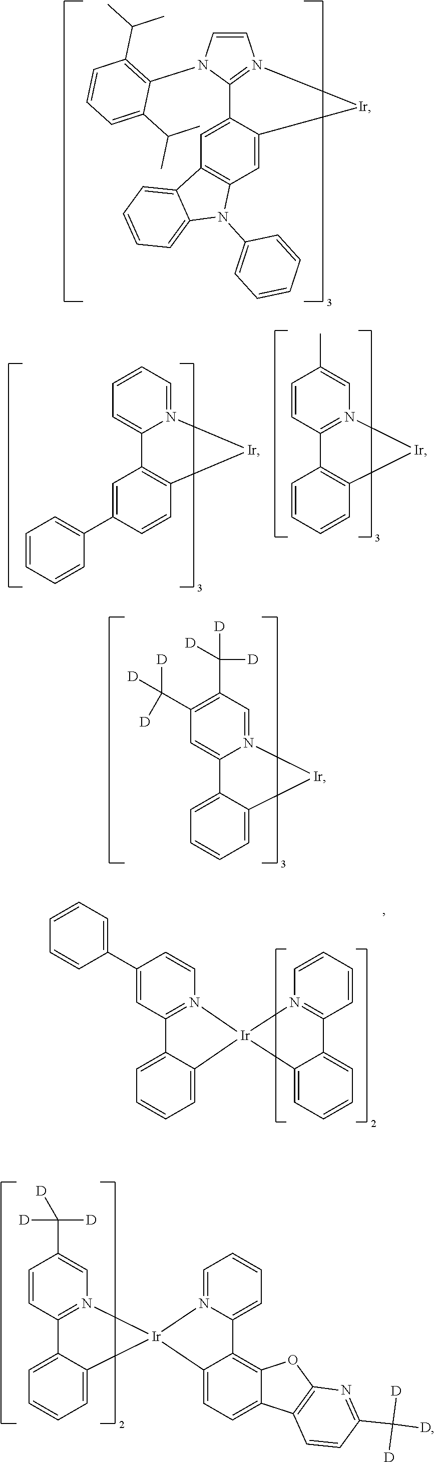

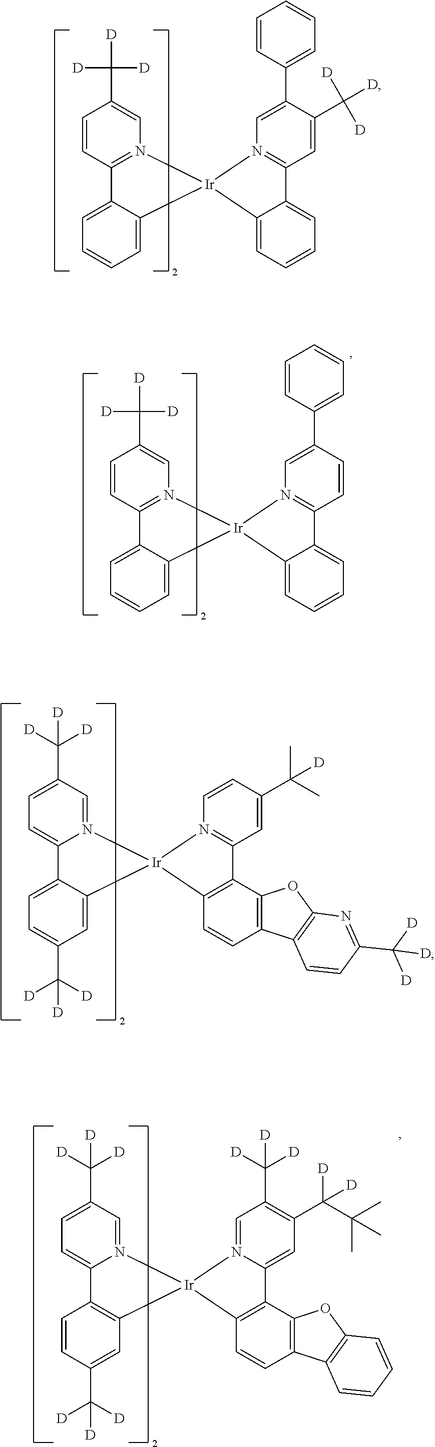

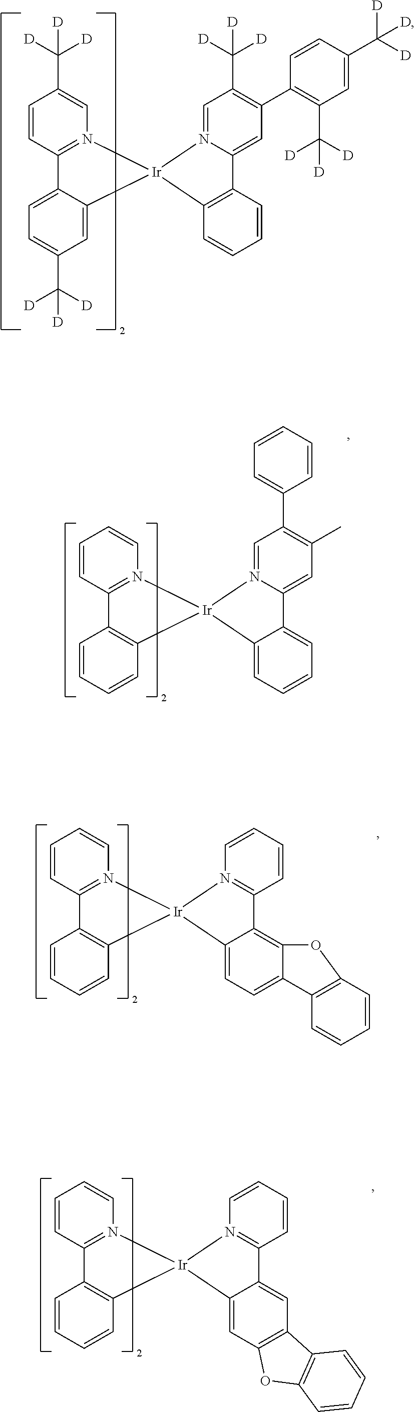

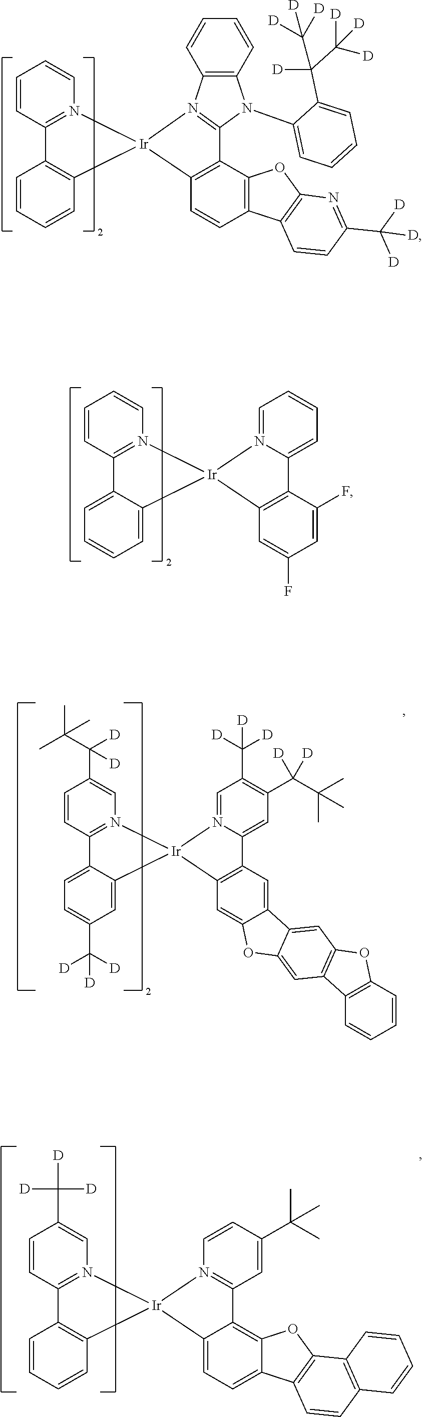

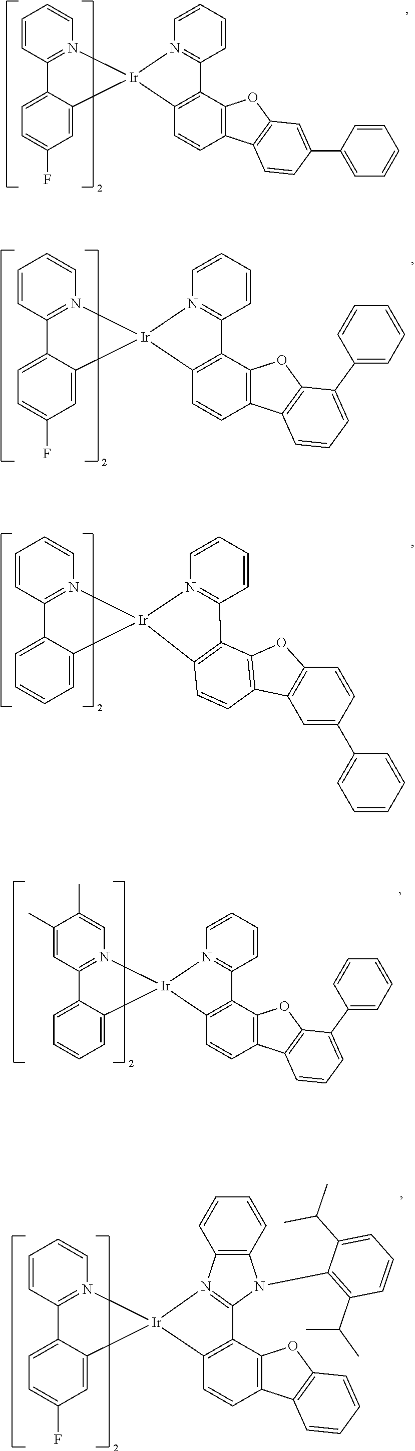

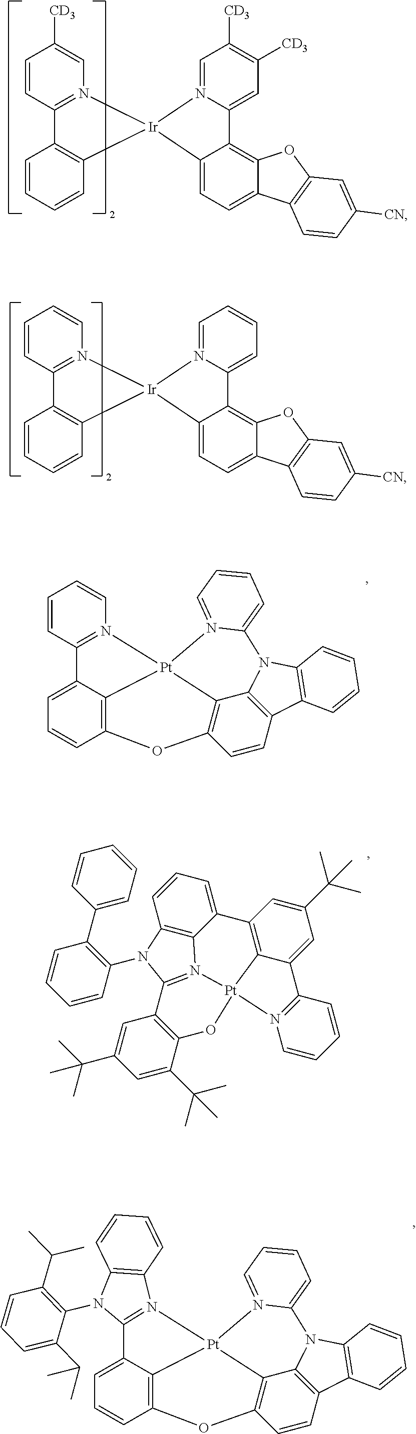

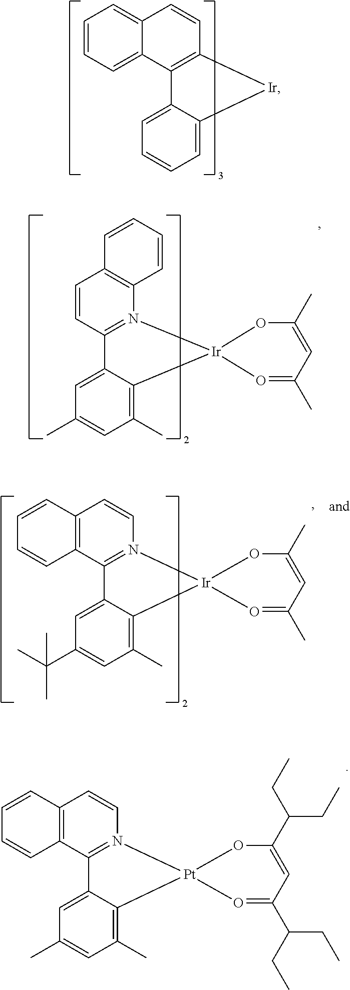

In some embodiments, the compound S1 is a fluorescent material.

In some embodiments, the compound S1 is a delayed-fluorescent compound functioning as a thermally activated delayed fluorescence (TADF) material in the OLED at room temperature.

In some embodiments, the TADF material may be as described below.

In some embodiments, the TADF material comprises at least one donor group and at least one acceptor group.

In some embodiments, the TADF material is a metal complex.

In some embodiments, the TADF material is a Cu, Ag, or Au complex.

In some embodiments, the TADF material is a non-metal complex.

In some embodiments, the TADF material has the formula of M(L⁵)(L⁶);
wherein M is Cu, Ag, or Au;
wherein L⁵ and L⁶ are different, and
wherein each of L⁵ and L⁶ are independently selected from the group consisting of the structures of the following LIST 11 defined herein:

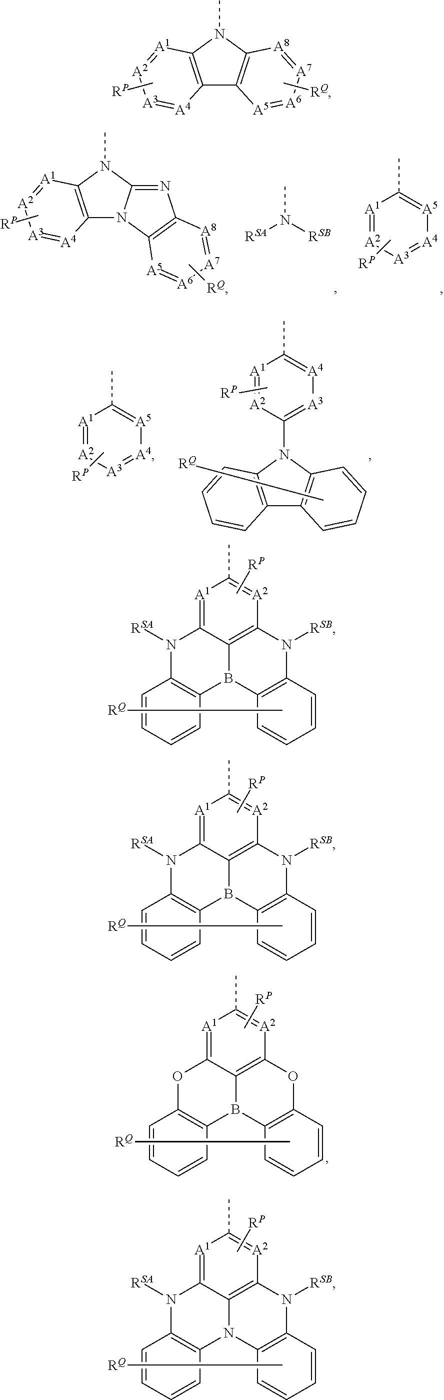

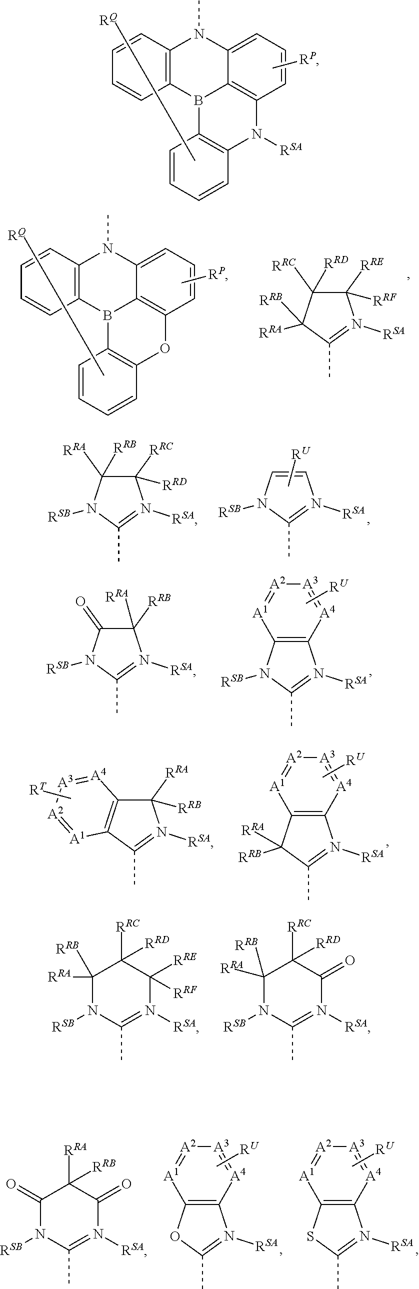

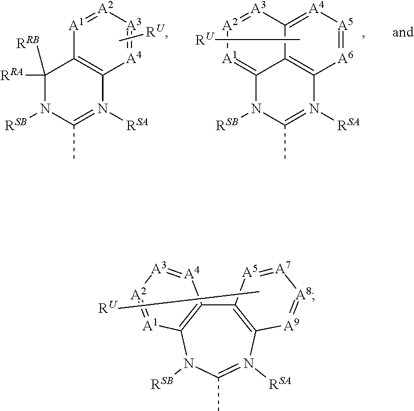

wherein each of A¹ to A⁹ is independently selected from C or N;
each of R^{P}, R^{Q}, R^{T}, and R^{U} independently represents mono-, up to the maximum substitutions, or no substitutions;
wherein each R^{P}, R^{Q}, R^{T}, R^{U}, R^{SA}, R^{SB}, R^{RA}, R^{RB}, R^{RC}, R^{RD}, R^{RE}, and R^{RF} is independently a hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof; and
wherein any two substituents can be joined or fused to form a ring.

In some embodiments, the TADF material is selected from the group consisting of the structures of the following LIST 12 defined herein:

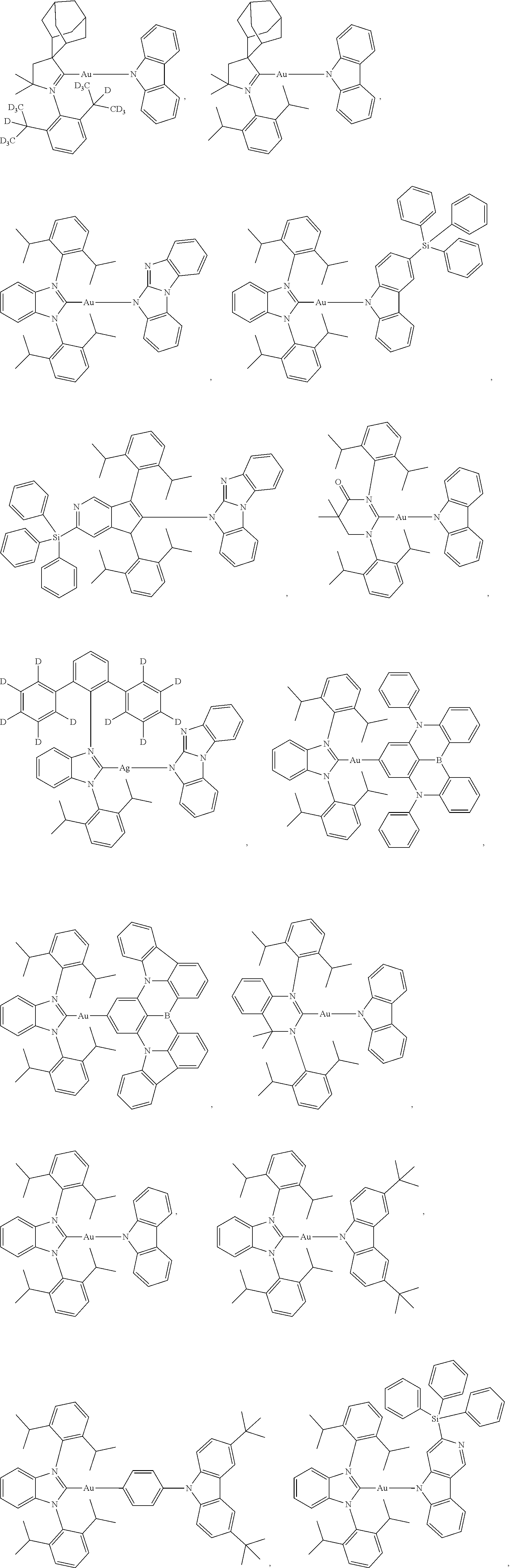

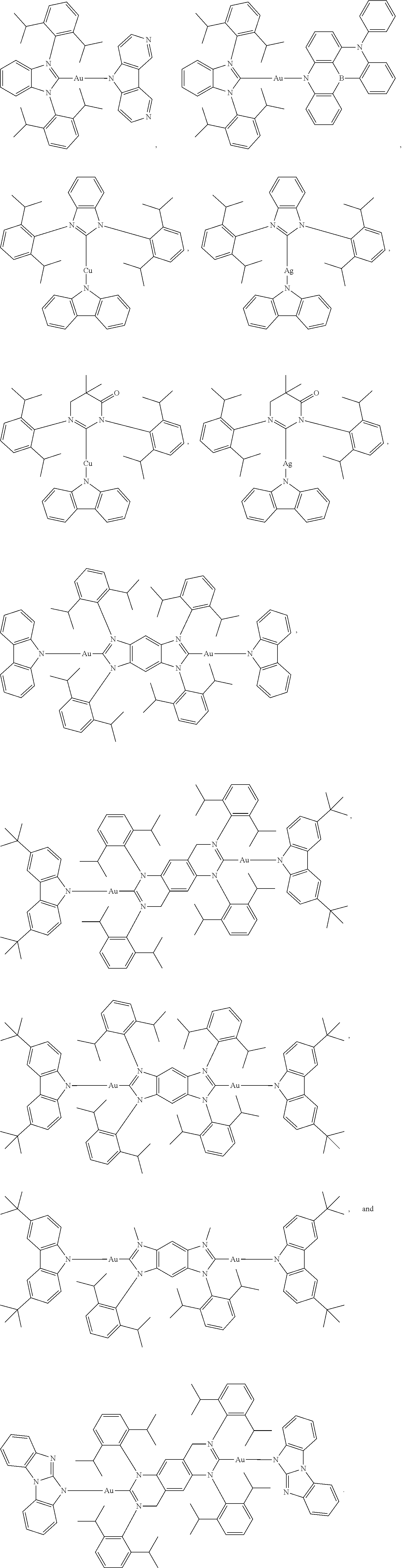

In some embodiments, the TADF material comprises at least one of the chemical moieties selected from the group consisting of the structures of the following LIST 14 defined herein:

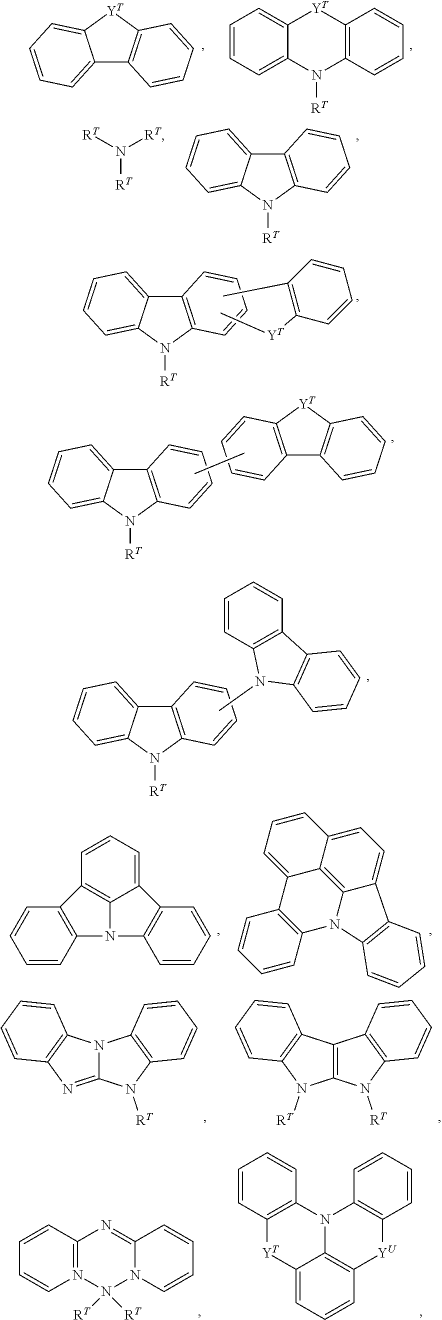

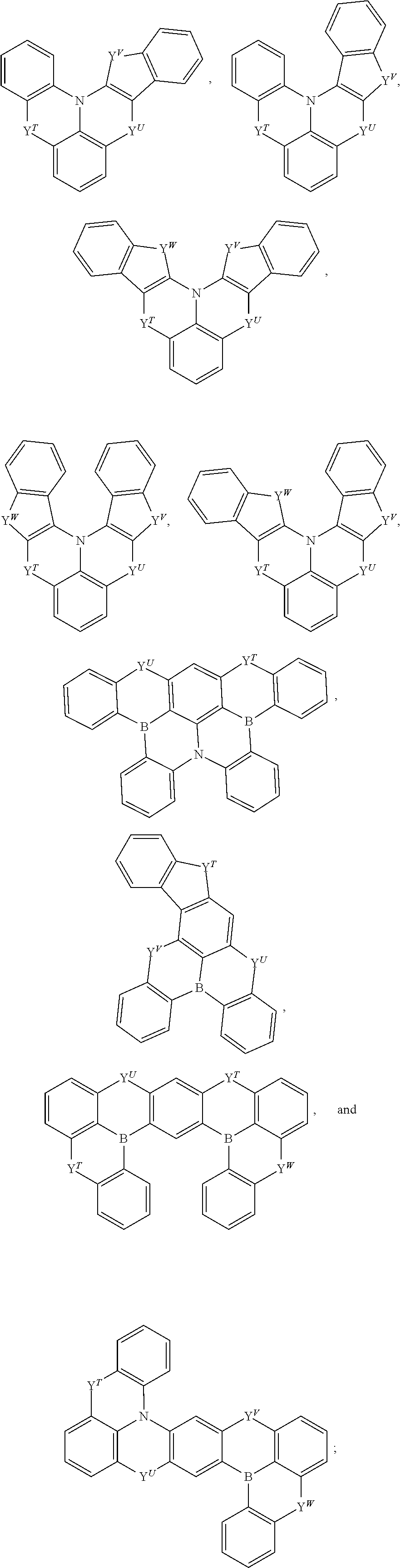

wherein each of Y^{T}, Y^{U}, Y^{V} and Y^{W} is independently selected from the group consisting of BR, NR, PR, O, S, Se, C═O, S═O, SO₂, BRR′, CRR′, SiRR′, and GeRR′;
wherein each R^{T} can be the same or different and each R^{T} is independently a donor, an acceptor group, an organic linker bonded to a donor, an organic linker bonded to an acceptor group, or a terminal group selected from the group consisting of alkyl, cycloalkyl, heteroalkyl, heterocycloalkyl, arylalkyl, aryl, heteroaryl, and combinations thereof; and
R, and R′ are each independently a hydrogen or a substituent selected from the group consisting of deuterium, halogen, alkyl, cycloalkyl, heteroalkyl, heterocycloalkyl, boryl, arylalkyl, alkoxy, aryloxy, amino, silyl, germyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof.

In some embodiments, any carbon ring atoms up to maximum of a total number of three, together with their substituents, in each phenyl ring of any of above structures can be replaced with N.

In some embodiments, the TADF material comprises at least one of the chemical moieties selected from the group consisting of nitrile, isonitrile, borane, fluoride, pyridine, pyrimidine, pyrazine, triazine, aza-carbazole, aza-dibenzothiophene, aza-dibenzofuran, aza-dibenzoselenophene, aza-triphenylene, imidazole, pyrazole, oxazole, thiazole, isoxazole, isothiazole, triazole, thiadiazole, and oxadiazole.

In some embodiments, the compound S1 and the compound A1 are present together in at least one emissive layer.

In some embodiments, the compound S1 and the compound A1 are in different emissive layers.

In some embodiments, acceptor A1 and sensitizer S1 are not present in any of the same emissive layers.

In some embodiments, the emissive layer comprising the compound S1 and the emissive layer comprising the compound A1 are in contact with each other.

In some embodiments, the emissive layer comprising the compound S1 and the emissive layer comprising the compound A1 are not in contact with each other.

In some embodiments, the emissive region comprises at least one emissive layer, and a concentration of compound A1 in each of the at least one emissive layer containing compound A1 is at least >2 vol-%.

In some embodiments, the emissive region comprises at least one emissive layer, and a concentration of compound A1 in each of the at least one emissive layer containing compound A1 is at least >3 vol-%.

In some embodiments, the emissive region comprises at least one emissive layer, and a concentration of compound A1 in each of the at least one emissive layer containing compound A1 is at least >4 vol-%.

In some embodiments, the emissive region comprises at least one emissive layer, and a concentration of the compound A1 in each of the at least one emissive layer containing the compound A1 is at least >5 vol-%.

In some embodiments, k_{FRET}>k_{NR,SI}, wherein k_{FRET} is the averaged Forster resonance energy transfer rate between sensitizer S1 and acceptor A1 in the emissive region and k_{NR,SI} is the non-radiative rate of a PMMA film doped with 1 wt-% of sensitizer S1.

In some embodiments, k_{FRET}, k_{NR,SI}, and k_{DEX} can be measured by transient PL and transient absorption measurements known in the art.

In some embodiments, k_{DEX}>k_{NR,SI}, wherein k_{DEX} is the averaged Dexter energy transfer rate between sensitizer S1 and acceptor A1.

In some embodiments, acceptor A1 produces at least 50% of the emission from the emissive region.

In some embodiments, this is determined by measuring the emission spectrum of each emitter separately, then expressing the emissive region spectrum as a linear sum of the individual emission spectra. Thus, the percentage of the emission produced by compound A1 (or another compound in the emissive region) can be determined by measuring the emission spectrum of compound A1 (or another compound) in the emissive region separately, and then the percentage of the emission produced by compound A1 (or another compound in the emissive region) can be expressed as a linear sum of the individual emission spectra in the emissive region.

In some embodiments, compound A1 produces at least 70% of the emission from the emissive region.

In some embodiments, compound A1 produces at least 90% of the emission from the emissive region.

In some embodiments, a full width at half maximum (FWHM) of the emissive region is at least 5 nm less than the FWHM of the compound S1 alone.

In some embodiments, a FWHM of the emissive region is at least 10 nm less than the FWHM of the compound S1.

In some embodiments, a FWHM of the emissive region is at least 15 nm less than the FWHM of the compound S1.

In some embodiments, a FWHM of the emissive region is at least 20 nm less than the FWHM of the compound S1.

In some embodiments, an M/T ratio of the emissive region is at least 0.02 larger than the MIT ratio of the sensitizer S1. The MIT ratio is a descriptor for the “narrowness” of the peak. M is the area of main peak, which is defined as the integration of the area of max peak wavelength (λₘₐₓ)+15 nm. T is total area of the spectrum, which is defined as the integration of entire spectrum. High M/T means a dopant has a narrow lineshape.

In some embodiments, an M/T ratio of the emissive region is at least 0.035 larger than the MIT ratio of the compound S1.

In some embodiments, an M/T ratio of the emissive region is at least 0.05 larger than the MIT ratio of the compound S1.

In some embodiments, the compound A1 is selected from the group consisting of the structures in LIST 1a, LIST 1c, or LIST 1d.

In some embodiments, the compound A1 is selected from the group consisting of the structures in LIST 2.

In some embodiments, the compound A1 is selected from the group consisting of the structures in LIST 3.

In some embodiments, the host may be an e-host or an h-host. In some embodiments, the host may comprise an electron donor material containing a component having characteristic of hole transporting and containing at least one of carbazolyl, arylamino, silyl, fluorenyl, spirofluorenyl, dibenzothienyl, and dibenzofurylaryl, phosphonooxy, and TP moiety. In some embodiments, the host may comprise an electron acceptor material containing a component having characteristic of electron transporting and containing at least one of boryl, pyridyl, pyrimidinyl, triazinyl, imidazolyl, phenanthroline, sulfone, heptazinyl, oxadiazolyl, cyano, and diphenyl moiety.

In some embodiments, the emissive region comprises a single emissive layer.

In some embodiments, the emissive region comprises two or more emissive layers.

In some embodiments, each of the two or more emissive layers are stacked directly on each other.

In some embodiments, each of the two or more emissive layers is separated by a charge generation layer.

In some embodiments, the emissive region comprises at least one host material.

In some embodiments, the emissive region further includes a third compound.

In some embodiments, the third compound is a host.

In some embodiments, the third compound is an H-host.

In some embodiments, the emissive region further includes a fourth compound.

In some embodiments, the fourth compound is a host.

In some embodiments, the fourth compound is an E-host.

In some embodiments, the first host, the second host, the third compound, or the fourth compound comprises at least one chemical group selected from the group consisting of triphenylene, carbazole, indolocarbazole, dibenzothiophene, dibenzofuran, dibenzoselenophene, 5λ²-benzo[d]benzo[4,5]imidazo[3,2-a]imidazole, 5,9-dioxa-13b-boranaphtho[3,2,1-de]anthracene, azaborinine, oxaborinine, dihydroacridine, xanthene, dihydrobenzoazasiline, dibenzooxasiline, phenoxazine, phenoxathiine, phenothiazine, dihydrophenazine, fluorene, naphthalene, anthracene, phenanthrene, phenanthroline, benzoquinoline, quinoline, isoquinoline, quinazoline, pyrimidine, pyrazine, pyridine, triazine, boryl, silyl, aza-triphenylene, aza-carbazole, aza-indolocarbazole, aza-dibenzothiophene, aza-dibenzofuran, aza-dibenzoselenophene, aza-5λ²-benzo[d]benzo[4,5]imidazo[3,2-a]imidazole, and aza-(5,9-dioxa-13b-boranaphtho[3,2,1-de]anthracene).

In some embodiments, the first host, the second host, the third compound, or the fourth compound is selected from the group consisting of the structures of the following LIST 13 defined herein:

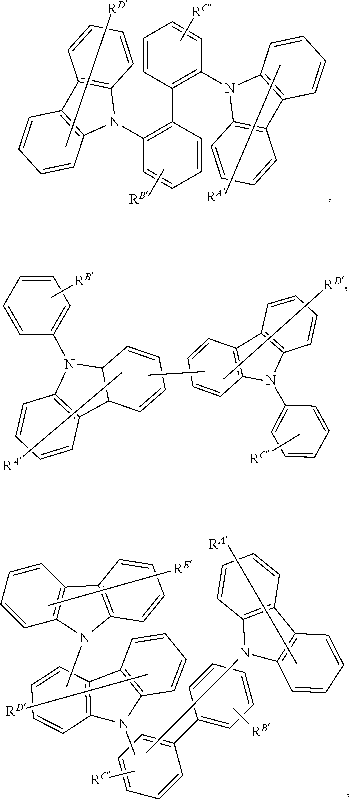

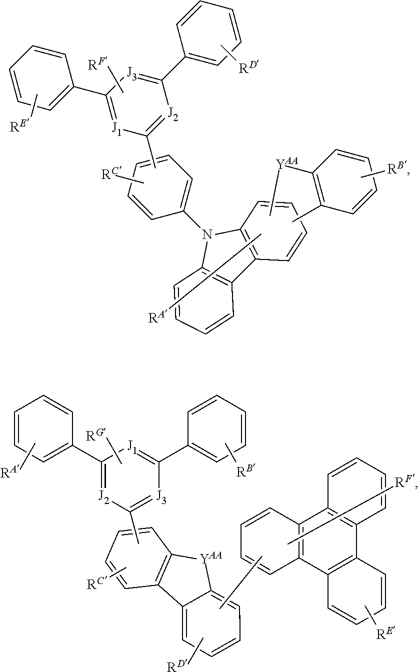

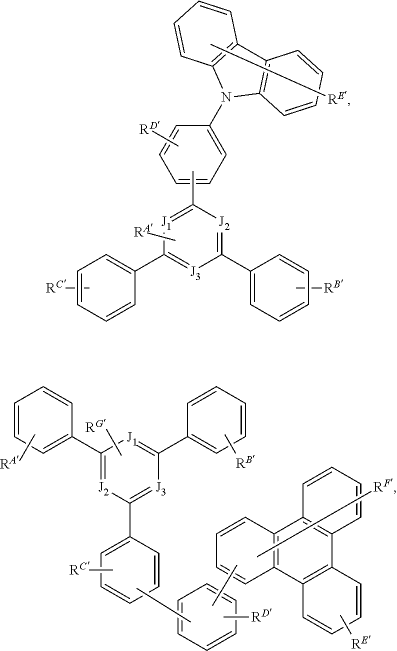

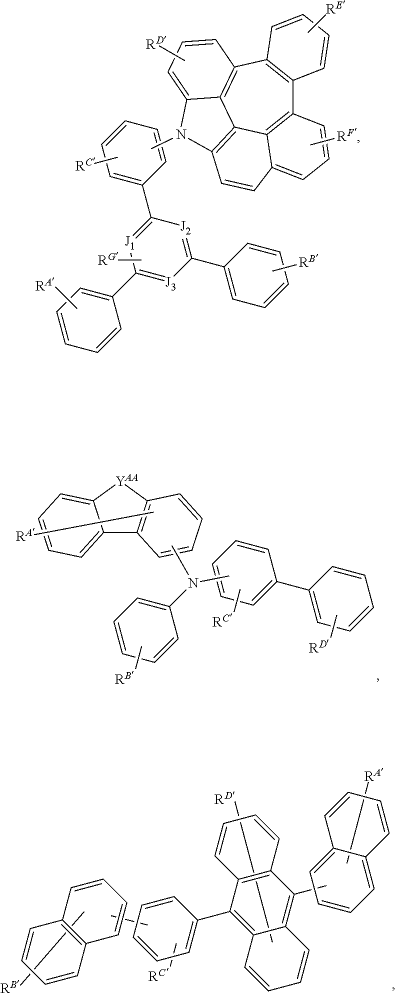

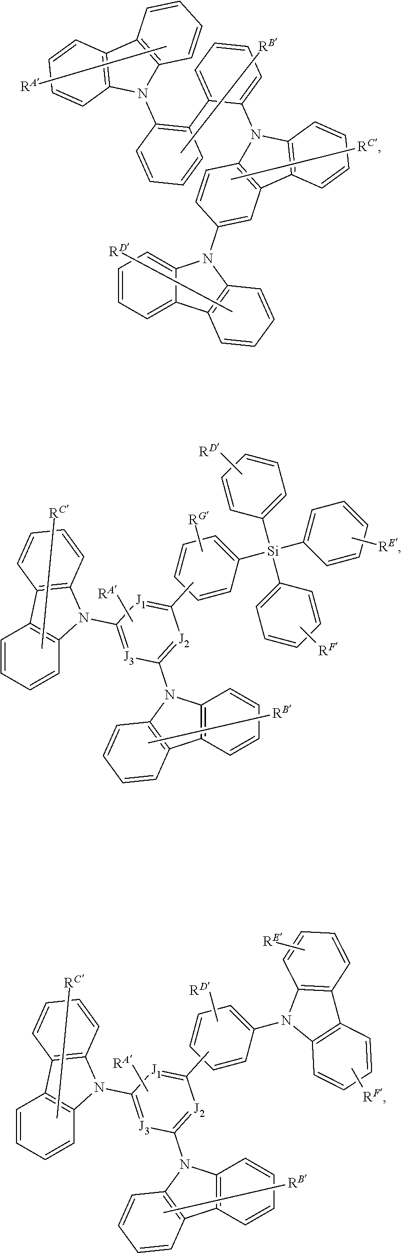

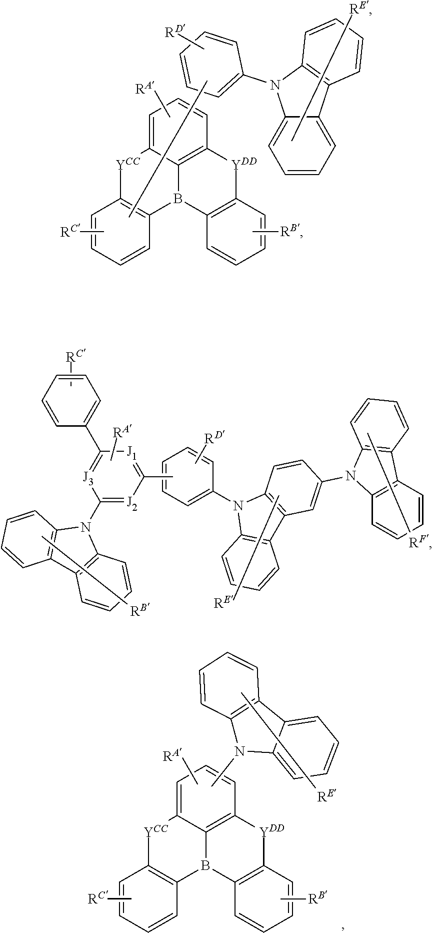

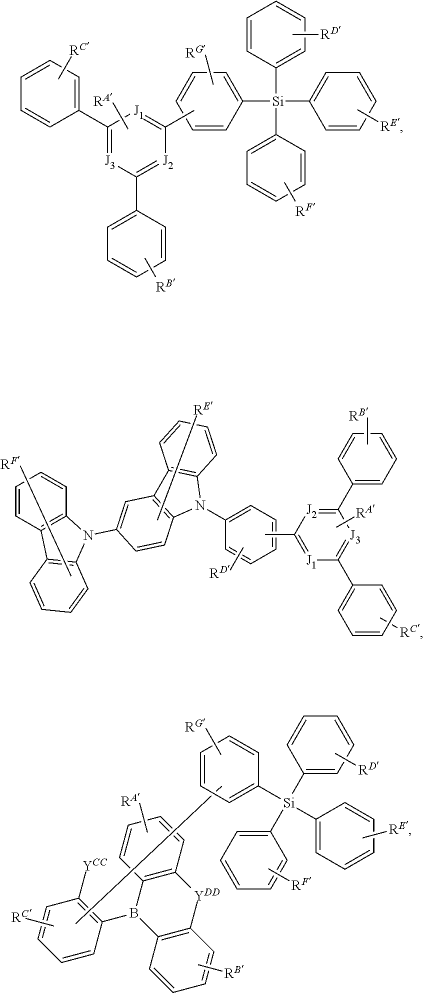

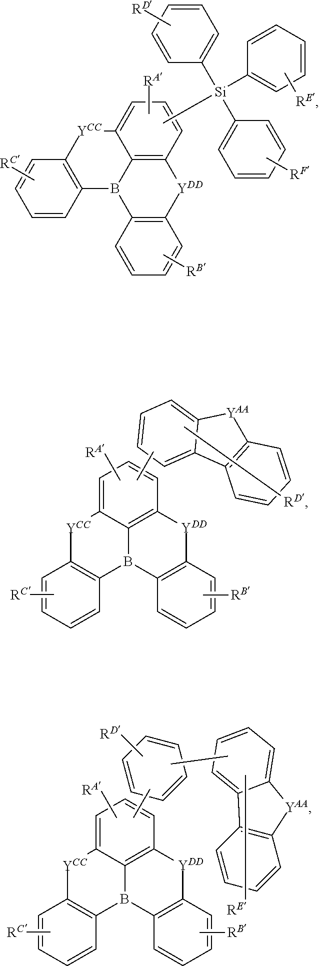

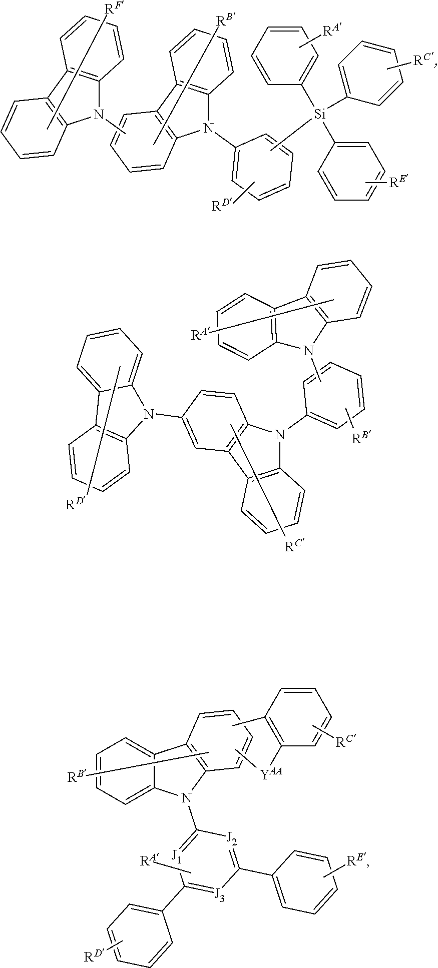

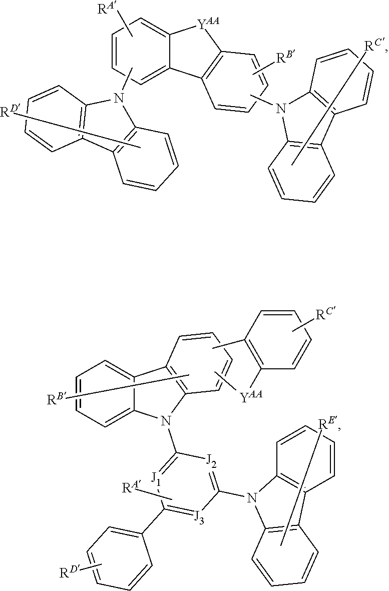

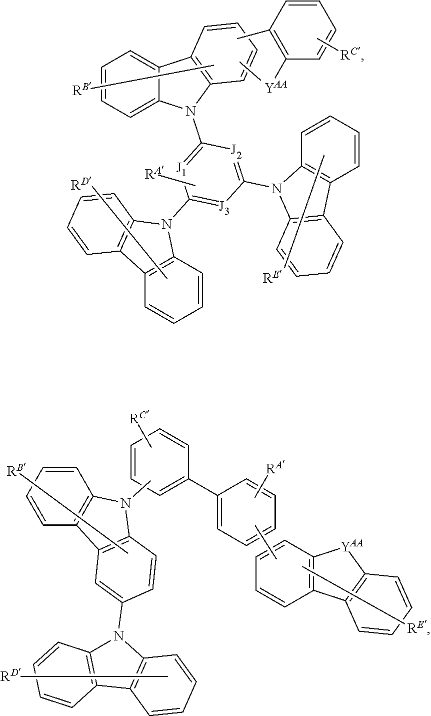

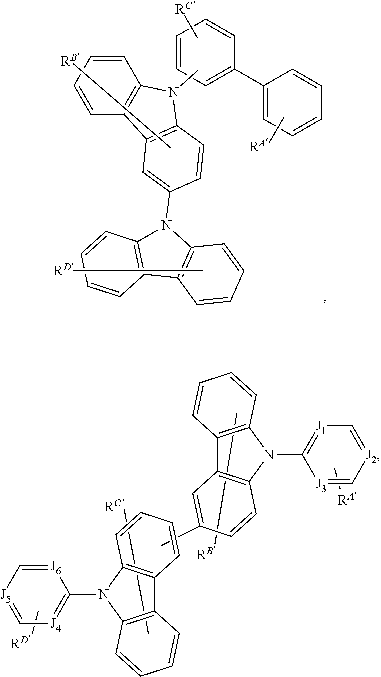

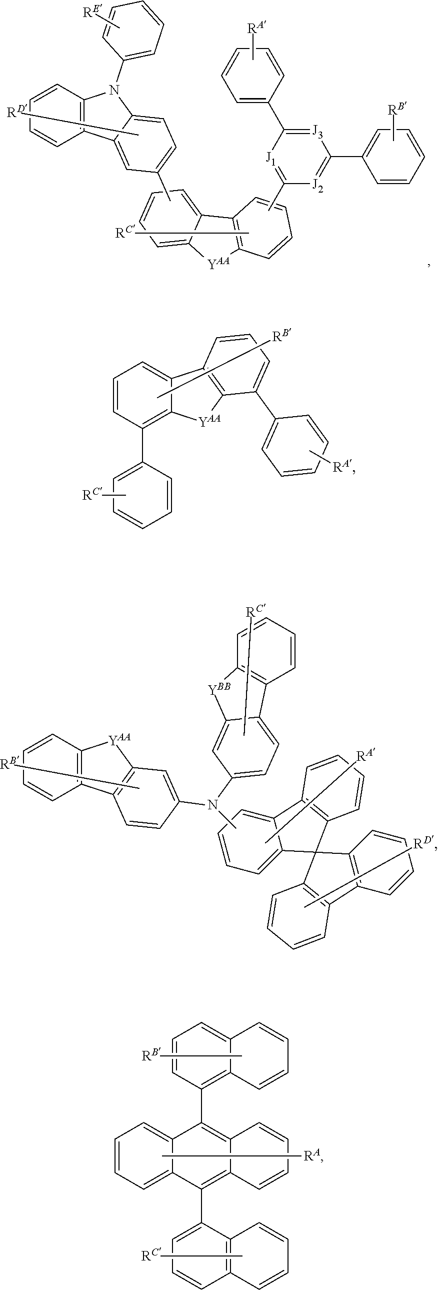

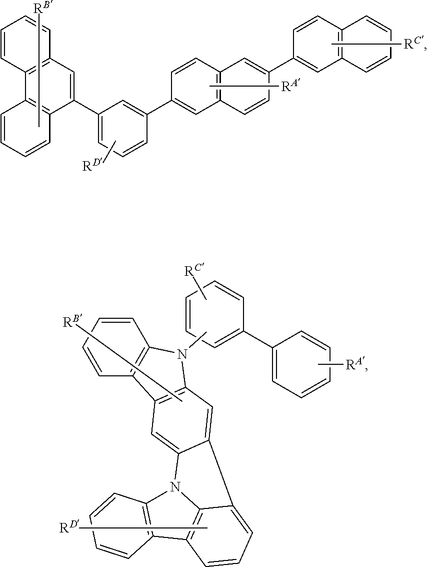

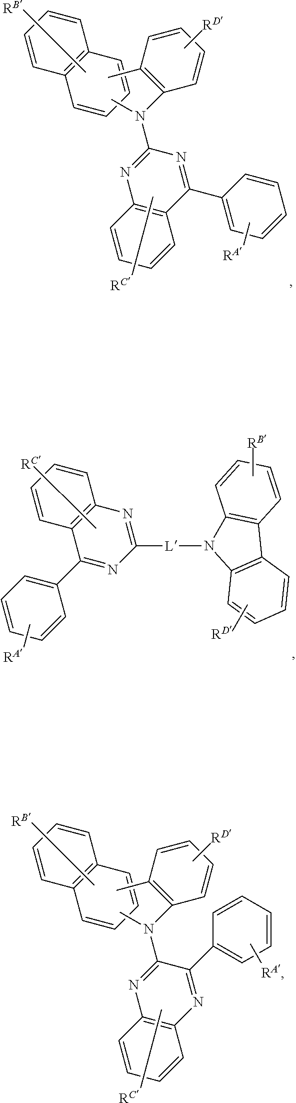

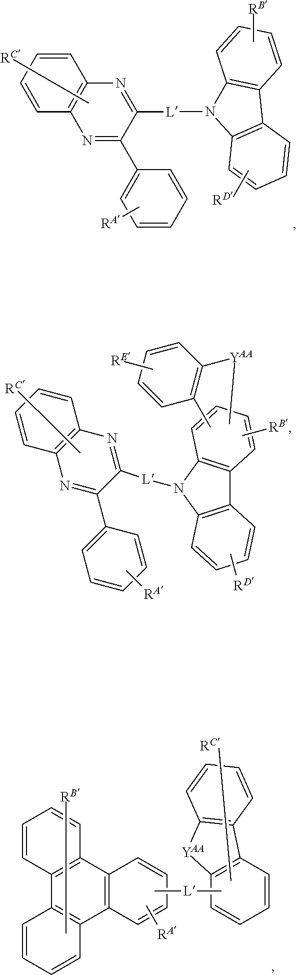

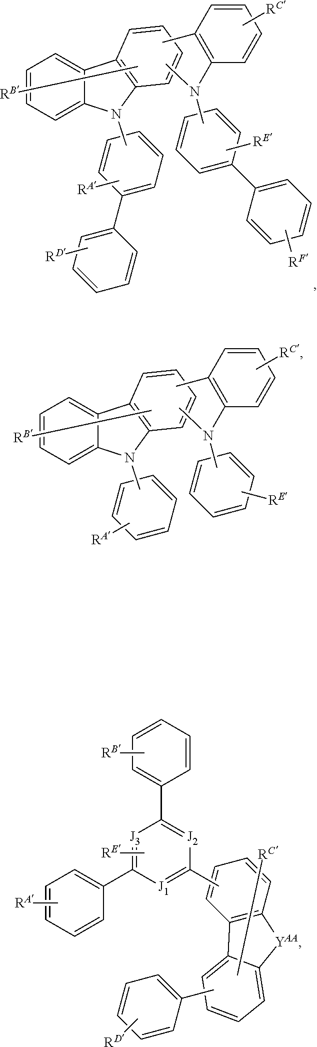

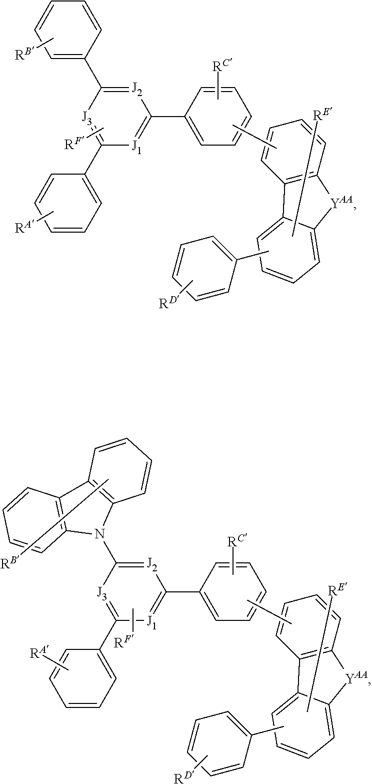

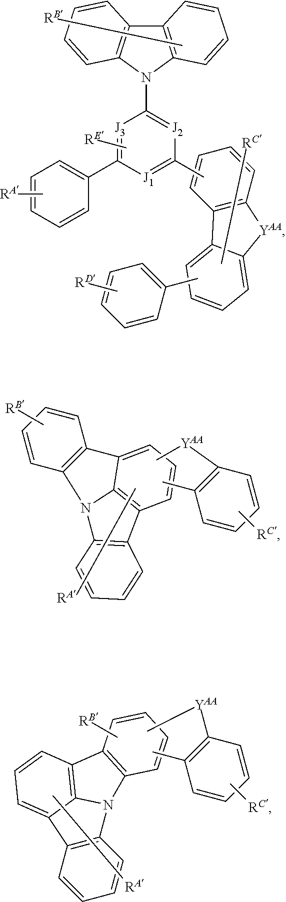

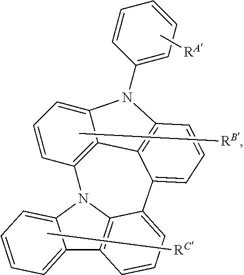

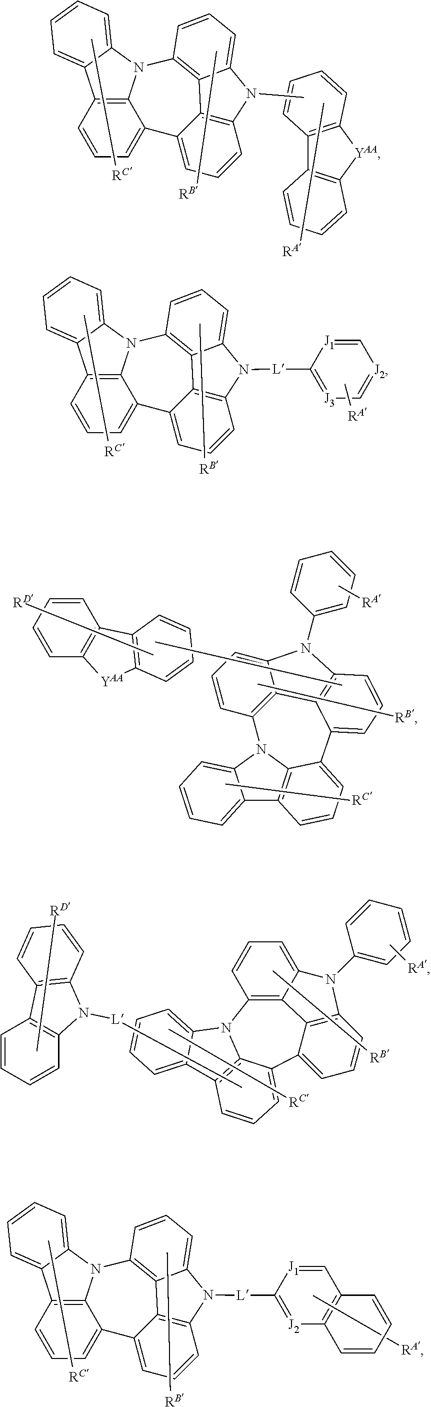

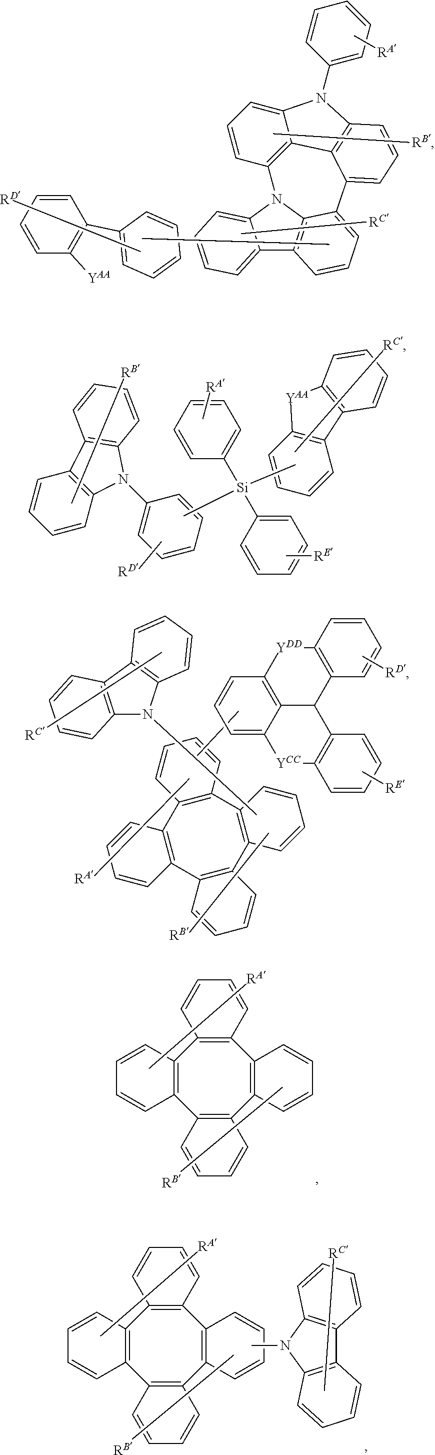

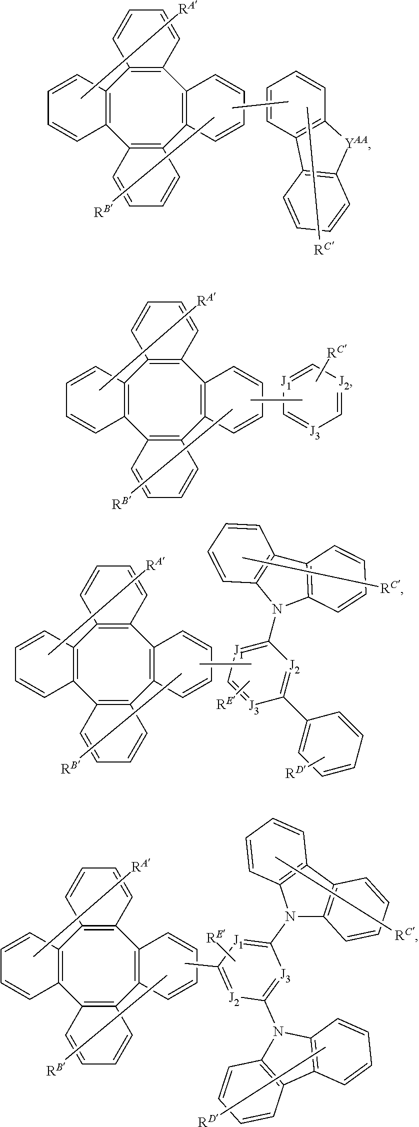

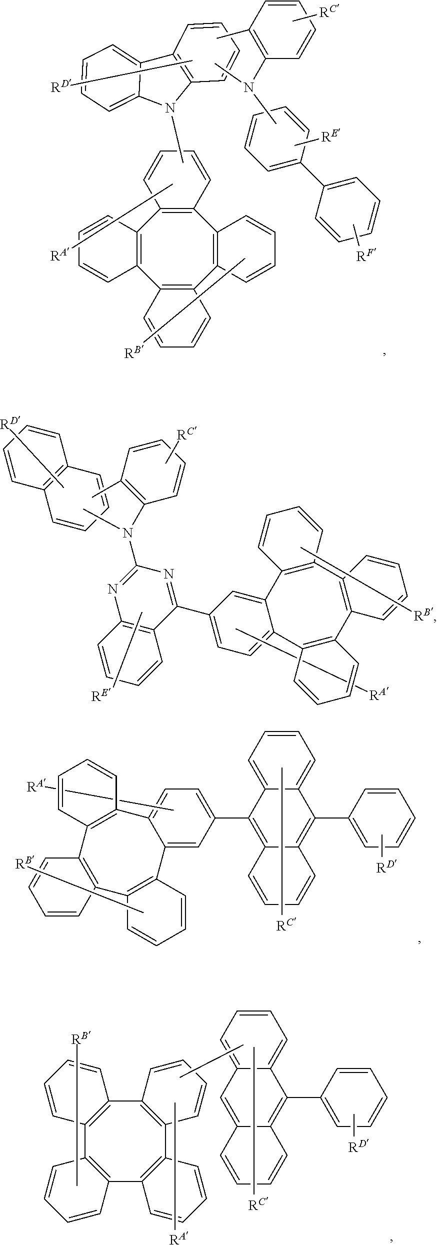

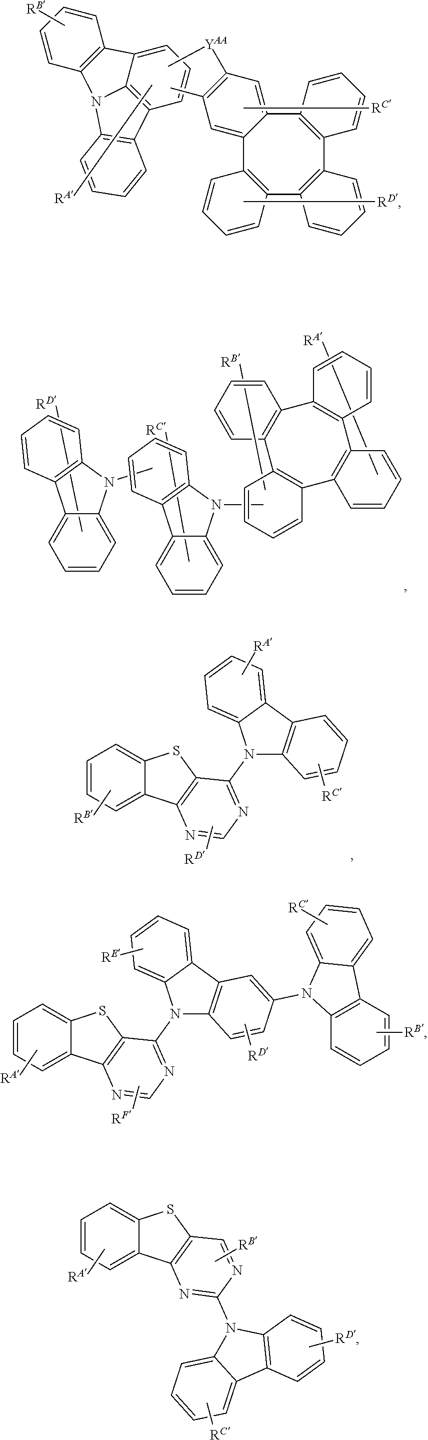

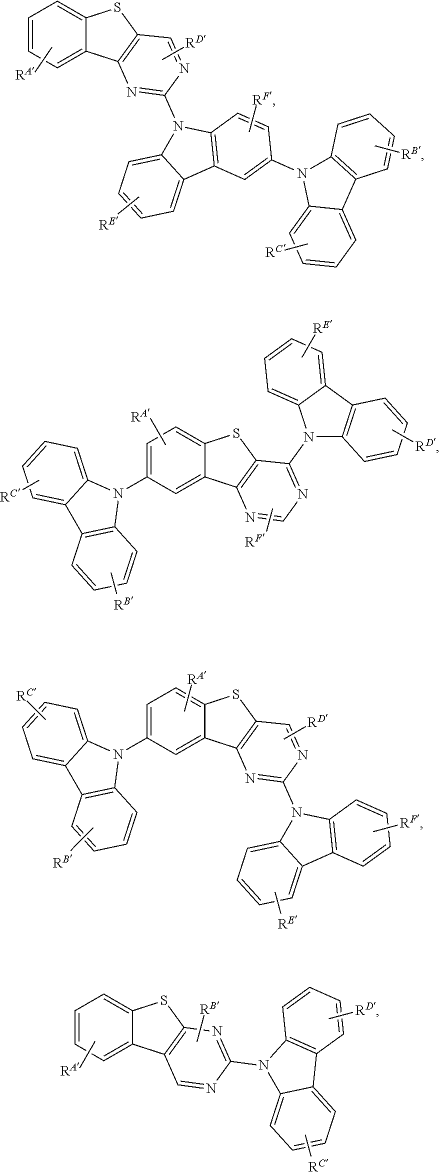

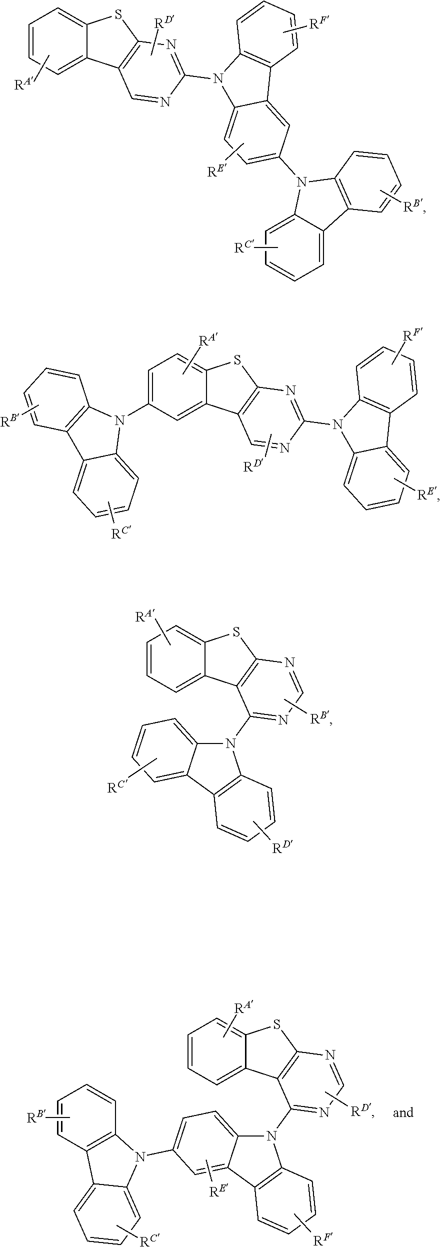

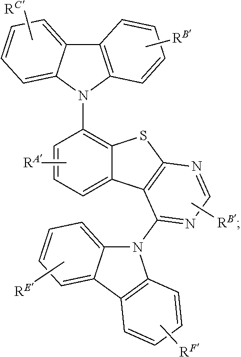

wherein:
each of J₁ to J₆ is independently C or N;
L′ is a direct bond or an organic linker;
each Y^{AA}, Y^{BB}, Y^{CC}, and Y^{DD} is independently selected from the group consisting of absent a bond, direct bond, O, S, Se, CRR′, SiRR′, GeRR′, NR, BR, BRR′;
each of R^{A′}, R^{B′}, R^{C′}, R^{D′}, R^{E′}, R^{F′}, and R^{G′} independently represents mono, up to the maximum substitutions, or no substitutions;
each R, R′, R^{A′}, R^{B′}, R^{C′}, R^{D′}, R^{E′}, R^{F′}, and R^{G′} is independently a hydrogen or a substituent selected from the group consisting of the General Substituents as defined herein; any two substituents can be joined or fused to form a ring; and
where possible, each unsubstituted aromatic carbon atom is optionally replaced with one or more N to form an aza-substituted ring.

In some embodiments, L′ is an organic linker selected from the group consisting of BR, BRR′, NR, PR, P(O)R, O, S, Se, C═O, C═S, C═Se, C═NR, C═CRR′, S═O, SO₂, CR, CRR′, SiRR′, GeRR′, alkylene, cycloalkyl, aryl, cycloalkylene, arylene, heteroarylene, and combinations thereof.

In some embodiments, the OLED comprises at least one layer from the group consisting of a hole injection layer, a hole transport layer, an electron blocking layer, an emission material layer, a hole blocking layer, an electron transport layer, electron injection layer and a capping layer.

In some embodiments, an OLED of the present disclosure comprises an emissive region disposed between the anode and the cathode; wherein the emissive region comprises a sensitizer compound and an acceptor compound; wherein the sensitizer transfers energy to the acceptor compound that is an emitter. In some embodiments, the sensitizer compound is capable of emitting light from a triplet excited state to a ground singlet state in an OLED at room temperature. In some embodiments, the sensitizer compound is capable of functioning as a phosphorescent emitter, a TADF emitter, or a doublet emitter in an OLED at room temperature, they are also referred as a phosphorescent material, a TADF material, or a doublet material. In some embodiments, the acceptor compound is selected from the group consisting of: a delayed-fluorescent compound functioning as a TADF emitter in the OLED at room temperature, i.e., a delayed-fluorescent material, a fluorescent compound functioning as a fluorescent emitter in the OLED at room temperature, i.e., a fluorescent material. In some embodiments, the fluorescent emitter can be a singlet or doublet emitters. In some of such embodiments, the singlet emitter can also include a TADF emitter, furthermore, a multi-resonant MR-TADF emitter. Description of the delayed fluorescence as used herein can be found in U.S. application publication US20200373510A1, at paragraphs 0083-0084, the entire contents of which are incorporated herein by reference.

In some embodiments of the OLED, the sensitizer and acceptor compounds are in separate layers within the emissive region.

In some embodiments, the sensitizer and the acceptor compounds are present as a mixture in one or more layers in the emissive region. It should be understood that the mixture in a given layer can be a homogeneous mixture or the compounds in the mixture can be in graded concentrations through the thickness of the given layer. The concentration grading can be linear, non-linear, sinusoidal, etc. When there are more than one layer in the emissive region having a mixture of the sensitizer and the acceptor compounds, the type of mixture (i.e., homogeneous or graded concentration) and the concentration levels of the compounds in the mixture in each of the more than one layer can be the same or different. In addition to the sensitizer and the acceptor compounds, there can be one or more other functional compounds such as, but not limit to, hosts also mixed into the mixture.

In some embodiments, the acceptor compound can be in two or more layers with the same or different concentration. In some embodiments, when two or more layers contain the acceptor compound, the concentrations of the acceptor compound in at least two of the two or more layers are different. In some embodiments, the concentration of sensitizer compound in the layer containing the sensitizer compound is in the range of 1 to 50%, 10 to 20%, or 12-15% by weight. In some embodiments, the concentration of the acceptor compound in the layer containing the acceptor compound is in the range of 0.1 to 10%, 0.5 to 5%, or 1 to 3% by weight.

In some embodiments, the emissive region contains N layers where N>2. In some embodiments, the sensitizer compound is present in each of the N layers, and the acceptor compound is contained in fewer than or equal to N−1 layers. In some embodiments, the sensitizer compound is present in each of the N layers, and the acceptor compound is contained in fewer than or equal to N/2 layers. In some embodiments, the acceptor compound is present in each of the N layers, and the sensitizer compound is contained in fewer than or equal to N−1 layers. In some embodiments, the acceptor compound is present in each of the N layers, and the sensitizer compound is contained in fewer than or equal to N/2 layers.

In some embodiments, the OLED emits a luminescent emission comprising an emission component from the S₁ energy (the first singlet energy) of the acceptor compound when a voltage is applied across the OLED. In some embodiments, at least 65%, 75%, 85%, or 95% of the emission from the OLED is produced from the acceptor compound with a luminance of at least 10 cd/m². In some embodiments, S₁ energy of the acceptor compound is lower than that of the sensitizer compound.

In some embodiments, a T₁ energy (the first triplet energy) of the host compound is greater than or equal to the T₁ energies of the sensitizer compound and the acceptor compound, and the T₁ energy of the sensitizer compound is greater than or equal to the S₁ energy (the first singlet energy) of the acceptor compound. In some embodiments, S₁-T₁ energy gap of the sensitizer compound, and/or acceptor compound, and/or first host compound, and/or second host compound is less than 400, 300, 250, 200, 150, 100, or 50 meV. In some embodiments, the absolute energy difference between the HOMO of the sensitizer compound and the HOMO of the acceptor compound is less than 0.6, 0.5, 0.4, 0.3, or 0.2 eV. In some embodiments, the absolute energy difference between the LUMO of the sensitizer compound and the LUMO of the acceptor compound is less than 0.6, 0.5, 0.4, 0.3, or 0.2 eV.

Generally, T1 energy, HOMO and LUMO can be obtained by experimental measurements, and those measurements (numbers) are to be used for the related purposes, intentions, and/or embodiments unless specifically stipulated otherwise. More particularly, solution cyclic voltammetry and differential pulsed voltammetry can be performed using a CH Instruments model 6201B potentiostat using anhydrous dimethylformamide solvent and tetrabutylammonium hexafluorophosphate as the supporting electrolyte. Glassy carbon, and platinum and silver wires can be used as the working, counter and reference electrodes, respectively. Electrochemical potentials are referenced to an internal ferrocene-ferroconium redox couple (Fc/Fc+) by measuring the peak potential differences from differential pulsed voltammetry. The corresponding highest occupied molecular orbital (HOMO) and lowest unoccupied molecular orbital (LUMO) energies can then be determined by referencing the cationic and anionic redox potentials to ferrocene (4.8 eV vs. vacuum) according to literature ((a) Fink, R.; Heischkel, Y.; Thelakkat, M.; Schmidt, H.-W. Chem. Mater. 1998, 10, 3620-3625. (b) Pommerehne, J.; Vestweber, H.; Guss, W.; Mahrt, R. F.; Bassler, H.; Porsch, M.; Daub, J. Adv. Mater. 1995, 7, 551.

The T1 energy can be obtained from the emission spectrum of a frozen sample in 2-MeTHF at 77 K.

Emission spectra were collected on a Horiba Fluorolog-3 spectrofluorometer equipped with a Synapse Plus CCD detector. All samples were excited at 340 nm. Transient data was measured by time correlated single photon counting (TCSPC) in the Fluorolog-3 using a 335 nm NanoLED pulsed excitation source. PLQY values were measured using a Hamamatsu Quantaurus-QY Plus UV-NIR absolute PL quantum yield spectrometer with an excitation wavelength of 340 nm. Solutions of 1% emitter with PMMA in toluene were prepared, filtered, and dropcast onto Quartz substrates.

In some embodiments where the sensitizer compound provides unicolored sensitization (i.e., minimal loss in energy upon energy transfer to the acceptor compound), the acceptor compound has a Stokes shift of 30, 25, 20, 15, or 10 nm or less. An example would be a broad blue phosphor sensitizing a narrow blue emitting acceptor.

In some embodiments where the sensitizer compound provides a down conversion process (e.g., a blue emitter being used to sensitize a green emitter, or a green emitter being used to sensitize a red emitter), the acceptor compound has a Stokes shift of 30, 40, 60, 80, or 100 nm or more.

In some embodiments, the difference between λmax of the emission spectrum of compound S1 and λmax of the absorption spectrum of the compound A1 is 50, 40, 30, or 20 nm or less. In some embodiments, the difference between λmax of the emission spectrum of the highest energy emission peak of compound S1 and λmax of the absorption spectrum of the lowest energy absorption peak of the compound A1 is 50, 40, 30, or 20 nm or less. In some embodiments, the area of spectral overlap of the absorption spectrum of compound A1 normalized to the lowest energy absorption peak and the emission spectrum of the compound S1 normalized to the highest energy emission peak relative to the area of the emission spectrum of the compound S1 normalized to the highest energy emission peak is greater than 5%, 10%, 15%, 20%, 30%, 40%, 50%, or more.

One way to quantify the qualitative relationship between a sensitizer compound (a compound to be used as the sensitizer in the emissive region of the OLED of the present disclosure) and an acceptor compound (a compound to be used as the acceptor in the emissive region of the OLED of the present disclosure) is by determining a value Δλ=λₘₐₓ₁−λₘₐₓ₂, where λₘₐₓ₁ and λₘₐₓ₂ are defined as follows. λₘₐₓ₁ is the emission maximum of the sensitizer compound at room temperature when the sensitizer compound is used as the sole emitter in a first monochromic OLED (an OLED that emits only one color) that has a first host. λₘₐₓ₂ is the emission maximum of the acceptor compound at room temperature when the acceptor compound is used as the sole emitter in a second monochromic OLED that has the same first host.

In some embodiments of the OLED of the present disclosure where the sensitizer compound provides unicolored sensitization (i.e., minimal loss in energy upon energy transfer to the acceptor compound), Δλ (determined as described above) is equal to or less than the number selected from the group consisting of 15, 12, 10, 8, 6, 4, 2, 0, −2, −4, −6, −8, and −10 nm.

In some embodiments, a spectral overlap integral of the acceptor compound and the sensitizer compound is at least 10¹⁴ nm^{4*}L/cm*mol. In some embodiments, a spectral overlap integral of the acceptor compound and the sensitizer compound is at least 5×10¹⁴ nm^{4*}L/cm*mol. In some embodiments, a spectral overlap integral of the acceptor compound and the sensitizer compound is at least 10¹⁵ nm^{4*}L/cm*mol.

As used herein, “spectral overlap integral” is determined by multiplying the acceptor compound extinction spectrum by the sensitizer compound emission spectrum normalized with respect to the area under the curve. The higher the spectral overlap, the better the Forster Resonance Energy Transfer (FRET) efficiency. The rate of FRET is proportional to the spectral overlap integral. Therefore, a high spectral overlap can help improve the FRET efficiency and reduce the exciton lifetime in an OLED.

In some embodiments, the acceptor compound and the sensitizer compound are selected in order to increase the spectral overlap. Increasing the spectral overlap can be achieved in several ways, for example, increasing the oscillator strength of the acceptor compound, minimizing the distance between the sensitizer compound peak emission intensity and the acceptor compound absorption peak, and narrowing the line shape of the sensitizer compound emission or the acceptor compound absorption. In some embodiments, the oscillator strength of the acceptor compound is greater than or equal to 0.1.

In some embodiments where the emission of the acceptor is redshifted by the sensitization, the absolute value of A is equal to or greater than the number selected from the group consisting of 20, 30, 40, 60, 80, 100 nm.

In the embodiments, the sensitizer compound is a phosphorescent material, and the sensitizer compound can be a metal coordination complex having a metal-carbon bond, a metal-nitrogen bond, or a metal-oxygen bond. In some embodiments, the metal is selected from the group consisting of Ir, Rh, Re, Ru, Os, Pt, Pd, Zn, Zr, Au, Ag, and Cu. In some embodiments, the metal is Ir. In some embodiments, the metal is Pt. In some embodiments, the sensitizer compound has the formula of M(L¹)ₓ(L²)_{y}(L³)_{z};
wherein L¹, L², and L³ can be the same or different;
wherein x is 1, 2, or 3;
wherein y is 0, 1, or 2;
wherein z is 0, 1, or 2;
wherein x+y+z is the oxidation state of the metal M;
wherein L¹ is selected from the group consisting of the structures of LIGAND LIST:

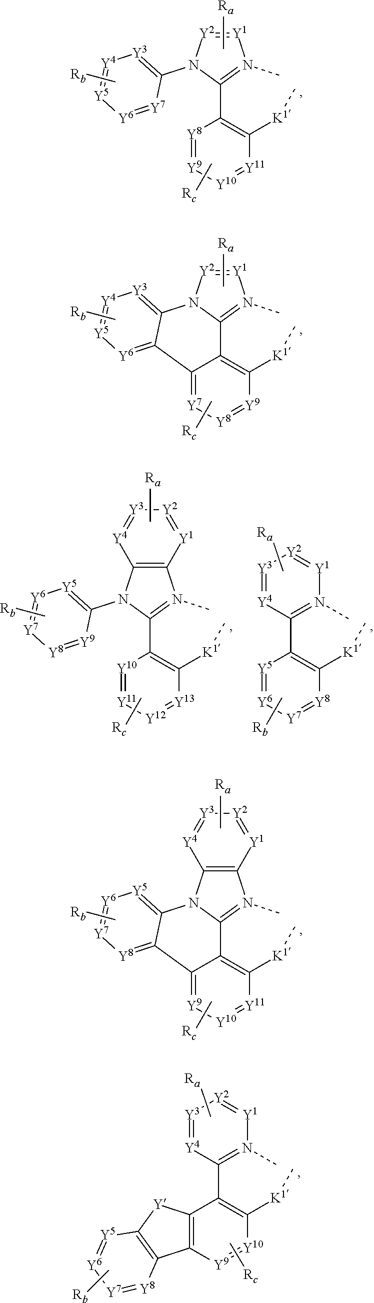

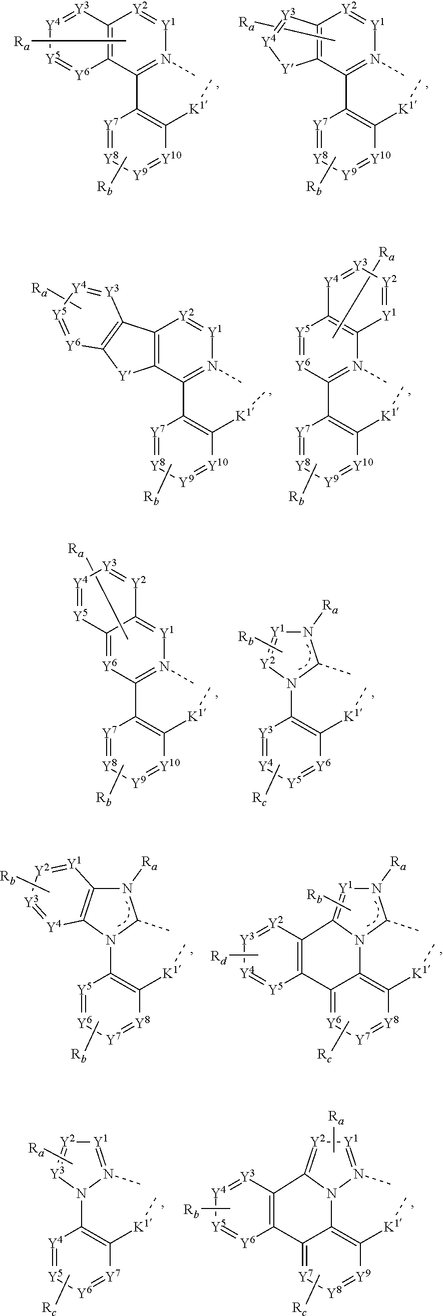

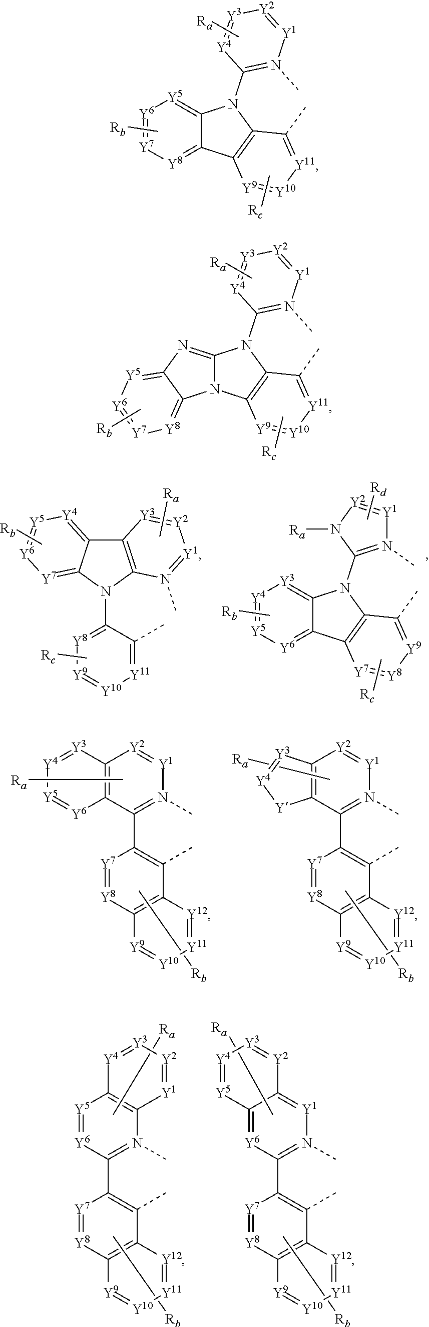

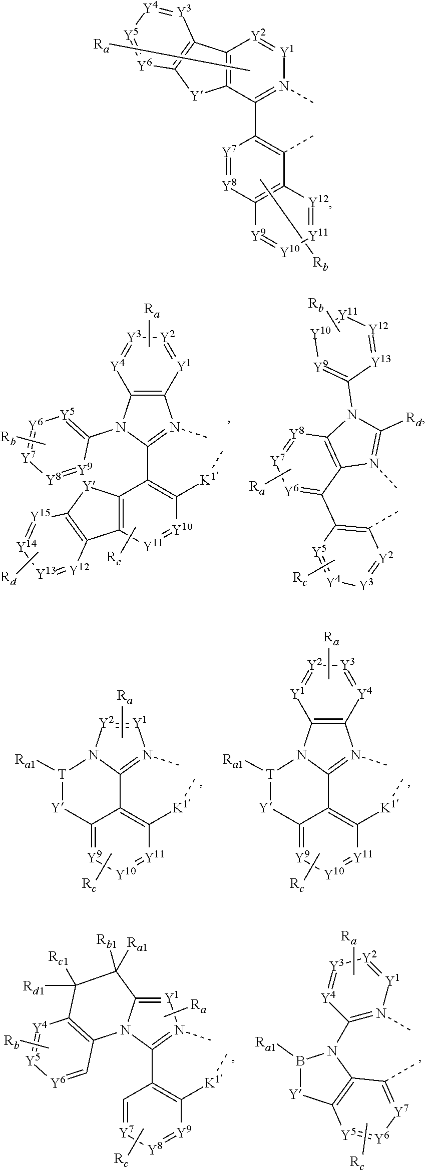

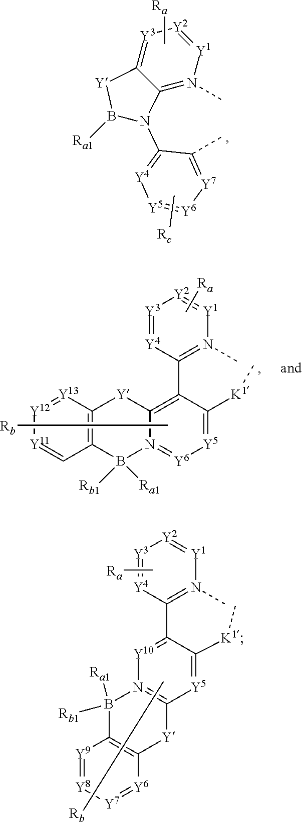

wherein L² and L³ are independently selected from the group consisting of

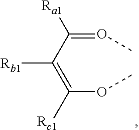

and the structures of LIGAND LIST; wherein:
   T is selected from the group consisting of B, A1, Ga, and In;
   K^{1′} is a direct bond or is selected from the group consisting of NRₑ, PRₑ, O, S, and Se;
   each Y¹ to Y¹³ are independently selected from the group consisting of carbon and nitrogen;
   Y′ is selected from the group consisting of BRₑ, NRₑ, PRₑ, O, S, Se, C═O, S═O, SO₂, CRₑR_{f}, SiRₑR_{f}, and GeRₑR_{f};
   Rₑ and R_{f} can be fused or joined to form a ring;
   each Rₐ, R_{b}, R_{c}, and R_{d} can independently represent from mono to the maximum possible number of substitutions, or no substitution;
   each Rₐ₁, R_{b1}, R_{c1}, R_{d1}, Rₐ, R_{b}, R_{c}, R_{d}, Rₑ, and R_{f} is independently a hydrogen or a substituent selected from the group consisting of the General Substituents as defined herein; and wherein any two of Rₐ₁, R_{b1}, R_{c1}, R_{d1}, Rₐ, R_{b}, R_{c}, and R_{d} can be fused or joined to form a ring or form a multidentate ligand.

In some embodiments, the metal in formula M(L¹)ₓ(L²)_{y}(L³)_{z} is selected from the group consisting of Cu, Ag, or Au.

In some embodiments of the OLED, the sensitizer compound has a formula selected from the group consisting of Ir(L_{A})₃, Ir(L_{A})(L_{B})₂, Ir(L_{A})₂(L_{B}), Ir(L_{A})₂(L_{C}), Ir(L_{A})(L_{B})(L_{C}), and Pt(L_{A})(L_{B});
wherein L_{A}, L_{B}, and L_{C} are different from each other in the Ir compounds;
wherein L_{A} and L_{B} can be the same or different in the Pt compounds; and
wherein L_{A} and L_{B} can be connected to form a tetradentate ligand in the Pt compounds.

In some embodiments of the OLED, the sensitizer compound is selected from the group consisting of the compounds in the following SENSITIZER LIST:

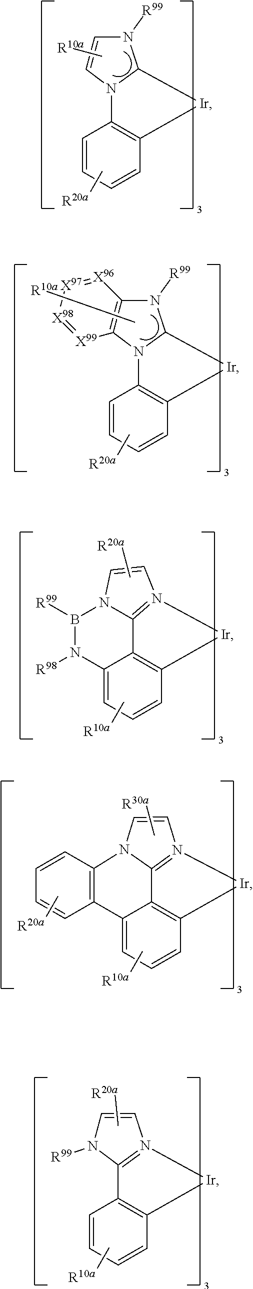

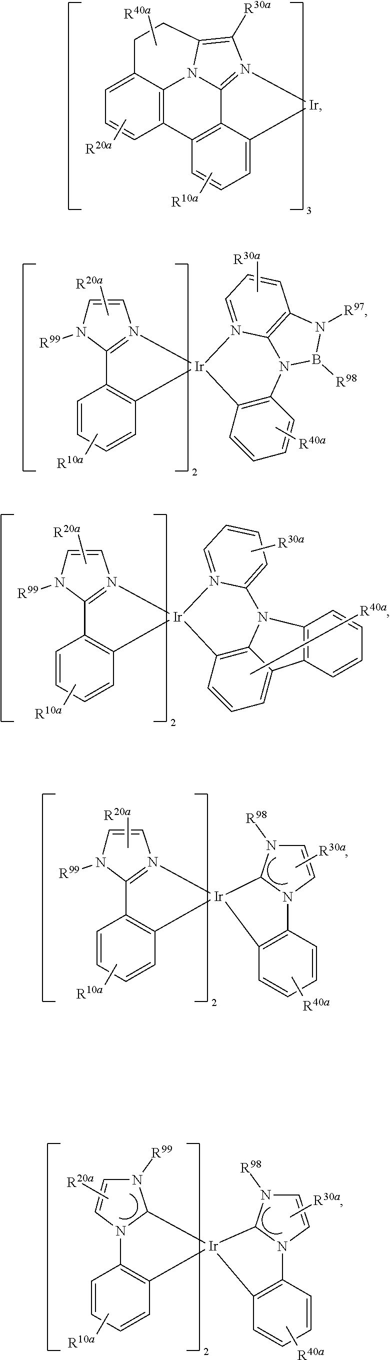

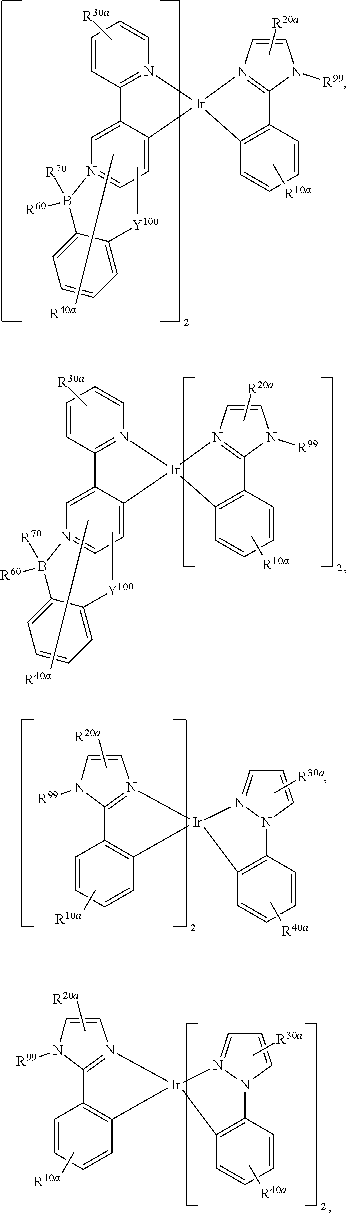

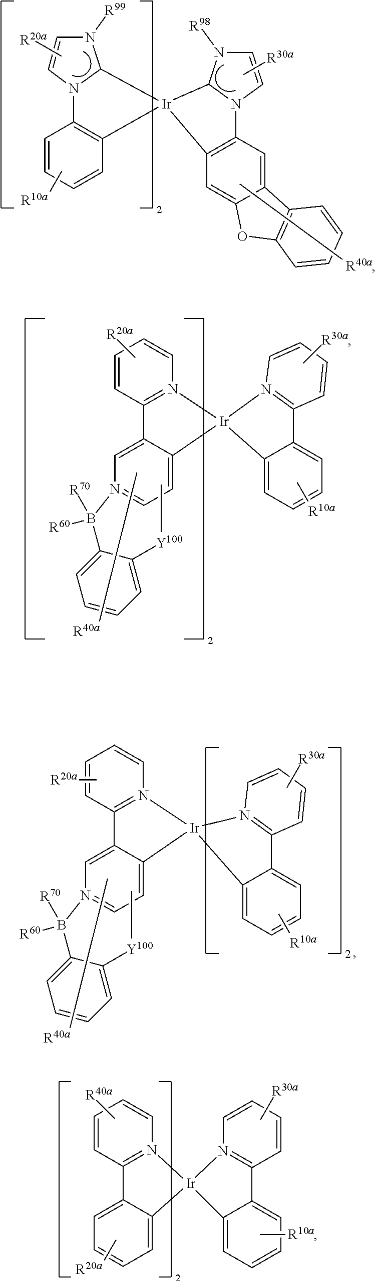

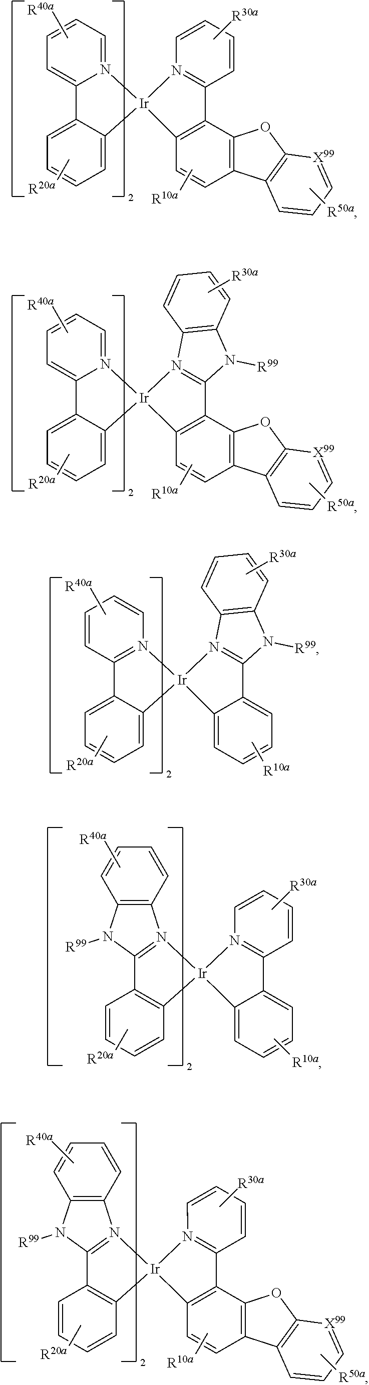

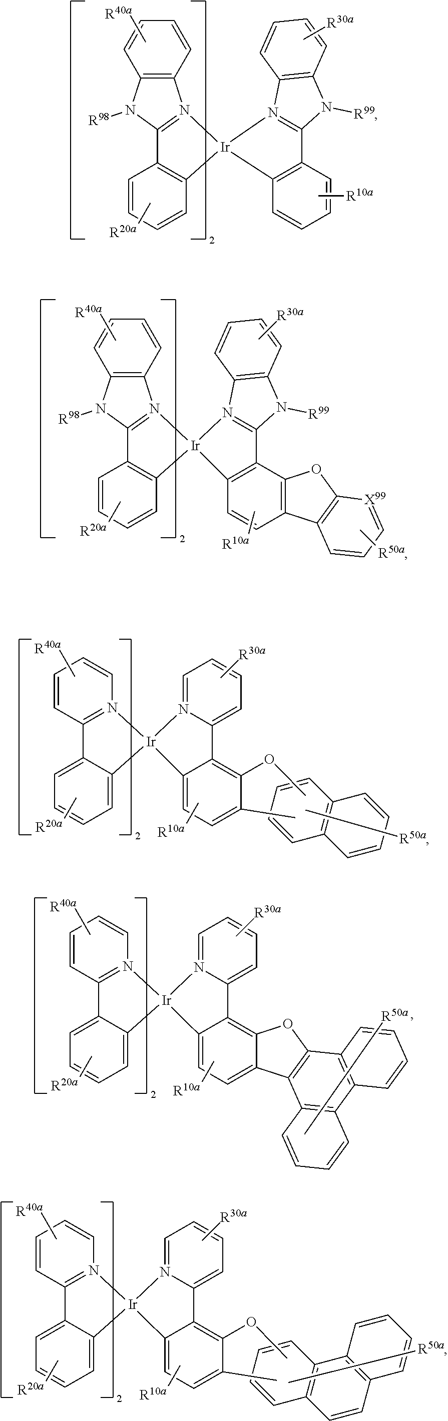

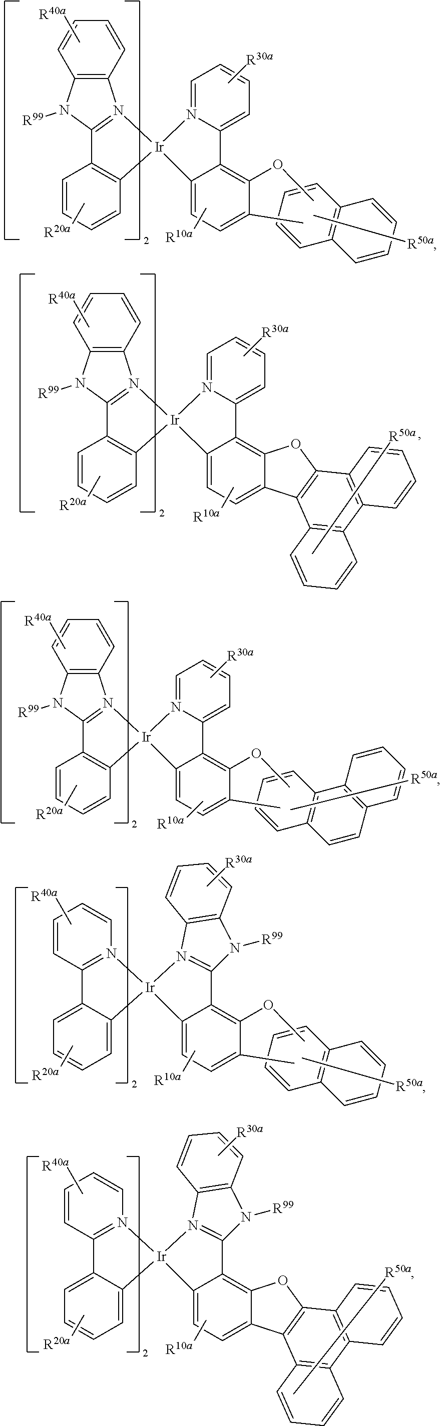

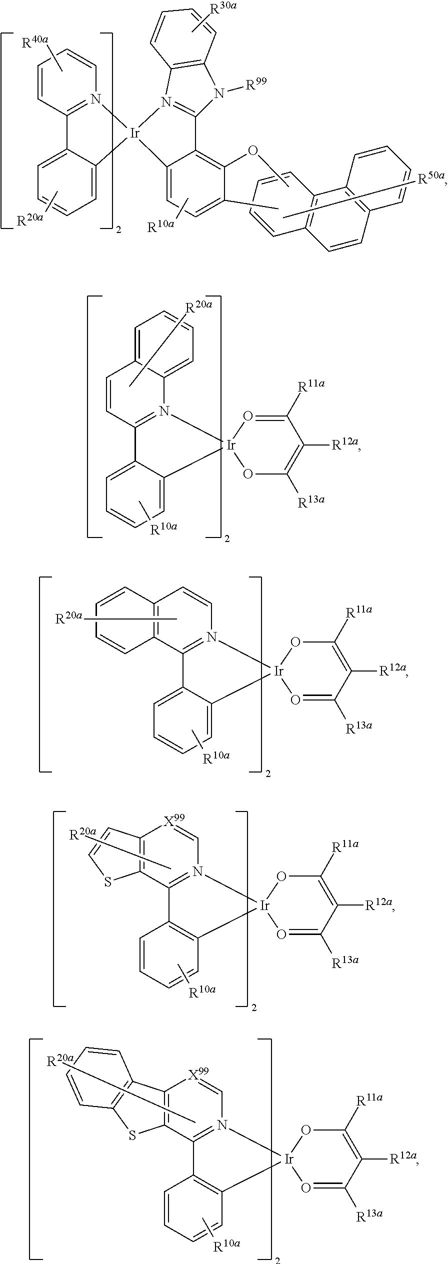

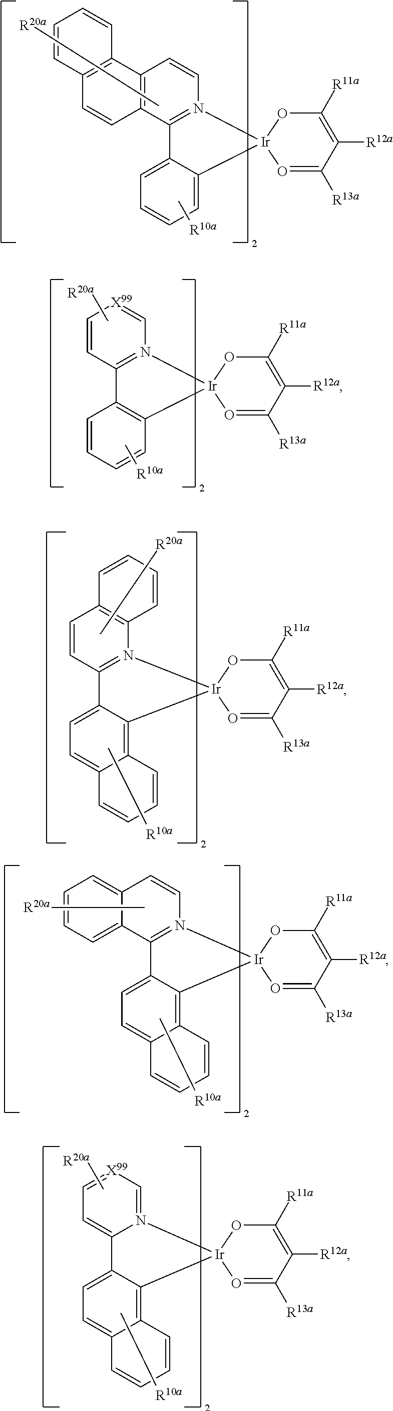

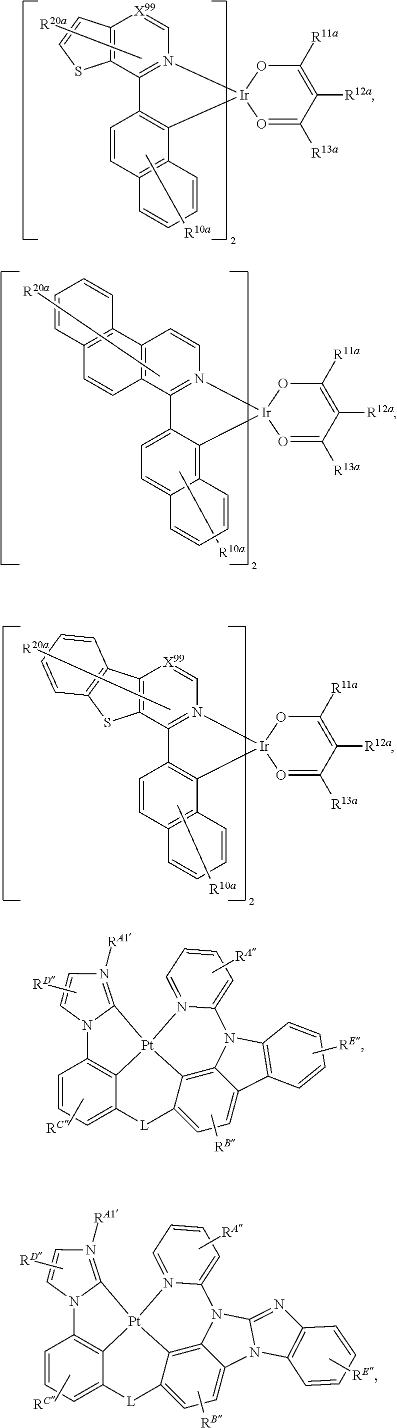

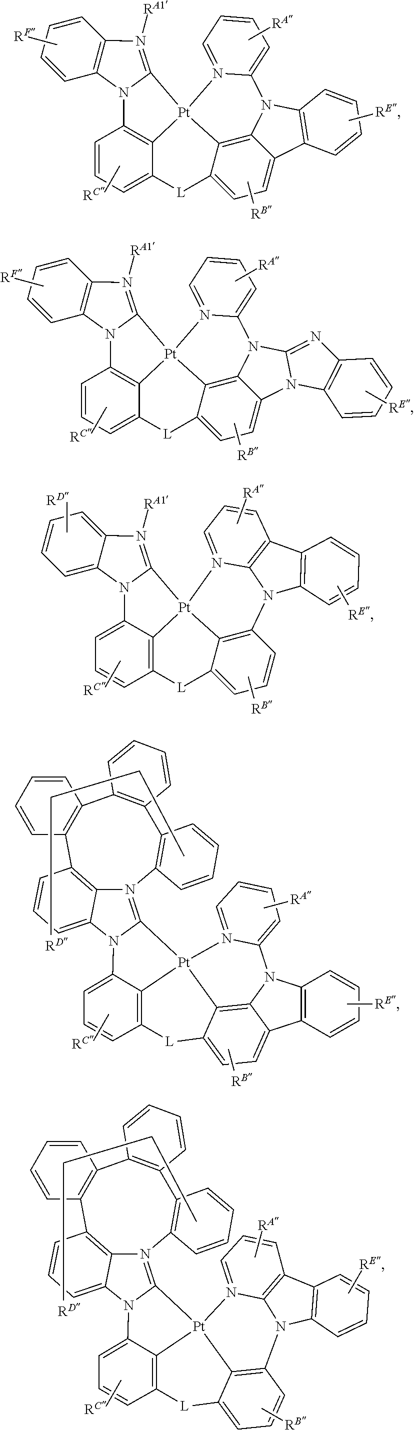

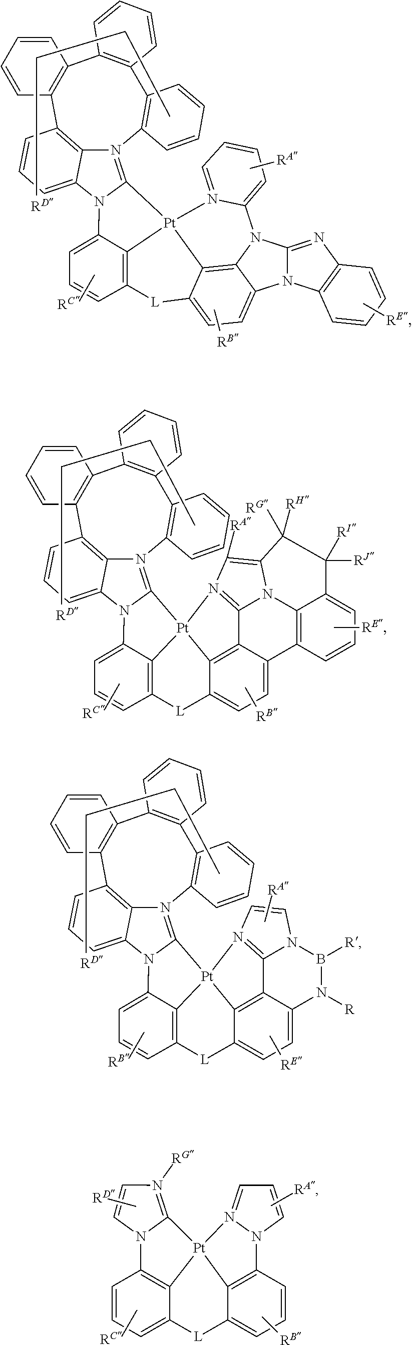

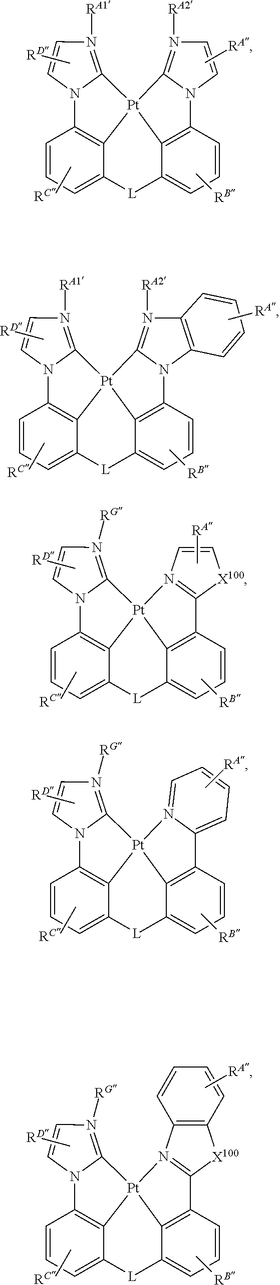

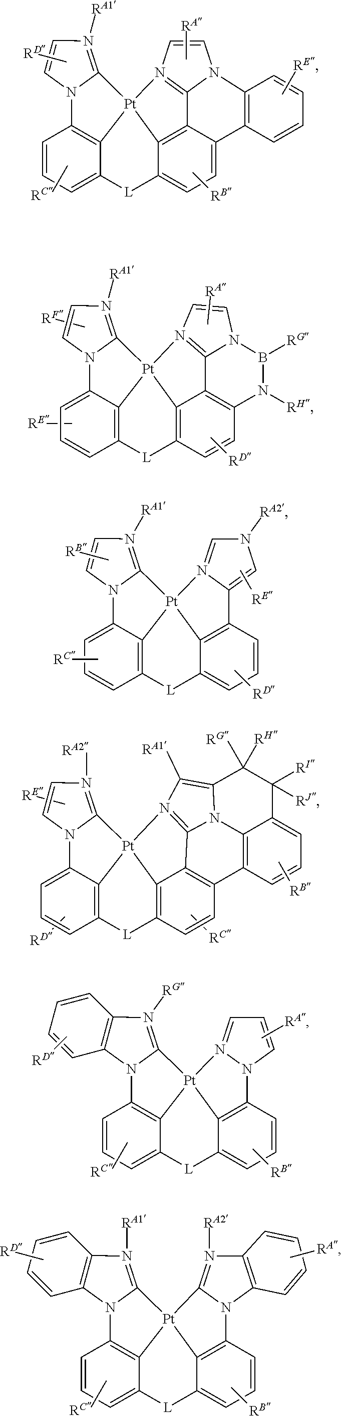

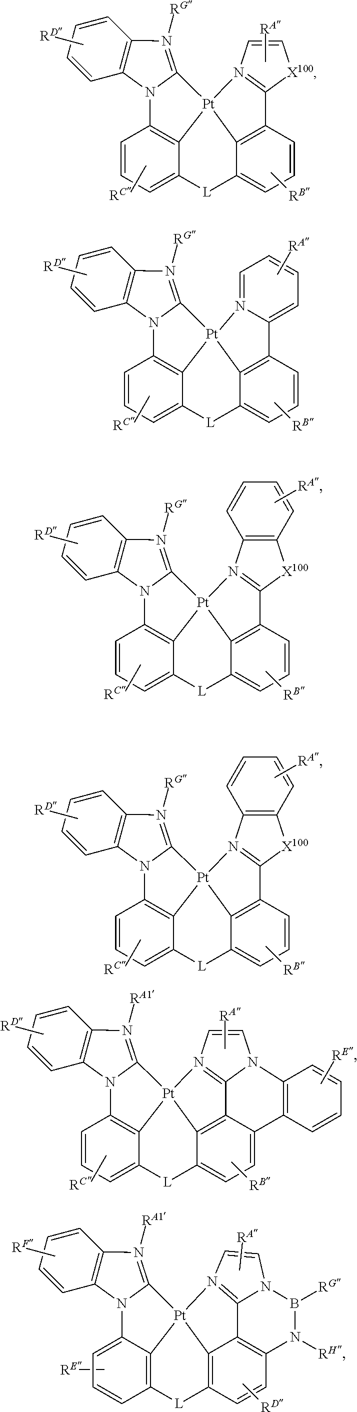

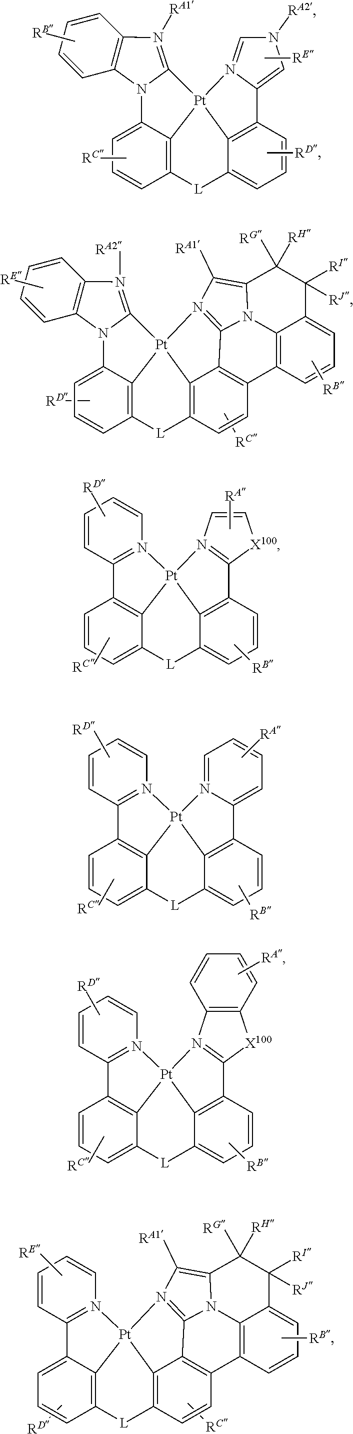

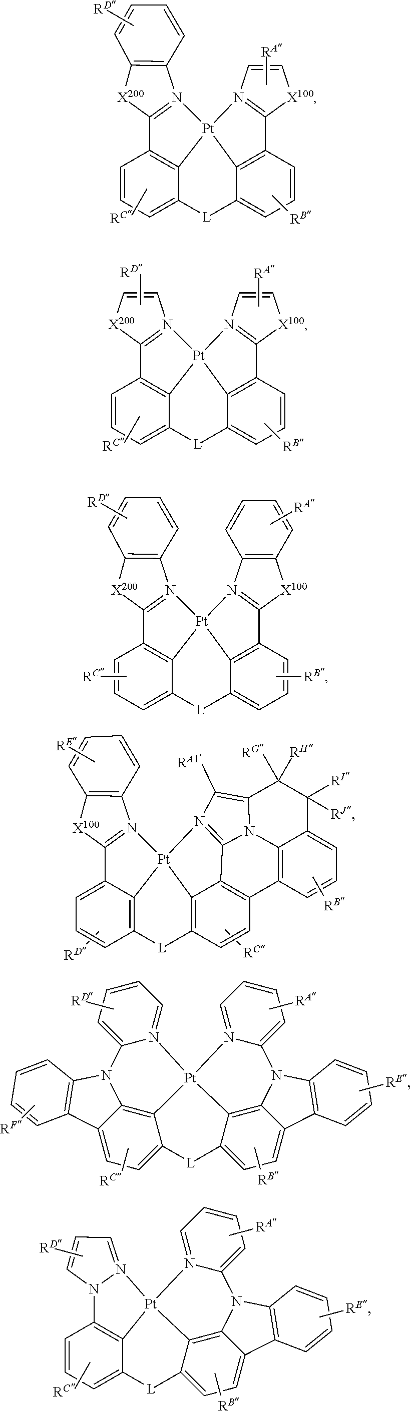

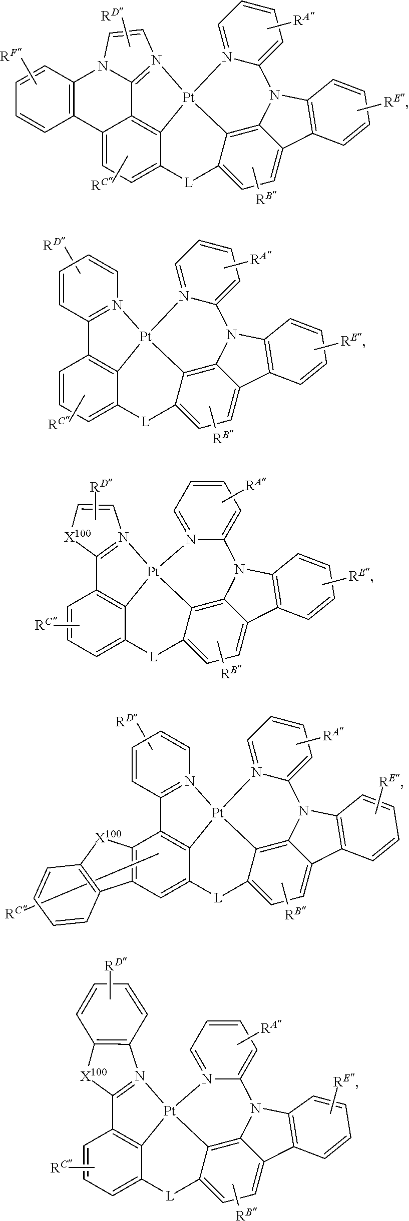

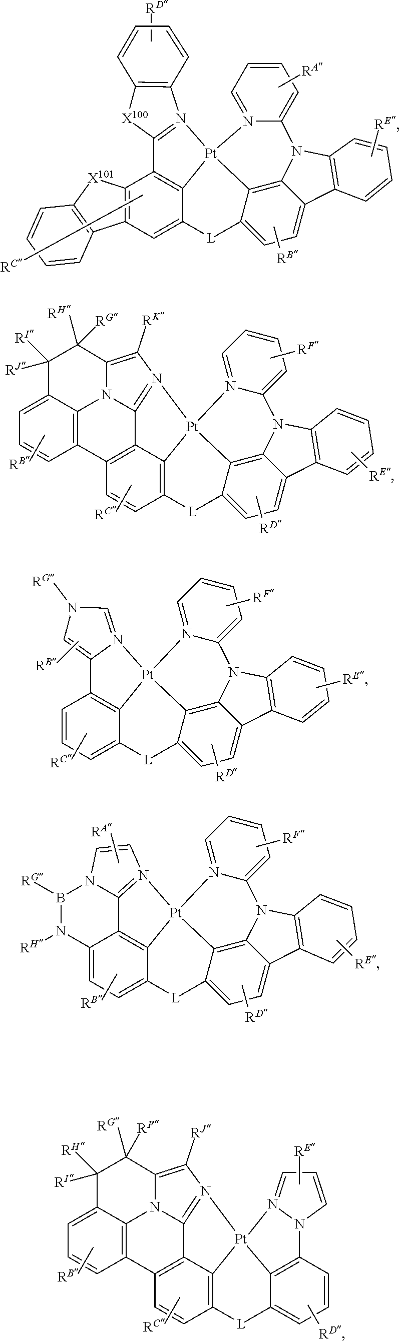

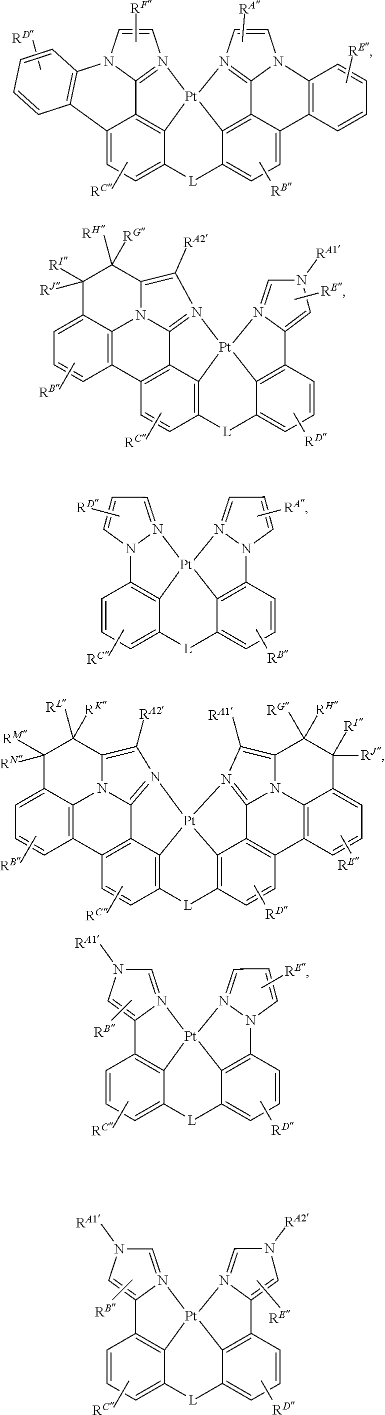

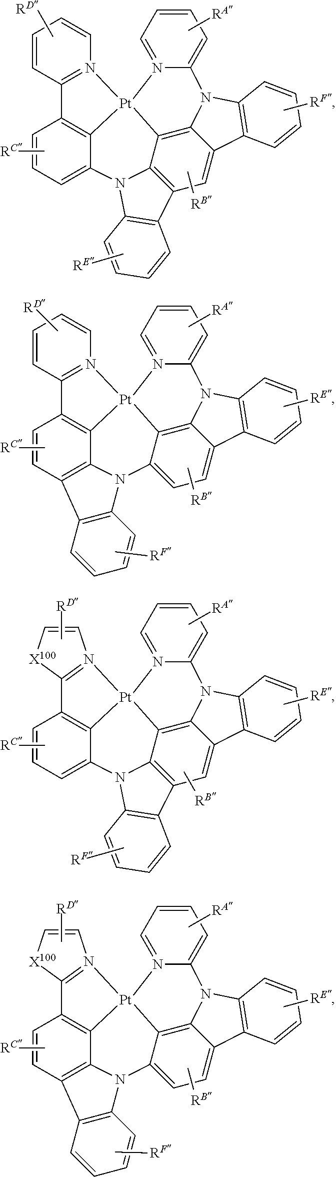

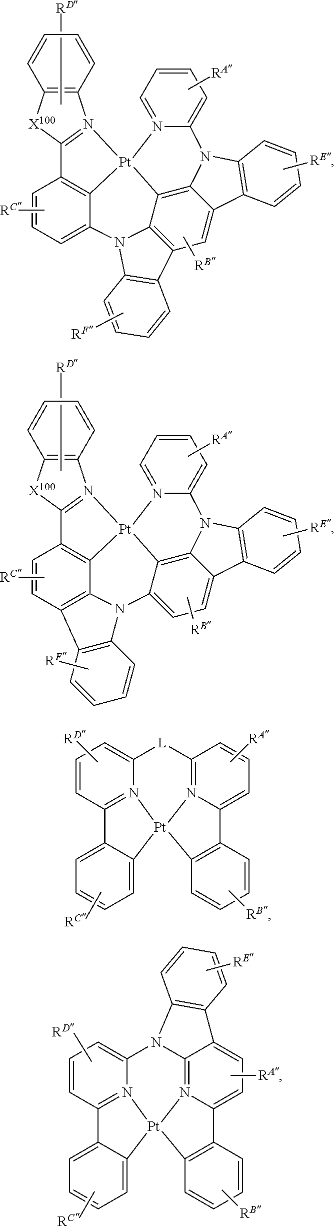

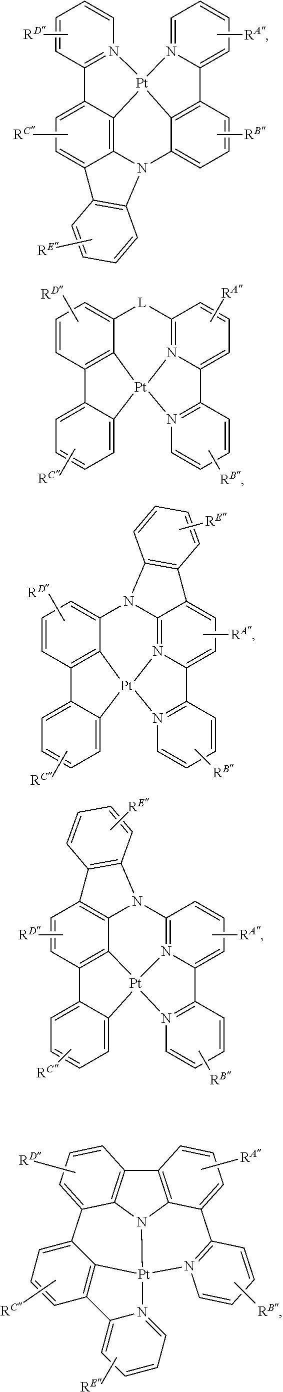

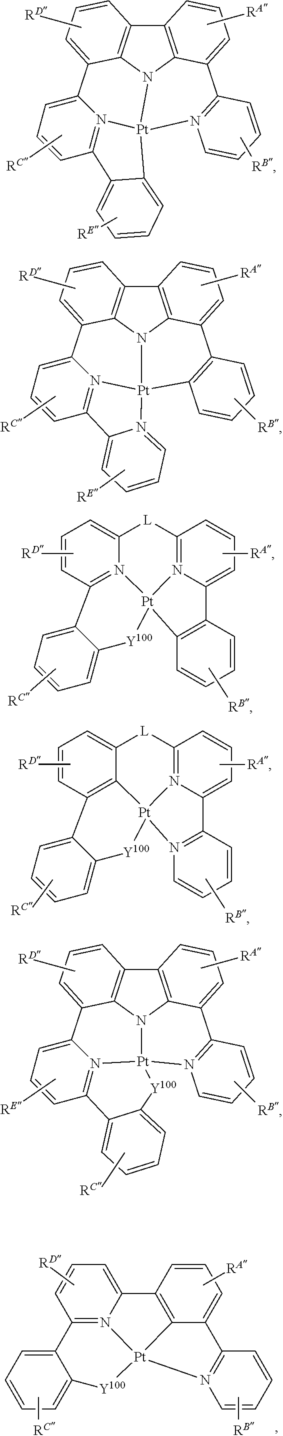

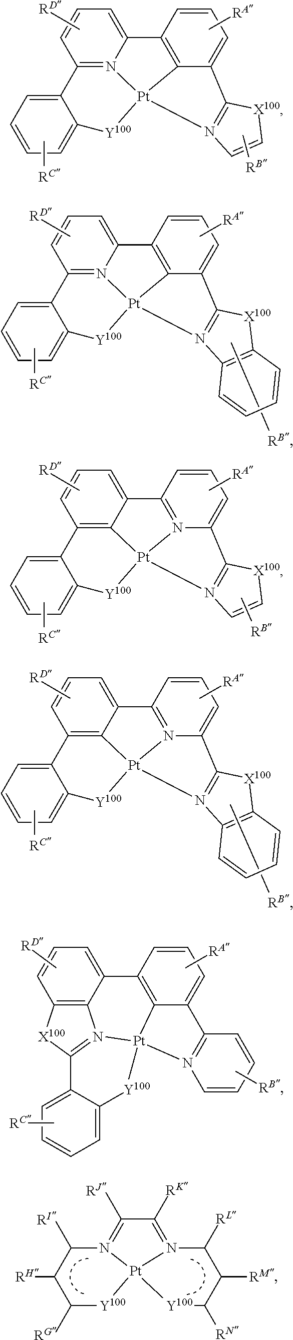

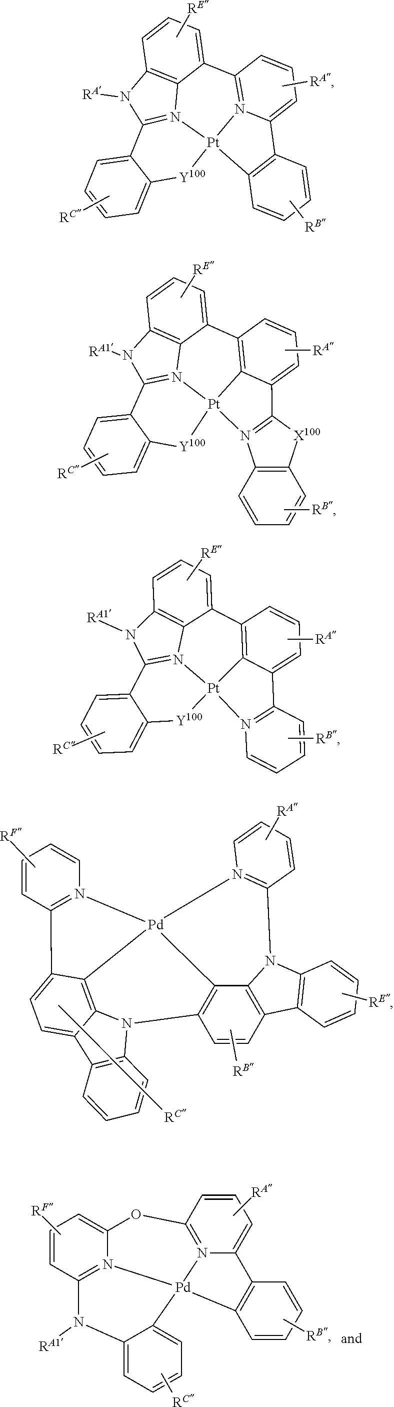

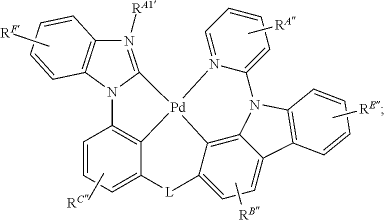

each of X⁹⁶ to X⁹⁹ is independently C or N;
each Y¹⁰⁰ is independently selected from the group consisting of a NR″, O, S, and Se;
L is independently selected from the group consisting of a direct bond, BR″, BR″R′″, NR″, PR″, O, S, Se, C═O, C═S, C═Se, C═NR″, C═CR″R′″, S═O, SO₂, CR″, CR″R′″, SiR″R′″, GeR″R′″, alkyl, cycloalkyl, aryl, heteroaryl, and combinations thereof; X¹⁰⁰, X²⁰⁰ for each occurrence is selected from the group consisting of O, S, Se, NR″, and CR″R′″; each R^{10a}, R^{20a}, R^{30a}, R^{40a}, and R^{50a}, R^{A″}, R^{B″}, R^{C″}, R^{D″}, R^{E″}, and R^{F″} independently represents mono-, up to the maximum substitutions, or no substitutions; each of R, R′, R″, R′″, R^{10a}, R^{11a}, R^{12a}, R^{13a}, R^{20a}, R^{30a}, R^{40a}, R^{50a}, R⁶⁰, R⁷⁰, R⁹⁷, R⁹⁸, R⁹⁹, R^{A1′}, R^{A2′}, R^{A″}, R^{B″}, R^{C″}, R^{D″}, R^{E″}, R^{F″}, R^{G″}, R^{H″}, R^{I″}, R^{J″}, R^{K″}, R^{L″}, R^{M″}, and R^{N″} is independently a hydrogen or a substituent selected from the group consisting of the General Substituents as defined herein; wherein any two substituents can be fused or joined to form into a ring.

In some embodiments of the OLED where the sensitizer is selected from the group consisting of the structures in the SENSITIZER LIST, one or more of R, R′, R″, R′″, R^{10a}, R^{11a}, R^{12a}, R^{13a}, R^{20a}, R^{30a}, R^{40a}, R⁵⁰, R⁶⁰, R, R⁹⁷, R⁹⁸, R⁹⁹, R^{A1′}, R^{A2′}, R^{A″}, R^{B″}, R^{C″}, R^{D″}, R^{E″}, R^{F″}, R^{G″}, R^{H″}, R^{I″}, R^{J″}, R^{K″}, R^{L″}, R^{M″}, and R^{N″} comprises a moiety selected from the group consisting of fully or partially deuterated aryl, fully or partially deuterated alkyl, boryl, silyl, germyl, 2,6-terphenyl, 2-biphenyl, 2-(tert-butyl)phenyl, tetraphenylene, tetrahydronaphthalene, and combinations thereof.

In some embodiments, the sensitizer and/or the acceptor can be a phosphorescent or fluorescent material/emitter. Phosphorescence generally refers to emission of a photon with a change in electron spin quantum number, i.e., the initial and final states of the emission have different electron spin quantum numbers, such as from T₁ to S₀ state. Most of the Ir and Pt complexes currently used in OLED are phosphorescent emitters. In some embodiments, if an exciplex formation involves a triplet emitter, such exciplex can also emit phosphorescent light. On the other hand, fluorescent emitters generally refer to emission of a photon without a change in electron spin quantum number, such as from S₁ to S₀ state, or from D₁ to D₀ state. Fluorescent emitters can be delayed fluorescent or non-delayed fluorescent emitters. Depending on the spin state, fluorescent emitter can be a singlet emitter or a doublet emitter, or other multiplet emitter. It is believed that the internal quantum efficiency (IQE) of fluorescent OLEDs can exceed the 25% spin statistics limit through delayed fluorescence. There are two types of delayed fluorescence, i.e. P-type and E-type delayed fluorescence. P-type delayed fluorescence is generated from triplet-triplet annihilation (TTA). On the other hand, E-type delayed fluorescence does not rely on the collision of two triplets, but rather on the thermal population between the triplet states and the singlet excited states. Thermal energy can activate the transition from the triplet state back to the singlet state. This type of delayed fluorescence is also known as thermally activated delayed fluorescence (TADF). E-type delayed fluorescence characteristics can be found in an exciplex system or in a single compound. Without being bound by theory, it is believed that TADF requires a compound or an exciplex having a small singlet-triplet energy gap (ΔE_{S-T}) less than or equal to 400, 350, 300, 250, 200, 150, 100, or 50 meV. There are two major types of TADF emitters, one is called donor-acceptor type TADF, the other one is called multiple resonance (MR) TADF. Often, donor-acceptor single compounds are constructed by connecting an electron donor moiety such as amino- or carbazole-derivatives and an electron acceptor moiety such as N-containing six-membered aromatic rings or cyano-substituted aromatic rings. Donor-acceptor exciplex can be formed between a hole transporting compound and an electron transporting compound. The examples for MR-TADF include highly conjugated fused ring systems. In some embodiments, MR-TADF materials comprise boron, carbon, and nitrogen atoms. They may comprise other atoms as well, for example oxygen. In some embodiments, the reverse intersystem crossing time from T₁ to S₁ of the delayed fluorescent emission at 293K is less than or equal to 10 microseconds. In some embodiments, such time can be greater than 10 microseconds and less than 100 microseconds.

In some embodiments of the OLED, at least one of the following conditions is true:
(1) the sensitizer compound is capable of functioning as a TADF emitter in an OLED at room temperature;
(2) the acceptor compound is a delayed-fluorescent compound functioning as a TADF emitter in the OLED at room temperature.

In some embodiments of the OLED, the delayed fluorescence material comprises at least one donor group and at least one acceptor group. In some embodiments, the delayed fluorescence material is a metal complex. In some embodiments, the delayed fluorescence material is a non-metal complex. In some embodiments, the delayed fluorescence material is a Pt, Pd, Zn, Cu, Ag, or Au complex (some of them are also called metal-assisted (MA) TADF). In some embodiments, the metal-assisted delayed fluorescence material comprises a metal-carbene bond. In some embodiments, the non-delayed fluorescence material or delayed fluorescence material comprises at least one chemical group selected from the group consisting of aryl-amine, aryloxy, arylthio, triphenylene, carbazole, indolocarbazole, dibenzothiophene, dibenzofuran, dibenzoselenophene, 5λ²-benzo[d]benzo[4,5]imidazo[3,2-a]imidazole, 5,9-dioxa-13b-boranaphtho[3,2,1-de]anthracene, 5λ²,9λ²-diaza-13b-boranaphtho[2,3,4-de]anthracene, 5-oxa-9λ²-aza-13b-boranaphtho[3,2,1-de]anthracene, azaborinine, oxaborinine, dihydroacridine, xanthene, dihydrobenzoazasiline, dibenzooxasiline, phenoxazine, phenoxathiine, phenothiazine, dihydrophenazine, fluorene, naphthalene, anthracene, phenanthrene, phenanthroline, benzoquinoline, quinoline, isoquinoline, quinazoline, pyrimidine, pyrazine, pyridine, triazine, boryl, amino, silyl, aza-variants thereof, and combinations thereof. In some embodiments, non-delayed the fluorescence material or delayed fluorescence material comprises a tri(aryl/heteroaryl)borane with one or more pairs of the substituents from the aryl/heteroaryl being joined to form a ring. In some embodiments, the fluorescence material comprises at least one chemical group selected from the group consisting of naphthalene, anthracene, phenalene, phenanthrene, fluorene, pyrene, chrysene, perylene, and azulene.

In some embodiments of the OLED, the TADF material has the formula of M(L⁵)(L⁶), wherein M is Cu, Ag, or Au, L⁵ and L⁶ are different, and L⁵ and L⁶ are independently selected from the group consisting of:

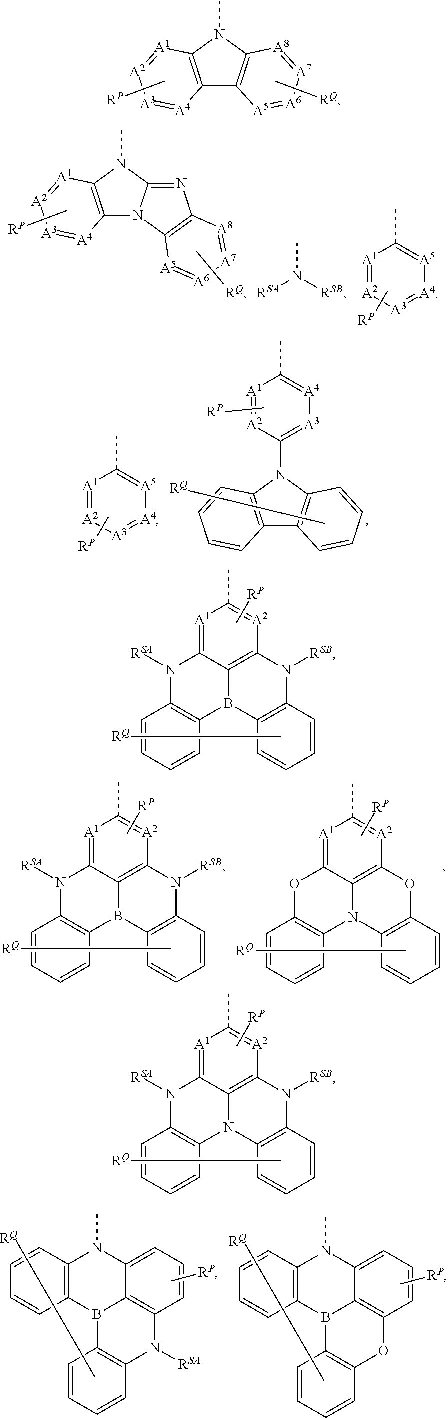

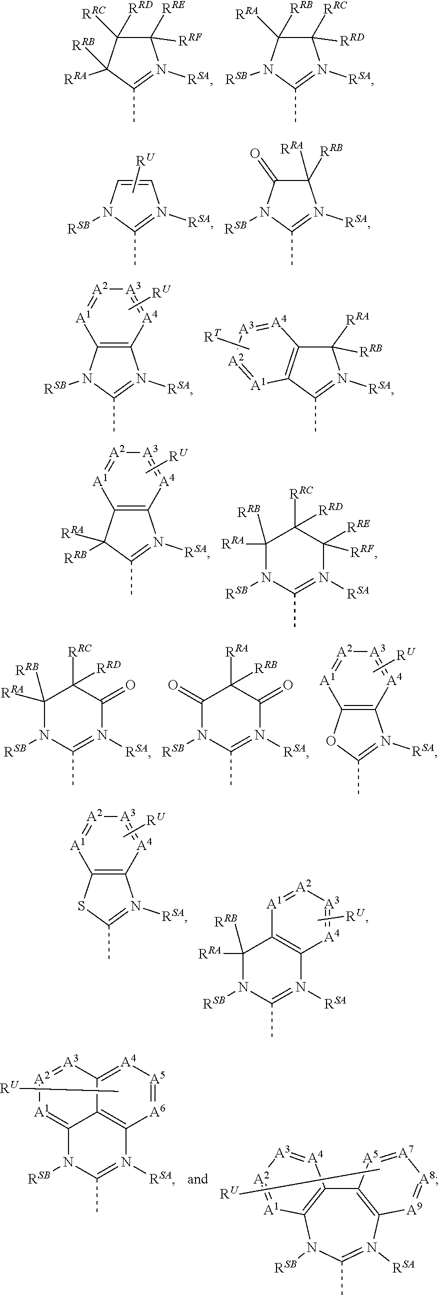

wherein A¹-A⁹ are each independently selected from C or N;
each R^{P}, R^{Q}, R^{T}, and R^{U} independently represents mono-, up to the maximum substitutions, or no substitutions; wherein each R^{P}, R^{Q}, R^{T}, R^{U}, R^{SA}, R^{SB}, R^{RA}, R^{RB}, R^{RC}, R^{RD}, R^{RE}, and R^{RF} is independently a hydrogen or a substituent selected from the group consisting of the general substituents as defined herein; any two substituents can be joined or fused to form a ring.

In some embodiments of the OLED, the TADF material is selected from the group consisting of:

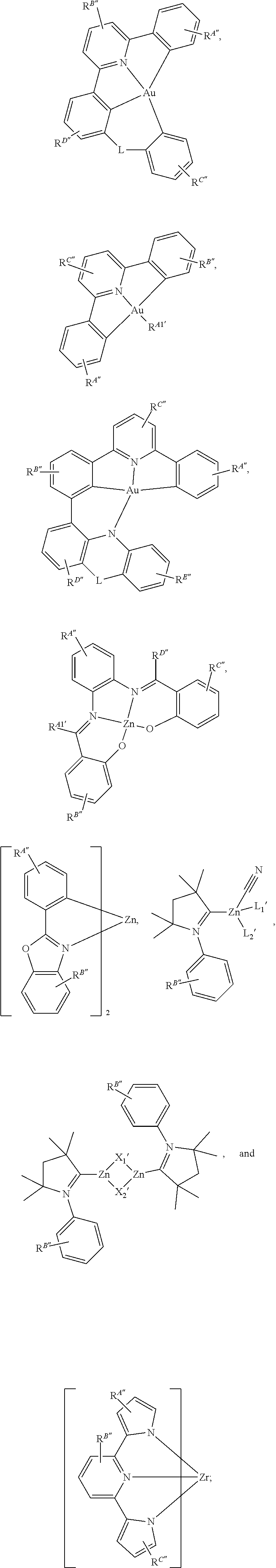

wherein each R^{A″}, R^{B″}, R^{C″}, R^{D″}, R^{E″}, and R^{F″} can independently represent from mono to the maximum possible number of substitutions, or no substitution;
   each R″, R′″, R^{A}, R^{A″}, R^{B″}, R^{C″}, R^{D″}, R^{E″}, and R^{F″} is independently a hydrogen or a substituent selected from the group consisting of the general substituents as defined herein; wherein any two substituents can be fused or joined to form into a ring.
wherein L is independently selected from the group consisting of a direct bond, BR″, BR″R′″, NR″, PR″, O, S, Se, C═O, C═S, C═Se, C═NR″, C═CR″R′″, S═O, SO₂, CR″, CR″R′″, SiR″R′″, GeR″R′″, alkyl, cycloalkyl, aryl, heteroaryl, and combinations thereof;
wherein each of L₁′ and L₂′ is a monodentate anionic ligand,
wherein each of X₁′ and X₂′ is a halide; and
wherein any two substituents can be fused or joined to form a ring.

In some embodiments of the OLED, the TADF material is selected from the group consisting of:

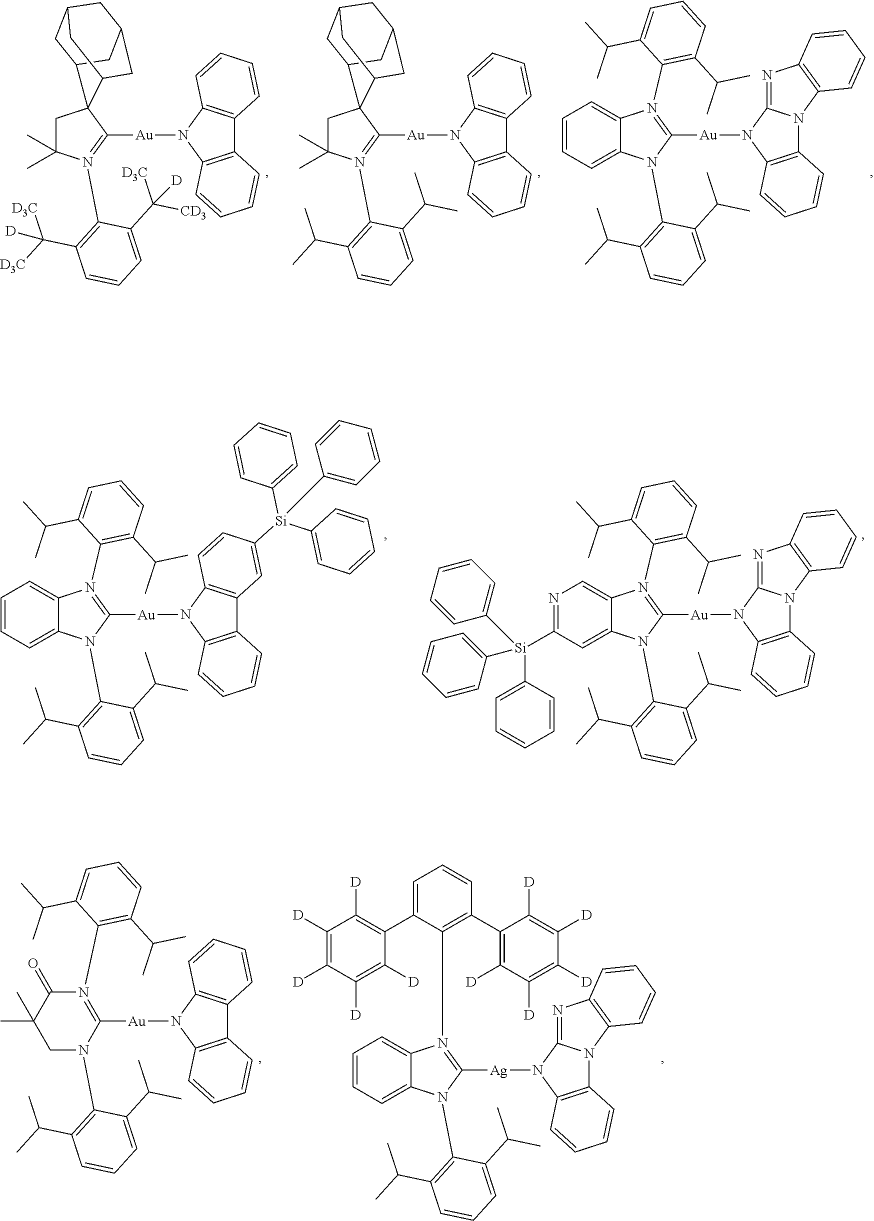

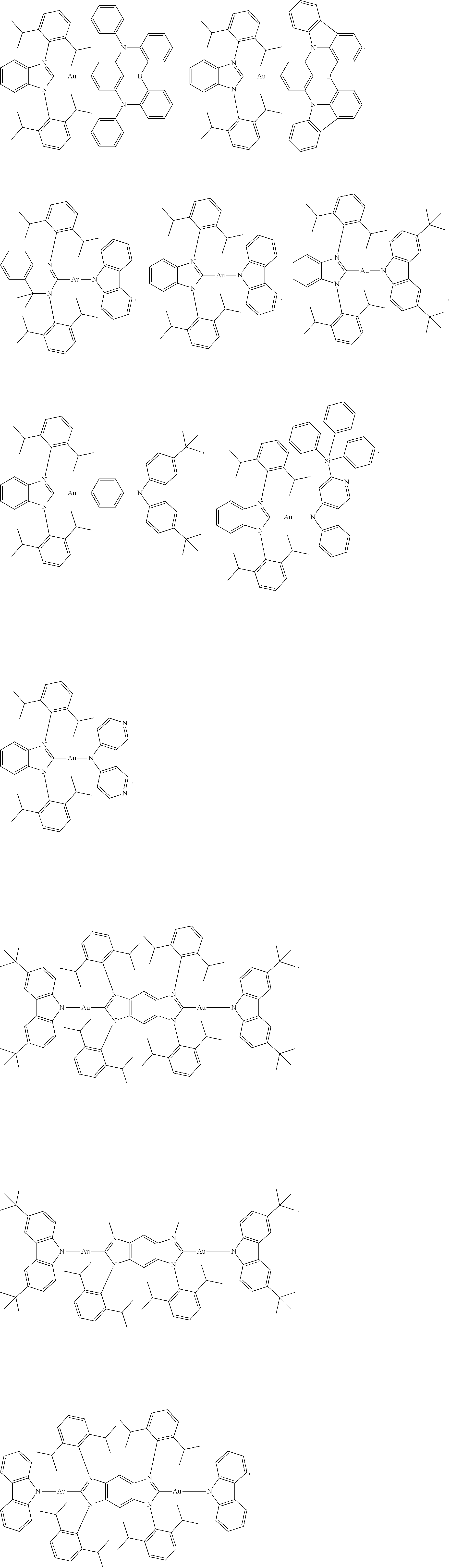

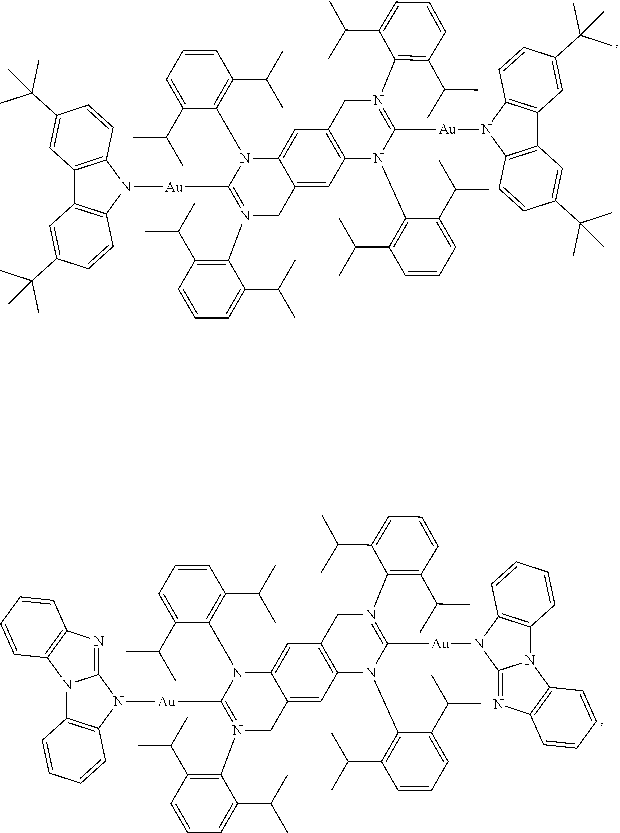

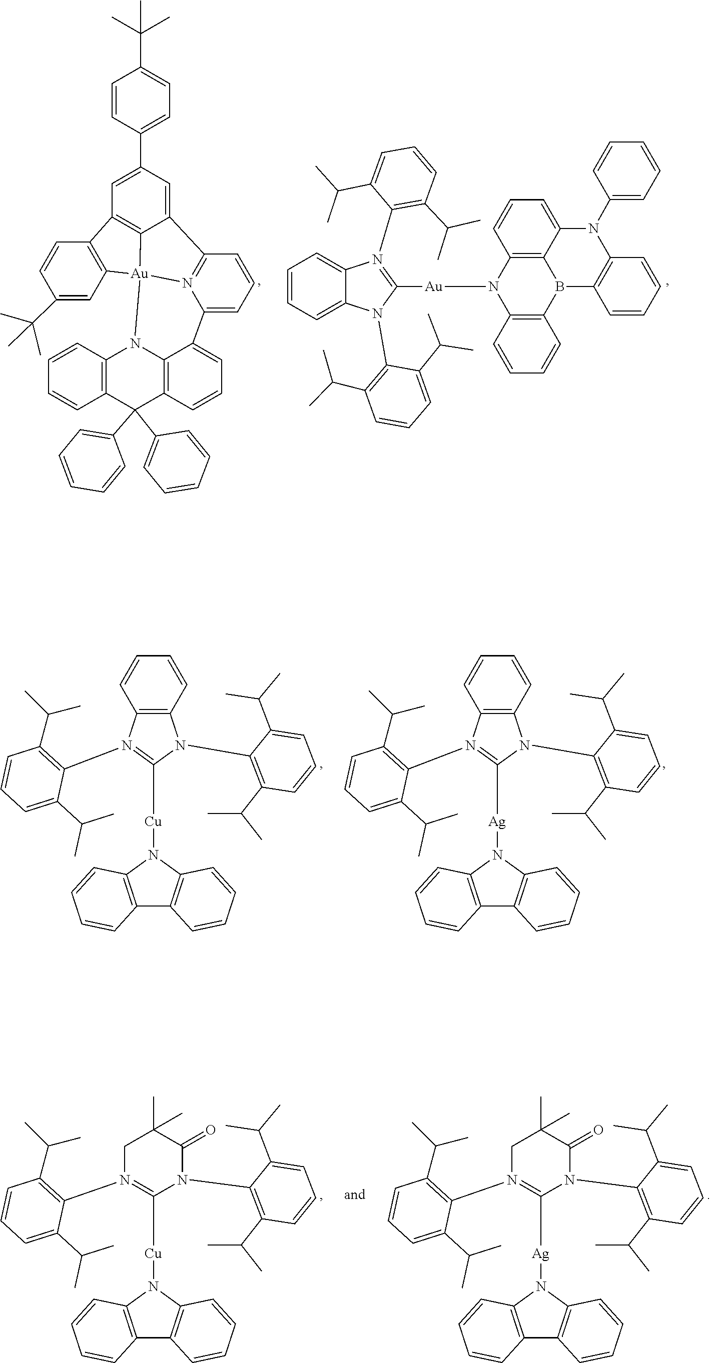

In some embodiments of the OLED, the TADF material comprises a boron atom. In some embodiments of the OLED, the TADF material comprises at least one of the donor moieties selected from the group consisting of:

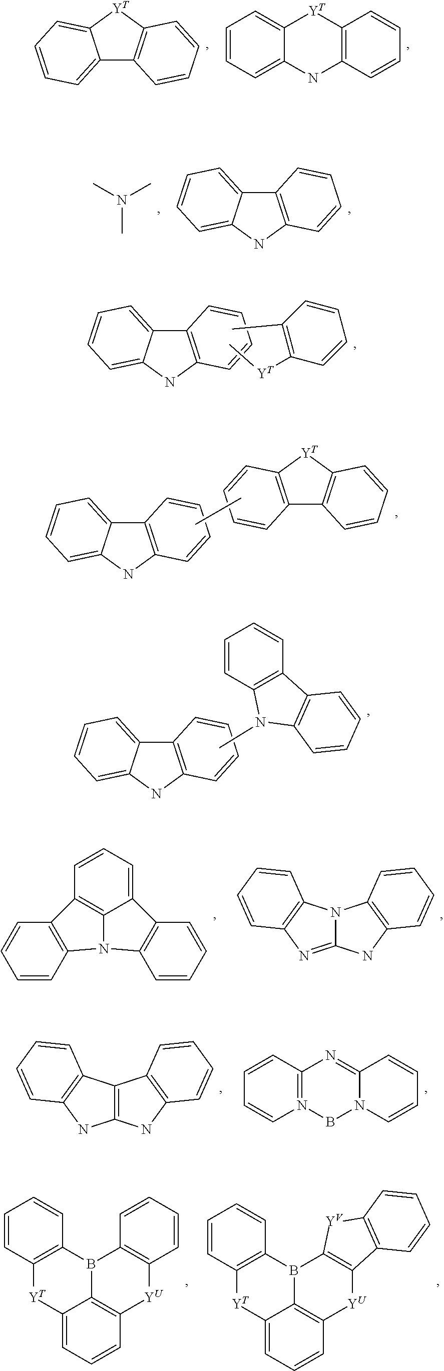

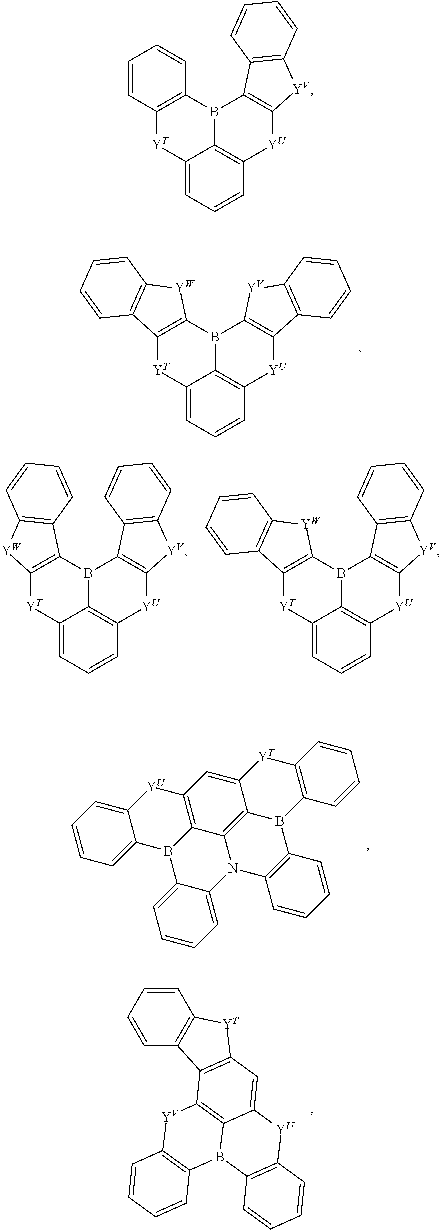

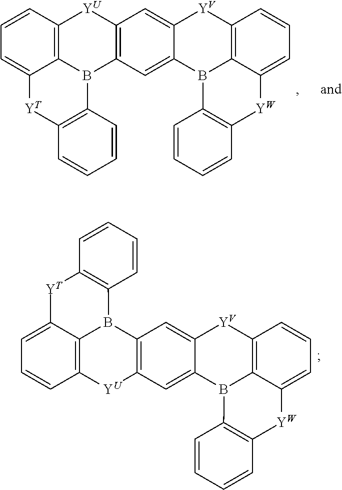

wherein Y^{T} Y^{U}, Y^{V}, and Y^{W} are each independently selected from the group consisting of B, C, Si, Ge, N, P, O, S, Se, C═O, S═O, and SO₂.

In some of the above embodiments, any carbon ring atoms up to maximum of a total number of three, together with their substituents, in each phenyl ring of any of above structures can be replaced with N.

In some embodiments, the TADF material comprises at least one of the acceptor moieties selected from the group consisting of nitrile, isonitrile, borane, fluoride, pyridine, pyrimidine, pyrazine, triazine, aza-carbazole, aza-dibenzothiophene, aza-dibenzofuran, aza-dibenzoselenophene, aza-triphenylene, imidazole, pyrazole, oxazole, thiazole, isoxazole, isothiazole, triazole, thiadiazole, and oxadiazole. In some embodiments, the acceptor moieties and the donor moieties as described herein can be connected directly, through a conjugated linker, or a non-conjugated linker, such as a sp3 carbon or silicon atom.

In some embodiments, the acceptor is a fluorescent material. In some embodiments, the fluorescent compound comprises at least one of the chemical moieties selected from the group consisting of:

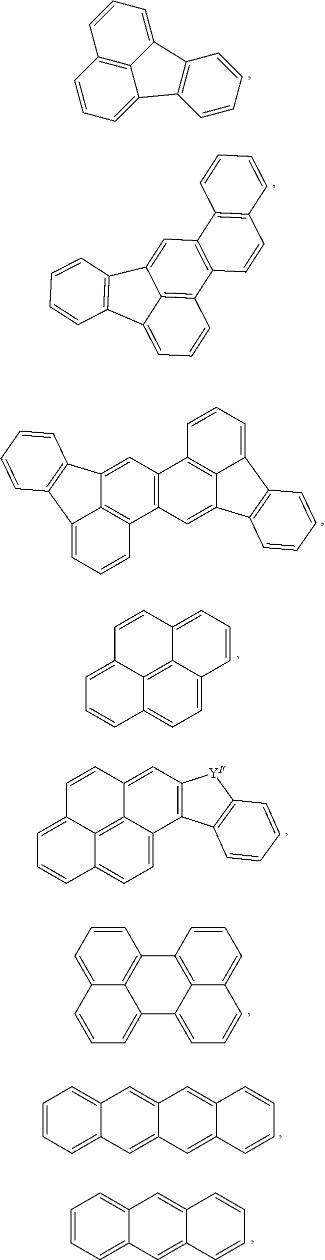

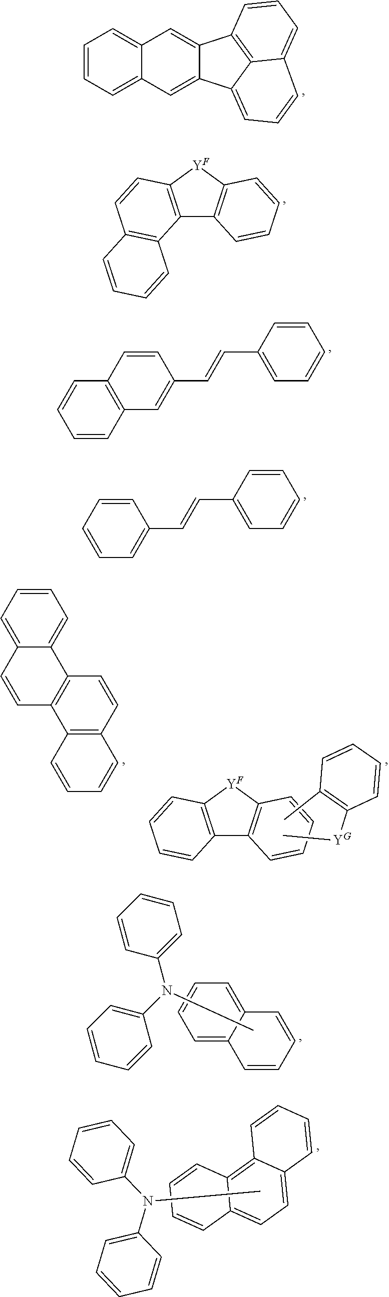

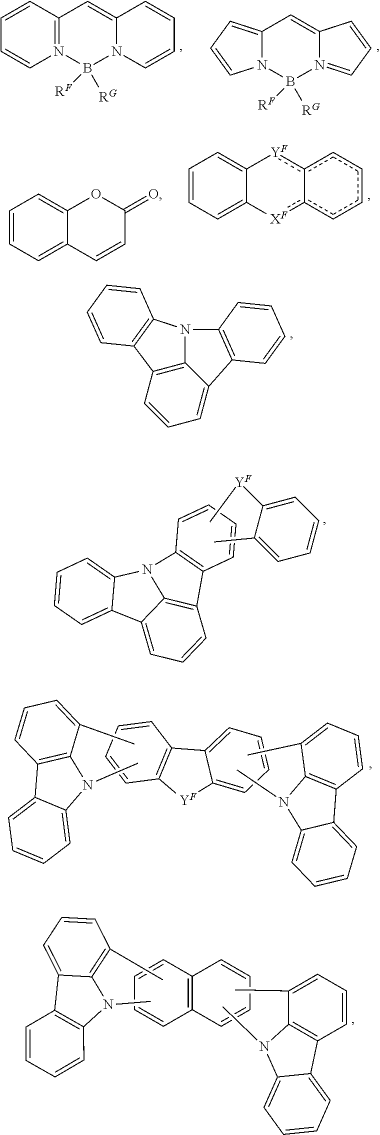

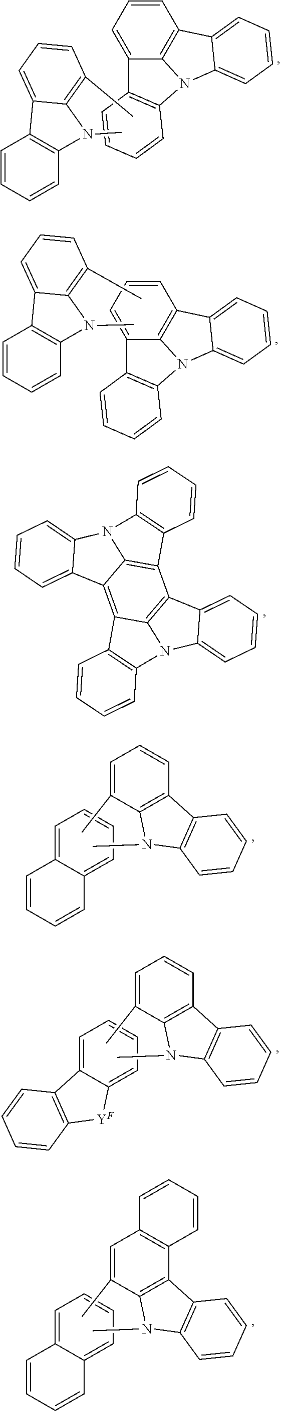

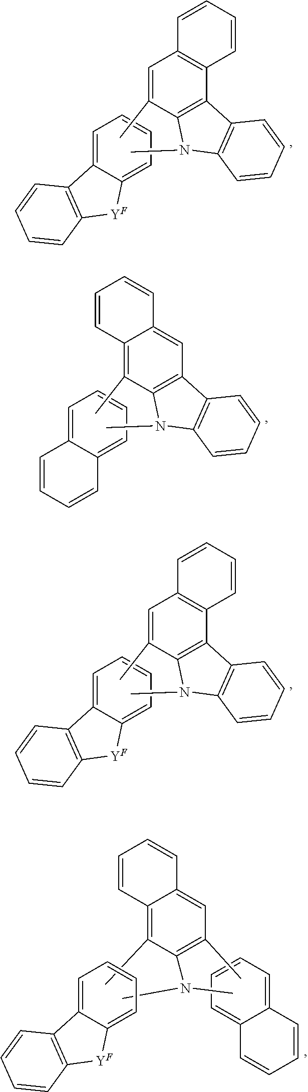

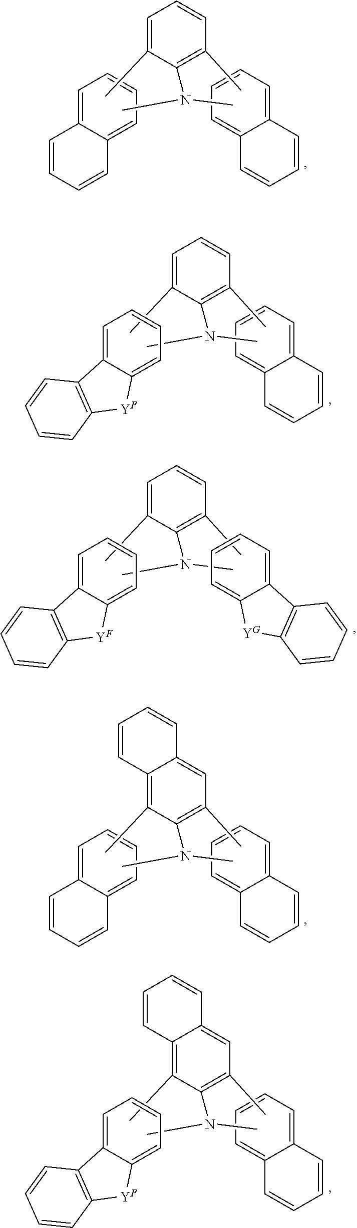

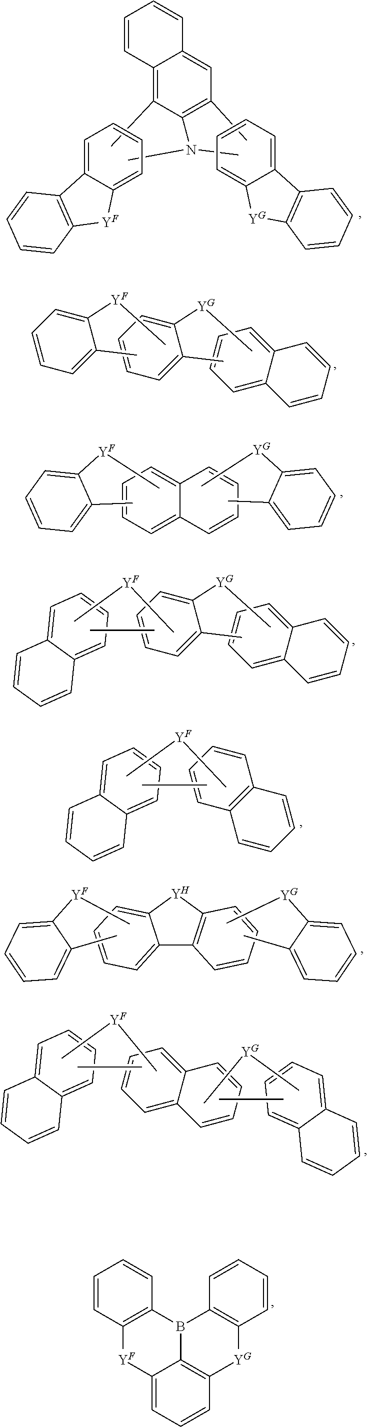

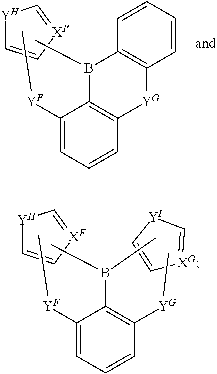

wherein Y^{F}, Y^{G}, Y^{H} and Y^{I} are each independently selected from the group consisting of B, C, Si, Ge, N, P, O, S, Se, C═O, S═O, and SO₂;
wherein X^{F} and X^{G} are each independently selected from the group consisting of C and N. In some of the above embodiments, any carbon ring atoms up to maximum of a total number of three, together with their substituents, in each phenyl ring of any of above structures can be replaced with N.

In some embodiments of the OLED, the fluorescent compound is selected from the group consisting of:

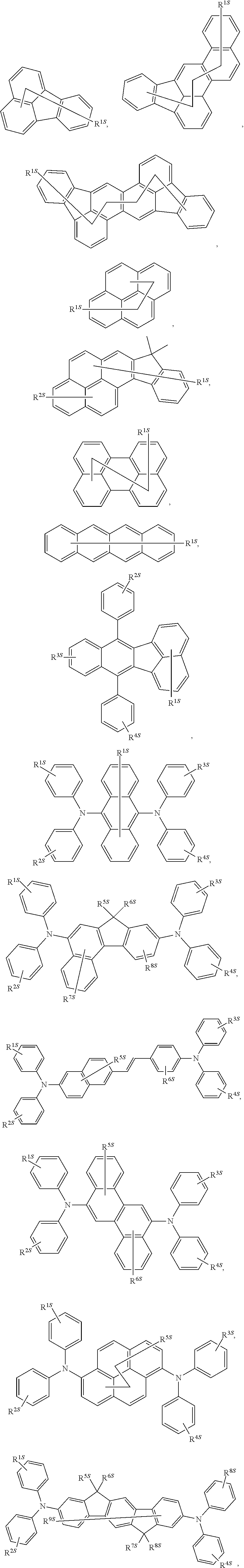

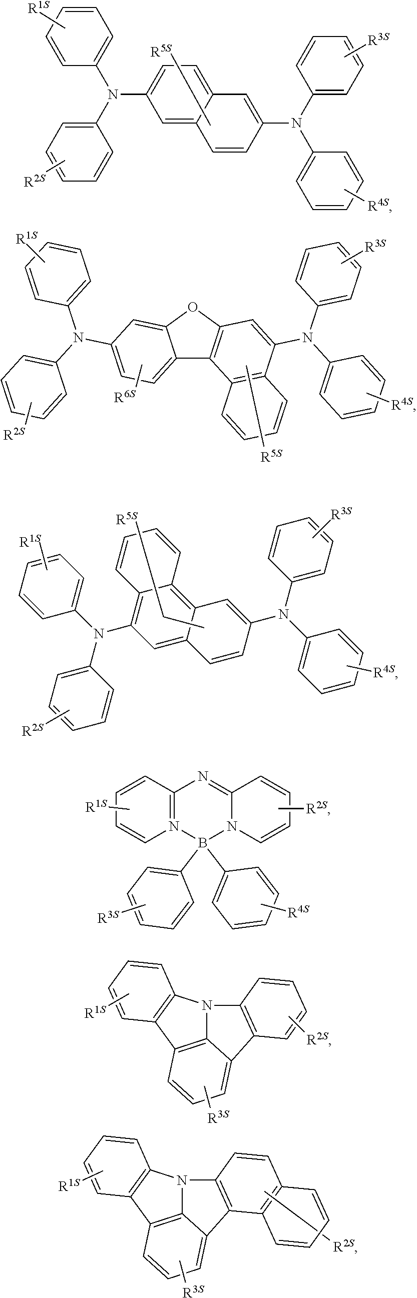

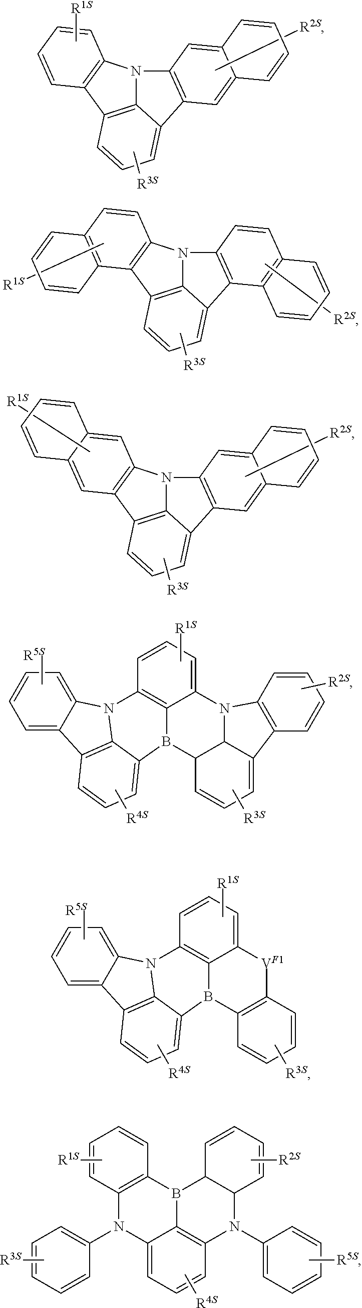

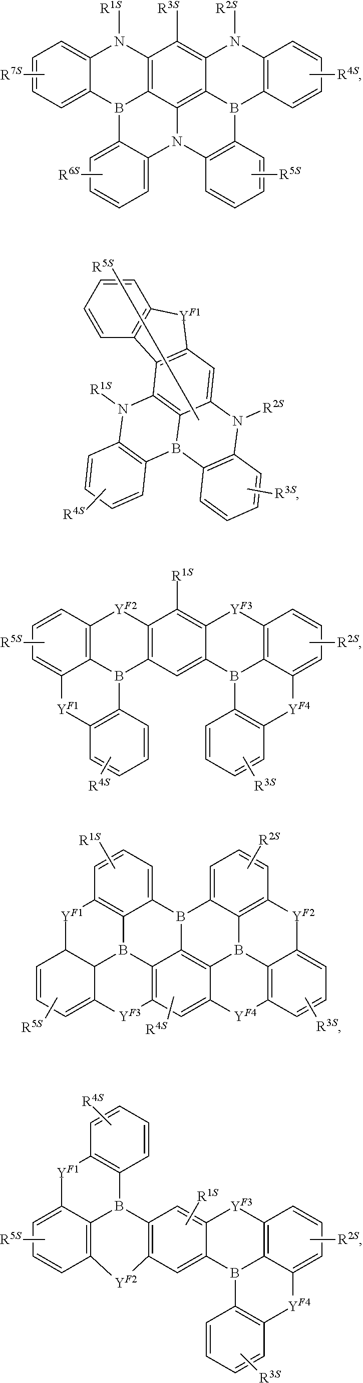

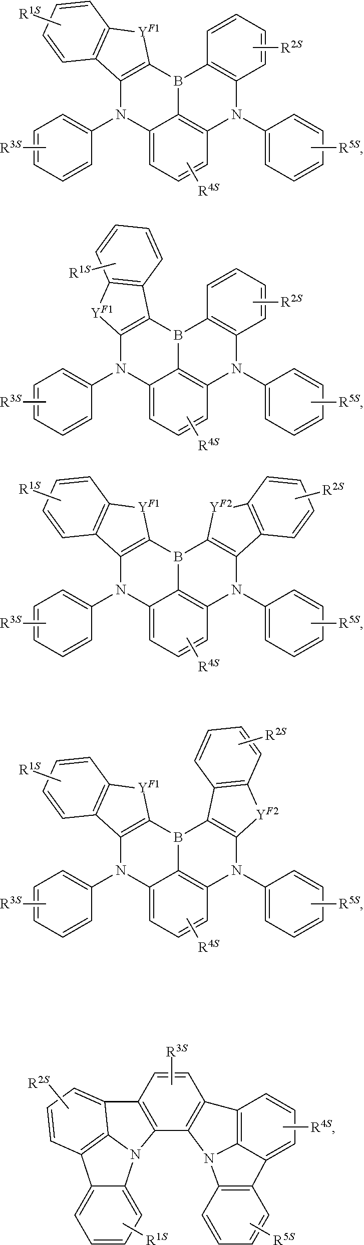

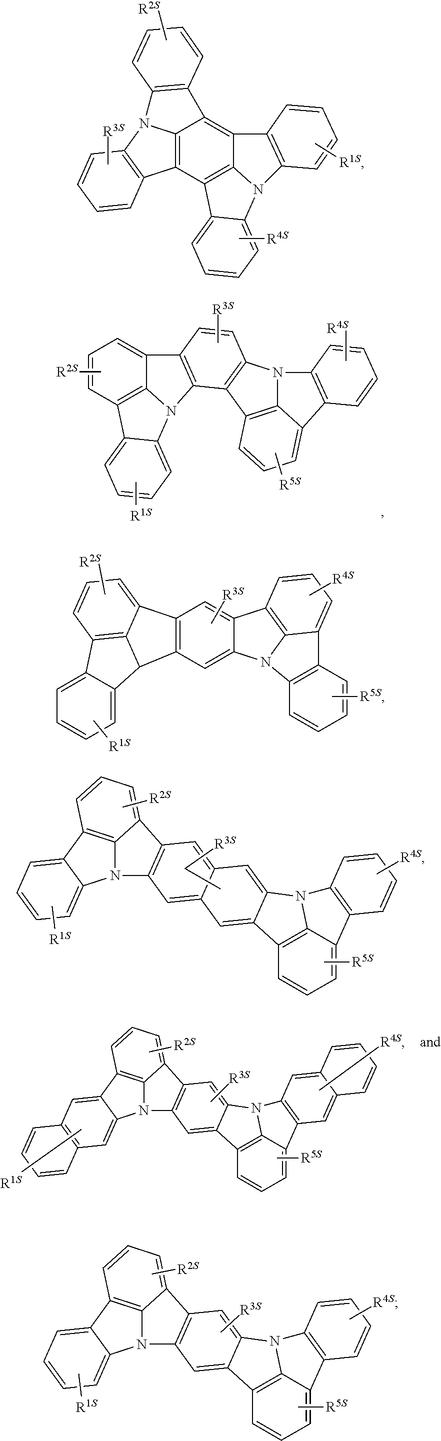

wherein Y^{F1} to Y^{F4} are each independently selected from O, S, and NR^{F1};
wherein R^{F1} and R^{1S} to R^{9S} each independently represents from mono to maximum possible number of substitutions, or no substitution; and
wherein R^{F1} and R^{1S} to R^{9S} are each independently a hydrogen or a substituent selected from the group consisting of the general substituents as defined herein, any two substituents can be joined or fused to form a ring.

In some embodiments, the acceptor compound comprises at least one moiety selected from the group consisting of:

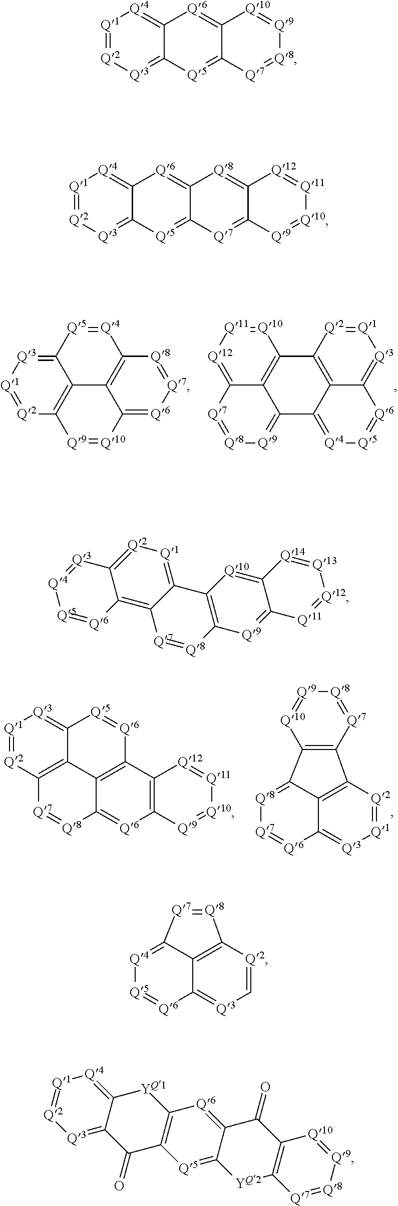

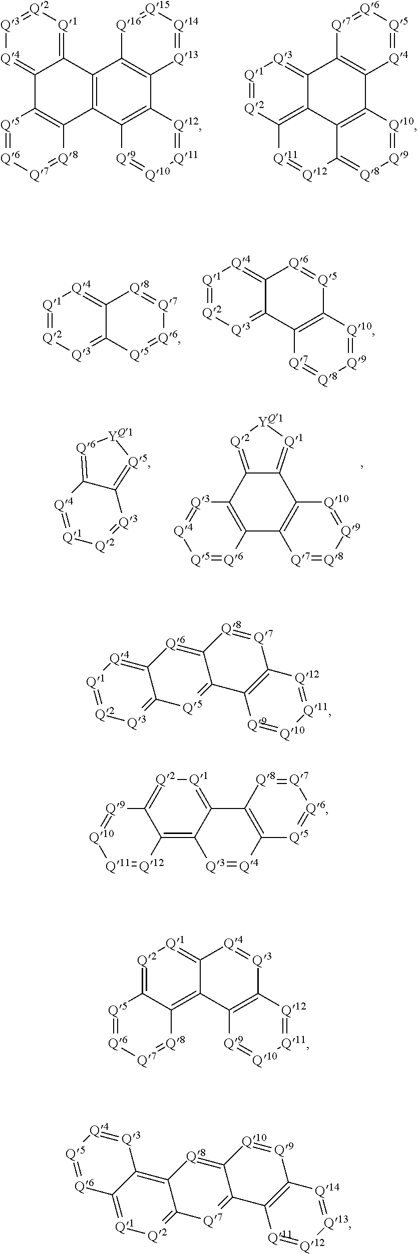

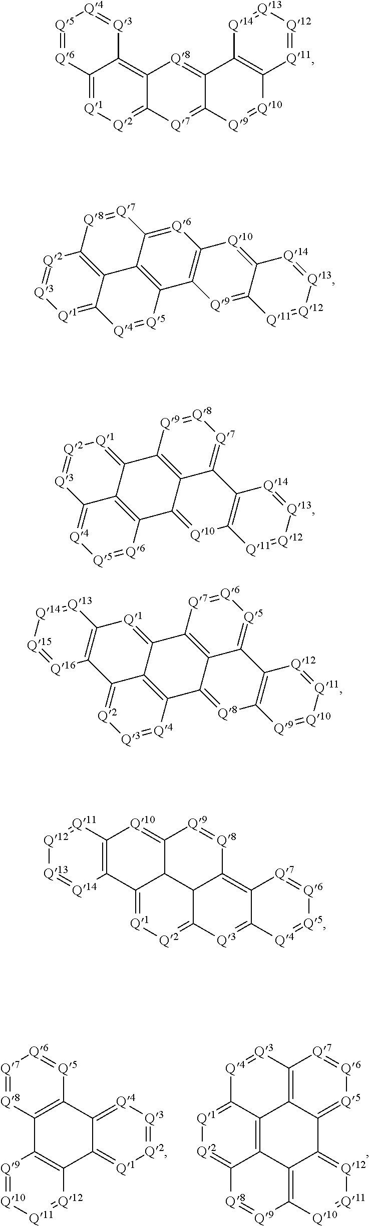

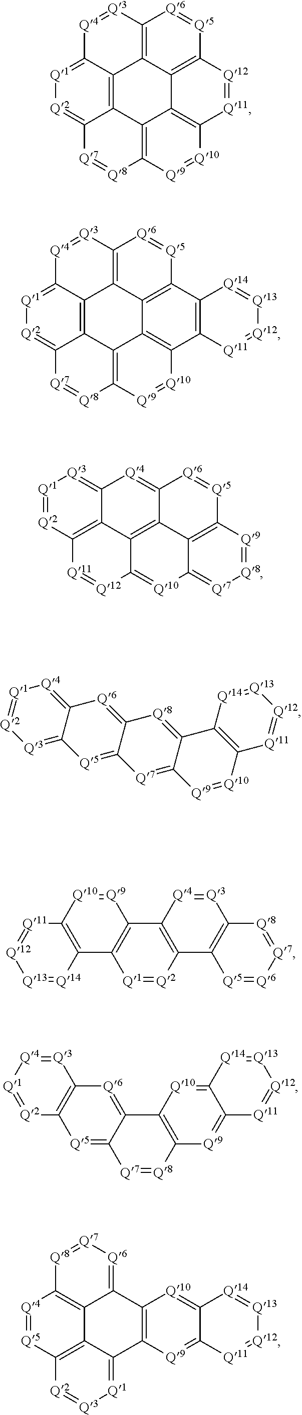

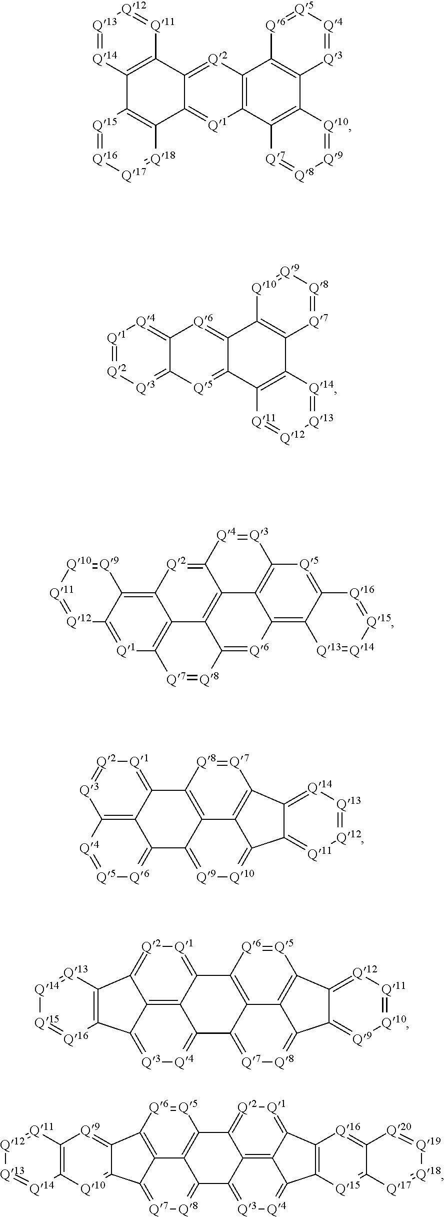

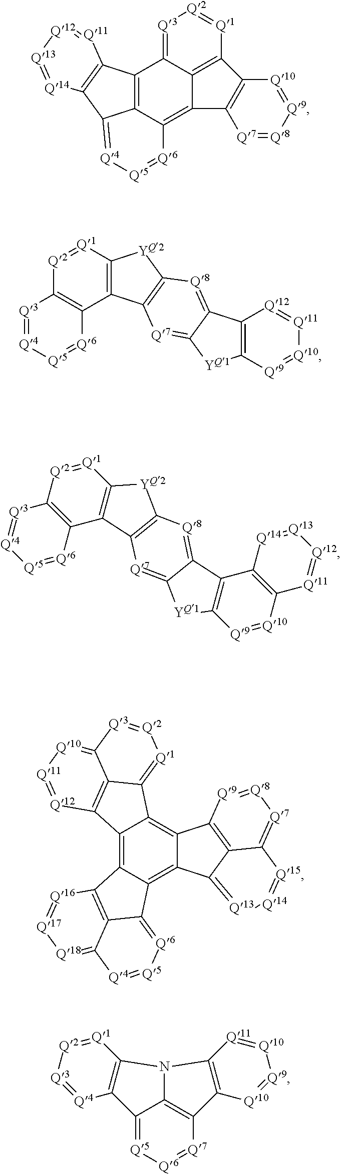

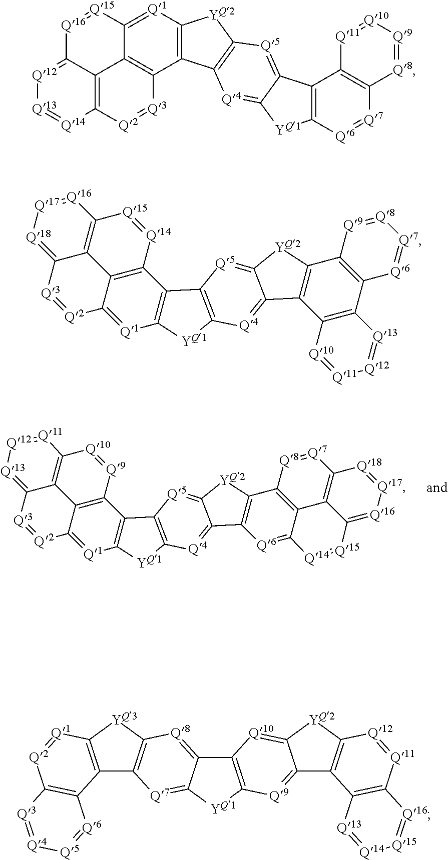

wherein each of Q′¹ to Q′¹⁸ is independently C or N; and each of Y^{Q1}, and Y^{Q2} are each independently selected from the group consisting of B, C, Si, Ge, N, P, O, S, Se, C═O, S═O, and SO₂.

In some embodiments, the acceptor compound comprises at least one moiety selected from the group consisting of:

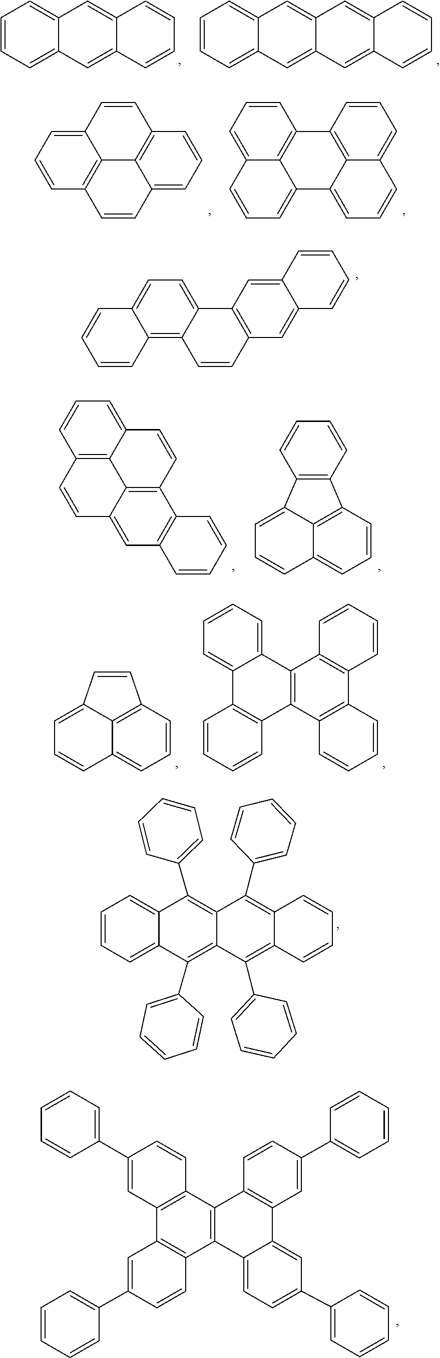

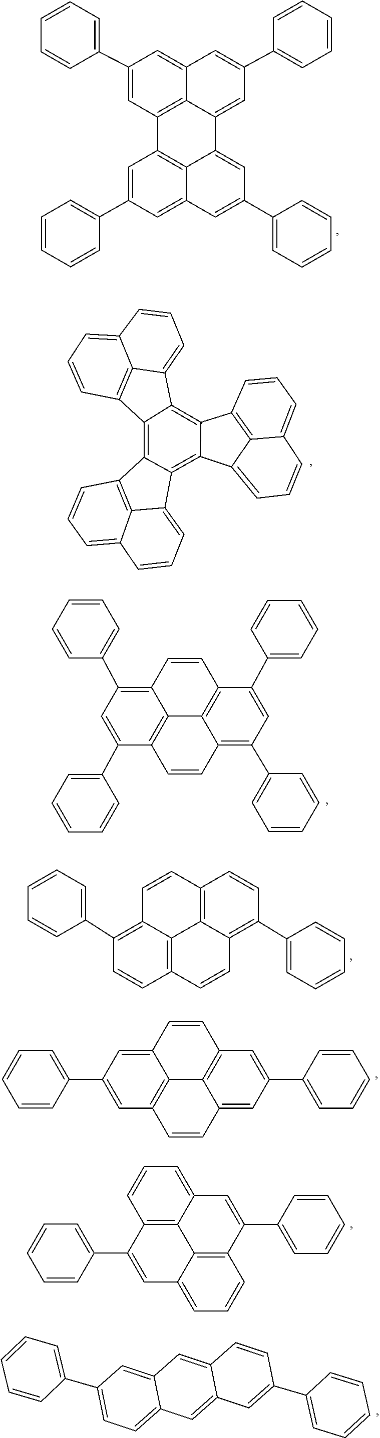

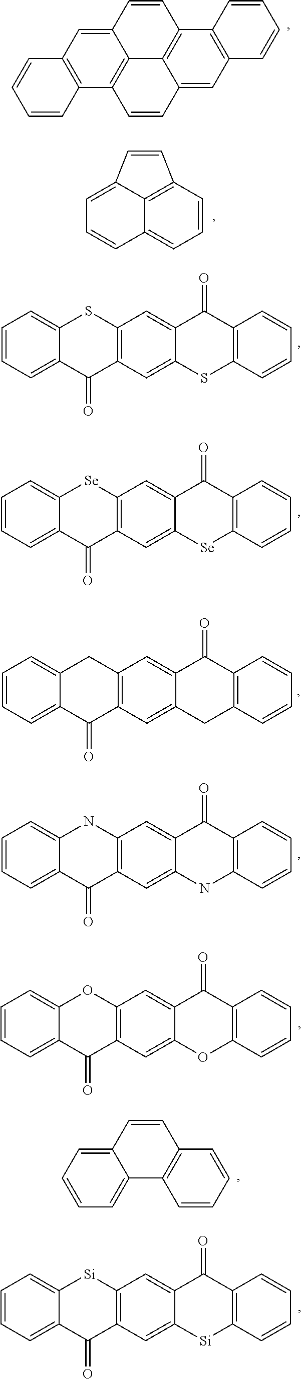

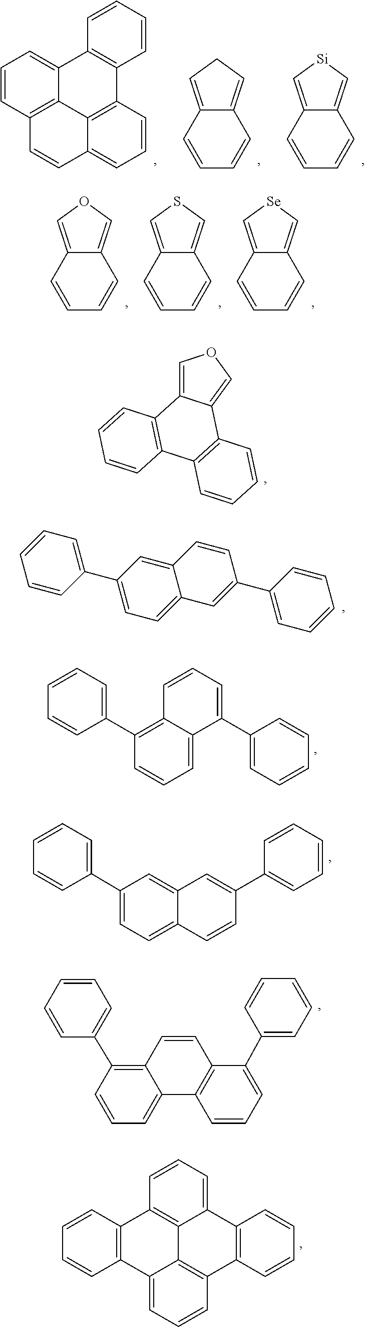

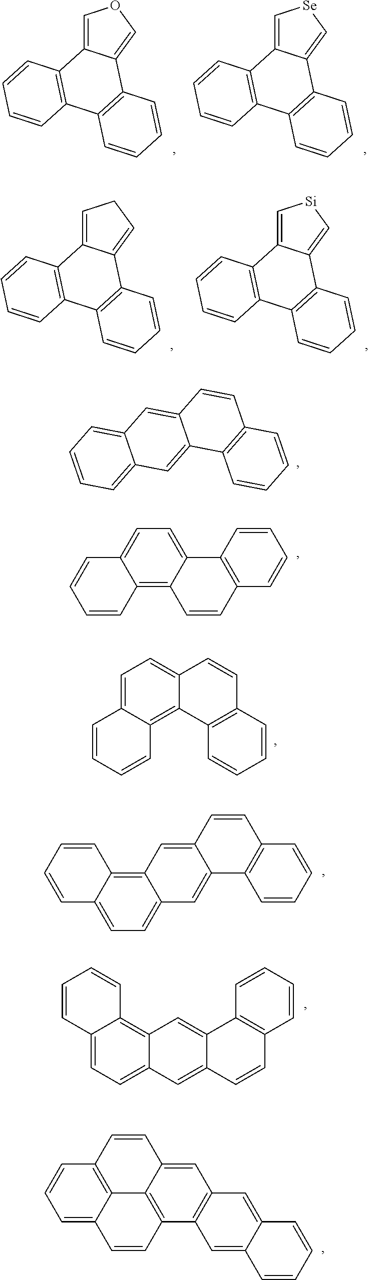

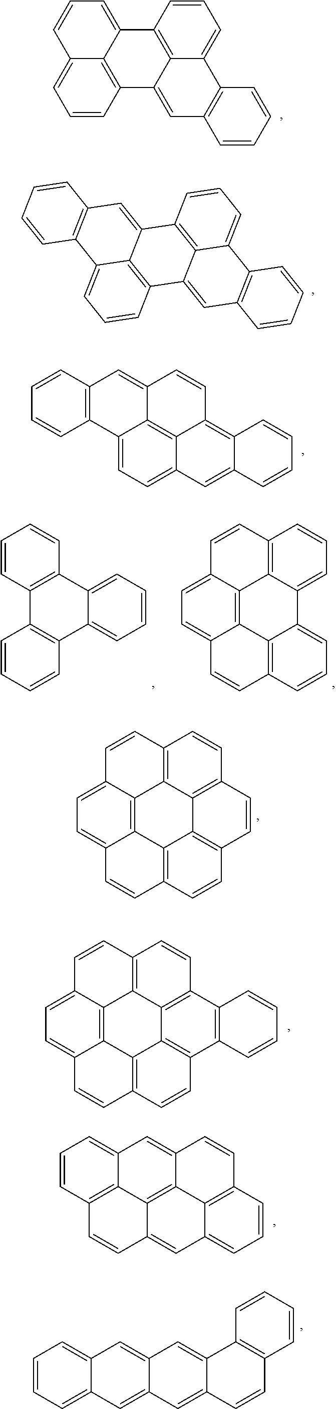

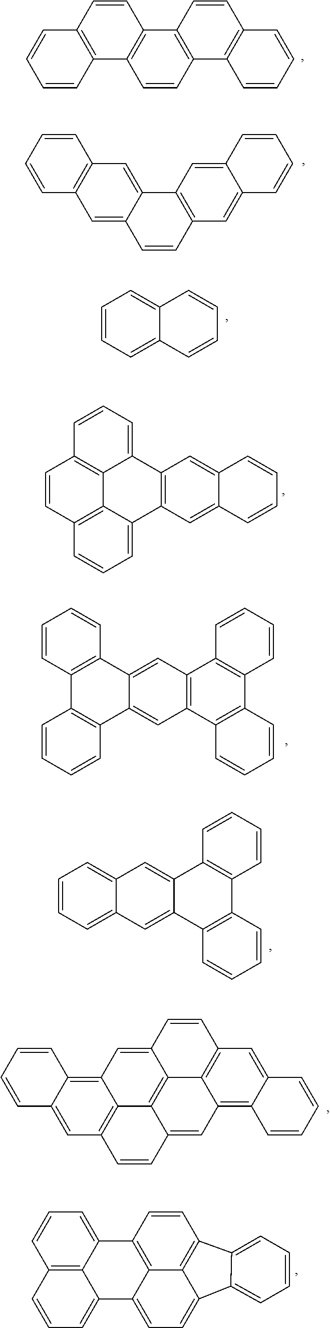

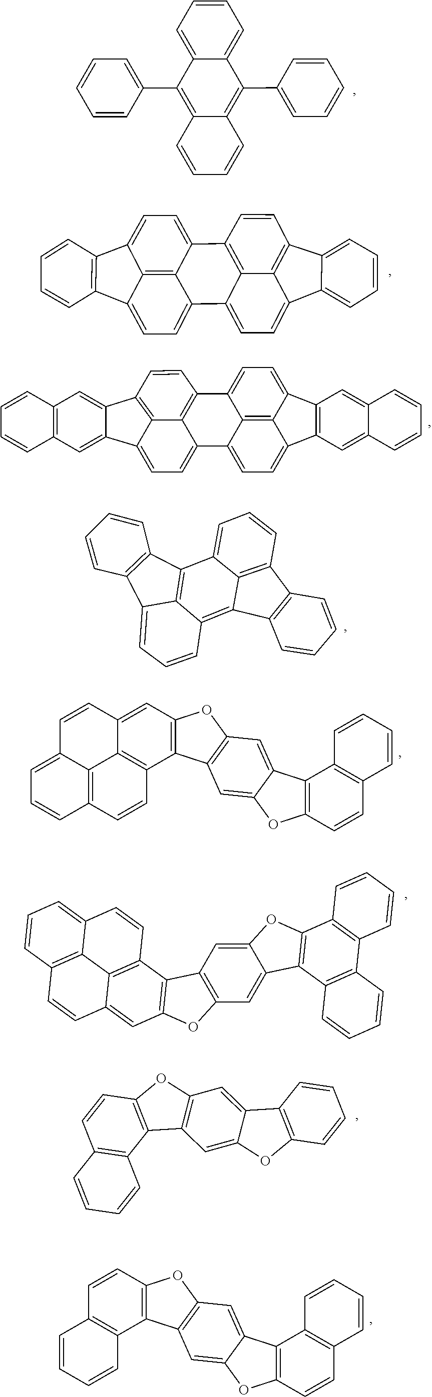

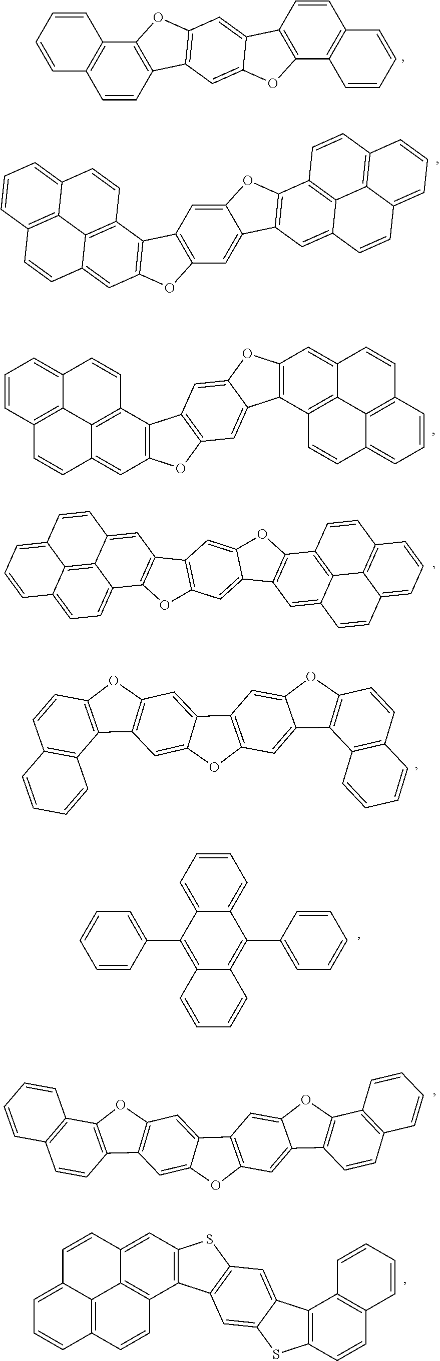

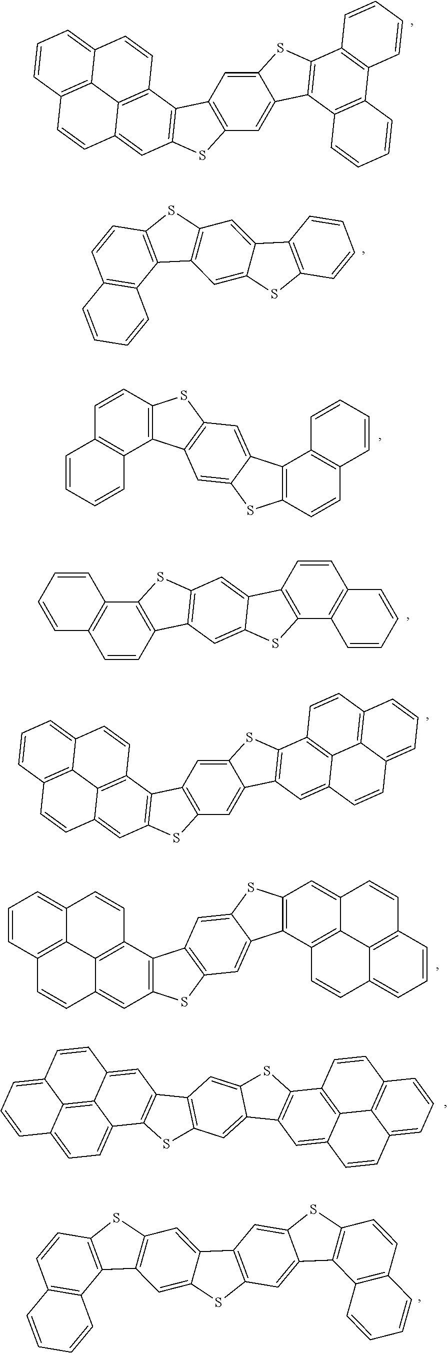

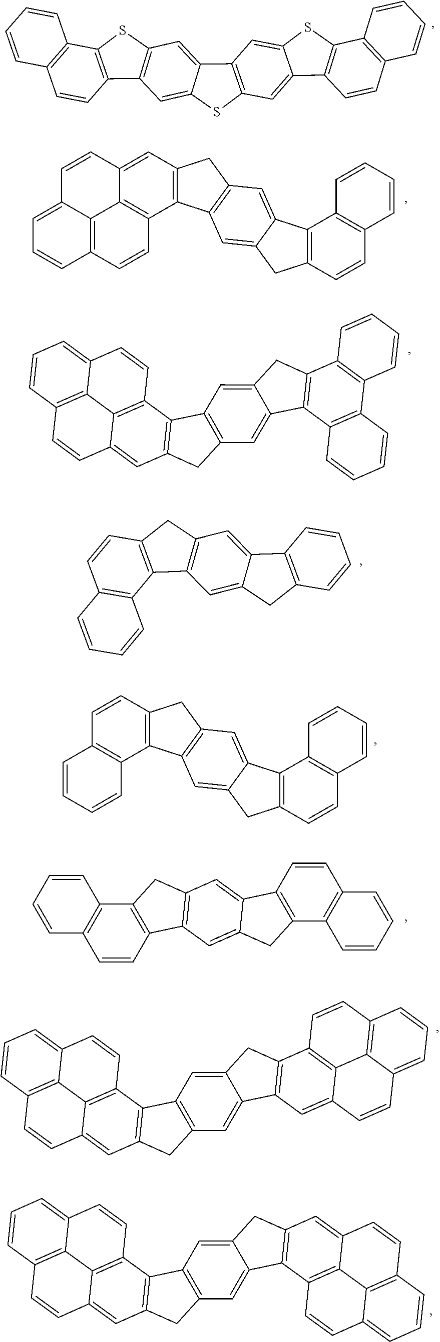

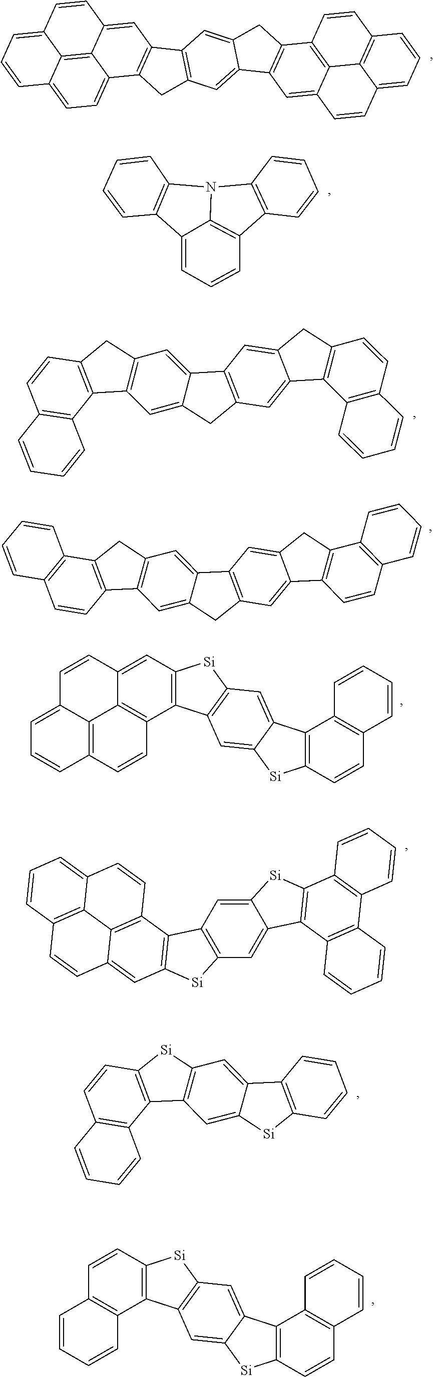

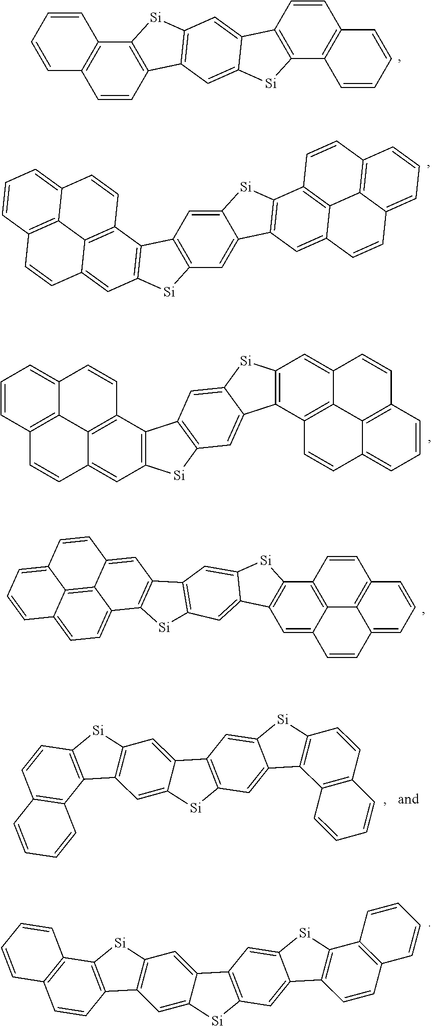

In some embodiments, the acceptor compound comprises at least one moiety selected from the group consisting of:

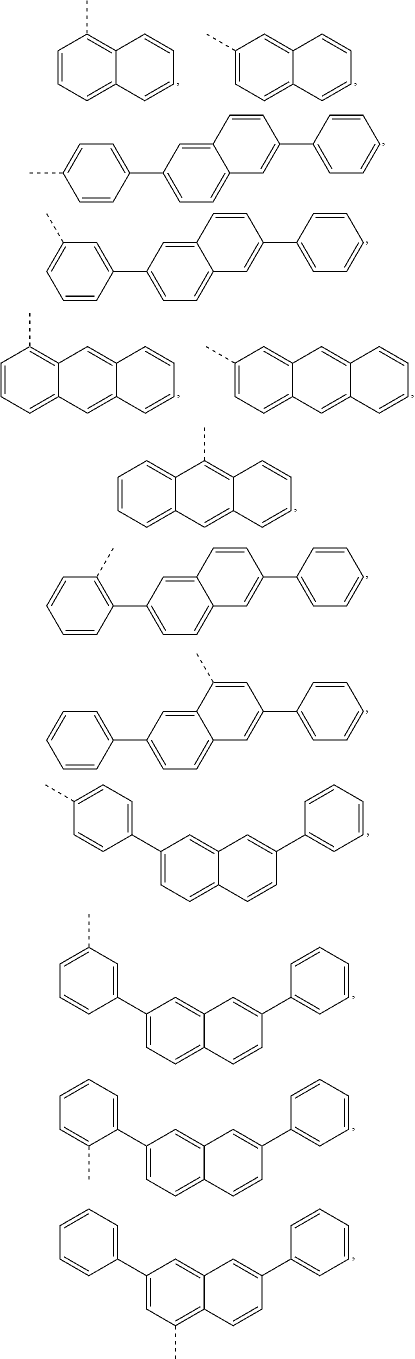

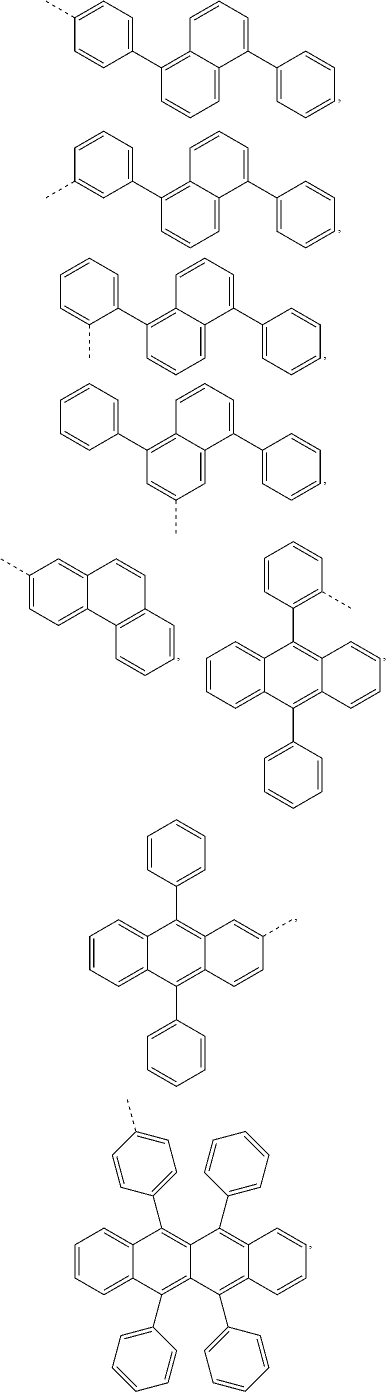

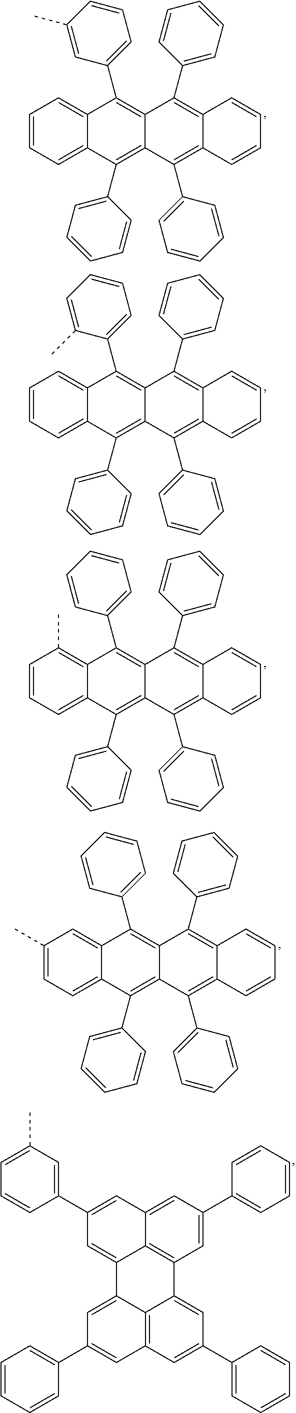

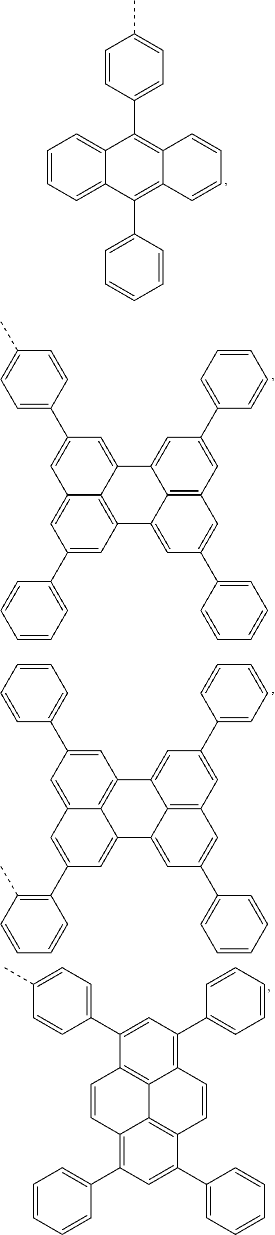

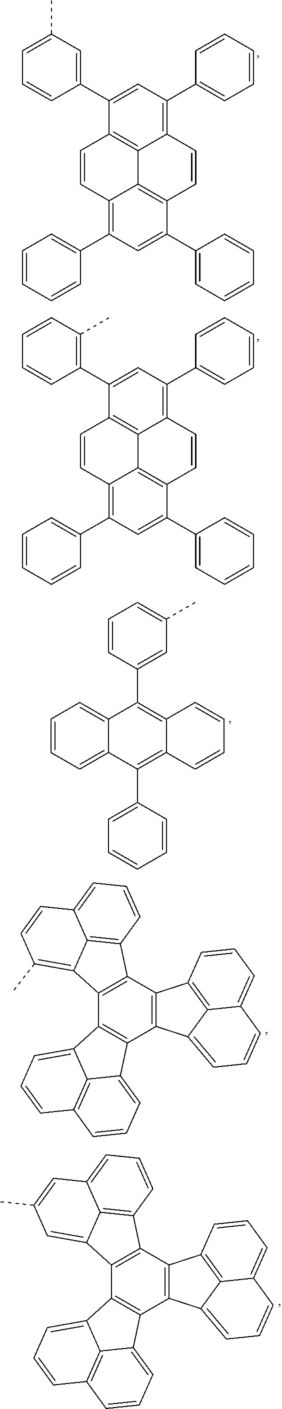

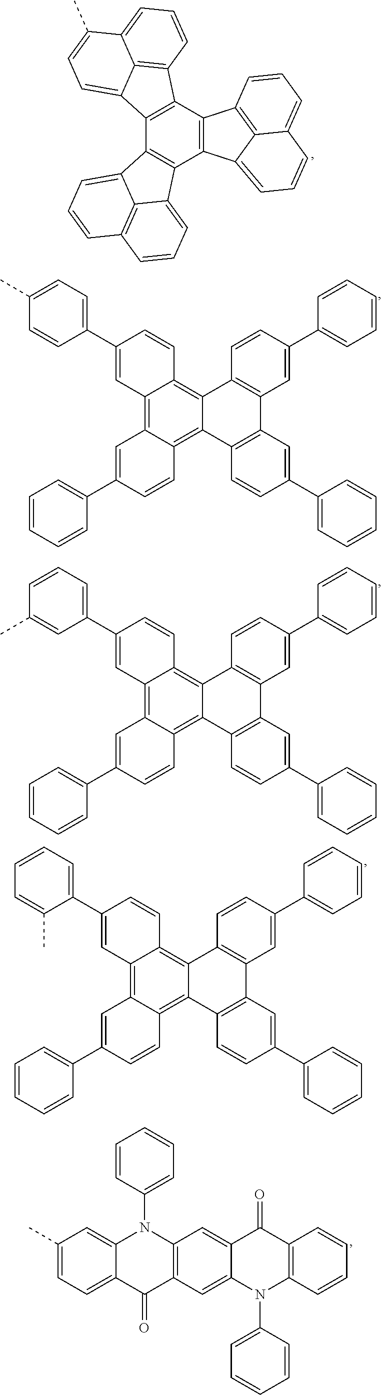

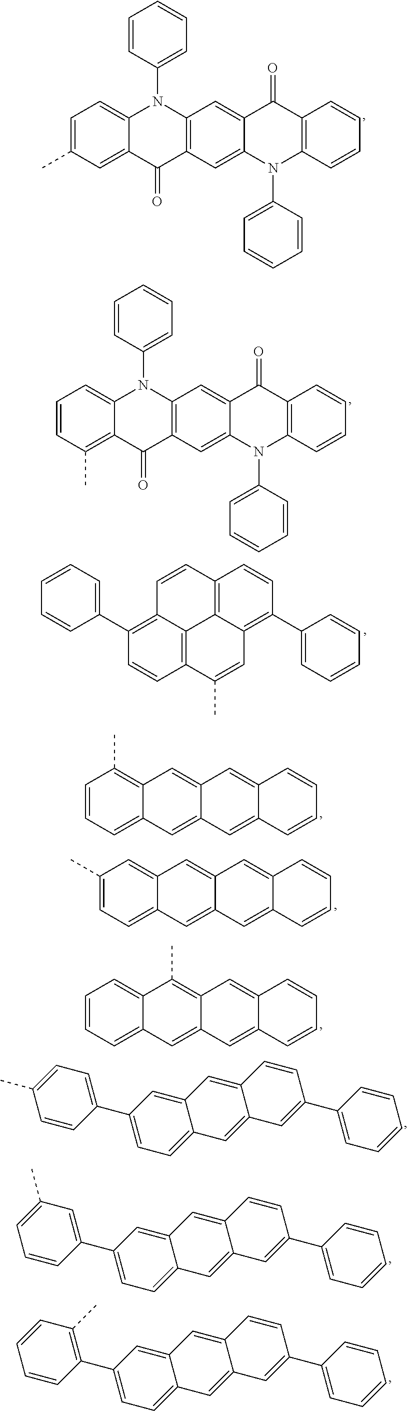

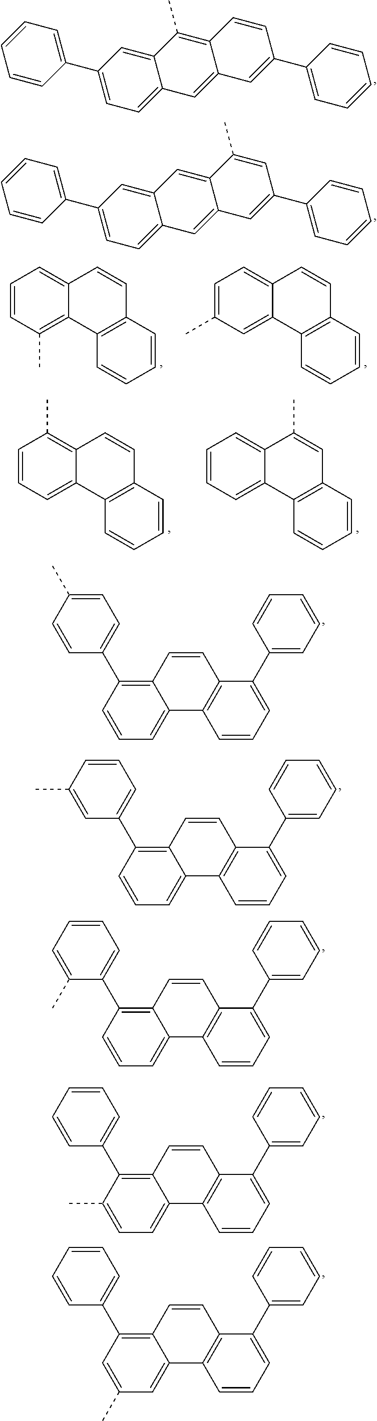

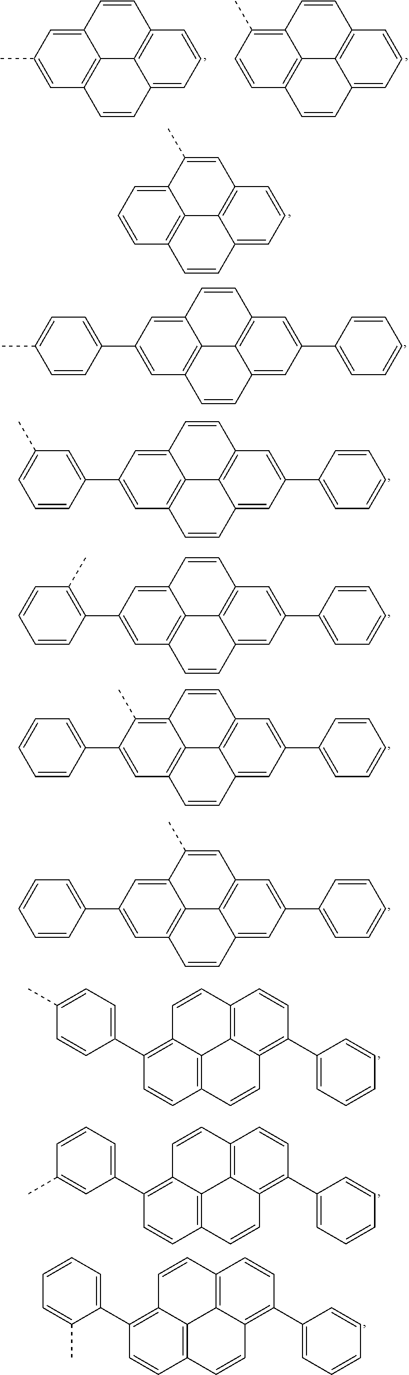

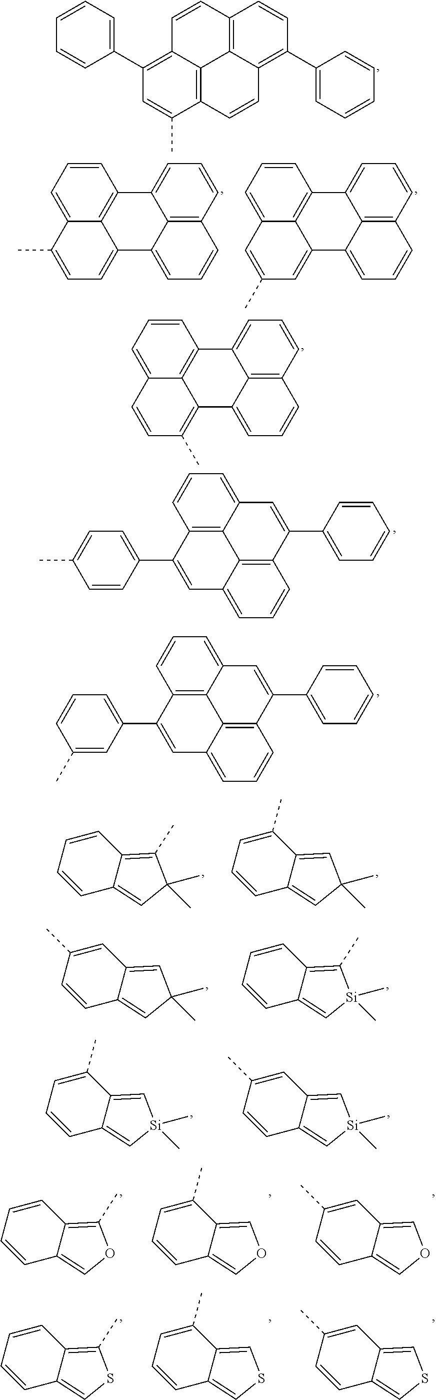

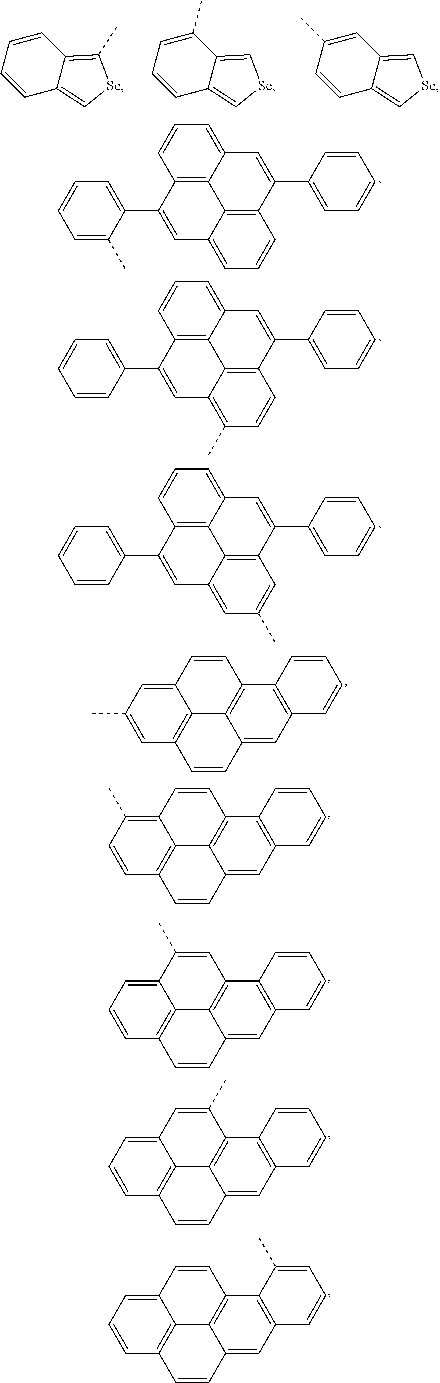

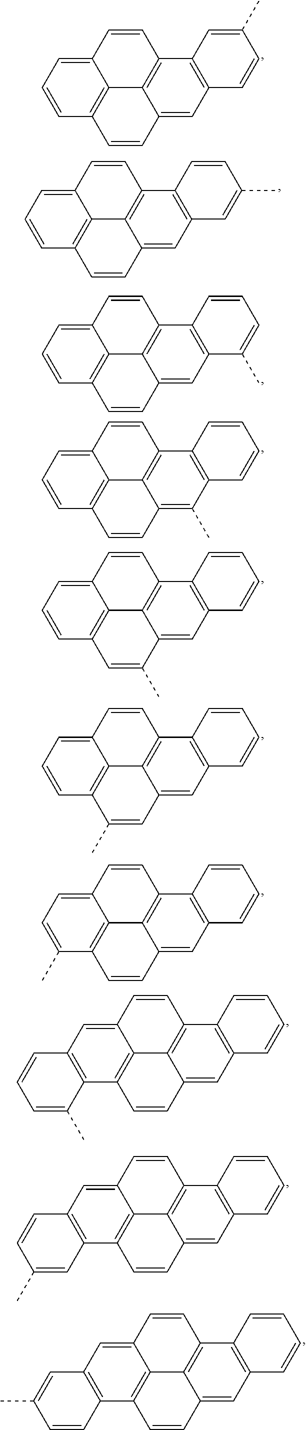

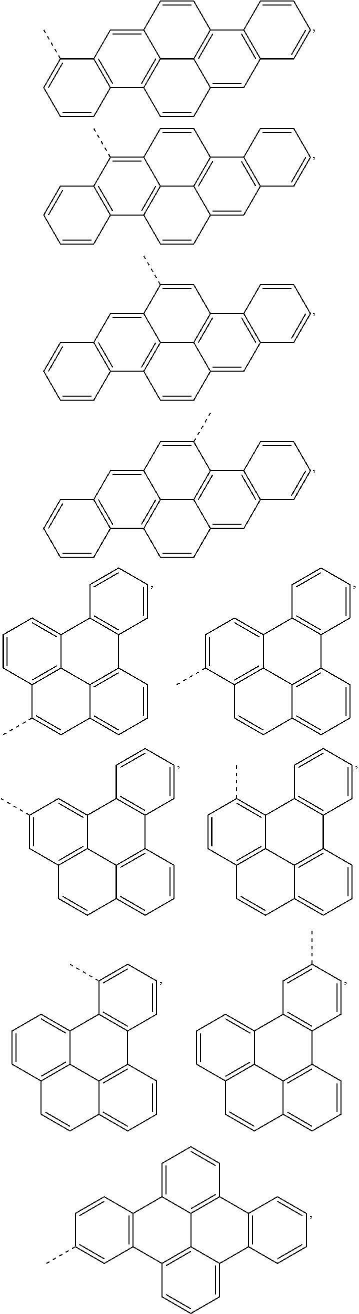

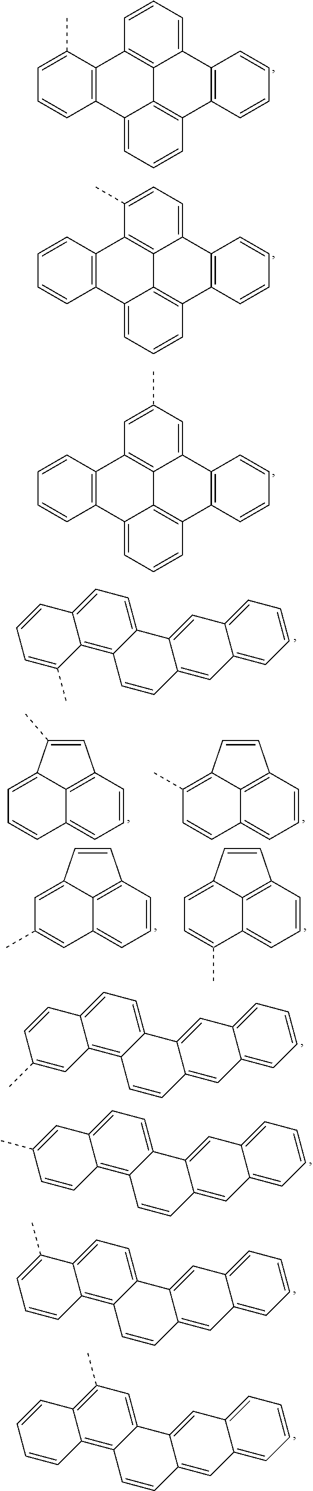

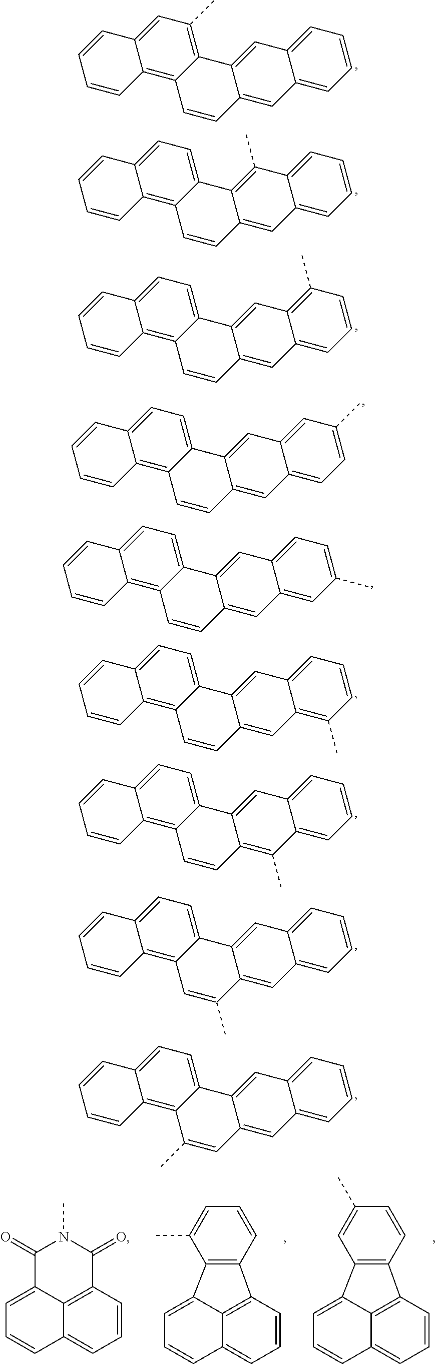

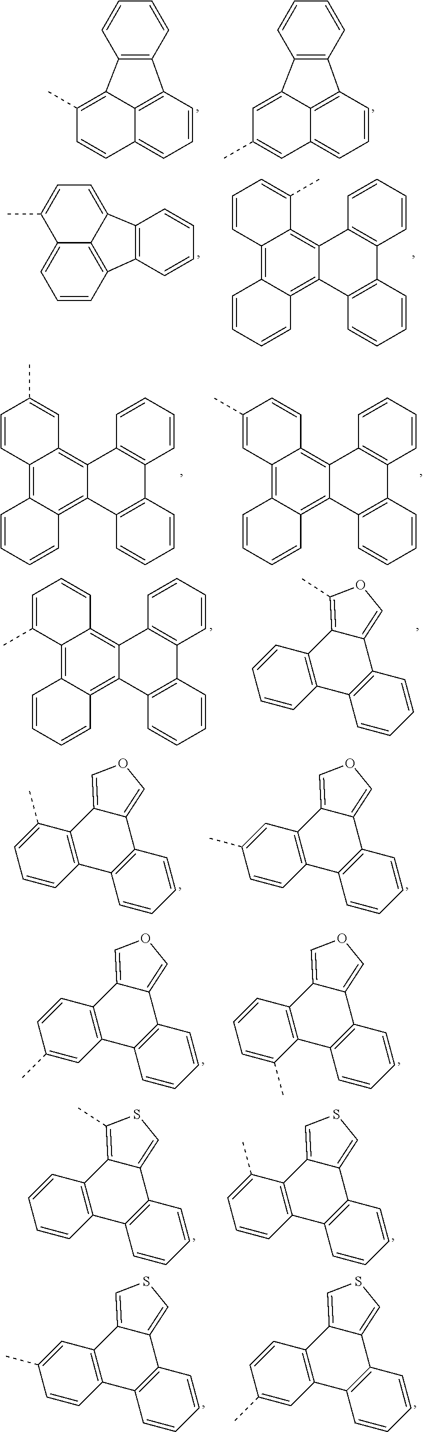

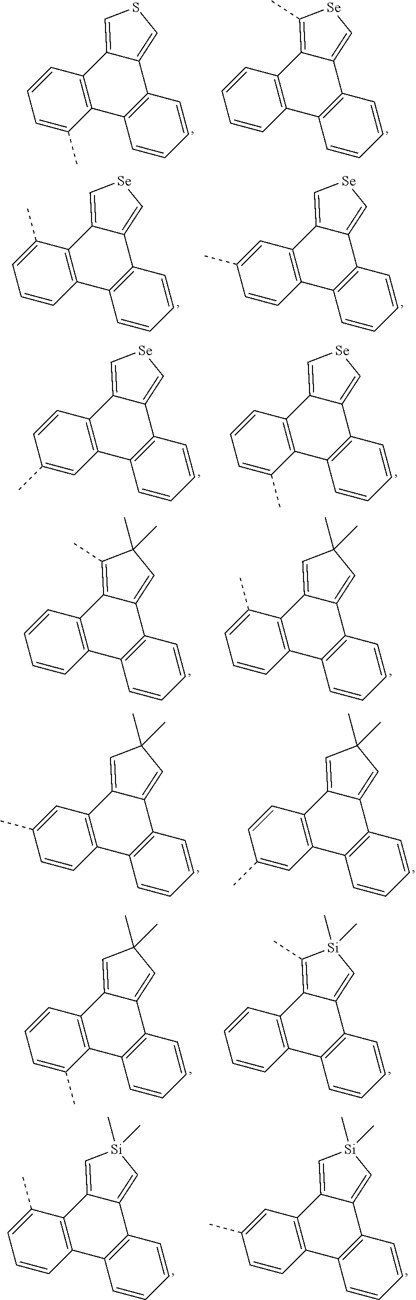

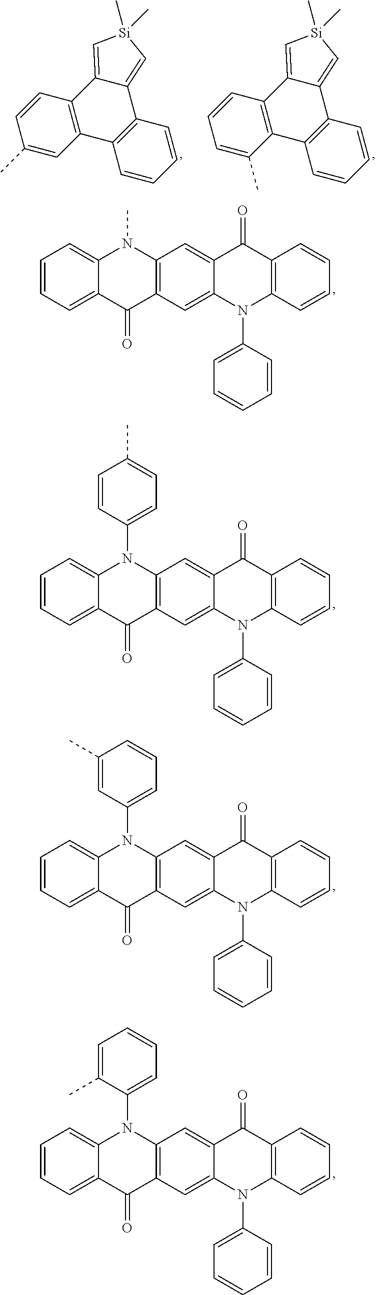

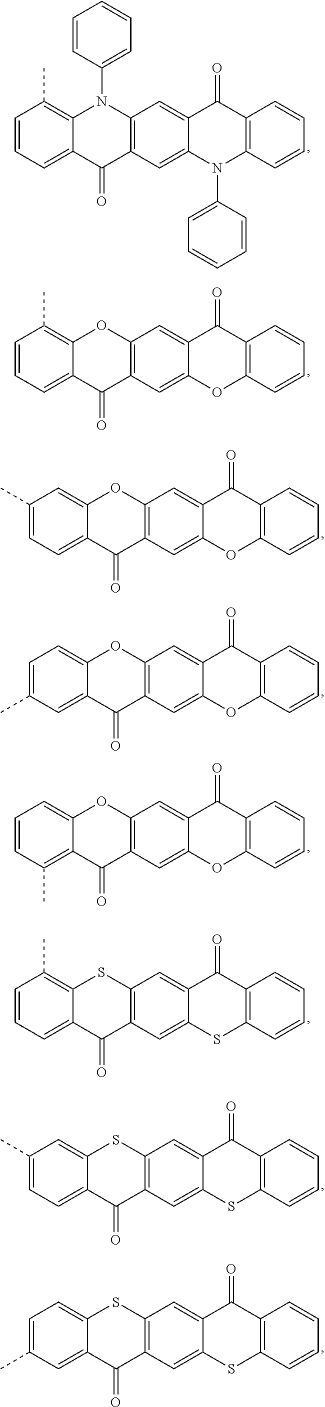

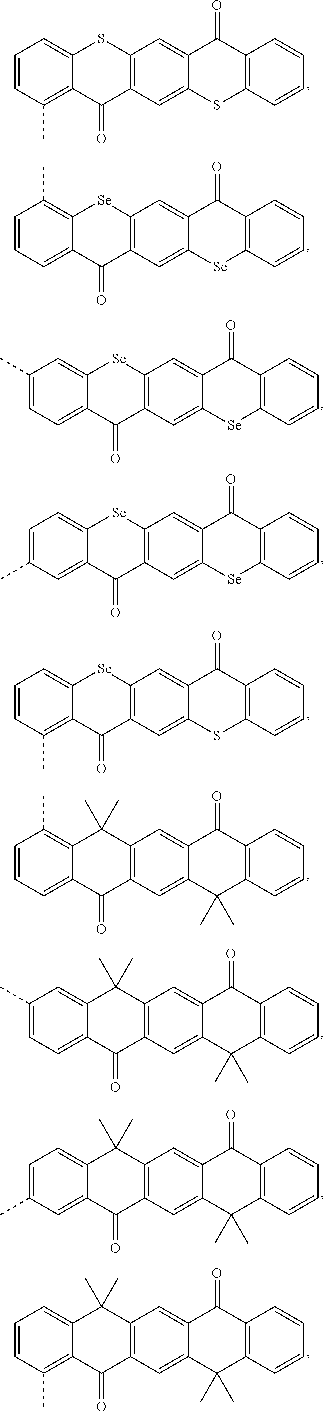

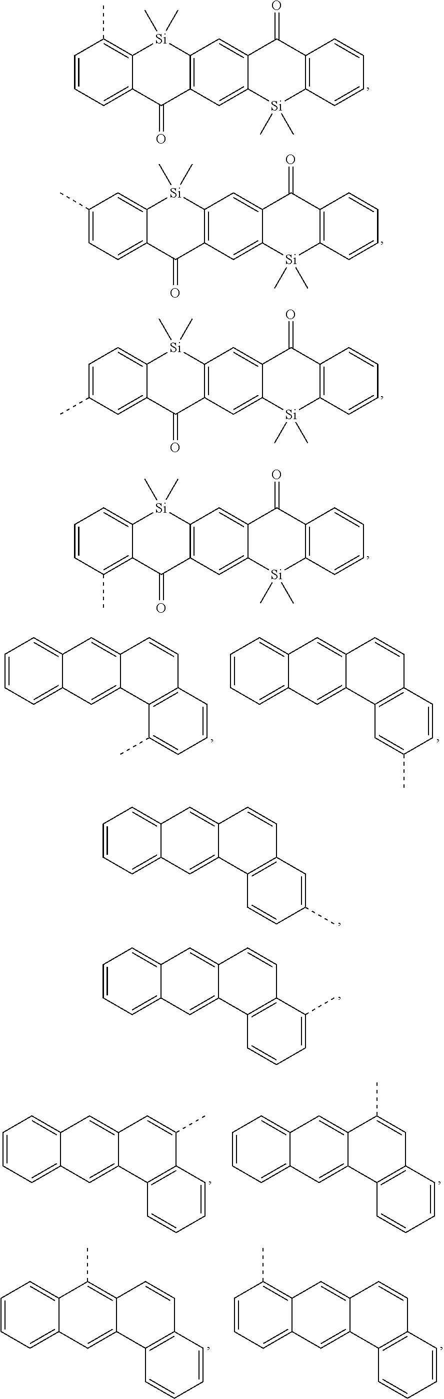

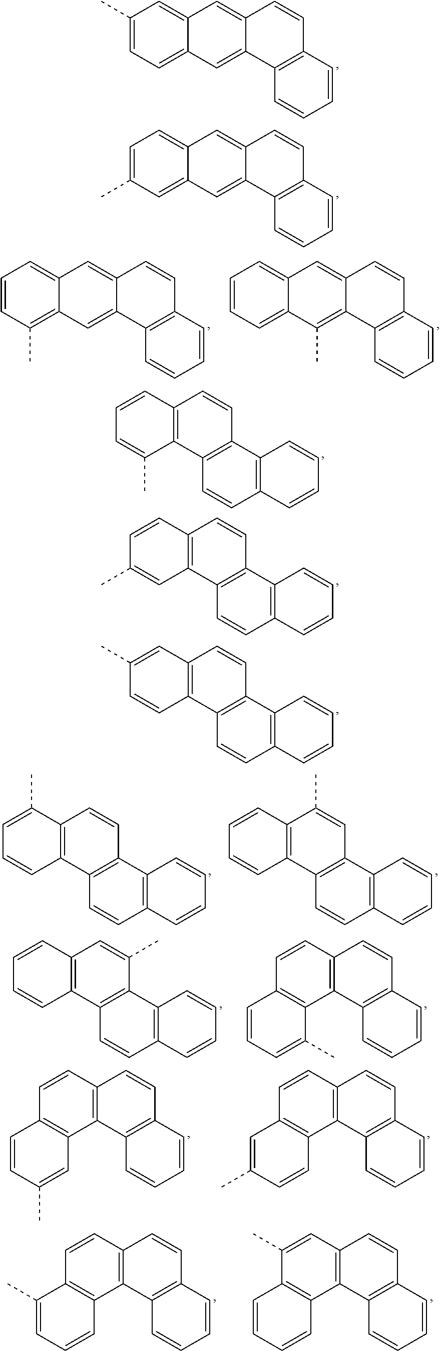

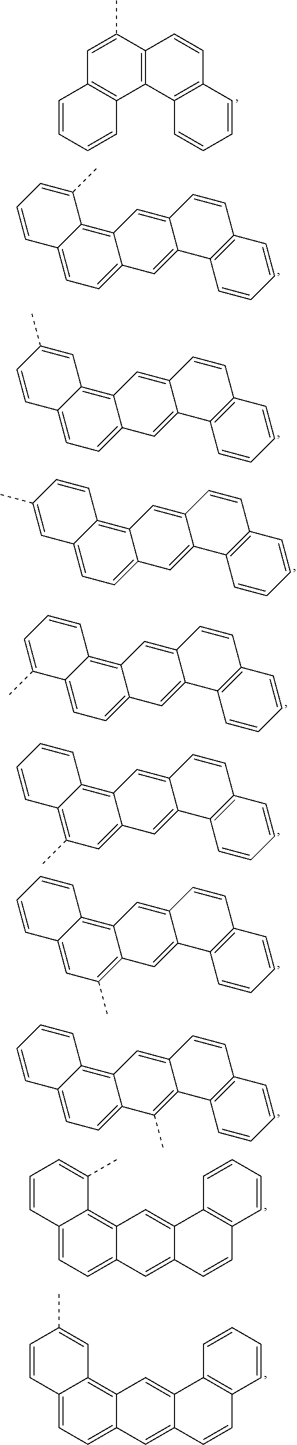

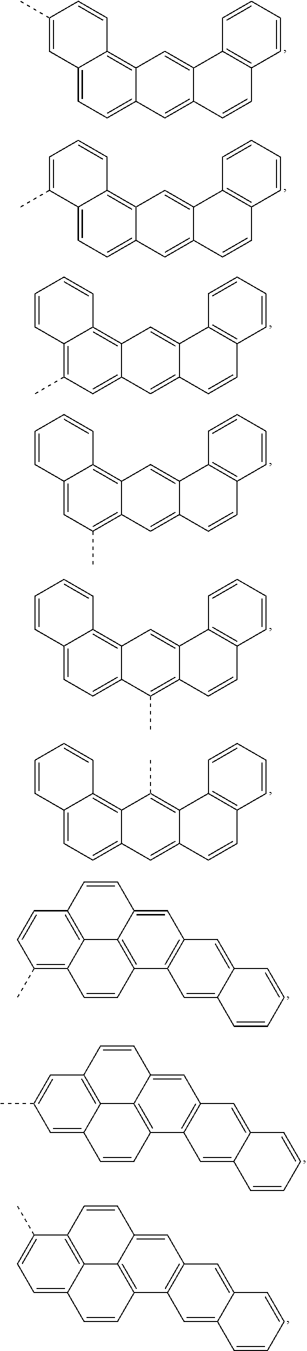

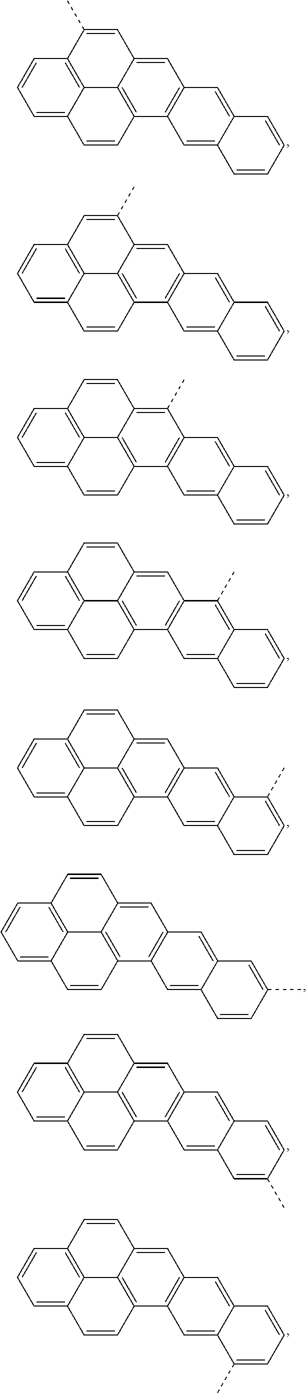

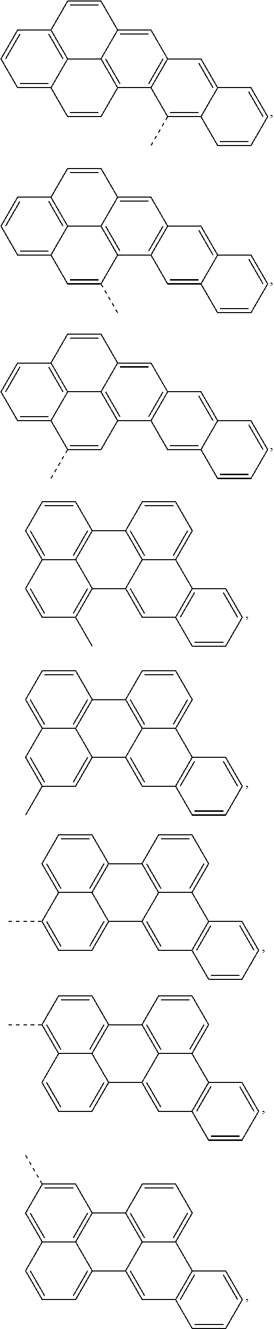

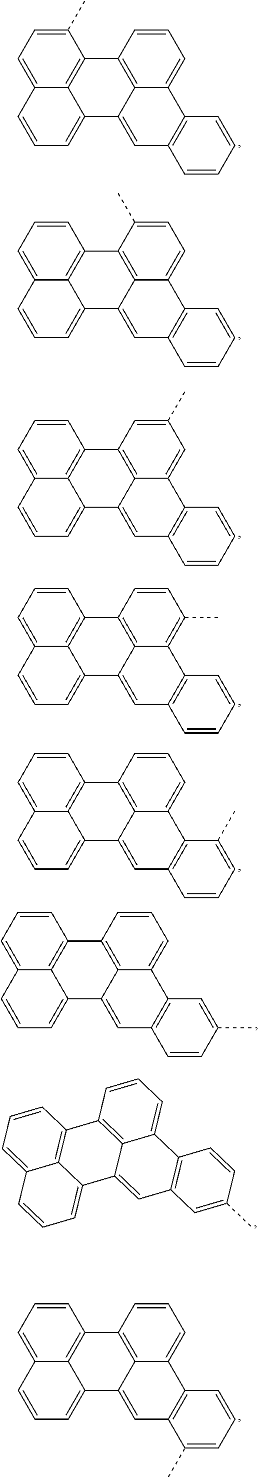

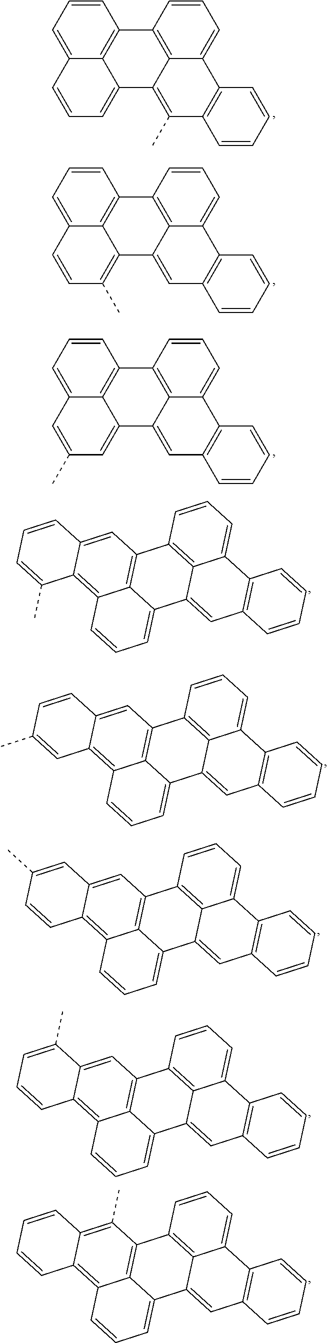

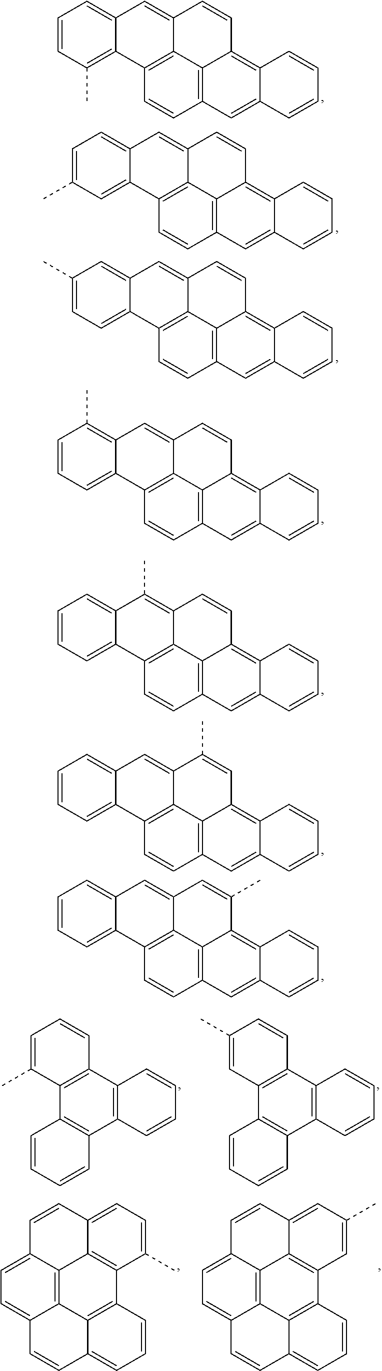

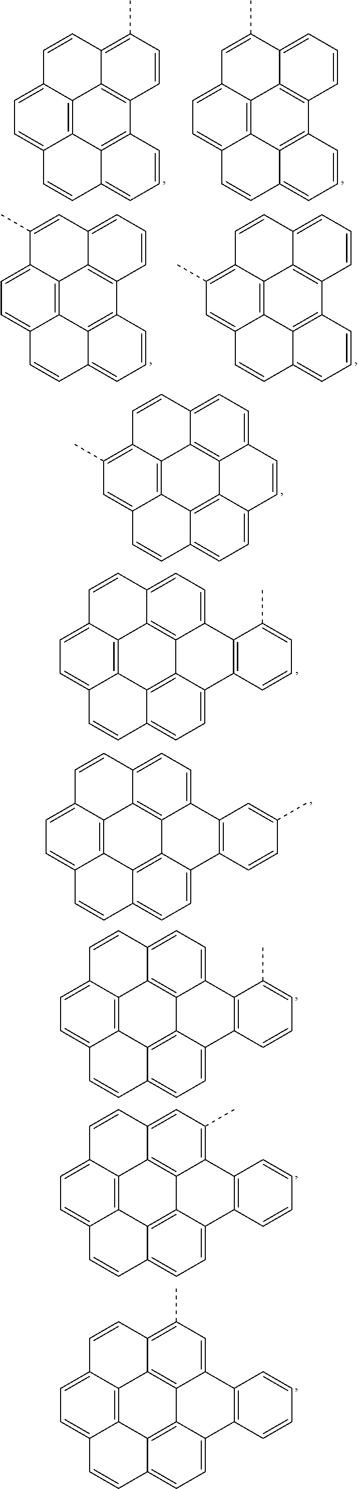

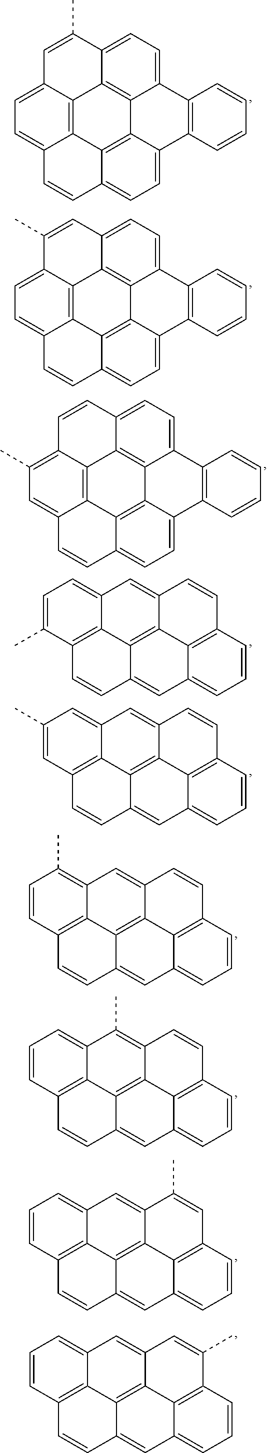

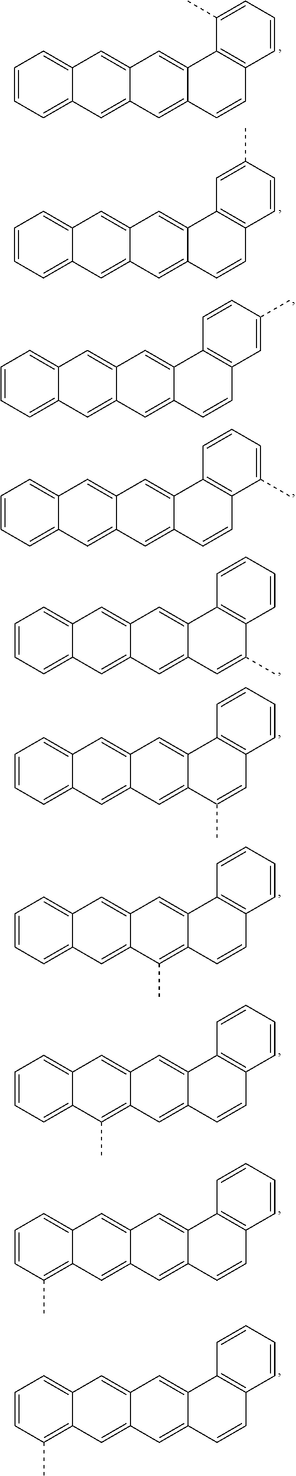

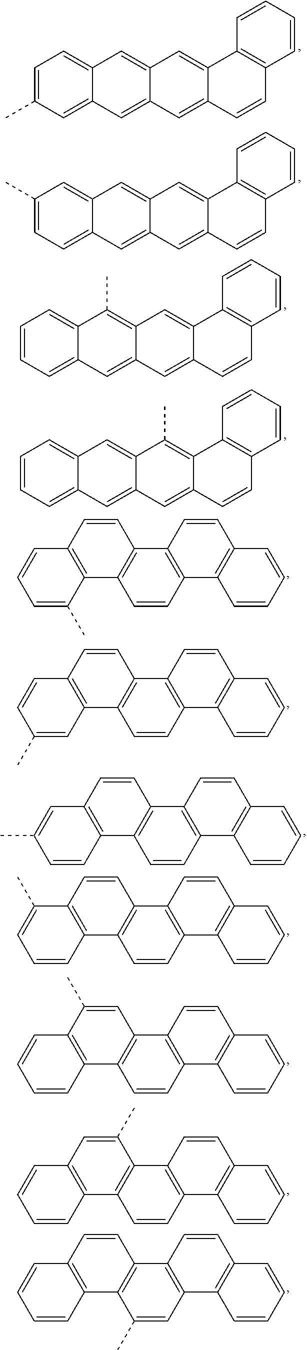

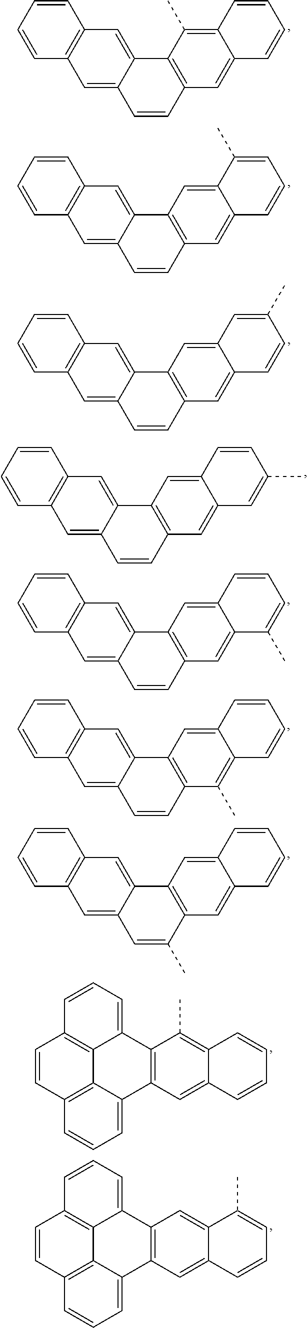

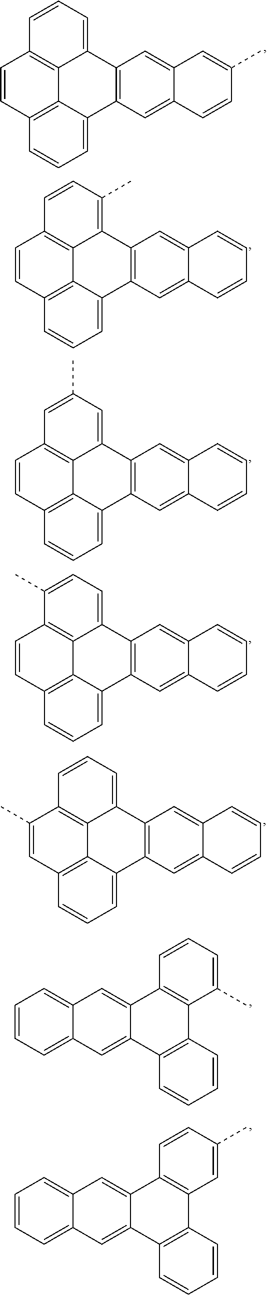

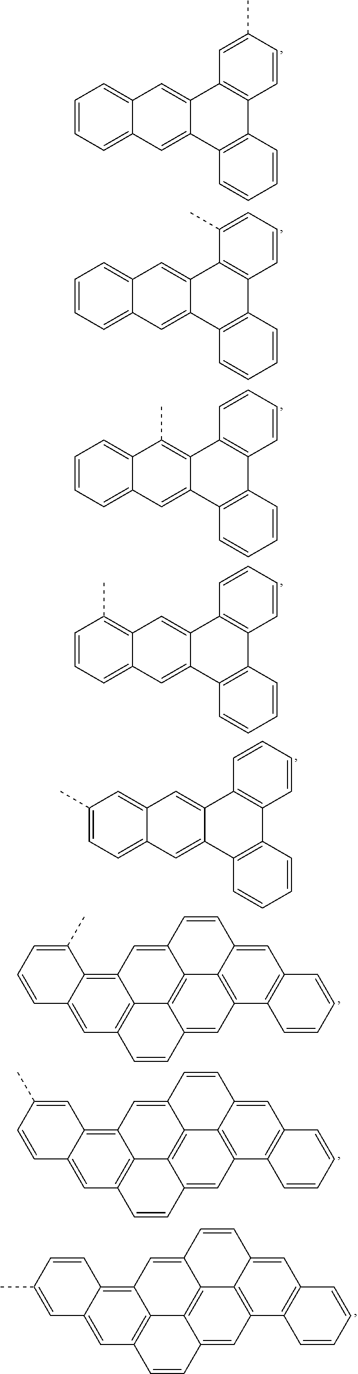

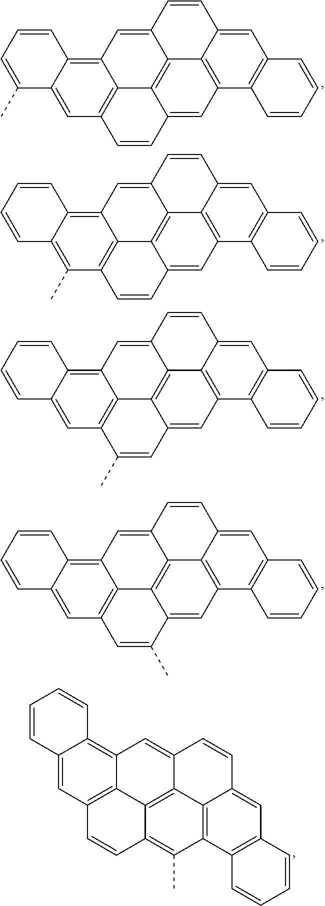

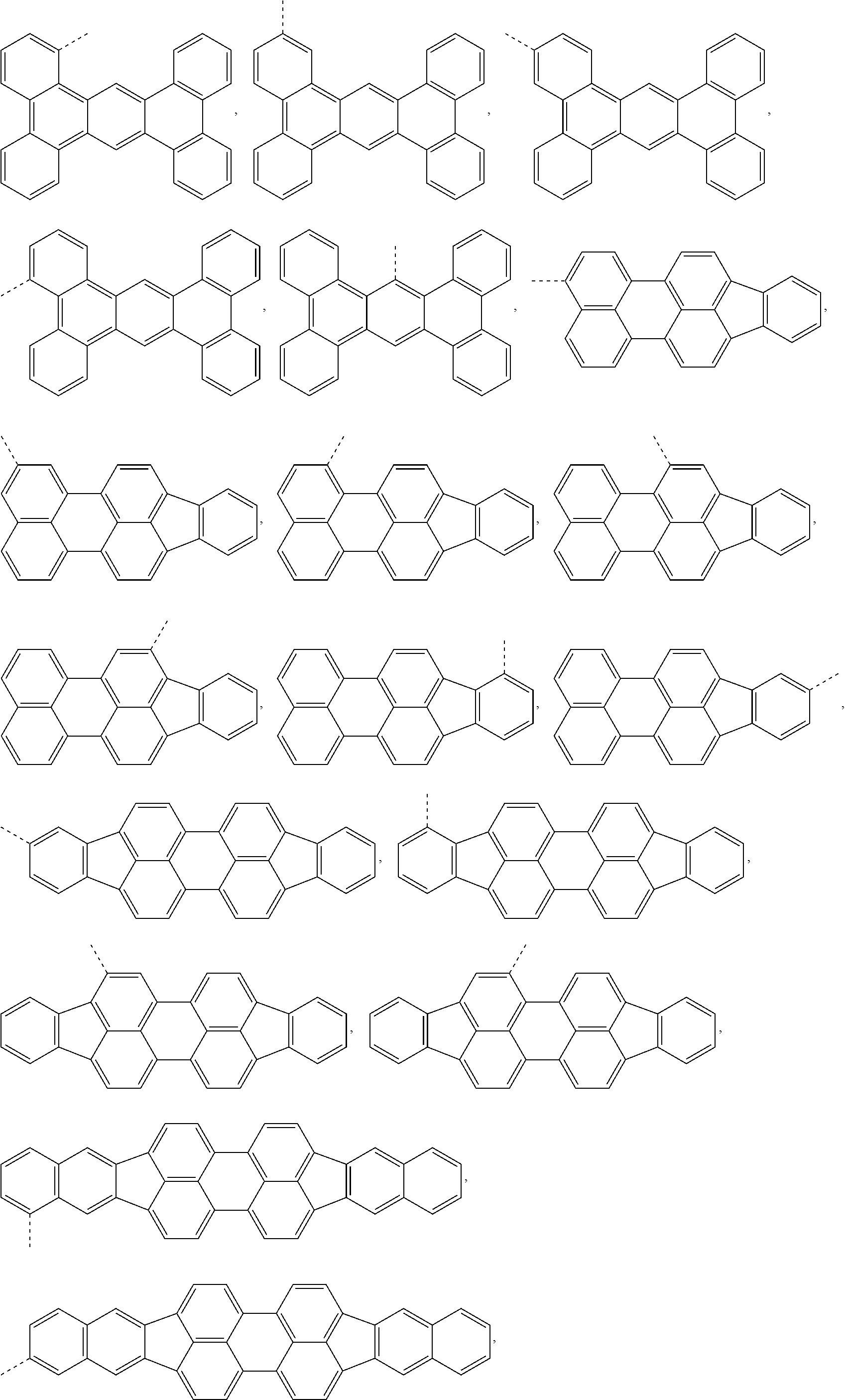

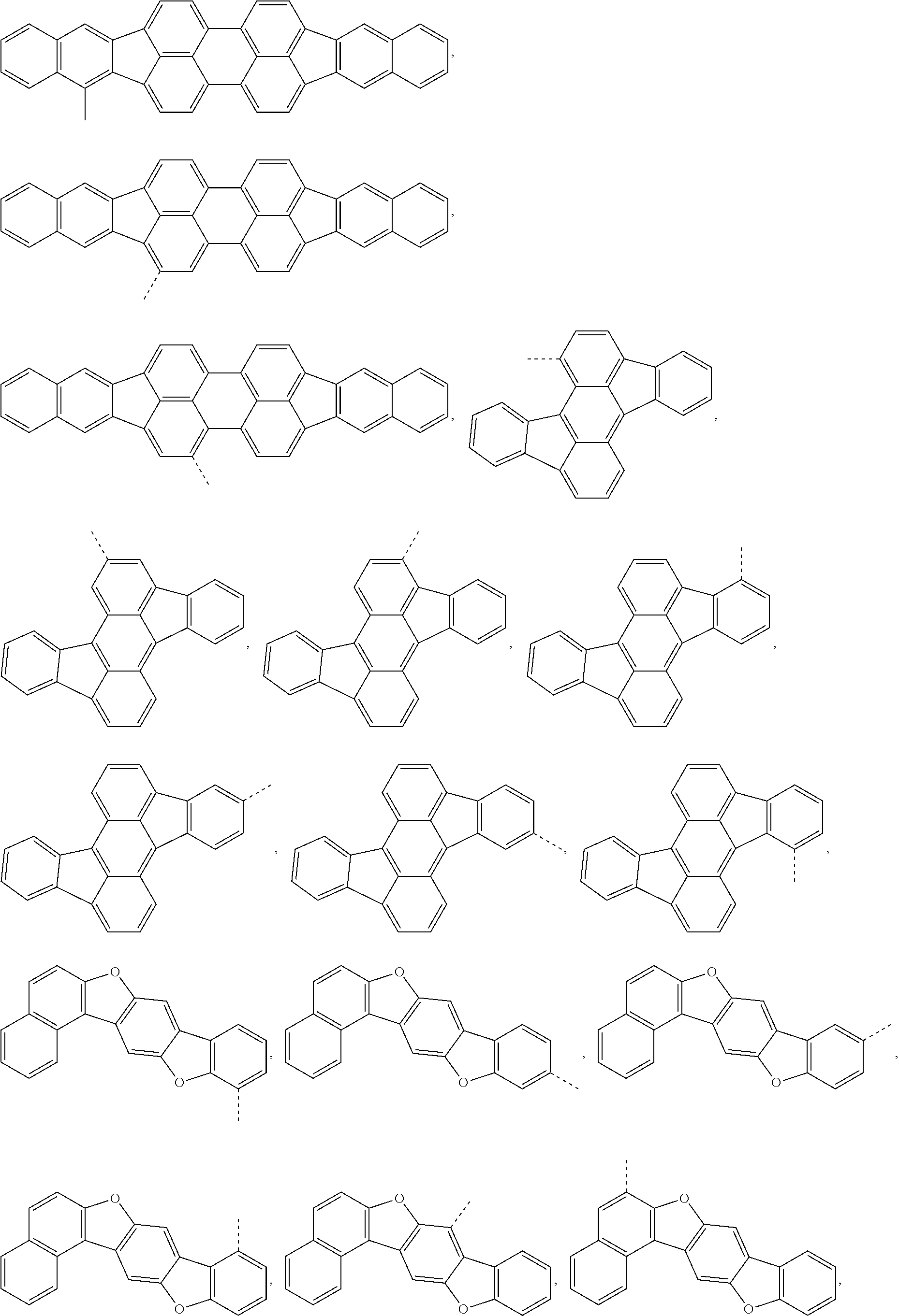

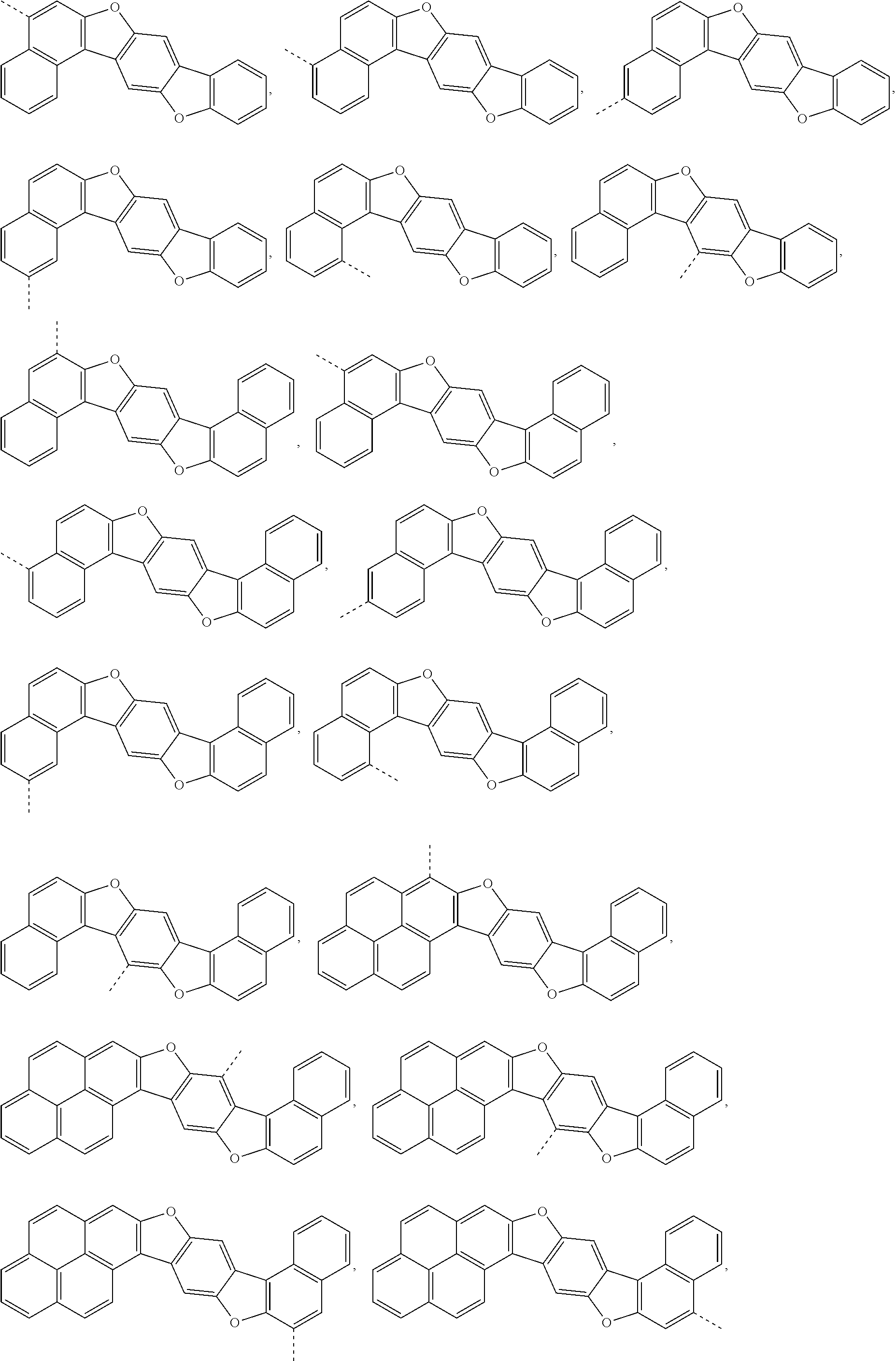

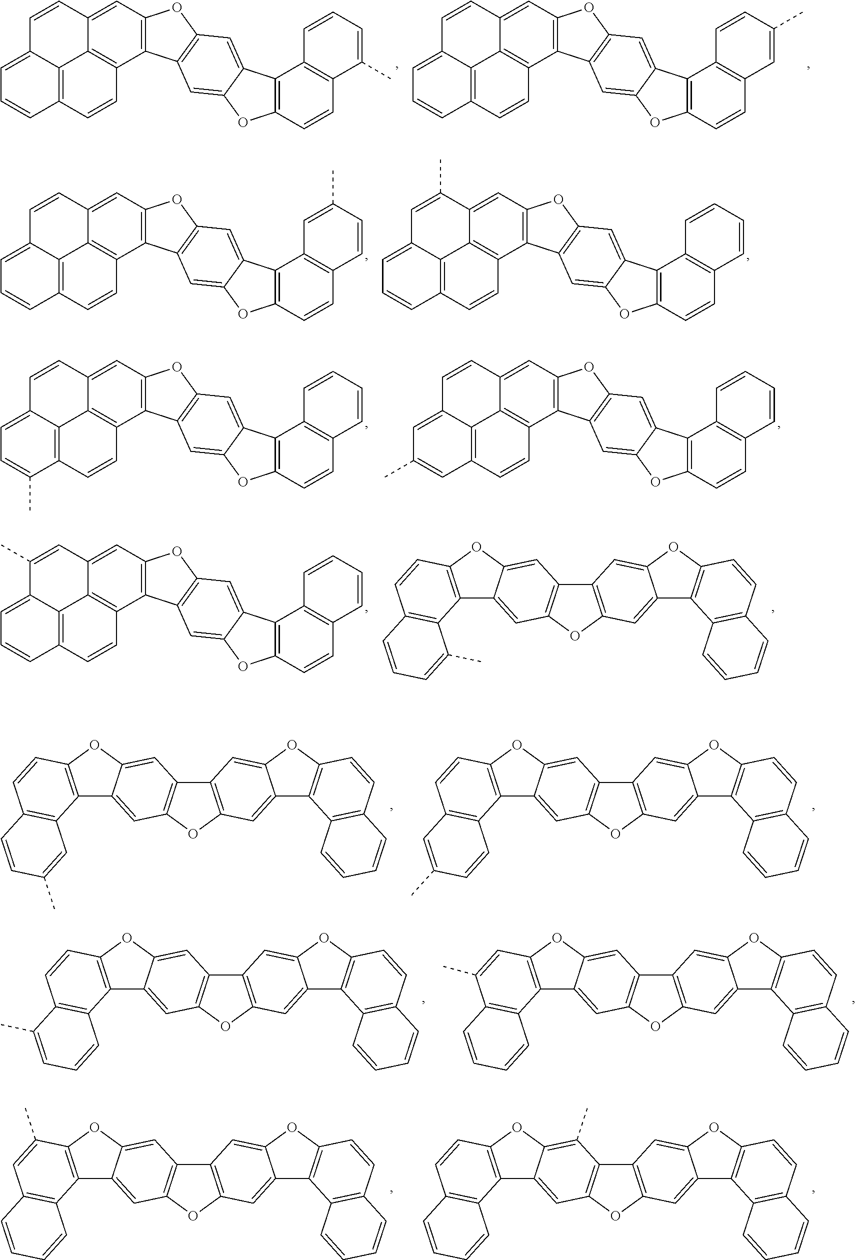

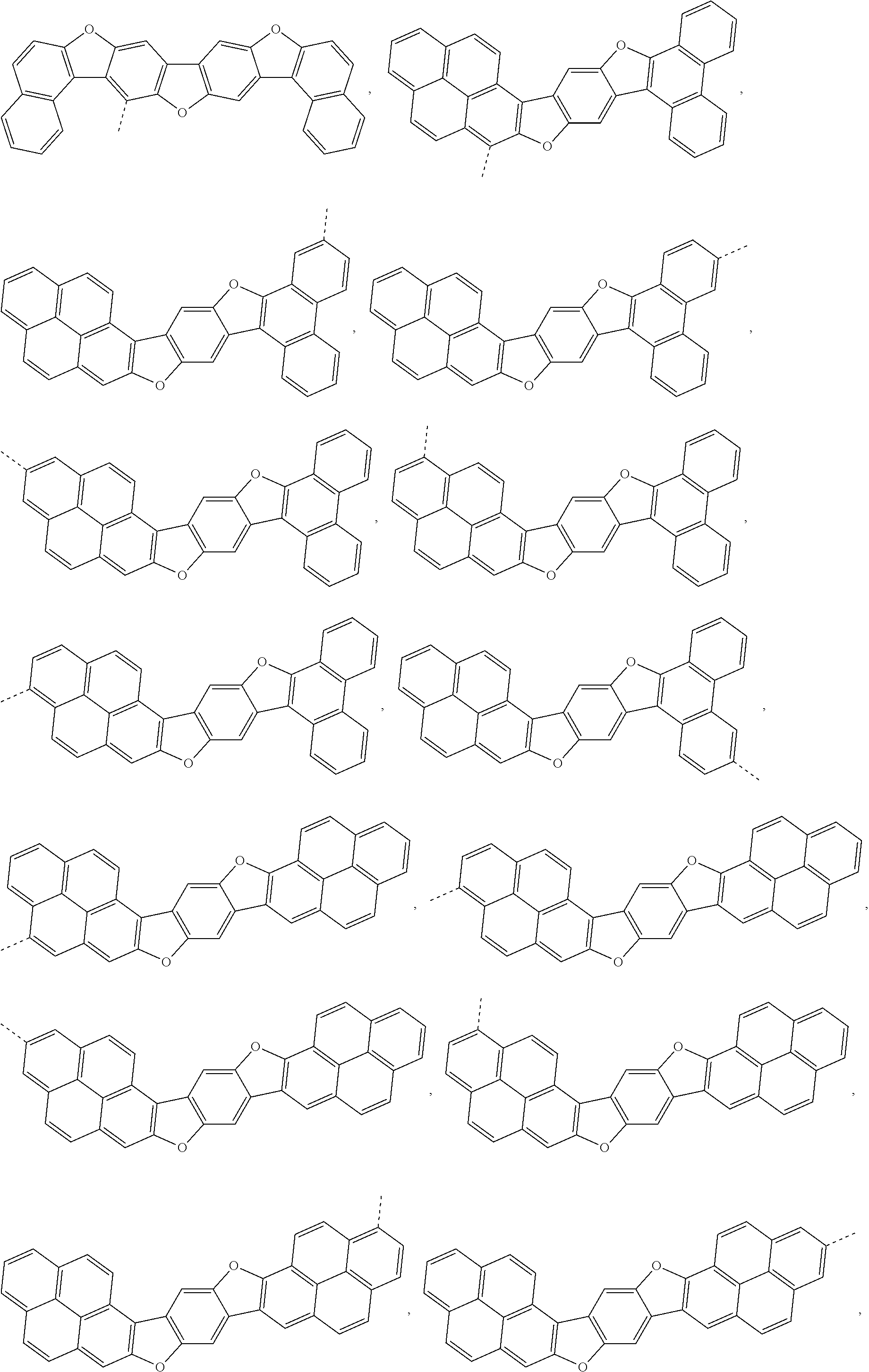

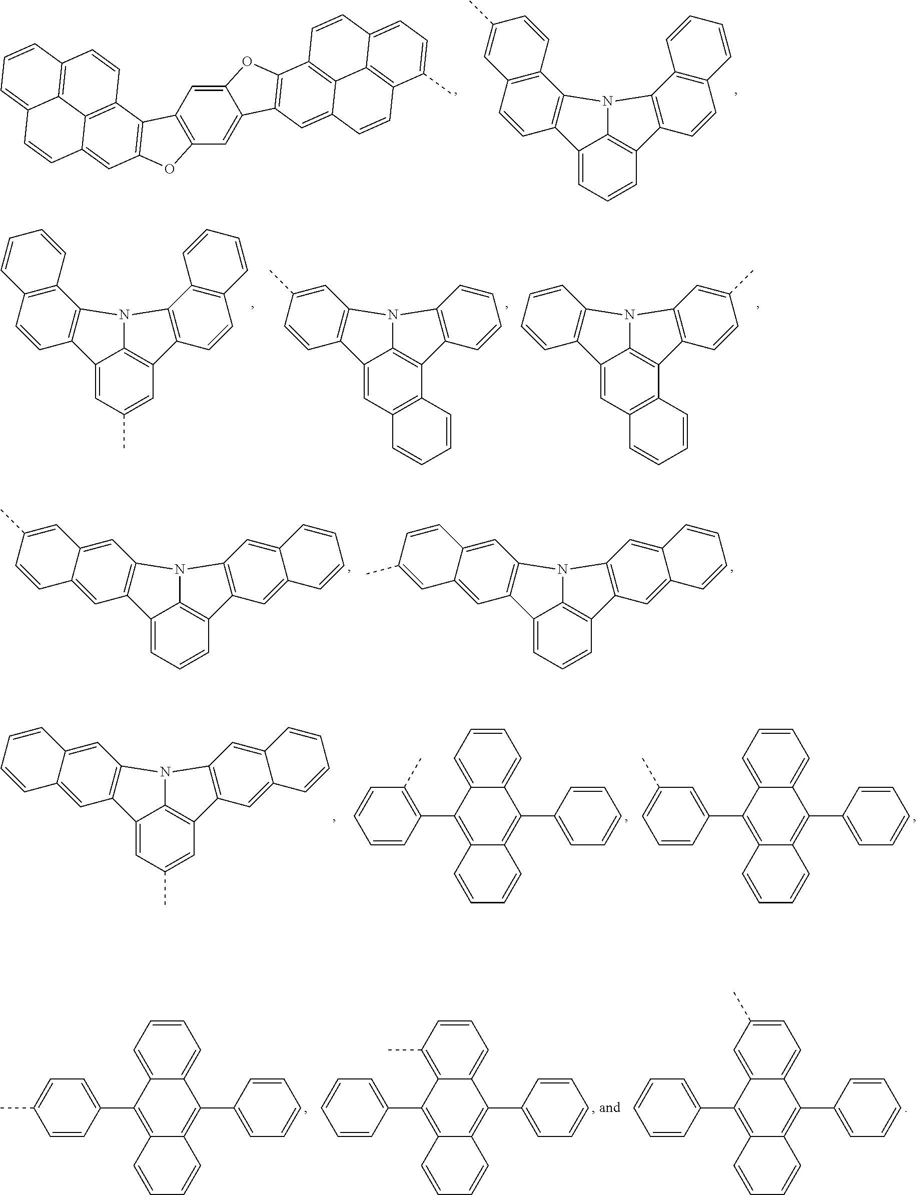

In some embodiments, the acceptor compound is selected from the group consisting of the structures of the following ACCEPTOR LIST:

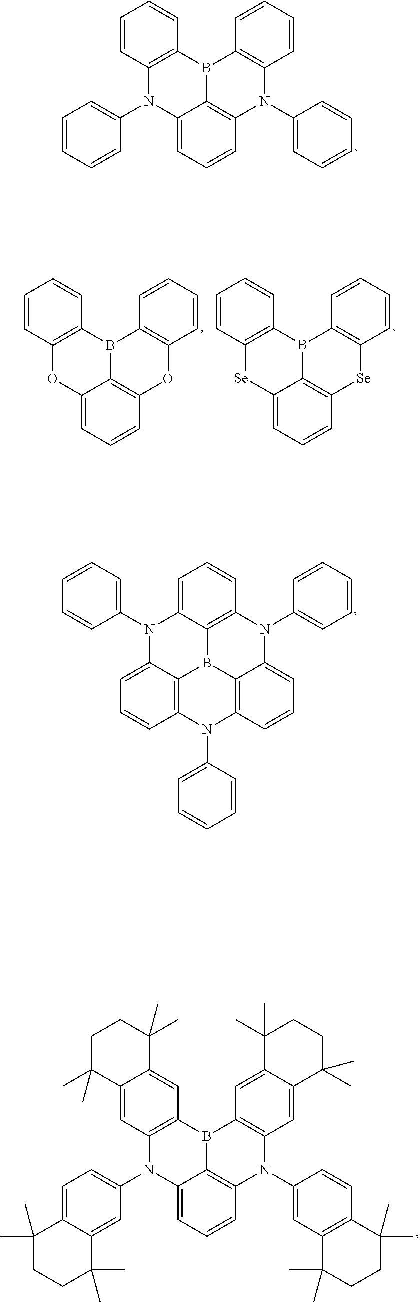

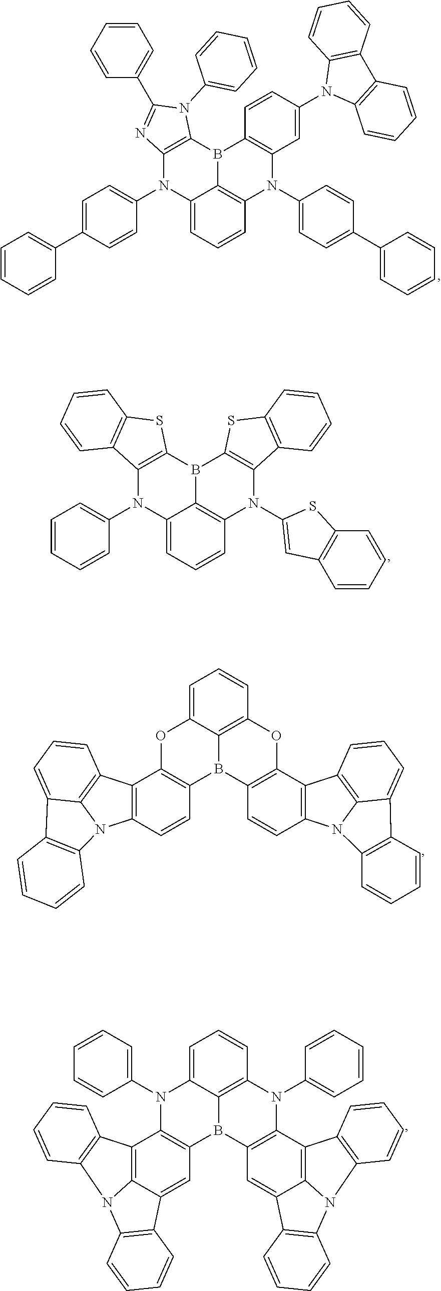 [SMILES: c1ccc(-c2ccc(N3c4cc(-n5c6ccccc6c6ccccc65)ccc4B4c5c3cccc5N(c3ccc(-c5ccccc5)cc3)c3nc(-c5ccccc5)n(-c5ccccc5)c34)cc2)cc1 ; c1ccc(N2c3cccc4c3B(c3sc5ccccc5c32)c2sc3ccccc3c2N4c2cc3ccccc3s2)cc1 ; c1cc2c3c(c1)Oc1c(ccc4c1c1cccc5c6ccccc6n4c51)B3c1ccc3c(c1O2)c1cccc2c4ccccc4n3c21 ; c1ccc(N2c3cccc4c3B(c3cc5c6ccccc6n6c7ccccc7c(c32)c56)c2cc3c5ccccc5n5c6ccccc6c(c2N4c2ccccc2)c35)cc1]

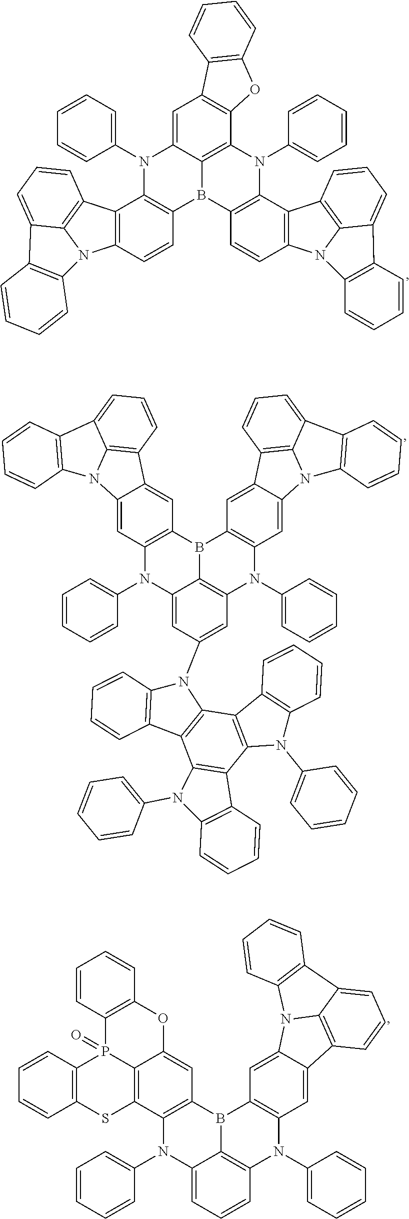 [SMILES: c1ccc(N2c3cc4c(oc5ccccc54)c4c3B(c3ccc5c(c32)c2cccc3c6ccccc6n5c32)c2ccc3c(c2N4c2ccccc2)c2cccc4c5ccccc5n3c42)cc1 ; c1ccc(N2c3cc4c(cc3B3c5cc6c7cccc8c9ccccc9n(c6cc5N(c5ccccc5)c5cc(-n6c9ccccc9c9c%10c(c%11ccccc%11n%10-c%10ccccc%10)c%10c(c%11ccccc%11n%10-c%10ccccc%10)c96)cc2c53)c87)c2cccc3c5ccccc5n4c32)cc1 ; O=P12c3ccccc3Oc3cc4c(c(c31)Sc1ccccc12)N(c1ccccc1)c1cccc2c1B4c1cc3c(cc1N2c1ccccc1)c1cccc2c4ccccc4n3c21]

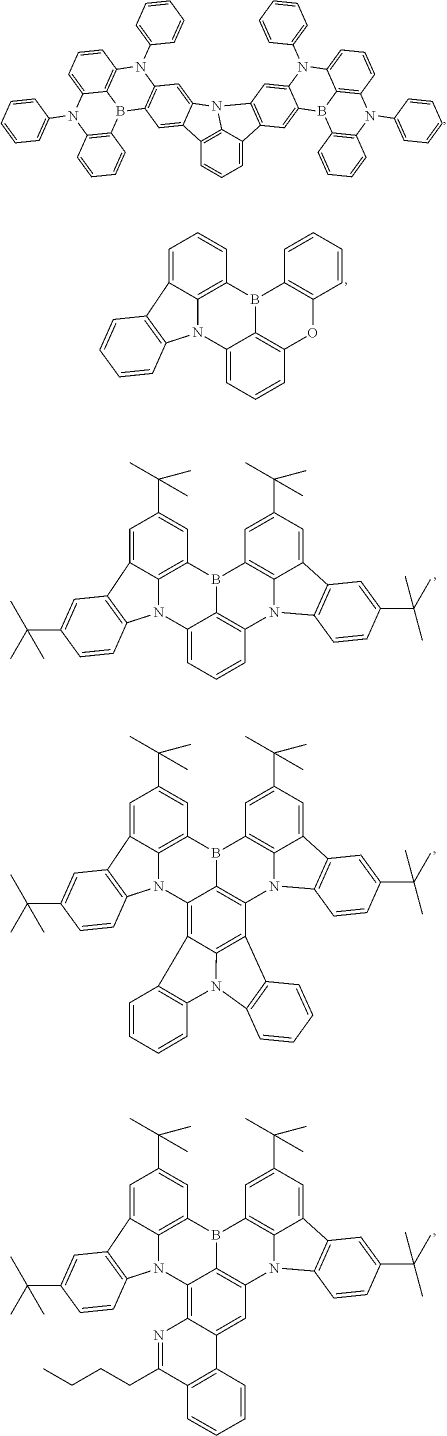

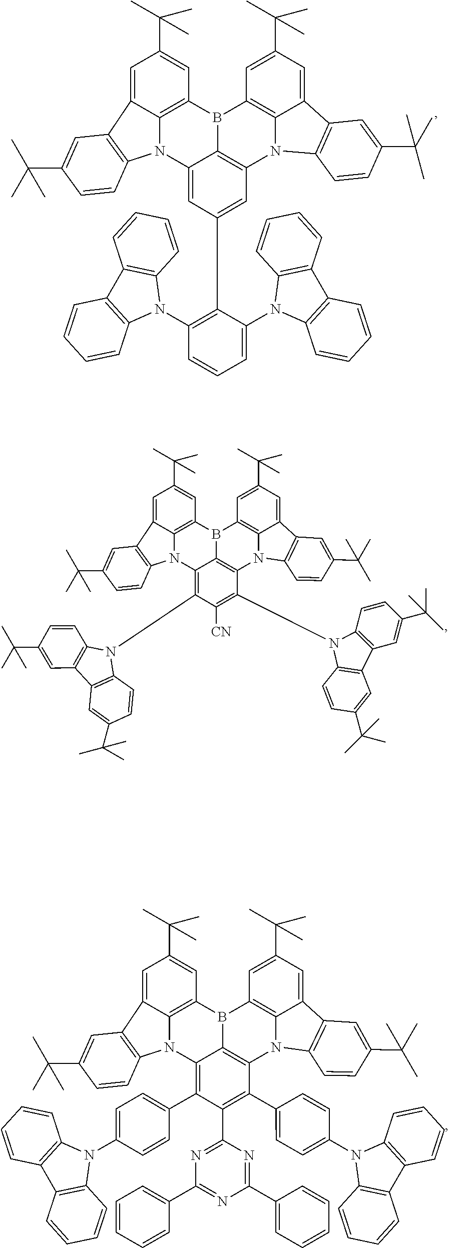

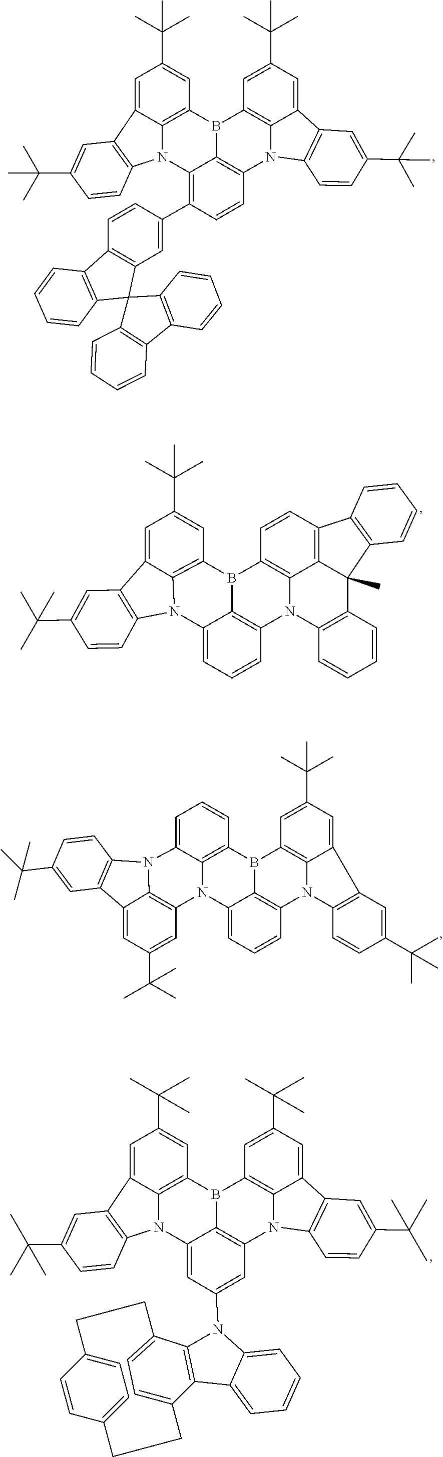

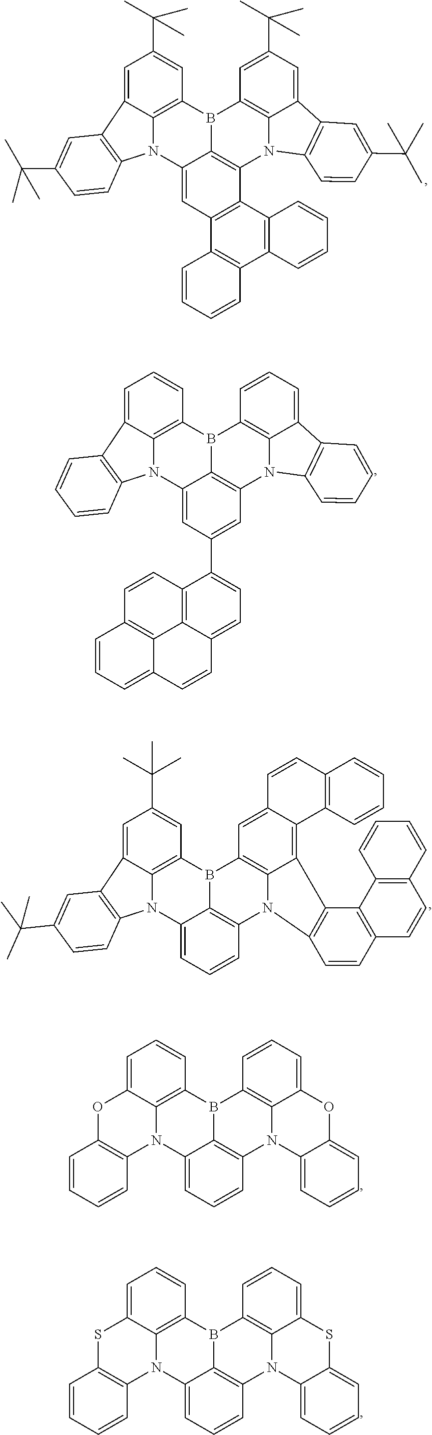

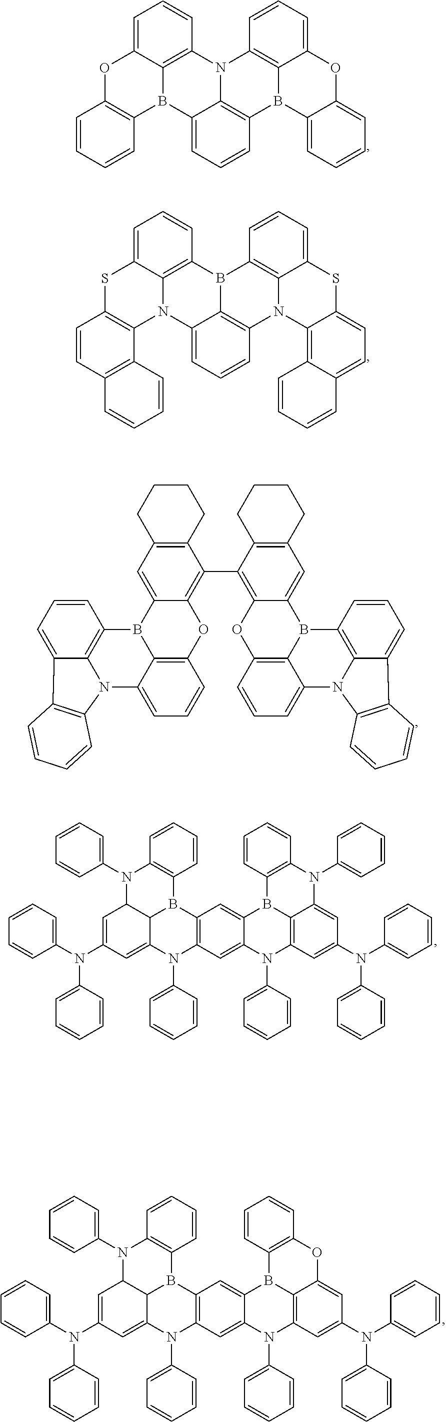

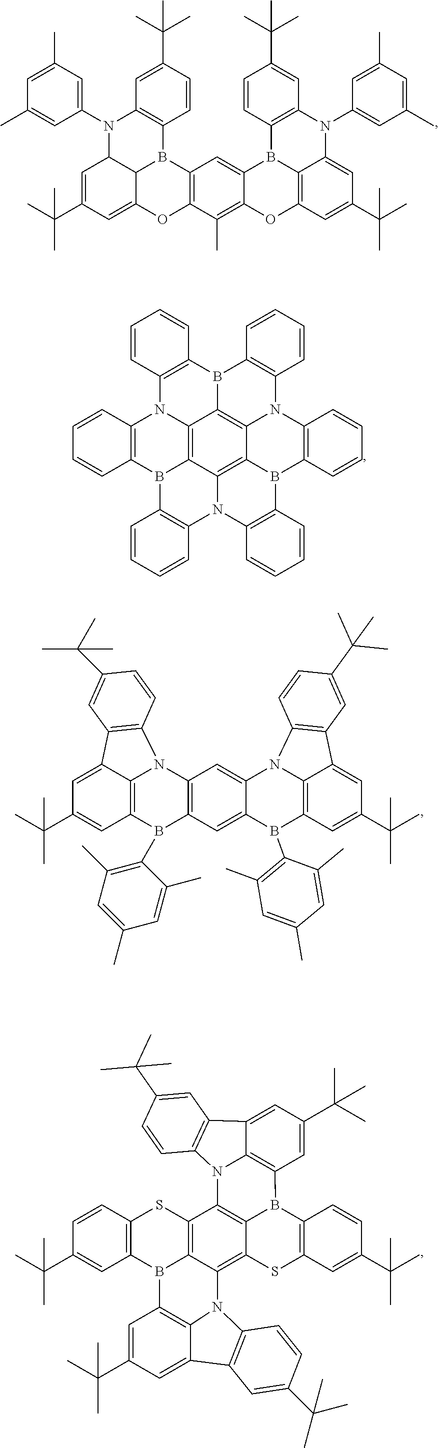

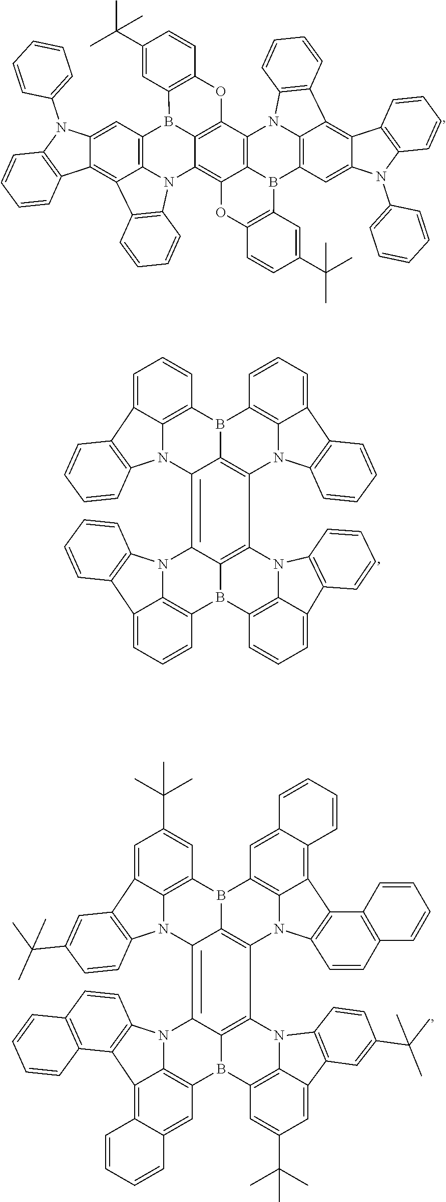 [SMILES: CC(C)(C)c1ccc2c(c1)B1c3c(c4c5c(c3-n3c6ccccc6c6c7c8ccccc8n(-c8ccccc8)c7cc1c63)Oc1ccc(C(C)(C)C)cc1B5c1cc3c(c5ccccc5n3-c3ccccc3)c3c5ccccc5n-4c13)O2 ; c1ccc2c(c1)c1cccc3c1n2-c1c2c(c4c5c1-n1c6ccccc6c6cccc(c61)B5c1cccc5c6ccccc6n-4c15)-n1c4ccccc4c4cccc(c41)B23 ; CC(C)(C)c1ccc2c(c1)c1cc(C(C)(C)C)cc3c1n2-c1c2c(c4c5c1-n1c6ccc7ccccc7c6c6c7ccccc7cc(c61)B5c1cc(C(C)(C)C)cc5c6cc(C(C)(C)C)ccc6n-4c15)-n1c4ccc5ccccc5c4c4c5ccccc5cc(c41)B23]

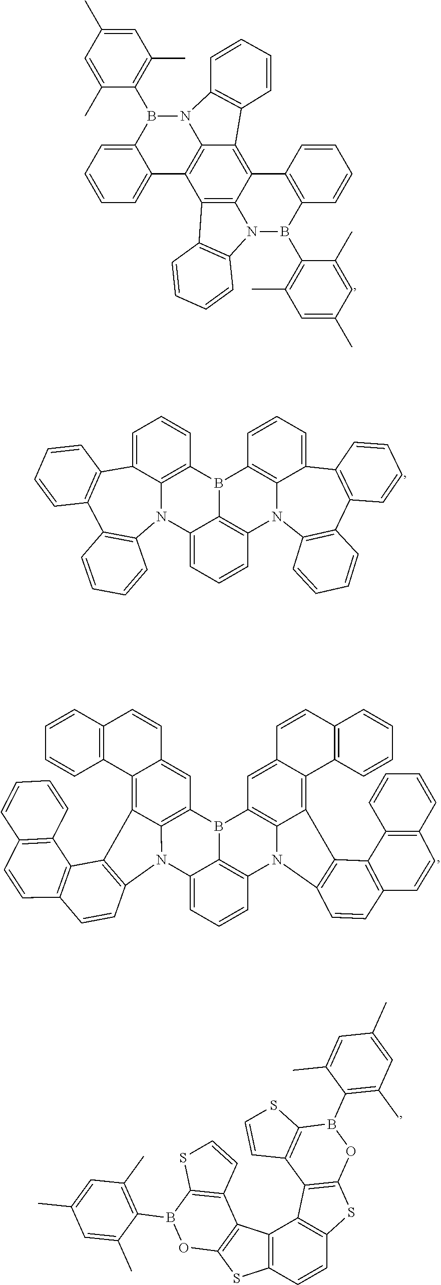

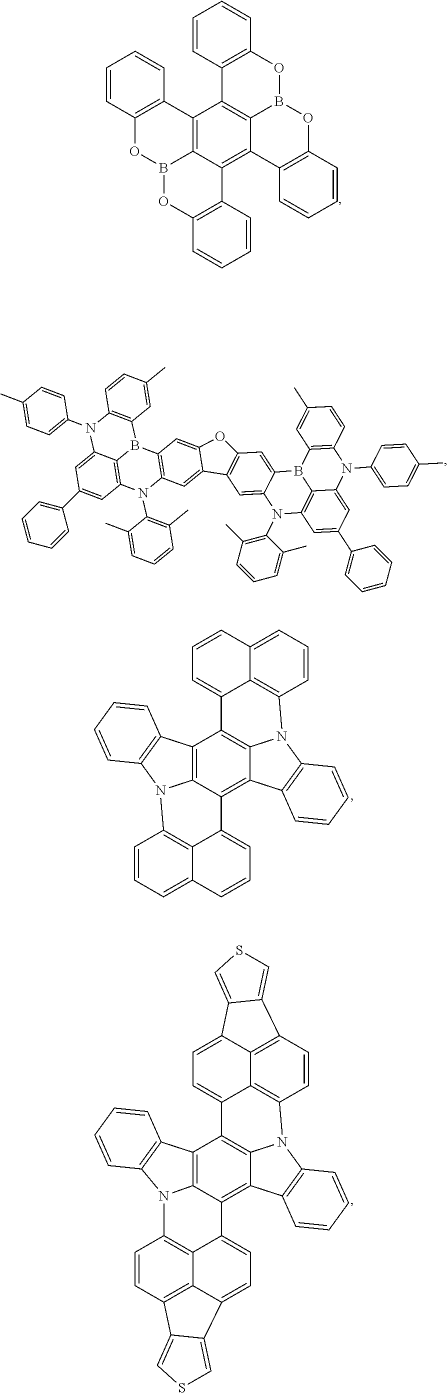

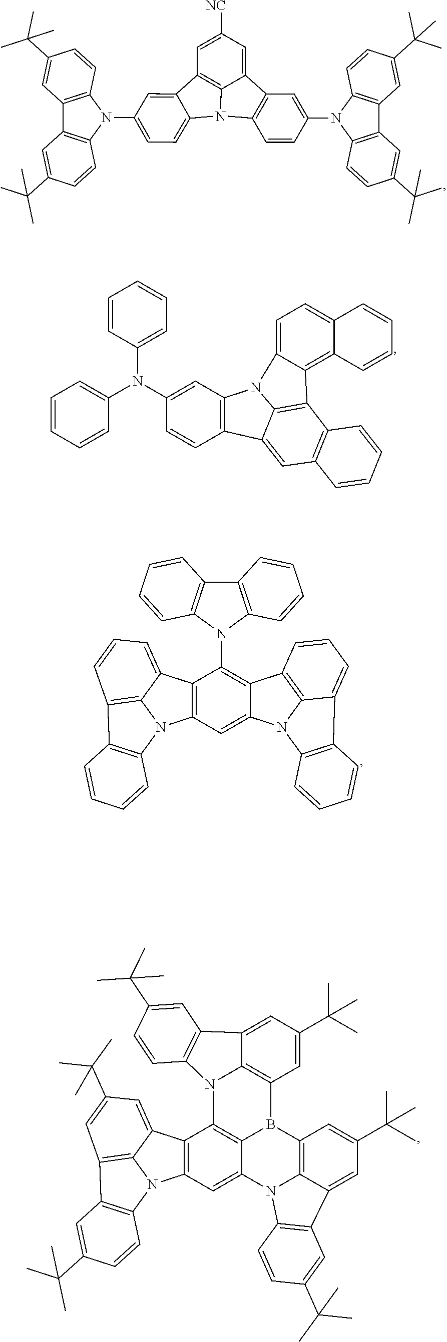

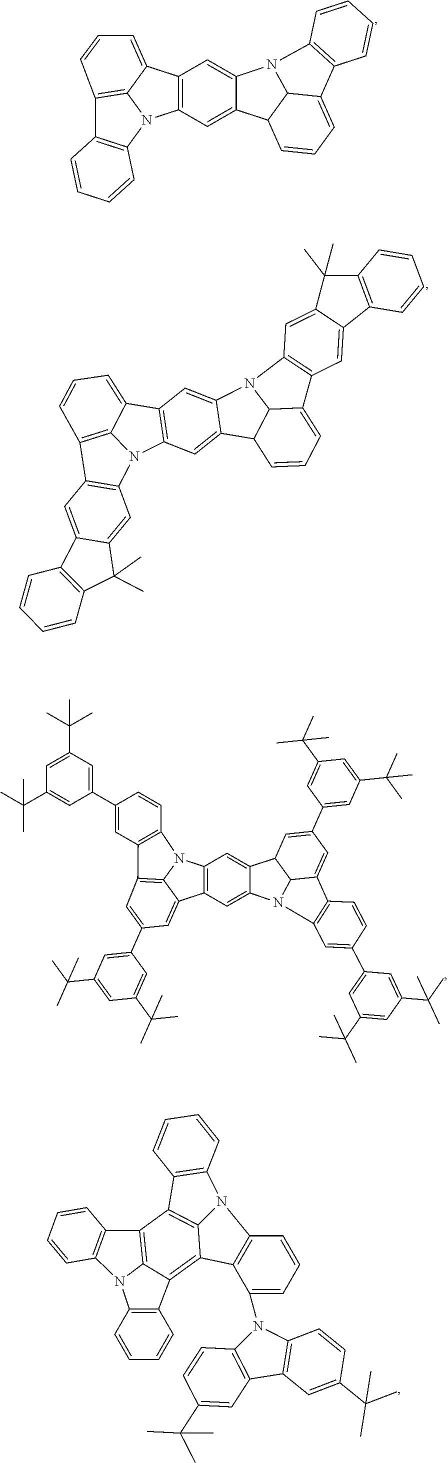 [SMILES: C1=CC2c3cc4c(cc3N3c5ccccc5C(=C1)C23)c1cccc2c3ccccc3n4c21 ; CC1(C)c2ccccc2-c2cc3c(cc21)N1c2cc4c5cccc6c7cc8c(cc7n(c4cc2C2C=CC=C3C21)c56)C(C)(C)c1ccccc1-8 ; CC(C)(C)c1cc(C2=CC3c4cc5c(cc4N4c6cc(-c7cc(C(C)(C)C)cc(C(C)(C)C)c7)ccc6C(=C2)C34)c2cc(-c3cc(C(C)(C)C)cc(C(C)(C)C)c3)cc3c4cc(-c6cc(C(C)(C)C)cc(C(C)(C)C)c6)ccc4n5c32)cc(C(C)(C)C)c1 ; CC(C)(C)c1ccc2c(c1)c1cc(C(C)(C)C)ccc1n2-c1cccc2c1c1c3c4ccccc4n4c5ccccc5c(c5c6ccccc6n2c51)c34]

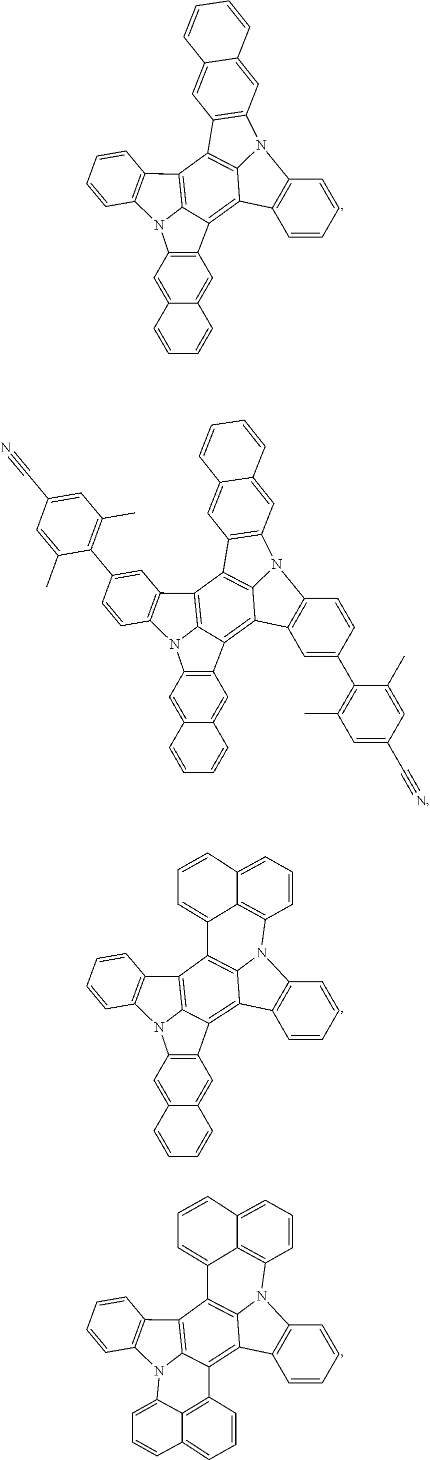

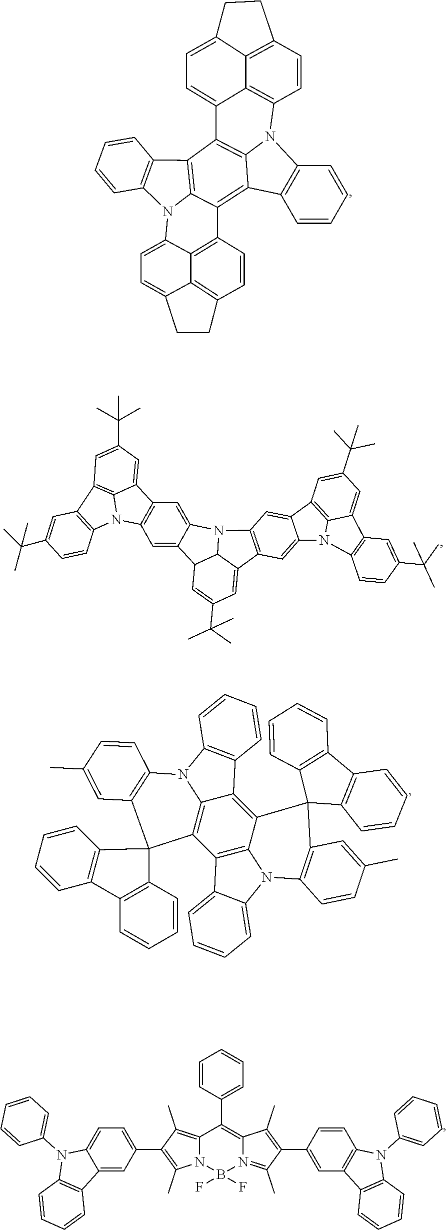

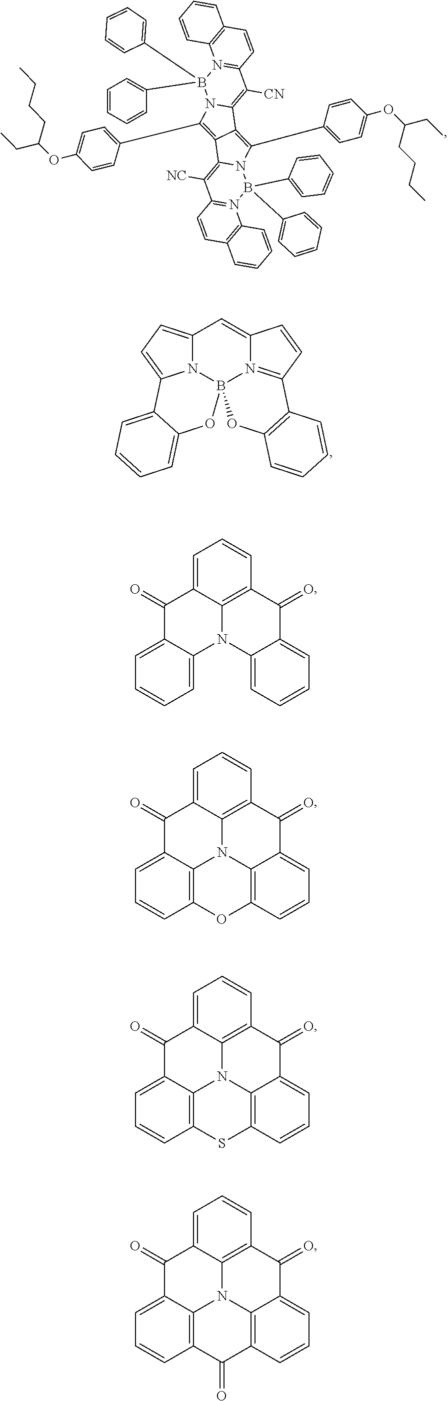

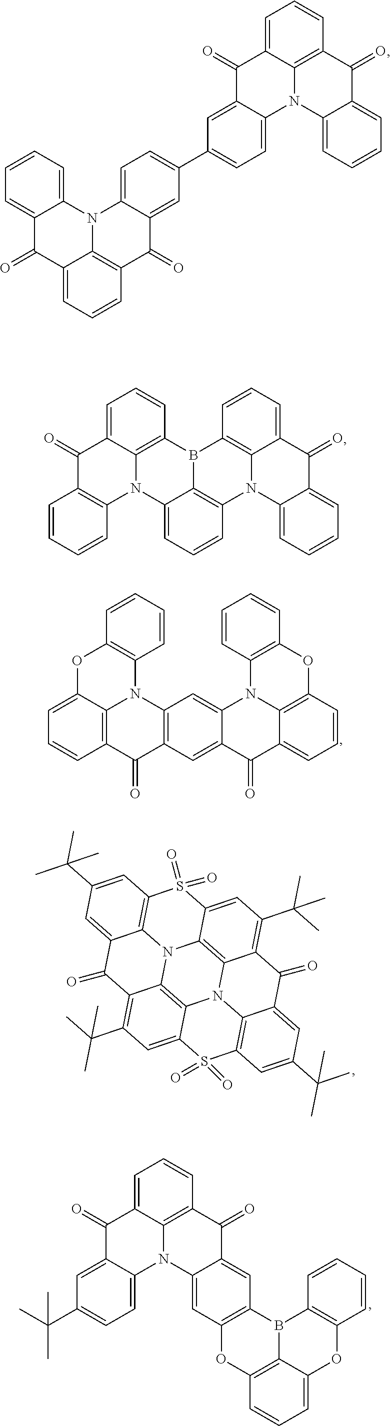

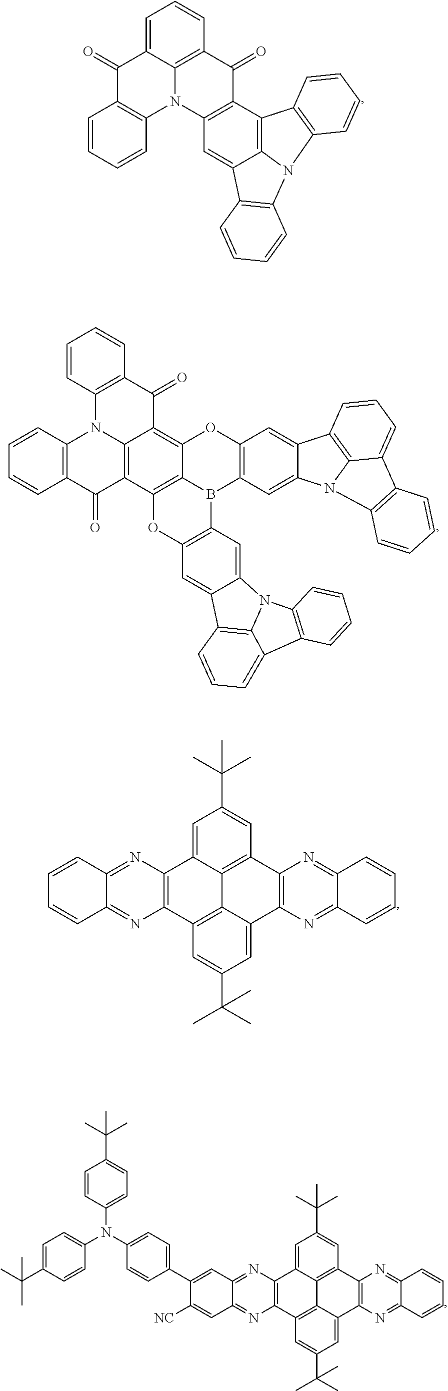 [SMILES: O=c1c2ccccc2n2c3cc4c5ccccc5n5c6ccccc6c(c3c(=O)c3cccc1c32)c45 ; O=c1c2ccccc2n2c3ccccc3c(=O)c3c4c5c(c1c32)Oc1cc2c3cccc6c7ccccc7n(c2cc1B5c1cc2c(cc1O4)c1cccc4c5ccccc5n2c41)c63 ; CC(C)(C)c1cc2c3nc4ccccc4nc3c3cc(C(C)(C)C)cc4c5nc6ccccc6nc5c(c1)c2c34 ; CC(C)(C)c1ccc(N(c2ccc(-c3cc4nc5c6cc(C(C)(C)C)cc7c8nc9ccccc9nc8c8cc(C(C)(C)C)cc(c5nc4cc3C#N)c8c76)cc2)c2ccc(C(C)(C)C)cc2)cc1]

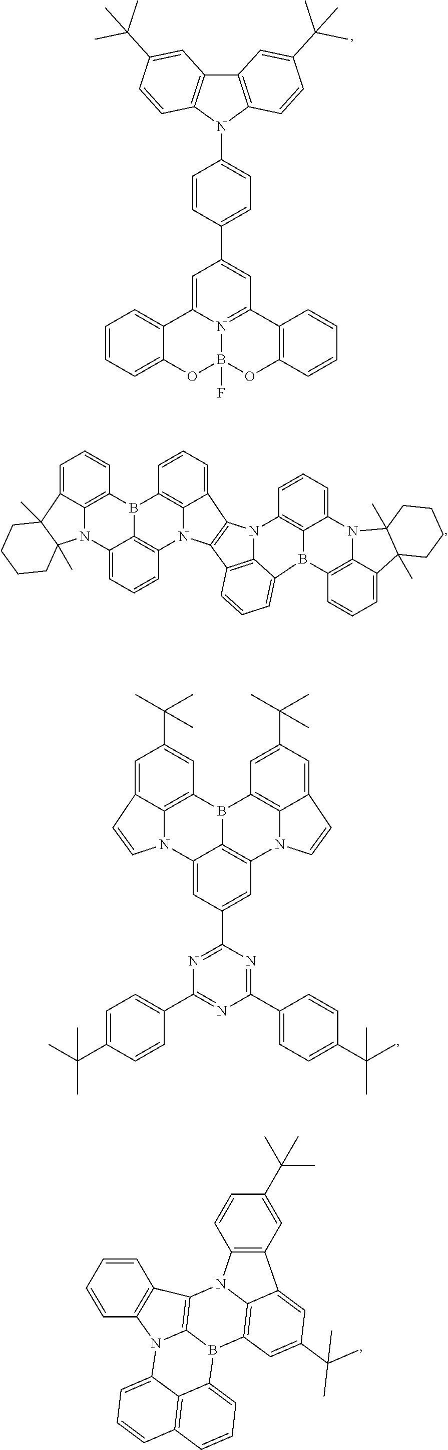

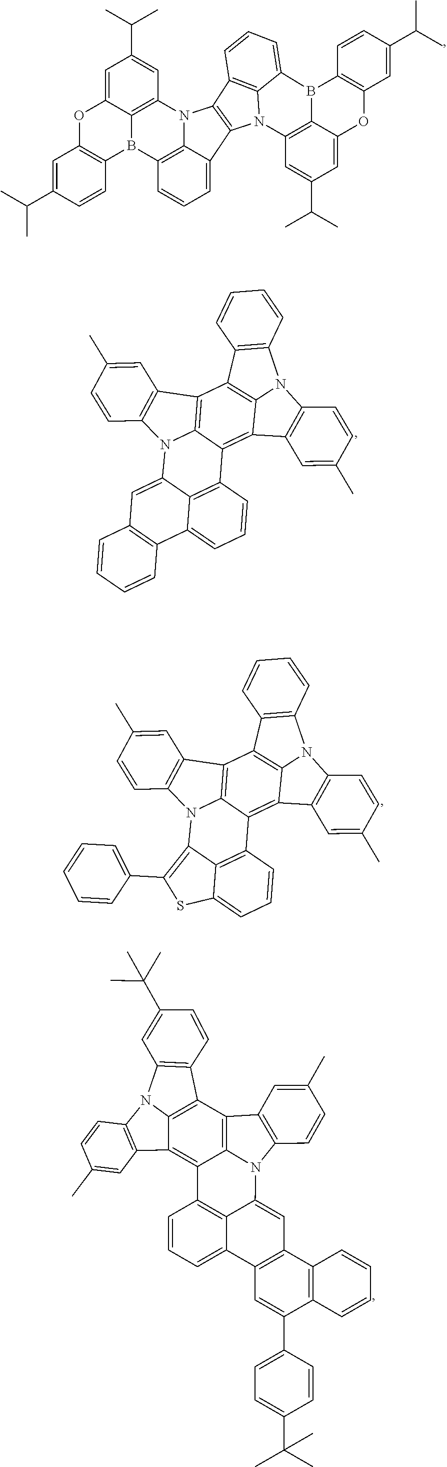

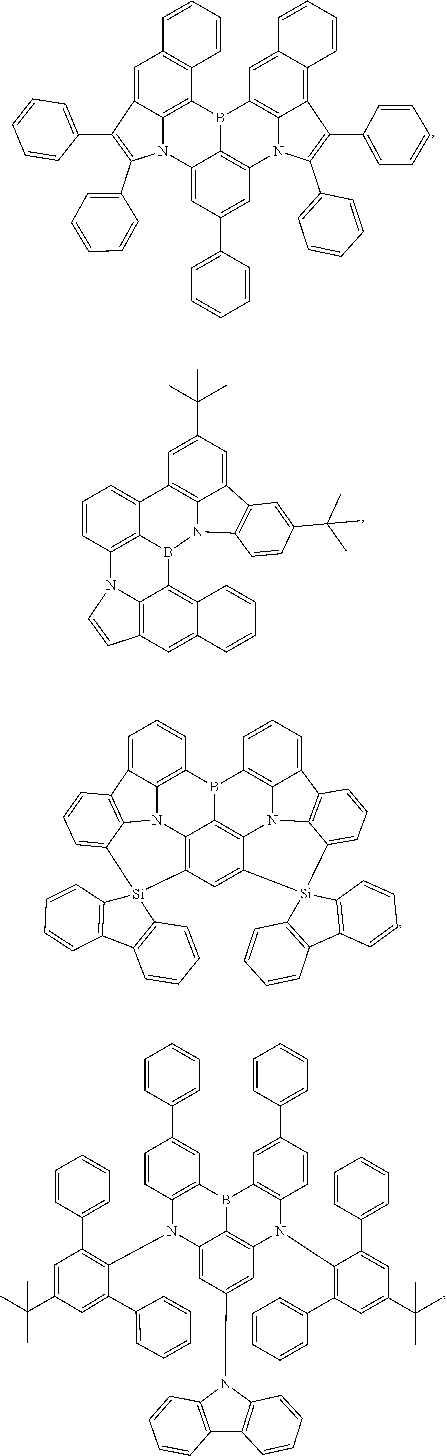

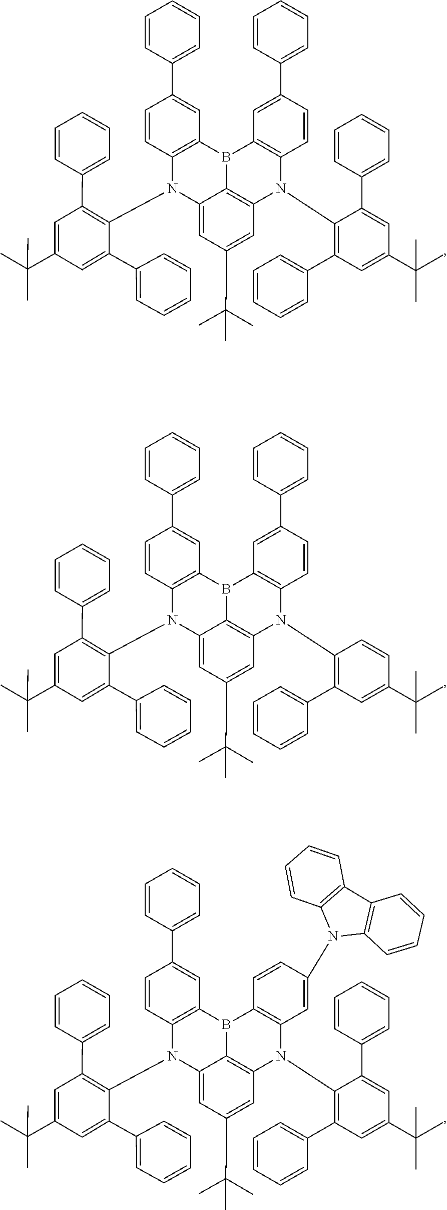

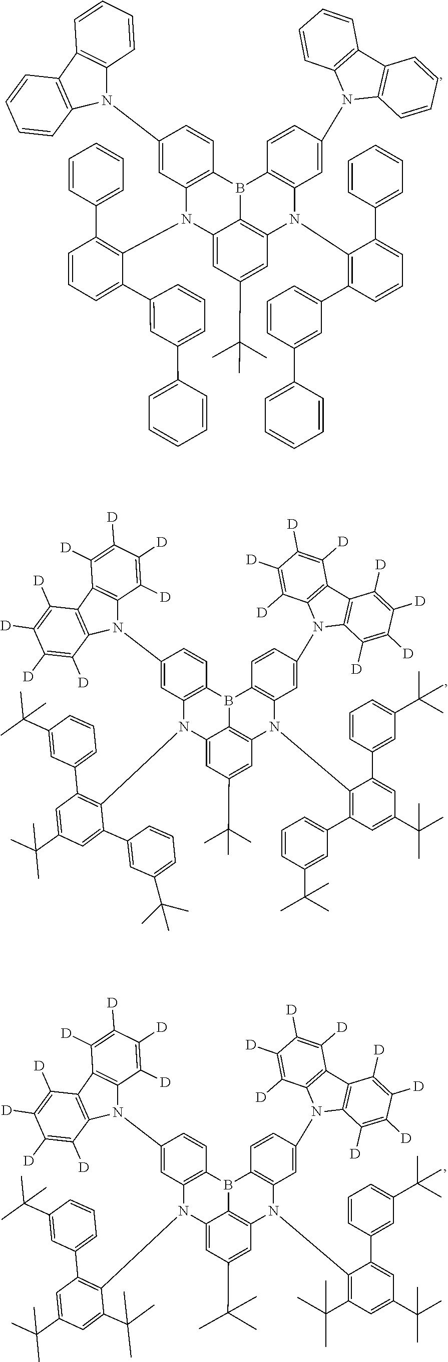

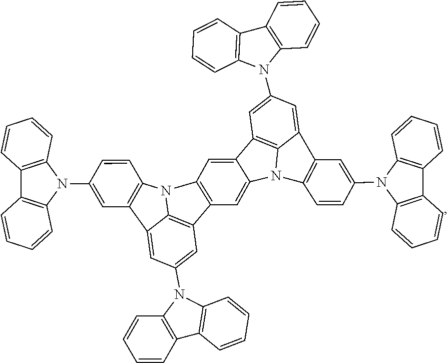 [SMILES: c1ccc2c(c1)c1ccccc1n2-c1ccc2c(c1)c1cc(-n3c4ccccc4c4ccccc43)cc3c4cc5c(cc4n2c13)c1cc(-n2c3ccccc3c3ccccc32)cc2c3cc(-n4c6ccccc6c6ccccc64)ccc3n5c21]

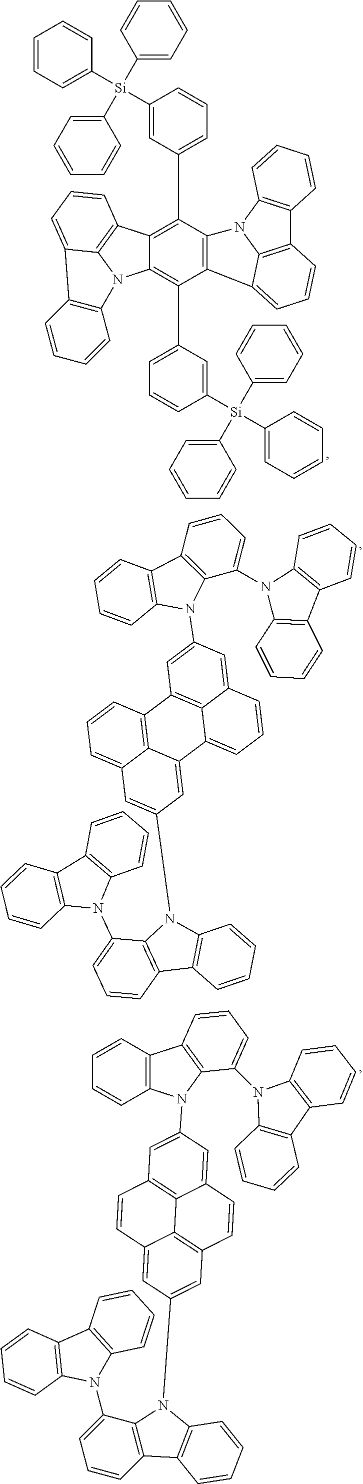

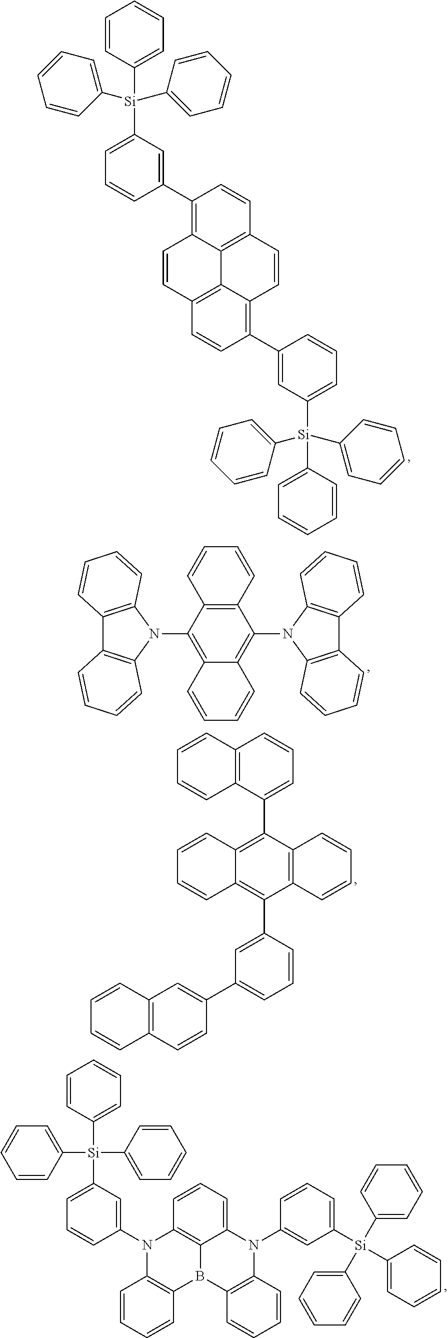

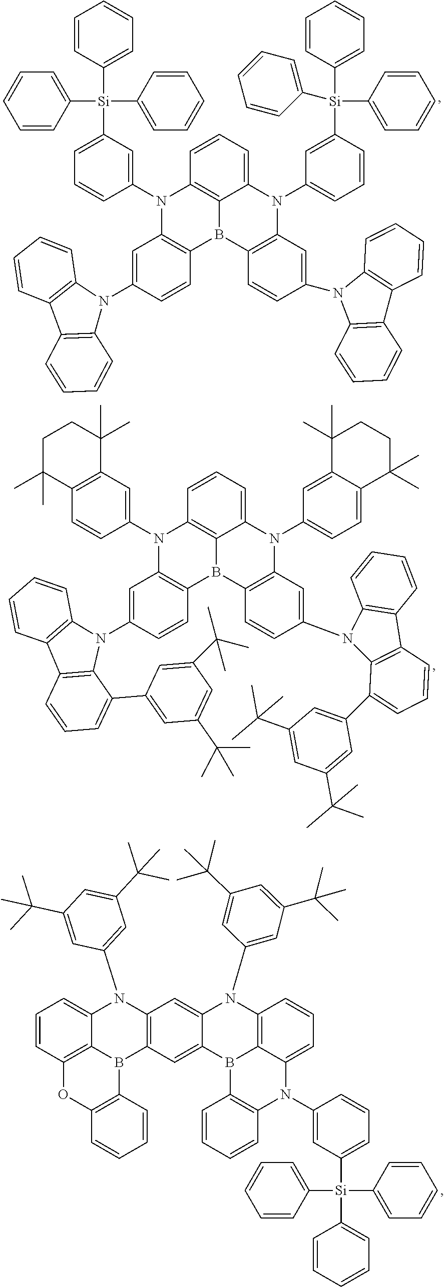

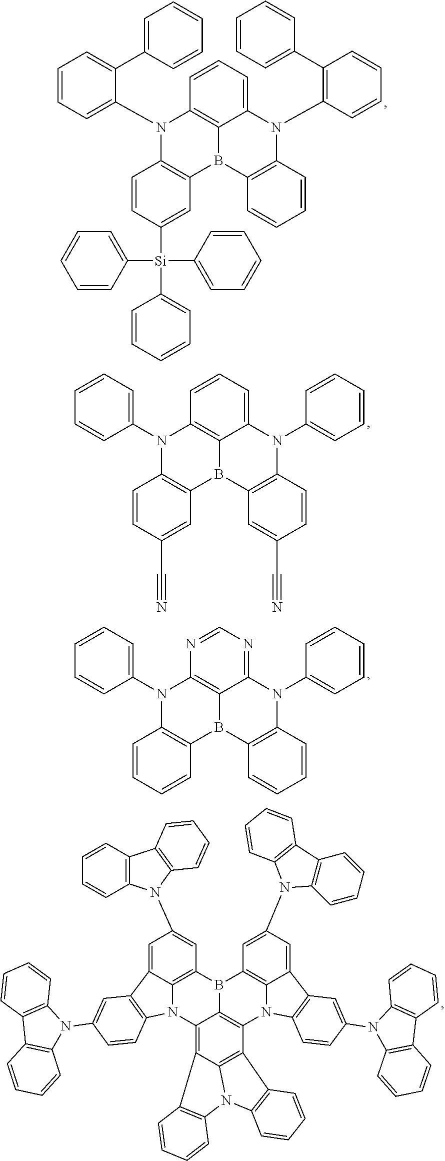

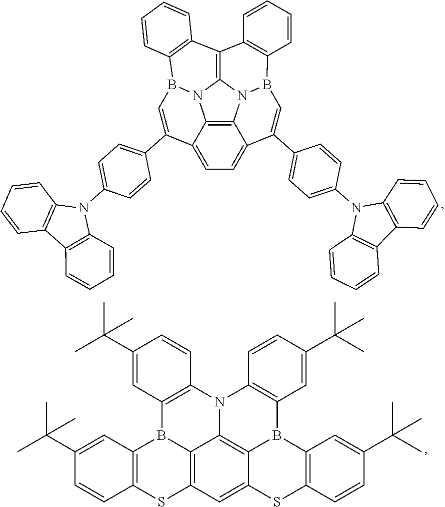

aza-substituted variants thereof, fully or partially deuterated variants thereof, and combinations thereof.

In some of the above embodiments, any carbon ring atoms up to maximum of a total number of three, together with their substitutents, in each phenyl ring of any of above structures can be replaced with N.

In some embodiments, the acceptor compound comprises a fused ring system having at least five to fifteen 5-membered and/or 6-membered aromatic rings. In some embodiments, the acceptor compound has a first group and a second group with the first group not overlapping with the second group; wherein at least 80% of the singlet excited state population of the lowest singlet excitation state are localized in the first group; and wherein at least 80%, 85%, 90%, or 95% of the triplet excited state population of the lowest triplet excitation state are localized in the second group.

In some embodiments, an acceptor has a HOMO energy and a LUMO energy,
wherein,
High HOMO energy is the highest HOMO energy among all materials in the emissive region;
Low LUMO energy is the lowest LUMO energy among all materials in the emissive region;
ΔE is the energy gap between the High HOMO energy and the Low LUMO energy;
when the acceptor is a singlet fluorescent emitter, then E_{S} is singlet energy S₁ of the acceptor, which is the lowest S₁ energy among all materials in the organic emissive region;
when the acceptor is a doublet fluorescent emitter, then E_{S} is doublet energy D₁ of the acceptor, which is the lowest D₁ energy among all materials in the organic emissive region;
a≤E_{S}−ΔE≤b, wherein a is from 0.00 to 0.15 eV, and b is from 0.05 to 0.45 eV; and
wherein root mean squared function (RMSD) value for the emission spectrum of the OLED and an emission spectrum of a reference OLED, whose organic emissive layer consists of the acceptor material and an inert host, is not greater than 0.05,
wherein RMSD value is a single value that represents the average difference between the emission spectrum of the OLED and the emission spectrum of a reference OLED at all wavelengths obtained by the following equation:

$$R M S D = \sqrt{\frac{1}{n} {\sum }_{1}^{n} {( {I}_{1} ( λ ) - {I}_{2} ( λ ) )}^{2}} ,$$

wherein n is the number of points on the two emission spectrums being compared, and I₁ and I₂ are the normalized intensity spectrums as a function of wavelength, k.

In some embodiments, the emissive region further comprises a first host. In some embodiments, the sensitizer compound forms an exciplex with the first host in the OLED at room temperature. In some embodiments, the first host has a LUMO energy that is lower than the LUMO energies of the sensitizer compound and the acceptor compound in the emissive region. In some embodiments, the first host has a HOMO energy that is lower than the HOMO energies of the sensitizer compound and the acceptor compound in the emissive region. In some embodiments, the first host has a HOMO energy that is higher than the HOMO energies of the sensitizer compound and the acceptor compound in the emissive region. In some embodiments, the first host has a HOMO energy that is higher than the HOMO energy of at least one of the sensitizer compound and the acceptor compound in the emissive region.

In some embodiments, the emissive region further comprises a second host. In some embodiments, the first host forms an exciplex with the second host in the OLED at room temperature. In some embodiments, the S-T₁ energy gap in the exciplex formed by the first host and the second host is less than 0.4, 0.3, 0.2, or 0.1 eV. In some embodiments, the T₁ energy of exciplex is greater than 2.5, 2.6, 2.7, or 2.8 eV. In some embodiments, the concentrations of the first and second hosts in the layer or layers containing the first and second host are greater than the concentrations of the sensitizer compound and the acceptor compound in the layer or layers containing the sensitizer compound and the acceptor compound. In some embodiments, the concentrations of the first and second hosts in the layer or layers containing the first and second host are greater than the concentrations of the acceptor compound in the layer or layers containing the sensitizer compound and the acceptor compound.

In some embodiments, the S₁ energy of the first host is greater than that of the acceptor compound. In some embodiments, T₁ energy of the first host is greater than that of the sensitizer compound. In some embodiments, the sensitizer compound has a HOMO energy that is greater than that of the acceptor compound. In some embodiments, the second host has a HOMO level that is shallower than that of the acceptor compound. In some embodiments, the HOMO level of the acceptor compound is deeper than at least one selected from the sensitizer compound and the first host.

In some embodiments, the first host and/or the second host comprises at least one chemical group selected from the group consisting of triphenylene, carbazole, indolocarbazole, dibenzothiophene, dibenzofuran, dibenzoselenophene, 5λ²-benzo[d]benzo[4,5]imidazo[3,2-a]imidazole, 5,9-dioxa-13b-boranaphtho[3,2,1-de]anthracene, azaborinine, oxaborinine, dihydroacridine, xanthene, dihydrobenzoazasiline, dibenzooxasiline, phenoxazine, phenoxathiine, phenothiazine, dihydrophenazine, fluorene, naphthalene, anthracene, phenanthrene, phenanthroline, benzoquinoline, quinoline, isoquinoline, quinazoline, pyrimidine, pyrazine, pyridine, triazine, boryl, silyl, aza-triphenylene, aza-carbazole, aza-indolocarbazole, aza-dibenzothiophene, aza-dibenzofuran, aza-dibenzoselenophene, aza-5λ²-benzo[d]benzo[4,5]imidazo[3,2-a]imidazole, and aza-(5,9-dioxa-13b-boranaphtho[3,2,1-de]anthracene. In some embodiments the first host and the second host are both organic compounds.

In some embodiments, at least one of the first host and the second host is a metal complex.

In some embodiments, each of the first host and/or the second host is independently selected from the HOST GROUP 1 consisting of:

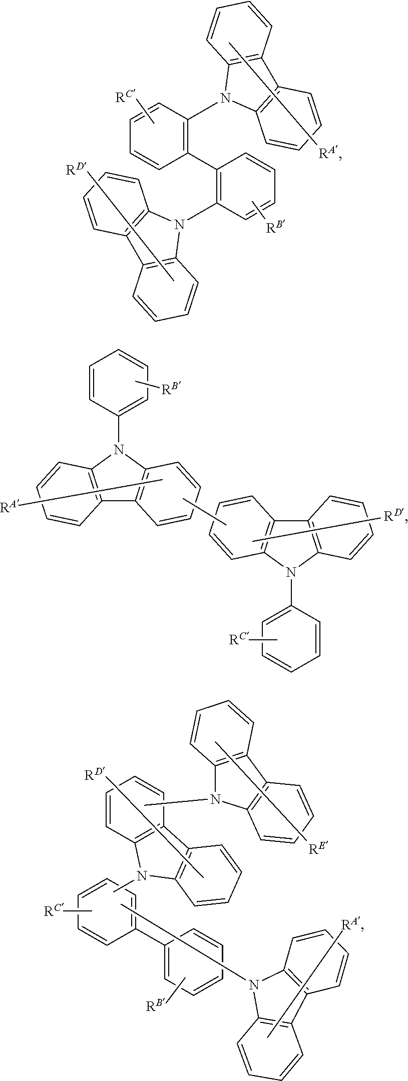

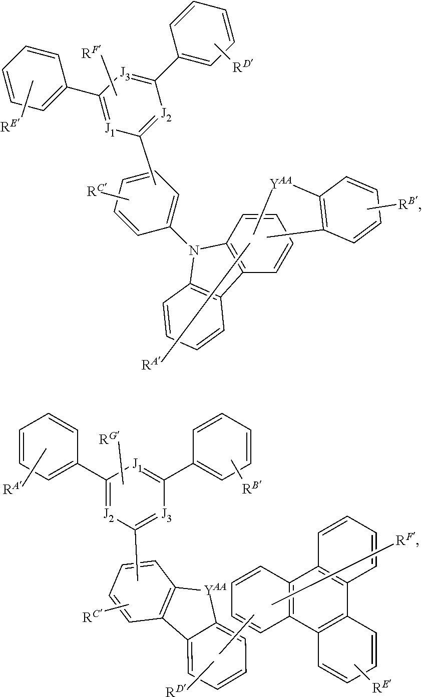

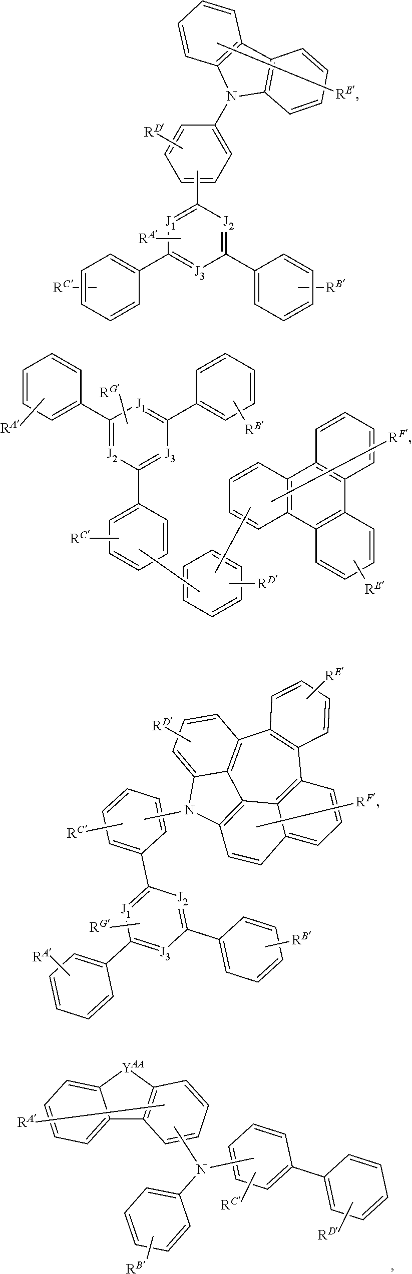

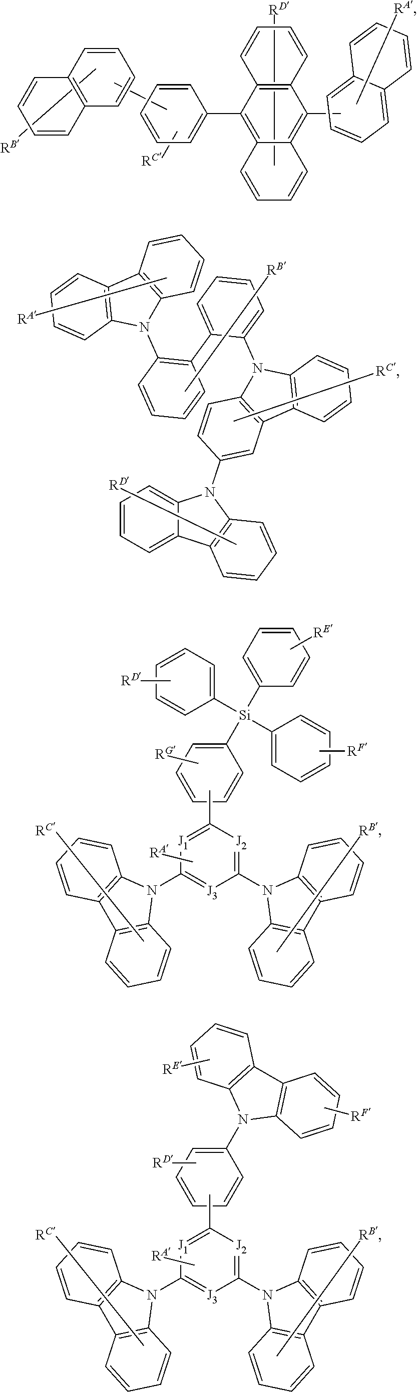

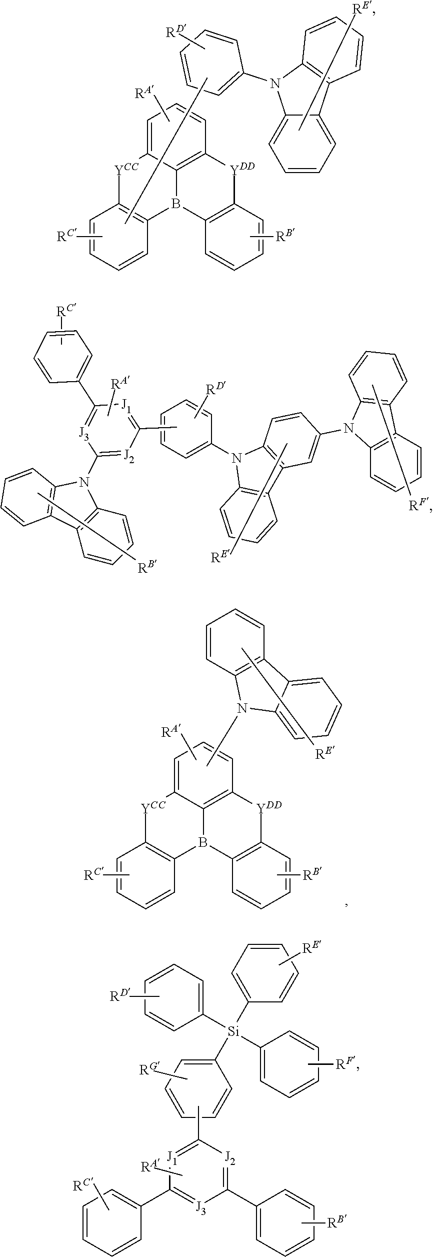

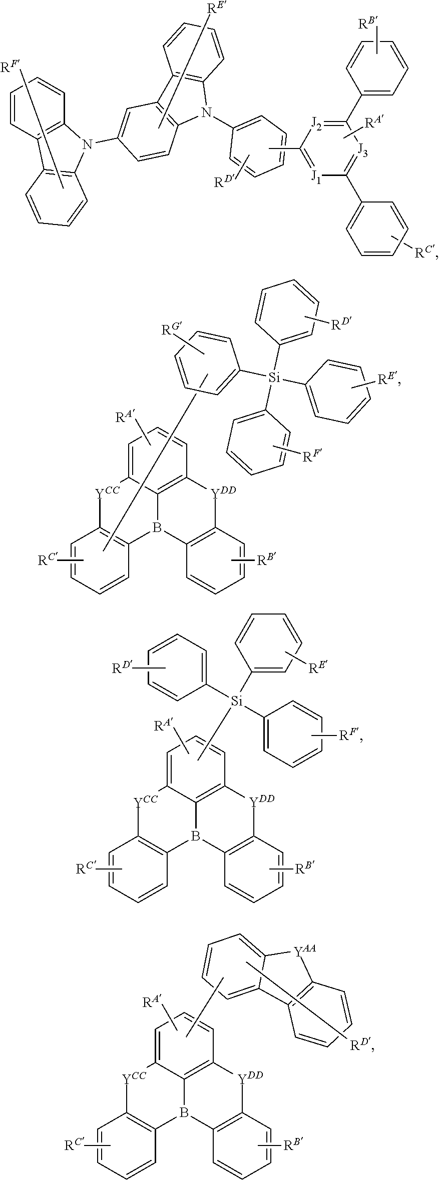

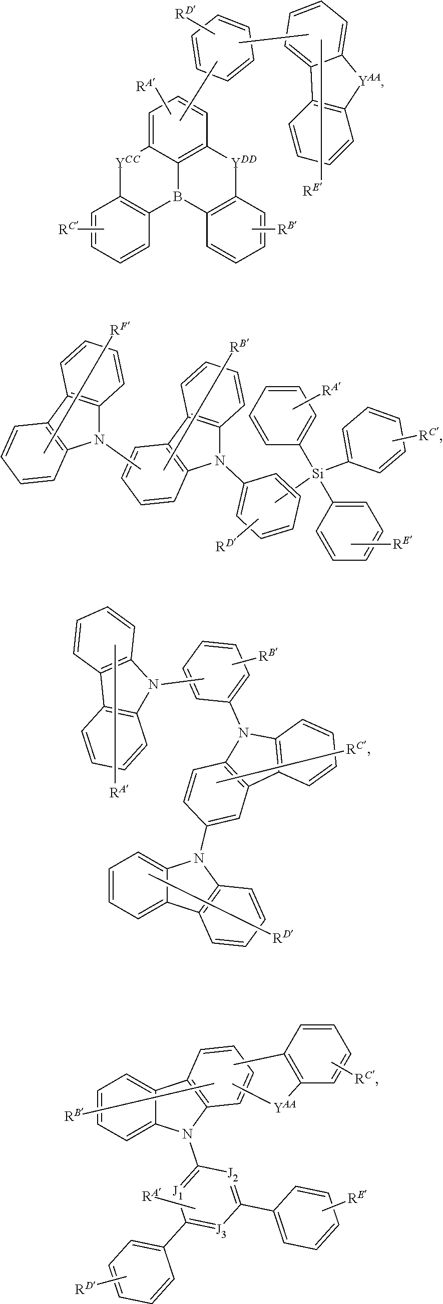

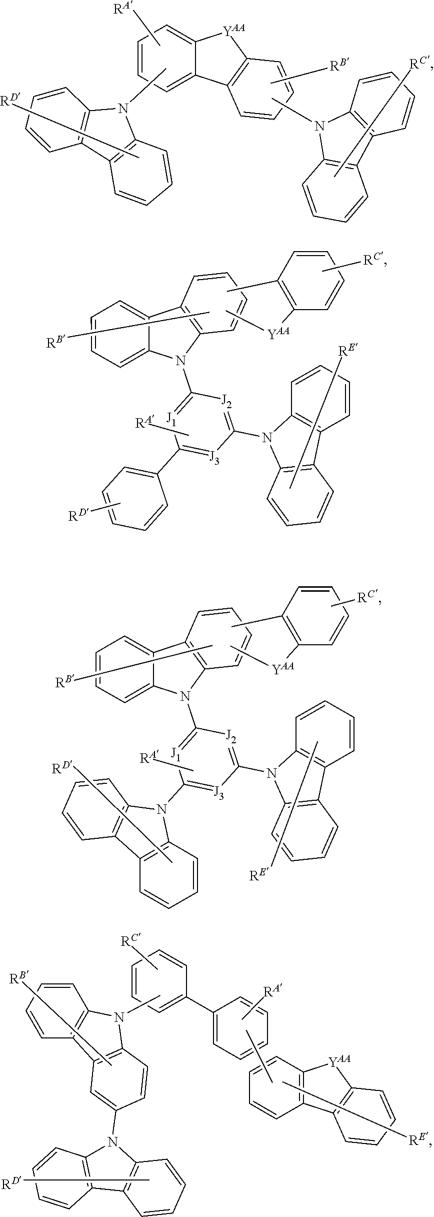

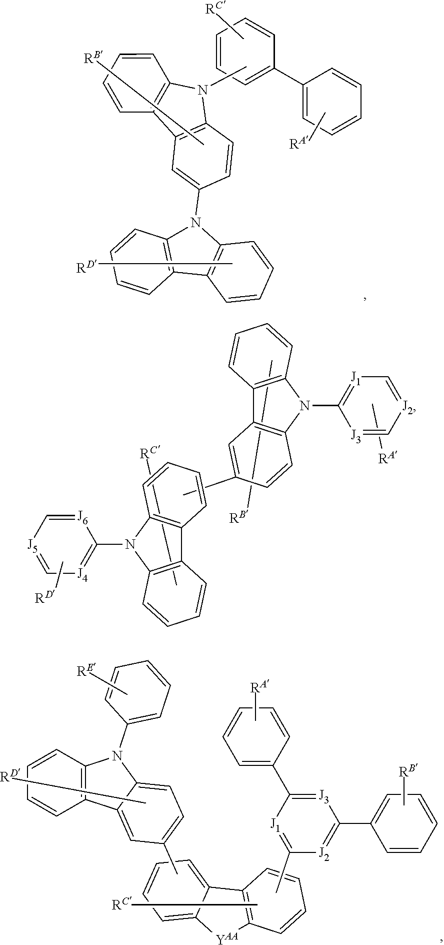

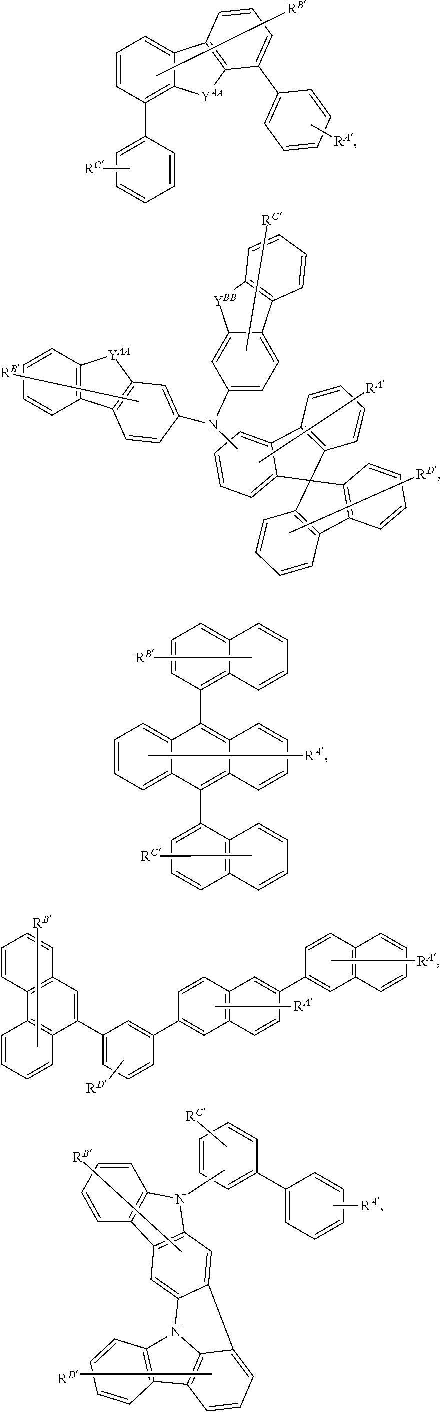

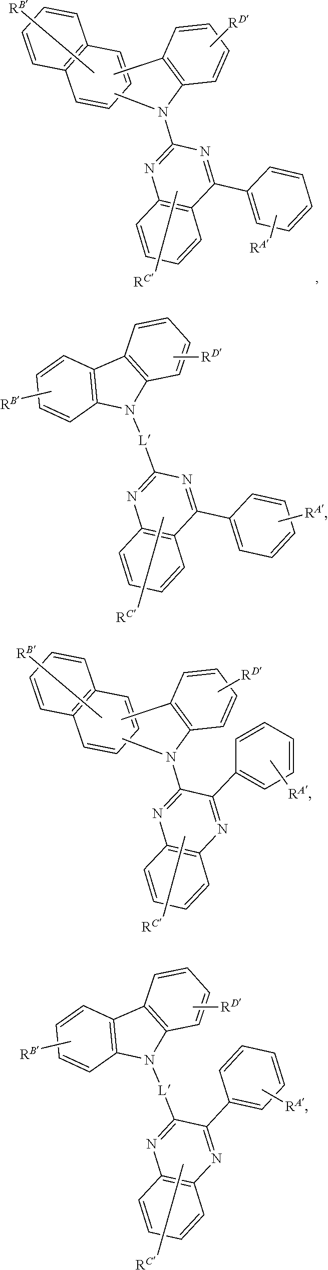

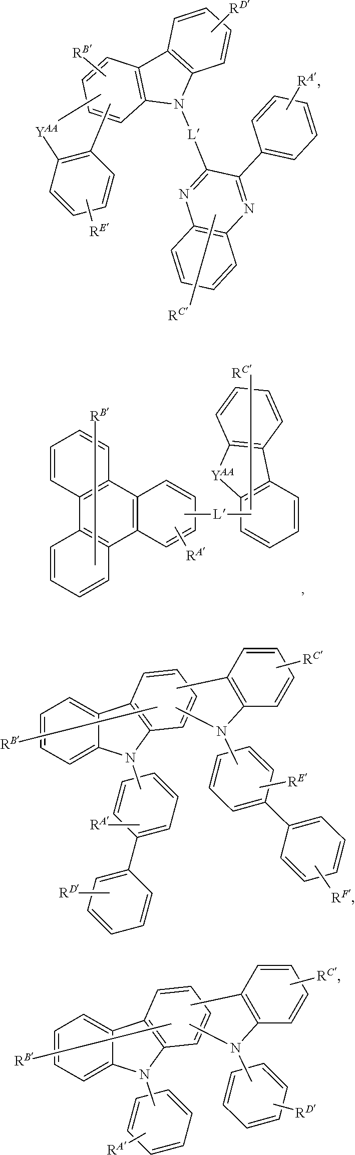

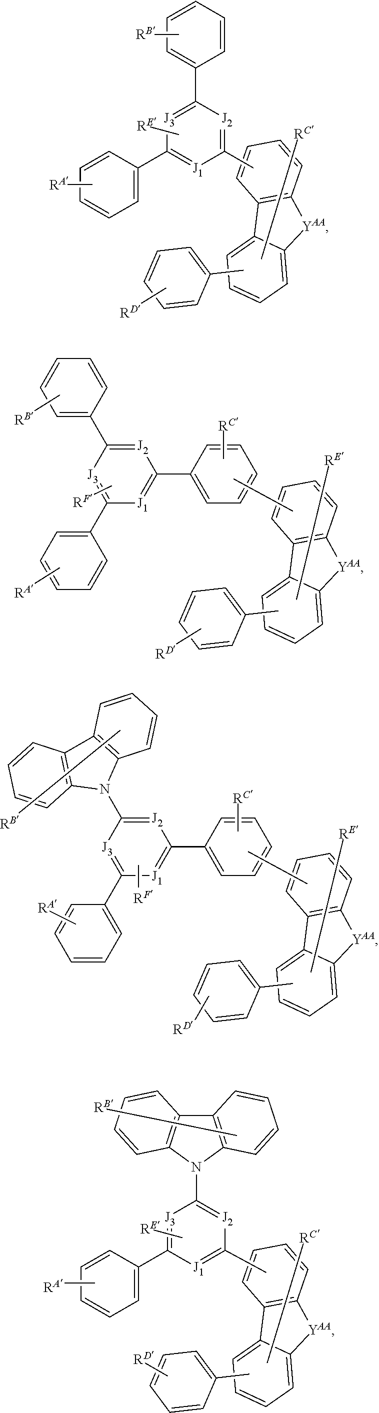

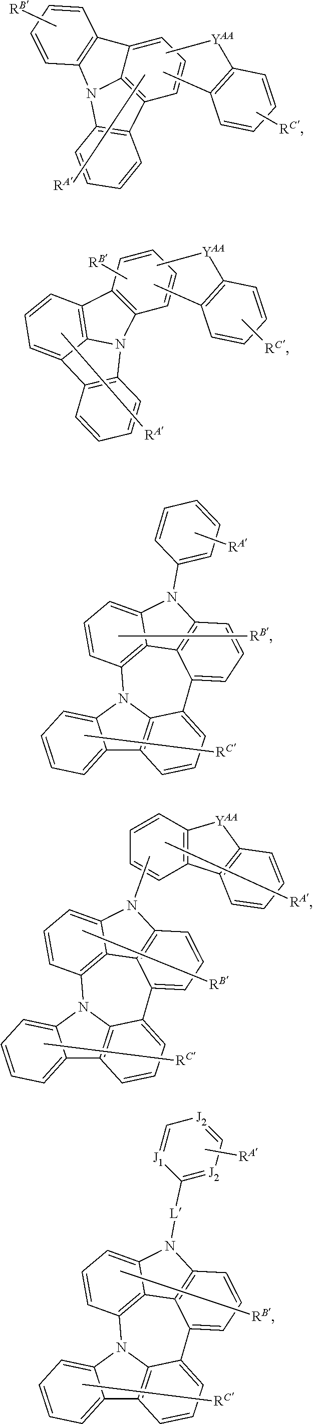

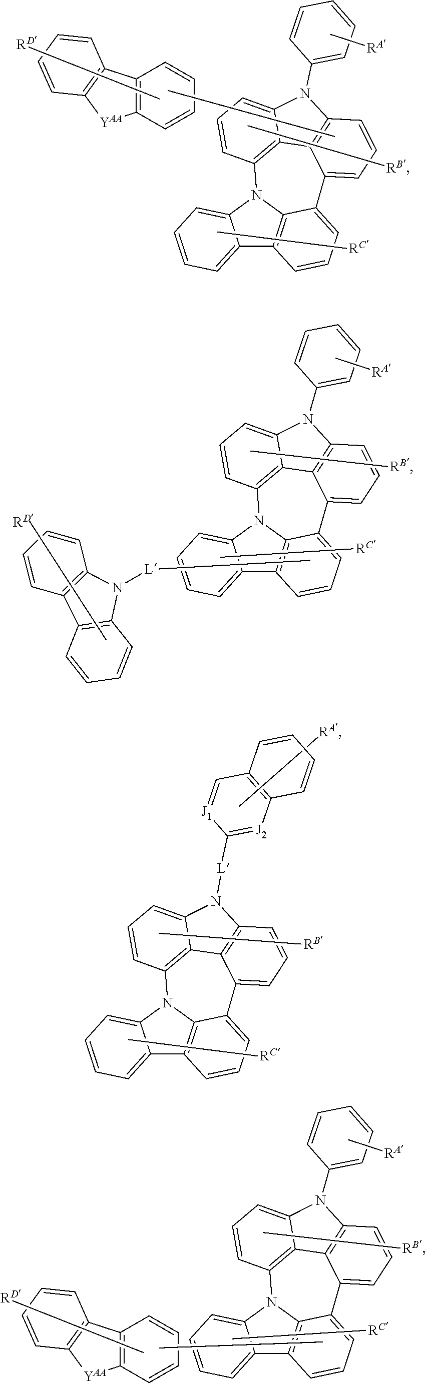

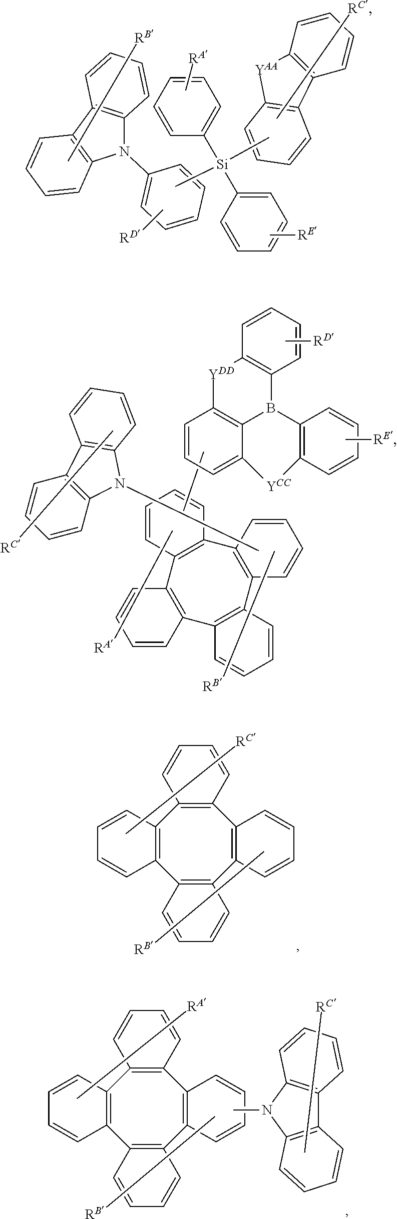

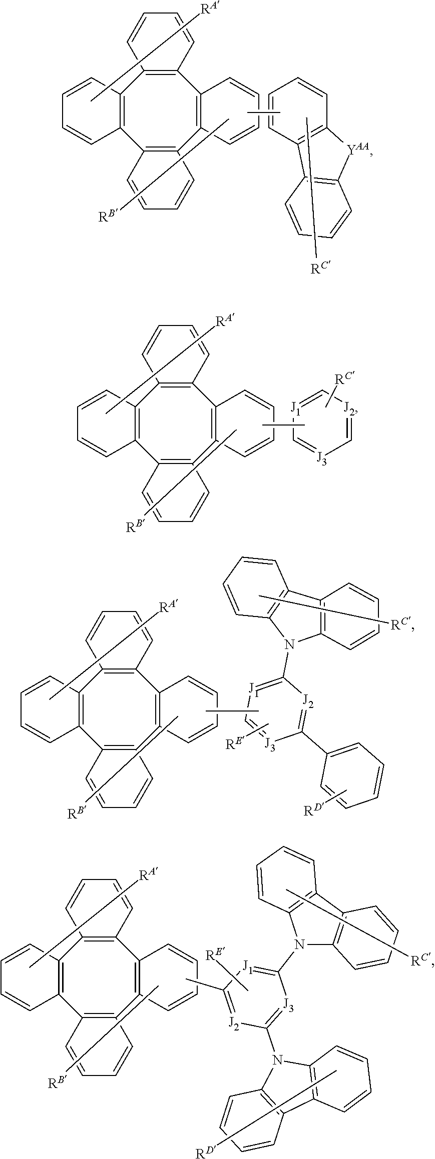

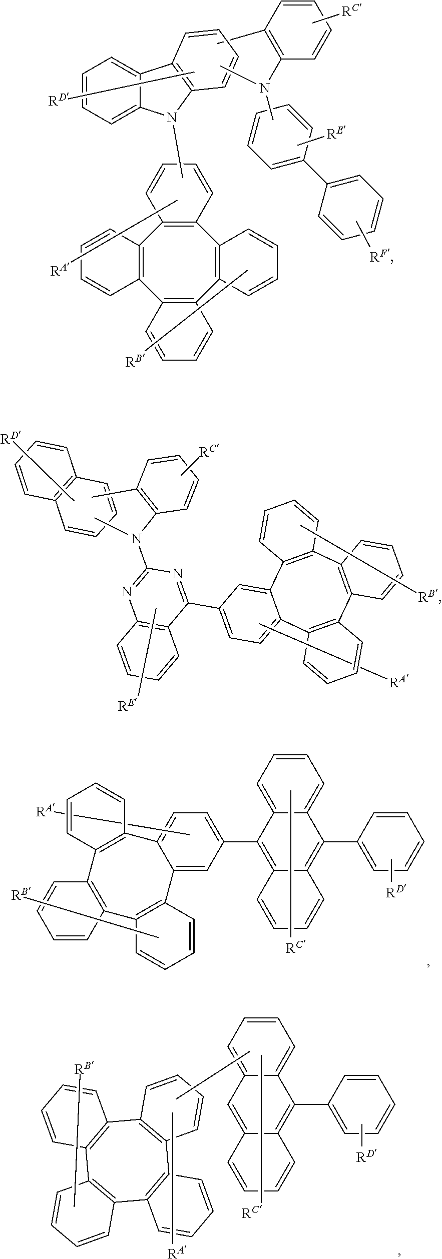

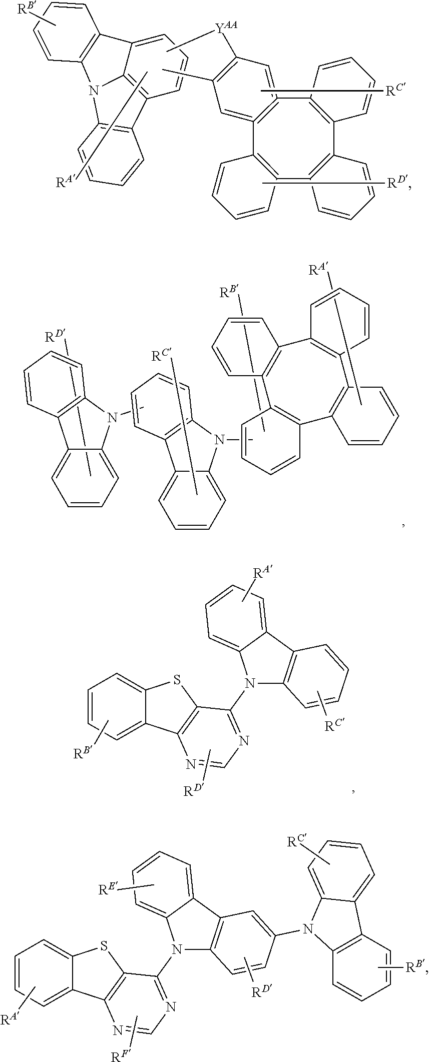

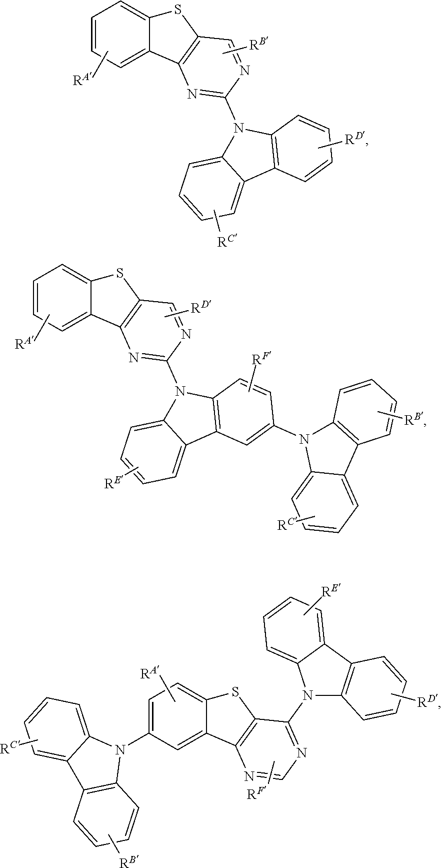

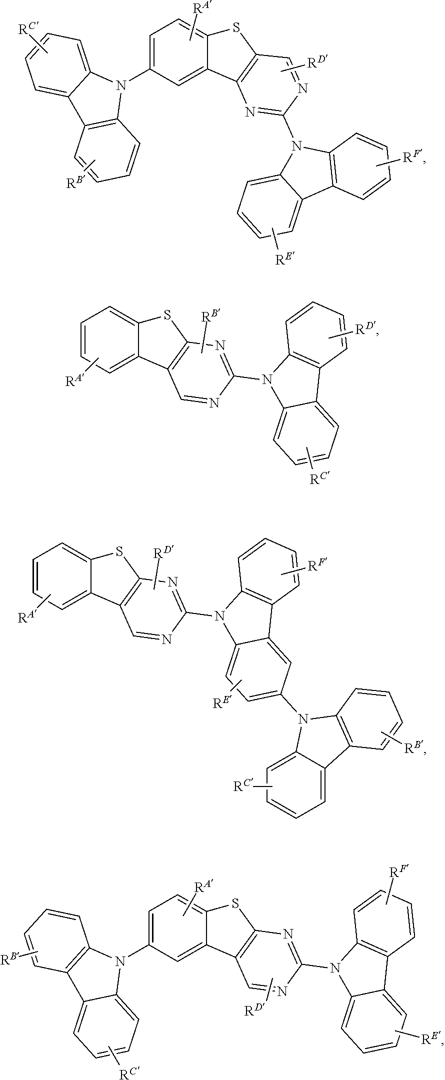

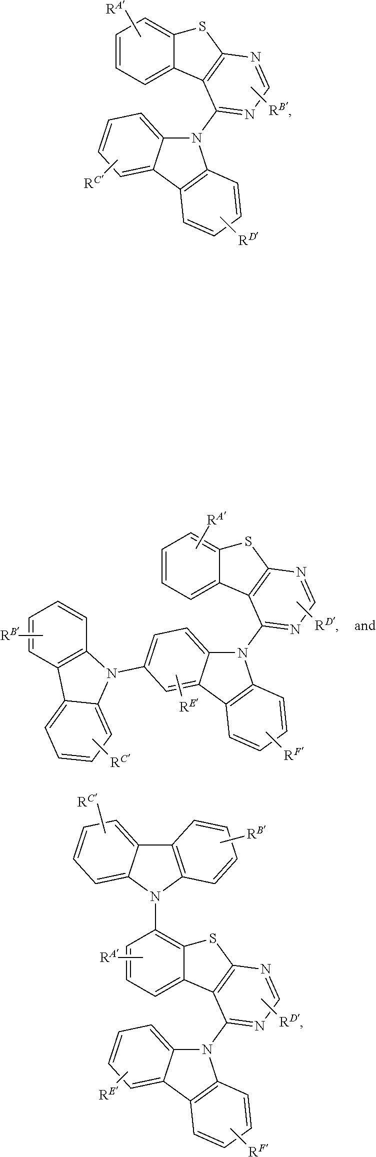

wherein:
each of J₁ to J₆ is independently C or N;
L′ is a direct bond or an organic linker;
each Y^{AA}, Y^{BB}, Y^{CC} and Y^{DD} is independently selected from the group consisting of absent a bond, direct bond, O, S, Se, CRR′, SiRR′, GeRR′, NR, BR, BRR′;
each of R^{A′}, R^{B′}, R^{C}, R^{D′}, R^{E′}, R^{F′}, and R^{G′} independently represents mono, up to the maximum substitutions, or no substitutions;
each R, R′, R^{A′}, R^{B′}, R^{C′}, R^{D′}, R^{E′}, R^{F′}, and R^{G′} is independently a hydrogen or a substituent selected from the group consisting of the general substituents as defined herein; any two substituents can be joined or fused to form a ring; and where possible, each unsubstituted aromatic carbon atom can be replaced with one or more N to form an aza-substituted ring.

In some embodiments at least one of J₁ to J₃ are N. In some embodiments at least two of J₁ to J₃ are N. In some embodiments, all three of J₁ to J₃ are N. In some embodiments, each Y^{CC} and Y^{DD} are preferably O, S, and SiRR′, more preferably O, or S. In some embodiments, at least one unsubstituted aromatic carbon atom is replaced with N to form an aza-ring.

In some embodiments, L′ is an organic linker selected from the group consisting of BR, BRR′, NR, PR, P(O)R, O, S, Se, C═O, C═S, C═Se, C═NR, C═CRR′, S═O, SO₂, CR, CRR′, SiRR′, GeRR′, alkylene, cycloalkyl, aryl, cycloalkylene, arylene, heteroarylene, and combinations thereof.

To reduce the amount of Dexter energy transfer between the sensitizer compound and the acceptor compound, it would be preferable to have a large distance between the center of mass of the sensitizer compound and the center of mass of the closest neighboring acceptor compound in the emissive region. Therefore, in some embodiments, the distance between the center of mass of the acceptor compound and the center of mass of the sensitizer compound is at least 2, 1.5, 1.0, or 0.75 nm.

Preferred acceptor/sensitizer VDR combination (A): In some embodiments, it is preferable for the VDR of the acceptor to be less than 0.33 in order to reduce the coupling of the transition dipole moment of the emitting acceptor to the plasmon modes, compared to an isotropic emitter, in order to achieve a higher outcoupling efficiency. In some cases, when the VDR of the acceptor is less than 0.33, it would be preferable for the VDR of the sensitizer to be less than 0.33 in order to improve the coupling of the transition dipole moments of the sensitizer and acceptor to optimize the Forster energy transfer rate. Accordingly, in some embodiments of the inventive OLED, the acceptor compound in the inventive OLED exhibits a VDR value equal to or less than 0.33, 0.30, 0.25, 0.2, 0.15, 0.10, 0.08, or 0.05 when the VDR is measured with an emissive thin film test sample that has the acceptor compound as the only emitter; and the sensitizer compound in the inventive OLED exhibits a VDR value equal to or less than 0.33, 0.30, 0.25, 0.2, 0.15, 0.10, 0.08, or 0.05 when the VDR is measured with an emissive thin film test sample that has the sensitizer compound as the only emitter.

Preferred acceptor/sensitizer VDR combination (B): In some embodiments, it is preferable for the VDR of the acceptor to be less than 0.33 in order to reduce the coupling of the transition dipole moment of the emitting acceptor to the plasmon modes compared to an isotropic emitter in order to achieve a higher outcoupling efficiency. In some cases, when the VDR of the acceptor is less than 0.33, it would be preferable to minimize the intermolecular interactions between the sensitizer and acceptor to decrease the degree of Dexter quenching. By changing the molecular geometry of the sensitizer to reduce the intermolecular interactions, it may be preferable to have a sensitizer with a VDR greater than 0.33. Accordingly, in some embodiments of the inventive OLED, the acceptor compound in the inventive OLED exhibits a VDR value equal to or less than 0.33, 0.30, 0.25, 0.2, 0.15, 0.10, 0.08, or 0.05 when the VDR is measured with an emissive thin film test sample that has the acceptor compound as the only emitter; and the sensitizer compound in the inventive OLED exhibits a VDR value larger than 0.33, 0.4, 0.5, 0.6, or 0.7 when the VDR is measured with an emissive thin film test sample that has the sensitizer compound as the only emitter.

Preferred acceptor/sensitizer VDR combination (C): In some embodiments, it is preferable for the VDR of the acceptor to be greater than 0.33 in order to increase the coupling of the transition dipole moment of the acceptor to the plasmon modes compared to an isotropic emitter in order to decrease the transient lifetime of the excited states in the emissive layer. In some cases, the increased coupling to the plasmon modes can be paired with an enhancement layer in a plasmonic OLED device to improve efficiency and extend operational lifetime. In some cases, when the VDR of the acceptor is greater than 0.33, it would be preferable to minimize the intermolecular interactions between the sensitizer and acceptor to decrease the degree of Dexter quenching. By changing the molecular geometry of the sensitizer to reduce the intermolecular interactions, it may be preferable to have a sensitizer with a VDR less than 0.33. Accordingly, in some embodiments of the inventive OLED, the acceptor compound in the inventive OLED exhibits a VDR value larger than 0.33, 0.4, 0.5, 0.6, or 0.7 when the VDR is measured with an emissive thin film test sample that has the acceptor compound as the only emitter; and the sensitizer compound in the inventive OLED exhibits a VDR value equal to or less than 0.33, 0.30, 0.25, 0.2, 0.15, 0.10, 0.08, or 0.05 when the VDR is measured with an emissive thin film test sample that has the sensitizer compound as the only emitter.

Preferred acceptor/sensitizer VDR combination (D): In some embodiments, it is preferable for the VDR of the acceptor to be greater than 0.33 in order to increase the coupling of the transition dipole moment of the acceptor to the plasmon modes compared to an isotropic emitter in order to decrease the transient lifetime of the excited states in the emissive layer. In some cases, the increased coupling to the plasmon modes can be paired with an enhancement layer in a plasmonic OLED device to improve efficiency and extend operational lifetime. In some cases, when the VDR of the acceptor is greater than 0.33, it would be preferable for the VDR of the sensitizer to be greater than 0.33 in order to improve the coupling of the transition dipole moments of the sensitizer and acceptor to optimize the Forster energy transfer rate. Accordingly, in some embodiments of the inventive OLED, the acceptor compound in the inventive OLED exhibits a VDR value larger than 0.33, 0.4, 0.5, 0.6, or 0.7 when the VDR is measured with an emissive thin film test sample that has the acceptor compound as the only emitter; and the sensitizer compound in the inventive OLED exhibits a VDR value larger than 0.33, 0.4, 0.5, 0.6, or 0.7 when the VDR is measured with an emissive thin film test sample that has the sensitizer compound as the only emitter.

VDR is the ensemble average fraction of vertically oriented molecular dipoles of the light-emitting compound in a thin film sample of an emissive layer, where the orientation “vertical” is relative to the plane of the surface of the substrate (i.e., normal to the surface of the substrate plane) on which the thin film sample is formed. A similar concept is horizontal dipole ratio (HDR) which is the ensemble average fraction of horizontally oriented molecular dipoles of the light-emitting compound in a thin film sample of an emissive layer, where the orientation “horizontal” is relative to the plane of the surface of the substrate (i.e. parallel to the surface of the substrate plane) on which the thin film sample is formed. By definition, VDR+HDR=1. VDR can be measured by angle dependent, polarization dependent, photoluminescence measurements. By comparing the measured emission pattern of a photo-excited thin film test sample, as a function of polarization, to the computationally modeled pattern, one can determine VDR of the thin film test sample emission layer. For example, a modelled data of p-polarized emission is shown in FIG. 3. The modelled p-polarized angle photoluminescence (PL) is plotted for emitters with different VDRs. A peak in the modelled PL is observed in the p-polarized PL around the angle of 45 degrees with the peak PL being greater when the VDR of the emitter is higher.

To measure VDR values of the thin film test samples, a thin film test sample can be formed with the acceptor compound or the sensitizer compound (depending on whether the VDR of the acceptor compound or the sensitizer compound is being measured) as the only emitter in the thin film and a Reference Host Compound A as the host. Preferably, the Reference Host Compound A is

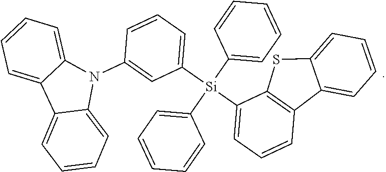 [SMILES: c1ccc([Si](c2ccccc2)(c2cccc(-n3c4ccccc4c4ccccc43)c2)c2cccc3c2sc2ccccc23)cc1]

The thin film test sample is formed by thermally evaporating the emitter compound and the host compound on a substrate. For example, the emitter compound and the host compound can be co-evaporated. In some embodiments, the doping level of the emitter compounds in the host can be from 0.1 wt. % to 50 wt. %. In some embodiments, the doping level of the emitter compounds in the host can be from 3 wt. % to 20 wt. % for blue emitters. In some embodiments, the doping level of the emitter compounds in the host can be from 1 wt. % to 15 wt. % for red and green emitters. The thickness of the thermally evaporated thin film test sample can have a thickness of from 50 to 1000 Å.

In some embodiments, the OLED of the present disclosure can comprise a sensitizer, an acceptor, and one or more hosts in the emissive region, and the preferred acceptor/sensitizer VDR combinations (A)-(D) mentioned above are still applicable. In these embodiments, the VDR values for the acceptor compound can be measured with a thin film test sample formed of the one or more hosts and the acceptor, where the acceptor is the only emitter in the thin film test sample. Similarly, the VDR values for the sensitizer compound can be measured with a thin film test sample formed of the one or more hosts and the sensitizer, where the sensitizer is the only emitter in the thin film test sample.

In the example used to generate FIG. 3, a 30 nm thick film of material with a refractive index of 1.75 and the emission is monitored in a semi-infinite medium with a refractive index of 1.75. Each curve is normalized to a photoluminescence intensity of 1 at an angle of zero degrees, which is perpendicular to the surface of the film. As the VDR of the emitter is varied, the peak around 45 degrees increases greatly. When using a software to fit the VDR of experimental data, the modeled VDR would be varied until the difference between the modeled data and the experimental data is minimized.

Because the VDR represents the average dipole orientation of the light-emitting compound in the thin film sample, even if there are additional emission capable compounds in the emissive layer, if they are not contributing to the light emission, the VDR measurement does not reflect their VDR. Further, by inclusion of a host material that interacts with the light-emitting compound, the VDR of the light-emitting compound can be modified. Thus, a light-emitting compound in a thin film sample with host material A will exhibit one measured VDR value and that same light-emitting compound in a thin film sample with host material B will exhibit a different measured VDR value. Further, in some embodiments, exciplex or excimers are desirable which form emissive states between two neighboring molecules. These emissive states may have a VDR that is different than that if only one of the components of the exciplex or excimer were emitting or present in the sample.

In some embodiments, the OLED is a plasmonic OLED. In some embodiments, the OLED is a wave-guided OLED.

In some embodiments, the OLED emits a white light at room temperature when a voltage is applied across the device.

In some embodiments, the OLED emits a luminescent radiation at room temperature when a voltage is applied across the device; wherein the luminescent first radiation component contributed from the acceptor compound with an emission λₘₐₓ₁ being independently selected from the group consisting of larger than 340 nm to equal or less than 500 nm, larger than 500 nm to equal or less than 600 nm, and larger than 600 nm to equal or less than 900 nm. In some embodiments, the first radiation component has FWHM of 50, 40, 35, 30, 25, 20, 15, 10, or 5 nm or less. In some embodiments, the first radiation component has a 10% onset of the emission peak is less than 465, 460, 455, or 450 nm.

In some embodiments, the sensitizer compound is partially or fully deuterated. In some embodiments, the acceptor compound is partially or fully deuterated. In some embodiments, the first host is partially or fully deuterated. In some embodiments, the second host is partially or fully deuterated.

In some embodiments, the sensitizer compound and/or the acceptor compound each independently comprises at least one substituent having a spherocity greater than or equal to 0.45, 0.55, 0.65, 0.75, or 0.80. The spherocity is a measurement of the three-dimensionality of bulky groups. Spherocity is defined as the ratio between the principal moments of inertia (PMI). Specifically, spherocity is the ratio of three times PMI1 over the sum of PMI1, PMI2, and PMI3, where PMI1 is the smallest principal moment of inertia, PMI2 is the second smallest principal moment of inertia, and PMI3 is the largest principal moment of inertia. The spherocity of the lowest energy conformer of a structure after optimization of the ground state with density functional theory may be calculated. More detailed information can be found in paragraphs [0054] to [0059] of U.S. application Ser. No. 18/062,110 filed Dec. 6, 2022, the contents of which are incorporated herein by reference. In some embodiments, the sensitizer compound and/or the acceptor compound each independently comprises at least one substituent having a Van der Waals volume greater than 153, 206, 259, 290, or 329 Å³. In some embodiments, compound S1 and/or compound A1 each independently comprises at least one substituent having a molecular weight greater than 167, 187, 259, 303, or 305 amu.

In some embodiments, one of the first and second hosts is a hole transporting host, the other one of the first and second host is an electron transporting host. In some embodiments, the first host is a hole transporting host; and wherein the first host comprises at least one chemical group selected from the group consisting of amino, carbazole, indolocarbazole, dibenzothiophene, dibenzofuran, dibenzoselenophene, and 5λ²-benzo[d]benzo[4,5]imidazo[3,2-a]imidazole. In some embodiments, the first host is an electron transporting host; and wherein the first host comprises at least one chemical group selected from the group consisting of pyridine, pyrimidine, pyrazine, pyridazine, triazine, imidazole, aza-triphenylene, aza-carbazole, aza-dibenzothiophene, aza-dibenzofuran, aza-dibenzoselenophene, 5,9-dioxa-13b-boranaphtho[3,2,1-de]anthracene, boryl, nitrile, aza-5λ²-benzo[d]benzo[4,5]imidazo[3,2-a]imidazole, and aza-(5,9-dioxa-13b-boranaphtho[3,2,1-de]anthracene). In some embodiments, one of the first and second hosts is a bipolar host comprising both hole transporting and electron transporting moieties.

In some embodiments, the OLED further comprises a color conversion layer or a color filter.

In some embodiments, a formulation can comprise at least two different compounds of the following compounds: a sensitizer compound, an acceptor compound and a host.

In some embodiments, a chemical structure selected from the group consisting of a monomer, a polymer, a macromolecule, and a supramolecule, wherein the chemical structure comprises at least two of the following components: a sensitizer compound, an acceptor compound and a host.

In some embodiments, a premixed co-evaporation source that is a mixture of a first compound and a second compound; wherein the co-evaporation source is a co-evaporation source for vacuum deposition process or OVJP process; wherein the first compound and the second compound are differently selected from the group 1 consisting of: a sensitizer compound, an acceptor compound, a first host compound; and a second host compound; wherein the first compound has an evaporation temperature T1 of 150 to 350° C.; wherein the second compound has an evaporation temperature T2 of 150 to 350° C.; wherein absolute value of T1−T2 is less than 20° C.; wherein the first compound has a concentration C1 in said mixture and a concentration C2 in a test film formed by evaporating the mixture in a vacuum deposition tool at a constant pressure between 1×10⁻⁶ Torr to 1×10⁻⁹ Torr, at a 2 Å/sec deposition rate on a surface positioned at a predefined distance away from the mixture being evaporated; and wherein the absolute value of (C1-C2)/C1 is less than 5%. In some embodiments, the mixture further comprises a third compound; wherein the third compound is different from the first and the second compound, and is selected from the same group 1; wherein the third compound has an evaporation temperature T3 of 150 to 350° C., and wherein absolute value of T1−T3 is less than 20° C.

In some embodiments, the first compound has evaporation temperature T1 of 200 to 350° C. and the second compound has evaporation temperature T2 of 200 to 350° C. In some embodiments, the absolute value of (C₁−C₂)/C₁ is less than 3%. In some embodiments, the first compound has a vapor pressure of P₁ at T1 at 1 atm, and the second compound has a vapor pressure of P₂ at T2 at 1 atm; and wherein the ratio of P₁/P₂ is within the range of 0.90:1 to 1.10:1. In some embodiments, the first compound has a first mass loss rate and the second compound has a second mass loss rate, wherein the ratio between the first mass loss rate and the second mass loss rate is within the range of 0.90:1 to 1.10:1, 0.95:1 to 1.05:1, or 0.97:1 to 1.03:1. In some embodiments, the first compound and the second compound each has a purity in excess of 99% as determined by high pressure liquid chromatography. In some embodiments, the composition is in liquid form at a temperature less than the lesser of T1 and T2.

In some embodiments, a method for fabricating an organic light emitting device can comprises: providing a substrate having a first electrode disposed thereon; depositing a first organic layer over the first electrode by evaporating a pre-mixed co-evaporation source that is a mixture of a first compound and a second compound described above in a high vacuum deposition tool with a chamber base pressure between 1×10⁻⁶ Torr to 1×10⁻⁹ Torr; and depositing a second electrode over the first organic layer.

It should be understood that embodiments of all the compounds and devices described herein may be interchangeable if those embodiments are also applicable under different aspects of the entire disclosure.

In some embodiments, each of the sensitizer compound, the acceptor compound, the host compound, [Add the second acceptor compound A2; and the second host compound H2 if appropriate.] described herein can be at least 10% deuterated, at least 20% deuterated, at least 30% deuterated, at least 40% deuterated, at least 50% deuterated, at least 60% deuterated, at least 70% deuterated, at least 80% deuterated, at least 90% deuterated, at least 95% deuterated, at least 99% deuterated, or 100% deuterated. As used herein, percent deuteration has its ordinary meaning and includes the percent of possible hydrogen atoms (e.g., positions that are hydrogen or deuterium) that are replaced by deuterium atoms.

### D. Other Aspects of the OLEDs of the Present Disclosure

In some embodiments, the OLED may further comprise an additional host, wherein the additional host comprises a triphenylene containing benzo-fused thiophene or benzo-fused furan, wherein any substituent in the host is an unfused substituent independently selected from the group consisting of CₙH₂ₙ₊₁, OCₙH₂ₙ₊₁, OAr₁, N(CₙH₂ₙ₊₁)₂, N(Ar₁)(Ar₂), CH═CH—CₙH₂ₙ₊₁, C≡CCₙH₂ₙ₊₁, Ar₁, Ar₁—Ar₂, CₙH₂ₙ—Ar₁, or no substitution, wherein n is an integer from 1 to 10; and wherein Ar₁ and Ar₂ are independently selected from the group consisting of benzene, biphenyl, naphthalene, triphenylene, carbazole, and heteroaromatic analogs thereof.

In some embodiments, the additional host comprises at least one chemical group selected from the group consisting of triphenylene, carbazole, indolocarbazole, dibenzothiophene, dibenzofuran, dibenzoselenophene, 5′²-benzo[d]benzo[4,5]imidazo[3,2-a]imidazole, 5,9-dioxa-13b-boranaphtho[3,2,1-de]anthracene, azaborinine, oxaborinine, dihydroacridine, xanthene, dihydrobenzoazasiline, dibenzooxasiline, phenoxazine, phenoxathiine, phenothiazine, dihydrophenazine, fluorene, naphthalene, anthracene, phenanthrene, phenanthroline, benzoquinoline, quinoline, isoquinoline, quinazoline, pyrimidine, pyrazine, pyridine, triazine, boryl, silyl, aza-triphenylene, aza-carbazole, aza-indolocarbazole, aza-dibenzothiophene, aza-dibenzofuran, aza-dibenzoselenophene, aza-5λ²-benzo[d]benzo[4,5]imidazo[3,2-a]imidazole, and aza-(5,9-dioxa-13b-boranaphtho[3,2,1-de]anthracene).

In some embodiments, the additional host comprises a metal complex.

In some embodiments, the additional host is selected from the group consisting of EG1-MG1-EG1 to EG53-MG27-EG53 with a formula of EGa-MGb-EGc, or EG1-EG1 to EG53-EG53 with a formula of EGa-EGc when MGb is absent, wherein a is an integer from 1 to 53, b is an integer from 1 to 27, c is an integer from 1 to 53. The structure of EG1 to EG53 is shown below:

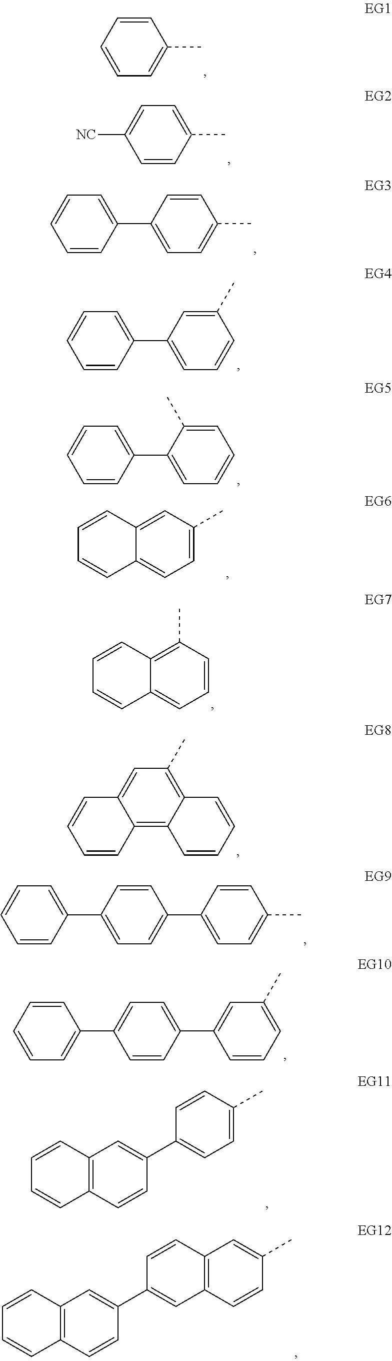

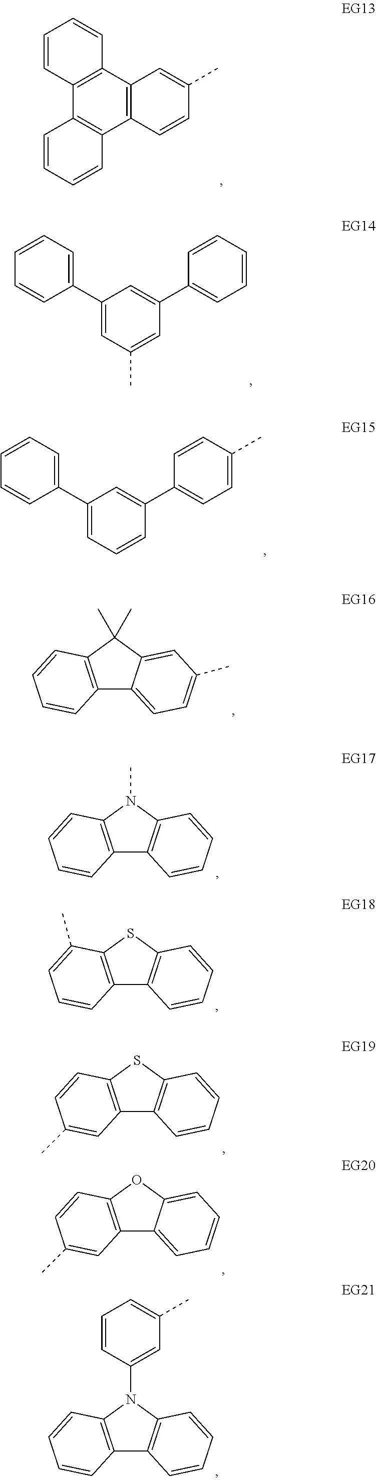

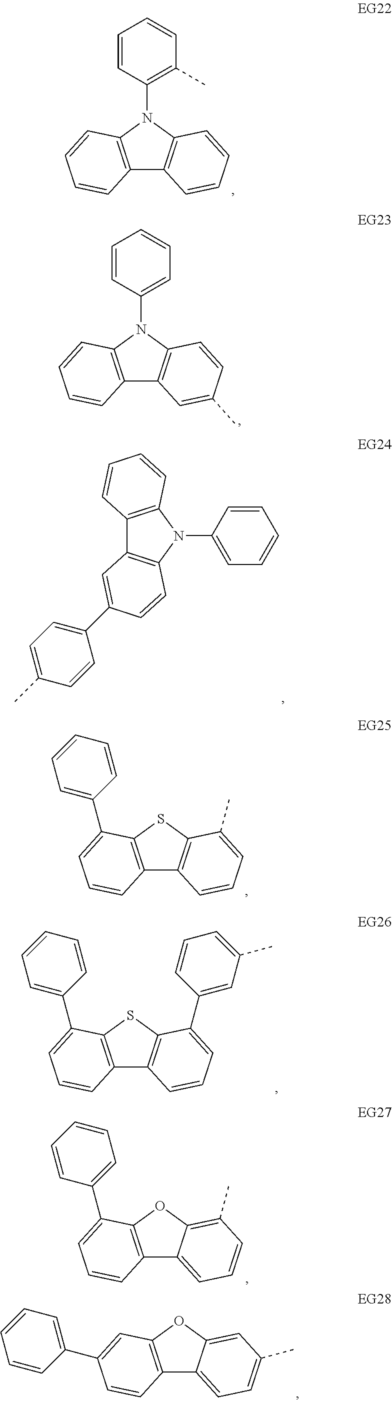

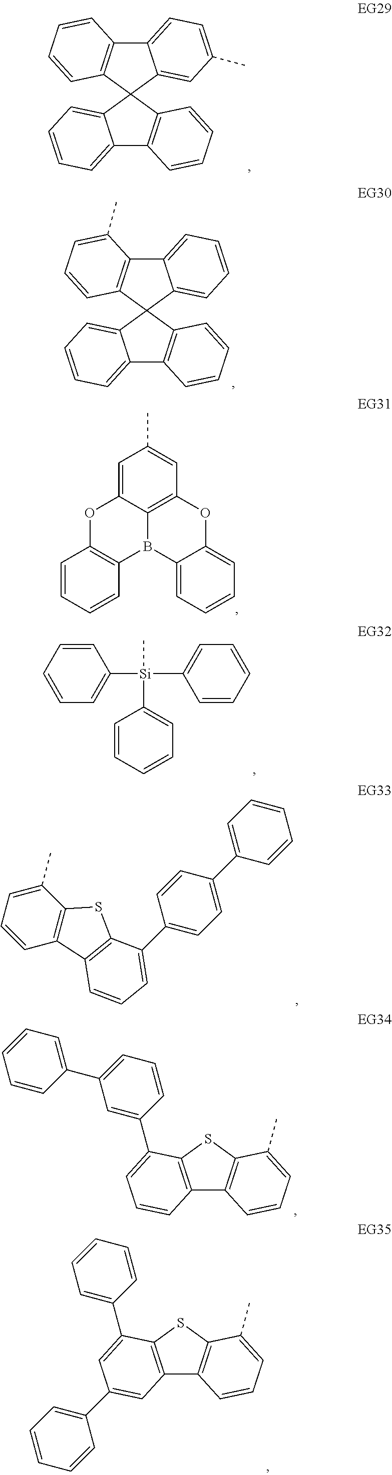

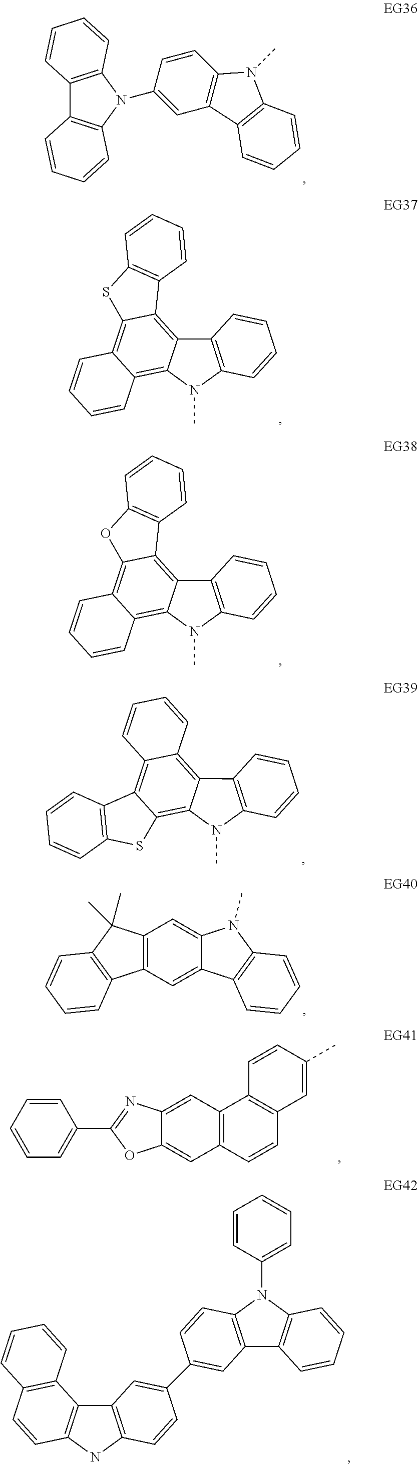

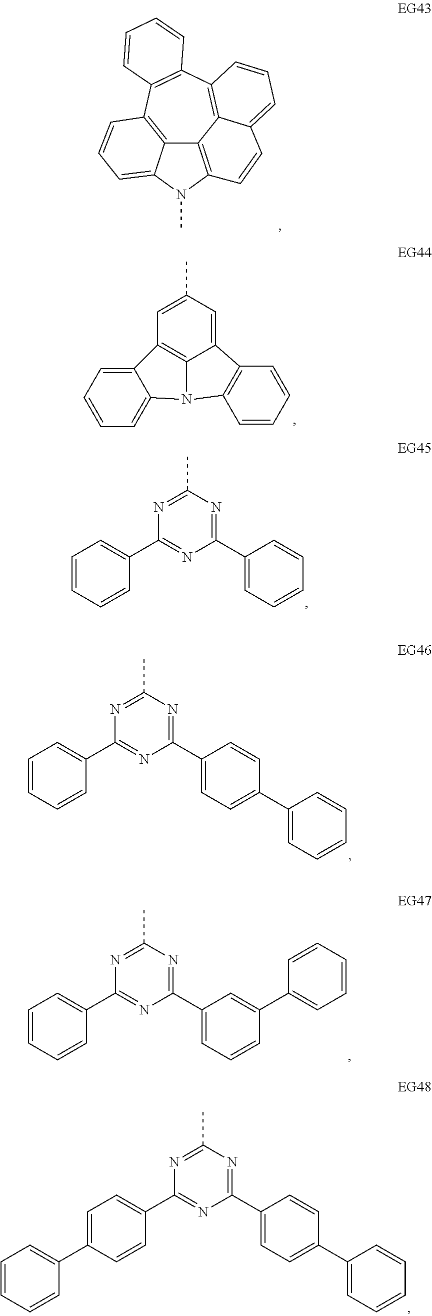

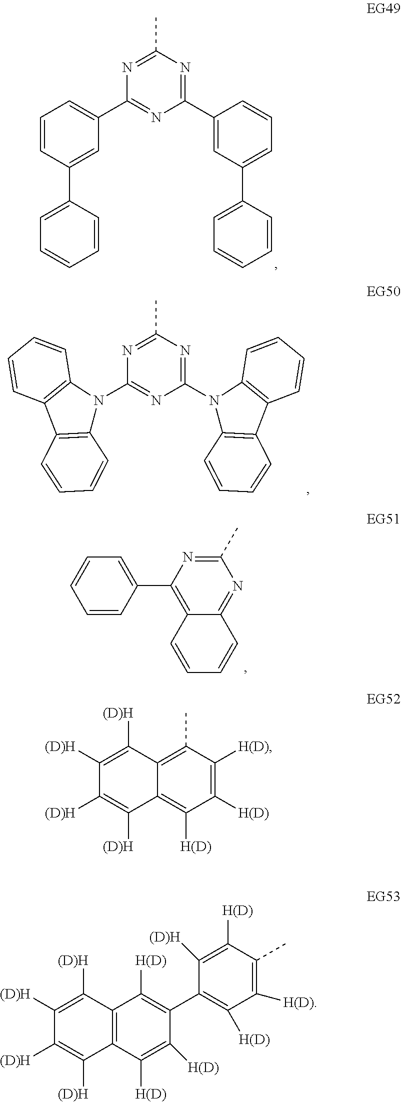

The structure of MG1 to MG27 is shown below:

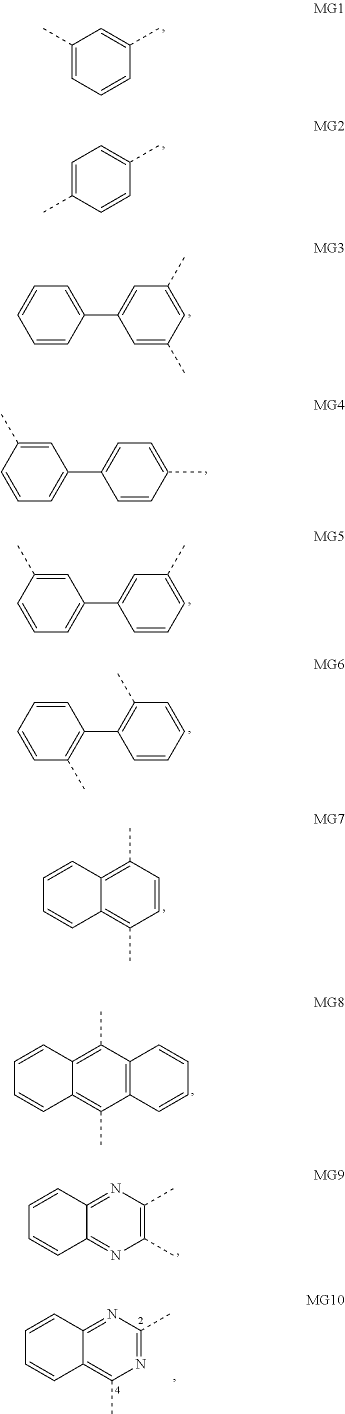

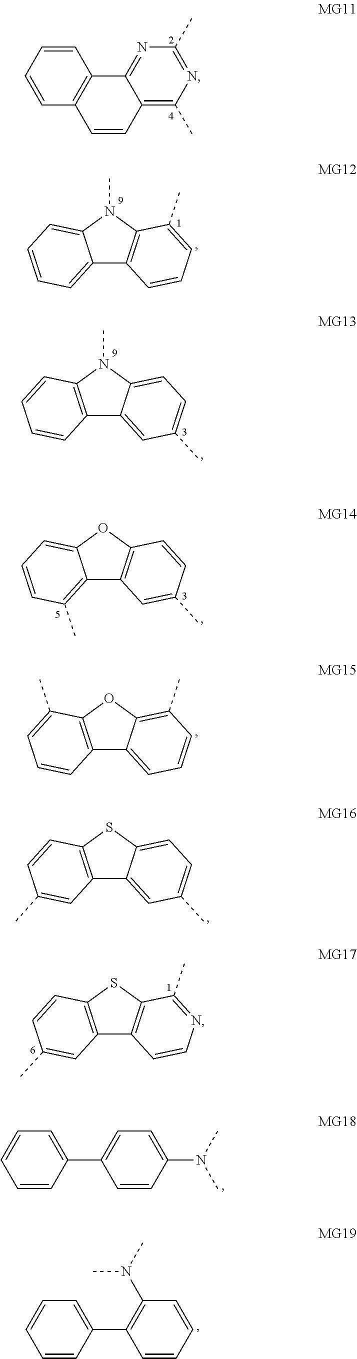

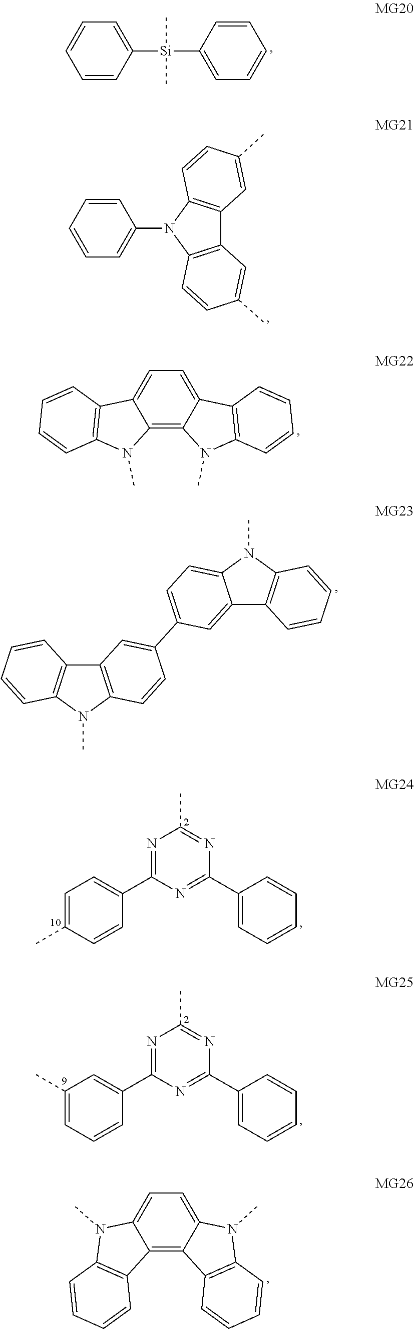

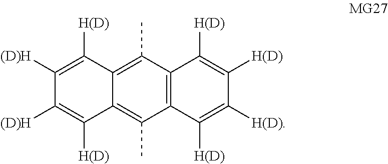

In the MGb structures shown above, the two bonding positions in the asymmetric structures MG10, MG11, MG12, MG13, MG14, MG17, MG24, and MG25 are labeled with numbers for identification purposes.

In some embodiments, the host can be any of the aza-substituted variants thereof, fully or partially deuterated variants thereof, and combinations thereof. In some embodiments, the host has formula EGa-MGb-Egc and is selected from the group consisting of h1 to h112 defined in the following HOST Group 2 list, where each of MGb, EGa, and EGc are defined as follows:

| h | MGb | EGa | EGc |
|---|---|---|---|
| h1 | MG1 | EG3 | EG36 |
| h2 | MG1 | EG8 | EG12 |
| h3 | MG1 | EG13 | EG14 |
| h4 | MG1 | EG13 | EG18 |
| h5 | MG1 | EG13 | EG25 |
| h6 | MG1 | EG13 | EG36 |
| h7 | MG1 | EG22 | EG36 |
| h8 | MG1 | EG25 | EG46 |
| h9 | MG1 | EG27 | EG46 |
| h10 | MG1 | EG27 | EG48 |
| h11 | MG1 | EG32 | EG50 |
| h12 | MG1 | EG35 | EG46 |
| h13 | MG1 | EG36 | EG45 |
| h14 | MG1 | EG36 | EG49 |
| h15 | MG1 | EG40 | EG45 |
| h16 | MG2 | EG3 | EG36 |
| h17 | MG2 | EG25 | EG31 |
| h18 | MG2 | EG31 | EG33 |
| h19 | MG2 | EG36 | EG45 |
| h20 | MG2 | EG36 | EG46 |
| h21 | MG3 | EG4 | EG36 |
| h22 | MG3 | EG34 | EG45 |
| h23 | MG4 | EG13 | EG17 |
| h24 | MG5 | EG13 | EG45 |
| h25 | MG5 | EG17 | EG36 |
| h26 | MG5 | EG18 | EG36 |
| h27 | MG6 | EG17 | EG17 |
| h28 | MG7 | EG43 | EG45 |
| h29 | MG8 | EG1 | EG28 |
| h30 | MG8 | EG6 | EG7 |
| h31 | MG8 | EG7 | EG7 |
| h32 | MG8 | EG7 | EG11 |
| h33 | MG9 | EG1 | EG43 |
| h34 | MG10 | 4-EG1 | 2-EG37 |
| h35 | MG10 | 4-EG1 | 2-EG38 |
| h36 | MG10 | EG1 | EG42 |
| h37 | MG11 | 4-EG1 | 2-EG39 |
| h38 | MG12 | 1-EG17 | 9-EG31 |
| h39 | MG13 | 3-EG17 | 9-EG4 |
| h40 | MG13 | 3-EG17 | 9-EG13 |
| h41 | MG13 | 3-EG17 | 9-EG31 |
| h42 | MG13 | 3-EG17 | 9-EG45 |
| h43 | MG13 | 3-EG17 | 9-EG46 |
| h44 | MG13 | 3-EG17 | 9-EG48 |
| h45 | MG13 | 3-EG17 | 9-EG49 |
| h46 | MG13 | 3-EG32 | 9-EG31 |
| h47 | MG13 | 3-EG44 | 9-EG3 |
| h48 | MG14 | 3-EG13 | 5-EG45 |
| h49 | MG14 | 3-EG23 | 5-EG45 |
| h50 | MG15 | EG3 | EG48 |
| h51 | MG15 | EG17 | EG31 |
| h52 | MG15 | EG31 | EG36 |
| h53 | MG16 | EG17 | EG17 |
| h54 | MG17 | EG17 | EG17 |
| h55 | MG18 | EG16 | EG24 |
| h56 | MG18 | EG16 | EG30 |
| h57 | MG18 | EG20 | EG41 |
| h58 | MG19 | EG16 | EG29 |
| h59 | MG20 | EG1 | EG31 |
| h60 | MG20 | EG17 | EG18 |
| h61 | MG21 | EG23 | EG23 |
| h62 | MG22 | EG1 | EG45 |
| h63 | MG22 | EG1 | EG46 |
| h64 | MG22 | EG3 | EG46 |
| h65 | MG22 | EG4 | EG46 |
| h66 | MG22 | EG4 | EG47 |
| h67 | MG22 | EG9 | EG45 |
| h68 | MG23 | EG1 | EG3 |
| h69 | MG23 | EG1 | EG6 |
| h70 | MG23 | EG1 | EG14 |
| h71 | MG23 | EG1 | EG18 |
| h72 | MG23 | EG1 | EG19 |
| h73 | MG23 | EG1 | EG23 |
| h74 | MG23 | EG1 | EG51 |
| h75 | MG23 | EG2 | EG18 |
| h76 | MG23 | EG3 | EG3 |
| h77 | MG23 | EG3 | EG4 |
| h78 | MG23 | EG3 | EG5 |
| h79 | MG23 | EG4 | EG4 |
| h80 | MG23 | EG4 | EG5 |
| h81 | MG24 | 2-EG1 | 10-EG33 |
| h82 | MG24 | 2-EG4 | 10-EG36 |
| h83 | MG24 | 2-EG21 | 10-EG36 |
| h84 | MG24 | 2-EG23 | 10-EG36 |
| h85 | MG25 | 2-EG1 | 9-EG33 |
| h86 | MG25 | 2-EG3 | 9-EG36 |
| h87 | MG25 | 2-EG4 | 9-EG36 |
| h88 | MG25 | 2-EG17 | 9-EG27 |
| h89 | MG25 | 2-EG17 | 9-EG36 |
| h90 | MG25 | 2-EG21 | 9-EG36 |
| h91 | MG25 | 2-EG23 | 9-EG27 |
| h92 | MG25 | 2-EG23 | 9-EG36 |
| h93 | MG26 | EG1 | EG9 |
| h94 | MG26 | EG1 | EG10 |
| h95 | MG26 | EG1 | EG21 |
| h96 | MG26 | EG1 | EG23 |
| h97 | MG26 | EG1 | EG26 |
| h98 | MG26 | EG3 | EG3 |
| h99 | MG26 | EG3 | EG9 |
| h100 | MG26 | EG3 | EG23 |
| h101 | MG26 | EG3 | EG26 |
| h102 | MG26 | EG4 | EG10 |
| h103 | MG26 | EG5 | EG10 |
| h104 | MG26 | EG6 | EG10 |
| h105 | MG26 | EG10 | EG10 |
| h106 | MG26 | EG10 | EG14 |
| h107 | MG26 | EG10 | EG15 |
| h108 | MG27 | EG52 | EG53 |
| h109 | — | EG13 | EG18 |
| h110 | — | EG17 | EG31 |
| h111 | — | EG17 | EG50 |
| h112 | — | EG40 | EG45 |

In the table above, the EGa and EGc structures that are bonded to one of the asymmetric structures MG10, MG11, MG12, MG13, MG14, MG17, MG24, and MG25, are noted with a numeric prefix identifying their bonding position in the MGb structure.

In yet another aspect, the OLED of the present disclosure may also comprise an emissive region containing a formulation as disclosed in the above compounds section of the present disclosure.

In some embodiments, the emissive region consists of one or more organic layers, wherein at least one of the one or more organic layers has a minimum thickness selected from the group consisting of 350, 400, 450, 500, 550, 600, 650 and 700 Å. In some embodiments, the at least one of the one or more organic layers are formed from an Emissive System that has a figure of merit (FOM) value equal to or larger than the number selected from the group consisting of 2.50, 2.55, 2.60, 2.65, 2.70, 2.75, 2.80, 2.85, 2.90, 2.95, 3.00, 5.00, 10.0, 15.0, and 20.0. The definition of FOM is available in U.S. patent Application Publication No. 2023/0292605, and its entire contents are incorporated herein by reference. In some embodiments, the at least one of the one or more organic layers comprises a compound or a formulation of the compound as disclosed in Sections A and D of the present disclosure.

In some embodiments, the OLED or the emissive region disclosed herein can be incorporated into a full-color pixel arrangement of a device. The full-color pixel arrangement of such device comprises at least one pixel, wherein the at least one pixel comprises a first subpixel and a second subpixel. The first subpixel includes a first OLED comprising a first emissive region. The second subpixel includes a second OLED comprising a second emissive region. In some embodiments, the first and/or second OLED, the first and/or second emissive region can be the same or different and each can independently have the various device characteristics and the various embodiments of the inventive compounds included therein, and various combinations and subcombinations of the various device characteristics and the various embodiments of the inventive compounds included therein, as disclosed herein.

In some embodiments, the first emissive region is configured to emit a light having a peak wavelength λₘₐₓ₁; the second emissive region is configured to emit a light having a peak wavelength λₘₐₓ₂. In some embodiments, the difference between the peak wavelengths λₘₐₓ₁ and λₘₐₓ₂ is at least 4 nm but within the same color. For example, a light blue and a deep blue light as described above. In some embodiments, a first emissive region is configured to emit a light having a peak wavelength λₘₐₓ₁ in one region of the visible spectrum of 400-500 nm, 500-600 nm, 600-700 nm; and a second emissive region is configured to emit light having a peak wavelength λₘₐₓ₂ in one of the remaining regions of the visible spectrum of 400-500 nm, 500-600 nm, 600-700 nm. In some embodiments, the first emissive region comprises a first number of emissive layers that are deposited one over the other if more than one; and the second emissive region comprises a second number of emissive layers that is deposited one over the other if more than one; and the first number is different from the second number. In some embodiments, both the first emissive region and the second emissive region comprise a phosphorescent materials, which may be the same or different. In some embodiments, the first emissive region comprises a phosphorescent material, while the second emissive region comprises a fluorescent material. In some embodiments, both the first emissive region and the second emissive region comprise a fluorescent materials, which may be the same or different.

In some embodiments, the at least one pixel of the OLED or emissive regions includes a total of N subpixels;
wherein the N subpixels comprise the first subpixel and the second subpixel; wherein each of the N subpixels comprises an emissive region; wherein the total number of the emissive regions within the at least one pixel is equal to or less than N−1. In some embodiments, the second emissive region is exactly the same as the first emissive region; and each subpixel of the at least one pixel comprises the same one emissive region as the first emissive region. In some embodiments, the full-color pixel arrangements can have a plurality of pixels comprising a first pixel region and a second pixel region; wherein at least one display characteristic in the first pixel region is different from the corresponding display characteristic of the second pixel region, and wherein the at least one display characteristic is selected from the group consisting of resolution, cavity mode, color, outcoupling, and color filter.

In some embodiments, the OLED is a stacked OLED comprising one or more charge generation layers (CGLs). In some embodiments, the OLED comprises a first electrode, a first emissive region disposed over the first electrode, a first CGL disposed over the first emissive region, a second emissive region disposed over the first CGL, and a second electrode disposed over the second emissive region. In some embodiments, the first and/or the second emissive regions can have the various device characteristics as described above for the pixelated device. In some embodiments, the stacked OLED is configured to emit white color. In some embodiments, one or more of the emissive regions in a pixelated or in a stacked OLED comprises a sensitizer and an acceptor with the various sensitizing device characteristics and the various embodiments of the inventive compounds disclosed herein. For example, the first emissive region is comprised in a sensitizing device, while the second emissive region is not comprised in a sensitizing device; in some instances, both the first and the second emissive regions are comprised in sensitizing devices.

In some embodiments, the OLED can emit light having at least 1%, 5%, 10, 30%, 50%, 70%, 80%, 90%, 95%, 99%, or 100% from the plasmonic mode. In some embodiments, at least one of the anode, the cathode, or a new layer disposed next to the anode or cathode functions as an enhancement layer. The enhancement layer comprises a plasmonic material exhibiting surface plasmon resonance that non-radiatively couples to the emitter material and transfers excited state energy from the emitter material to non-radiative mode of surface plasmon polariton. In some embodiments, the enhancement layer is provided no more than a threshold distance away from the organic emissive layer, wherein the emitter material has a total non-radiative decay rate constant and a total radiative decay rate constant due to the presence of the enhancement layer. A threshold distance is where the total non-radiative decay rate constant is equal to the total radiative decay rate constant. Another threshold distance is the distance at which the total radiative decay rate constant divided by the sum of the total non-radiative decay rate constant and total radiative decay rate constant is equal to the photoluminescent yield of the emissive material without the enhancement layer present.

In some embodiments, the OLED further comprises an outcoupling layer. In some embodiments, the outcoupling layer is disposed over the enhancement layer on a side opposite the organic emissive layer The outcoupling layer scatters the energy from the surface plasmon polaritons. In some embodiments this energy is scattered as photons to free space. In other embodiments, the energy is scattered from the surface plasmon mode into other modes of the device such as but not limited to the organic waveguide mode, the substrate mode, or another waveguiding mode. In some embodiments, one or more intervening layer can be disposed between the enhancement layer and the outcoupling layer. The examples for intervening layer(s) can be dielectric materials, including organic, inorganic, perovskites, oxides, and may include stacks and/or mixtures of these materials.

The enhancement layer modifies the effective properties of the medium in which the emitter material resides resulting in any or all of the following: a decreased rate of emission, a modification of emission line-shape, a change in emission intensity with angle, a change in the stability of the emitter material, a change in the efficiency of the OLED, and a reduced efficiency roll-off of the OLED device. Placement of the enhancement layer on the cathode side, anode side, or on both sides, or the enhancement layer itself being as the CGL, results in OLED devices which take advantage of any of the above-mentioned effects. In addition to the specific functional layers mentioned herein and illustrated in the various OLED examples shown in the figures, the OLEDs according to the present disclosure may include any of the other functional layers often found in OLEDs.

In some embodiments, the enhancement layer can be comprised of plasmonic materials, optically active metamaterials, or hyperbolic metamaterials. In some embodiments, the plasmonic material includes at least one metal. In such embodiments the metal may include at least one of Ag, Al, Au, Ir, Pt, Ni, Cu, W, Ta, Fe, Cr, Mg, Ga, Rh, Ti, Ru, Pd, In, Bi, or Ca, alloys or mixtures of these materials, and stacks of these materials. In some embodiments, the enhancement layer is provided as a planar layer. In other embodiments, the enhancement layer has wavelength-sized features that are arranged periodically, quasi-periodically, or randomly, or sub-wavelength-sized features that are arranged periodically, quasi-periodically, or randomly.

In some embodiments, the outcoupling layer has wavelength-sized or sub-wavelength sized features that are arranged periodically, quasi-periodically, or randomly. In some embodiments, the outcoupling layer may be composed of a plurality of nanoparticles. In some embodiments, the outcoupling layer is composed of a plurality of nanoparticles disposed over a material. In these embodiments the outcoupling layer may be tunable by at least one of: varying a size of the plurality of nanoparticles, varying a shape of the plurality of nanoparticles, changing a material of the plurality of nanoparticles, adjusting a thickness of the material, changing the refractive index of the material, adding an additional layer disposed on the plurality of nanoparticles, varying a thickness of the enhancement layer, or varying the material of the enhancement layer. The plurality of nanoparticles of the device may be formed from at least one of metal, dielectric material, semiconductor materials, an alloy of metal, a mixture of dielectric materials, a stack or layering of one or more materials, and/or a core of one type of material and that is coated with a shell of a different type of material. In some embodiments, the outcoupling layer is composed of at least metal nanoparticles wherein the metal is selected from the group consisting of Ag, Al, Au, Ir, Pt, Ni, Cu, W, Ta, Fe, Cr, Mg, Ga, Rh, Ti, Ru, Pd, In, Bi, and Ca, alloys or mixtures of these materials, and stacks of these materials. In some embodiments the outcoupling layer is formed by lithography. In some embodiments, the outcoupling layer is composed of at least dielectric nanoparticles wherein the dielectric material is selected from the group consisting of silicon, silicon nitride, boron nitride, silicon carbide, carbon, diamond, zinc sulfide, zinc selenide, germanium, zinc telluride, potassium niobate, titanium oxide, antimony oxide, niobium pentoxide, tantalum pentoxide, vanadium oxide, vanadium pentoxide, gallium phosphate, bismuth oxide, gallium arsenide, indium oxide, silicon dioxide, aluminum gallium, molybdenum oxide, alloys or mixtures of these materials, and stacks of these materials.

In some embodiments of a plasmonic device, the emitter, and/or host compounds used in the emissive layer has a vertical dipole ratio (VDR) of 0.33 or more. In some such embodiments, the emitter, and/or host compounds have a VDR of 0.40, 0.50, 0.60, 0.70, or more.

In yet another aspect, the present disclosure also provides a consumer product comprising an organic light-emitting device (OLED) having an anode; a cathode; and an organic layer disposed between the anode and the cathode, wherein the organic layer may comprise a compound as disclosed in the above compounds section of the present disclosure.

In some embodiments, the consumer product comprises an OLED having an anode; a cathode; and an organic layer disposed between the anode and the cathode, wherein the organic layer may comprise a formulation as described herein.

In some embodiments, the consumer product can be one of a flat panel display, a computer monitor, a medical monitor, a television, a billboard, a light for interior or exterior illumination and/or signaling, a heads-up display, a fully or partially transparent display, a flexible display, a laser printer, a telephone, a cell phone, tablet, a phablet, a personal digital assistant (PDA), a wearable device, a laptop computer, a digital camera, a camcorder, a viewfinder, a micro-display that is less than 2 inches diagonal, a 3-D display, a virtual reality or augmented reality display, a vehicle, a video wall comprising multiple displays tiled together, a theater or stadium screen, a light therapy device, and a sign.

Generally, an OLED comprises at least one organic layer disposed between and electrically connected to an anode and a cathode. When a current is applied, the anode injects holes and the cathode injects electrons into the organic layer(s). The injected holes and electrons each migrate toward the oppositely charged electrode. When an electron and hole localize on the same molecule, an “exciton,” which is a localized electron-hole pair having an excited energy state, is formed. Light is emitted when the exciton relaxes via a photoemissive mechanism. In some cases, the exciton may be localized on an excimer or an exciplex. Non-radiative mechanisms, such as thermal relaxation, may also occur, but are generally considered undesirable.

Several OLED materials and configurations are described in U.S. Pat. Nos. 5,844,363, 6,303,238, and 5,707,745, which are incorporated herein by reference in their entirety.

The initial OLEDs used emissive molecules that emitted light from their singlet states (“fluorescence”) as disclosed, for example, in U.S. Pat. No. 4,769,292, which is incorporated by reference in its entirety. Fluorescent emission generally occurs in a time frame of less than 10 nanoseconds.

More recently, OLEDs having emissive materials that emit light from triplet states (“phosphorescence”) have been demonstrated. Baldo et al., “Highly Efficient Phosphorescent Emission from Organic Electroluminescent Devices,” Nature, vol. 395, 151-154, 1998; (“Baldo-I”) and Baldo et al., “Very high-efficiency green organic light-emitting devices based on electrophosphorescence,” Appl. Phys. Lett., vol. 75, No. 3, 4-6 (1999) (“Baldo-II”), are incorporated by reference in their entireties. Phosphorescence is described in more detail in U.S. Pat. No. 7,279,704 at cols. 5-6, which are incorporated by reference.

FIG. 1 shows an organic light emitting device 100. The figures are not necessarily drawn to scale. Device 100 may include a substrate 110, an anode 115, a hole injection layer 120, a hole transport layer 125, an electron blocking layer 130, an emissive layer 135, a hole blocking layer 140, an electron transport layer 145, an electron injection layer 150, a protective layer 155, a cathode 160, and a barrier layer 170. Cathode 160 is a compound cathode having a first conductive layer 162 and a second conductive layer 164. Device 100 may be fabricated by depositing the layers described, in order. The properties and functions of these various layers, as well as example materials, are described in more detail in U.S. Pat. No. 7,279,704 at cols. 6-10, which are incorporated by reference.

More examples for each of these layers are available. For example, a flexible and transparent substrate-anode combination is disclosed in U.S. Pat. No. 5,844,363, which is incorporated by reference in its entirety. An example of a p-doped hole transport layer is m-MTDATA doped with F₄-TCNQ at a molar ratio of 50:1, as disclosed in U.S. Patent Application Publication No. 2003/0230980, which is incorporated by reference in its entirety. Examples of emissive and host materials are disclosed in U.S. Pat. No. 6,303,238 to Thompson et al., which is incorporated by reference in its entirety. An example of an n-doped electron transport layer is BPhen doped with Li at a molar ratio of 1:1, as disclosed in U.S. Patent Application Publication No. 2003/0230980, which is incorporated by reference in its entirety. U.S. Pat. Nos. 5,703,436 and 5,707,745, which are incorporated by reference in their entireties, disclose examples of cathodes including compound cathodes having a thin layer of metal such as Mg:Ag with an overlying transparent, electrically-conductive, sputter-deposited ITO layer. The theory and use of blocking layers is described in more detail in U.S. Pat. No. 6,097,147 and U.S. Patent Application Publication No. 2003/0230980, which are incorporated by reference in their entireties. Examples of injection layers are provided in U.S. Patent Application Publication No. 2004/0174116, which is incorporated by reference in its entirety. A description of protective layers may be found in U.S. Patent Application Publication No. 2004/0174116, which is incorporated by reference in its entirety.

FIG. 2 shows an inverted OLED 200. The device includes a substrate 210, a cathode 215, an emissive layer 220, a hole transport layer 225, and an anode 230. Device 200 may be fabricated by depositing the layers described, in order. Because the most common OLED configuration has a cathode disposed over the anode, and device 200 has cathode 215 disposed under anode 230, device 200 may be referred to as an “inverted” OLED. Materials similar to those described with respect to device 100 may be used in the corresponding layers of device 200. FIG. 2 provides one example of how some layers may be omitted from the structure of device 100.

The simple layered structure illustrated in FIGS. 1 and 2 is provided by way of non-limiting example, and it is understood that embodiments of the present disclosure may be used in connection with a wide variety of other structures. The specific materials and structures described are exemplary in nature, and other materials and structures may be used. Functional OLEDs may be achieved by combining the various layers described in different ways, or layers may be omitted entirely, based on design, performance, and cost factors. Other layers not specifically described may also be included. Materials other than those specifically described may be used. Although many of the examples provided herein describe various layers as comprising a single material, it is understood that combinations of materials, such as a mixture of host and dopant, or more generally a mixture, may be used. Also, the layers may have various sublayers. The names given to the various layers herein are not intended to be strictly limiting. For example, in device 200, hole transport layer 225 transports holes and injects holes into emissive layer 220, and may be described as a hole transport layer or a hole injection layer. In one embodiment, an OLED may be described as having an “organic layer” disposed between a cathode and an anode. This organic layer may comprise a single layer, or may further comprise multiple layers of different organic materials as described, for example, with respect to FIGS. 1 and 2.

Structures and materials not specifically described may also be used, such as OLEDs comprised of polymeric materials (PLEDs) such as disclosed in U.S. Pat. No. 5,247,190 to Friend et al., which is incorporated by reference in its entirety. By way of further example, OLEDs having a single organic layer may be used. OLEDs may be stacked, for example as described in U.S. Pat. No. 5,707,745 to Forrest et al, which is incorporated by reference in its entirety. The OLED structure may deviate from the simple layered structure illustrated in FIGS. 1 and 2. For example, the substrate may include an angled reflective surface to improve out-coupling, such as a mesa structure as described in U.S. Pat. No. 6,091,195 to Forrest et al., and/or a pit structure as described in U.S. Pat. No. 5,834,893 to Bulovic et al., which are incorporated by reference in their entireties.

More details on OLEDs, and the definitions described above, can be found in U.S. Pat. No. 7,279,704, which is incorporated herein by reference in its entirety.

The materials and structures described herein may have applications in devices other than OLEDs. For example, other optoelectronic devices such as organic solar cells and organic photodetectors may employ the materials and structures. More generally, organic devices, such as organic transistors, may employ the materials and structures.

In some embodiments, the OLED has one or more characteristics selected from the group consisting of being flexible, being rollable, being foldable, being stretchable, and being curved. In some embodiments, the OLED is transparent or semi-transparent. In some embodiments, the OLED further comprises a layer comprising carbon nanotubes. In some embodiments, the OLED further comprises one or more quantum dots. Such quantum dots can be in the emissive layer, or in other functional layers, such as a down conversion layer.

In some embodiments, the OLED further comprises a layer comprising a delayed fluorescent emitter. In some embodiments, the OLED comprises a RGB pixel arrangement or white plus color filter pixel arrangement. In some embodiments, the OLED is a mobile device, a hand held device, or a wearable device. In some embodiments, the OLED is a display panel having less than 10 inch diagonal or 50 square inch area. In some embodiments, the OLED is a display panel having at least 10 inch diagonal or 50 square inch area. In some embodiments, the OLED is a lighting panel.

In some embodiments, the compound can be an emissive dopant. In some embodiments, the compound can produce emissions via phosphorescence, fluorescence, thermally activated delayed fluorescence, i.e., TADF (also referred to as E-type delayed fluorescence; see, e.g., U.S. application Ser. No. 15/700,352, which is hereby incorporated by reference in its entirety), triplet-triplet annihilation, or combinations of these processes. In some embodiments, the emissive dopant can be a racemic mixture, or can be enriched in one enantiomer. In some embodiments, the compound can be homoleptic (each ligand is the same). In some embodiments, the compound can be heteroleptic (at least one ligand is different from others). When there are more than one ligand coordinated to a metal, the ligands can all be the same in some embodiments. In some other embodiments, at least one ligand is different from the other ligands. In some embodiments, every ligand can be different from each other. This is also true in embodiments where a ligand being coordinated to a metal can be linked with other ligands being coordinated to that metal to form a tridentate, tetradentate, pentadentate, or hexadentate ligands. Thus, where the coordinating ligands are being linked together, all of the ligands can be the same in some embodiments, and at least one of the ligands being linked can be different from the other ligand(s) in some other embodiments.

In some embodiments, the compound can be used as a phosphorescent sensitizer in an OLED where one or multiple layers in the OLED contains an acceptor in the form of one or more fluorescent and/or delayed fluorescence emitters. In some embodiments, the compound can be used as one component of an exciplex to be used as a sensitizer. As a phosphorescent sensitizer, the compound must be capable of energy transfer to the acceptor and the acceptor will emit the energy or further transfer energy to a final emitter. The acceptor concentrations can range from 0.001% to 100%. The acceptor could be in either the same layer as the phosphorescent sensitizer or in one or more different layers. In some embodiments, the acceptor is a TADF emitter. In some embodiments, the acceptor is a fluorescent emitter. In some embodiments, the emission can arise from any or all of the sensitizer, acceptor, and final emitter.

According to another aspect, a formulation comprising the compounds described herein is also disclosed.

The OLED disclosed herein can be incorporated into one or more of a consumer product, an electronic component module, and a lighting panel. The organic layer can be an emissive layer and the compound can be an emissive dopant in some embodiments, while the compound can be a non-emissive dopant in other embodiments. In some embodiments, the emissive layer further optionally comprises a dopant selected from the group consisting of delayed-fluorescent, and non-delayed fluorescent.

In yet another aspect of the present disclosure, a formulation that comprises the novel compound disclosed herein is described. The formulation can include one or more components selected from the group consisting of a solvent, a host, a hole injection material, hole transport material, electron blocking material, hole blocking material, and an electron transport material, disclosed herein.

The present disclosure encompasses any chemical structure comprising the novel compound of the present disclosure, or a monovalent or polyvalent variant thereof. In other words, the inventive compound, or a monovalent or polyvalent variant thereof, can be a part of a larger chemical structure. Such chemical structure can be selected from the group consisting of a monomer, a polymer, a macromolecule, and a supramolecule (also known as supermolecule). As used herein, a “monovalent variant of a compound” refers to a moiety that is identical to the compound except that one hydrogen has been removed and replaced with a bond to the rest of the chemical structure. As used herein, a “polyvalent variant of a compound” refers to a moiety that is identical to the compound except that more than one hydrogen has been removed and replaced with a bond or bonds to the rest of the chemical structure. In the instance of a supramolecule, the inventive compound can also be incorporated into the supramolecule complex without covalent bonds.

Hence, according to one aspect, the present invention is directed to a compound, or a neutral molecular form thereof, or a monovalent or polyvalent form thereof, or a monomeric or polymeric form thereof, or a macromolecular or supramolecular form thereof; wherein the compound has a structure of the compound as described herein. In yet another aspect, the present disclosure also provides a composition of a compound of Formula I, wherein the compound of Formula I comprises its neutral molecular form, its monovalent or polyvalent form, or its monomeric or polymeric form, or its macromolecular or supramolecular form; and wherein the compound has a structure of Formula I described herein.

### E. Combination of the Compounds of the Present Disclosure with Other Materials

The materials described herein are as various examples useful for a particular layer in an OLED. They may also be used in combination with a wide variety of other materials present in the device. For example, emissive dopants disclosed herein may be used by themselves in the EML, or in conjunction with a wide variety of other emitters, hosts, transport layers, blocking layers, injection layers, electrodes and other layers that may be present. The materials described or referred to below are non-limiting examples of materials that may be useful in combination with the compounds and the devices disclosed herein, and one of ordinary skill in the art can readily consult the literature to identify other materials that may be useful in combination.

### a) Conductivity Dopants:

A charge transport layer can be doped with conductivity dopants to substantially alter its density of charge carriers, which will in turn alter its conductivity. The conductivity is increased by generating charge carriers in the matrix material, and depending on the type of dopant, a change in the Fermi level of the semiconductor may also be achieved. Hole-transporting layer can be doped by p-type conductivity dopants and n-type conductivity dopants are used in the electron-transporting layer. In some embodiments, conductivity dopants comprise at least one chemical moiety selected from the group consisting of cyano, fluorinated aryl or heteroaryl, fluorinated alkyl or cycloalkyl, alkylene, heteroaryl, amide, benzodithiophene, and highly conjugated heteroaryl groups extended by non-ring double bonds.

### b) HIL/HTL:

A hole injecting/transporting material to be used in the present disclosure is not particularly limited, and any compound may be used as long as the compound is typically used as a hole injecting/transporting material. Examples of the material include, but are not limited to: a phthalocyanine or porphyrin derivative; an aromatic amine derivative; an indolocarbazole derivative; a polymer containing fluorohydrocarbon; a polymer with conductivity dopants; a conducting polymer, such as PEDOT/PSS; a self-assembly monomer derived from compounds such as phosphonic acid and silane derivatives; a metal oxide derivative, such as MoOₓ; a p-type semiconducting organic compound, such as 1,4,5,8,9,12-Hexaazatriphenylenehexacarbonitrile; a metal complex, and a cross-linkable compounds.

Examples of aromatic amine derivatives used in HIL or HTL include, but not limit to the following general structures:

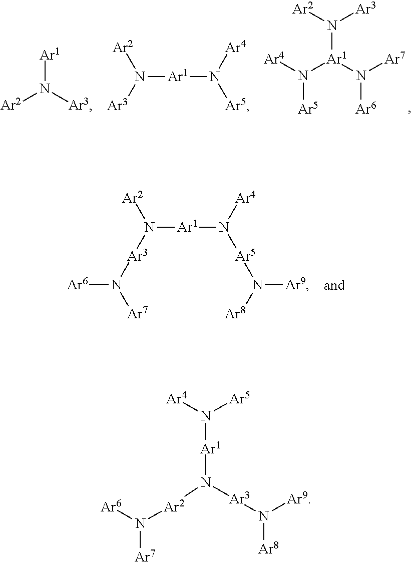

Each of Ar¹ to Ar⁹ is selected from the group consisting of aromatic hydrocarbon cyclic compounds such as benzene, biphenyl, triphenyl, triphenylene, naphthalene, anthracene, phenalene, phenanthrene, fluorene, pyrene, chrysene, perylene, and azulene; the group consisting of aromatic heterocyclic compounds such as dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine; and the group consisting of 2 to 10 cyclic structural units which are groups of the same type or different types selected from the aromatic hydrocarbon cyclic group and the aromatic heterocyclic group and are bonded to each other directly or via at least one of oxygen atom, nitrogen atom, sulfur atom, silicon atom, phosphorus atom, boron atom, chain structural unit and the aliphatic cyclic group. Each of Ar¹ to Ar⁹ may be unsubstituted or may be substituted by a general substituent as described above, any two substituents can be joined or fused into a ring.

In some embodiments, each Ar¹ to Ar⁹ independently comprises a moiety selected from the group consisting of:

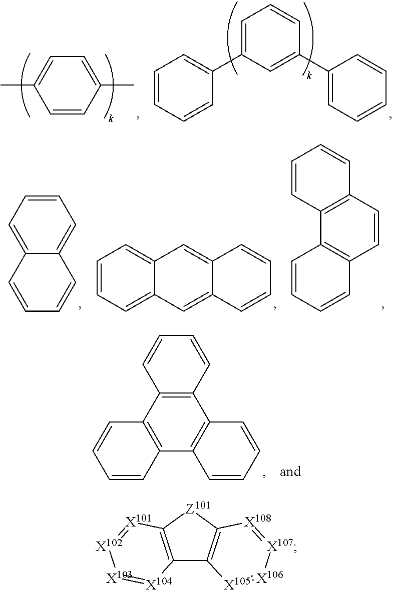

wherein k is an integer from 1 to 20; X¹⁰¹ to X¹⁰⁸ is C or N; Z¹⁰¹ is C, N, O, or S.

Examples of metal complexes used in HIL or HTL include, but are not limited to the following general formula:

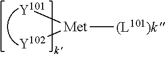

wherein Met is a metal, which can have an atomic weight greater than 40; (Y¹⁰¹—Y¹⁰²) is a bidentate ligand, the coordinating atoms of Y¹⁰¹ and Y¹⁰² are independently selected from C, N, O, P, and S; L¹⁰¹ is an another ligand; k′ is an integer value from 1 to the maximum number of ligands that may be attached to the metal; and k′+k″ is the maximum number of ligands that may be attached to the metal.

In some embodiments, (Y¹⁰¹—Y¹⁰²) is a 2-phenylpyridine or 2-phenylimidazole derivative. In some embodiments, (Y¹⁰¹—Y¹⁰²) is a carbene ligand. In some embodiments, Met is selected from Ir, Pt, Pd, Os, Cu, and Zn. In some embodiments, the metal complex has a smallest oxidation potential in solution vs. Fc+/Fc couple less than about 0.6 V.

In some embodiments, the HIL/HTL material is selected from the group consisting of phthalocyanine and porphryin compounds, starburst triarylamines, CFₓ fluorohydrocarbon polymer, conducting polymers (e.g., PEDOT:PSS, polyaniline, polypthiophene), phosphonic acid and silane SAMs, triarylamine or polythiophene polymers with conductivity dopants, Organic compounds with conductive inorganic compounds (such as molybdenum and tungsten oxides), n-type semiconducting organic complexes, metal organometallic complexes, cross-linkable compounds, polythiophene based polymers and copolymers, triarylamines, triaylamine with spirofluorene core, arylamine carbazole compounds, triarylamine with (di)benzothiophene/(di)benzofuran, indolocarbazoles, isoindole compounds, and metal carbene complexes.

### c) EBL:

An electron blocking layer (EBL) may be used to reduce the number of electrons and/or excitons that leave the emissive layer. The presence of such a blocking layer in a device may result in substantially higher efficiencies, and/or longer lifetime, as compared to a similar device lacking a blocking layer. Also, a blocking layer may be used to confine emission to a desired region of an OLED. In some embodiments, the EBL material has a higher LUMO (closer to the vacuum level) and/or higher triplet energy than one or more emitters closest to the EBL interface. In some embodiments, the compound used in EBL contains at least one carbazole group and/or at least one arylamine group. In some embodiments the HOMO level of the compound used in the EBL is shallower than the HOMO level of one or more of the hosts in the EML. In some embodiments, the compound used in EBL contains the same molecule or the same functional groups used as one of the hosts described herein.

### d) Hosts:

The light emitting layer of the organic EL device of the present disclosure preferably contains at least a light emitting material as the dopant, and a host material. Examples of the host material are not particularly limited, and any metal complexes or organic compounds may be used as long as the host won't fully quench the emission of the dopant.

Examples of metal complexes used as host are preferred to have the following general formula:

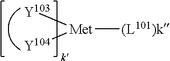

wherein Met is a metal; (Y¹⁰³_Y¹⁰⁴) is a bidentate ligand, the coordinating atoms of Y¹⁰³ and Y¹⁰⁴ are independently selected from C, N, O, P, and S; L¹⁰¹ is an another ligand; k′ is an integer value from 1 to the maximum number of ligands that may be attached to the metal; and k′+k″ is the maximum number of ligands that may be attached to the metal.

In some embodiments, the metal complexes are:

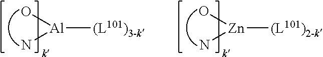

wherein (O—N) is a bidentate ligand, having metal coordinated to atoms O and N.

In some embodiments, Met is selected from Ir and Pt. In a further embodiment, (Y¹⁰³—Y¹⁰⁴) is a carbene ligand.

In some embodiments, the host compound contains at least one of the following groups selected from the group consisting of aromatic hydrocarbon cyclic compounds such as benzene, biphenyl, triphenyl, triphenylene, tetraphenylene, naphthalene, anthracene, phenalene, phenanthrene, fluorene, pyrene, chrysene, perylene, and azulene; the group consisting of aromatic heterocyclic compounds such as dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, aza-dibenzothiophene, aza-dibenzofuran, aza-dibenzoselenophene, aza-carbazole, aza-indolocarbazole, aza-triphenylene, aza-tetraphenylene, 5λ²-benzo[d]benzo[4,5]imidazo[3,2-a]imidazole, 5,9-dioxa-13b-boranaphtho[3,2,1-de]anthracene; and the group consisting of 2 to 10 cyclic structural units which are groups of the same type or different types selected from the aromatic hydrocarbon cyclic group and the aromatic heterocyclic group and are bonded to each other directly or via at least one of oxygen atom, nitrogen atom, sulfur atom, silicon atom, phosphorus atom, boron atom, chain structural unit and the aliphatic cyclic group. Each option within each group may be unsubstituted or may be substituted by the general substituents as described herein or may be further fused.

In some embodiments, the host compound comprises at least one of the moieties selected from the group consisting of:

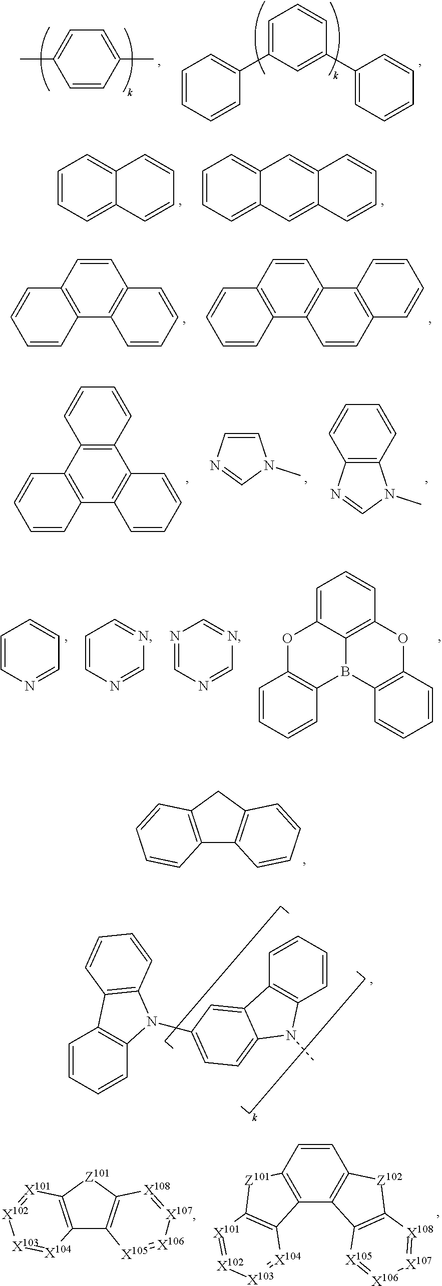

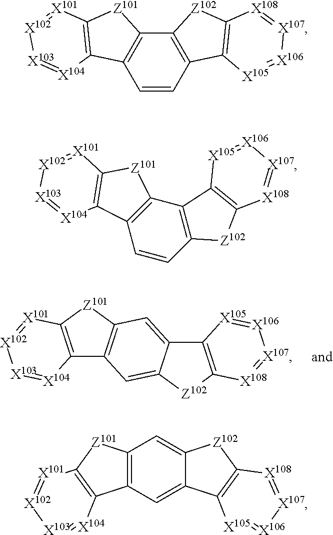

wherein k is an integer from 0 to 20 or 1 to 20. X¹⁰¹ to X¹⁰⁸ are independently selected from C or N. Z¹⁰¹ and Z¹⁰² are independently selected from C, N, O, or S.

In some embodiments, the host material is selected from the group consisting of arylcarbazoles, metal 8-hydroxyquinolates, (e.g., alq3, balq), metal phenoxybenzothiazole compounds, conjugated oligomers and polymers (e.g., polyfluorene), aromatic fused rings, zinc complexes, chrysene based compounds, aryltriphenylene compounds, poly-fused heteroaryl compounds, donor acceptor type molecules, dibenzofuran/dibenzothiophene compounds, polymers (e.g., pvk), spirofluorene compounds, spirofluorene-carbazole compounds, indolocabazoles, 5-member ring electron deficient heterocycles (e.g., triazole, oxadiazole), tetraphenylene complexes, metal phenoxypyridine compounds, metal coordination complexes (e.g., Zn, Al with N\hat{ }N ligands), dibenzothiophene/dibenzofuran-carbazole compounds, silicon/germanium aryl compounds, aryl benzoyl esters, carbazole linked by non-conjugated groups, aza-carbazole/dibenzofuran/dibenzothiophene compounds, and high triplet metal organometallic complexes (e.g., metal-carbene complexes).

### e) Emitter Materials in EML:

One or more additional emitter materials may be used in conjunction with the compound or device of the present disclosure. The one or more additional emitter materials can be emissive or non-emissive in the current device as described herein. For example, an additional emitter material that is a phosphorescent material can be used in an OLED device to provide a sensitizer function, and that same phosphorescent material can also be used in that OLED device as a light emitting phosphorescent emitter in another layer. Examples of the emitter materials are not particularly limited, and any compounds may be used as long as the compounds are capable of producing emissions in a regular OLED device. Examples of suitable additional emitter materials include, but are not limited to, compounds which are capable of producing emissions via phosphorescence, non-delayed fluorescence, delayed fluorescence, especially the thermally activated delayed fluorescence, i.e., TADF (also referred to as E-type delayed fluorescence), triplet-triplet annihilation, or combinations of these processes. Each of these materials suitable for the additional emitter materials has been described in the previous sections.

### f) HBL:

A hole blocking layer (HBL) may be used to reduce the number of holes and/or excitons that leave the emissive layer. The presence of such a blocking layer in a device may result in substantially higher efficiencies and/or longer lifetime as compared to a similar device lacking a blocking layer. Also, a blocking layer may be used to confine emission to a desired region of an OLED. In some embodiments, the HBL material has a lower HOMO (further away from the vacuum level) and/or higher triplet energy than one or more of the emitters closest to the HBL interface.

In some embodiments, a compound used in the HBL contains the same molecule or the same functional groups used as host described above.

In some embodiments, a compound used in the HBL comprises at least one of the following moieties selected from the group consisting of:

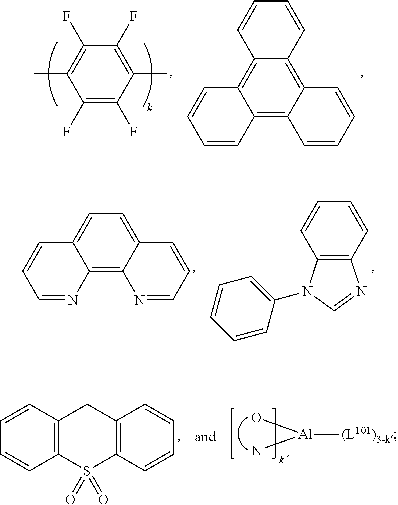

wherein k is an integer from 1 to 20; L¹⁰¹ is another ligand, k′ is an integer from 1 to 3.

### g) ETL:

Electron transport layer (ETL) may include a material capable of transporting electrons. Electron transport layer may be intrinsic (undoped), or doped. Doping may be used to enhance conductivity. Examples of the ETL material are not particularly limited, and any metal complexes or organic compounds may be used as long as they are typically used to transport electrons.

In some embodiments, compound used in ETL comprises at least one of the following moieties in the molecule:

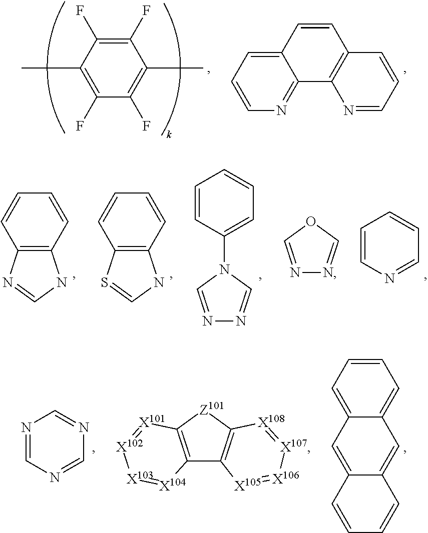

and fullerenes; wherein k is an integer from 1 to 20, X¹⁰¹ to X¹⁰⁸ is selected from C or N; Z¹⁰¹ is selected from the group consisting of C, N, O, and S.

In some embodiments, the metal complexes used in ETL contains, but not limit to the following general formula:

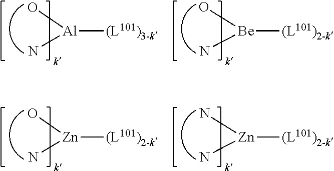

wherein (O—N) or (N—N) is a bidentate ligand, having metal coordinated to atoms O, N or N, N; L¹⁰¹ is another ligand; k′ is an integer value from 1 to the maximum number of ligands that may be attached to the metal.

In some embodiments, the ETL material is selected from the group consisting of anthracene-benzoimidazole compounds, aza triphenylene derivatives, anthracene-benzothiazole compounds, metal 8-hydroxyquinolates, metal hydroxybenoquinolates, bathocuprine compounds, 5-member ring electron deficient heterocycles (e.g., triazole, oxadiazole, imidazole, benzoimidazole), silole compounds, arylborane compounds, fluorinated aromatic compounds, fullerene (e.g., C60), triazine complexes, and Zn (N\hat{ }N) complexes.

### h) Charge Generation Layer (CGL)

In tandem or stacked OLEDs, the CGL plays an essential role in the performance, which is composed of an n-doped layer and a p-doped layer for injection of electrons and holes, respectively. Electrons and holes are supplied from the CGL and electrodes. The consumed electrons and holes in the CGL are refilled by the electrons and holes injected from the cathode and anode, respectively; then, the bipolar currents reach a steady state gradually. Typical CGL materials include n and p conductivity dopants used in the transport layers.

In any compounds disclosed herein, the hydrogen atoms can be partially or fully deuterated. The minimum amount of hydrogen of the compound being deuterated is selected from the group consisting of 30%, 40%, 50%, 60%, 70%, 80%, 90%, 95%, 99%, and 100%. As used herein, percent deuteration has its ordinary meaning and includes the percent of all possible hydrogen and deuterium atoms that are replaced by deuterium atoms. In some embodiments, the deuterium atoms are attached to an aromatic ring. In some embodiments, the deuterium atoms are attached to a saturated carbon atom, such as an alkyl or cycloalkyl carbon atom. In some other embodiments, the deuterium atoms are attached to a heteroatom, such as Si, or Ge atom.

It is understood that the various embodiments described herein are by way of example only and are not intended to limit the scope of the invention. For example, many of the materials and structures described herein may be substituted with other materials and structures without deviating from the spirit of the invention. The present invention as claimed may therefore include variations from the particular examples and preferred embodiments described herein, as will be apparent to one of skill in the art. It is understood that various theories as to why the invention works are not intended to be limiting.

It should also be understood that embodiments of all the compounds and devices described herein may be interchangeable if those embodiments are also applicable under different aspects of the entire disclosure.

### E. Experimental Section

### Synthesis of Inventive Compound-1

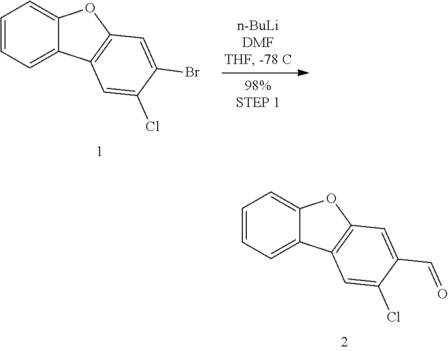 [SMILES: Clc1cc2c(cc1Br)oc1ccccc12 ; O=Cc1cc2oc3ccccc3c2cc1Cl]

Synthesis of 2-chlorodibenzo[b,d]furan-3-carbaldehyde (2): A solution of 3-bromo-2-chlorodibenzo[b,d]furan (1) (0.87 g, 1 Eq, 3.1 mmol) in tetrahydrofuran (15 mL) was cooled to −78 C in dry ice acetone bath and n-butyllithium (0.22 g, 1.1 Eq, 3.4 mmol) was added and stirred for 15 minutes. N,N-dimethylformamide (0.45 g, 2 Eq., 6.2 mmol) was added slowly and stirred for 15 minutes. The reaction mixture was warmed to room temperature and quenched with 1 M hydrochloric acid (5 mL) and stirred for 15 minutes. The reaction mixture was diluted with DCM and washed with water (100 mL), the organic fraction was dried over sodium sulfate and evaporated to dryness to give compound 3 (0.7 g, 98% yield) as a white solid.

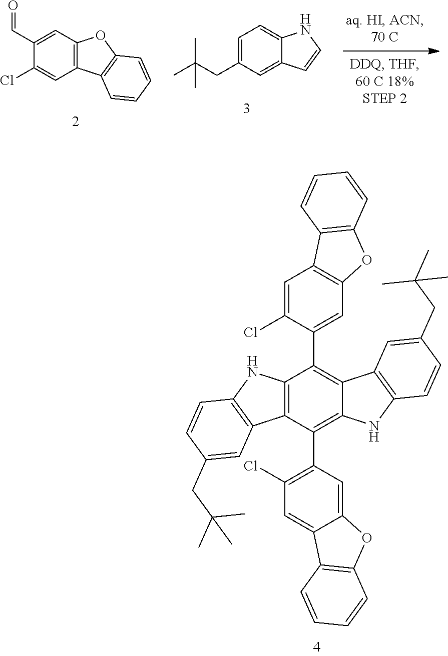

Synthesis of 6,12-bis(2-chlorodibenzo[b,d]furan-3-yl)-2,8-dineopentyl-5,11-dihydroindolo[3,2-b]carbazole (4): 2-chlorodibenzo[b,d]furan-3-carbaldehyde (2) (0.67 g, 1 Eq, 2.9 mmol) and 5-neopentyl-1H-indole (3) (0.544 g, 1 Eq., 2.9 mmol) were suspended in acetonitrile (30 mL) and hydroiodic acid (0.65 g, 1 Eq., 2.9 mmol, 57% aq. solution) was added in one portion. The reaction was stirred for 2 hours at 70° C. at which point the reaction was cooled to room temperature, diluted with methanol (30 mL) and the precipitate was filtered to give fine beige solid (0.38 g). The solid was dissolved in THF (15 mL) and 4,5-dichloro-3,6-dioxocyclohexa-1,4-diene-1,2-dicarbonitrile (216 mg, 2 Eq., 0.9 mmol) was added in one portion and the reaction mixture was heated to 60° C. for 2 hours. The reaction mixture was cooled to room temperature and diluted with methanol (30 mL) and stirred for 15 minutes. The solids were filtered and air dried to give 6,12-bis(2-chlorodibenzo[b,d]furan-3-yl)-2,8-dineopentyl-5,11-dihydroindolo[3,2-b]carbazole 4 (70 mg, 18% yield) as a pale yellow solid.

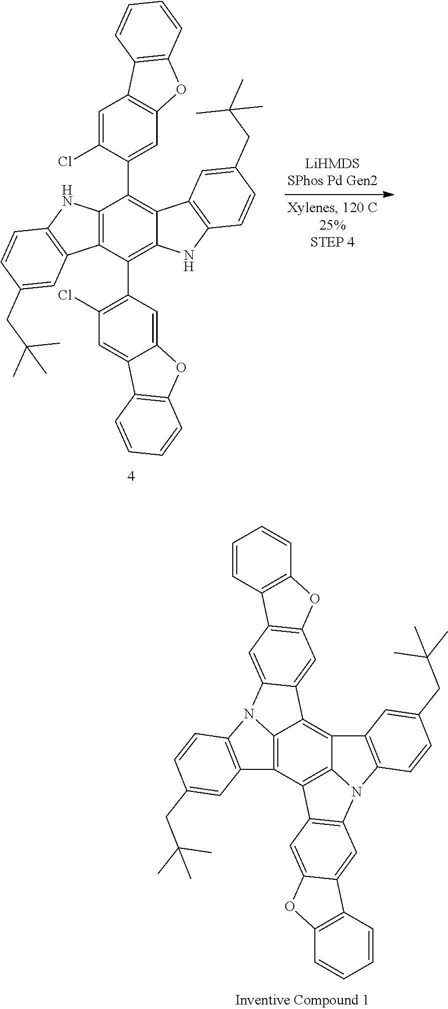 [SMILES: CC(C)(C)Cc1ccc2[nH]c3c(-c4cc5oc6ccccc6c5cc4Cl)c4c([nH]c5ccc(CC(C)(C)C)cc54)c(-c4cc5oc6ccccc6c5cc4Cl)c3c2c1 ; CC(C)(C)Cc1ccc2c(c1)c1c3c4cc5oc6ccccc6c5cc4n4c5ccc(CC(C)(C)C)cc5c(c5c6cc7oc8ccccc8c7cc6n2c15)c34]

Synthesis of 2,14-dineopentylbenzo[5,6]benzofuro[3″,2″:4′,5′]benzo[1′,2′:2,3]pyrrolizino[1,7-bc]benzofuro[2,3-h]indolo[1,2,3-lm]carbazole (Inventive Compound-1): 6,12-bis(2-chlorodibenzo[b,d]furan-3-yl)-2,8-dineopentyl-5,11-dihydroindolo[3,2-b]carbazole (30 mg, 1 Eq., 0.04 mmol) was suspended in anhydrous xylene (2 mL) and LHMDS (13 mg, 2.1 Eq., 0.08 mmol) was added slowly and stirred for 5 minutes at room temperature. SPhos Pd Gen2 (5 mg, 0.2 Eq., 0.008 mmol) was added in one portion, the reaction mixture was sparged with nitrogen for 1 minute and heated to 120° C. for 16 hours at which point the reaction mixture was cooled to room temperature, quenched with methanol (1 mL) and evaporated to dryness. The solid residue was suspended in o-dichlorobenzene (10 mL), refluxed for 5 minutes and cooled to room temperature. The solution was wet loaded on a silica gel column (Sorbtech, 120 g), equipped with a celite plug and eluted with dichloromethane to collect the yellow fractions. The fractions were evaporated to dryness and the solid material was suspended in o-dichlorobenzene (4 mL) and refluxed for 5 minutes to form a clear solution which was allowed cool to room temperature and to from a precipitate. The bright yellow precipitate was filtered off and washed with DCM and dried to give 2,14-dineopentylbenzo[5,6]benzofuro[3″,2″:4′,5′]benzo[1′,2′: 2,3]pyrrolizino[1,7-bc]benzofuro[2,3-h]indolo[1,2,3-lm]carbazole (Inventive Compound-1) as a fine yellow crystalline powder (7 mg, 25% yield).

Inventive compound 1 emits at room temperature in fluid 2-MeTHF solution with an emission λₘₐₓ=479 nm. The emission profile is narrow and structured with a fwhm=14 nm (42 meV), which is well-suited for display applications with high color purity.

The emission spectrum was collected on a Horiba Fluorolog-3 spectrofluorometer equipped with a Synapse Plus CCD detector. The sample was excited at 340 nm.

What is claimed is:

1. A compound of Formula I: 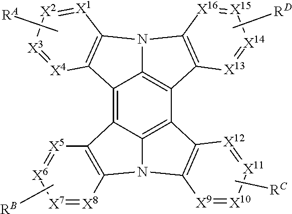
wherein each of X¹ to X¹⁶ is independently C or N;
each of R^{A}, R^{B}, R^{C}, and R^{D} independently represents mono to the maximum allowable substitutions, or no substitutions;
wherein any two of R^{A}, R^{B}, R^{C}, and R^{D} may be joined or fused to form a ring;
with the proviso that, if the compound comprises a structure of Formula II: 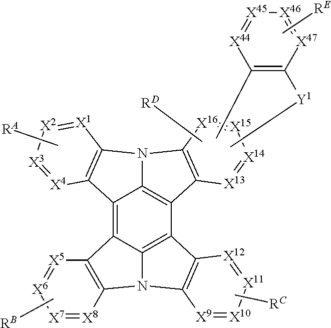
wherein each of X⁴⁴ to X⁴ is independently C or N;
wherein Y¹ is selected from the group consisting of O, NR^{Y1}, and CMe₂;
wherein R^{E} represents mono to the maximum allowable substitutions, or no substitutions;
then
(i) when Y¹ is O or CMe₂, then X¹⁵ is C, and
(ii) when Y¹ is NR^{Y1}, then R^{Y1} and R^{E} do not join to form a 5-membered ring;
with the proviso that, if no two of R^{A}, two of R^{B}, two of R^{C}, or two of R^{D} are joined to form a ring, then at least one of R^{A}, R^{B}, R^{C}, or R^{D} comprises a structure selected from the group consisting of deuterium, alkyldiarylsilyl, dialkylarylsilyl, germyl, tetraphenylene, a bridged polycyclic ring, an alkyl or cycloalkyl comprising five or more carbons, and an aromatic ring fused by a non-aromatic ring;
with the proviso that, if two of R^{A}, two of R^{B}, two of R^{C}, or two of R^{D} are joined to form a benzene ring, then at least one of R^{A}, R^{B}, R^{C}, or R^{D} comprises a structure selected from the group consisting of deuterium, silyl, germyl, tetraphenylene, benzo[d]benzo[4,5]imidazo[1,2-a]imidazole (bimbim), a bridged polycyclic ring, an alkyl or cycloalkyl comprising five or more carbons, an aromatic ring fused by a non-aromatic ring, Formula III, and Formula IV, 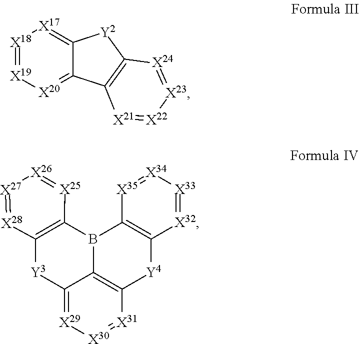
wherein each of X¹⁷ to X³⁵ is independently C or N, and
each Y², Y³, and Y⁴ is independently selected from the group consisting of O, S, Se, NR^{Y2}, BR^{Y2}, BR^{Y2}R^{Y3}, CR^{Y2}R^{Y3}, SiR^{Y2}R^{Y3}, GeR^{Y2}R^{Y3}, C═O, C═S, C═NR^{Y2}, C═CR^{Y2}R^{Y3}, SO, SO₂, or P(O)R^{Y2} with the proviso that, if two of R^{A}, two of R^{B}, two of R^{C}, or two of R^{D} are joined to form an 5 or 6-membered cycloalkyl ring, then at least one of R^{A}, R^{B}, R^{C}, or R^{D} comprises a structure selected from the group consisting of deuterium, alkyldiarylsilyl, dialkylarylsilyl, trirylsilane, substituted triphenylsilyl, germyl, tetraphenylene, benzo[d]benzo[4,5]imidazo[1,2-a]imidazole, a bridged polycyclic ring, an alkyl or cycloalkyl comprising five or more carbons, an aromatic ring fused by a non-aromatic ring, and Formula V: 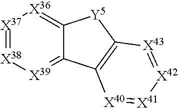
wherein each of X³⁶ to X⁴³ is independently C or N, and
wherein Y⁵ is selected from the group consisting of O, S, Se, NR^{Y4}, BR^{Y4}, BR^{Y4}R^{Y5}, CR^{Y4}R^{Y5}, SiR⁴R^{Y5}, GeR^{Y4}R^{Y5}, C═O, C═S, C═NR^{Y4}, C═CR^{Y4}R^{Y5}, SO, SO₂, or P(O)R^{Y4};
wherein each of R^{Y1}, R^{Y2}, R^{Y3}, R^{Y4}, R^{Y5}, R^{A}, R^{B}, R^{C}, R^{D}, and R^{E} is independently hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof;
wherein the compound is not:

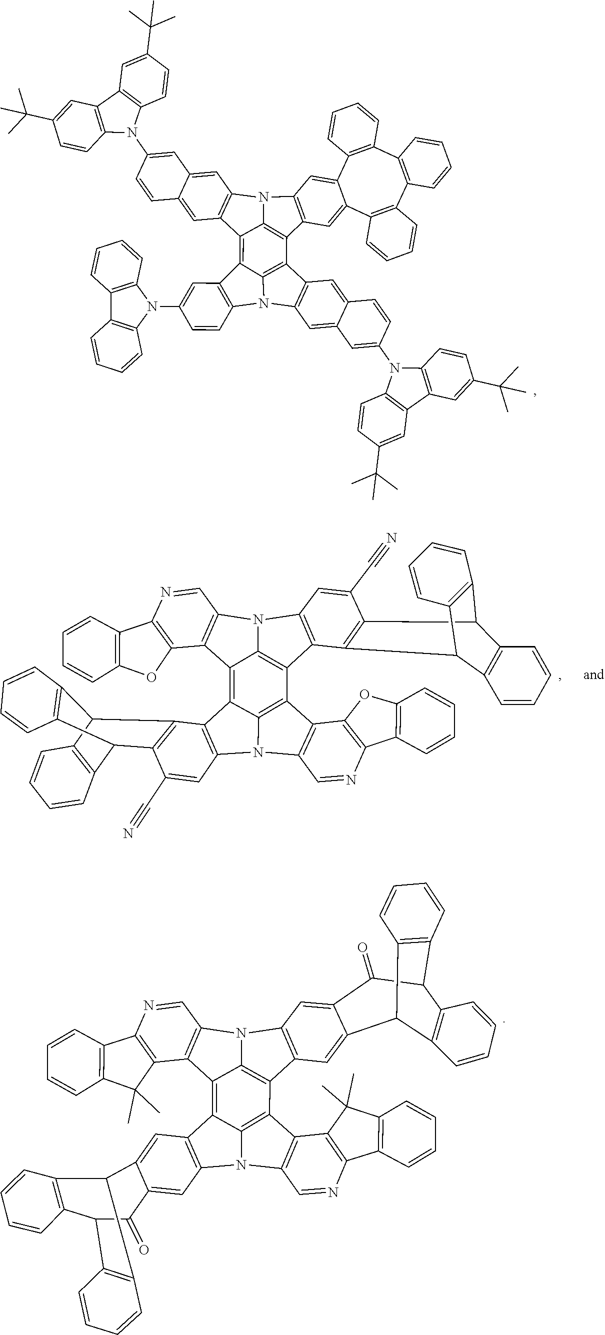

2. The compound of claim 1, wherein the compound is fully or partially deuterated.

3. The compound of claim 1, wherein each of R^{Y1}, R^{Y2}, R^{Y3}, R^{Y4}, R^{Y5}, R^{A}, R^{B}, R^{C}, R^{D}, and R^{E} is independently a hydrogen or a substituent selected from the group consisting of deuterium, fluorine, alkyl, cycloalkyl, heteroalkyl, alkoxy, aryloxy, amino, silyl, germyl, boryl, alkenyl, cycloalkenyl, heteroalkenyl, aryl, heteroaryl, nitrile, isonitrile, sulfanyl, and combinations thereof.

4. The compound of claim 1, wherein at least one of X¹ to X¹⁶ is N.

5. The compound of claim 1, wherein all of X¹ to X¹⁶ are C.

6. The compound of claim 1, wherein the compound has a structure of Formula II and is selected from the group consisting of the structures from LIST 1a as defined herein;
wherein Y¹ to Y¹⁸ is independently selected from the group consisting of O, S, Se, NR^{Y*}, BR^{Y*}, BR^{Y*}R^{Y*}, CR^{Y*}R^{Y*′}, SiR^{Y*}R^{Y*′}, GeR^{Y*}R^{Y*′}, C═O, C═S, C═NR^{Y*}, C═CR^{Y*}R^{Y*′}, SO, SO₂, or P(O)R^{Y*};
wherein R^{Y*} and R^{Y*′} is independently hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof; and
wherein X⁴⁸ to X⁵⁰ is independently C or N.

7. The compound of claim 1, wherein no two of R^{A}, two of R^{B}, two of R^{C}, or two of R^{D} are joined to form a ring, and at least one of R^{A}, R^{B}, R^{C}, or R^{D} comprises a structure selected from the group consisting of deuterium, alkyldiarylsilyl, dialkylarylsilyl, germyl, tetraphenylene, an alkyl or cycloalkyl comprising five or more carbons, and an aromatic ring fused by a non-aromatic ring.

8. The compound of claim 1, wherein two of R^{A}, two of R^{B}, two of R^{C}, or two of R^{D} are joined to form a benzene ring, and at least one of R^{A}, R^{B}, R^{C}, or R^{D} comprises a structure selected from the group consisting of deuterium, silyl, germyl, tetraphenylene, benzo[d]benzo[4,5]imidazo[1,2-a]imidazole (bimbim), an alkyl or cycloalkyl comprising five or more carbons, an aromatic ring fused by a non-aromatic ring, Formula III, and Formula IV.

9. The compound of claim 8, wherein the compound is selected from the group consisting of the following structures (LIST 1c) and at least one of R^{A}, R^{B}, R^{C}, or R^{D} comprises a structure selected from the group consisting of deuterium, silyl, germyl, tetraphenylene, benzo[d]benzo[4,5]imidazo[1,2-a]imidazole (bimbim), an alkyl or cycloalkyl comprising five or more carbons, an aromatic ring fused by a non-aromatic ring, Formula III, and Formula IV:

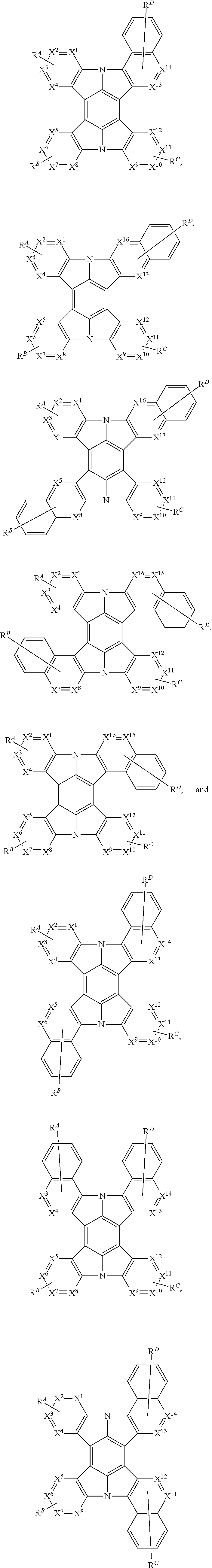

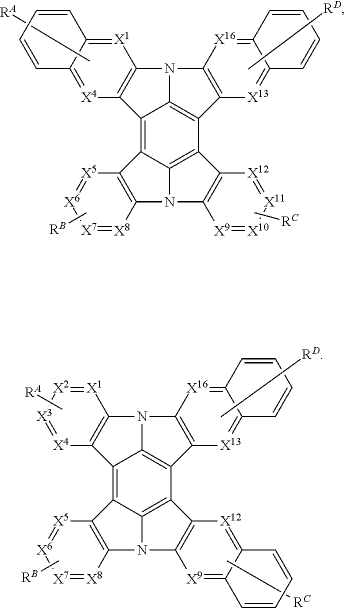

10. The compound of claim 1, wherein two of R^{A}, two of R^{B}, two of R^{C}, or two of R^{D} are joined to form an 5 or 6-membered cycloalkyl ring, and at least one of R^{A}, R^{B}, R^{C}, or R^{D} comprises a structure selected from the group consisting of deuterium, alkyldiarylsilyl, dialkylarylsilyl, triarylsilane, substituted triphenylsilyl, germyl, tetraphenylene, benzo[d]benzo[4,5]imidazo[1,2-a]imidazole (bimbim), an alkyl or cycloalkyl comprising five or more carbons, an aromatic ring fused by a non-aromatic ring, and Formula V.

11. The compound of claim 10, wherein the compound is selected from the group consisting of the following structures (LIST 1d), and at least one of R^{A}, R^{B}, R^{C}, or R^{D} comprises a structure selected from the group consisting of deuterium, alkyldiarylsilyl, dialkylarylsilyl, triarylsilane, substituted triphenylsilyl, germyl, tetraphenylene, benzo[d]benzo[4,5]imidazo[1,2-a]imidazole (bimbim), an alkyl or cycloalkyl comprising five or more carbons, an aromatic ring fused by a non-aromatic ring, and Formula V:

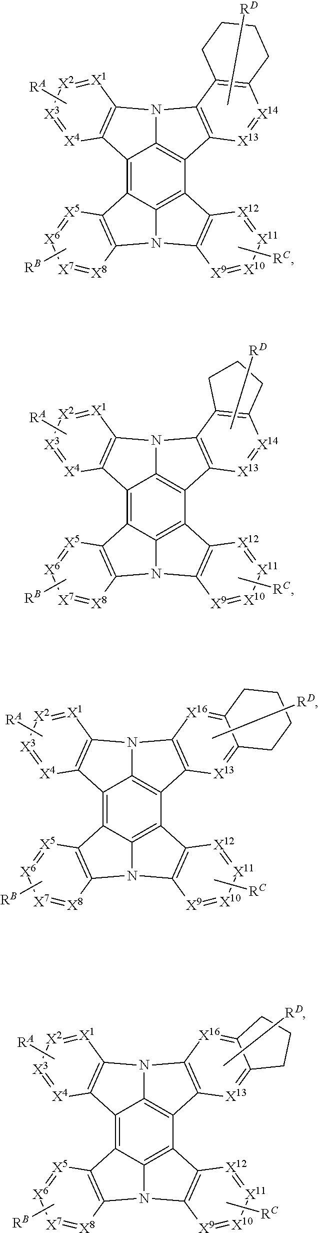

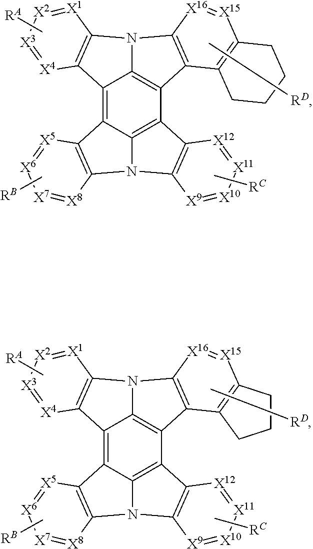

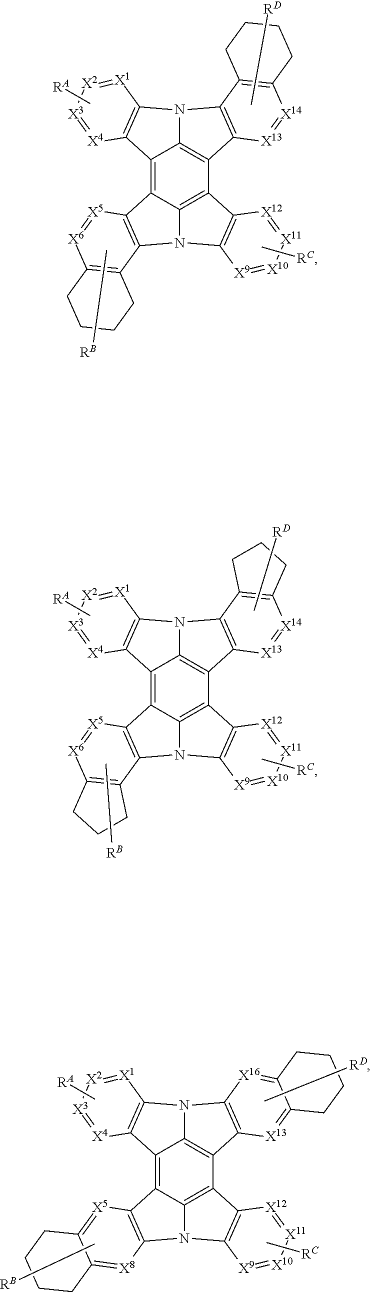

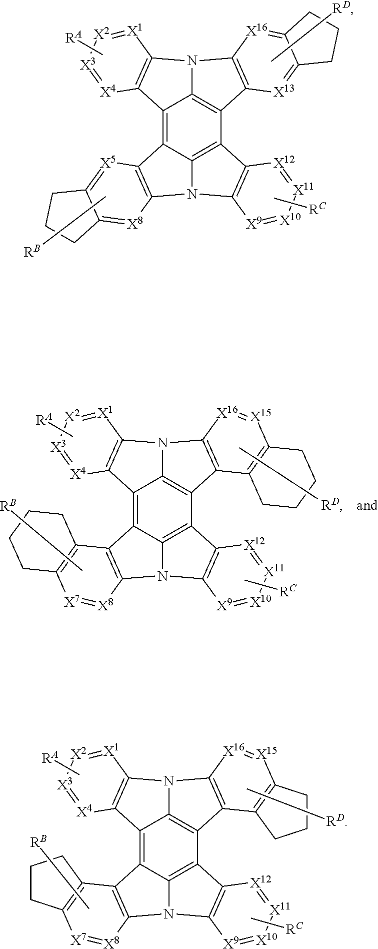

12. The compound of claim 1, wherein the compound has an E(S₁)-E(T₁) equal to or higher than 0.3 ev.

13. The compound of claim 1, wherein the compound is selected from the group consisting of Compound W1-(Ri)(Rj)(Rk)(Rl)(Ym), Compound W2-(Ri)(Rj)(Rk)(Rl)(Ym)(Yn), Compound W3-(Ri)(Rj)(Rl)(Ym)(Yn), Compound W4-(Ri)(Rj)(Rk)(Rl)(Ym), Compound W5-(Rj)(Rk)(Rl)(Rq), Compound W6-(Rj)(Rk)(Rl)(Rr), Compound W7-(Rj)(Rk)(Rl)(Rs), Compound W8-(Ri)(Rj)(Rk)(Rl), and Compound W9-(Ri)(Rj)(Rn′), wherein W1 is an integer from 1 to 14, W2 is an integer from 15 to 46, 49, 50, 53-60, and 93-144, W3 is 47, 48, 51, and 52, W4 is an integer from 61-92, W5 is an integer from 145-154, W6 is an integer from 155-222, W7 is an integer from 223-234, W8 is an integer from 235-341, and W9 is 342, i, j, k, n′, and l are an integer from 1 to 468; m and n are an integer from 1 to 51, q is an integer from 1 to 177, r is an integer from 1 to 232, s is an integer from 1 to 209, each of Ri, Rj, Rk, Rn′, and Rl is independently selected from the group consisting of R1 to R⁴⁶⁸; each of Rq is selected from the group consisting of R1 to R177; each of Rr is selected from the group consisting of R1 to R²³²; each of Rs is selected from the group consisting of R1 to R²⁰⁹; each of Ym and Yn is independently selected from the group consisting of Y1 to Y51;
wherein each of the compounds are defined in LIST 2 as defined herein;
wherein Y1 to Y51 have the structures as defined in LIST 15 as defined herein;
wherein R1 to R⁴⁶⁸ have the structures as defined in LIST 3 as defined herein.

14. The compound of claim 1, wherein the compound is selected from the group consisting of LIST 4 as defined herein.

15. An organic light emitting device (OLED) comprising:
an anode;
a cathode; and
an emissive region disposed between the anode and the cathode,
wherein the emissive region comprises a compound comprising a structure of Formula I: 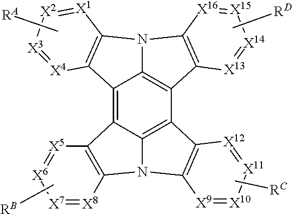
wherein each of X¹ to X¹⁶ is independently C or N;
each of R^{A}, R^{B}, R^{C}, and R^{D} independently represents mono to the maximum allowable substitutions, or no substitutions;
wherein any two of R^{A}, R^{B}, R^{C}, and R^{D} may be joined or fused to form a ring;
with the proviso that, if the compound comprises a structure of Formula II: 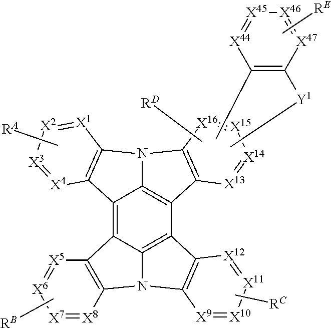
wherein each of X⁴⁴ to X⁴ is independently C or N;
wherein Y¹ is selected from the group consisting of O, NR^{Y1}, and CMe₂;
wherein R^{E} represents mono to the maximum allowable substitutions, or no substitutions;
then
(i) when Y¹ is O or CMe₂, then X⁵ is C, and
(ii) when Y¹ is NR^{Y1}, then R^{Y1} and R^{E} do not join to form a 5-membered ring;
with the proviso that, if no two of R^{A}, two of R^{B}, two of R^{C}, or two of R^{D} are joined to form a ring, then at least one of R^{A}, R^{B}, R^{C}, or R^{D} comprises a structure selected from the group consisting of deuterium, alkyldiarylsilyl, dialkylarylsilyl, germyl, tetraphenylene, an alkyl or cycloalkyl comprising five or more carbons, and an aromatic ring fused by a non-aromatic ring;
with the proviso that, if two of R^{A}, two of R^{B}, two of R^{C}, or two of R^{D} are joined to form a benzene ring, then at least one of R^{A}, R^{B}, R^{C}, or R^{D} comprises a structure selected from the group consisting of deuterium, silyl, germyl, tetraphenylene, benzo[d]benzo[4,5]imidazo[1,2-a]imidazole (bimbim), an alkyl or cycloalkyl comprising five or more carbons, an aromatic ring fused by a non-aromatic ring, Formula III, and Formula IV, 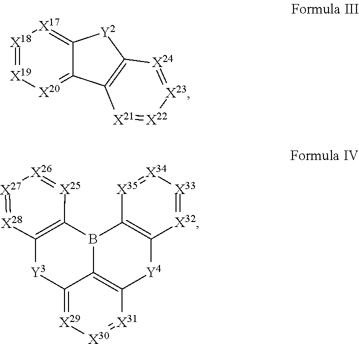
wherein each of X¹⁷ to X³⁵ is independently C or N, and
each Y², Y³, and Y⁴ is independently selected from the group consisting of O, S, Se, NR^{Y2}, BR^{Y2}, BR^{Y2}R^{Y3}, CR^{Y2}R^{Y3}, SiR^{Y2}R^{Y3}, GeR^{Y2}R^{Y3}, C═O, C═S, C═NR^{Y2}, C═CR^{Y2}R^{Y3}, SO, SO₂, or P(O)R^{Y2} with the proviso that, if two of R^{A}, two of R^{B}, two of R^{C}, or two of R^{D} are joined to form an 5 or 6-membered cycloalkyl ring, then at least one of R^{A}, R^{B}, R^{C}, or R^{D} comprises a structure selected from the group consisting of deuterium, alkyldiarylsilyl, dialkylarylsilyl, triarylsilane, substituted triphenylsilyl, germyl, tetraphenylene, benzo[d]benzo[4,5]imidazo[1,2-a]imidazole, an alkyl or cycloalkyl comprising five or more carbons, an aromatic ring fused by a non-aromatic ring, and Formula V: 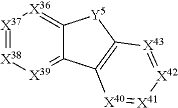
wherein each of X³⁶ to X⁴³ is independently C or N, and
wherein Y⁵ is selected from the group consisting of O, S, Se, NR^{Y4}, BR^{Y4}, BR^{Y4}R^{Y5}, CR^{Y4}R^{Y5}, SiR^{Y4}R^{Y5}, GeR^{Y4}R^{Y5}, C═O, C═S, C═NR^{Y4}, C═CR^{Y4}R^{Y5}, SO, SO₂, or P(O)R^{Y4};
wherein each of R^{Y1}, R^{Y2}, R^{Y3}, R^{Y4}, R^{Y5}, R^{A}, R^{B}, R^{C}, R^{D}, and R^{E} is independently hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof;
wherein the compound is not:

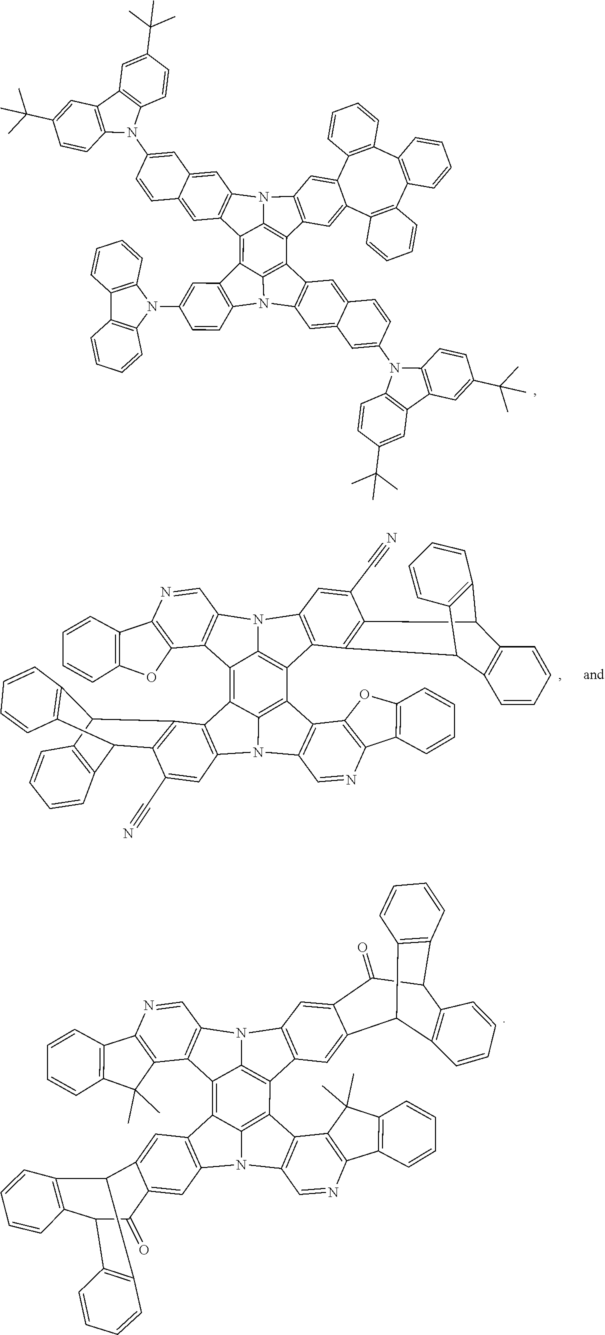

16. The OLED of claim 15, wherein the compound is a host or wherein the compound is a fluorescent emitter.

17. The OLED of claim 15, wherein the emissive region further comprises a compound S1;
wherein the compound comprises the structure of Formula I is an acceptor (A1);
wherein the compound S1 is a sensitizer capable of energy transfer from at least one of its excited states to an excited state of the acceptor A1 at room temperature.

18. The OLED of claim 17, wherein the compound A1 is selected from the group consisting of the structures in LIST 1a; or wherein the compound A1 is selected from the group consisting of the structures in LIST 1c; or wherein the compound A1 is selected from the group consisting of the structures in LIST 1d; or wherein the compound A1 is selected from the group consisting of the structures in LIST 2.

19. A consumer product comprising the OLED according to claim 15.

20. A compound, or a neutral molecular form thereof, or a monovalent or polyvalent form thereof, or a monomeric or polymeric form thereof, or a macromolecular or supramolecular form thereof; wherein the compound has a structure of Formula I 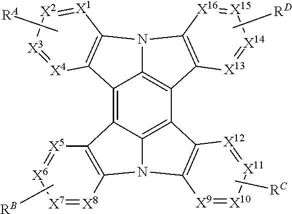
wherein each of X¹ to X¹⁶ is independently C or N;
each of R^{A}, R^{B}, R^{C}, and R^{D} independently represents mono to the maximum allowable substitutions, or no substitutions;
wherein any two of R^{A}, R^{B}, R^{C}, and R^{D} may be joined or fused to form a ring;
with the proviso that, if the compound comprises a structure of Formula II: 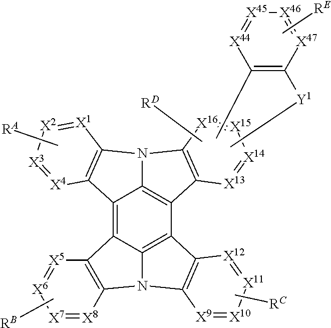
wherein each of X⁴⁴ to X⁴ is independently C or N;
wherein Y¹ is selected from the group consisting of O, NR^{Y1}, and CMe₂;
wherein R^{E} represents mono to the maximum allowable substitutions, or no substitutions;
then
(i) when Y¹ is O or CMe₂, then X¹⁵ is C, and
(ii) when Y¹ is NR^{Y1}, then R^{Y1} and R^{E} do not join to form a 5-membered ring;
with the proviso that, if no two of R^{A}, two of R^{B}, two of R^{C}, or two of R^{D} are joined to form a ring, then at least one of R^{A}, R^{B}, R^{C}, or R^{D} comprises a structure selected from the group consisting of deuterium, alkyldiarylsilyl, dialkylarylsilyl, germyl, tetraphenylene, an alkyl or cycloalkyl comprising five or more carbons, and an aromatic ring fused by a non-aromatic ring;
with the proviso that, if two of R^{A}, two of R^{B}, two of R^{C}, or two of R^{D} are joined to form a benzene ring, then at least one of R^{A}, R^{B}, R^{C}, or R^{D} comprises a structure selected from the group consisting of deuterium, silyl, germyl, tetraphenylene, benzo[d]benzo[4,5]imidazo[1,2-a]imidazole (bimbim), an alkyl or cycloalkyl comprising five or more carbons, an aromatic ring fused by a non-aromatic ring, Formula III, and Formula IV, 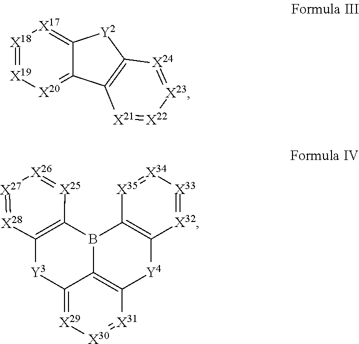
wherein each of X¹⁷ to X³⁵ is independently C or N, and
each Y², Y³, and Y⁴ is independently selected from the group consisting of O, S, Se, NR^{Y2}, BR^{Y2}, BR^{Y2}R^{Y3}, CR^{Y2}R^{Y3}, SiR^{Y2}R^{Y3}, GeR^{Y2}R^{Y3}, C═O, C═S, C═NR^{Y2}, C═CR^{Y2}R^{Y3}, SO, SO₂, or P(O)R^{Y2};
with the proviso that, if two of R^{A}, two of R^{B}, two of R^{C}, or two of R^{D} are joined to form an 5 or 6-membered cycloalkyl ring, then at least one of R^{A}, R^{B}, R^{C}, or R^{D} comprises a structure selected from the group consisting of deuterium, alkyldiarylsilyl, dialkylarylsilyl, triarylsilane, substituted triphenylsilyl, germyl, tetraphenylene, benzo[d]benzo[4,5]imidazo[1,2-a]imidazole, an alkyl or cycloalkyl comprising five or more carbons, an aromatic ring fused by a non-aromatic ring, and Formula V: 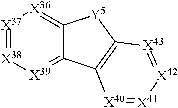
wherein each of X³⁶ to X⁴³ is independently C or N, and
wherein Y⁵ is selected from the group consisting of O, S, Se, NR^{Y4}, BR^{Y4}, BR^{Y4}R^{Y5}, CR^{Y4}R^{Y5}, SiR^{Y4}R^{Y5}, GeR^{Y4}R^{Y5}, C═O, C═S, C═NR^{Y4}, C═CR^{Y4}R^{Y5}, SO, SO₂, or P(O)R^{Y4};
wherein each of R^{Y1}, R^{Y2}, R^{Y3}, R^{Y4}, R^{Y5}, R^{A}, R^{B}, R^{C}, R^{D}, and R^{E} is independently hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof;
wherein the compound is not: [chemical structure]
